SEQUENCE LISTING (27 sequences)

SEQ ID NO: 1 - protein, 5 aa, Artificial Sequence
  Synthetic
YGGFM

SEQ ID NO: 2 - protein, 5 aa, Artificial Sequence
  Synthetic
YGGFL

SEQ ID NO: 3 - protein, 6 aa, Artificial Sequence
  Synthetic
  MISC_FEATURE (1)..(1): X is N, N-diallyl-Tyrosine
  MISC_FEATURE (3)..(4): X is Aib (aminoisobutytic acid)
  L (6)..(6): L is Leu-OH
XYXXFL

SEQ ID NO: 4 - protein, 8 aa, Artificial Sequence
  Synthetic
  F (1)..(1): F at position 1 is D-Phe
  MISC_FEATURE (7)..(7): X is Pen (Pencillamine)
  T (8)..(8): T is Thr-NH2
FCYWKTXT

SEQ ID NO: 5 - protein, 50 aa, Artificial Sequence
  Synthetic
QSALDYFMFAVRCRHQRRQLVHFAWEHFRPRCKFVWGPQDKLRRFKPSSL

SEQ ID NO: 6 - protein, 7 aa, Simian virus 40
PKKKRKV

SEQ ID NO: 7 - protein, 10 aa, Simian virus 40
PKKKRKVEAS

SEQ ID NO: 8 - protein, 16 aa, Artificial Sequence
  Synthetic
KRPAATKKAGQAKKKK

SEQ ID NO: 9 - protein, 9 aa, Artificial Sequence
  Synthetic
PAAKRVKLD

SEQ ID NO: 10 - protein, 11 aa, Artificial Sequence
  Synthetic
RQRRNELKRSP

SEQ ID NO: 11 - protein, 38 aa, Artificial Sequence
  Synthetic
NQSSNFGPMKGGNFGGRSSGPYGGGGQYFAKPRNQGGY

SEQ ID NO: 12 - protein, 42 aa, Artificial Sequence
  Synthetic
RMRIZFKNKGKDTAELRRRRVEVSVELRKAKKDEQILKRRNV

SEQ ID NO: 13 - protein, 8 aa, Artificial Sequence
  Synthetic
VSRKRPRP

SEQ ID NO: 14 - protein, 8 aa, Artificial Sequence
  Synthetic
PPKKARED

SEQ ID NO: 15 - protein, 8 aa, Homo sapiens
PQPKKKPL

SEQ ID NO: 16 - protein, 12 aa, Mus sp.
SALIKKKKKMAP

SEQ ID NO: 17 - protein, 5 aa, Influenza virus
DRLRR

SEQ ID NO: 18 - protein, 7 aa, Influenza virus
PKQKKRK

SEQ ID NO: 19 - protein, 10 aa, Hepatitis virus
RKLKKKIKKL

SEQ ID NO: 20 - protein, 10 aa, Mus sp.
REKKKFLKRR

SEQ ID NO: 21 - protein, 20 aa, Homo sapiens
KRKGDEVDGVDEVAKKKSKK

SEQ ID NO: 22 - protein, 17 aa, Homo sapiens
RKCLQAGMNLEARKTKK

SEQ ID NO: 23 - protein, 267 aa, Artificial Sequence
  Synthetic
MARFLTLCTWLLLLGPGLLATVRAECSQDCATCSYRLVRPADINFLACVMECEGKLPSLKIWETCKELLQLSKPELPQDGTSTLRENSKPEESHLLAKRYGGFMKRYGGFMKKMDELYPMEPEEEANGSEILAKRYGGFMKKDAEEDDSLANSSDLLKELLETGDNRERSHHQDGSDNEEEVSKRYGGFMRGLKRSPQLEDEAKELQKRYGGFMRRVGRPEWWMDYQKRYGGFLKRFAEALPSDEEGESYSKEVPEMEKRYGGFMRF

SEQ ID NO: 24 - protein, 677 aa, Artificial Sequence
  Synthetic
MDDPDCDSTWEEDEEDAEDAEDEDCEDGEAAGARDADAGDEDEESEEPRAARPSSFQSRMTGSRNWRATRDMCRYRHNYPDLVERDCNGDTPNLSFYRNEIRFLPNGCFIEDILQNWTDNYDLLEDNHSYIQWLFPLREPGVNWHAKPLTLREVEVFKSSQEIQERLVRAYELMLGFYGIRLEDRGTGTVGRAQNYQKRFQNLNWRSHNNLRITRILKSLGELGLEHFQAPLVRFFLEETLVRRELPGVRQSALDYFMFAVRCRHQRRQLVHFAWEHFRPRCKFVWGPQDKLRRFKPSSLPHPLEGSRKVEEEGSPGDPDHEASTQGRTCGPEHSKGGGRVDEGPQPRSVEPQDAGPLERSQGDEAGGHGEDRPEPLSPKESKKRKLELSRREQPPTEPGPQSASEVEKIALNLEGCALSQGSLRTGTQEVGGQDPGEAVQPCRQPLGARVADKVRKRRKVDEGAGDSAAVASGGAQTLALAGSPAPSGHPKAGHSENGVEEDTEGRTGPKEGTPGSPSETPGPSPAGPAGDEPAESPSETPGPRPAGPAGDEPAESPSETPGPRPAGPAGDEPAESPSETPGPSPAGPTRDEPAESPSETPGPRPAGPAGDEPAESPSETPGPRPAGPAGDEPAESPSETPGPSPAGPTRDEPAKAGEAAELQDAEVESSAKSGKP

SEQ ID NO: 25 - protein, 107 aa, Artificial Sequence
  Synthetic
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 26 - protein, 9 aa, Artificial Sequence
  Synthetic
IEVMYPPPY

SEQ ID NO: 27 - protein, 107 aa, Artificial Sequence; identical to SEQ ID NO: 25
  Synthetic

(19) United States
(12) Patent Application Publication
(10) Pub. No.: US 2022/0105135 A1
(43) Pub. Date: Apr. 7, 2022

(54) METHODS AND COMPOSITIONS FOR THE MODULATION OF OPIOID SIGNALING IN THE TUMOR MICROENVIRONMENT

(71) Applicants: The Broad Institute, Inc., Cambridge, MA (US); Dana-Farber Cancer Institute, Inc., Boston, MA (US); Massachusetts Institute of Technology, Cambridge, MA (US); The Brigham and Women's Hospital, Inc., Boston, MA (US)

(72) Inventors: Meromit Singer, Boston, MA (US); Aviv Regev, Cambridge, MA (US); Orit Rozenblatt-Rosen, Cambridge, MA (US); Davide Mangani, Boston, MA (US); Ana Carrizosa Anderson, Boston, MA (US); Vijay K. Kuchroo, Boston, MA (US); Linglin Huang, Boston, MA (US)

(21) Appl. No.: 17/494,062

(22) Filed: Oct. 5, 2021

Related U.S. Application Data
(60) Provisional application No. 63/087,768, filed on Oct. 5, 2020.
(60) Provisional application No. 63/193,367, filed on May 26, 2021.

(51) Int. Cl. A61K 35/17; C07K 16/28; A61K 45/06; A61P 35/00

(52) U.S. Cl. CPC A61K 35/17; C07K 16/2818; A61P 35/00; A61K 45/06; C07K 16/2827

(57) ABSTRACT

The present invention is generally directed to identifying genes and cell types that are correlated with tumor progression in the tumor microenvironment. PENK was identified as a therapeutic target that is positively correlated with tumor time and size. Targeting PENK can enhance anti-tumor immunity. Opioid signaling can be modulated to enhance anti-tumor immunity. The present invention is also generally directed to interacting cells in the tumor microenvironment and using the identified interactions to enhance anti-tumor immunity in cancer. Identified interactions can be modulated using therapeutic agents. Immune cells resistant to suppression can be used for adoptive cell transfer.

### CROSS-REFERENCE TO RELATED APPLICATIONS

This application claims the benefit of U.S. Provisional Application Nos. 63/087,768 filed Oct. 5, 2020, and, 63/193,367 filed May 26, 2021. The entire contents of the above-identified applications are hereby fully incorporated herein by reference.

### SEQUENCE LISTING

This application contains a sequence listing filed in electronic form as an ASCII.txt file entitled BROD-5275US_ST25.txt, created on Sep. 27, 2021 and having a size of 16,057 bytes (16 KB on disk). The content of the sequence listing is incorporated herein in its entirety.

### TECHNICAL FIELD

The subject matter disclosed herein is generally directed to targeting interactions and signaling in the tumor microenvironment to enhance anti-tumor immunity in the treatment of cancer.

### BACKGROUND

Tumor cellular diversity poses both challenges and opportunities for cancer therapy. Various non-malignant cells comprise the tumor microenvironment. The composition of the microenvironment has an important impact on tumorigenesis and in the modulation of treatment responses. Interactions between cells play crucial roles in the tumor microenvironment. The next wave of therapeutic advances in cancer will likely be accelerated by emerging technologies that systematically assess the malignant, microenvironmental, and immunologic states most likely to inform treatment response and resistance. New tools, such as single-cell genomics, have allowed for mapping single cell types in a tissue. A comprehensive cell atlas makes it possible to catalog all cell types and even subtypes of cells in a tissue, and even distinguish different stages of differentiation and cell states, such as immune cell activation. There is a need to further understand communication between cell types during tumor progression.

Citation or identification of any document in this application is not an admission that such a document is available as prior art to the present invention.

### SUMMARY

In certain example embodiments, the invention provides for methods of enhancing anti-tumor immunity based on the upregulated expression of PENK in dysfunctional T cells during tumor progression.

In one aspect, the present invention provides for an isolated immune cell genetically modified ex vivo to decrease or eliminate expression or activity of opioid growth factor receptor (OGFr). In another aspect, the present invention provides for an isolated immune cell genetically modified ex vivo to decrease or eliminate expression or activity of proenkephalin (PENK) or proteolytic derivatives thereof (MENK and LENK). In certain embodiments, the immune cell is a CD8 T cell. In certain embodiments, the CD8 T cell expresses a chimeric antigen receptor (CAR) or T cell receptor (TCR) specific for a tumor antigen. In certain embodiments, the immune cell is a tumor infiltrating lymphocytes (TIL). In another aspect, the present invention provides for a method of enhancing anti-tumor immunity in a subject in need thereof comprising administering to the subject one or more isolated immune cells according to any embodiment herein.

In another aspect, the present invention provides for a method of enhancing anti-tumor immunity in a subject in need thereof comprising administering one or more agents capable of inhibiting opioid growth factor receptor (OGFr) signaling.

In certain embodiments, the one or more agents is a small molecule antagonist of OGFr. In certain embodiments, the small molecule is selected from the group consisting of naloxone, naltrexone, methylnaltrexone (MNTX), and derivatives thereof. In certain embodiments, the small molecule is selective for OGFr and does not antagonize the mu (MOR), delta (DOR), kappa (KOR) or nociceptin (NOR) opioid receptor subtypes. In certain embodiments, the one or more agents comprise a small molecule degrader. In certain embodiments, the method further comprises administering an opioid or opioid signaling agonist in combination with the selective OGFr antagonist.

In certain embodiments, the one or more agents is an antibody to OGFr. In certain embodiments, the one or more agents is an antibody to PENK or PENK-derived polypeptide.

In certain embodiments, the one or more agents is an engineered competitive PENK peptide. In certain embodiments, the engineered competitive PENK peptide is derived PENK or MENK.

In certain embodiments, the one or more agents is an inhibitor of PENK proteolytic cleavage. In certain embodiments, the inhibitor is a furin inhibitor. In certain embodiments, the furin inhibitor is selected from the group consisting of decanoyl-RVKR-chloromethylketone (CMK), hexa-D-arginine (D6R), and phenylacetyl-Arg-Val-Arg-4-amidinobenzylamide. In certain embodiments, the inhibitor is an aminopeptidase inhibitor. In certain embodiments, the aminopeptidase inhibitor is bestatin. In certain embodiments, the inhibitor is a cathepsin L (CTSL) inhibitor. In certain embodiments, the cathepsin L (CTSL) inhibitor is selected from the group consisting of amantadine hydrochloride, teicoplanin, heparin, E64d, MDL28170, and KGP94.

In certain embodiments, the one or more agent comprise a RNAi therapeutic that decreases the expression of PENK.

In certain embodiments, the one or more agents comprise a gene editing system that reduces PENK expression or function. In certain embodiments, the gene editing system is a CRISPR-Cas system, a zinc finger nuclease, a TALEN, or a meganuclease. In certain embodiments, the CRISPR-Cas system is a base editing system or a prime editing system.

In certain embodiments, the agent is administered directly to a tumor in the subject. In certain embodiments, the agent is administered intravenously or intraperitoneally to the subject.

In certain embodiments, the subject is administered an additional immunotherapy. In certain embodiments, the immunotherapy comprises adoptive cell transfer. In certain embodiments, adoptive cell transfer comprises the administration of T cells or natural killer cells that express a CAR (chimeric antigen receptor), T cells expressing a T cell receptor (TCR) specific for a tumor antigen, or tumor infiltrating lymphocytes (TILs). In certain embodiments, the immunotherapy comprises anti-PD-1, anti-CTLA4, anti-PD-L1, anti-TIM3, anti-TIGIT, anti-LAG3, or combinations thereof.

In another aspect, the present invention provides for a method of monitoring tumor progression in a subject in need thereof comprising detecting expression of one or more genes selected from Table 2 or 3. In certain embodiments, the one or more genes is proenkephalin (PENK) or its proteolytic derivatives.

In another aspect, the present invention provides for a method of enhancing anti-tumor immunity in a subject in need thereof comprising administering one or more agents capable of altering expression or activity of one or more genes selected from Table 2 or 3. In certain embodiments, the one or more genes are positively correlated with tumor size or time. In certain embodiments, the one or more genes are upregulated in CD8+PD1+ TIM3+ T cells. In certain embodiments, the one or more genes are upregulated in cluster T_4 or T_7. In certain embodiments, the one or more genes are upregulated in Tregs or cluster T_2. In certain embodiments, the one or more genes are negatively correlated with tumor size or time.

In certain embodiments, the cancer is selected from the group consisting of melanoma, renal cancer, glioma, thyroid cancer, lung cancer, liver cancer, pancreatic cancer, head and neck cancer, stomach cancer, colorectal cancer, urothelial cancer, prostate cancer, testicular cancer, breast cancer, cervical cancer, ovarian cancer and endometrial cancer.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1D—Single cell clusters of B16 tumors. FIG. 1A. tSNE showing single cell clusters of the T cell component of B16 tumors. FIG. 1B. Projection of CD8 and CD4 expression on the T cell tSNE and pie charts showing the percentage of CD4 and CD8 T cells in each cluster. FIG. 1C. tSNE showing single cell clusters of the non-T cell component of B16 tumors. FIG. 1D. Projection of the indicated genes on the non-T cell tSNE.

FIG. 2—Diagram showing the methodology for determining interactions between clusters.

FIG. 3—Heat maps showing the general upregulated ligands and receptors in each cluster.

FIG. 4—Heat maps showing the specific upregulated ligands and receptors in each cluster.

FIG. 5—Table showing interactions between ligand receptor pairs. The 1 cluster indicates the cell clusters that the left gene in the pair is expressed in and r cluster indicates the cell clusters that the right gene in the pair is expressed in. The clusters indicate the cell type, for example, T_X2 Treg; M_X4,6,3 Macrophages; T_X1,4,5,6,9,10 CD8 T cells; M_X9,11,14 malignant cells (see also, FIG. 1A-D).

FIGS. 6A-6C—FIG. 6A. Projection of the indicated genes on the T cell and non-T cell tSNE. FIG. 6B. Projection of the indicated genes on the T cell and non-T cell tSNE. FIG. 6C. Projection of the indicated genes on the T cell and non-T cell tSNE.

FIG. 7—Heat maps showing interacting clusters that express general upregulated ligands and receptors pairs.

FIG. 8—Heat maps showing interacting clusters that express specific upregulated ligands and receptors pairs.

FIG. 9—Network plots showing specific upregulation (30%) interactions for clusters using all ligand-receptor pairs.

FIG. 10—Network plots showing specific upregulation (30%) interactions for clusters using ligand-receptor pairs with a T cell component.

FIGS. 11A-11B—FIG. 11A. Ligand-receptor network. FIG. 11B. Zoom in of indicated ligand-receptor networks.

FIGS. 12A-12B—FIG. 12A. Ligand-receptor network for ITGAV and projection on the tSNE plots. FIG. 12B. Diagram of interacting cells through ITGAV.

FIGS. 13A-13B—FIG. 13A. Ligand-receptor network for SDC4 and projection on the tSNE plots. FIG. 13B. Diagram of interacting cells through SDC4.

FIG. 14—Interaction between ligand-receptor networks for SDC4 and ITGAV.

FIG. 15—Diagram showing the tumor microenvironment.

FIG. 16—Validations of interacting cells using immunohistochemistry.

FIG. 17—Validations of interacting cells using immunohistochemistry.

FIG. 18—Validations of interacting cells using immunohistochemistry.

FIG. 19—Expression of VEGFB in different human tumor types from the cancer genome atlas (TCGA).

FIGS. 20A-20C—FIG. 20A. Projection of the indicated genes on the T cell and non-T cell tSNE. FIG. 20B. Expression of FBLN1 in melanoma patients and the overall survival for patients having high or low expression. FIG. 20C. String data showing interactions between ligands and receptors.

FIG. 21—Graph showing sample information for mice used in the study. Each point represents a mouse at each time point and the size of the tumor in the mouse.

FIG. 22—The number of single T cells and non-T cells from each sample.

FIGS. 23A-23B—FIG. 23A. UMAP analysis showing 10 annotated T cell clusters. FIG. 23B. Expression of cell type markers projected on the UMAP plot.

FIGS. 24A-24B—FIG. 24A. Heat map showing T cell cluster marker genes. FIG. 24B. Graph showing T cell cluster marker genes.

FIG. 25—UMAP analysis showing 12 annotated non-T cell clusters.

FIGS. 26A-26B—FIG. 26A. Heat map showing non-T cell cluster marker genes. FIG. 26B. Graph showing non-T cell cluster marker genes.

FIG. 27—Graph showing the number of up-regulated ligands or receptors in the T cell and non-T cell clusters.

FIG. 28—Graphs showing GSEA for ligand/receptor up-regulation. Points above the line are enriched upregulated for ligand or receptors.

FIG. 29—Heat map showing cluster similarity and the correlation of ligand/receptor average expression profile.

FIGS. 30A-30D—FIG. 30A. Cluster network of interactions between T and non-T cells based on ligand and receptor pairs. FIG. 30B. Cluster network of interactions between T and non-T cell clusters. FIG. 30C. Cluster network of interactions among T cell clusters. FIG. 30D. Cluster network of interactions among non-T cell clusters.

FIG. 31—Gene network. All ligand-receptor pairs that are up-regulated in clusters. Node colored by type of interactions the gene is involved.

FIG. 32—Gene network. Ligand-receptor pairs that are up-regulated in clusters. Only genes and interactions between T and non-T cell clusters are shown. Node colored by T cell cluster the interactions are. Node border colored by if the gene is up-regulated in T, M (non-T) or both types of cell clusters.

FIG. 33—Graphs showing overview of test results examining if the frequencies of different cell types (clusters) change as a function of time after tumor implant and size of tumor. Clusters are positively or negatively associated with time and/or size.

FIG. 34—Graphs showing time vs. cluster size.

FIG. 35—Graphs showing tumor size vs. cluster size.

FIG. 36—Graphs showing tumor size vs. cluster size at different times.

FIGS. 37A-37C—FIG. 37A-B. UMAP plots and violin plots showing expression of Ly6C-monocyte positive (ACE) and negative (CD9) markers. FIG. 37C. Bar graph showing the percentage of Ace+Cd9− cell per cell type.

FIG. 38—FACS analysis validating Ly6C-monocyte increase with time and tumor size.

FIG. 39—Graphs showing data validating Ly6C-monocytes increase with time and tumor size.

FIG. 40—Heat maps of clusters (y) vs. genes (x) showing genes differentially expressed over time and tumor size.

FIGS. 41A-41B—Graphs showing the number of time (A) and tumor size (B) dependent differentially expressed genes in each cluster.

FIG. 42—Graphs showing example time dependent genes having a positive association.

FIG. 43—Graphs showing example time dependent genes having a negative association.

FIG. 44—Graphs showing example size dependent genes having a positive association.

FIG. 45—Graphs showing example size dependent genes having a negative association.

FIG. 46—Graph showing GSEA pathways enriched for time dependent genes.

FIG. 47—Graph showing GSEA pathways enriched for tumor size dependent genes.

FIGS. 48A-48D—Validation of PENK expression in mouse T cell clusters. FIG. 48A. PENK gene counts in cluster T_7 over time (CD8 Pd1+Tim3+). FIG. 48B. PENK gene counts in cluster T_4 over time (proliferating CD8 Pd1+Tim3+). FIG. 48C. PENK gene counts in cluster T_7 (CD8 Pd1+Tim3+) with size. FIG. 48D. PENK gene counts in cluster T_4 (proliferating CD8 Pd1+Tim3+) with size.

FIGS. 49A-49C—Penk is expressed on CD8+PD1+Tim3+ TILs. FIG. 49A. UMAP analysis showing 10 annotated T cell clusters. FIG. 49B. Expression of cell cycle markers projected on the UMAP plot. FIG. 49C. Expression of PENK projected on the UMAP plot.

FIGS. 50A-50D—Proenkephalin (PENK) increases over time and size in CD8⁺ PD1⁺ TIM3⁺ cells. FIG. 50A. PENK expression over time (CD8 Pd1+Tim3+). FIG. 50B. PENK expression with increasing tumor size (CD8 Pd1+Tim3+). FIG. 50C. PENK expression over time (CD8 Pd1+Tim3+). FIG. 50D. PENK expression with increasing tumor size (CD8 Pd1+Tim3+).

FIG. 51—Opioid growth factor receptor is ubiquitously expressed. Expression of OGFR projected on the UMAP plot showing 10 annotated T cell clusters.

FIG. 52—CD8+PD1+ TIM3+ expressing both PENK and OGFR display highest dysfunction signature. Violin plots showing dysfunction signature scores in CD8+PD1+ T cells expressing neither PENK and OGFR, Penk, Ogfr or both.

FIGS. 53A-53B—Delayed B16F10 tumor growth in germline PENK KO mice. FIG. 53A. Graph showing tumor size in wildtype and Penk KO mice. FIG. 53B. Graph showing tumor weight in wildtype and Penk KO mice.

FIGS. 54A-54C—In vitro modulation of Opioid signaling pathway—Penk KO.

FIG. 54A. Graph showing proliferation in wildtype and Penk KO mice. FIG. 54B. Graph showing co-inhibitory receptor expression (PD1, TIM3, CD39) in wildtype and Penk KO mice. FIG. 54C. Graph and FACS showing the percentage of PD1+ TIM3+ cells in wildtype and Penk KO mice.

FIGS. 55A-55D—In vitro modulation of Opioid signaling pathway—MENK stimulation. FIG. 55A. Graph showing proliferation in cells treated with MENK or control. FIG. 55B. Graph showing the percentage of IL2+ cells after treatment with MENK or control. FIG. 55C. Graph showing the percentage of dysfunction markers in cells after treatment with MENK or control. FIG. 55D. Graph and FACS showing the percentage of PD1+ TIM3+ cells after treatment with MENK or control.

FIGS. 56A-56B—Penk-overexpression (OE) cells co-inhibitory receptors expression—(Day8—2 rounds of stimulation). FIG. 56A. Graphs showing co-inhibitory receptor expression (PD1, TIM3, TIGIT, CD39) in Penk-OE cells. FIG. 56B. Graphs showing the percentage of PD1+ TIM3+ cells and PD1+ TIM3+CD39+ cells in Penk-OE cells.

FIG. 57A-57B—In vivo experiment—Penk-OE [Ova]. OT1 CD8 T cells were transduced either with lentivirus control or lentivirus expressing Penk. These cells were adoptively transferred into mice bearing established (Day 6) B16F10 tumors expressing OVA antigen (B16F10-Ova) and tumor growth was followed. FIG. 57A. (left) Graph showing tumor size in mice treated with lentivirus overexpressing PENK in T cells. (right) Graph showing tumor weight in mice treated with lentivirus overexpressing PENK in T cells. FIG. 57B. (left) Graph showing tumor size in mice treated with lentivirus overexpressing PENK in T cells. (right) Graph showing tumor weight in mice treated with lentivirus overexpressing PENK in T cells. NT=no transfer.

FIG. 58—In vivo experiment—Penk-OE—Tumor analyses. Graphs showing hNGFR+ cells in mice treated with lentivirus overexpressing PENK in T cells.

FIG. 59—In vivo experiment—Penk-OE—Tumor analyses—intra-cellular cytokine (ICC) staining. Graphs showing ICC in two mouse tumor models treated with lentivirus overexpressing PENK in T cells.

FIG. 60—Tumor killing in vitro—Ogfr-KO CD8 T cells. OT1 CD8 T cells were transduced on day 1 post-activation with either a lentiviral vector expressing a control sgRNA or an sgRNA targeting OGFR gene. On day 6, cells were harvested, counted and co-cultured with a 50:50 mix of B 16F10 and B16F10-Ova-gfp. Antigen specific killing by CD8 OT1 cells was calculated by checking the live cells ratio between B16F10 and B16F10-Ova-gfp. Further, CD8 OT1 T cells were stained for IFNg and TNFa to assess their effector capacity. (left) Graph showing tumor killing in a tumor—T cell coculture experiment using control T cells and Ogfr-KO T cells. (right) Graphs showing cytokine expressing cells in control T cells and Ogfr-KO T cells.

FIG. 61—In vivo experiment—Penk-OE—Tumor analyses—ICC. Immune cells were isolated from the tumors of mice that received either OTI cells overexpressing penk or control and re-stimulated in vitro with OVA antigen for 4 hours. Antigen-specific responses were quantified by assessing cytolytic capacity (Cd107a+Gzmb+ cells) and capacity to produce effector (Ifng and Tnfa) or pro-survival (IL2) factors. (left) Graphs showing ICC in mouse Ova tumor model treated with lentivirus overexpressing PENK in T cells.

FIG. 62A-62B—In vivo experiment—CD8-specific Penk-KO. FIG. 62A. Mice bearing established tumors expressing the human gp100 antigen (B16-hgp100) were adoptively transferred with either antigen-specific CD8 Pmel control cells or CD8 Pmel Penk KO cells (genetic KO). Tumor growth was followed. FIG. 62B. OT1 CD8 T cells were transduced with either with lentivirus expressing a control sgRNA or lentivirus expressing sgRNA guides targeting the Penk gene for KO. These cells were adoptively transferred into mice bearing established (Day 6) B16F10 tumors expressing OVA antigen (B16F10-Ova) and tumor growth was followed.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to PCT/US2018/053791, PCT/US2018/061812, PCT/US2017/050469, PCT/US2016/059507, PCT/US2016/059487 and PCT/US2016/059463.

All publications, published patent documents, and patent applications cited herein are hereby incorporated by reference to the same extent as though each individual publication, published patent document, or patent application was specifically and individually indicated as being incorporated by reference.

### Overview

Applicants identified genes upregulated in T cells and non-T cells that are correlated with tumor time and size, in particular dysfunctional T cells and suppressive Tregs. During persistent immune activation, such as during uncontrolled tumor growth or chronic infections, subpopulations of immune cells, particularly of CD8+ or CD4+ T cells, become compromised to different extents with respect to their cytokine and/or cytolytic capabilities. Such immune cells, particularly CD8+ or CD4+ T cells, are commonly referred to as “dysfunctional” or as “functionally exhausted” or “exhausted”. As used herein, the term “dysfunctional” or “functional exhaustion” refer to a state of a cell where the cell does not perform its usual function or activity in response to normal input signals, and includes refractivity of immune cells to stimulation, such as stimulation via an activating receptor or a cytokine. Applicants identified that endogenous opioid signaling between cells in the tumor microenvironment is positively correlated with tumor time and size and is specifically upregulated in CD8+TB/13+PD1+ dysfunctional T cells. Applicants identified that PENK is positively associated and upregulated in the most dysfunctional T cells in the tumor environment (CD8+ TIM3+PD1+ T cells). PENK peptides (e.g., MENK, LENK) can be secreted by dysfunctional immune cells to suppress an immune response resulting in a suppressed anti-tumor immune response. As used herein, the term “anti-tumor immunity” refers to an immune response that reduces or prevents proliferation of a cancer cell, such as an immune response induced upon recognition of cancer antigens by immune cells. Anti-tumor immunity can be elicited by effector CD8+ T cells with the ability to simultaneously produce multiple cytokines (polyfunctional CD8+ T cells). Thus, enhancing anti-tumor immunity includes preventing T cells from becoming dysfunctional and enhancing tumor killing by effector CD8+ T cells.

Proenkephalin (PENK), formerly known as proenkephalin A (since proenkephalin B was renamed prodynorphin), is an endogenous opioid polypeptide hormone which, via proteolyic cleavage, produces the enkephalin peptides [Met]enkephalin (MENK), and to a lesser extent, [Leu]enkephalin (LENK). Upon cleavage, each proenkephalin peptide results in the generation of four copies of [Met]enkephalin, two extended copies of [Met]enkephalin, and one copy of [Leu]enkephalin. The opioid growth factor receptor, also known as OGFr, or the ζ-opioid receptor, is a receptor for MENK. The four classical opioid receptor subtypes are the mu (MOR), delta (DOR), kappa (KOR) and nociceptin receptors (NOR) (see, e.g., Waldhoer M, Bartlett S E, Whistler J L. Opioid receptors. Annu Rev Biochem. 2004; 73:953-990; and Stockdale D P, Titunick M B, Biegler J M, et al. Selective opioid growth factor receptor antagonists based on a stilbene isostere. Bioorg Med Chem. 2017; 25(16):4464-4474. doi:10.1016/j.bmc.2017.06.035). These classical opioid receptors belong to the large superfamily of seven transmembrane-spanning (7TM) G protein-coupled receptors (GPCRs). Id. The expression these receptors was not detected in the T and non-T cells. Applicants showed that opioid growth factor receptor (OGFR) is expressed in all T cells. OGFR is not a member of the 7-transmembrane G-protein coupled receptor family and bears no structural homology to the other classic opioid receptor subtypes. MENK is also a potent agonist of the δ-opioid receptor, and to a lesser extent the μ-opioid receptor, with little to no effect on the κ-opioid receptor. Thus, implicating opioid signaling in anti-tumor immunity.

Previous studies concluded that MENK could work as an immune booster (see, e.g., Wang, et al. Methionine enkephalin (MENK) improves lymphocyte subpopulations in human peripheral blood of 50 cancer patients by inhibiting regulatory T cells (Tregs). Hum Vaccin Immunother. 2014; 10(7):1836-1840; Li, et al. Methionine enkephalin (MENK) improved the functions of bone marrow-derived dendritic cells (BMDCs) loaded with antigen. Hum Vaccin Immunother. 2012; 8(9):1236-1242; and Li, et al. Methionine enkephalin (MENK) inhibits tumor growth through regulating CD4+Foxp3+ regulatory T cells (Tregs) in mice. Cancer Biol Ther. 2015; 16(3):450-459). In contrast, Applicants demonstrate herein that PENK plays a role in suppressing anti-tumor immunity, specifically by dysfunctional T cells. Applicants show that tumor growth is suppressed in PENK knockout mice, because anti-tumor immunity is increased and dysfunction is decreased. Applicants also show that PENK overexpression increases tumor growth because anti-tumor immunity is decreased and dysfunction is increased. Applicants also show that MENK stimulation increases coinhibitory receptors on T cells, thus increasing dysfunction. Applicants also show that adoptive transfer of CD8 T cells knocked out for the receptor OGFr in a mouse tumor model had increased tumor killing because the cells are less responsive to PENK peptides. Thus, the discovery provides new targets for enhancing anti-tumor immunity. PENK or OGFR expression or activity can be targeted. PENK peptides can be targeted. T cells can also be generated that are resistant to dysfunction by altering opioid signaling in the cells. The discovery also has implications in the administration of pain medicine to subjects undergoing an immunotherapy or cancer treatment in general. Pain medication may induce T cell dysfunction and reduce anti-tumor immunity, especially when a subject is being treated with an immunotherapy.

Applicants also identified genes that could be signaling molecules secreted from T and non T cells that bind to receptors expressed on other immune cells.

### Method of Enhancing Anti-Tumor Immunity by Inhibiting Ogfr Signaling

In one aspect, embodiments disclosed herein provide methods for enhancing anti-tumor immunity in a subject in need thereof comprising administering one or more agents capable of inhibiting opioid growth factor receptor (OGFr) signaling. In one example embodiment, the one or more agents comprise a small molecule that antagonistically binds OGFr natural ligand binding. In another example embodiment, the one or more agents comprise an antibody to OGFR, PENK, or a PENK-derived peptide. In another example embodiment, the one or more agents comprises an aptamer that binds OGFR, or PENK or PENK-derived polypeptides In another example embodiment, the one or more agents may comprise an engineered PENK or PENK-derived peptide that competitively binds with naturally occurring PENK or PENK-derived peptides for binding to OGFr. In another example embodiment, the one or more agents may comprise one or more peptidase inhibitors that interfere with proteolytic cleavage of PENK into biologically active peptides (e.g. MENK and LENK). In another example embodiment, the one or more agents may comprise one or more small molecule degraders. In another example embodiment, the one or more agents may comprise a RNAi based therapeutic that reduces PENK expression. In another example embodiment, the one or more agents may comprise a gene editing system that reduces or modifies expression of PENK in tumor cells.

The need for enhancing anti-tumor immunity may apply to any cancer type. The cancer may include, without limitation, liquid tumors such as leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, or multiple myeloma.

The cancer may include, without limitation, solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumour including serous tumour, endometrioid tumor and mucinous cystadenocarcinoma, sex-cord-stromal tumor), prostate cancer, liver and bile duct carcinoma (e.g., hepatocelluar carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, medullablastoma, astrocytoma, medulloblastoma, craniopharyngioma, ependymoma, pinealoma, hemangioblastoma, acoustic neuroma, oligodenroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases.

### 1. Small Molecule Antagonist of OGFr Binding or Activity

In one example embodiment, a method for enhancing anti-tumor immunity comprises administering one or more small molecule antagonists of OGFR binding or activity. As used herein “Opioid Growth Factor Receptor (OGFR) antagonist” refers to any molecule that inhibits, suppresses or causes the cessation of at least one OGFR-mediated biological activity. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking an enzyme active site or activating a receptor by binding to a ligand binding site).

In one example embodiment, the small molecule is a nonselective antagonist. Nonselective opioid signaling antagonists can inhibit OGFR (see, e.g., U.S. Pat. No. 10,208,306) in addition to the mu (MOR), delta (DOR), kappa (KOR) and nociceptin receptors (NOR). For example, naloxone is a nonselective antagonist with a relative affinity of μ>δ>κ (see, e.g., Malenka R C, Nestler E J, Hyman S E (2009). Sydor A, Brown R Y (ed.). Molecular Neuropharmacology: A Foundation for Clinical Neuroscience (2nd ed.). New York: McGraw-Hill Medical. pp. 190-191, 287). In one example embodiment, the OGFr antagonist is selected from naloxone, naltrexone, methylnaltrexone (MNTX), or a functional derivative thereof. As used herein, a “functional derivative” refers to a derivative or analog that is structurally and functionally analogous to the originating molecule. Examples of naltrexone and naloxone functional derivatives include salt forms, e.g., naloxone hydrochloride dihydrate or naltrexone hydrochloride. Additional examples of naltrexone and naloxone functional derivatives suitable for use in the present methods include naltrexone and naloxone analogs disclosed in U.S. Patent Application Publication No. 2007/0197573 A1, U.S. Pat. No. 6,713,488. Naloxone, sold under the brand name Narcan among others, is a medication used to block the effects of opioids, in particular, to treat heroin and other opiate overdoses. Naltrexone, sold under the brand names ReVia and Vivitrol among others, is a medication primarily used to manage alcohol or opioid dependence.

In one example embodiment, an OGFr binding antagonist is derived from oxymorphone and binds to the OGFr, which includes naloxone, naltrexone, nalorphine, naloxonazine, levallorphan, nalmefene, cyprodime, cyclorphan, cyclazodne, oxilorphan, LY113878, MR2266, diprenorphine, WIN 44,441-3, naltindole, or norbinaltorphimine.

In still another embodiment, an OGFr binding antagonist is derived from trans-3,4-dimethyl-4-phenylpiperidine and binds to the OGFr, which includes LY99335, LY25506, LY117413, or LY255582.

One type of small molecule applicable to the present invention is a degrader molecule (see, e.g., Ding, et al., Emerging New Concepts of Degrader Technologies, Trends Pharmacol Sci. 2020 July; 41(7):464-474). The terms “degrader” and “degrader molecule” refer to all compounds capable of specifically targeting a protein for degradation (e.g., ATTEC, AUTAC, LYTAC, or PROTAC, reviewed in Ding, et al. 2020). Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810). In certain embodiments, LYTACs are particularly advantageous for cell surface proteins as described herein.

1a.—Use of Opioid or Opioid Signaling Antagonist in Combination with Opioid Pain Medication

In one example embodiment, the small molecule is a selective OGFr antagonist. Nonselective opioid signaling antagonists can target more than one opioid receptor. Thus, administration of such an antagonist may block receptors required to alleviate pain and may further block the effects of pain medication. In preferred embodiments, treatment can be performed with as little pain for the subject as possible, such as when treating cancers where many patients are being treated for chronic pain. As indicated herein, the administration of pain medicine may suppress an anti-tumor immune response by activating opioid receptors, in particular receptors activated by PENK peptides. In certain embodiments, the small molecule is selective for OGFr and does not antagonize the mu (MOR), delta (DOR), kappa (KOR) or nociceptin (NOR) opioid receptor subtypes. In one example embodiment, selective antagonists of OGFR may be administered to a subject in combination with opioid pain medicine, such that pain is reduced without suppressing an anti-tumor immune response.

Selective antagonists of OGFr have been described (see, e.g., Stockdale, David P et al. “Selective opioid growth factor receptor antagonists based on a stilbene isostere.” Bioorganic & medicinal chemistry vol. 25, 16 (2017): 4464-4474. doi:10.1016/j.bmc.2017.06.035).

In example embodiments, “low-dose” naltrexone therapy is used as an OGFr selective treatment, such as about 4.5 mg naltrexone/day (see, e.g., Smith J P, Stock H, Bingaman S, Mauger D, Rogosnitzky M, Zagon I S. Low-dose naltrexone therapy improves active Crohn's disease. Am J Gastroenterol. 2007; 102(4):820-828).

### 2. Antibodies

In one example embodiment, a method of enhancing an anti-tumor response comprises administering one or more antibodies. The antibodies may bind to OGFR, PENK, or MENK. Antibodies recognizing MENK have been generated and are commercially available (see, e.g., U.S. Pat. No. 8,013,123B2; and anti-MENK ImmunoStar, New Richmond, Wis., USA). Prior to the present study one skilled in the art would have no motivation to target MENK with antibodies, as previous studies suggest administering MENK to enhance an immune response (see, e.g., Wang, et al. Methionine enkephalin (MENK) improves lymphocyte subpopulations in human peripheral blood of 50 cancer patients by inhibiting regulatory T cells (Tregs). Hum Vaccin Immunother. 2014; 10(7):1836-1840; Li, et al. Methionine enkephalin (MENK) improved the functions of bone marrow-derived dendritic cells (BMDCs) loaded with antigen. Hum Vaccin Immunother. 2012; 8(9):1236-1242; and Li, et al. Methionine enkephalin (MENK) inhibits tumor growth through regulating CD4+Foxp3+ regulatory T cells (Tregs) in mice. Cancer Biol Ther. 2015; 16(3):450-459). MENK is secreted outside of the cell and is easily accessible by antibodies before it is internalized (see, e.g., Cheng F, McLaughlin P J, Banks W A, Zagon I S. Internalization of the opioid growth factor, [Mets]-enkephalin, is dependent on clathrin-mediated endocytosis for downregulation of cell proliferation. Am J Physiol Regul Integr Comp Physiol. 2010; 299(3):R774-R785). One skilled in the art could easily generate a therapeutic antibody to block MENK binding to OGFr once provided with the function disclosed herein (see, e.g., Lu, R M., Hwang, Y C., Liu, I J. et al. Development of therapeutic antibodies for the treatment of diseases. J Biomed Sci 27, 1 (2020)).

In certain embodiments, the one or more agents is an antibody. The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH} and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g. the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, IgM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins—harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant cross-reactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly cross-react with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, VH and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the C_{H}1 domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

In certain embodiments, the one or more therapeutic agents can be bi-specific antigen-binding constructs, e.g., bi-specific antibodies (bsAb) or BiTEs, that bind two antigens (see, e.g., Suurs et al., A review of bispecific antibodies and antibody constructs in oncology and clinical challenges. Pharmacol Ther. 2019 September; 201:103-119; and Huehls, et al., Bispecific T cell engagers for cancer immunotherapy. Immunol Cell Biol. 2015 March; 93(3): 290-296). The bi-specific antigen-binding construct includes two antigen-binding polypeptide constructs, e.g., antigen binding domains, wherein at least one polypeptide construct specifically binds to a surface protein. In some embodiments, the antigen-binding construct is derived from known antibodies or antigen-binding constructs. In some embodiments, the antigen-binding polypeptide constructs comprise two antigen binding domains that comprise antibody fragments. In some embodiments, the first antigen binding domain and second antigen binding domain each independently comprises an antibody fragment selected from the group of: an scFv, a Fab, and an Fc domain. The antibody fragments may be the same format or different formats from each other. For example, in some embodiments, the antigen-binding polypeptide constructs comprise a first antigen binding domain comprising an scFv and a second antigen binding domain comprising a Fab. In some embodiments, the antigen-binding polypeptide constructs comprise a first antigen binding domain and a second antigen binding domain, wherein both antigen binding domains comprise an scFv. In some embodiments, the first and second antigen binding domains each comprise a Fab. In some embodiments, the first and second antigen binding domains each comprise an Fc domain. Any combination of antibody formats is suitable for the bi-specific antibody constructs disclosed herein.

### 3. Aptamers

In certain embodiments, the one or more OGFR antagonists may be an aptamer that competitively binds to OGFR or binds to PENK or PENK-derived polypeptides. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH₂), 2′-fluoro (2′-F), and/or 2′-O-methyl (2′-OMe) substituents. Modifications of aptamers may also include, modifications at exocyclic amines, substitution of 4-thiouridine, substitution of 5-bromo or 5-iodo-uracil; backbone modifications, phosphonothioate or allyl phosphate modifications, methylations, and unusual base-pairing combinations such as the isobases isocytidine and isoguanosine. Modifications can also include 3′ and 5′ modifications such as capping. As used herein, the term phosphonothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, 0-allyl, S-alkyl, S-allyl, or halo group. Methods of synthesis of 2′-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments aptamers are chosen from a library of aptamers. Such libraries include, but are not limited to those described in Rohloff et al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see, e.g., SomaLogic, Inc., Boulder, Colo.). In certain embodiments, the present invention may utilize any aptamer containing any modification as described herein.

### 4. Engineered Competitive PENK Peptides

In one example embodiment, a method of enhancing an anti-tumor immune response comprises administering one or more an engineered competitive PENK or PENK-derived peptides. The competitive peptide may compete with the met-5 ligand for binding to the OGFR on the surface of the nuclear membrane, thereby interfering with, blocking or otherwise preventing the binding of the met5-ligand to the OGFr, without triggering the downstream signaling that would otherwise be induced by the binding of the met-5 ligand to the OGFr. Alternatively, an OGFr binding antagonist may bind to or sequester pro-enkephalin (PENK) or the met-5 ligand with sufficient affinity and specificity to substantially interfere with, block or otherwise prevent binding of met-5 ligand to the OGFr, thereby inhibiting, suppressing or causing the cessation of at least one OGFr-mediated biological activity. Generally, OGFr binding antagonists may be large molecules (e.g., antibodies) or small molecules and may be a polypeptide, nucleic acid, or a synthetic small molecule compound. OGFr binding antagonists may be identified with any in vitro assay readily selected by one of skill in the art.

In one example embodiment, an OGFr binding antagonist is derived from the met5-enkephalin or leu-enkephalin peptides, binds to the OGFr, and minimally includes the following amino acid sequences as a means of targeting the OGFR: Tyr-Gly-Gly-Phe-Met (SEQ ID NO: 1) for those derived from met5-enkephalin or Tyr-Gly-Gly-Phe-Leu (SEQ ID NO: 2) for those derived from the leu-enkephalin.

In still another example embodiment, an OGFR binding antagonist is derived from the peptide antagonist 101174864 (N,N-diallyl-Tyr-Aib-Aib-Phe-Leu-OH, SEQ ID NO: 3; Aib=aminoisobutyticacid) or somatostatin analog CTP (D-Phe-Cys-Tyr-D-Trp-Lys-Thr-Pen-Thr-NH.sub.2, SEQ ID NO: 4).

In another example embodiment, the OGFR antagonist, instead of being an OGFR binding antagonist, is a molecule that disrupts the nuclear localization sequence found within OGFR: QSALDYFMFAVRCRHQRRQLVHFAWEHFRPRCKFVWGPQDKLRRFKPSSL (SEQ ID NO: 5).

### 5. Inhibitors of PENK Proteolytic Cleavage

In one example embodiment, a method of enhancing anti-tumor activity comprises administering an inhibitor that prevents enzymatic cleavage of PENK into biologically active peptides.

In one example embodiment, the inhibitor is an inhibitor of furin (see, e.g., Cheng Y W, Chao T L, Li C L, et al. Furin Inhibitors Block SARS-CoV-2 Spike Protein Cleavage to Suppress Virus Production and Cytopathic Effects. Cell Rep. 2020; 33(2):108254; Imran M, Saleemi M K, Chen Z, et al. Decanoyl-Arg-Val-Lys-Arg-Chloromethylketone: An Antiviral Compound That Acts against Flaviviruses through the Inhibition of Furin-Mediated prM Cleavage. Viruses. 2019; 11(11):1011; Couture F, Kwiatkowska A, Dory Y L, Day R. Therapeutic uses of furin and its inhibitors: a patent review. Expert Opin Ther Pat. 2015; 25(4):379-396; and WO2013029182A1).

In certain embodiments, the therapeutic agent is an inhibitor of aminopeptidase B (encoded for by the RNPEP gene). In certain embodiments, the inhibitor is ubenimex (bestatin), which is a specific inhibitor of aminopeptidase B and leucine aminopeptidase. Bestatin does not show any inhibition of aminopeptidase A, trypsin, chymotrypsin, elastase, papain, pepsin or thermolysin. Bestatin at 100 pg/ml showed no antibacterial and no antifungal activities. It has low toxicity with no death after intraperitoneal injection of 300 mg/kg to mice (see, e.g., Umezawa H, Aoyagi T, Suda H, Hamada M, Takeuchi T, Bestatin, an inhibitor of aminopeptidase B, produced by actinomycetes, J Antibiot (Tokyo). 1976 January; 29(1):97-9).

In certain embodiments, the therapeutic agent is an inhibitor of CTSL (cathepsin L) (see, e.g., Sudhan D R, Rabaglino M B, Wood C E, Siemann D W. Cathepsin L in tumor angiogenesis and its therapeutic intervention by the small molecule inhibitor KGP94. Clin Exp Metastasis. 2016; 33(5):461-473; and Gomes C P, Fernandes D E, Casimiro F, et al. Cathepsin L in COVID-19: From Pharmacological Evidences to Genetics. Front Cell Infect Microbiol. 2020; 10:589505). In certain embodiments, the inhibitor is MDL28170 (also known as calpain inhibitor III, or Z-Val-Phe-CHO), which is an inhibitor of cytosolic calpains (see, e.g., Simmons G, Gosalia D N, Rennekamp A J, Reeves J D, Diamond S L, Bates P. Inhibitors of cathepsin L prevent severe acute respiratory syndrome coronavirus entry. Proc Natl Acad Sci USA. 2005; 102(33):11876-11881).

### 6. Inhibition of PENK Expression by RNAi

In one example embodiment, a method of enhancing anti-tumor activity comprises administering a RNAi therapeutic to reduce expression of PENK (“target sequence”). A RNAi therapeutic comprises a polynucleotide that is complementary to a portion of the target sequence mRNA, generally ranging in size from 15 to 50 base pairs. RNAi modalities may include miRNA and siRNA. The RNAi modality may also be in the form a pre-miRNA which is processed by Dicer to form a miRNA. Likewise, the RNAi modality may be in the form of a dsRNA or shRNA which is processed by Dicer to form a siRNA. RNAi modalities may also be derived from endogenous microRNA. The RNAi polynucleotide may comprise one or more modifications to suppress innate immune activation, enhance activity and specificity, and reduce off-target induced toxicity. The RNAi therapeutic may further comprise a delivery platform for delivery of the RNAi polynucleotide.

The RNAi modalities used herein may be used to achieve gene silencing of PENK expression. As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

### 6a.—Example Polynucleotide Sequences

The following sections provide example target sequences to which RNAi polynucleotides may be designed to hybridize to and induce RNAi mediated knockdown of target gene expression. Exemplary sequences for PENK are identified by the following NCBI accession numbers NM_001135690.3 and NP_001129162.1. PENK is located on human chromosome 8, accession No. NC_000008.11 from position 56440957 to 56446641.

All gene name symbols as used throughout the specification refer to the gene as commonly known in the art. The examples described herein that refer to gene names are to be understood to encompass human genes, as well as genes in any other organism (e.g., homologous, orthologous genes). The term, homolog, may apply to the relationship between genes separated by the event of speciation (e.g., ortholog). Orthologs are genes in different species that evolved from a common ancestral gene by speciation. Normally, orthologs retain the same function in the course of evolution. Gene symbols may be those referred to by the HUGO Gene Nomenclature Committee (HGNC) or National Center for Biotechnology Information (NCBI). Any reference to the gene symbol is a reference made to the entire gene or variants of the gene. Reference to a gene encompasses the gene product (e.g., protein encoded for by the gene).

6b.—Example siRNA Embodiments

In one example embodiment, the RNAi modality is a siRNA. As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

6c.—Example shRNA Embodiments

In one example embodiment, the RNAi modality is a shRNA. As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

6d.—Example microRNA Embodiments

In one example embodiment, the RNAi modality is engineered microRNA derived from an endogenous. The terms “microRNA” or “miRNA”, used interchangeably herein, are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

6e.—Example dsRNA Embodiments

In one example embodiment, the RNAi modality is a dsRNA. As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### 6f.—Example RNAi Configurations

In an embodiment, a single-stranded RNAi molecule disclosed herein has a single-stranded oligonucleotide structure and mediates RNA interference against a target RNA (e.g., PENK). A single-stranded PENK RNAi agent comprises: (a) a nucleic acid portion comprising a first nucleotide portion (N1) and a second nucleotide portion (N2), wherein said nucleic acid portion comprises at least 8 nucleotides that can base pair with a target RNA, and wherein the total number of nucleotides within the nucleic acid portion is from 8 to 26 nucleotides; and, (b) an internal spacer portion comprising at least a first non-nucleotide spacer portion (S1) that covalently links the first and second nucleotide portions. The first and second nucleotide portions are not self-complementary. The total number of nucleotides of a single-stranded PENK RNAi agent disclosed herein (e.g., 8 to 26) is distributed between the nucleotide portions of the RNAi molecule, wherein each nucleotide portion contains at least one nucleotide.

In one embodiment, the nucleic acid portion of a single-stranded PENK RNAi agent disclosed herein contains two nucleotide portions, referred to as the first nucleotide portion (N1) and the second nucleotide portion (N2). The first and second nucleotide portions of a PENK RNAi agent disclosed herein are covalently attached to a non-nucleotide spacer portion of the molecule. In another embodiment, the nucleic acid portion of the PENK single-stranded RNAi agent disclosed herein contains more than one nucleotide portion (e.g., 3, 4, or 5, referred to as third (N3), fourth (N4) or fifth (N5) nucleotide portions, respectively).

In one embodiment, the internal spacer portion of a single-stranded PENK RNAi agent disclosed herein contains only one non-nucleotide spacer portion, referred to as the first non-nucleotide spacer portion (S1). The first non-nucleotide spacer portion (S1) of a PENK RNAi agent disclosed herein is covalently attached to two nucleotides and/or non-nucleotide substitutes, each located within a distinct nucleotide portion of the single-stranded molecule. In another embodiment, the internal spacer portion of a single-stranded PENK RNAi agent disclosed herein contains more than one non-nucleotide spacer portion (e.g., 2, 3, or 4, referred to as second (S2), third (S3) or fourth (S4) non-nucleotide spacer portions, respectively).

A single-stranded PENK RNAi agent disclosed herein can comprise substitutions, chemically modified nucleotides, and non-nucleotides, including substitutions or modifications in the backbone, sugars, bases, or nucleosides. In certain embodiments, the use of substituted or modified single-stranded PENK RNAi agents can enable achievement of a given therapeutic effect at a lower dose since these molecules may be designed to have an increased half-life in a subject or biological samples (e.g., blood). Furthermore, certain substitutions or modifications can be used to improve the bioavailability of single-stranded PENK RNAi agents by targeting particular cells or tissues or improving cellular uptake of the single-stranded PENK RNAi agents.

In an embodiment, the internal spacer portion of a single-stranded PENK RNAi agent can comprise one or more non-nucleotide spacer portions. A non-nucleotide spacer portion can include any aliphatic or aromatic chemical group that can be further substituted, wherein said spacer portion does not contain a nucleotide. The spacer portion can be substituted with a chemical moiety that provides additional functionality to a single-stranded PENK RNAi agent. For example, a non-nucleotide spacer portion can be substituted with a moiety that binds specifically to a target molecule of interest or facilitates/enhances cellular delivery of the molecule. In one embodiment, a non-nucleotide spacer portion includes an alkyl, alkenyl or alkynyl chain of preferably 1 to 20 carbons that can be optionally substituted.

The single-stranded PENK RNAi molecules disclosed herein are useful agents, which can be used in methods for a variety of therapeutic, diagnostic, genetic engineering, and pharmacological applications. Thus, embodiments of the present disclosure further include methods comprising using a single-stranded PENK RNAi agent and methods for inhibiting PENK expression of one or more corresponding target mRNAs to enhance anti-tumor immunity in a cell or organism. Further, this disclosure provides methods and PENK RNAi agents for treating a subject, by enhancing anti-tumor immunity in a subject in need thereof, including a human cell, tissue, individual or subject.

### 6g.—Modifications

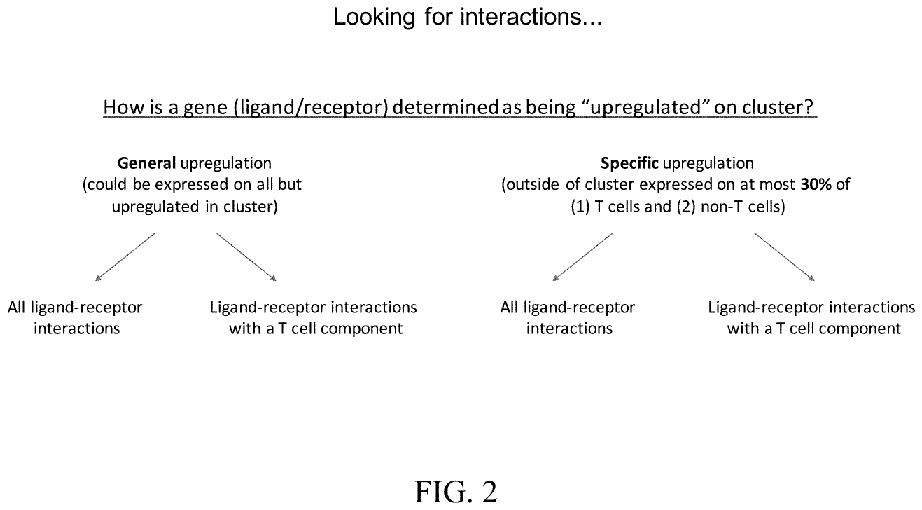

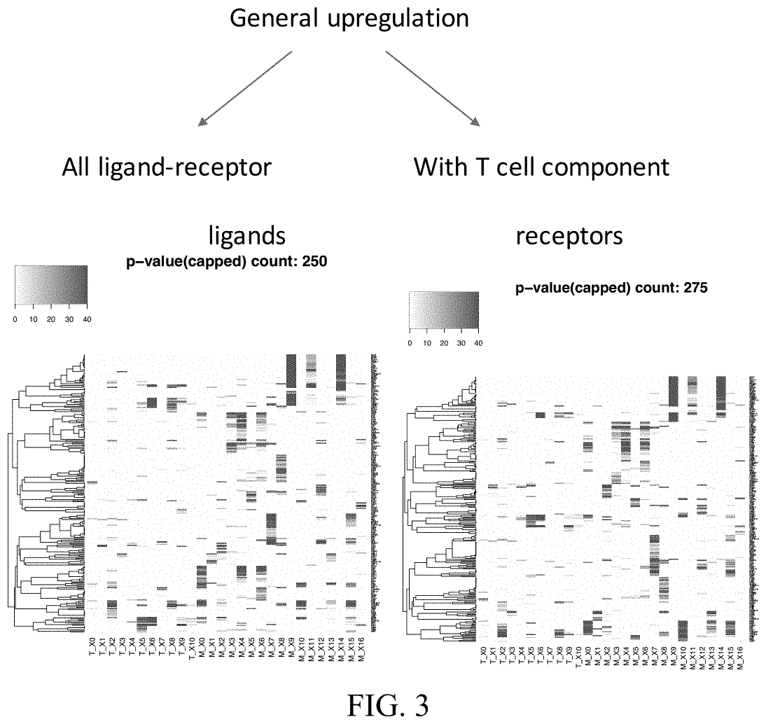

The RNAi modalities described above may comprise one or more modifications including, but not limited to, base modification, ribose modifications, and phosphate modifications. Example base modifications may include 2′-O-methyl, 2′ 0-methoxyethyl, 2′-arabinoo-fluoro, 2′-O-benzyl, 2′-O-methyl-4-pyridine, locked nucleic acid (LNA), (S)-cEt-BNA, tricyclo-DNA, PMO, unlocked nucleic acid, and glycol nucleic acid. Phosphate modifications include phophoorothioate (PS, Rp isomer, and PS, Sp isomer), phosphorodithioate, methylphosphonate, methoxypropyl-phosphonate, 5′-(E)-vinylphophonate, 5′-Methyl Phosphonate, (S)-5′-C-methyl with phosphate, 5′-phosphorothioate, and peptide nucleic acid. Base modifications may include pseudouridine, 2′-thiouridine, N6′-methyladenosine, 5′-methylcytidine, 5′-fluoro-2′-deoxyuridine, N-ethylpiperidine 7′-EAA triazole modified adenine, N-ethylpiperidine 6′-triazole modified adenine, 6′-phenylpyrrolo-cytosinie, 2′,4′-difluorotoluly ribonucleoside, and 5′-nitroindole. A summary of modifications and example locations within a RNAi polynucleotide for each modification are describe in Hu et al. “Therapeutic siRNA: state of the art” Signal Transduction and Targeted Therapy 5, Article number 100 (2020), particularly FIGS. 2 and 3, which are incorporated herein by reference.

### 6h.—Delivery Platforms

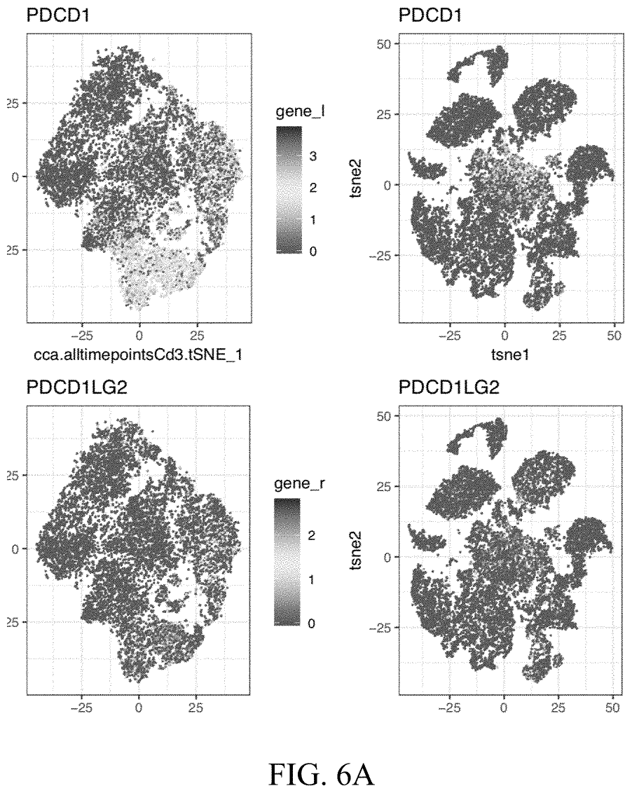

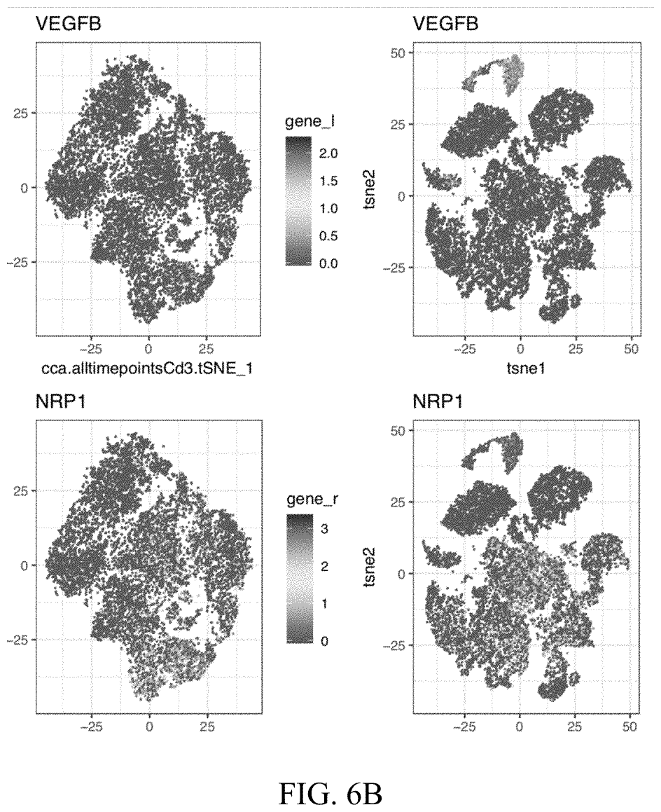

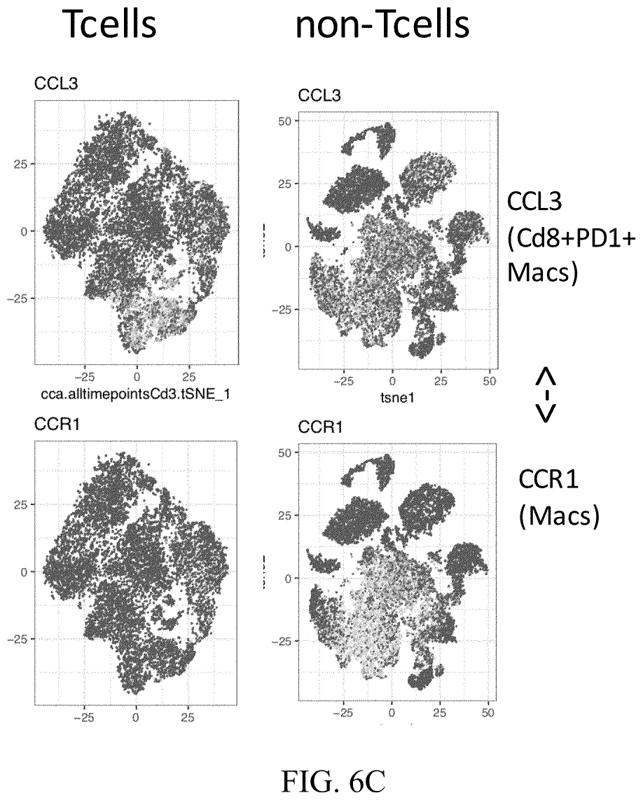

While the RNAi polynucleotides described above may be delivered as naked RNA (with or without modification) in certain example embodiments, the RNAi therapeutic may further comprise a delivery platform. Example delivery planforms include, but are not limited to liposomes, conjugates, peptides, exosomes, polymers, dendrimers, and inorganic nanoparticles. Example liposomes include Dlin-DMA, Dlin-MC3-DMA, and EnCore. Example conjugates include GalNAc, cholesterol, and RGD. Example polymers include cyclodextrin, PBAVE, PEI, and PLGA. Example peptides include DPC2.0 (MLP), and PNP. Example delivery platforms are described in Hu et al. “Therapeutic siRNA: state of the art” Signal Transduction and Targeted Therapy 5, Article number 100 (2020), particularly pages 11-20 and FIG. 6, which are incorporated herein by reference.

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. There are no limitations regarding the type of vector that can be used. The vector can be a cloning vector, suitable for propagation and for obtaining polynucleotides, gene constructs or expression vectors incorporated to several heterologous organisms. Suitable vectors include eukaryotic expression vectors based on viral vectors (e.g. adenoviruses, adeno-associated viruses as well as retroviruses and lentiviruses), as well as non-viral vectors such as plasmids. Exemplary therapeutic delivery vectors of RNAi including viruses are described in Nguyen et al. “RNAi therapeutics: An update on delivery” (2008). Current Opinion in Molecular Therapeutics 10(2): 158-167. Exemplary RNAi delivery vectors from a variety of viruses including, but not limited to, adenovirus (Ad), adeno-associated virus (AAV), retroviruses, et al. are described in Lundstrom, K. “Viral Vectors Applied for RNAi-Based Antiviral Therapy” Viruses (2020) 12, 924 doi:10:3390/v12092924 (14 pages), particularly on pages 3 and 4, which are herein incorporated by reference. Exemplary viral vectors using alphaviruses, flaviviruses, measles viruses and rhabdoviruses are described in Lundstrom, K. “Self-Amplifying RNA Viruses as RNA Vaccines” 21, 5130 (2020); doi:10.3390/ijms21145130 (29 pages), particularly the viral vectors listed on page 6, which are herein incorporated by reference.

In one example embodiment, the vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., episomal mammalian vectors). Other vectors (e.g., non-episomal mammalian vectors) are integrated into the genome of a host cell upon introduction into the host cell, and thereby are replicated along with the host genome.

In one example embodiment, the vector is a “plasmid,” which refers to a circular double stranded DNA loop into which additional DNA segments can be inserted, such as by standard molecular cloning techniques.

In one example embodiment, RNAi molecules are delivered via liposomes. The RNAi molecules may be modified.

### 7. Inhibition or Modification of PENK Expression Using Gene Editing Systems

In one example embodiment, a gene editing system is used to reduce the expression or activity of PENK (“target sequence”) in tumor cells. In one example embodiment, a programmable nuclease is used to make one or more insertions or deletions the target sequence that results in reduced expression of the target sequence. In another example embodiment, a programmable nuclease, in combination with a donor template, is used to replace a target sequence with either a non-functional variant of the target sequence or a modified target sequence that results in expression of a gene product of reduced activity. In one example embodiment, a catalytically inactive programmable nuclease is used to recruit a functional domain (e.g., repressor domain) to the target gene to reduce expression. In one example embodiment, the gene editing system is a base editing system. In one example embodiment, the base editing system is a DNA base editing system used to make one or more base or base pair edits to the target sequence that reduce target sequence expression. In one example embodiment, the base editing system is a RNA base editing system used to modify mRNA expressed from the target gene to reduce protein function, for example, by modifying one or more post-translation modification sites encoded by the mRNA. In another example embodiment, the gene editing system is a prime editing system. A prime editing system may be used to edit DNA like a base editing system. A prime editing system may also be used to replace all or a portion of the target genes to produce a non-functional variant or expression of a gene product with reduced activity.

### 7a.—CRISPR-Cas

In one example embodiment, the gene editing system is a CRISPR-Cas system. The CRISPR-Cas systems comprises a Cas polypeptide and a guide sequence, wherein the guide sequence is capable of forming a CRISPR-Cas complex with the Cas polypeptide and directing site-specific binding of the CRISPR-Cas sequence to a target sequence. The Cas polypeptide may induce a double- or single-stranded break at a designated site in the target sequence. The site of CRISPR-Cas cleavage, for most CRISPR-Cas systems, is dictated by distance from a protospacer-adjacent motif (PAM), discussed in further detail below. Accordingly, a guide sequence may be selected to direct the CRISPR-Cas system to induce cleavage at a desired target site at or near the one or more variants.

7(a)(1)—NHEJ-Based Editing

In one example embodiment, the CRISPR-Cas system is used to introduce one or more insertions or deletions that reduces or inhibits PENK expression or activity. More than one guide sequence may be selected to insert multiple insertion, deletions, or combination thereof. Likewise, more than one Cas protein type may be used, for example, to maximize targets sites adjacent to different PAMs. In one example embodiment, a guide sequence is selected that directs the CRISPR-Cas system to make one or more insertions or deletions that reduces PENK expression. In one example embodiment, the CRISPR-Cas system, may be to generate an insertion or deletion that introduces a pre-mature stop codon in the genomic DNA sequence encoding PENK. In another example embodiment, the CRISPR-Cas system may be used to generate an insertion or deletion that removes or interrupts a cleavage recognition site of PENK to prevent generation of PENK-derived signaling peptides. In another example embodiment, the CRISPR-Cas system may be used to generate an insertion or deletion that removes or interrupts a regulatory sequence in the PENK gene, such as a promoter or enhancer sequence.

7(a)(2)—HDR Template Based Editing

In one example embodiment, a donor template is provided to replace a genomic sequence comprising one or more variants that reduce PENK expression. A donor template may comprise an insertion sequence flanked by two homology regions. The insertion sequence comprises an edited sequence to be inserted in place of the target sequence (e.g. a portion of genomic DNA comprising the one or more variants). The homology regions comprise sequences that are homologous to the genomic DNA strands at the site of the CRISPR-Cas induced double-strand break. Cellular HDR mechanisms then facilitate insertion of the insertion sequence at the site of the DSB. In one example embodiment, the donor template may introduce a pre-mature stop codon into the genomic DNA sequence encoding PENK. In another example embodiment, the donor template may be used to insert a fragment into the genomic DNA sequence encoding PENK that renders the gene or gene product non-functional.

The donor template may include a sequence which results in a change in sequence of 1, 2, 3, 4, 5, 6, 7, 8, 9, 10, 11, 12 or more nucleotides of the target sequence.

A donor template may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 1 10+/−10, 120+/−10, 130+/−10, 140+/−10, 150+/−10, 160+/−10, 170+/−10, 1 80+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 1 10+/−20, 120+/−20, 130+/−20, 140+/−20, I 50+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

The homology regions of the donor template may be complementary to a portion of a polynucleotide comprising the target sequence. When optimally aligned, a donor template might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The donor template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

Homology arms of the donor template may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

In one example embodiment, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

The donor template may further comprise a marker. Such a marker may make it easy to screen for targeted integrations. Examples of suitable markers include restriction sites, fluorescent proteins, or selectable markers. The donor template of the disclosure can be constructed using recombinant techniques (see, for example, Sambrook et al., 2001 and Ausubel et al., 1996).

In one example embodiment, a donor template is a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

7(a)(3)—Class 1 Systems

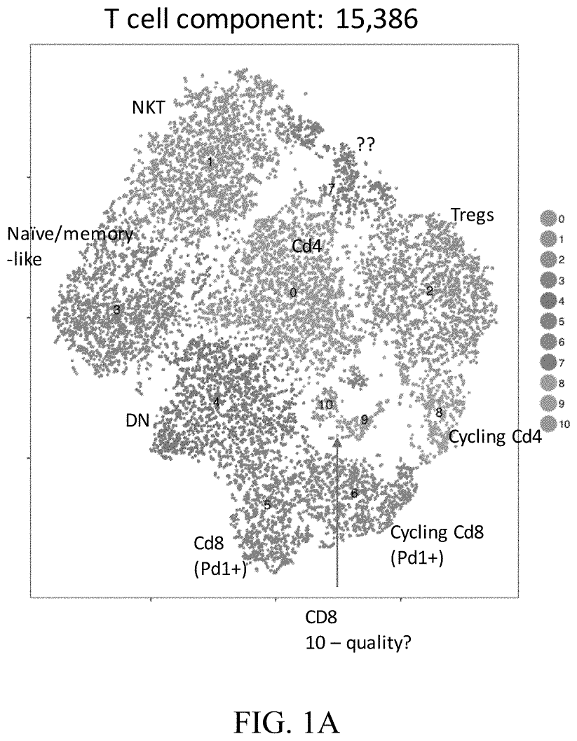

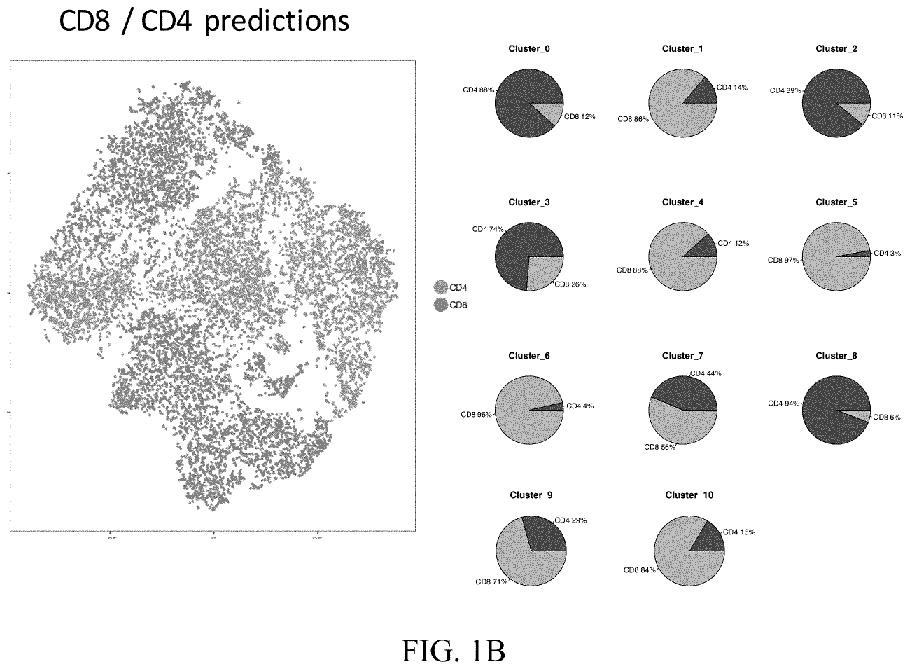

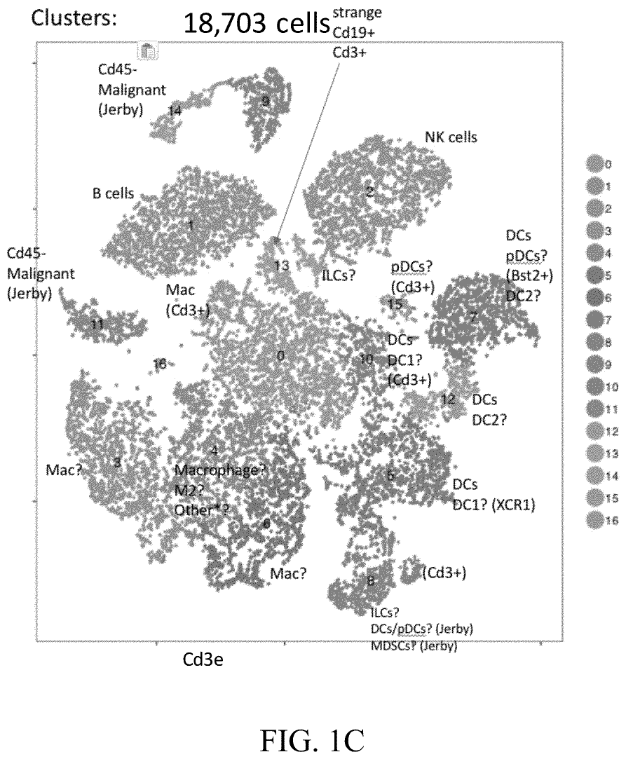

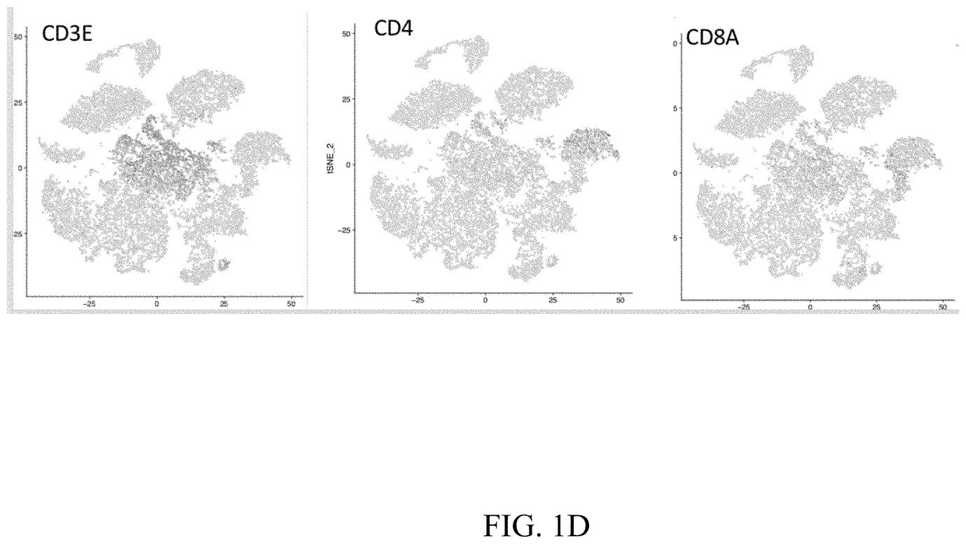

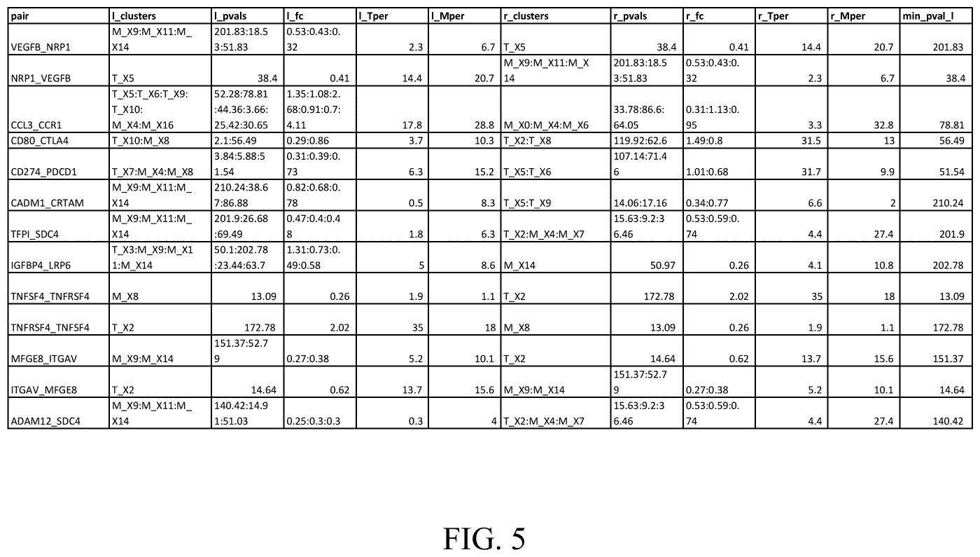

The CRISPR-Cas therapeutic methods disclosed herein may be designed for use with Class 1 CRISPR-Cas systems. In certain example embodiments, the Class 1 system may be Type I, Type III or Type IV CRISPR-Cas as described in Makarova et al. “Evolutionary classification of CRISPR-Cas systems: a burst of class 2 and derived variants” Nature Reviews Microbiology, 18:67-81 (February 2020), incorporated in its entirety herein by reference, and particularly as described in FIG. 1, p. 326. The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g. Cas1, Cas2, RNA nuclease), and/or one or more accessory proteins (e.g. Cas 4, DNA nuclease), CRISPR associated Rossman fold (CARF) domain containing proteins, and/or RNA transcriptase. Although Class 1 systems have limited sequence similarity, Class 1 system proteins can be identified by their similar architectures, including one or more Repeat Associated Mysterious Protein (RAMP) family subunits, e.g. Cas 5, Cas6, Cas7. RAMP proteins are characterized by having one or more RNA recognition motif domains. Large subunits (for example cas8 or cas10) and small subunits (for example, cas11) are also typical of Class 1 systems. See, e.g., FIGS. 1 and 2. Koonin E V, Makarova K S. 2019 Origins and evolution of CRISPR-Cas systems. Phil. Trans. R. Soc. B 374: 20180087, DOI: 10.1098/rstb.2018.0087. In one aspect, Class 1 systems are characterized by the signature protein Cas3. The Cascade in particular Class 1 proteins can comprise a dedicated complex of multiple Cas proteins that binds pre-crRNA and recruits an additional Cas protein, for example Cas6 or Cas5, which is the nuclease directly responsible for processing pre-crRNA. In one aspect, the Type I CRISPR protein comprises an effector complex comprises one or more Cas5 subunits and two or more Cas7 subunits. Class 1 subtypes include Type I-A, I-B, I-C, I-U, I-D, I-E, and I-F, Type IV-A and IV-B, and Type III-A, III-C, and III-B. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al, the CRISPR Journal, v. 1, n5, FIG. 5.

7(a)(4)—Class 2 Systems

The CRISPR-Cas therapeutic methods disclosed herein may be designed for use with. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside a split Ruv-C like nuclease domain sequence. The Type V systems (e.g., Cas12) only contain a RuvC-like nuclease domain that cleaves both strands. Some Type V systems have also been found to possess this collateral activity with two single-stranded DNA in in vitro contexts.

In one example embodiment, the Class 2 system is a Type II system. In one example embodiment, the Type II CRISPR-Cas system is a II-A CRISPR-Cas system. In one example embodiment, the Type II CRISPR-Cas system is a II-B CRISPR-Cas system. In one example embodiment, the Type II CRISPR-Cas system is a II-C1 CRISPR-Cas system. In one example embodiment, the Type II CRISPR-Cas system is a II-C2 CRISPR-Cas system. In some example embodiments, the Type II system is a Cas9 system. In some embodiments, the Type II system includes a Cas9.

In one example embodiment, the Class 2 system is a Type V system. In one example embodiment, the Type V CRISPR-Cas system is a V-A CRISPR-Cas system. In one example embodiment, the Type V CRISPR-Cas system is a V-B1 CRISPR-Cas system. In one example embodiment, the Type V CRISPR-Cas system is a V-B2 CRISPR-Cas system. In one example embodiment, the Type V CRISPR-Cas system is a V-C CRISPR-Cas system. In one example embodiment, the Type V CRISPR-Cas system is a V-D CRISPR-Cas system. In one example embodiment, the Type V CRISPR-Cas system is a V-E CRISPR-Cas system. In one example embodiment, the Type V CRISPR-Cas system is a V-F1 CRISPR-Cas system. In one example embodiment, the Type V CRISPR-Cas system is a V-F1 (V-U3) CRISPR-Cas system. In one example embodiment, the Type V CRISPR-Cas system is a V-F2 CRISPR-Cas system. In one example embodiment, the Type V CRISPR-Cas system is a V-F3 CRISPR-Cas system. In one example embodiment, the Type V CRISPR-Cas system is a V-G CRISPR-Cas system. In one example embodiment, the Type V CRISPR-Cas system is a V-H CRISPR-Cas system. In one example embodiment, the Type V CRISPR-Cas system is a V-I CRISPR-Cas system. In one example embodiment, the Type V CRISPR-Cas system is a V-K (V-U5) CRISPR-Cas system. In one example embodiment, the Type V CRISPR-Cas system is a V-U1 CRISPR-Cas system. In one example embodiment, the Type V CRISPR-Cas system is a V-U2 CRISPR-Cas system. In one example embodiment, the Type V CRISPR-Cas system is a V-U4 CRISPR-Cas system. In one example embodiment, the Type V CRISPR-Cas is a Cas12a (Cpf1), Cas12b (C2c1), Cas12c (C2c3), Cas12d (CasY), Cas12e (CasX), Cas14, and/or CasΦ.

7(a)(5)—Guide Molecules

The following include general design principles that may be applied to the guide molecule. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In one example embodiment, a guide RNA or crRNA may comprise, consist essentially of, or consist of a direct repeat (DR) sequence and a guide sequence or spacer sequence. In another example embodiment, the guide RNA or crRNA may comprise, consist essentially of, or consist of a direct repeat sequence fused or linked to a guide sequence or spacer sequence. In another example embodiment, the direct repeat sequence may be located upstream (i.e., 5′) from the guide sequence or spacer sequence. In other embodiments, the direct repeat sequence may be located downstream (i.e., 3′) from the guide sequence or spacer sequence.

In one example embodiment, the crRNA comprises a stem loop, preferably a single stem loop. In one example embodiment, the direct repeat sequence forms a stem loop, preferably a single stem loop.

In one example embodiment, the spacer length of the guide RNA is from 15 to 35 nt. In another example embodiment, the spacer length of the guide RNA is at least 15 nucleotides. In another example embodiment, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All of (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

7(a)(6)—Target Sequences, PAMs, and PFSs

In the context of formation of a CRISPR complex, “target sequence” refers to a sequence to which a guide sequence is designed to have complementarity, where hybridization between a target sequence and a guide sequence promotes the formation of a CRISPR complex. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

PAM elements are sequences that can be recognized and bound by Cas proteins. Cas proteins/effector complexes can then unwind the dsDNA at a position adjacent to the PAM element. It will be appreciated that Cas proteins and systems target RNA do not require PAM sequences (Marraffini et al. 2010. Nature. 463:568-571). Instead, many rely on PFSs, which are discussed elsewhere herein. In one example embodiment, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table A (from Gleditzsch et al. 2019) below shows several Cas polypeptides and the PAM sequence they recognize.

TABLE A
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including | TTTV |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |
| Cas1 | 5′-CTT-3′ |
| Cas8e | 5′-ATG-3′ |
| Type I-A | 5′-CCN-3′ |
| Type I-B | TTC, ACT, TAA, TAT, TAG, and CAC |
| Type I-C | NTTC |
| Type I-E | 5′-AAG-3′ |
| TypeI-F | GG |

In a preferred embodiment, the CRISPR effector protein may recognize a 3′ PAM. In one example embodiment, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016.Mol. Cell. 16:253), and negative screening (Zetsche et al. 2015. Cell. 163:759-771).

As previously mentioned, CRISPR-Cas systems that target RNA do not typically rely on PAM sequences. Instead such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′ end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

7(a)(7)—Sequences Related to Nucleus Targeting and Transportation

In some embodiments, one or more components (e.g., the Cas protein) in the composition for engineering cells may comprise one or more sequences related to nucleus targeting and transportation. Such sequences may facilitate the one or more components in the composition for targeting a sequence within a cell. In order to improve targeting of the CRISPR-Cas protein used in the methods of the present disclosure to the nucleus, it may be advantageous to provide one or both of these components with one or more nuclear localization sequences (NLSs).

In one example embodiment, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO:6) or PKKKRKVEAS (SEQ ID NO:7); the NLS from nucleoplasmin (e.g., the nucleoplasmin bipartite NLS with the sequence KRPAATKKAGQAKKKK (SEQ ID NO:8)); the c-myc NLS having the amino acid sequence PAAKRVKLD (SEQ ID NO:9) or RQRRNELKRSP (SEQ ID NO:10); the hRNPA1 M9 NLS having the sequence NQSSNFGPMKGGNFGGRSSGPYGGGGQYFAKPRNQGGY (SEQ ID NO:11); the sequence RMRIZFKNKGKDTAELRRRRVEVSVELRKAKKDEQILKRRNV (SEQ ID NO:12) of the IBB domain from importin-alpha; the sequences VSRKRPRP (SEQ ID NO:13) and PPKKARED (SEQ ID NO:14) of the myoma T protein; the sequence PQPKKKPL (SEQ ID NO:15) of human p53; the sequence SALIKKKKKMAP (SEQ ID NO:16) of mouse c-abl IV; the sequences DRLRR (SEQ ID NO:17) and PKQKKRK (SEQ ID NO:18) of the influenza virus NS1; the sequence RKLKKKIKKL (SEQ ID NO:19) of the Hepatitis virus delta antigen; the sequence REKKKFLKRR (SEQ ID NO:20) of the mouse Mx1 protein; the sequence KRKGDEVDGVDEVAKKKSKK (SEQ ID NO:21) of the human poly(ADP-ribose) polymerase; and the sequence RKCLQAGMNLEARKTKK (SEQ ID NO:22) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the Cas protein, or exposed to a Cas protein lacking the one or more NLSs.

The Cas proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the Cas proteins, an NLS attached to the C-terminal of the protein.

7b—Base Editing

Provided herein are methods for editing DNA or RNA, i.e., base editing, without inducing double-stranded breaks in the DNA targeted for modification. In one example embodiment, the PENK sequence is edited to alter one or more protease cleavage sites to prevent production of MENK and PENK peptides. A base-editing system may comprise a Cas polypeptide linked to a nucleobase deaminase (“base editing system”) and a guide molecule capable of forming a complex with the Cas polypeptide and directing sequence-specific binding of the base editing system at a target sequence. In one example embodiment, the Cas polypeptide is catalytically inactive. In another example embodiment, the Cas polypeptide is a nickase. The Cas polypeptide may be any of the Cas polypeptides disclosed above. In one example embodiment, the Cas polypeptide is a Type II Cas polypeptide. In one example embodiment, the Cas polypeptide is a Cas9 polypeptide. In another example embodiment, the Cas polypeptide is a Type V Cas polypeptide. In one example embodiment, the Cas polypeptide is a Cas12a or Cas12b polypeptide. The nucleobase deaminase may be cytosine base editor (CBE) or adenosine base editors (ABEs). CBEs convert C•G base pairs into a T•A base pair (Komor et al. 2016. Nature. 533:420-424; Nishida et al. 2016. Science. 353; and Li et al. Nat. Biotech. 36:324-327) and ABEs convert an A•T base pair to a G•C base pair. Collectively, CBEs and ABEs can mediate all four possible transition mutations (C to T, A to G, T to C, and G to A). Example base editing systems are disclosed in Rees and Liu (2018), Nat. Rev. Genet. 19(12): 770-788, particularly at FIGS. 1b, 2a-2c, 3a-3f, and Table 1, which is specifically incorporated herein by reference. In certain example embodiments, the base editing system may further comprise a DNA glycosylase inhibitor.

In one example embodiment, a method of enhancing the anti-tumor immunity of a subject in need thereof comprises administering a base editing system that generates one or more variants with decreased expression or activity of PENK.

The editing window of a base editing system may range over a 5-8 nucleotide window, depending on the base editing system used. Id. Accordingly, given the base editing system used, a guide sequence may be selected to direct the base editing system to convert a base or base pair of one or more variants resulting in reduced PENK expression. In one example embodiment, the base editing system, may be used to introduce a pre-mature stop codon in the genomic DNA sequence encoding PENK. In another example embodiment, the base editing system may be used to the cleavage recognition site of PENK to prevent generation of PENK-derived signaling peptides. In another example embodiment, the based editing system may be used to modify a post-translation modification site on an mRNA expressed from a PENK gene, either through introduction or removal of a post-translational modification site, that results in reduced PENK function or reduced function in PENK derived polypeptides.

7(b)(1)—DNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In some embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C•G base pair into a T•A base pair (Komor et al. 2016. Nature. 533:420-424; Nishida et al. 2016. Science. 353; and Li et al. Nat. Biotech. 36:324-327) and ABEs convert an A•T base pair to a G•C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471. Base editors may be further engineered to optimize conversion of nucleotides (e.g. A:T to G:C). Richter et al. 2020. Nature Biotechnology. doi.org/10.1038/s41587-020-0453-z.

Other Example Type V base editing systems are described in International Patent Publication Nos. WO 2018/213708, WO 2018/213726, and International Patent Applications No. PCT/US2018/067207, PCT/US2018/067225, and PCT/US2018/067307, each of which is incorporated herein by reference.

7(b)(2)—RNA Base Editing

The present application relates, in part, to modifying a target RNA sequence of interest. In certain example embodiments, the base editing system may be a RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA based editor may be used to delete or introduce a post-translational modification site in the expressed mRNA. In contrast to DNA base editors, whose edits are permanent in the modified cell, RNA base editors can provide edits where finer temporal control may be needed, for example in modulating a particular immune response. Example Type VI RNA-base editing systems are described in Cox et al. 2017. Science 358: 1019-1027, WO 2019/005884, WO 2019/005886, WO 2019/071048, PCT/US20018/05179, PCT/US2018/067207, which are incorporated herein by reference. An example FnCas9 system that may be adapted for RNA base editing purposes is described in WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

Using RNA-targeting rather than DNA targeting offers several advantages relevant for therapeutic development. First, there are substantial safety benefits to targeting RNA: there will be fewer off-target events because the available sequence space in the transcriptome is significantly smaller than the genome, and if an off-target event does occur, it will be transient and less likely to induce negative side effects. Second, RNA-targeting therapeutics will be more efficient because they are cell-type independent and do not have to enter the nucleus, making them easier to deliver.

In an embodiment, the present disclosure includes an engineered composition for site-directed base editing comprising: a targeting domain; and an adenosine deaminase or catalytic domain thereof, wherein the adenosine deaminase is modified to convert activity to a cytidine deaminase.

In some embodiments, the adenosine deaminase is modified by one or more mutations at one or more positions selected from E396, C451, V351, R455, T375, K376, 5486, Q488, R510, K594, R348, G593, 5397, H443, L444, Y445, F442, E438, T448, A353, V355, T339, P539, V525 and I520. In some embodiments, the adenosine deaminase is mutated at one or more positions selected from E488, V351, S486, T375, 5370, P462, and N597. In some embodiments, the adenosine deaminase comprises one or more mutations selected from E488Q, V351G, S486A, T375S, S370C, P462A, and N597I. In some embodiments, the adenosine deaminase protein or catalytic domain thereof is a human, cephalopod, or Drosophila adenosine deaminase protein or catalytic domain thereof. In some embodiments, said adenosine deaminase protein or catalytic domain thereof has been modified to comprise a mutation at glutamic acid488 of the hADAR2-D amino acid sequence, or a corresponding position in a homologous ADAR protein. In some embodiments, said glutamic acid residue at position 488 or a corresponding position in a homologous ADAR protein is replaced by a glutamine residue (E488Q). In some embodiments, said adenosine deaminase protein or catalytic domain thereof is a mutated hADAR2d comprising mutation E488Q or a mutated hADAR1d comprising mutation E1008Q. In some embodiments, the targeting domain is a catalytically inactive Cas13 protein, or a nucleotide sequence encoding said catalytically inactive Cas13 protein. In some embodiments, the catalytically inactive Cas13 protein is catalytically inactive Cas13a, catalytically inactive Cas13b, or catalytically inactive Cas13c. In some embodiments, said catalytically inactive Cas13 protein is obtained from a Cas13 nuclease derived from a bacterial species selected from the group consisting of the bacterial species listed in any of Tables 1, 2, 3, or 4. In some embodiments, the composition further comprising a guide molecule which comprises a guide sequence linked to a direct repeat sequence, or a nucleotide sequence encoding said guide molecule. In some embodiments, said adenosine deaminase protein or catalytic domain thereof is covalently or non-covalently linked to the targeting domain.

In another embodiment, the disclosure relates to a method of modifying an Adenine in a target RNA sequence of interest. In particular embodiments, the method comprises delivering to said target RNA: (a) a catalytically inactive (dead) Cas13 protein; (b) a guide molecule which comprises a guide sequence linked to a direct repeat sequence; and (c) an adenosine deaminase protein or catalytic domain thereof; wherein said adenosine deaminase protein or catalytic domain thereof is covalently or non-covalently linked to said dead Cas13 protein or said guide molecule or is adapted to link thereto after delivery; wherein guide molecule forms a complex with said dead Cas13 protein and directs said complex to bind said target RNA sequence of interest, wherein said guide sequence is capable of hybridizing with a target sequence comprising said Adenine to form an RNA duplex, wherein said guide sequence comprises a non-pairing Cytosine at a position corresponding to said Adenine resulting in an A-C mismatch in the RNA duplex formed; wherein said adenosine deaminase protein or catalytic domain thereof deaminates said Adenine in said RNA duplex.

In some embodiments, the RNA editing is carried out using the Cas13 protein, wherein the Cas13 protein is Cas13a, Cas13b or Cas13c.

The adenosine deaminase protein or catalytic domain thereof is fused to N- or C-terminus of said dead Cas13 protein. In some embodiments, the adenosine deaminase protein or catalytic domain thereof is fused to said dead Cas13 protein by a linker.

In some embodiments, the adenosine deaminase protein or catalytic domain thereof is linked to an adaptor protein and said guide molecule or said dead Cas13 protein comprises an aptamer sequence capable of binding to said adaptor protein. The adaptor sequence may be selected from MS2, PP7, Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s and PRR1.

In some embodiments, the adenosine deaminase protein or catalytic domain thereof is inserted into an internal loop of said dead Cas13 protein. In some embodiments, the Cas13a protein comprises one or more mutations in the two HEPN domains, particularly at position R474 and R1046 of Cas 13a protein originating from Leptotrichia wadei or amino acid positions corresponding thereto of a Cas13a ortholog.

In some embodiments, the Cas13 protein is a Cas13b proteins, and the Cas13b comprises a mutation in one or more of positions R116, H121, R1177, H1182 of Cas13b protein originating from Bergeyella zoohelcum ATCC 43767 or amino acid positions corresponding thereto of a Cas13b ortholog. In some embodiments, the mutation is one or more of R116A, H121A, R1177A, H1182A of Cas13b protein originating from Bergeyella zoohelcum ATCC 43767 or amino acid positions corresponding thereto of a Cas13b ortholog.

In some embodiments, the guide sequence has a length of about 29-53 nt capable of forming said RNA duplex with said target sequence. In some embodiments, the guide sequence has a length of about 40-50 nt capable of forming said RNA duplex with said target sequence. In some embodiments, the distance between said non-pairing C and the 5′ end of said guide sequence is 20-30 nucleotides.

In some embodiments, the adenosine deaminase protein or catalytic domain thereof is a human, cephalopod, or Drosophila adenosine deaminase protein or catalytic domain thereof. In certain example embodiments, the adenosine deaminase protein or catalytic domain thereof has been modified to comprise a mutation at glutamic acid⁴⁸⁸ of the hADAR2-D amino acid sequence, or a corresponding position in a homologous ADAR protein. In some embodiments, the glutamic acid residue may be at position 488 or a corresponding position in a homologous ADAR protein is replaced by a glutamine residue (E488Q).

In some embodiments, the adenosine deaminase protein or catalytic domain thereof is a mutated hADAR2d comprising mutation E488Q or a mutated hADAR1d comprising mutation E1008Q.

In some embodiments, the guide sequence comprises more than one mismatch corresponding to different adenosine sites in the target RNA sequence or wherein two guide molecules are used, each comprising a mismatch corresponding to a different adenosine sites in the target RNA sequence.

In some embodiments, the Cas13 protein and optionally said adenosine deaminase protein or catalytic domain thereof comprise one or more heterologous nuclear localization signal(s) (NLS(s)).

In some embodiments, the method further comprises, determining the target sequence of interest and selecting an adenosine deaminase protein or catalytic domain thereof which most efficiently deaminates said adenine present in then target sequence.

The components of the systems described herein may be delivered to said cell as a ribonucleoprotein complex or as one or more polynucleotide molecules. The one or more polynucleotide molecules may comprise one or more mRNA molecules encoding the components. The one or more polynucleotide molecules may be comprised within one or more vectors. The one or more polynucleotide molecules may further comprise one or more regulatory elements operably configured to express said Cas13 protein, said guide molecule, and said adenosine deaminase protein or catalytic domain thereof, optionally wherein said one or more regulatory elements comprise inducible promoters. The one or more polynucleotide molecules or said ribonucleoprotein complex may be delivered via particles, vesicles, or one or more viral vectors. The particles may comprise a lipid, a sugar, a metal or a protein. The particles may comprise lipid nanoparticles. The vesicles may comprise exosomes or liposomes. The one or more viral vectors may comprise one or more of adenovirus, one or more lentivirus or one or more adeno-associated virus.

The RNA editing methods disclosed herein may be used to modify a cell, a cell line or an organism by manipulation of one or more target RNA sequences.

In some embodiments, the deamination of said Adenine in said target RNA of interest remedies a disease caused by transcripts containing a pathogenic G→A or C→T point mutation.

The methods disclosed herein, may be used to make a modification that affects specific, targeted genes of an organism (e.g., PENK). The modification may affect splicing of said target RNA sequence. The modification may introduce a mutation in a transcript that reduces expression of the targeted gene. The modification may introduce an amino acid change and cause a reduction in activity of the targeted protein.

In some embodiments, the deamination of the adenine in said target RNA of interest causes a loss of function or reduced expression of a gene. In certain example embodiments, the loss of function or reduced expression of the gene leads to an enhancement of anti-tumor immunity in a subject.

In some embodiments, the cytosine of the adenosine deaminase is not 5′ flanked by guanosine. In certain embodiments, said adenosine deaminase is ADAR, optionally huADAR, optionally (hu)ADAR1 or (hu)ADAR2. In certain embodiments, said Cas13, preferably Cas13b, is truncated, preferably C-terminally truncated, preferably wherein said Cas 13 is a truncated functional variant of the corresponding wild type Cas13.

In another aspect, the present disclosure includes a method of modifying an Adenine in a target RNA sequence of interest, comprising delivering to said target RNA: (a) a catalytically inactive (dead) Cas13 protein; (b) a guide molecule which comprises a guide sequence linked to a direct repeat sequence; and (c) an adenosine deaminase protein or catalytic domain thereof mutated to convert activity to a cytidine deaminase; wherein said adenosine deaminase protein or catalytic domain thereof is covalently or non-covalently linked to said dead Cas13 protein or said guide molecule or is adapted to link thereto after delivery; wherein said guide molecule forms a complex with said dead Cas13 protein and directs said complex to bind said target RNA sequence of interest, wherein said guide sequence is capable of hybridizing with a target sequence comprising said adenine to form an RNA duplex, wherein said guide sequence comprises a non-pairing cytosine at a position corresponding to said adenine resulting in an A-C mismatch in the RNA duplex formed; wherein said adenosine deaminase protein or catalytic domain thereof deaminates said adenine in said RNA duplex.

In some embodiments, the adenosine deaminase is mutated at one or more positions selected from E396, C451, V351, R455, T375, K376, S486, Q488, R510, K594, R348, G593, S397, H443, L444, Y445, F442, E438, T448, A353, V355, T339, P539, V525 and I520. In some example embodiments, the adenosine deaminase is mutated at one or more positions selected from E488, V351, S486, T375, S370, P462, and N597.

In some embodiments, the present disclosure includes an engineered, non-naturally occurring RNA editing system suitable for modifying an adenine in a target locus of interest, comprising (a) a guide molecule which comprises a guide sequence linked to a direct repeat sequence, or a nucleotide sequence encoding said guide molecule; (b) a catalytically inactive Cas13 protein, or a nucleotide sequence encoding said catalytically inactive Cas13 protein; (c) an adenosine deaminase protein or catalytic domain thereof, or a nucleotide sequence encoding said adenosine deaminase protein or catalytic domain thereof, wherein the adenosine deaminase is modified to convert activity to a cytidine deaminase; wherein said adenosine deaminase protein or catalytic domain thereof is covalently or non-covalently linked to said Cas13 protein or said guide molecule or is adapted to link thereto after delivery; wherein said guide sequence is capable of hybridizing with a target RNA sequence comprising an adenine to form an RNA duplex, wherein said guide sequence comprises a non-pairing cytosine at a position corresponding to said Adenine resulting, in an A-C mismatch in the RNA duplex formed.

In some embodiments, the adenosine deaminase is modified by one or more mutations selected from E396, C451, V351, R455, T375, K376, S486, Q488, R510, K594, R348, G593, S397, H443, L444, Y445, F442, E438, T448, A353, V355, T339, P539, V525 and I520. In some embodiments, the adenosine deaminase is mutated at one or more positions selected from E488, V351, S486, T375, S370, P462, and N597.

### 7c.—Prime Editing

In one example embodiment, a method of enhancing the anti-tumor immunity of a subject in need thereof comprises administering a prime editing system that generates one or more variants with decreased expression or activity of PENK. Like base editing systems, prime editing systems are capable of targeted modification of a polynucleotides without generating double stranded breaks. See e.g. Anzalone et al. 2019. Nature. 576: 149-157, incorporated herein by reference. Prime editing can operate via a “search-and-replace” methodology and can mediate targeted insertions, deletions, as well as all 12 possible base-to-base conversion and combinations thereof. In one example embodiment, prime editing may introduce a pre-mature stop codon into the genomic DNA sequence encoding PENK. In another example embodiment, prime editing may be used to insert a fragment into the genomic DNA sequence encoding PENK that renders the gene or gene product non-functional.

In one example embodiment, a prime editing system comprises a Cas polypeptide having nickase activity, a reverse transcriptase, and a prime editing guide RNA (pegRNA). Cas polypeptide, and/or reverse transcriptase can be coupled together or otherwise associate with each other to form a prime editing complex and edit a target sequence. The Cas polypeptide may be any of the Cas polypeptides disclosed above. In one example embodiment, the Cas polypeptide is a Type II Cas polypeptide. In another example embodiment, the Cas polypeptide is a Cas9 nickase. In one example embodiment, the Cas polypeptide is a Type V Cas polypeptide. In another example embodiment, the Cas polypeptide is a Cas12a or Cas12b.

The prime editing guide molecule (pegRNA) comprises a primer binding site (PBS) configured to hybridize with a portion of a nicked strand on a target polynucleotide (e.g. genomic DNA) a reverse transcriptase (RT) template comprising the edit to be inserted in the genomic DNA and a spacer sequence designed to hybridize to a target sequence at the site of the desired edit. The nicking site is dependent on the Cas polypeptide used and standard cutting preference for that Cas polypeptide relative to the PAM. Thus, based on the Cas polypeptide used, a pegRNA can be designed to direct the prime editing system to introduce a nick where the desired edit should take place. In one example embodiment, a pegRNA is configured to direct the prime editing system to convert a single base or base pair of the one or more variants associated with reduced PENK expression. In one example embodiment, a pegRNA is configured to direct the prime editing system to convert a single base or base pair of one or more variants associated with reduced PENK expression such that PENK protein activity is reduced.

The pegRNA can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c

### 7d.—CAST

In one example embodiment, a method of enhancing the anti-tumor immunity of a subject in need thereof comprises administering a CAST system that incorporates or inserts a genomic region comprising one or more variants associated with decreased expression or activity of PENK. In one example embodiment, a CAST system is used to replace all or a portion of PENK comprising one or more variants that reduce PENK expression or activity. In one example embodiment, the variant is generated using one or more of the gene editing methods described herein.

CAST systems comprise a Cas polypeptide, a guide sequence, a transposase, and a donor construct. The transposase is linked to or otherwise capable of forming a complex with the Cas polypeptide. The donor construct comprises a donor sequence to be inserted into a target polynucleotide and one or more transposase recognition elements. The transposase is capable of binding the donor construct and excising the donor template and directing insertion of the donor template into a target site on a target polynucleotide (e.g. genomic DNA). The guide molecule is capable of forming a CRISPR-Cas complex with the Cas polypeptide, and can be programmed to direct the entire CAST complex such that the transposase is positioned to insert the donor sequence at the target site on the target polynucleotide. For multimeric transposase, only those transpoases needed for recognition of the donor construct and transposition of the donor sequence into the target polypeptide may be required. The Cas may be naturally catalytically inactive or engineered to be catalyically inactive.

In one example embodiment, the CAST system is a Tn7-like CAST system, wherein the transposase comprises one or more polypeptides from a Tn7 or Tn7-like transposase. The Cas polypeptide of the Tn7-like transposase may be a Class 1 (multimeric effector complex) or Class 2 (single protein effector) Cas polypeptide.

In one example embodiments, the Cas polypeptide is a Class 1 Type-1f Cas polypeptide. In one example embodiment, the Cas polypeptide may comprise a cash, a cas7, and a cas8-cas5 fusion. In one example embodiments, the Tn7 transposase may comprise TnsB, TnsC, and TniQ. In another example embodiment, the Tn7 transposase may comprise TnsB, TnsC, and TnsD. In certain example embodiments, the Tn7 transposase may comprise TnsD, TnsE, or both. As used herein, the terms “TnsAB”, “TnsAC”, “TnsBC”, or “TnsABC” refer to a transponson complex comprising TnsA and TnsB, TnsA and TnsC, TnsB and TnsC, TnsA and TnsB and TnsC, respectively. In these combinations, the transposases (TnsA, TnsB, TnsC) may form complexes or fusion proteins with each other. Similarly, the term TnsABC-TniQ refer to a transposon comprising TnsA, TnsB, TnsC, and TniQ, in a form of complex or fusion protein. An example Type 1f-Tn7 CAST system is described in Klompe et al. Nature, 2019, 571:219-224 and Vo et al. bioRxiv, 2021, doi.org/10.1101/2021.02.11.430876, which are incorporated herein by reference.

In one example embodiment, the Cas polypeptide is a Class 1 Type-1b Cas polypeptide. In one example embodiment, the Cas polypeptide may comprise a cash, a cas7, and a cas8b (e.g. a ca8b3). In one example embodiments, the Tn7 transposase may comprise TnsB, TnsC, and TniQ. In another example embodiment, the Tn7 transposase may comprise TnsB, TnsC, and TnsD. In certain example embodiments, the Tn7 transposase may comprise TnsD, TnsE, or both. As used herein, the terms “TnsAB”, “TnsAC”, “TnsBC”, or “TnsABC” refer to a transponson complex comprising TnsA and TnsB, TnsA and TnsC, TnsB and TnsC, TnsA and TnsB and TnsC, respectively. In these combinations, the transposases (TnsA, TnsB, TnsC) may form complexes or fusion proteins with each other. Similarly, the term TnsABC-TniQ refer to a transposon comprising TnsA, TnsB, TnsC, and TniQ, in a form of complex or fusion protein.

In one example embodiment, the Cas polypeptide is Class 2, Type V Cas polypeptide. In one example embodiment, the Type V Cas polypeptide is a Cas12k. In one example embodiments, the Tn7 transposase may comprise TnsB, TnsC, and TniQ. In another example embodiment, the Tn7 transposase may comprise TnsB, TnsC, and TnsD. In certain example embodiments, the Tn7 transposase may comprise TnsD, TnsE, or both. As used herein, the terms “TnsAB”, “TnsAC”, “TnsBC”, or “TnsABC” refer to a transponson complex comprising TnsA and TnsB, TnsA and TnsC, TnsB and TnsC, TnsA and TnsB and TnsC, respectively. In these combinations, the transposases (TnsA, TnsB, TnsC) may form complexes or fusion proteins with each other. Similarly, the term TnsABC-TniQ refer to a transposon comprising TnsA, TnsB, TnsC, and TniQ, in a form of complex or fusion protein. An example Cas12k-Tn7 CAST system is described in Strecker et al. Science, 2019 365:48-53, which is incorporated herein by reference.

In one example embodiment, the CAST system is a Mu CAST system, wherein the transposase comprises one or more polypeptides of a Mu transposase. An example Mu CAST system is disclosed in WO/2021/041922 which is incorporated herein by reference.

In one example embodiment, the CAST comprise a catalytically inactive Type II Cas polypeptide (e.g. dCas9) fused to one or more polypeptides of a Tn5 transposase. In another example embodiment, the CAST system comprises a catalytically inactive Type II Cas polypeptide (e.g. dCas9) fused to a piggyback transposase

The system may further comprise one or more donor polynucleotides (e.g., for insertion into the target polynucleotide). A donor polynucleotide may be an equivalent of a transposable element that can be inserted or integrated to a target site. The donor polynucleotide may be or comprise one or more components of a transposon. A donor polynucleotide may be any type of polynucleotides, including, but not limited to, a gene, a gene fragment, a non-coding polynucleotide, a regulatory polynucleotide, a synthetic polynucleotide, etc. The donor polynucleotide may include a transposon left end (LE) and transposon right end (RE). The LE and RE sequences may be endogenous sequences for the CAST used or may be heterologous sequences recognizable by the CAST used, or the LE or RE may be synthetic sequences that comprise a sequence or structure feature recognized by the CAST and sufficient to allow insertion of the donor polynucleotide into the target polynucleotides. In certain example embodiments, the LE and RE sequences are truncated. In certain example embodiments may be between 100-200 bps, between 100-190 base pairs, 100-180 base pairs, 100-170 base pairs, 100-160 base pairs, 100-150 base pairs, 100-140 base pairs, 100-130 base pairs, 100-120 base pairs, 100-110 base pairs, 20-100 base pairs, 20-90 base pairs, 20-80 base pairs, 20-70 base pairs, 20-60 base pairs, 20-50 base pairs, 20-40 base pairs, 20-30 base pairs, 50 to 100 base pairs, 60-100 base pairs, 70-100 base pairs, 80-100 base pairs, or 90-100 base pairs in length

The donor polynucleotide may be inserted at a position upstream or downstream of a PAM on a target polynucleotide. In some embodiments, a donor polynucleotide comprises a PAM sequence. Examples of PAM sequences include TTTN, ATTN, NGTN, RGTR, VGTD, or VGTR.

The donor polynucleotide may be inserted at a position between 10 bases and 200 bases, e.g., between 20 bases and 150 bases, between 30 bases and 100 bases, between 45 bases and 70 bases, between 45 bases and 60 bases, between 55 bases and 70 bases, between 49 bases and 56 bases or between 60 bases and 66 bases, from a PAM sequence on the target polynucleotide. In some cases, the insertion is at a position upstream of the PAM sequence. In some cases, the insertion is at a position downstream of the PAM sequence. In some cases, the insertion is at a position from 49 to 56 bases or base pairs downstream from a PAM sequence. In some cases, the insertion is at a position from 60 to 66 bases or base pairs downstream from a PAM sequence.

The donor polynucleotide may be used for editing the target polynucleotide. In some cases, the donor polynucleotide comprises one or more mutations to be introduced into the target polynucleotide. Examples of such mutations include substitutions, deletions, insertions, or a combination thereof. The mutations may cause a shift in an open reading frame on the target polynucleotide. In some cases, the donor polynucleotide alters a stop codon in the target polynucleotide. For example, the donor polynucleotide may correct a premature stop codon. The correction may be achieved by deleting the stop codon or introduces one or more mutations to the stop codon. In other example embodiments, the donor polynucleotide addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment refers to less than the entire copy of a gene by providing sufficient nucleotide sequence to restore the functionality of a wild type gene or non-coding regulatory sequence (e.g. sequences encoding long non-coding RNA). In certain example embodiments, the systems disclosed herein may be used to replace a single allele of a defective gene or defective fragment thereof. In another example embodiment, the systems disclosed herein may be used to replace both alleles of a defective gene or defective gene fragment. A “defective gene” or “defective gene fragment” is a gene or portion of a gene that when expressed fails to generate a functioning protein or non-coding RNA with functionality of a corresponding wild-type gene. In certain example embodiments, these defective genes may be associated with one or more disease phenotypes. In certain example embodiments, the defective gene or gene fragment is not replaced but the systems described herein are used to insert donor polynucleotides that encode gene or gene fragments that compensate for or override defective gene expression such that cell phenotypes associated with defective gene expression are eliminated or changed to a different or desired cellular phenotype.

In certain embodiments of the invention, the donor may include, but not be limited to, genes or gene fragments, encoding proteins or RNA transcripts to be expressed, regulatory elements, repair templates, and the like. According to the invention, the donor polynucleotides may comprise left end and right end sequence elements that function with transposition components that mediate insertion.

In certain cases, the donor polynucleotide manipulates a splicing site on the target polynucleotide. In some examples, the donor polynucleotide disrupts a splicing site. The disruption may be achieved by inserting the polynucleotide to a splicing site and/or introducing one or more mutations to the splicing site. In certain examples, the donor polynucleotide may restore a splicing site. For example, the polynucleotide may comprise a splicing site sequence.

The donor polynucleotide to be inserted may have a size from 10 bases to 50 kb in length, e.g., from 50 to 40 kb, from 100 to 30 kb, from 100 bases to 300 bases, from 200 bases to 400 bases, from 300 bases to 500 bases, from 400 bases to 600 bases, from 500 bases to 700 bases, from 600 bases to 800 bases, from 700 bases to 900 bases, from 800 bases to 1000 bases, from 900 bases to from 1100 bases, from 1000 bases to 1200 bases, from 1100 bases to 1300 bases, from 1200 bases to 1400 bases, from 1300 bases to 1500 bases, from 1400 bases to 1600 bases, from 1500 bases to 1700 bases, from 600 bases to 1800 bases, from 1700 bases to 1900 bases, from 1800 bases to 2000 bases, from 1900 bases to 2100 bases, from 2000 bases to 2200 bases, from 2100 bases to 2300 bases, from 2200 bases to 2400 bases, from 2300 bases to 2500 bases, from 2400 bases to 2600 bases, from 2500 bases to 2700 bases, from 2600 bases to 2800 bases, from 2700 bases to 2900 bases, or from 2800 bases to 3000 bases in length.

The components in the systems herein may comprise one or more mutations that alter their (e.g., the transposase(s)) binding affinity to the donor polynucleotide. In some examples, the mutations increase the binding affinity between the transposase(s) and the donor polynucleotide. In certain examples, the mutations decrease the binding affinity between the transposase(s) and the donor polynucleotide. The mutations may alter the activity of the Cas and/or transposase(s).

In certain embodiments, the systems disclosed herein are capable of unidirectional insertion, that is the system inserts the donor polynucleotide in only one orientation.

### 7e.—TALENs

As disclosed herein editing can be made by way of the transcription activator-like effector nucleases (TALENs) system. In some embodiments, the programmable nuclease may be a transcription activator-like effector nuclease (TALEN), a functional fragment thereof, or a variant thereof. The present disclosure also includes nucleotide sequences that are or encode one or more components of a TALEN system. As disclosed herein, editing can be made by way of the transcription activator-like effector (TALEs) system, which have been used to modify endogenous genes in various species, including viruses. yeast, plants, nematodes, insects, frogs. fish and mammals such as mice, rats and pigs, as well as in cultured mammalian cells.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference. The structure and function of TALEs is further described in, for example, Moscou et al., Science 326:1501 (2009); Boch et al., Science 326:1509-1512 (2009); and Zhang et al., Nature Biotechnology 29:149-153 (2011).

In some embodiments, provided herein include isolated, non-naturally occurring, recombinant or engineered DNA binding proteins that comprise TALE monomers as a part of their organizational structure that enable the targeting of nucleic acid sequences with improved efficiency and expanded specificity.

In one example embodiment, a method of enhancing the anti-tumor immunity of a subject in need thereof comprises administering a TALENs editing system that generates variants with decreased expression or activity of PENK.

In some embodiments, TALENs can be designed to target almost any given DNA sequence, which is a crucial advantage of TALENs over other types of nucleases. For example, small DNA sequences (such as enhancers or miRNA-coding sequences) may lack targetable sites for ZFNs or CRISPR-Cas systems but can be mutated preferentially using TAT ENs. The only limitation in the design of TALENs seems to be the requirement for a thymine at the 5′ end of the target sequence, which is recognized by two amino-terminal cryptic repeat folds. Although there have been conflicting reports that emphasize or refute the importance of this 5′ thymine, choosing a target sequence with a thymine at the 5′ end is usually recommended. Recently developed TALE variants that recognize other bases at the 5′ end would further broaden the range of targetable sites. Conventional TALENs cannot cleave target DNA that contains methylated cytosines. However, a methylated cytosine is indistinguishable from a thymine in the major groove; hence, the His-Asp RVD repeat (which recognizes cytosines) can be replaced with an Asn-Gly RVD repeat (which recognizes thymine) to generate TALENs that can cleave methylated DNA.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

### 7f.—Zn Finger Nucleases

Other preferred tools for genome editing for use in the context of this invention include zinc finger systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP). Zinc-finger nuclease (ZFNs) have been used to modify endogenous genes in various organisms, including viruses, bacteria, nematodes, frogs, plants, insects, fish and mammals such as mice, rats and pigs, as well as in cultured mammalian and avian cells.

In one example embodiment, a method of enhancing the anti-tumor immunity of a subject in need thereof comprises administering a zinc-finger nuclease editing system that generates variants with decreased expression or activity of PENK.

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### 7g.—Meganucleases

In some embodiments, the programmable nuclease may be a meganuclease or system thereof. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated by reference.

In one example embodiment, a method of enhancing the anti-tumor immunity of a subject in need thereof comprises administering a meganuclease or system thereof that generates variants with decreased expression or activity of PENK.

### PENK Example Modifications

PENK-specific mutations may be introduced using the gene editing systems described above. In one example embodiment, one or more cleavage sites are modified to eliminate expression of all PENK peptides or to tune MENK and/or LENK levels by selectively modifying cleavage sites. For example, one cleavage site may be left such that only one copy of MENK is produced. Thus, the effects of no expression of PENK peptides is avoided, however anti-tumor immunity is enhanced. Any feature described below may further be modified to alter PENK expression or activity.

The PENK (Proenkephalin-A) gene encodes a 267 residue protein

(SEQ ID NO: 23)

| | |
|---|---|
| 1 | marfltlctw llllgpglla tvraecsqdc atcsyrlvrp |
| | adinflacvm ecegklpslk |
| 61 | iwetckellq lskpelpqdg tstlrenskp eeshllakry |
| | ggfmkryggf mkkmdelypm |
| 121 | epeeeangse ilakryggfm kkdaeeddsl anssdllkel |
| | letgdnrers hhqdgsdnee |
| 181 | evskryggfm rglkrspqle deakelqkry ggfmrrvgrp |
| | ewwmdyqkry ggflkrfaea |
| 241 | lpsdeegesy skevpemekr yggfmrf |

Cleavage sites (CTSL): 111..112, 112..113, 133..134, 214..215, 215..216, 218..219

Processed active peptides: 25..97 (Synenkephalin), 100..104 (MENK), 107..111 (MENK), 114..133 (PENK), 136..140 (MENK), 143..183 (PENK), 186..193 (MENK), 210..214 (MENK), 230..234 (LENK), 237..258 (PENK), 261..267 (MENK)

1..24 “Signal”

25..70 “Opiods_neuropep”

83 “Variant” T->N (in dbSNP:rs11998459)

196..207 “Propeptide”

217..227 “Propeptide”

247 “Variant” G->D (in dbSNP:rs1800567)

251 “phosphorylation”

### OGFr Example Modifications

OGFr-specific mutations may be introduced using the gene editing systems described above. 1n particular in generating T cells that are resistant to dysfunction for use in adoptive cell transfer (described further below). In one example embodiment, the NLS of OGFr is modified. such that OGFr cannot localize to the nucleus after binding a PENK peptide. Any feature described below may further be modified to alter OGFr expression or activity (see, also UniProtKB/Swiss-Prot: Q9NZT2.3).

The OGFR gene encodes a 677 residue protein:

(SEQ ID NO: 24)

| | |
|---|---|
| 1 | mddpdcdstw eedeedaeda ededcedgea agardadagd |
| | edeeseepra arpssfqsrm |
| 61 | tgsrnwratr dmcryrhnyp dlverdcngd tpnlsfyrne |
| | irflpngcfi edilqnwtdn |
| 121 | ydllednhsy iqwlfplrep gvnwhakplt lrevevfkss |
| | qeiqerlvra yelmlgfygi |
| 181 | rledrgtgtv graqnyqkrf qnlnwrshnn lritrilksl |
| | gelglehfqa plvrffleet |
| 241 | lvrrelpgvr qsaldyfmfa vrcrhqrrql vhfawehfrp |
| | rckfvwgpqd klrrfkpssl |
| 301 | phplegsrkv eeegspgdpd heastqgrtc gpehskgggr |
| | vdegpqprsv epqdagpler |
| 361 | sqgdeagghg edrpeplspk eskkrklels rreqpptepg |
| | pqsaseveki alnlegcals |
| 421 | qgslrtgtqe vggqdpgeav qperqplgar vadkvrkrrk |
| | vdegagdsaa vasggaqtla |
| 481 | lagspapsgh pkaghsengv eedtegrtgp kegtpgspse |
| | tpgpspagpa gdepaespse |
| 541 | tpgprpagpa gdepaespse tpgprpagpa gdepaespse |
| | tpgpspagpt rdepaespse |
| 601 | tpgprpagpa gdepaespse tpgprpagpa gdepaespse |
| | tpgpspagpt rdepakagea |
| 661 | aelqdaeves saksgkp |

267..283 Bipartite nuclear localization signal

### Modified Immune Cells and Uses for Enhancing Anti-Tumor Immunity

In one aspect, embodiments disclosed herein are directed is isolated immune cells that have been modified to eliminate expression or activity of one of the ligand-receptor pairs disclosed herein. In one example embodiments, the immune cell is modified to eliminate expression or activity of OGFR. In another example embodiment, the immune cell is modified to eliminate expression of PENK or modify expression of PENK such that the resulting gene product is not processed into one of PENK's proteolytic and biologically active derivatives. The isolated immune cell may be an autologous immune cell isolated from a subject too be treated, i.e. a subject in need of an enhanced anti-tumor immune response. In other example embodiments, the isolated immune cell may be from an allogenic donor. In one example embodiment, the immune cell in any of the aforementioned example embodiments, may be a CD8 T cell. In on example embodiment, the CD8 T cell may express a chimeric antigen receptor (CAR) or T cell receptor (TCR) for a particular tumor antigen.

The modified immune cells may be generated using the gene editing systems described herein. Clinical application of CRISPR-Cas9 gene-edited T cells is generally safe and feasible (see, e.g., Lu Y, Xue J, Deng T, et al. Safety and feasibility of CRISPR-edited T cells in patients with refractory non-small-cell lung cancer [published correction appears in Nat Med. 2020 July; 26(7):1149]. Nat Med. 2020; 26(5):732-740; Lacey S F, Fraietta J A. First Trial of CRISPR-Edited T cells in Lung Cancer. Trends Mol Med. 2020; 26(8):713-715; and Zhang X, Cheng C, Sun W, Wang H. Engineering T Cells Using CRISPR/Cas9 for Cancer Therapy. Methods Mol Biol. 2020; 2115:419-433). Immune cells can also be edited ex vivo using Zn Finger proteins (see, e.g., Perez E E, Wang J, Miller J C, et al. Establishment of HIV-1 resistance in CD4+ T cells by genome editing using zinc-finger nucleases. Nat Biotechnol. 2008; 26(7):808-816). PENK modifications to reduce or eliminate PENK expression or peptide generation in T cells may include any modifications as described above (e.g., gene knockout or altering one or more cleavage sites to reduce active MENK and/or LENK). OGFr modifications to reduce or eliminate OGFr expression or activity (e.g., gene knockout or altering the NLS or binding site for MENK).

### Adoptive Cell Transfer

In certain embodiments, the methods of the present invention may be used to modulate interacting immune cells ex vivo for transfer into a subject. In certain embodiments, modulating one or more identified therapeutic targets in an immune cell shifts the immune cell to be resistant to dysfunction or have increased effector function (e.g., OGFr, PENK, NRP1, CRTAM). Such immune cells may be used to increase the effectiveness of adoptive cell transfer. In certain embodiments, immune cells are modulated using a genetic modifying agent, antibody or small molecule, described further herein. In certain embodiments, OGFr is decreased or eliminated in T cells for adoptive cell transfer. The modified T cells are applicable for T cells specific for any tumor antigen, such as tumor antigens described further herein. In certain embodiments, OGFr is decreased or eliminated in any immune cell that expresses OGFr, for example, natural killer cells (NK). In certain embodiments, PENK is decreased or eliminated in T cells for adoptive cell transfer. In certain embodiments, PENK is decreased or eliminated in any immune cell that expresses PENK, for example, natural killer cells (NK).

As used herein, “ACT”, “adoptive cell therapy” and “adoptive cell transfer” may be used interchangeably. In certain embodiments, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 June; 24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CART Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostate; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDGlcp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDGlcp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B 1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAXS); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukaemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In certain embodiments, an antigen to be targeted in adoptive cell therapy (such as particularly CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a neoantigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (Dl), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CIVIL), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR α and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Patent Publication WO9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8a hinge domain and a CD8a transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI 1a-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon R1b), CD79a, CD79b, Fc gamma RIM, DAP10, and DAP12. In certain preferred embodiments, the primary signaling domain comprises a functional signaling domain of CD3ζ or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3ζ chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139 (sequence version 1, 2 or 3): IEVMYPPPYLDNEKSNGTIIHVKGKHLCPSPLFPGPSKPFWVLVVVGGVLACYSLLVTVA FIIFWVRSKRSRLLHSDYMNMTPRRPGPTRKHYQPYAPPRDFAAYRS)) (SEQ. I.D. No. 25). Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-ζ molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY (SEQ. I.D. No. 26) and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY (SEQ ID NO: 26) and continuing all the way to the carboxy-terminus of the protein. The sequence is reproduced herein: IEVMYPPPYLDNEKSNGTIIHVKGKHLCPSPLFPGPSKPFWVLVVVGGVLACYSLLVTVA FIIFWVRSKRSRLLHSDYMNMTPRRPGPTRKHYQPYAPPRDFAAYRS (SEQ ID NO: 27). Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in International Patent Publication No. WO2015187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signalling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FcεRI gamma chain; or CD28-FcεRI gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signalling domain as set forth in Table 1 of WO2015187528. Preferably, the antigen is CD19, more preferably the antigen-binding element is an anti-CD19 scFv, even more preferably the mouse or human anti-CD19 scFv as described in Example 1 of WO2015187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in International Patent Publication No. WO2012058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20(1):55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am. J. Pathol. 1995; 147: 1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CART cells of this kind may for example be used in animal models, for example to treat tumor xenografts.

In certain embodiments, ACT includes co-transferring CD4+Th1 cells and CD8+ CTLs to induce a synergistic antitumour response (see, e.g., Li et al., Adoptive cell therapy with CD4+T helper 1 cells and CD8+ cytotoxic T cells enhances complete rejection of an established tumour, leading to generation of endogenous memory responses to non-targeted tumour epitopes. Clin Transl Immunology. 2017 October; 6(10): e160).

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4⁺ T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In vivo, Cell Stem Cell 22,1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells or population of cells, may be made resistant to at least one immunosuppressive agent due to the inactivation of a gene encoding a receptor for such immunosuppressive agent. In certain embodiments, the immunosuppressive treatment provides for the selection and expansion of the immunoresponsive T cells within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e. intracavity delivery) or directly into a tumor prior to resection (i.e. intratumoral delivery). In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23(9):2255-2266. doi: 10.1158/1078-0432.CCR-16-1300. Epub 2016 Nov. 4; Qasim et al., 2017, Molecular remission of infant B-ALL after infusion of universal TALEN gene-edited CART cells, Sci Transl Med. 2017 Jan. 25; 9(374); Legut, et al., 2018, CRISPR-mediated TCR replacement generates superior anticancer transgenic T cells. Blood, 131(3), 311-322; and Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see PCT Patent Publications: WO2013176915, WO2014059173, WO2014172606, WO2014184744, and WO2014191128).

In certain embodiments, editing may result in inactivation of a gene. By inactivating a gene, it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells (such as by CRISPR/Cas), particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, a and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells (such as by CRISPR/Cas), particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells (such as by CRISPR/Cas), particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1) (see, e.g., Rupp L J, Schumann K, Roybal K T, et al. CRISPR/Cas9-mediated PD-1 disruption enhances anti-tumor efficacy of human chimeric antigen receptor T cells. Sci Rep. 2017; 7(1):737). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

International Patent Publication No. WO2014172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, International Patent Publication No. WO2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as CRISPR, TALEN or ZFN) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CART cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, such as by CRISPR, ZNF or TALEN (for example, as described in WO201704916).

In certain embodiments, editing of cells (such as by CRISPR/Cas), particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACT), and B-cell activating factor receptor (BAFF-R) (for example, as described in International Patent Publication Nos. WO2016011210 and WO2017011804).

In certain embodiments, editing of cells (such as by CRISPR/Cas), particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In certain embodiments, a cell may be multiply edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L 1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MEW tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MEW class I may be evaluated indirectly by monitoring the ability to promote incorporation of ¹²⁵I labeled β2-microglobulin (β2m) into MEW class I/β2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™ BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR, may be manufactured as described in International Patent Publication No. WO2015120096, by a method comprising enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO2015120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in WO2017070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in WO2016191756 comprising administering to the patient a dose of cyclophosphamide between 200 mg/m2/day and 2000 mg/m2/day and a dose of fludarabine between 20 mg/m2/day and 900 mg/m²/day.

### Additional Therapies

### Combination Therapies

Aspects of the invention involve modifying the therapy within a standard of care based on the detection of any of the biomarkers as described herein. For example, an antagonist of opioid signaling, e.g., OGFr signaling is administered in combination with an immunotherapy, such as adoptive cell transfer or checkpoint blockade therapy. In certain embodiments, reducing opioid signaling enhances the activity of adoptive cell transfer (e.g., by preventing transferred cells from becoming dysfunctional) or further enhances anti-tumor immunity in combination with checkpoint blockade therapy. In one embodiment, therapy comprising an agent is administered within a standard of care where addition of the agent is synergistic within the steps of the standard of care. In one embodiment, the agent targets and/or shifts a tumor to an immunotherapy responder phenotype. In one embodiment, the agent inhibits expression or activity of one or more transcription factors capable of regulating a gene program. In one embodiment, the agent targets tumor cells expressing a gene program. The term “standard of care” as used herein refers to the current treatment that is accepted by medical experts as a proper treatment for a certain type of disease and that is widely used by healthcare professionals. Standard of care is also called best practice, standard medical care, and standard therapy. Standards of care for cancer generally include surgery, lymph node removal, radiation, chemotherapy, targeted therapies, antibodies targeting the tumor, and immunotherapy. Immunotherapy can include checkpoint blockers (CBP), chimeric antigen receptors (CARs), and adoptive T-cell therapy. The standards of care for the most common cancers can be found on the website of National Cancer Institute (www.cancer.gov/cancertopics). A treatment clinical trial is a research study meant to help improve current treatments or obtain information on new treatments for patients with cancer. When clinical trials show that a new treatment is better than the standard treatment, the new treatment may be considered the new standard treatment.

The term “Adjuvant therapy” as used herein refers to any treatment given after primary therapy to increase the chance of long-term disease-free survival. The term “Neoadjuvant therapy” as used herein refers to any treatment given before primary therapy. The term “Primary therapy” as used herein refers to the main treatment used to reduce or eliminate the cancer. In certain embodiments, an agent that shifts a tumor to a responder phenotype are provided as a neoadjuvant before CPB therapy.

### Immunotherapy

In certain embodiments, the one or more agents described herein are administered to enhance the effectiveness of an immunotherapy, e.g., by preventing suppression of an immune response. Immunotherapy can include checkpoint blockers (CBP), chimeric antigen receptors (CARs), and adoptive T-cell therapy.

Antibodies that block the activity of checkpoint receptors, including CTLA-4, PD-1, Tim-3, Lag-3, and TIGIT, either alone or in combination, have been associated with improved effector CD8⁺ T cell responses in multiple pre-clinical cancer models (Johnston et al., 2014. The immunoreceptor TIGIT regulates antitumor and antiviral CD8(+) T cell effector function. Cancer cell 26, 923-937; Ngiow et al., 2011. Anti-TIM3 antibody promotes T cell IFN-gamma-mediated antitumor immunity and suppresses established tumors. Cancer research 71, 3540-3551; Sakuishi et al., 2010. Targeting Tim-3 and PD-1 pathways to reverse T cell exhaustion and restore anti-tumor immunity. The Journal of experimental medicine 207, 2187-2194; and Woo et al., 2012. Immune inhibitory molecules LAG-3 and PD-1 synergistically regulate T-cell function to promote tumoral immune escape. Cancer research 72, 917-927). Similarly, blockade of CTLA-4 and PD-1 in patients (Brahmer et al., 2012. Safety and activity of anti-PD-L1 antibody in patients with advanced cancer. The New England journal of medicine 366, 2455-2465; Hodi et al., 2010. Improved survival with ipilimumab in patients with metastatic melanoma. The New England journal of medicine 363, 711-723; Schadendorf et al., 2015. Pooled Analysis of Long-Term Survival Data From Phase II and Phase III Trials of Ipilimumab in Unresectable or Metastatic Melanoma. Journal of clinical oncology: official journal of the American Society of Clinical Oncology 33, 1889-1894; Topalian et al., 2012. Safety, activity, and immune correlates of anti-PD-1 antibody in cancer. The New England journal of medicine 366, 2443-2454; and Wolchok et al., 2017. Overall Survival with Combined Nivolumab and Ipilimumab in Advanced Melanoma. The New England journal of medicine 377, 1345-1356) has shown increased frequencies of proliferating T cells, often with specificity for tumor antigens, as well as increased CD8⁺ T cell effector function (Ayers et al., 2017. IFN-gamma-related mRNA profile predicts clinical response to PD-1 blockade. The Journal of clinical investigation 127, 2930-2940; Das et al., 2015. Combination therapy with anti-CTLA-4 and anti-PD-1 leads to distinct immunologic changes in vivo. Journal of immunology 194, 950-959; Gubin et al., 2014. Checkpoint blockade cancer immunotherapy targets tumour-specific mutant antigens. Nature 515, 577-581; Huang et al., 2017. T-cell invigoration to tumour burden ratio associated with anti-PD-1 response. Nature 545, 60-65; Kamphorst et al., 2017. Proliferation of PD-1+CD8 T cells in peripheral blood after PD-1-targeted therapy in lung cancer patients. Proceedings of the National Academy of Sciences of the United States of America 114, 4993-4998; Kvistborg et al., 2014. Anti-CTLA-4 therapy broadens the melanoma-reactive CD8+ T cell response. Science translational medicine 6, 254ra128; van Rooij et al., 2013. Tumor exome analysis reveals neoantigen-specific T-cell reactivity in an ipilimumab-responsive melanoma. Journal of clinical oncology: official journal of the American Society of Clinical Oncology 31, e439-442; and Yuan et al., 2008. CTLA-4 blockade enhances polyfunctional NY-ESO-1 specific T cell responses in metastatic melanoma patients with clinical benefit. Proceedings of the National Academy of Sciences of the United States of America 105, 20410-20415). Accordingly, the success of checkpoint receptor blockade has been attributed to the binding of blocking antibodies to checkpoint receptors expressed on dysfunctional CD8⁺ T cells and restoring effector function in these cells. The check point blockade therapy may be an inhibitor of any check point protein described herein. The checkpoint blockade therapy may comprise anti-TIM3, anti-CTLA4, anti-PD-L1, anti-PD1, anti-TIGIT, anti-LAG3, or combinations thereof. Anti-PD1 antibodies are disclosed in U.S. Pat. No. 8,735,553. Antibodies to LAG-3 are disclosed in U.S. Pat. No. 9,132,281. Anti-CTLA4 antibodies are disclosed in U.S. Pat. Nos. 9,327,014; 9,320,811; and 9,062,111. Specific check point inhibitors include, but are not limited to anti-CTLA4 antibodies (e.g., Ipilimumab and tremelimumab), anti-PD-1 antibodies (e.g., Nivolumab, Pembrolizumab), and anti-PD-L1 antibodies (e.g., Atezolizumab).

In certain embodiments, non-opioid pain medication is administered to a subject being treated with a therapy described herein or being treated with an immunotherapy. In certain embodiments, opioids are not administered to a subject having cancer, in particular not in combination with an immunotherapy.

### Additional Therapeutic Targets

Applicants have identified additional receptor-ligand interactions (see, Table 1 and FIG. 5) that may targeted using the same exemplary embodiments described above and as applied to PENK-OGFR. In one example embodiment, a VEGFB-NRP1 interaction is inhibited by an antagonist to prevent VEGFB expressing tumors from interacting with NRP1 expressing T cells. In one example embodiment, a CADM1-CRTAM interaction is inhibited by an antagonist to prevent CADM1 expressing tumors from interacting with CRTAM expressing T cells. In certain embodiments, T cell dysfunction is delayed or prevented by blocking communication with the tumor.

In one example embodiment, a TFPI—SDC4 interaction is inhibited by an antagonist to prevent TFPI expressing tumors from interacting with SDC4 expressing Treg cells. In one example embodiment, a MFGE8-ITGAV interaction is inhibited by an antagonist to prevent MFGE8 expressing tumors from interacting with ITGAV expressing Treg cells. In one example embodiment, a ADAM12-SDC4 interaction is inhibited by an antagonist to prevent ADAM12 expressing tumors from interacting with SDC4 expressing Treg cells. In one example embodiment, a CYR61-ITGAV interaction is inhibited by an antagonist to prevent CYR61 expressing tumors from interacting with ITGAV expressing Treg cells. In certain embodiments, Treg activation is delayed or prevented by blocking communication with the tumor.

Applicants have identified additional genes expressed in the T and non-T cell types that change during tumor progression, such as with time and tumor size. In certain embodiments, cell types capable of signaling other cell types in the TME are positively or negatively correlated with time and size. In certain embodiments, signaling molecules expressed in specific cell types are positively or negatively correlated with time and size. In certain embodiments, signaling from the cell types suppress or enhance anti-tumor immunity in the TME. In certain embodiments, the cell types or genes can be detected or modified. Applicants identified differentially expressed genes associated with tumor progression (time) in TME cells (Table 2) and differentially expressed genes associated with tumor size in TME cells (Table 3). PENK was identified as a highly scoring gene associated with time and size and specifically expressed in dysfunctional T cells (clusters T_4 and T_7) (see, Tables 2 and 3). PENK signaling in T cell dysfunction was validated in vitro and in vivo. The other genes correlated with tumor time and size may be targeted using the same exemplary embodiments described above and as applied to PENK-OGFR.

### Pharmaceutical Formulations and Administration

Also described herein are pharmaceutical formulations that can contain an amount, effective amount, and/or least effective amount, and/or therapeutically effective amount of one or more of the small molecules, RNAi therapeutics, vectors, recombinant polypeptides, gene editing systems, conjugated-antibodies, or engineered cells as described above, or a combination thereof (which are also referred to as the primary active agent or ingredient elsewhere herein) described in greater detail elsewhere herein a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intraci sternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having a Type-2 Diabetes or a symptom thereof. In some embodiments, the subject in need thereof has or is suspected of having, a metabolic disease or disorder, insulin resistance, or glucose intolerance, or a combination thereof. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects.

The effective amount, least effective amount, and/or therapeutically effective amount of the primary and optional secondary active agent described elsewhere herein contained in the pharmaceutical formulation can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, mg, or g or be any numerical value with any of these ranges.

In some embodiments, the effective amount, least effective amount, and/or therapeutically effective amount can be an effective concentration, least effective concentration, and/or therapeutically effective concentration, which can each range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, mM, or M or be any numerical value with any of these ranges.

In other embodiments, the effective amount, least effective amount, and/or therapeutically effective amount of the primary and optional secondary active agent can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In some embodiments, the primary and/or the optional secondary active agent present in the pharmaceutical formulation can range from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation.

In some embodiments where a cell population is present in the pharmaceutical formulation (e.g., as a primary and/or or secondary active agent), the effective amount of cells can range from about 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL.

In some embodiments, the amount or effective amount, particularly where an infective particle is being delivered (e.g. a virus particle having the primary or secondary agent as a cargo), the effective amount of virus particles can be expressed as a titer (plaque forming units per unit of volume) or as a MOI (multiplicity of infection). In some embodiments, the effective amount can be 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more.

In some embodiments, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total secondary active agent in the pharmaceutical formulation. In additional embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g. micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, manitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Administration of the Pharmaceutical Formulations

The pharmaceutical formulations or dosage forms thereof described herein can be administered one or more times hourly, daily, monthly, or yearly (e.g. 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g. 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g. within seconds or just a few minutes apart), where the intent is that the formulations be administered together at the same time.

### Viral Vector Formulation, Dosage, and Delivery

Compositions of the invention may be formulated for delivery to human subjects, as well as to animals for veterinary purposes (e.g. livestock (cattle, pigs, others)), and other non-human mammalian subjects. The dosage of the formulation can be measured or calculated as viral particles or as genome copies (“GC”)/viral genomes (“vg”). Any method known in the art can be used to determine the genome copy (GC) number of the viral compositions of the invention. In one example embodiment, the viral compositions can be formulated in dosage units to contain an amount of viral vectors that is in the range of about 1.0×10⁹ GC to about 1.0×10¹⁵ GC (to treat an average subject of 70 kg in body weight), and preferably 1.0×10¹² GC to 1.0×10¹⁴ GC for a human patient. Preferably, the dose of virus in the formulation is 1.0×10⁹ GC, 5.0×10⁹ GC, 1.0×10¹⁰ GC, 5.0×10¹⁰ GC, 1.0×10¹¹ GC, 5.0×10¹¹ GC, 1.0×10¹² GC, 5.0×10¹² GC, or 1.0×10¹³ GC, 5.0×10¹³ GC, 1.0×10¹⁴ GC, 5.0×10¹⁴ GC, or 1 0.0×10¹⁵ GC.

The viral vectors can be formulated in a conventional manner using one or more physiologically acceptable carriers or excipients. The viral vectors may be formulated for parenteral administration by injection (e.g. by bolus injection or continuous infusion). Formulations for injection may be presented in unit dosage form (e.g. in ampoules or in multi-dose containers) with an added preservative. The viral compositions may take such forms as suspensions, solutions, or emulsions in oily or aqueous vehicles, and may contain formulatory agents such as suspending, stabilizing, or dispersing agents. Liquid preparations of the viral vector formulations may be prepared by conventional means with pharmaceutically acceptable additives such as suspending agents (e.g. sorbitol syrup, cellulose derivatives or hydrogenated edible fats), emulsifying agents (e.g. lecithin or acacia), non-aqueous vehicles (e.g. almond oil, oily esters, ethyl alcohol or fractionated vegetable oils), and preservatives (e.g. methyl or propyl-p-hydroxybenzoates or sorbic acid). The preparations may also contain buffer salts. Alternatively, the compositions may be in powder form for constitution with a suitable vehicle (e.g. sterile pyrogen-free water) before use.

### Recombinant Protein Formulation, Dosage, and Delivery

In one example embodiment, virus like particles (VLPs) are used to facilitate intracellular recombinant protein therapy (see, e.g., WO2020252455A1, U.S. Ser. No. 10/577,397B2). In certain embodiments, VLPs include a Gag-competitive PENK peptide fusion protein. The Gag-competitive PENK peptide fusion protein may include a matrix protein, a capsid protein, and/or a nucleocapsid protein covalently linked to the competitive PENK peptide. In certain embodiments, the VLPs include a membrane comprising a phospholipid bilayer with one or more human endogenous retrovirus (HERV) derived ENV/glycoprotein(s) on the external side; a HERV-derived GAG protein in the VLP core, and a competitive PENK peptide fusion protein on the inside of the membrane, wherein the competitive PENK peptide is fused to a human-endogenous GAG or other plasma membrane recruitment domain (see, e.g., WO2020252455A1). Fusion proteins can be obtained using standard recombinant protein technology.

In one example embodiment, cell-penetrating peptides (CPPs) are used to facilitate intracellular recombinant protein therapy (see, e.g., Dinca A, Chien W-M, Chin M T. Intracellular Delivery of Proteins with Cell-Penetrating Peptides for Therapeutic Uses in Human Disease. International Journal of Molecular Sciences. 2016; 17(2):263). In certain embodiments, cell-penetrating peptides can be conjugated to the competitive PENK peptide, for example, using standard recombinant protein technology. In certain embodiments, cell-penetrating peptides can be concurrently delivered with recombinant competitive PENK peptide.

In one example embodiment, nanocarriers are used to facilitate intracellular recombinant protein therapy (see, e.g., Lee Y W, Luther D C, Kretzmann J A, Burden A, Jeon T, Zhai S, Rotello V M. Protein Delivery into the Cell Cytosol using Non-Viral Nanocarriers. Theranostics 2019; 9(11):3280-3292). Non-limiting nanocarriers include, but are not limited to nanoparticles (e.g., silica, gold), polymers, lipid based (e.g., cationic lipid within a polymer shell, lipid-like nanoparticles).

The pharmaceutical composition of the invention may be administered locally or systemically. In a preferred embodiment, the pharmaceutical composition is administered near the tissue whose cells are to be transduced. In a particular embodiment, the pharmaceutical composition of the invention is administered locally to the tumor tissue. In another preferred embodiment, the pharmaceutical composition of the invention is administered systemically.

The “adeno-associated virus” (AAV) can be formulated with a physiologically acceptable carrier for use in gene transfer and gene therapy applications. The dosage of the formulation can be measured or calculated as viral particles or as genome copies (“GC”)/viral genomes (“vg”). Any method known in the art can be used to determine the genome copy (GC) number of the viral compositions of the invention. One method for performing AAV GC number titration is as follows: purified AAV vector samples are first treated with DNase to eliminate un-encapsulated AAV genome DNA or contaminating plasmid DNA from the production process. The DNase resistant particles are then subjected to heat treatment to release the genome from the capsid. The released genomes are then quantitated by real-time PCR using primer/probe sets targeting specific region of the viral genome.

In any of the described methods the one or more vectors may be comprised in a delivery system. In any of the described methods the vectors may be delivered via liposomes, particles (e.g., nanoparticles), exosomes, microvesicles, a gene-gun. In any of the described methods viral vectors may be delivered by transduction of viral particles. The delivery systems may be administered systemically or by localized administration (e.g., direct injection). The term “systemically administered” and “systemic administration”, as used herein, means that the polynucleotides, vectors, polypeptides, or pharmaceutical compositions of the invention are administered to a subject in a non-localized manner. The systemic administration of the polynucleotides, vectors, polypeptides, or pharmaceutical compositions of the invention may reach several organs or tissues throughout the body of the subject or may reach specific organs or tissues of the subject. For example, the intravenous administration of a pharmaceutical composition of the invention may result in the transduction of more than one tissue or organ in a subject. The term “transduce” or “transduction”, as used herein, refers to the process whereby a foreign nucleotide sequence is introduced into a cell via a viral vector. The term “transfection”, as used herein, refers to the introduction of DNA into a recipient eukaryotic cell.

Recombinant protein compositions described herein may be administered systemically (e.g., intravenously) or administered locally to tumor tissue (e.g., injection). In preferred embodiments, the recombinant protein compositions are administered with an appropriate carrier to be administered to a mammal, especially a human, preferably a pharmaceutically acceptable composition. A “pharmaceutically acceptable composition” refers to a non-toxic semisolid, liquid, or aerosolized filler, diluent, encapsulating material, colloidal suspension or formulation auxiliary of any type. Preferably, this composition is suitable for injection. These may be in particular isotonic, sterile, saline solutions (monosodium or disodium phosphate, sodium, potassium, calcium or magnesium chloride and similar solutions or mixtures of such salts), or dry, especially freeze-dried compositions which upon addition, depending on the case, of sterilized water or physiological saline, permit the constitution of injectable solutions.

### CRISPR-Cas Delivery

The CRISPR-Cas systems disclosed herein may be delivered using vectors comprising polynucleotides encoding the Cas polypeptide and the guide molecule. For HDR based embodiments, the donor template may also be encoded on a vector. Vectors, dosages, and adipocyte-specific configurations suitable for delivery of these components include those discussed above.

The vector(s) can include regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well-established cloning protocols and the teachings in this disclosure one skilled in the art can readily practice the invention as to the RNA(s) for a suitable exemplary vector such as AAV, and a suitable promoter such as the U6 promoter. For example, the packaging limit of AAV is ˜4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector is to express an array of promoter-RNAs separated by cleavable sequences in the intron of a coding sequence or gene; and, in this instance, it is advantageous to use a polymerase II promoter, which can have increased expression and enable the transcription of long RNA in a tissue specific manner. (see, e.g., Chung K H, Hart C C, Al-Bassam S, et al. Polycistronic RNA polymerase II expression vectors for RNA interference based on BIC/miR-155. Nucleic Acids Res. 2006; 34(7):e53). In an advantageous embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters, especially as to the numbers of RNAs or guides discussed herein, without any undue experimentation.

The Cas polypeptide and guide molecule (and donor) may also be delivered as a pre-formed ribonucleoprotein complex (RNP). Delivery methods for delivery RNPs include virus like particles, cell-penetrating peptides, and nanocarriers discussed above.

Delivery mechanisms for CRISPRa systems include virus like particles, cell-penetrating peptides, and nanocarriers discussed above for CRISPR-Cas systems.

### Base Editing Delivery

Base editing systems may deliver on one or more vectors encoding the Cas-nucleobase deaminase and guide sequence. Vector systems suitable for this purpose includes those discussed above. Alternatively, base editing systems may be delivered as pre-complex Ribonucleoprotein complex (RNP. Systems for delivering RNPs include the protein delivery systems: virus like particles; cell-penetrating peptides; and nanocarriers, discuss above.

A further example method for delivery of base-editing systems may include use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Methods of Monitoring Tumor Progression

In certain embodiments, tumor progression is monitored in a subject by detecting genes or cell types associated with tumor time or size (Tables 2 and 3). In certain embodiments, detection of one or more genes can be used to determine the effectiveness of a cancer treatment. In certain embodiments, PENK expression is detected and indicates dysfunctional immune cells or a suppressive immune state.

In certain embodiments, detection of interacting cell types indicates resistance to an immune response (Table 1). In certain embodiments, detection of interacting cells in the TME may indicate that the subject may be responsive to an immunotherapy. In one example embodiment, expression of receptors or ligands on tumor cells that can interact with immune cells in the TME indicates that the tumor can modulate the TME to suppress an anti-tumor immune response. In certain embodiments, a tumor sample, such as a tissue section can be obtained and stained for any of VEGFB, TFPI, MFGE8, ADAM12 and FBLN1.

### Detection of Biomarkers

In certain embodiments, the invention provides uses of the biomarkers for predicting risk for a certain phenotype. In certain embodiments, the invention provides uses of the biomarkers for selecting a treatment. In certain embodiments, a subject having a disease can be classified based on severity of the disease. In certain embodiments, a tumor is monitored for expression of a biomarker described herein. The invention provides biomarkers for the identification, diagnosis, prognosis and manipulation of disease phenotypes, for use in a variety of diagnostic and/or therapeutic indications. Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein.

The terms “diagnosis” and “monitoring” are commonplace and well-understood in medical practice. By means of further explanation and without limitation the term “diagnosis” generally refers to the process or act of recognizing, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The biomarkers of the present invention are useful in methods of identifying specific patient populations based on a detected level of expression, activity and/or function of one or more biomarkers. These biomarkers are also useful in monitoring subjects undergoing treatments and therapies for suitable or aberrant response(s) to determine efficaciousness of the treatment or therapy and for selecting or modifying therapies and treatments that would be efficacious in treating, delaying the progression of or otherwise ameliorating a symptom. The biomarkers provided herein are useful for selecting a group of patients at a specific state of a disease with accuracy that facilitates selection of treatments.

The term “monitoring” generally refers to the follow-up of a disease or a condition in a subject for any changes which may occur over time.

The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

Hence, the methods may rely on comparing the quantity of biomarkers, or gene or gene product signatures measured in samples from patients with reference values, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favorable or unfavorable prognoses for such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterized by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value >second value; or decrease: first value <second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥λ% or ≥85% or ≥90% or ≥95% or even ≥00% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

In one embodiment, the signature genes, biomarkers, and/or cells expressing biomarkers may be detected or isolated by immunofluorescence, immunohistochemistry (IHC), fluorescence activated cell sorting (FACS), mass spectrometry (MS), mass cytometry (CyTOF), sequencing, WGS (described herein), WES (described herein), RNA-seq, single cell RNA-seq (described herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. Detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25). In certain embodiments, cancer is diagnosed, prognosed, or monitored. For example, a tissue sample may be obtained and analyzed for specific cell markers (IHC) or specific transcripts (e.g., RNA-FISH). In one embodiment, tumor cells are stained for cell subtype specific signature genes. In one embodiment, the cells are fixed. In another embodiment, the cells are formalin fixed and paraffin embedded. Not being bound by a theory, the presence of the tumor subtypes indicate outcome and personalized treatments.

The present invention also may comprise a kit with a detection reagent that binds to one or more biomarkers or can be used to detect one or more biomarkers.

### MS Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab)₂ fragment, a single chain antibody fragment, an Fv fragment, a single chain Fv fragment, a nucleic acid, a lectin, a ligand-binding receptor, affibodies, nanobodies, ankyrins, domain antibodies, alternative antibody scaffolds (e.g. diabodies etc.) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65 C for 4 hours followed by washes at 25° C. in low stringency wash buffer (1×SSC plus 0.2% SDS) followed by 10 minutes at 25° C. in high stringency wash buffer (0.1 SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992).

In certain embodiments, a subject can be categorized based on signature genes or gene programs expressed by a tissue sample obtained from the subject. In certain embodiments, the tissue sample is analyzed by bulk sequencing. In certain embodiments, subtypes can be determined by determining the percentage of specific cell subtypes expressing the identified interacting genetic variants in the sample that contribute to the phenotype. In certain embodiments, gene expression associated with the cells are determined from bulk sequencing reads by deconvolution of the sample. For example, deconvoluting bulk gene expression data obtained from a tumor containing both malignant and non-malignant cells can include defining the relative frequency of a set of cell types in the tumor from the bulk gene expression data using cell type specific gene expression (e.g., cell types may be T cells, fibroblasts, macrophages, mast cells, B/plasma cells, endothelial cells, myocytes and dendritic cells); and defining a linear relationship between the frequency of the non-malignant cell types and the expression of a set of genes, wherein the set of genes comprises genes highly expressed by malignant cells and at most two non-malignant cell types, wherein the set of genes are derived from gene expression analysis of single cells in the tumor or the same tumor type, and wherein the residual of the linear relationship defines the malignant cell-specific (MCS) expression profile (see, e.g., WO 2018/191553; and Puram et al., Cell. 2017 Dec. 14; 171(7):1611-1624.e24).

### Sequencing

In certain embodiments, sequencing is used to identify expression of genes or transcriptomes in single cells. In certain embodiments, sequencing comprises high-throughput (formerly “next-generation”) technologies to generate sequencing reads. Methods for constructing sequencing libraries are known in the art (see, e.g., Head et al., Library construction for next-generation sequencing: Overviews and challenges. Biotechniques. 2014; 56(2): 61-77). A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags. In certain embodiments, the library members (e.g., cDNA) may include sequencing adaptors that are compatible with use in, e.g., Illumina's reversible terminator method, long read nanopore sequencing, Roche's pyrosequencing method (454), Life Technologies' sequencing by ligation (the SOLiD platform) or Life Technologies' Ion Torrent platform. Examples of such methods are described in the following references: Margulies et al (Nature 2005 437: 376-80); Schneider and Dekker (Nat Biotechnol. 2012 Apr. 10; 30(4):326-8); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol. Biol. 2009; 553:79-108); Appleby et al (Methods Mol. Biol. 2009; 513:19-39); and Morozova et al (Genomics. 2008 92:255-64), which are incorporated by reference for the general descriptions of the methods and the particular steps of the methods, including all starting products, reagents, and final products for each of the steps.

As used herein the term “transcriptome” refers to the set of transcript molecules. In some embodiments, transcript refers to RNA molecules, e.g., messenger RNA (mRNA) molecules, small interfering RNA (siRNA) molecules, transfer RNA (tRNA) molecules, ribosomal RNA (rRNA) molecules, and complimentary sequences, e.g., cDNA molecules. In some embodiments, a transcriptome refers to a set of mRNA molecules. In some embodiments, a transcriptome refers to a set of cDNA molecules. In some embodiments, a transcriptome refers to one or more of mRNA molecules, siRNA molecules, tRNA molecules, rRNA molecules, in a sample, for example, a single cell or a population of cells. In some embodiments, a transcriptome refers to cDNA generated from one or more of mRNA molecules, siRNA molecules, tRNA molecules, rRNA molecules, in a sample, for example, a single cell or a population of cells. In some embodiments, a transcriptome refers to 25%, 50%, 55, 60, 65, 70, 75, 80, 85, 90, 91, 92, 93, 94, 95, 96, 97, 98, 99, 99.9, or 100% of transcripts from a single cell or a population of cells. In some embodiments, transcriptome not only refers to the species of transcripts, such as mRNA species, but also the amount of each species in the sample. In some embodiments, a transcriptome includes each mRNA molecule in the sample, such as all the mRNA molecules in a single cell.

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p666-673, 2012).

In certain embodiments, the present invention involves single cell RNA sequencing (scRNA-seq). In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq where the RNAs from different cells are tagged individually, allowing a single library to be created while retaining the cell identity of each read. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International Patent Application No. PCT/US2016/059239, published as WO2017164936 on Sep. 28, 2017; International Patent Application No. PCT/US2018/060860, published as WO/2019/094984 on May 16, 2019; International Patent Application No. PCT/US2019/055894, published as WO/2020/077236 on Apr. 16, 2020; and Drokhlyansky, et al., “The enteric nervous system of the human and mouse colon at a single-cell resolution,” bioRxiv 746743; doi: doi.org/10.1101/746743, which are herein incorporated by reference in their entirety.

### Screening for Modulating Agents

In certain embodiments, the invention provides for screening for therapeutic agents capable of altering the tumor microenvironment. In certain embodiments, agents capable of blocking or enhancing interacting cell types are screened. In certain embodiments, the method comprises: a) applying a candidate agent to a cell population comprising interacting cells; b) detecting modulation of one or more phenotypic aspects of the cell population by the candidate agent, thereby identifying the agent. The phenotypic aspects of the cell population that is modulated may be a gene signature specific to a cell type or cell phenotype or phenotype specific to a population of cells (e.g., an anti-tumor immune phenotype). In certain embodiments, steps can include administering candidate modulating agents to cells, detecting identified cell (sub)populations for changes in signatures, or identifying relative changes in cell (sub) populations which may comprise detecting relative abundance of particular gene signatures.

### Tumor Models

In certain embodiments, therapeutic agents are screened or validated in a tumor model. In certain embodiments, cell-cell interactions in a tumor are identified using single cell methods for one or more tumor samples. In certain embodiments, associations to tumor progression (e.g., time and size) are identified using single cell methods for one or more tumor samples. In certain embodiments, the tumor sample can be obtained from a tumor animal model. In certain embodiments, the tumor sample can be obtained from a mouse tumor model. Non-limiting examples of tumor mouse models include the CT26 colon carcinoma, MC38-Ova colon carcinoma and B16F10 melanoma models (see, e.g., Singer, M. et al. A Distinct Gene Module for Dysfunction Uncoupled from Activation in Tumor-Infiltrating T Cells. Cell 171, 1221-1223 (2017); and Kurtulus, S. et al. Checkpoint Blockade Immunotherapy Induces Dynamic Changes in PD-1(−)CD8(+) Tumor-Infiltrating T Cells. Immunity 50, 181-194 e186 (2019)).

In certain embodiments, the tumor sample can be obtained over a time course to capture interactions occurring at specific time points during tumor progression (see, e.g., International Patent Application Nos. PCT/US2018/053791, PCT/US2018/061812). The time course can be from 0 to 365 days after implantation of a tumor in the mouse model. Time points can be taken at any day within the time course. In preferred embodiments, the time course is for about 20 days and includes samples taken at about 5, 10, 15 and 20 days (e.g., day 11, 13, 15, 17 and 18).

In certain embodiments, the tumor sample can be obtained from one or more subjects suffering from cancer. The sample may be a fresh sample or frozen sample. Samples can be obtained from a subject over the course of treating the cancer. Samples can be obtained before and after treatment.

### Agents for Screening

In certain embodiments, agents are screened for inducing a phenotype. The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein.

The methods of phenotypic analysis can be utilized for evaluating environmental stress and/or state, for screening of chemical libraries, and to screen or identify structural, syntenic, genomic, and/or organism and species variations. For example, a culture of cells, can be exposed to an environmental stress, such as but not limited to heat shock, osmolarity, hypoxia, cold, oxidative stress, radiation, starvation, a chemical (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value. By exposing cells, or fractions thereof, tissues, or even whole animals, to different members of the chemical libraries, and performing the methods described herein, different members of a chemical library can be screened for their effect on immune phenotypes thereof simultaneously in a relatively short amount of time, for example using a high throughput method.

Aspects of the present disclosure relate to the correlation of an agent with the spatial proximity and/or epigenetic profile of the nucleic acids in a sample of cells. In some embodiments, the disclosed methods can be used to screen chemical libraries for agents that modulate chromatin architecture epigenetic profiles, and/or relationships thereof.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

In certain embodiments, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signatures or biological programs of the present invention may be used to screen for drugs that reduce the signature or biological program in cells as described herein. The signature or biological program may be used for GE-HTS. In certain embodiments, pharmacological screens may be used to identify drugs that are selectively toxic to cells having a signature.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, Cmap can be used to screen for small molecules capable of modulating a signature or biological program of the present invention in silico.

Further embodiments are illustrated in the following Examples which are given for illustrative purposes only and are not intended to limit the scope of the invention

### EXAMPLES

### Example 1—Single Cell Analysis of the Tumor Microenvironment Over Time

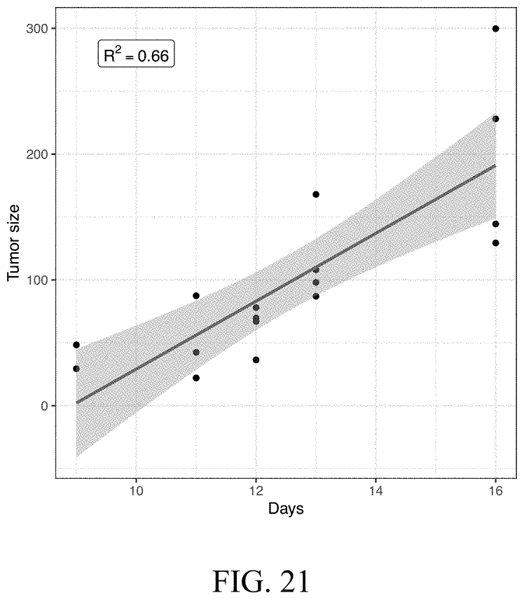

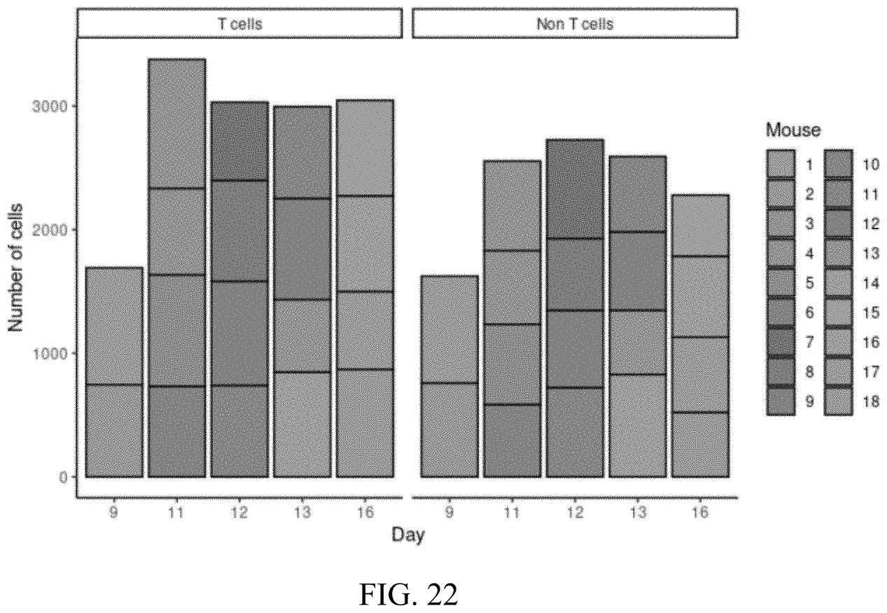

Applicants performed a time course study using B16 melanoma tumor mouse models. Applicants performed single cell sequencing on the tumor cells using the 10× genomics platform. The single cell transcriptomes were used to cluster the cell types and identify the cell types of each cluster (FIG. 1). Applicants used 18 mice that passed quality controls and cells were collected at different time points for single cell RNA-seq (FIG. 21-22).

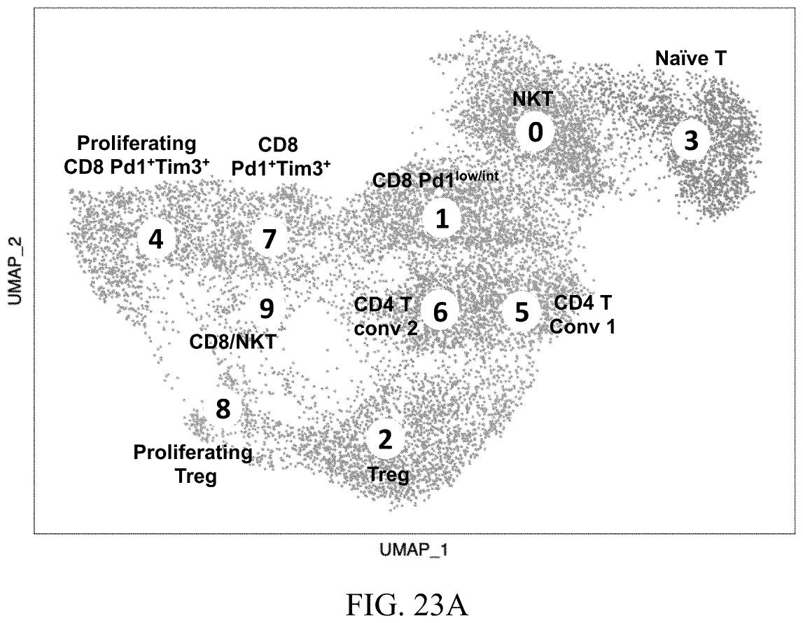

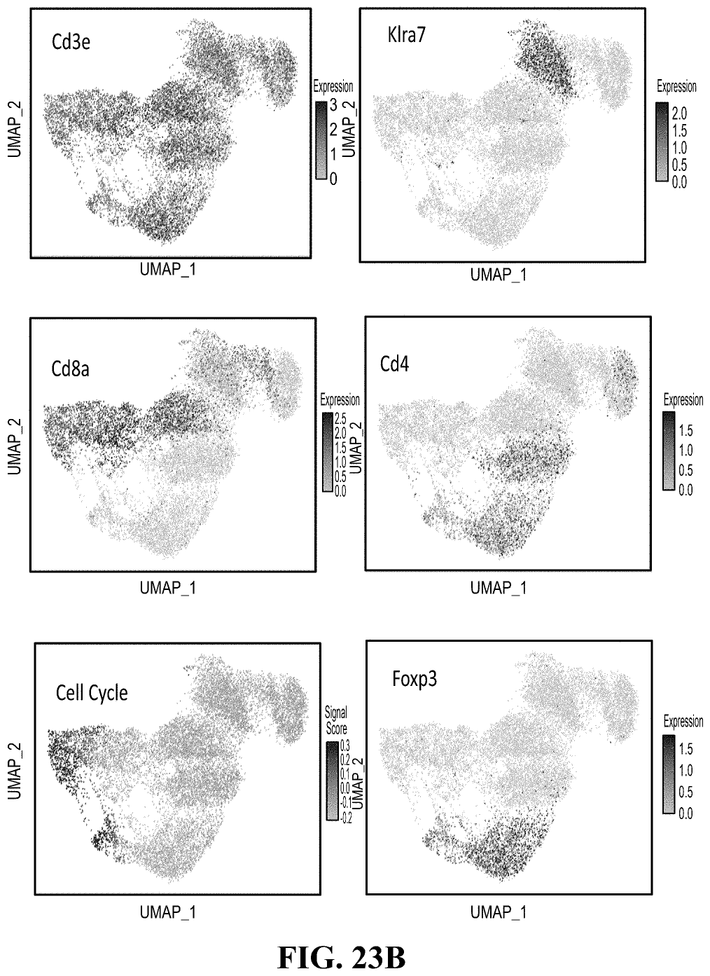

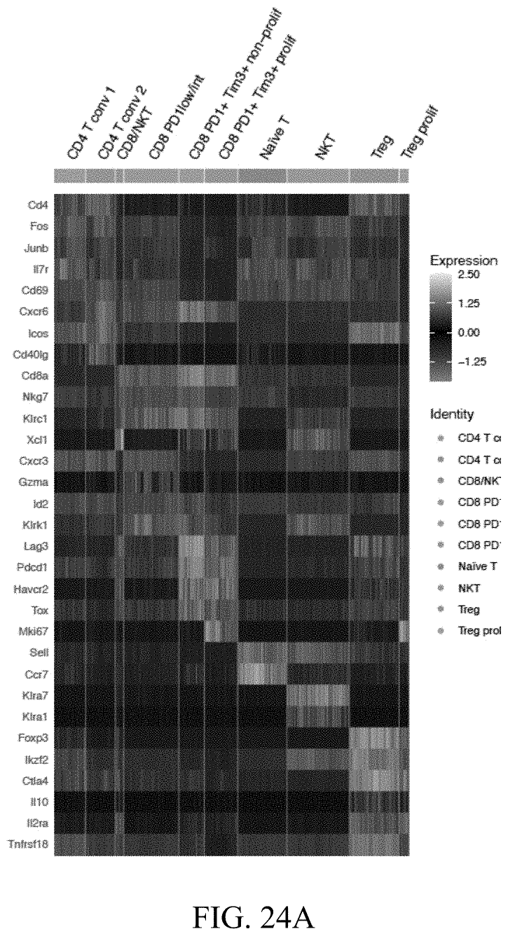

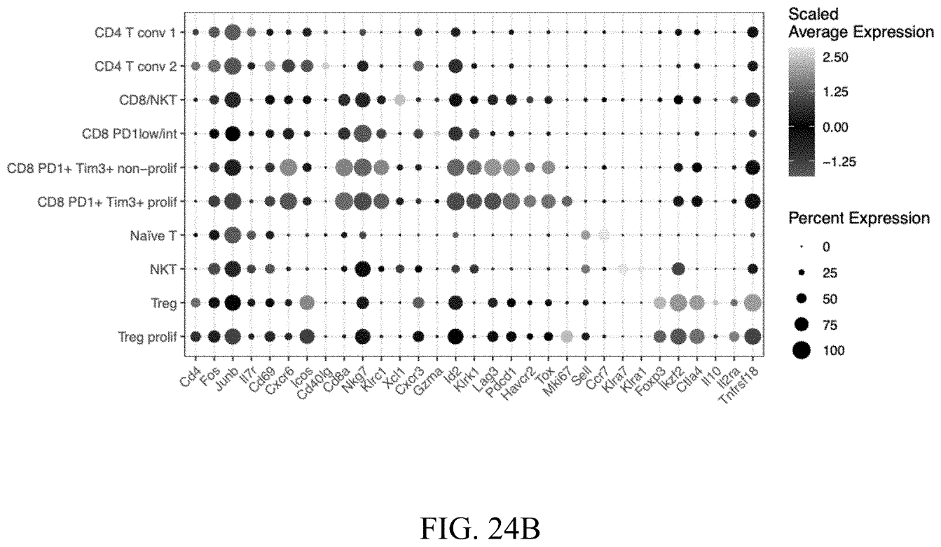

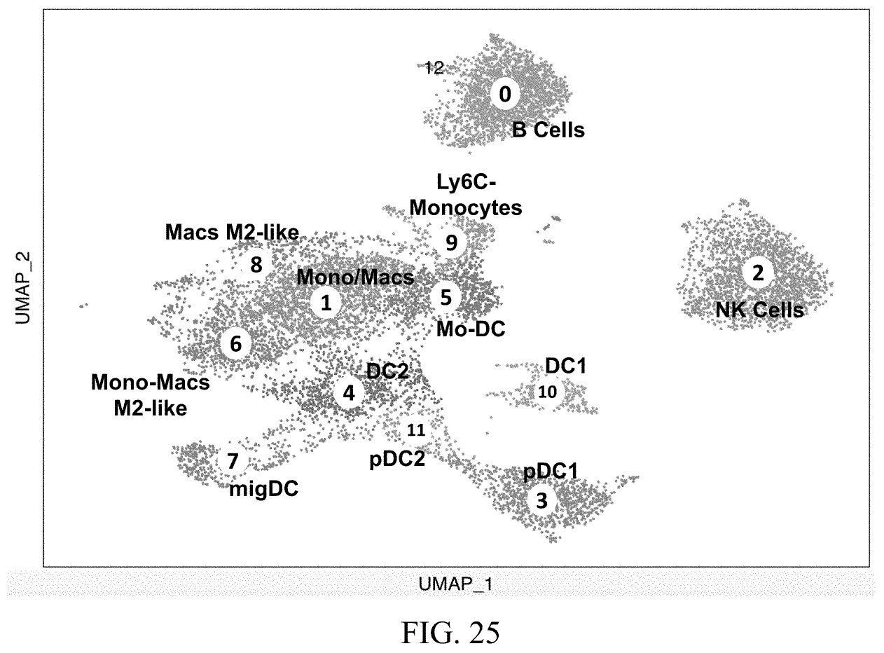

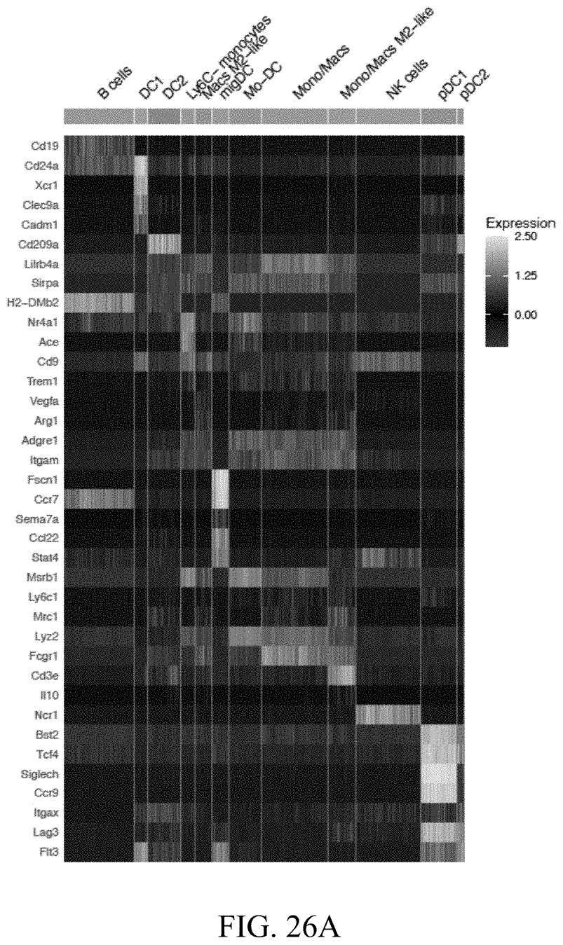

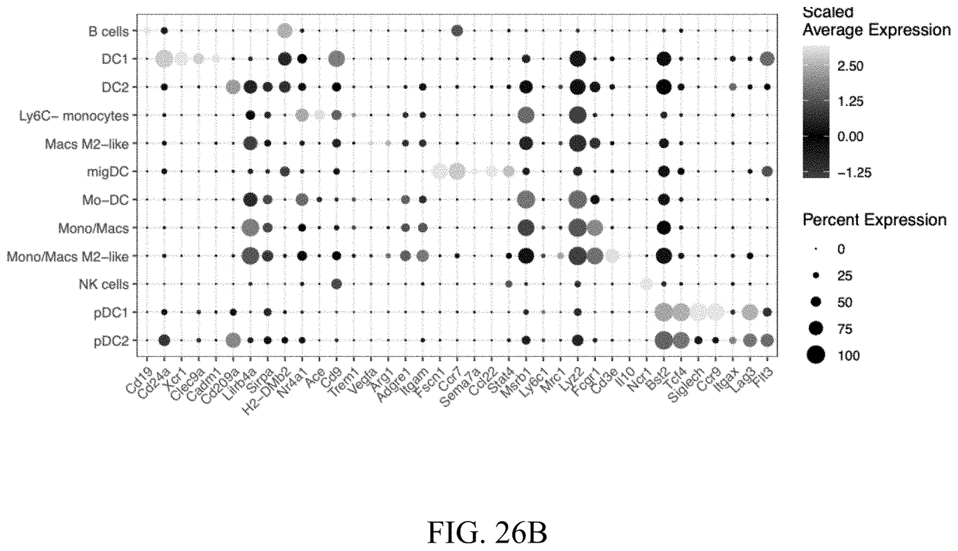

Applicants further refined the cell clusters to an analysis of T cell and non-T cell clusters and removed malignant cells from the analysis. The refined analysis was more extensive and annotates each cluster by cell type (FIGS. 23A and 25). UMAP analysis was performed on T cells and non-T cells to identify clusters of cells. The cells were annotated by cell type using cell type marker genes (FIG. 23-26).

### Example 2—Identification of Interacting Cells in the Tumor Microenvironment

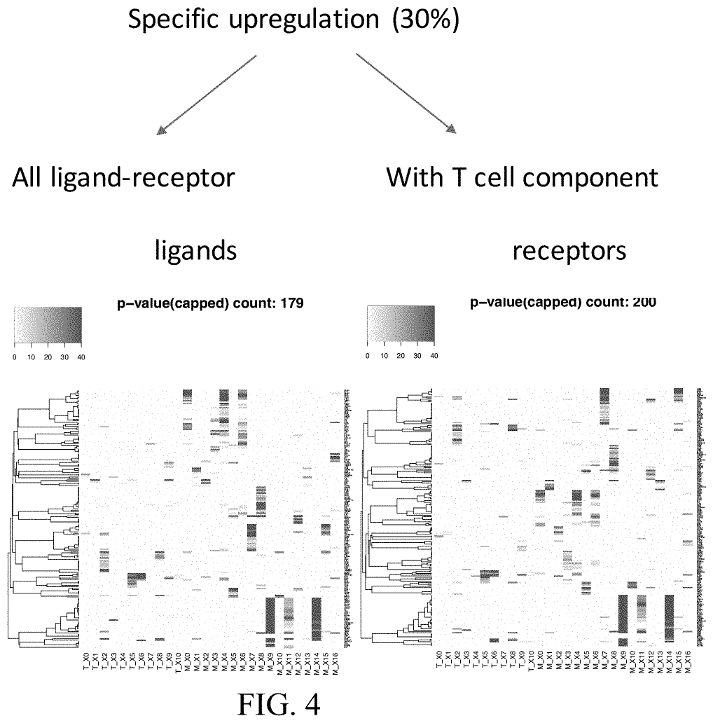

Applicants identified interactions for clusters based on ligand-receptor interactions under conditions where the ligands or receptors were specific or generally upregulated for the gene clusters (FIGS. 2, 3, 4, 5).

Applicants show that PDCD1 is expressed on CD8 T cells and its ligand PDCD1LG2 is expressed in clusters (FIG. 6A).

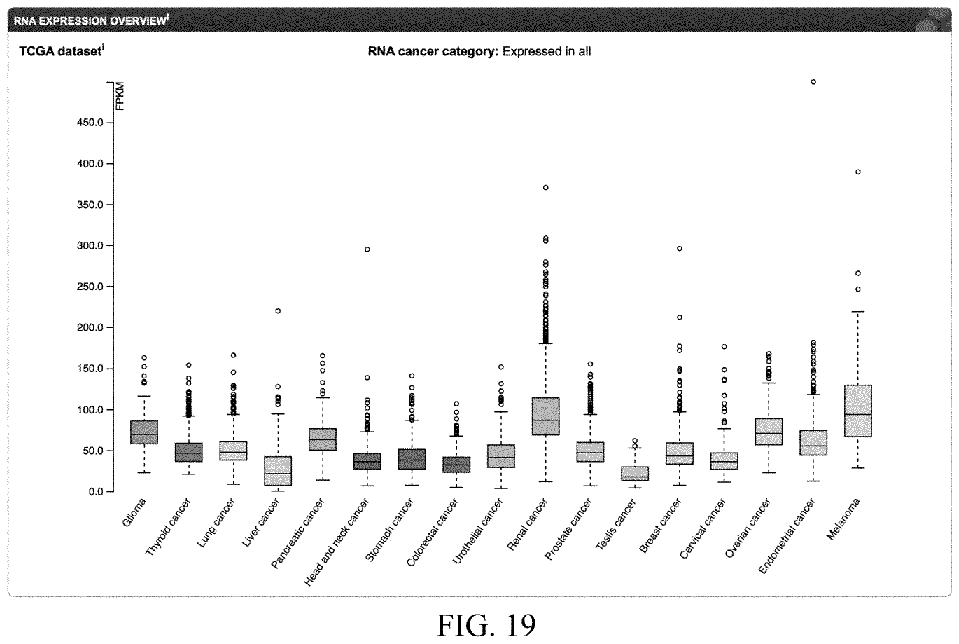

Applicants show that VEGFB is expressed exclusively by tumor cells and its ligand NRP1 is expressed on CD8 T cells (FIG. 6B). In endothelial cells, VEGF-B stimulation upregulated the expression of the fatty acid (FA) transport proteins (FATPs). In vivo, Hagberg et al. found that VEGF-B deficient mice had a reduced FA uptake, leading to significantly less FA accumulation in their hearts, muscles and brown adipose tissues (BATs) (Nature. 2010 Apr. 8; 464(7290):917-21). FA are preferred by memory CD8 rather than effector CD8 T cells, which tend to rely more on glycolysis. Also, iTregs rely on FA metabolism and Foxp3 is a driver of fatty acid oxidation enzymes expression. Thus, FA uptake by increased expression of VEGFB by tumors may increase Tregs in the tumor microenvironment. Yang et al., show in a human melanoma xenograft model that VEGFB remodels microvasculature rendering it leaky and promoting cancer cell extravasation. Importantly, VEGF-B expression correlates with poor prognosis (Proc Natl Acad Sci USA. 2015 Jun. 2; 112(22):E2900-9). Amongst the different human tumors (TCGA), VEGFB expression is highest in melanoma (FIG. 19).

Applicants show that CCR1 is expressed exclusively by macrophages and its ligand CCL3 is expressed on CD8 T cells (FIG. 6C).

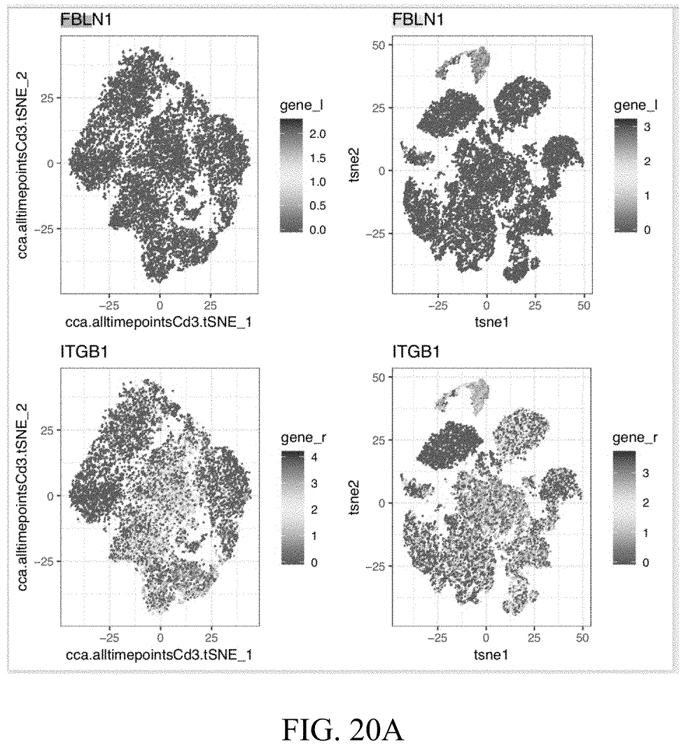

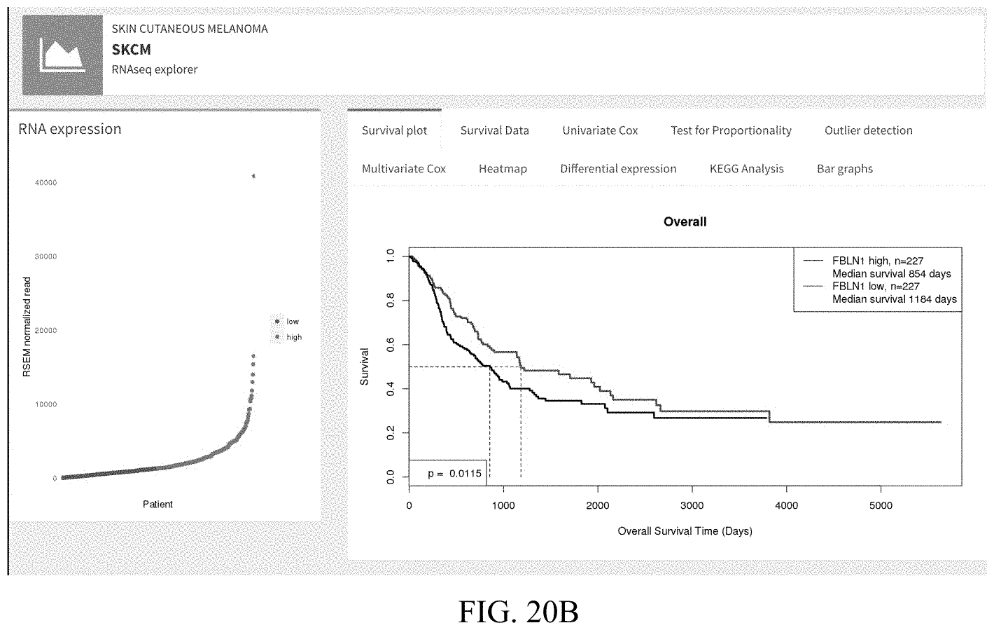

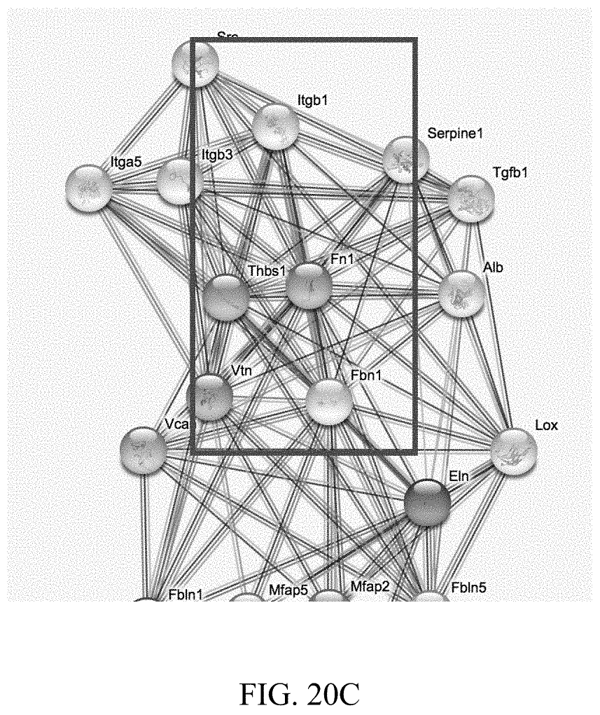

Applicants show that FBLN1 is expressed exclusively by tumor cells and its ligand ITGB1 is expressed on CD8 T cells (FIG. 20A). This interaction does not seem to be direct, but through FN1 (FIG. 20C). FBLN1 expression along with FN1 has been implicated in breast cancer Doxorubicin resistance. In bladder cancer and colorectal cancer, FBLN1 seems to correlate with a good prognosis. The role of FBLN1 in cancer is not clear. FBLN1 may play a role in TGFβ activation along with FN1.

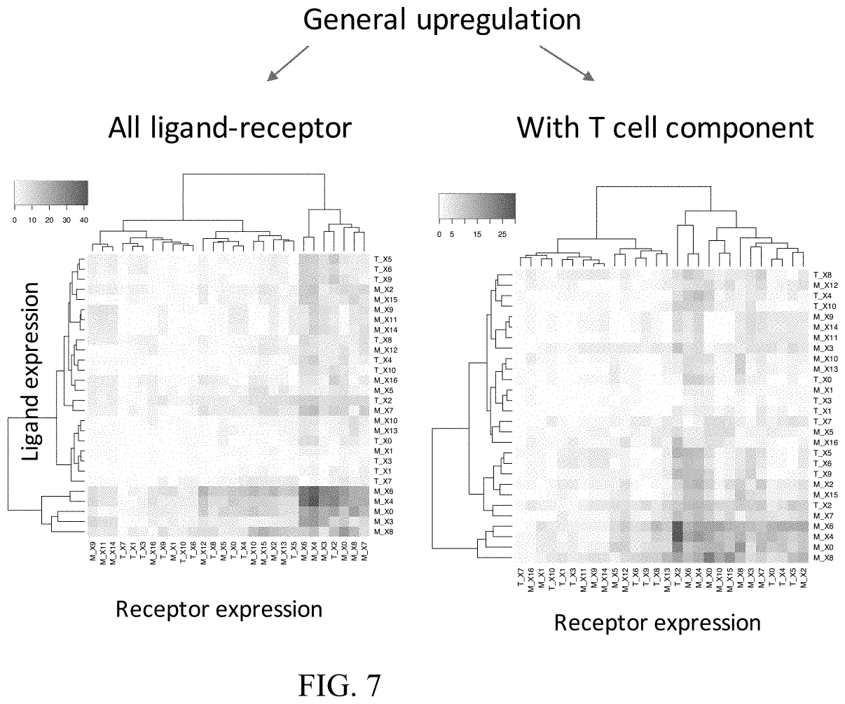

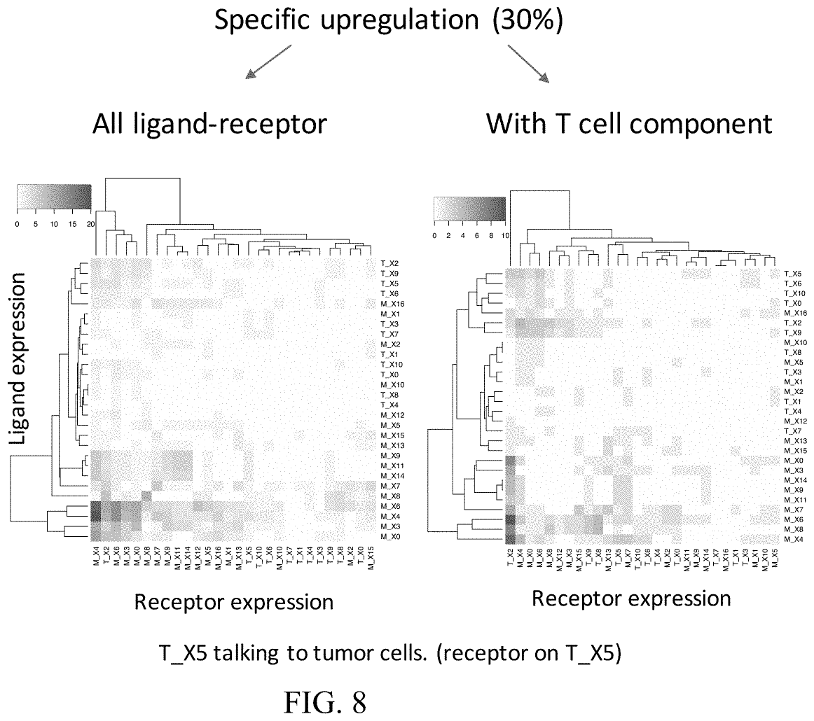

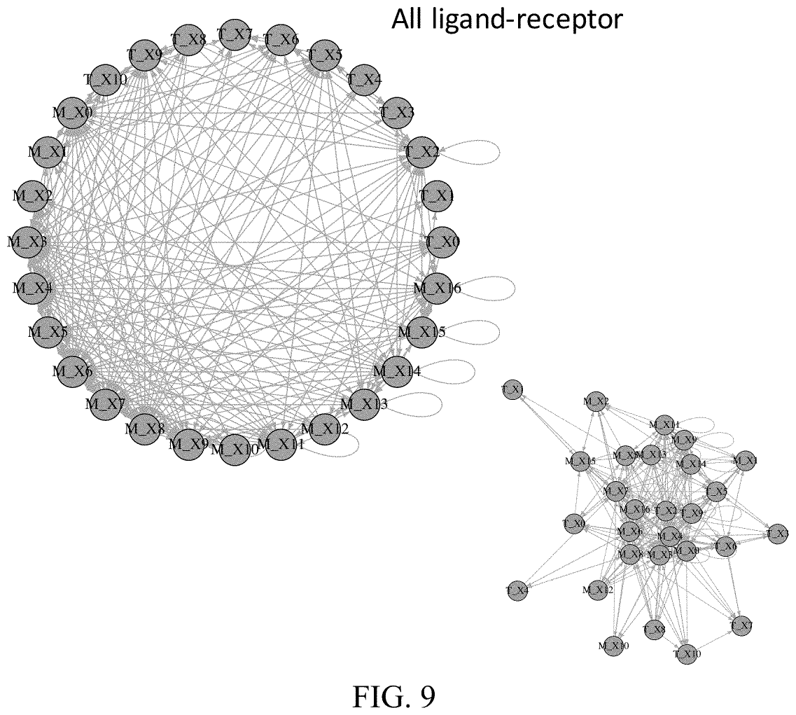

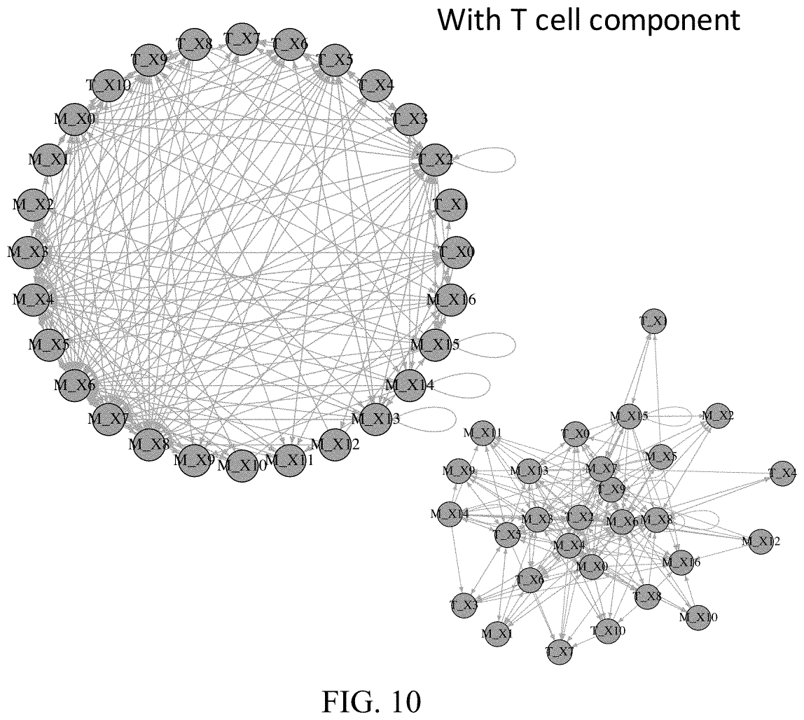

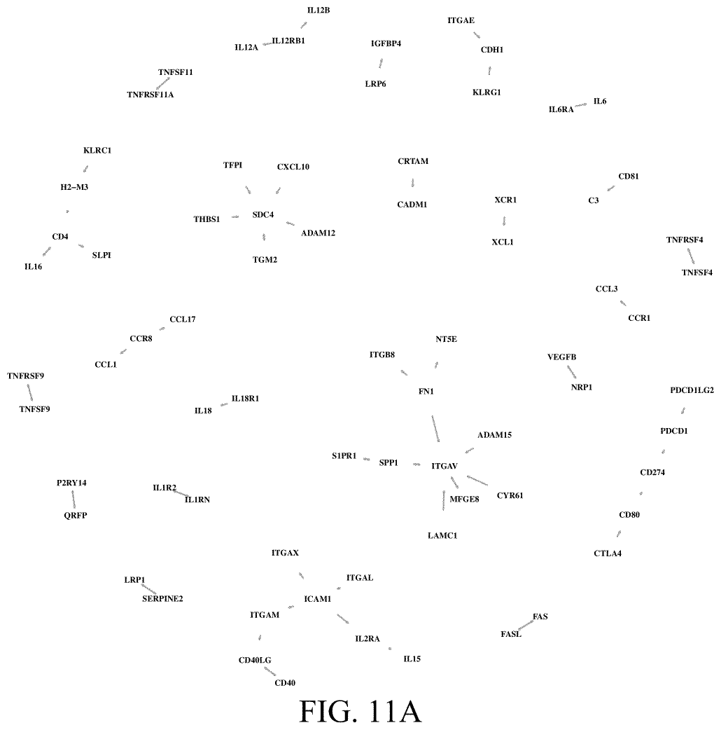

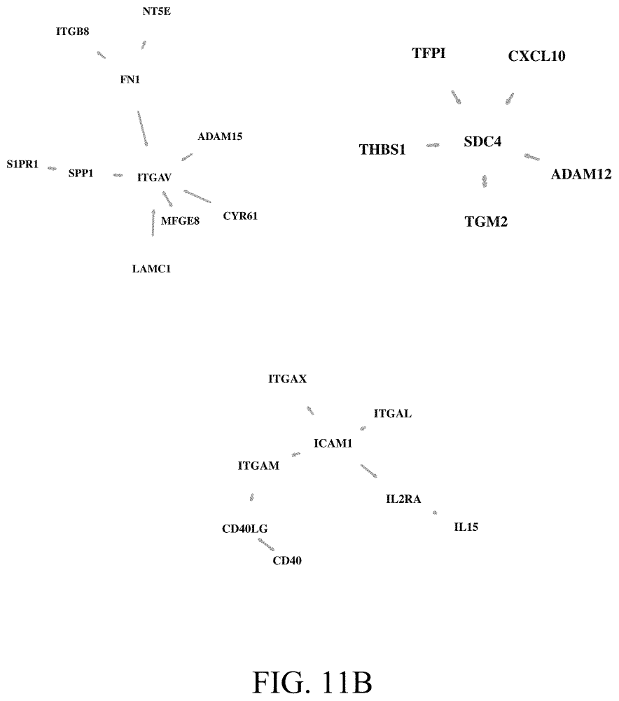

Applicants identified specific and generally interacting clusters (FIGS. 7, 8). Applicants generated network interaction maps for all of the identified clusters (FIGS. 9, 10). Applicants identified ligand receptor networks and specific genes that are at the hubs of the networks (FIG. 11A,B).

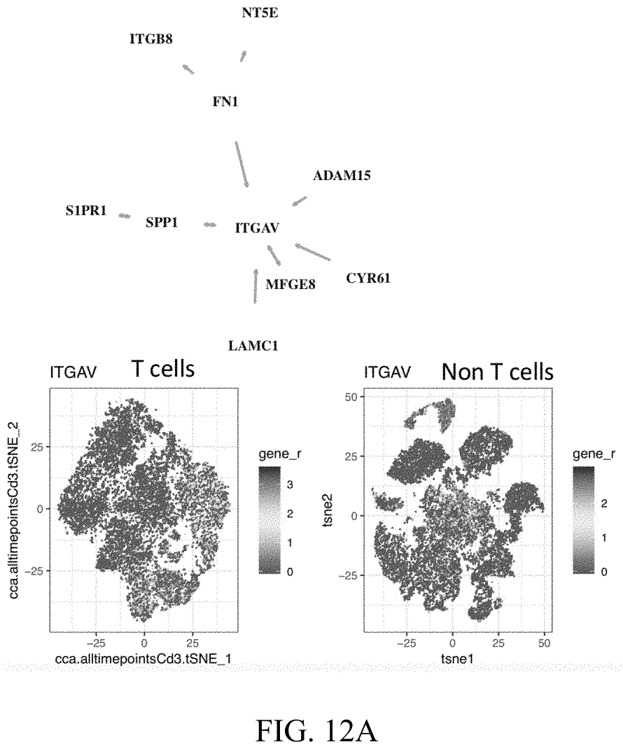

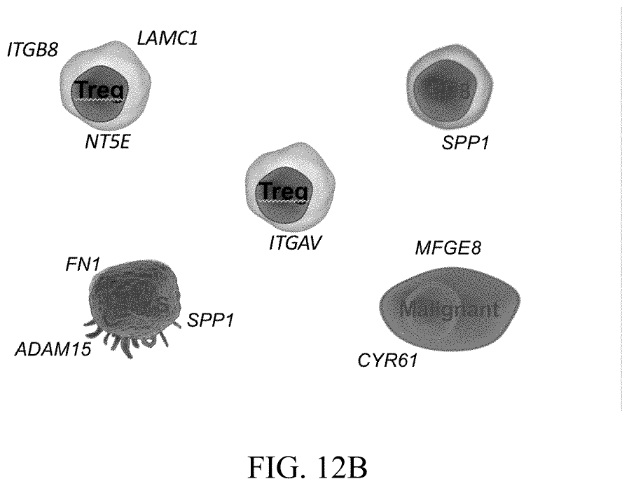

Integrin Alpha V (ITGAV) was found at one hub (FIG. 12). ITGAV is mainly expressed by Tregs. It encodes for integrin αV β1 subunit which heterodimerizes with different β chains (αVβ1, αVβ3, αVβ5, αVβ6 and αVβ8). This integrin subfamily recognizes ligands containing the Arg-Gly-Asp (RGD) tripeptide motif. Extracellular matrix (ECM) components, through specific peptide motifs such as Arg-Gly-Asp (RGD), interact with integrins and can modify the behavior of cells. Transforming growth factor-beta1 (TGF-beta1) is the main cytokine involved in the synthesis of ECM proteins.

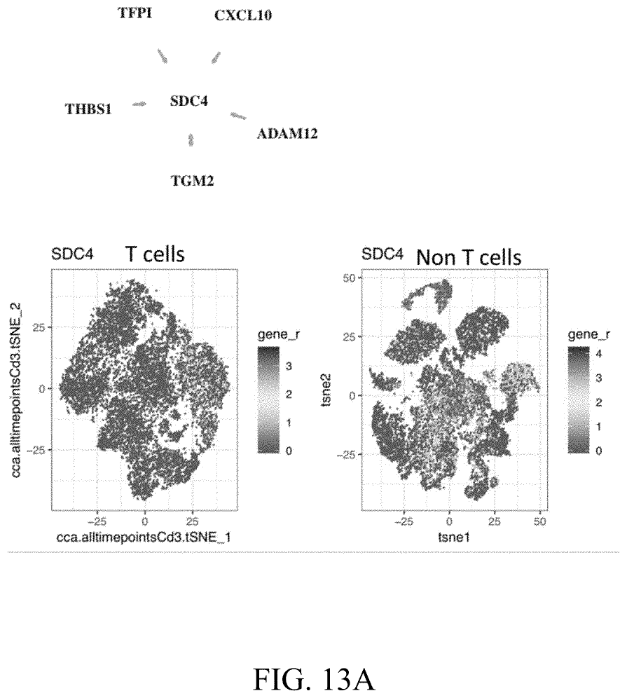

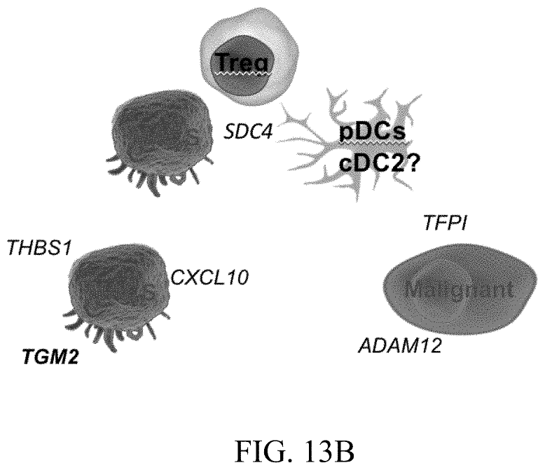

SDC4 was found at one hub (FIG. 13). Syndecan 4 (SDC4) is a transmembrane (type I) heparin sulfate proteoglycan. SDC4 can act as a receptor and co-receptor in mediating intracellular signaling. SDC4 is associated with adhesion, membrane trafficking and has a role in MAPK and Mtor pathways.

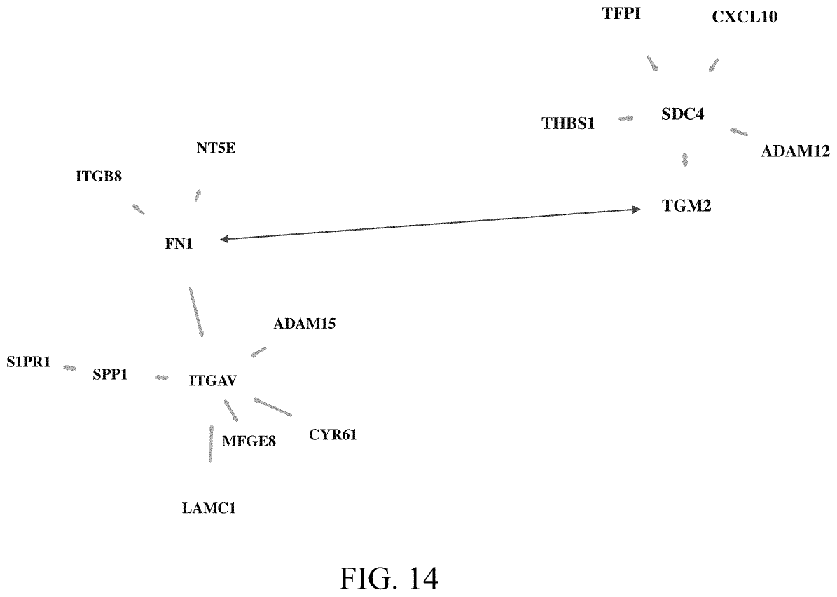

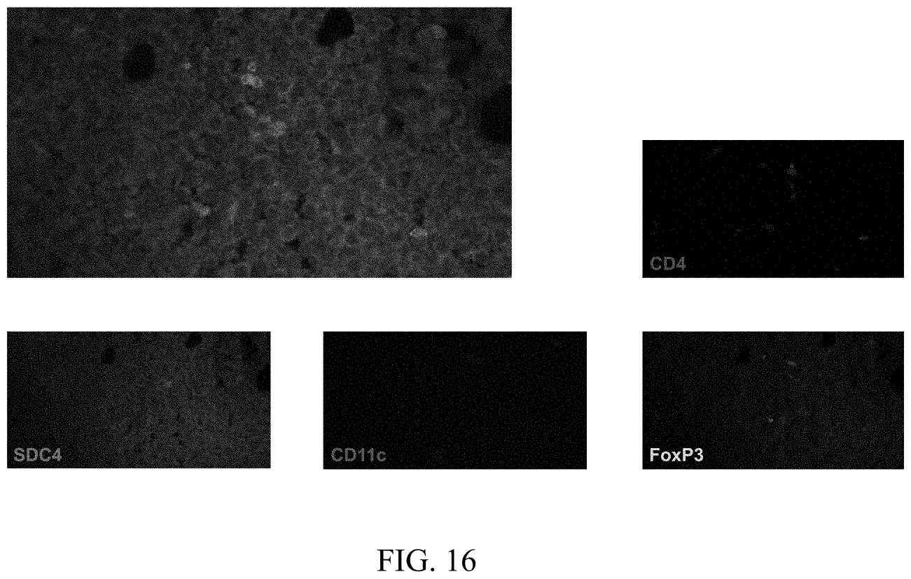

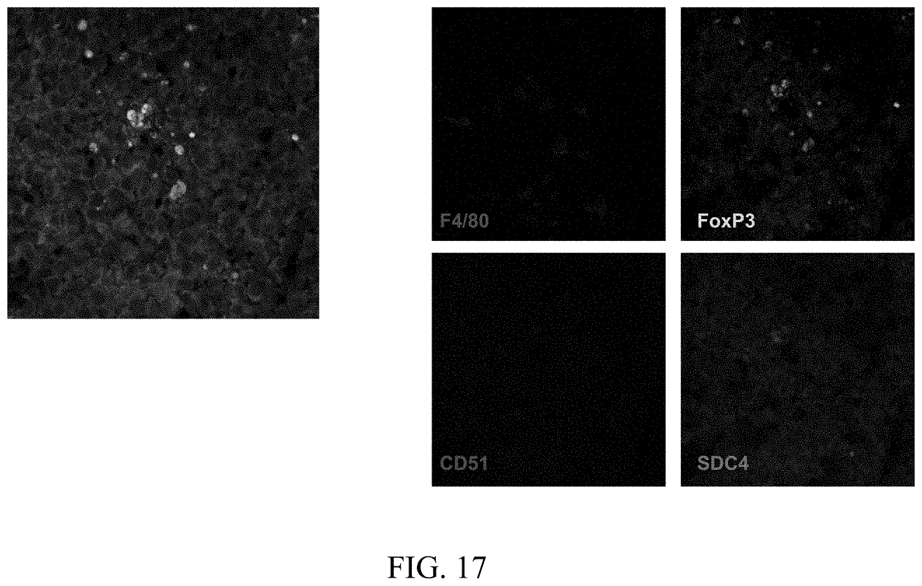

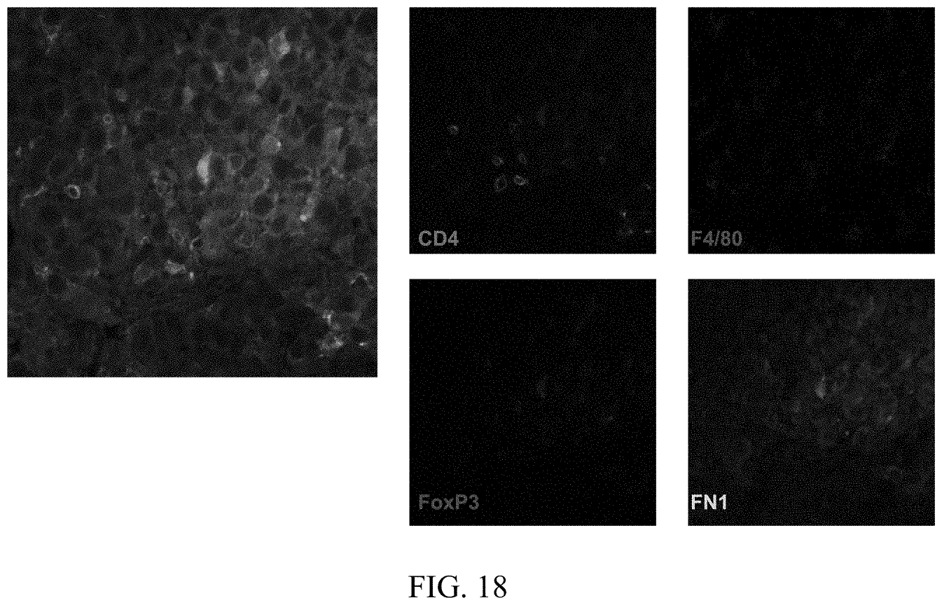

Applicants also identified that the ITGAV network interacts with the SDC4 network through FN1 and TGM2 (FIG. 14). Applicants performed immunohistochemistry to validate the interactions between cells expressing SDC4 (FIG. 16, 17), and Tregs expressing FN1 (ITGAV hub) (FIG. 18).

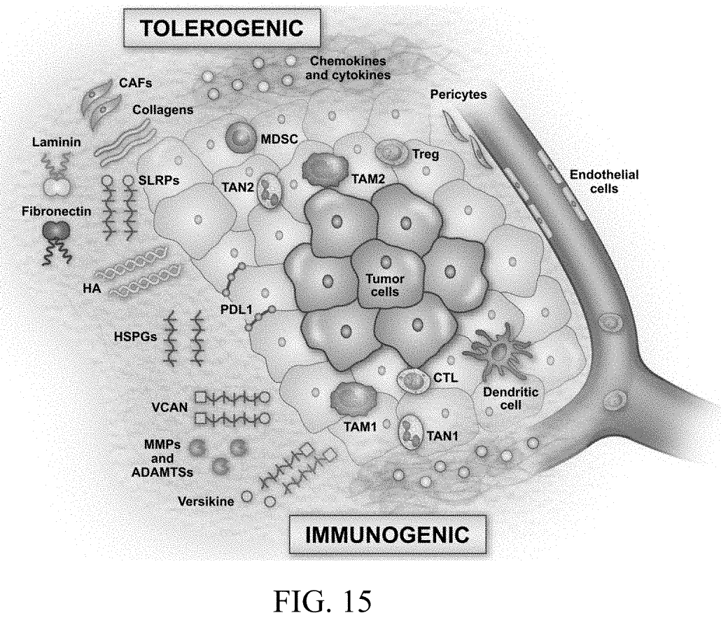

Applicants identified immune cells that interact directly with tumor cells. The tumor cells may suppress effector T cells and activate Tregs through interactions with the immune cells. Applicants identified interactions between immune cells in the tumor microenvironment. Modulation of these interactions may also enhance an anti-tumor response. For example, immune cells may communicate with one another to promote the wound healing function, such as, through ITGAV-FN1-ITGBA-TGM2-SDC4 interactions. The interactions can be blocked in vivo to treat cancer. Applicants can generate effector T cells for adoptive cell transfer (CAR T, TILs) that are modified, such that they do not interact with the tumor cells. In conclusion, the TME is an intricate milieu of cells hosting the tumor, including infiltrating myeloid and lymphoid cells, stromal and mesenchymal cells, and ECM components. Matrix remodeling shapes the inflamed immune microenvironment. Tumor-infiltrating Tregs and regulatory myeloid cells, including MDSCs, TAMs and TANs, promote a tolerogenic TME (FIG. 15). The ligand-receptor network data provide herein suggests that Treg cells represent a central hub interacting with myeloid cells and activated CD8 T cells, while orchestrating ECM remodeling.

Applicants used the cell cluster annotations (FIGS. 23A and 25) and single cell RNA expression data to identify up-regulated ligand-receptor pairs in the clusters and to characterize interactions amongst cell types. Table 1 shows all of the ligand receptor pairs and the cell types expressing the ligand and receptor.

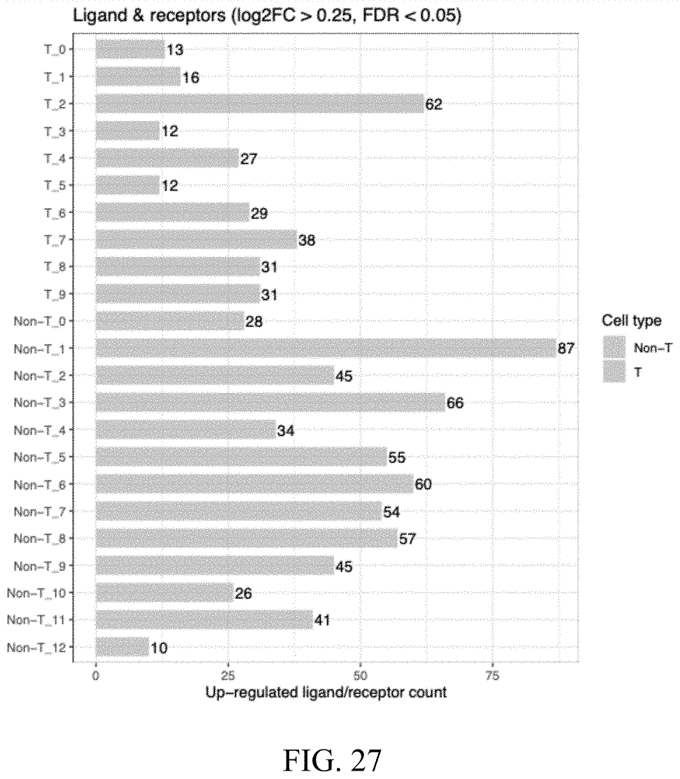

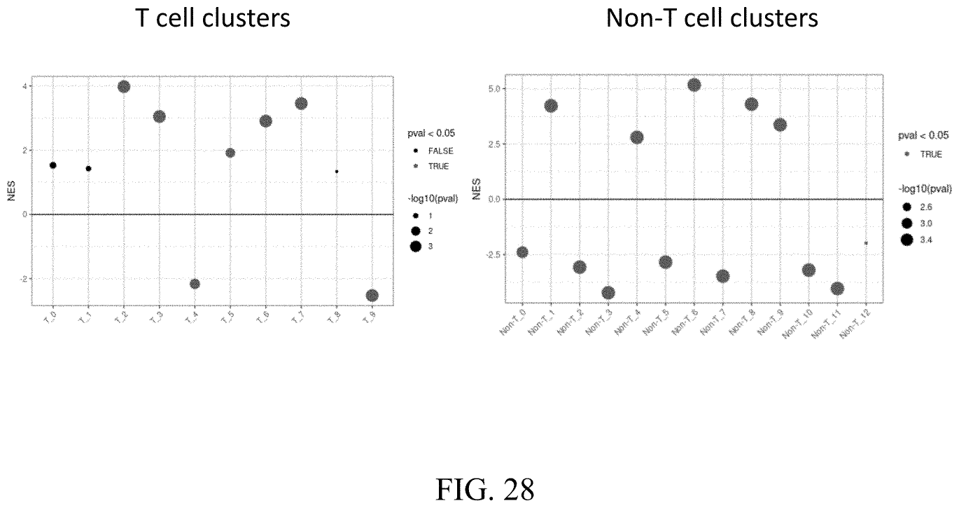

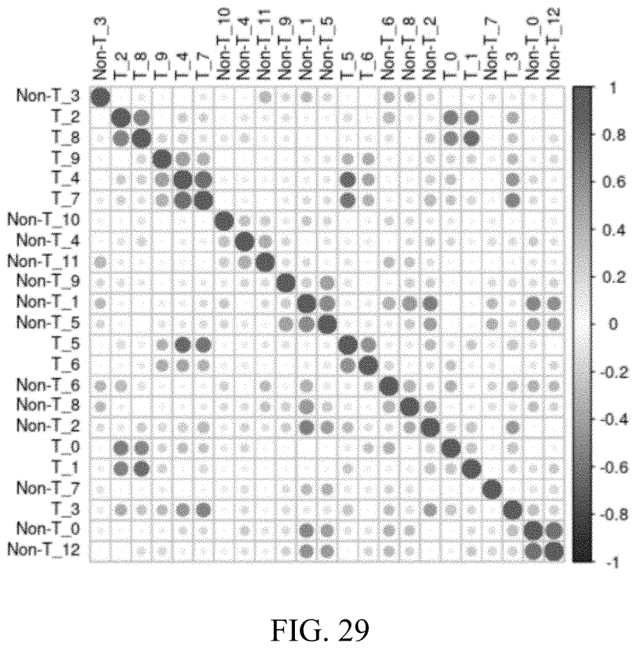

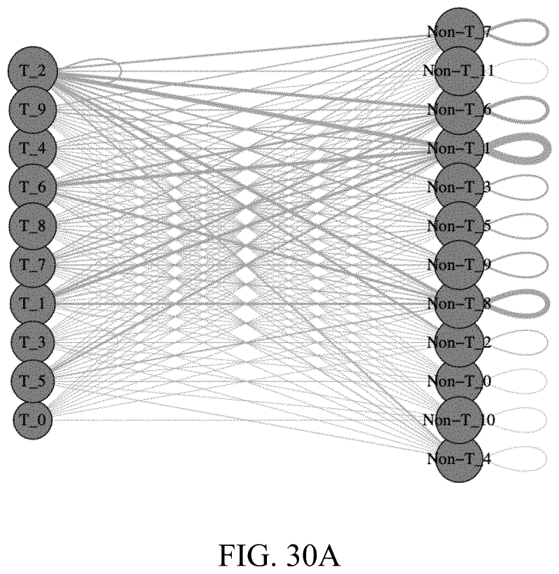

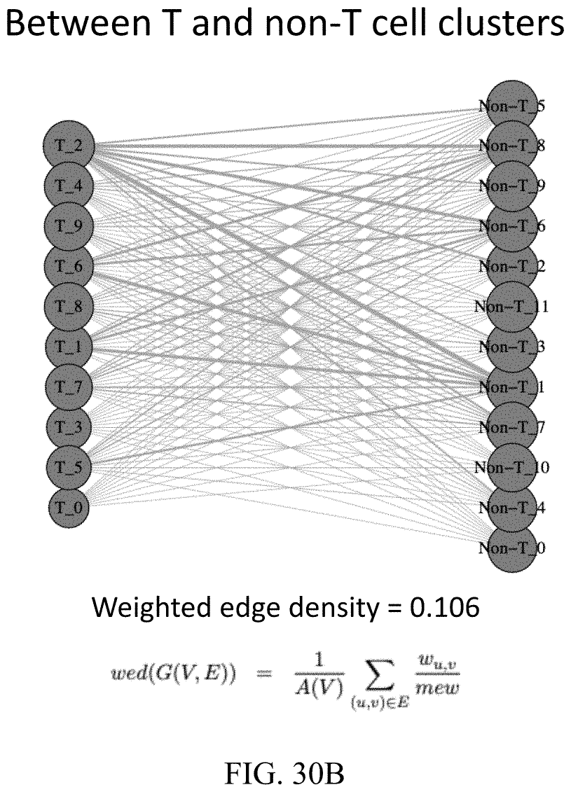

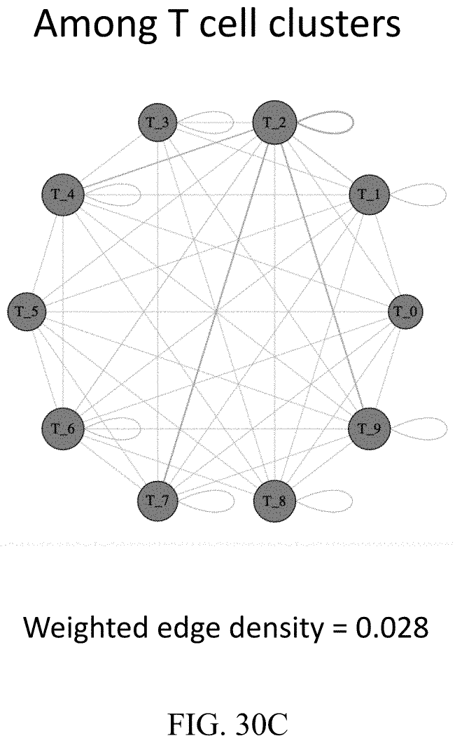

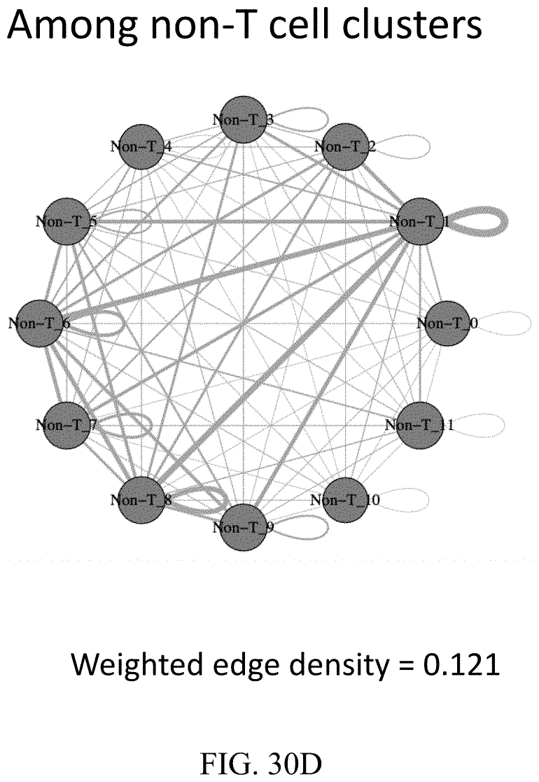

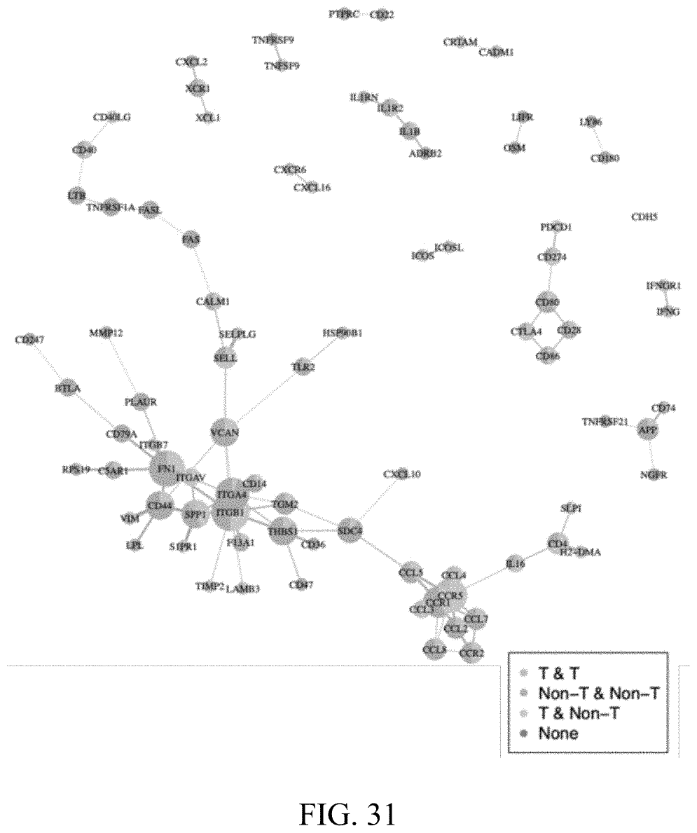

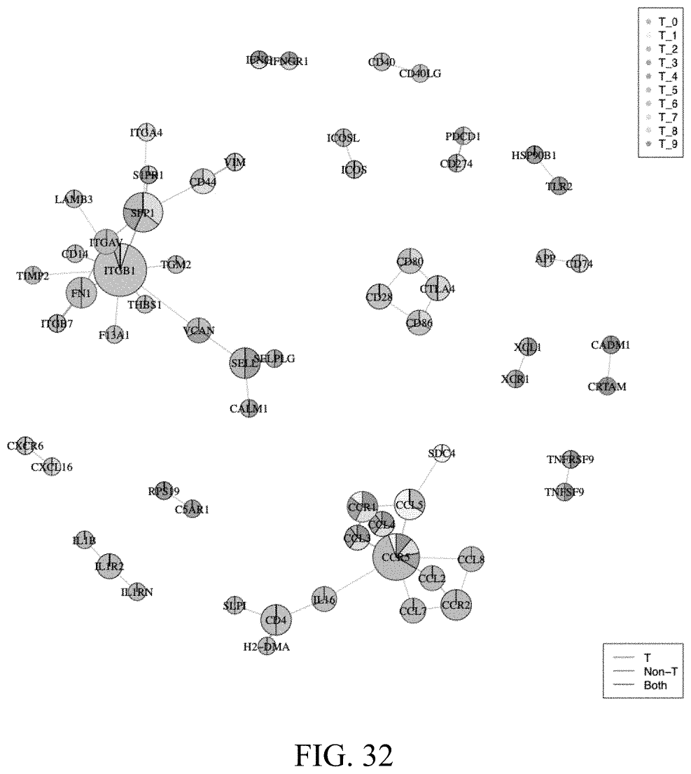

Applicants determined the cells having the most potential interactions by quantitating the number of upregulated ligands or receptors for each cluster (FIG. 27). Gene Set Enrichment Analysis (GSEA) was used to identify statistically enriched ligand/receptor upregulation. The normalized enrichment score (NES) indicates clusters enriched in upregulated genes (above the line) (FIG. 28). Applicants analyzed cluster similarity (FIG. 29). Applicants generated network maps of interacting clusters (FIG. 30). The network edge equals an interaction and the width of the edge is the number of interactions. The wedge density is highest between T and non-T cells. Tregs have the highest interactions between cells. Applicants determined gene networks for interactions between ligand receptor pairs that are upregulated in the clusters (FIG. 31) and further determined gene networks for interactions between T and non-T cell clusters (FIG. 32).

### Example 3—Identification of Tumor Microenvironment Dynamics During Tumor Progression

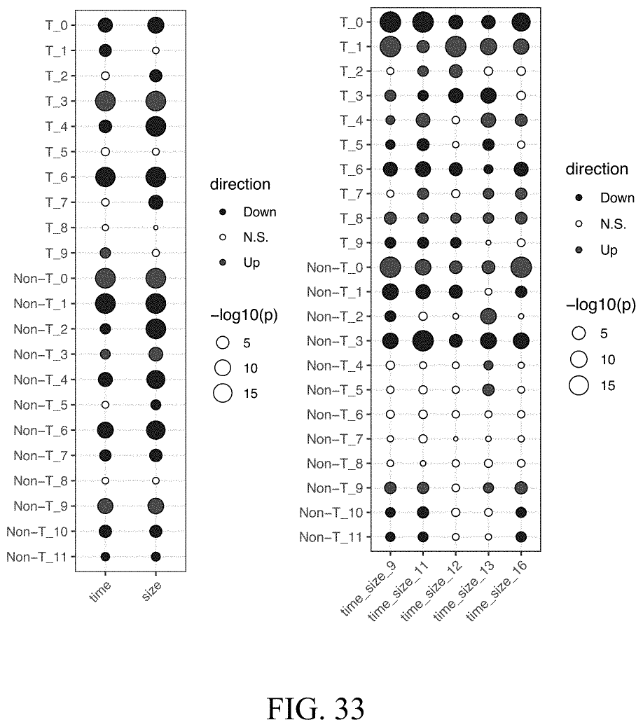

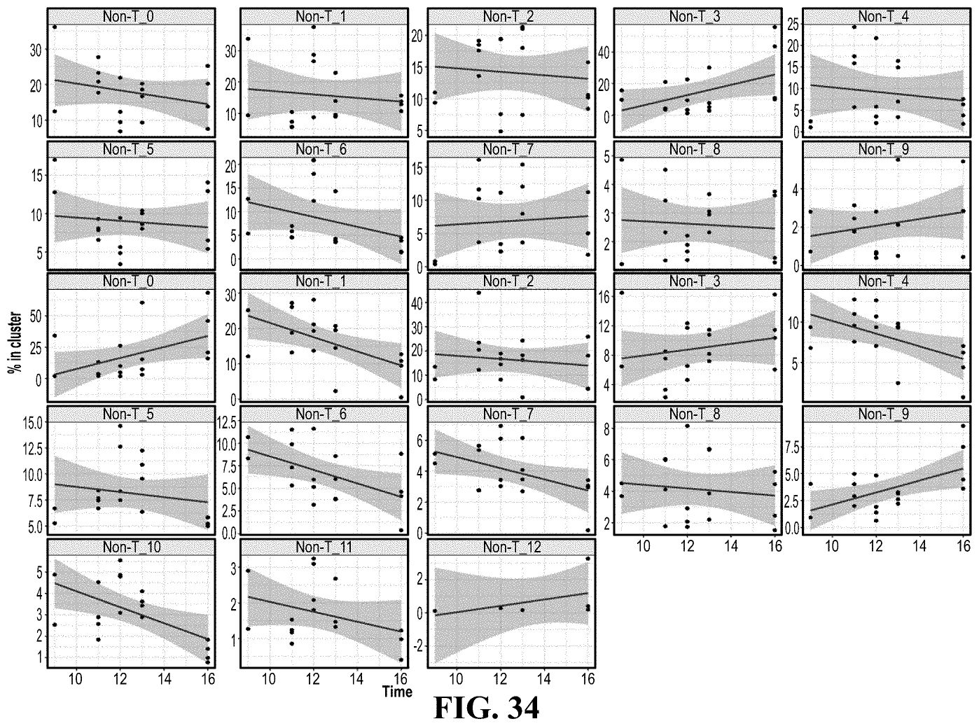

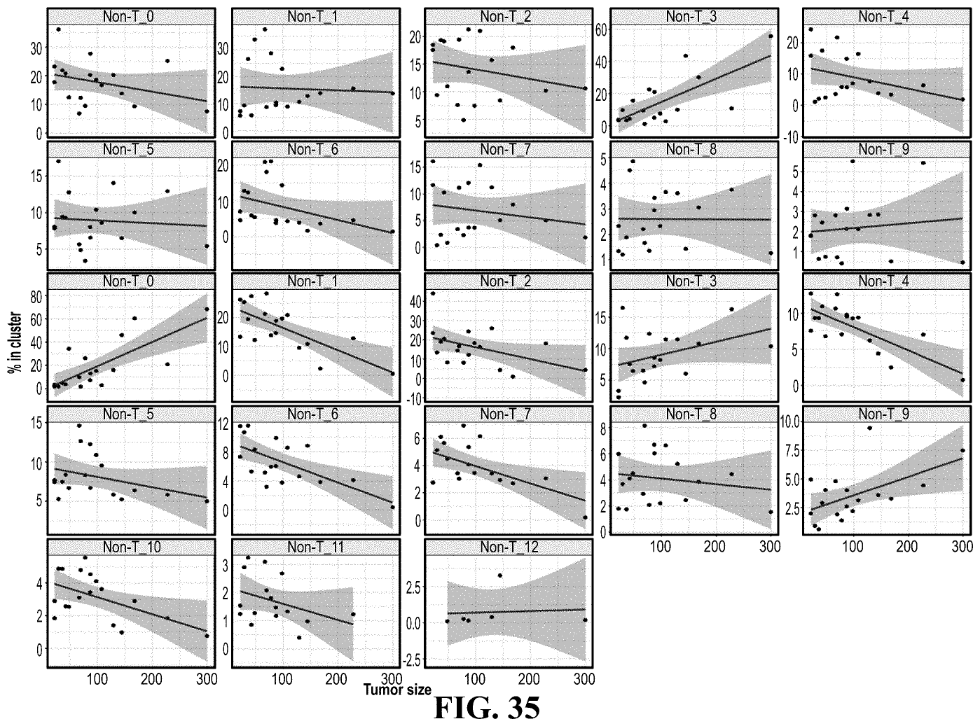

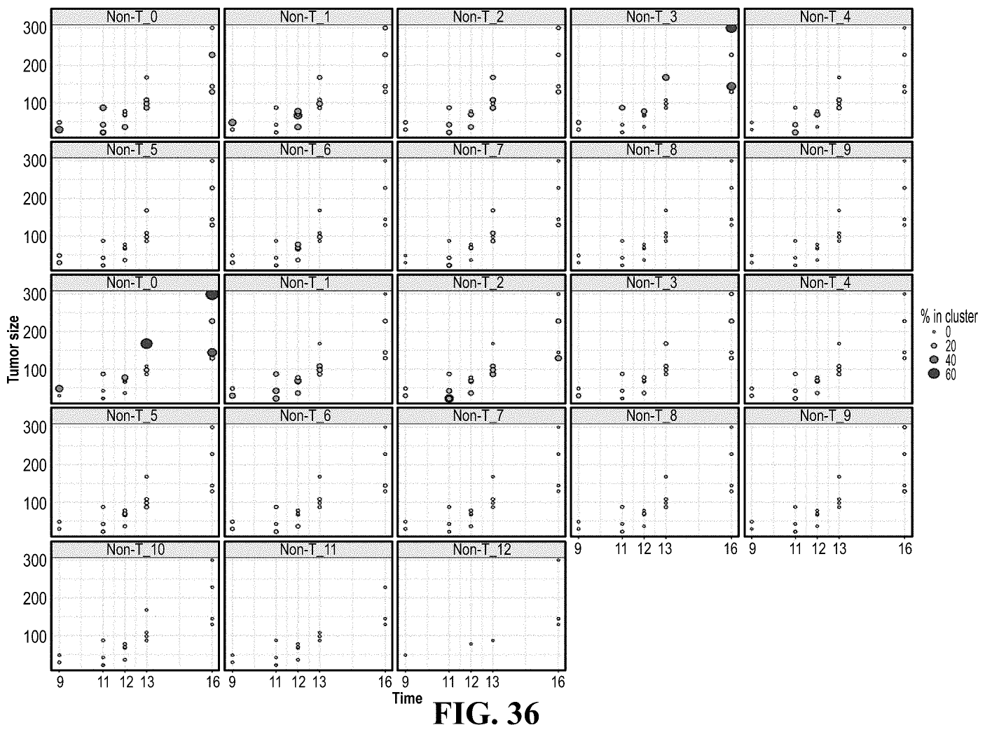

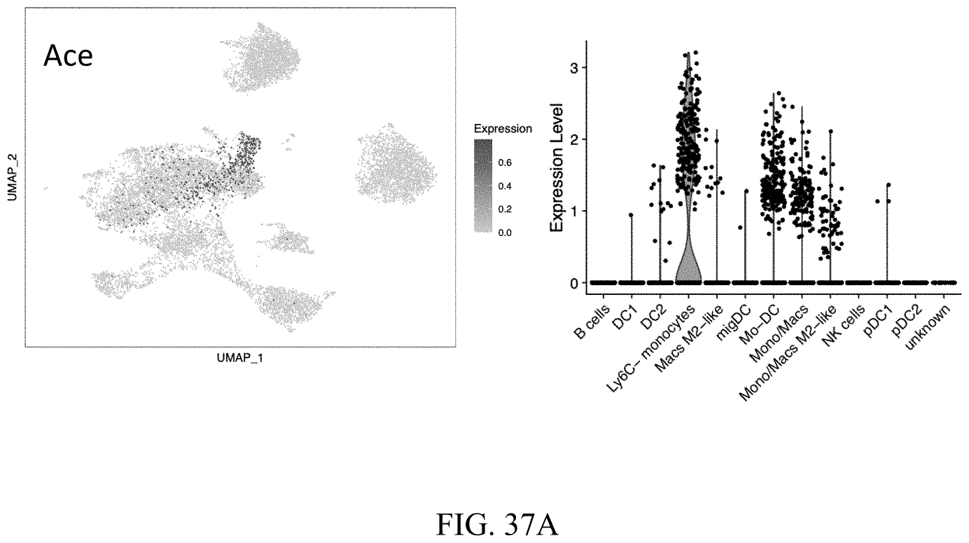

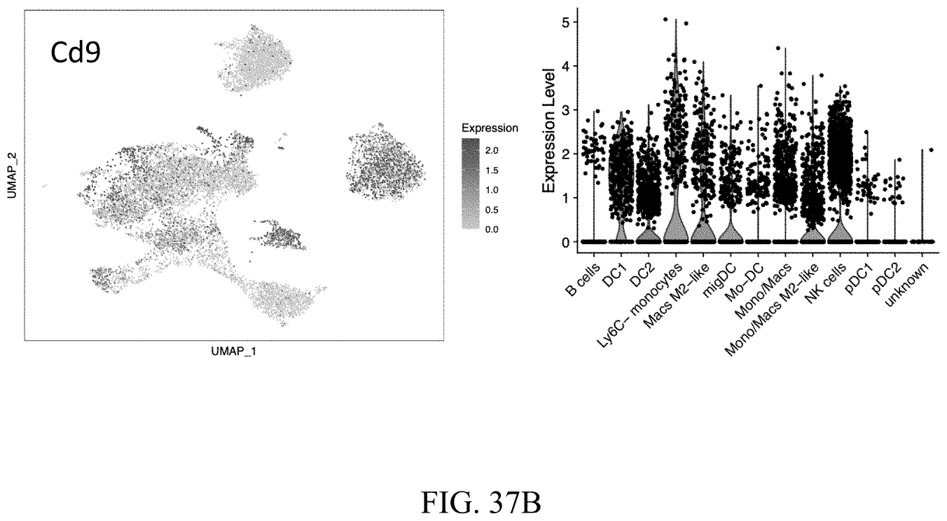

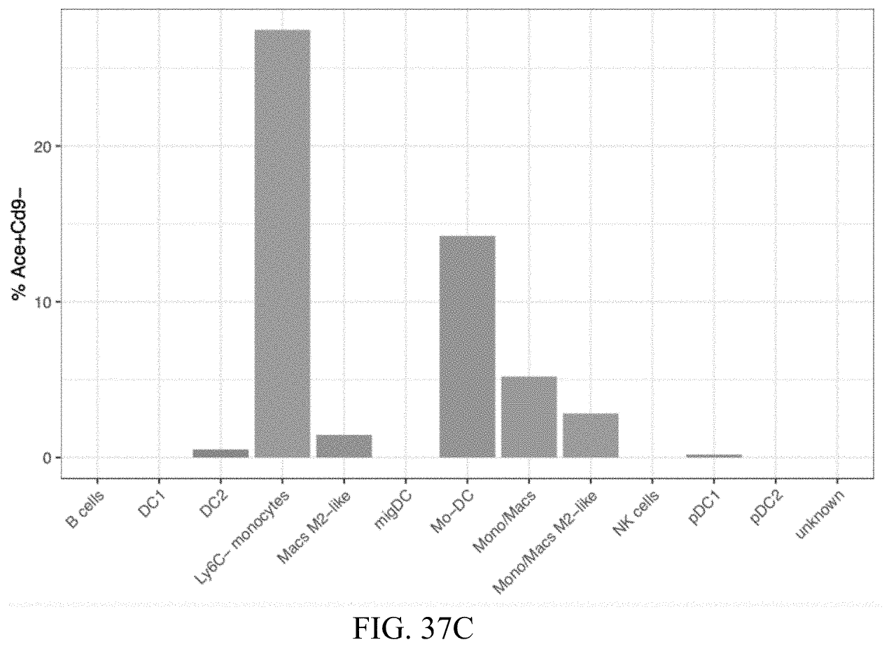

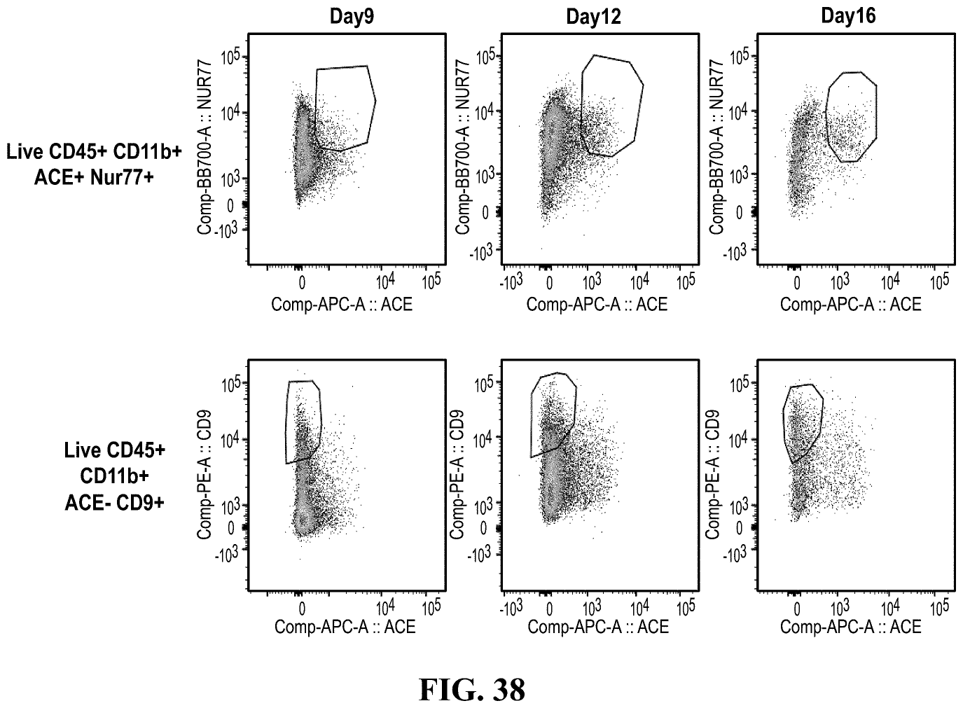

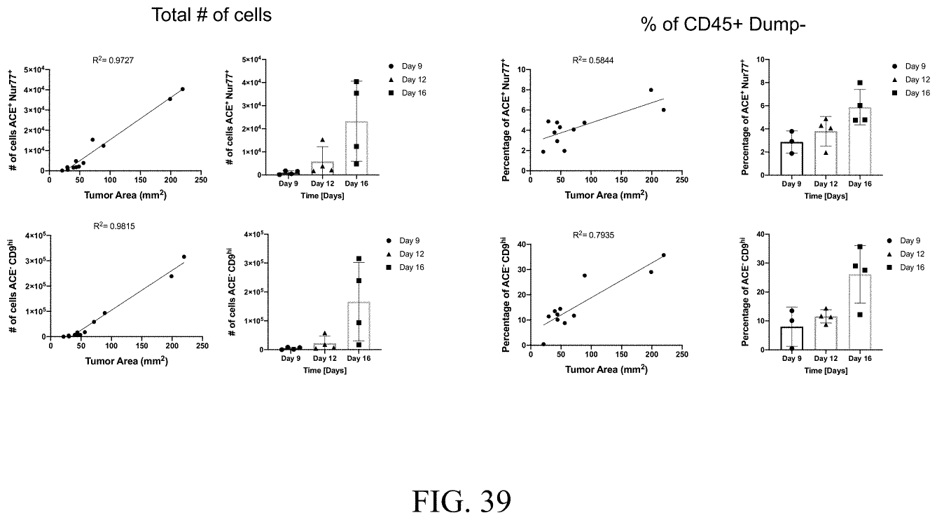

Applicants used the time course study with the B16 melanoma tumor mouse model to examine the change in frequencies of different cell types (clusters) (FIGS. 23A and 25) as a function of time after tumor implant and size of tumor. Applicants show that the clusters are associated with time and size of the tumor and are either positively or negatively associated (FIG. 33-36). Applicants validated the annotation of Ly6C-monocytes using a positive marker (ACE) and a negative marker (CD9) (FIG. 37-39).

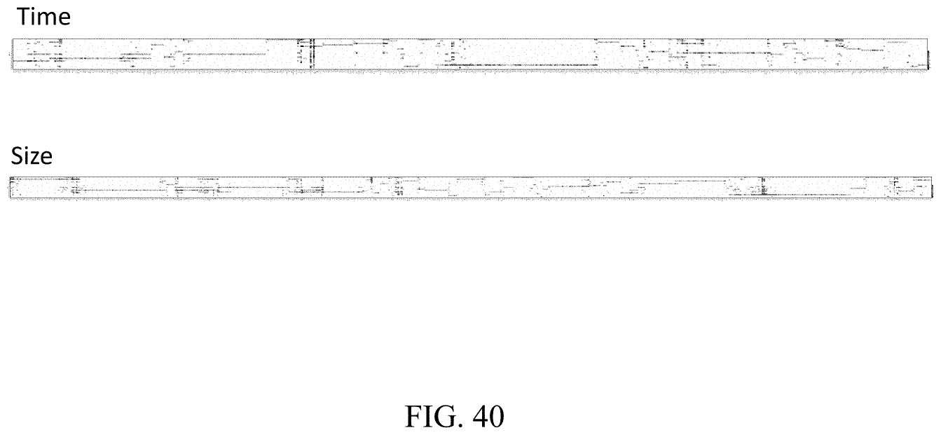

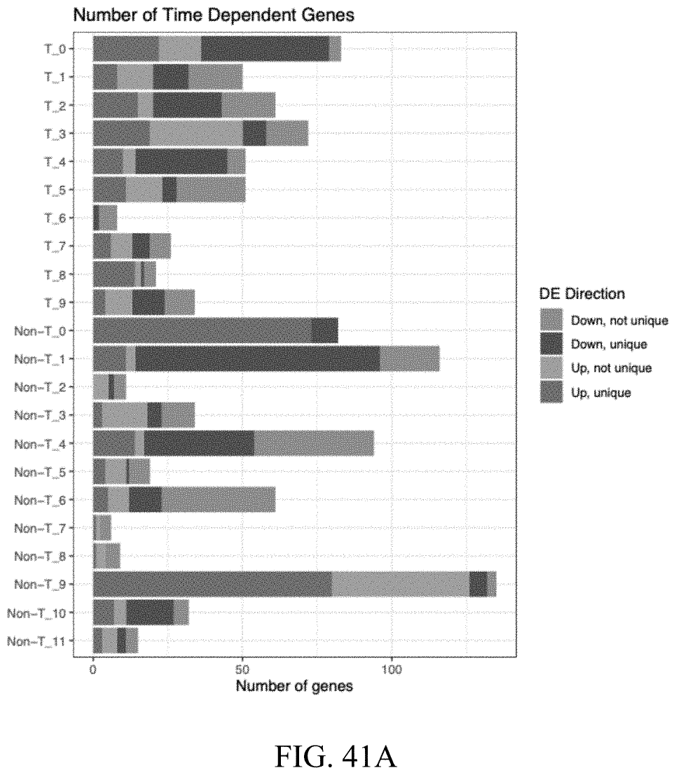

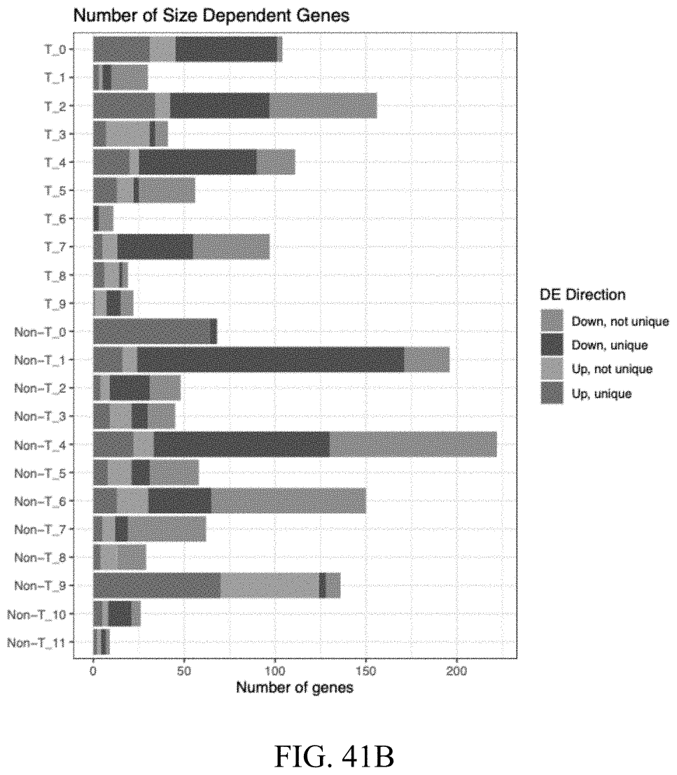

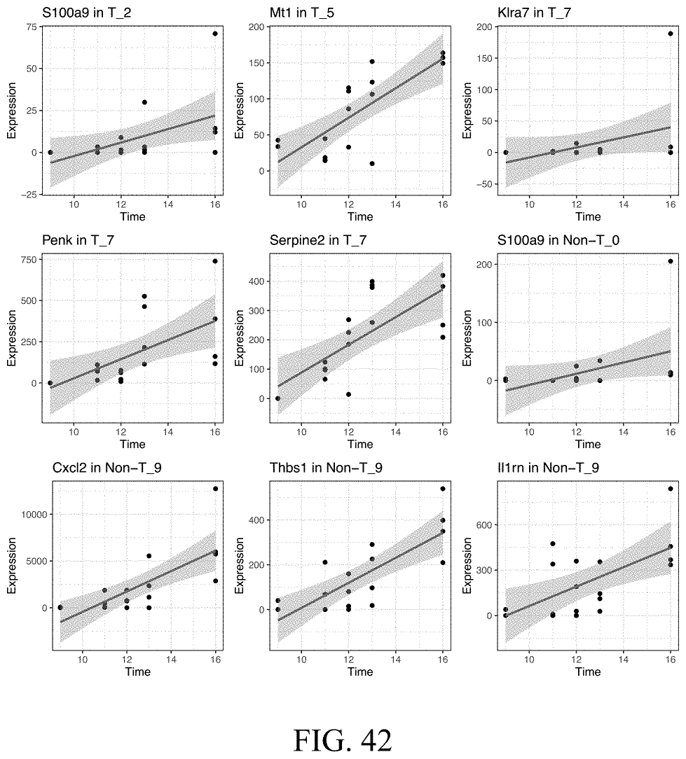

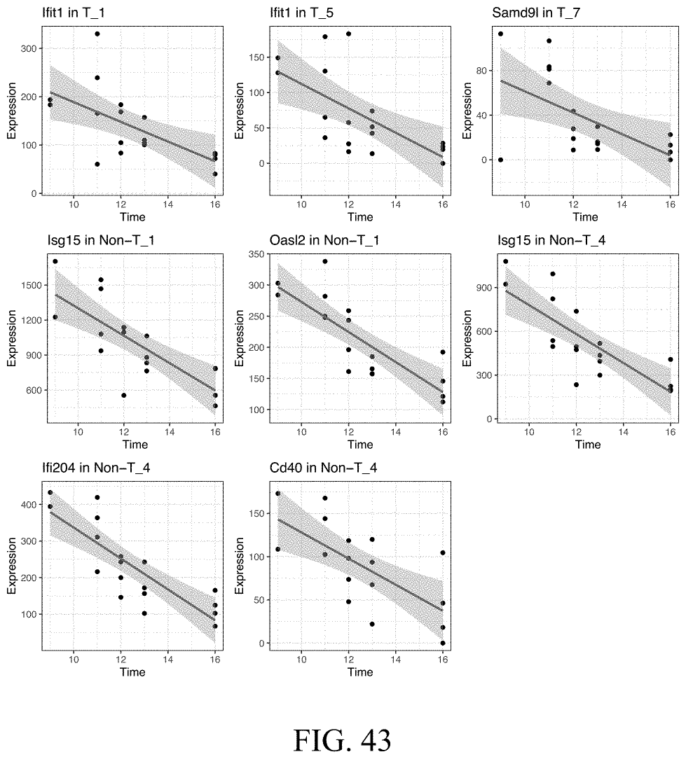

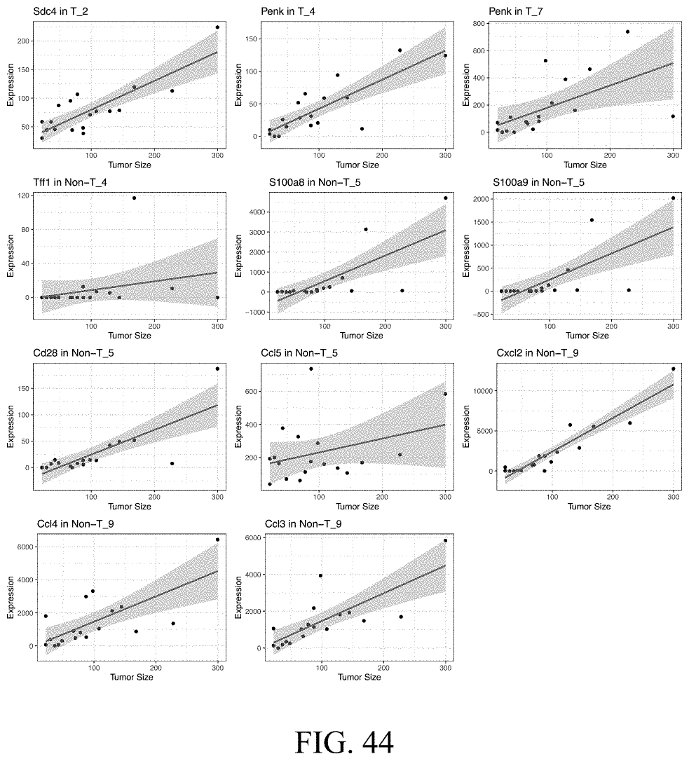

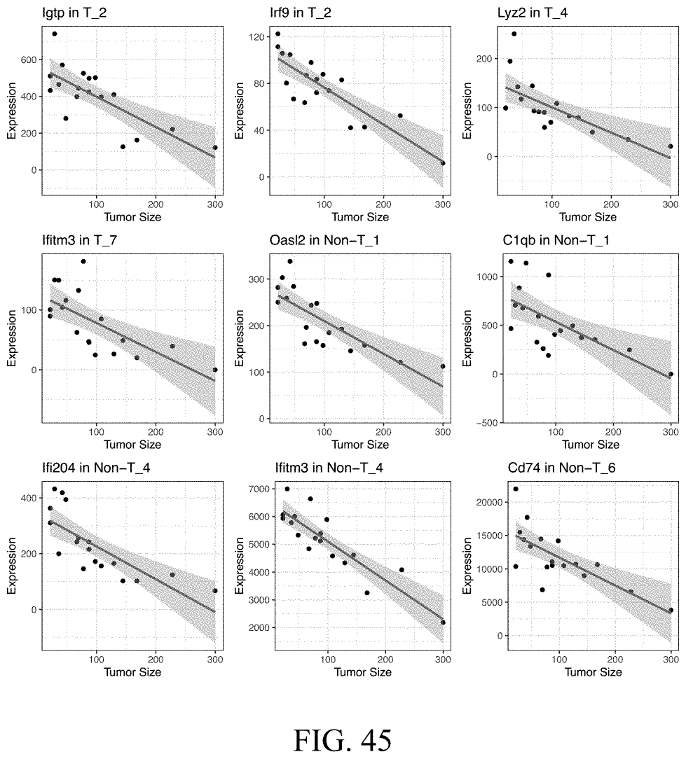

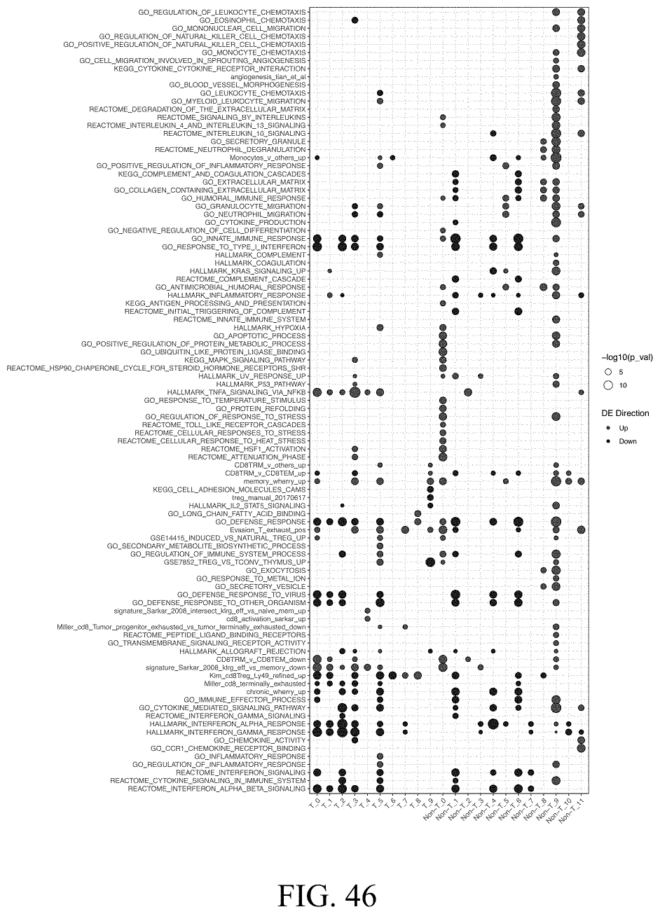

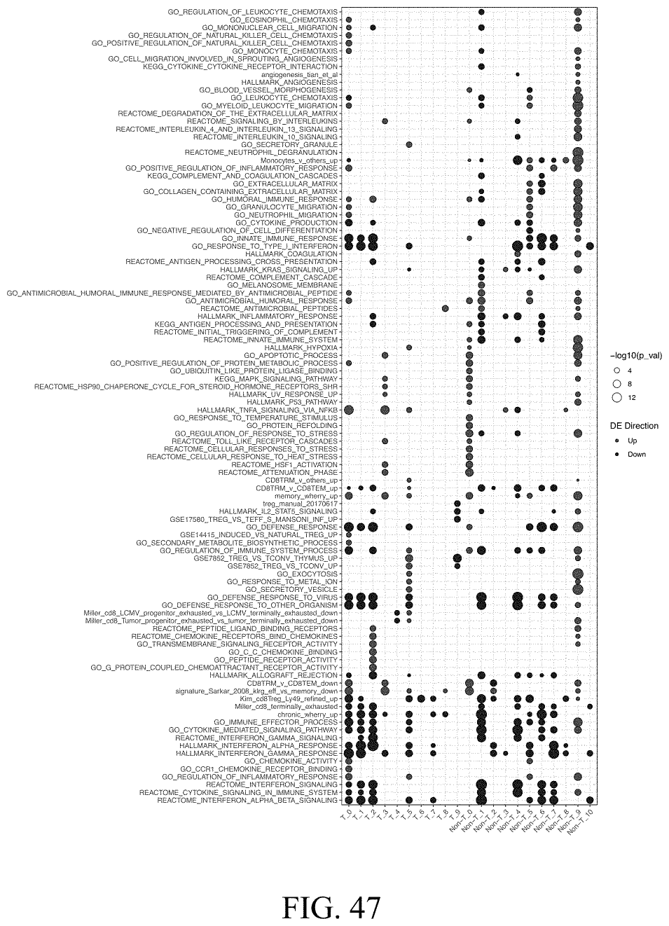

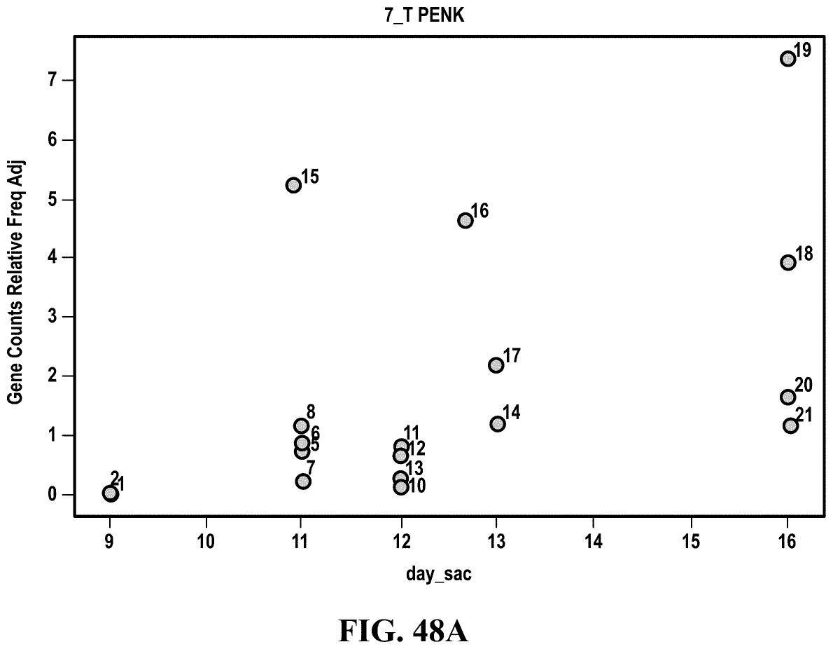

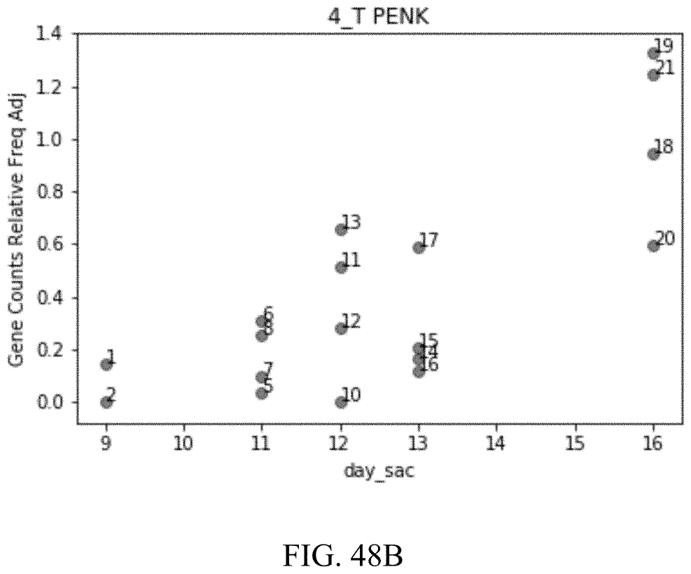

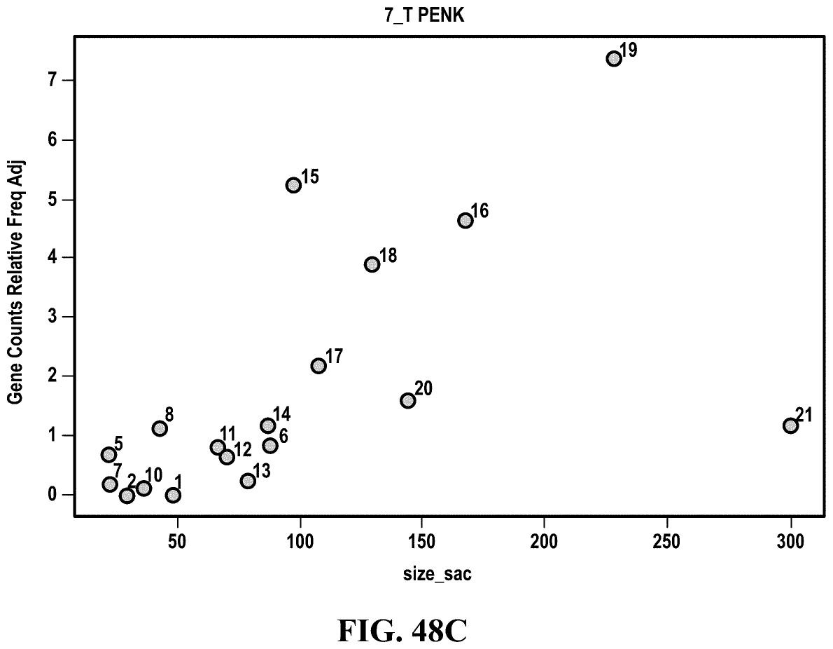

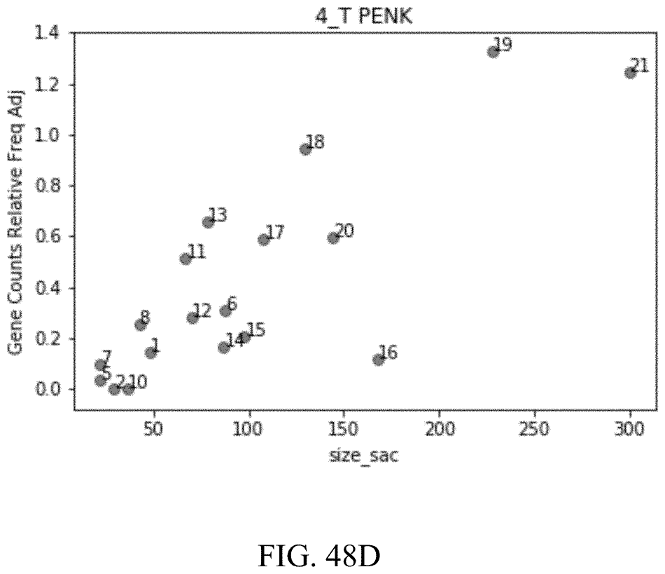

Applicants also analyzed changes in cluster specific gene expression as a function of time after tumor implant and size of tumor. Applicants used the time course study with the B16 melanoma tumor mouse model to identify genes that are differentially expressed over time and tumor size for each cell type (FIGS. 23A and 25 clusters) (Table 2 and 3). FIG. 40 visualizes genes that are positively and negatively correlated with time and tumor size. Every row depicts a cluster and every column is a gene. The figures show genes that are cluster specific as they are correlated horizontally across the figures or genes that are tumor specific because they are correlated vertically in the figures (i.e., correlated in every cluster). Applicants identified genes positively and negatively associated with time and size and that are unique to each cluster (FIG. 41). Example time dependent and tumor size dependent genes that are positively and negatively associated are shown (FIG. 42-45). GSEA pathways were determined for time dependent and size dependent genes (FIG. 46-47). Applicants further validated time and size dependent expression of PENK in cluster T_4 and T 7 (FIG. 48). These clusters are both CD8+PD1+ TIM3+ T cell clusters. PD1+ TIM3+ cells have been shown to be the most exhausted cells. The receptor for PENK is opioid growth factor receptor (OGFr), which is ubiquitously expressed among the immune cells in the tumor microenvironment.

### Example 4—Opioid Receptor Signaling in Tumor-Infiltrating CD8 T Cells

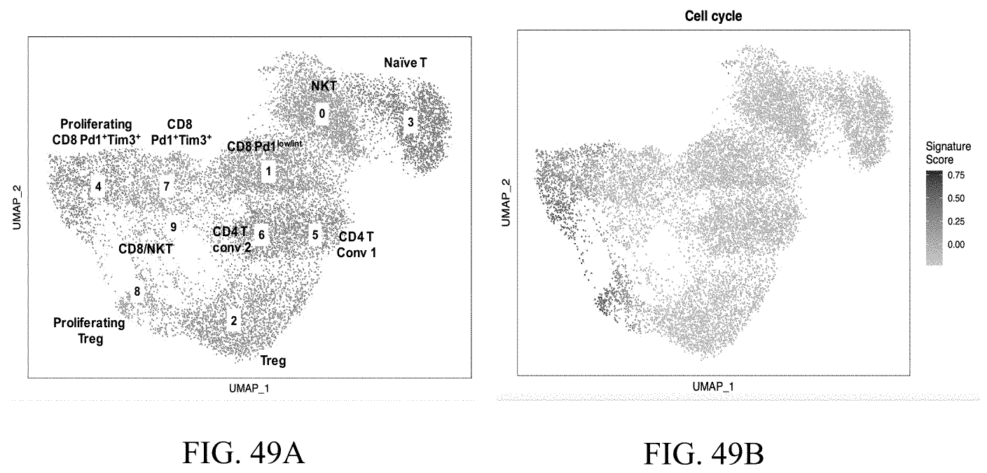

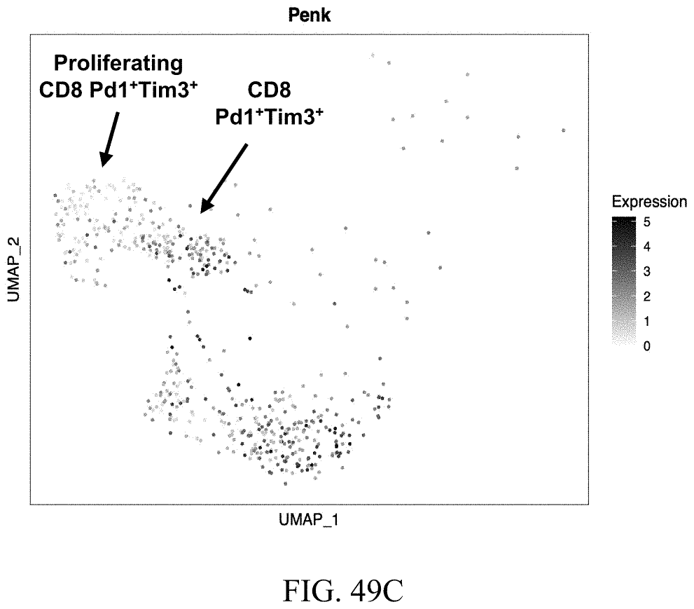

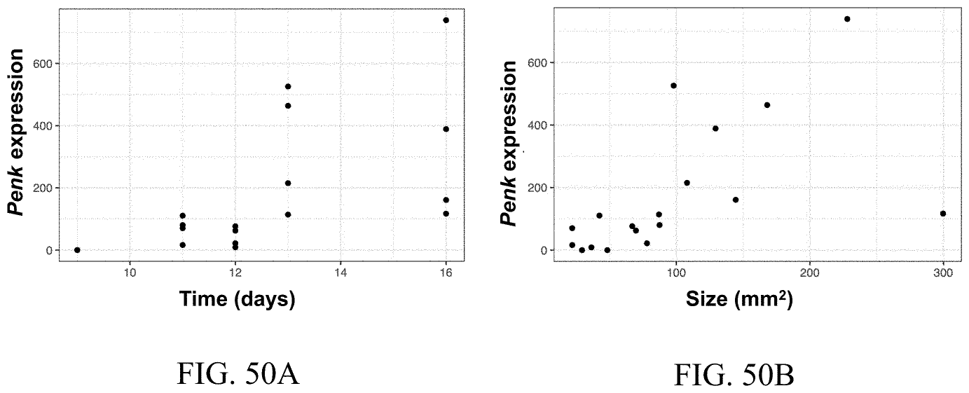

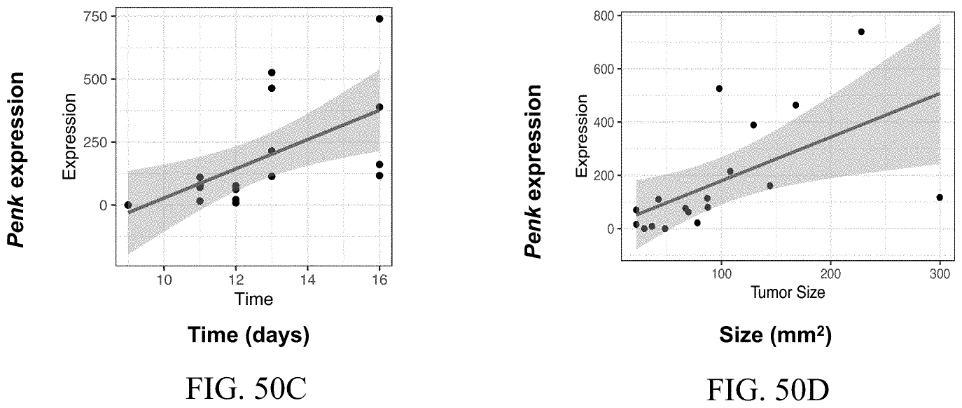

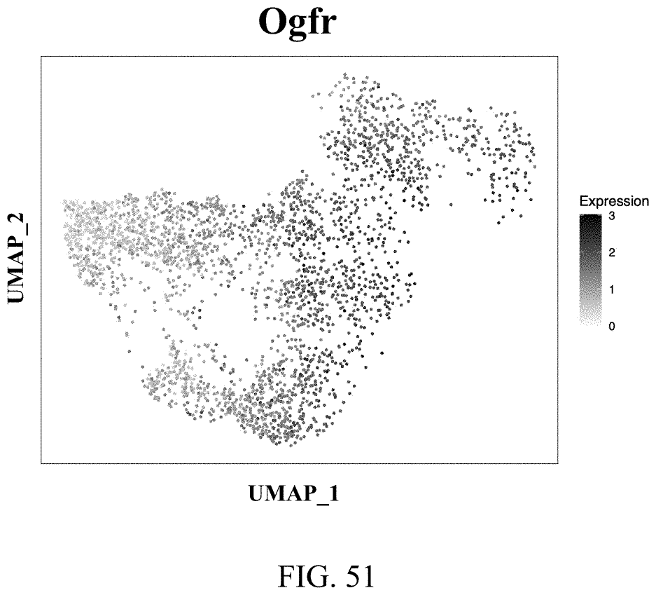

Applicants further analyzed the annotated T cell clusters (FIGS. 23A and 49A). Penk is expressed in both proliferating CD8 PD1+ TIM3+ and CD8 PD1+ TIM3+ T cells (FIG. 49B, C). PENK, is an endogenous opioid-polypeptide hormone which, via proteolytic cleavage, produces the enkephalin peptides [Met]-enk and [Leu]-enk in a 4:1 ratio. Enkephalins are found at high levels in the brain and endocrine tissues, however, studies have shown that enkephalins play an important role in behavior, pain, cardiac function, cellular growth, immunity, and ischemic tolerance. Applicants show that proenkephalin (PENK) increases over time and size in CD8+PD1+ TIM3+ cells (FIG. 50A-D). PENK is cleaved to make Met enkephalin (MENK) which can bind canonical cell-membrane opioid receptors (m, d and k). MENK is a potent agonist of the d-opioid receptor, and to a lesser extent the μ-opioid receptor, with little to no effect on the κ-opioid receptor. It is through these receptors that met-enkephalin produces its opioid effects, such as analgesia and antidepressant-like effects. In the scRNAseq dataset presented herein, the expression these receptors is not detected. Applicants show that opioid growth factor receptor is expressed in all T cells (FIG. 51). MENK can also bind the opioid growth factor receptor (OGFR) and this is why MENK is also called OGF. OGFR is widely expressed in the scRNAseq dataset and it's function in CD8+ T cells was largely unexplored before the present invention.

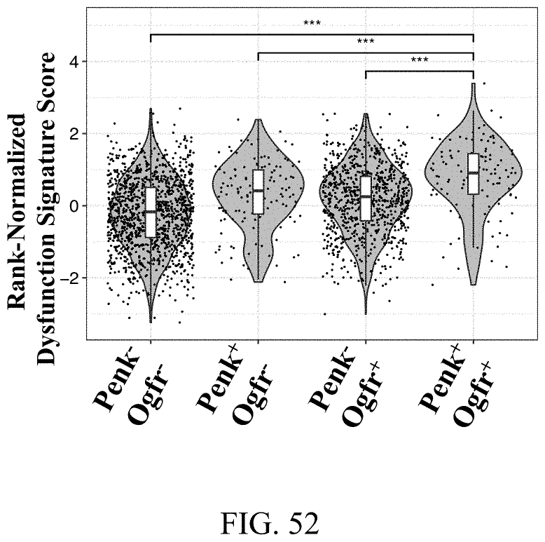

Applicants scored the single cells for a dysfunction signature (FIG. 52). PENK needs to be cleaved to release biologically active peptides. CD8+ TILs express some of the peptidases, such as Rnpep, Ctsl, and Furin. Interestingly, Lnpep, a peptidase known to cleave and inactivate MENK, is down-regulated over time and size in CD8+PD1+ TIM3+ cells. CD8 T cell-derived MENK may act both in an autocrine- or paracrine-manner. Applicants found that CD8+PD1+ TIM3+ expressing both PENK and OGFR displayed the highest dysfunction signature.

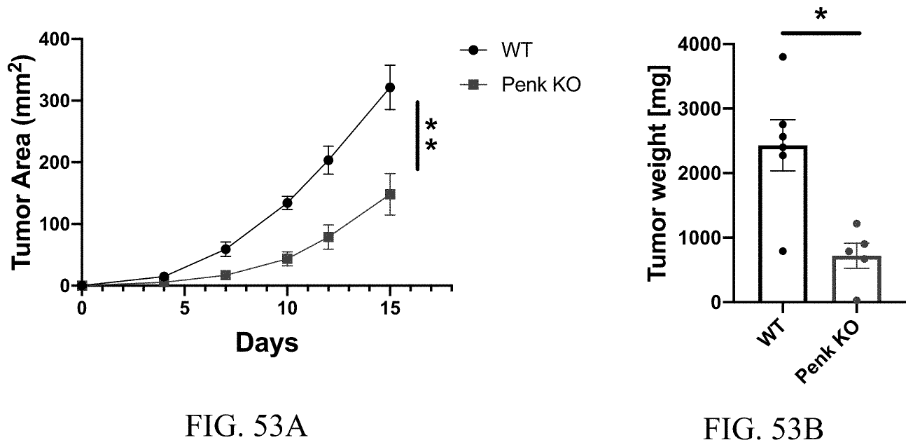

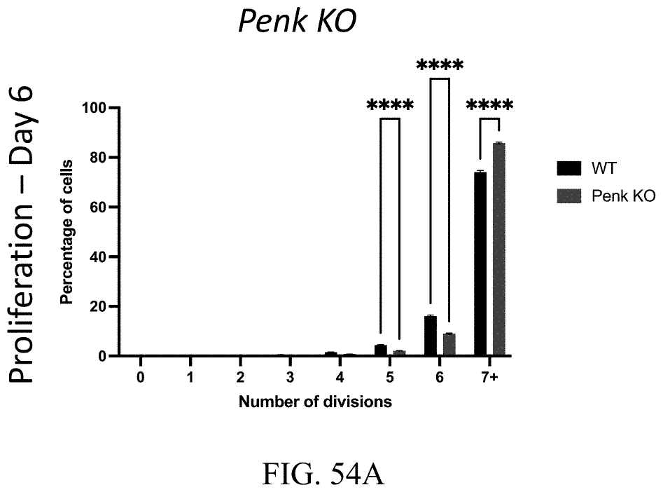

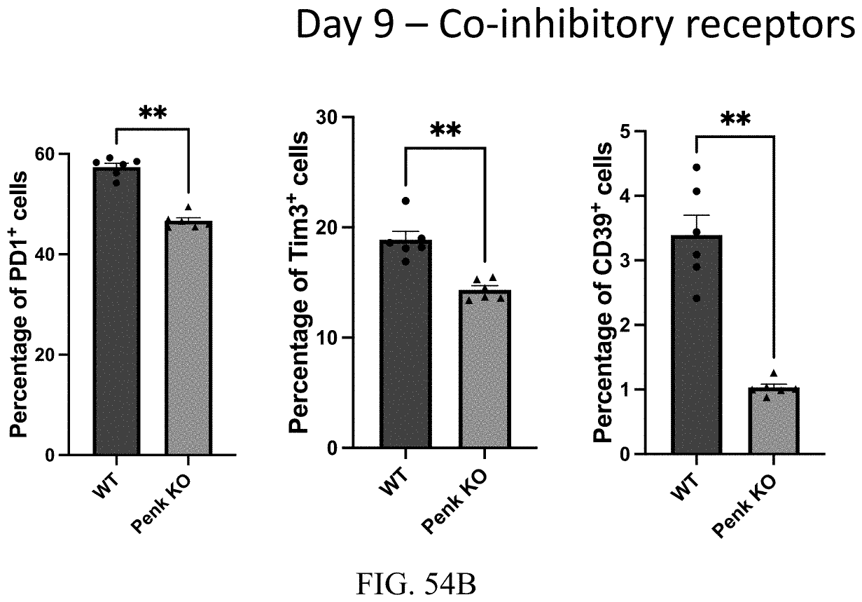

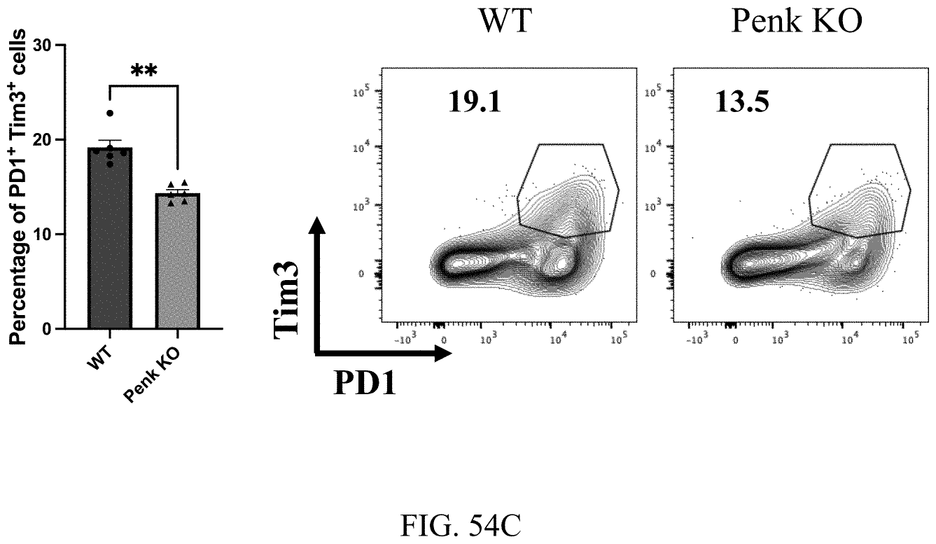

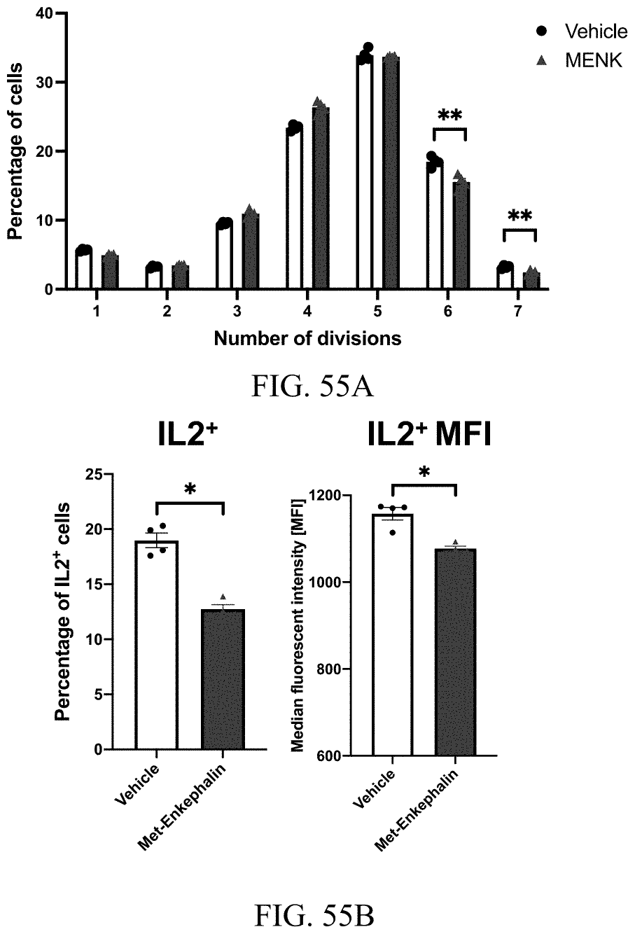

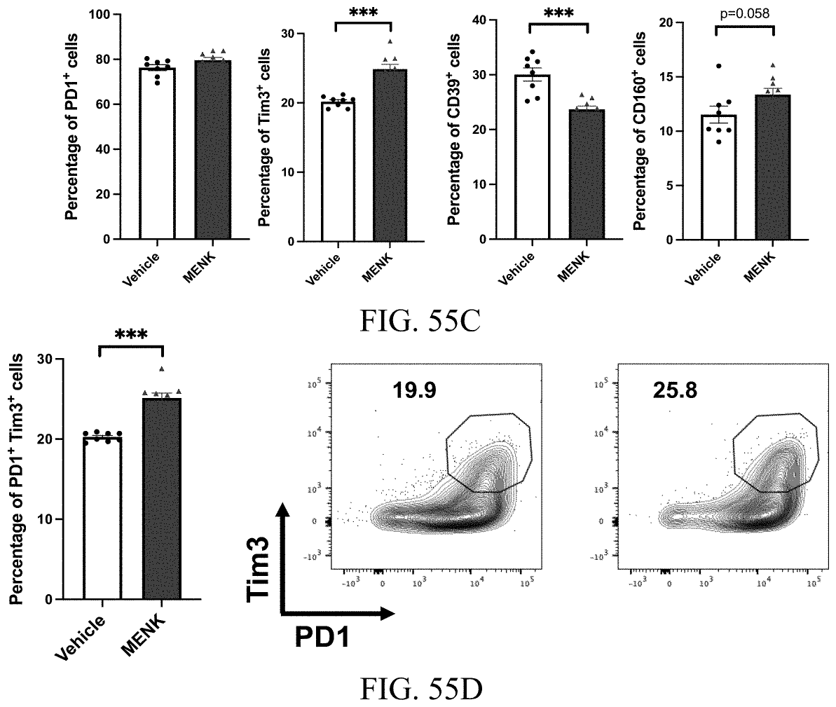

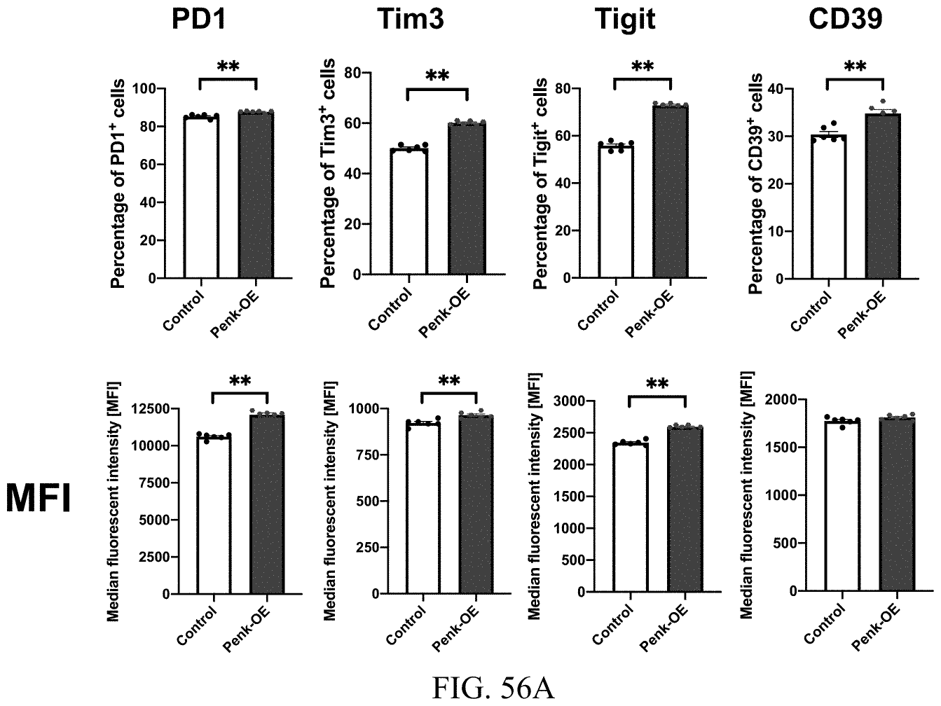

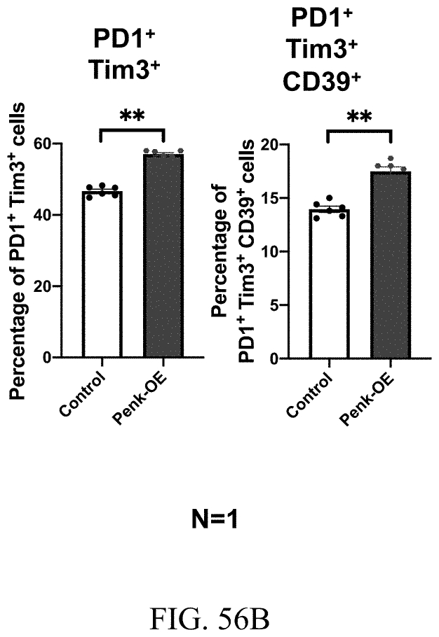

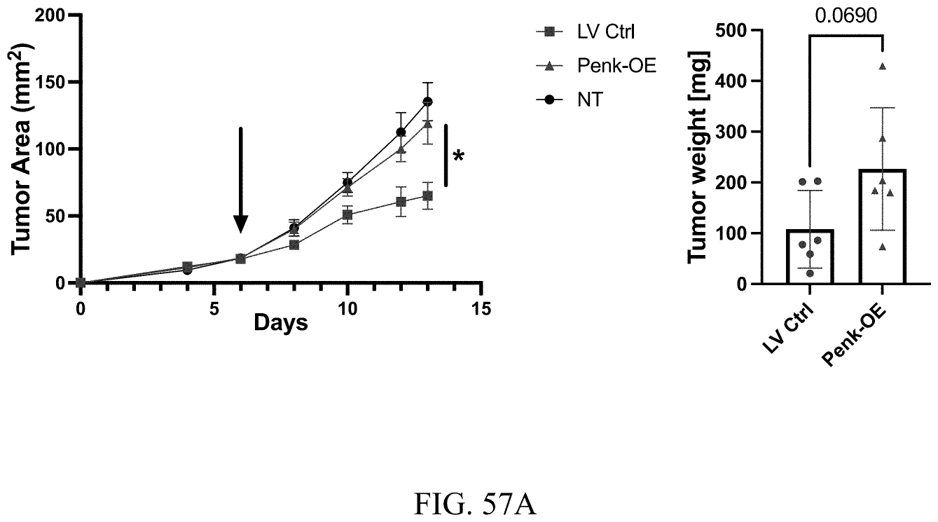

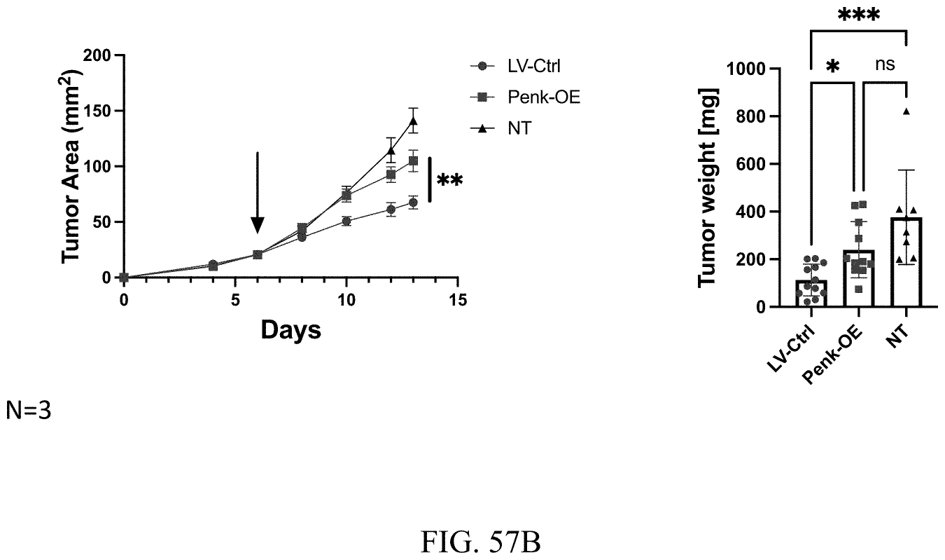

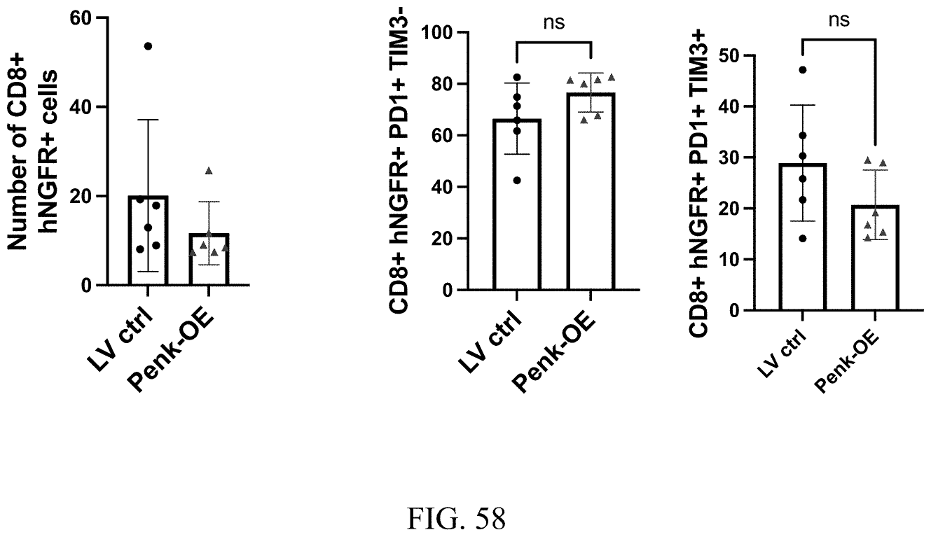

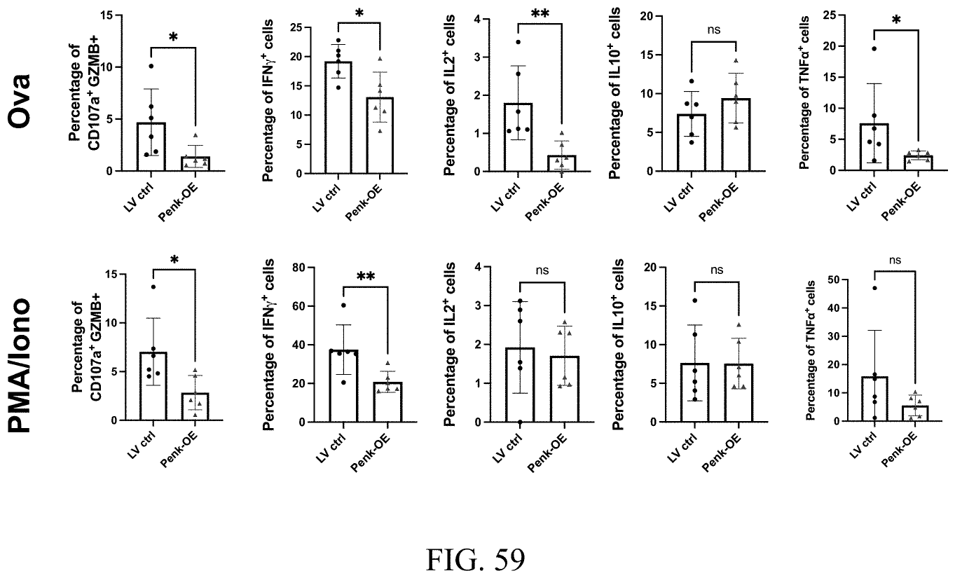

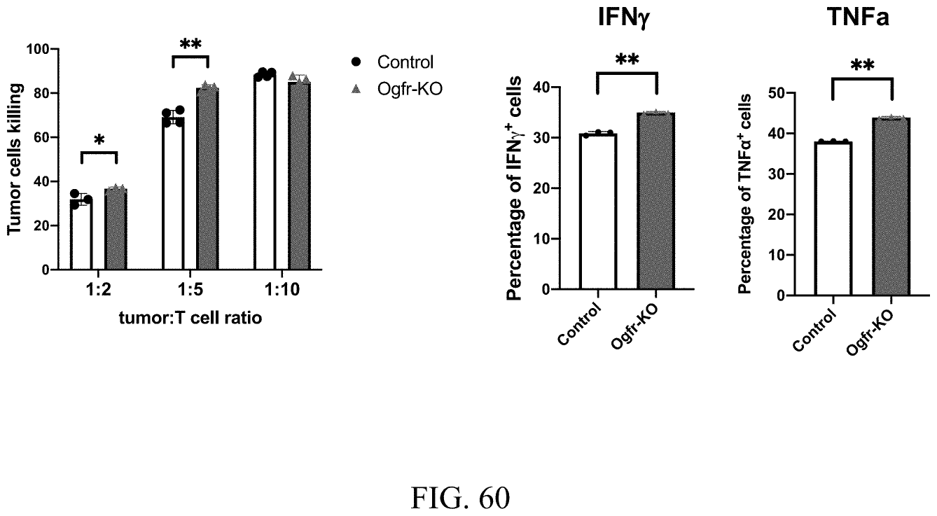

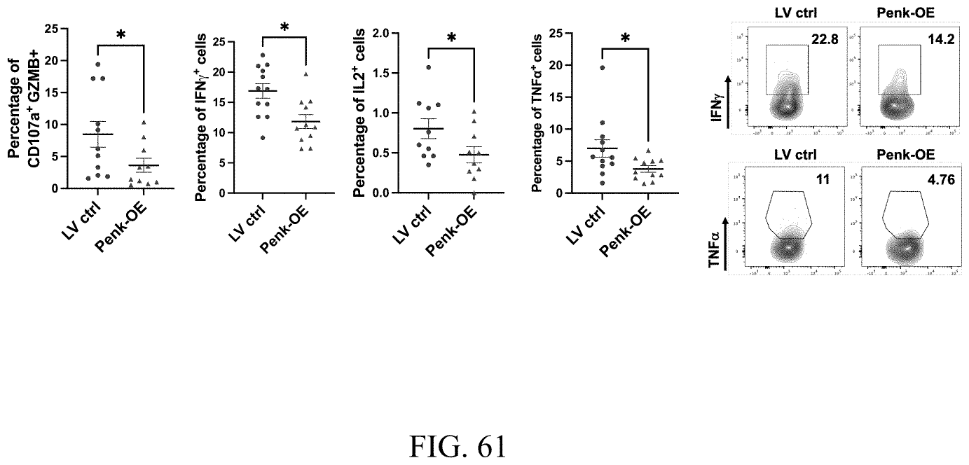

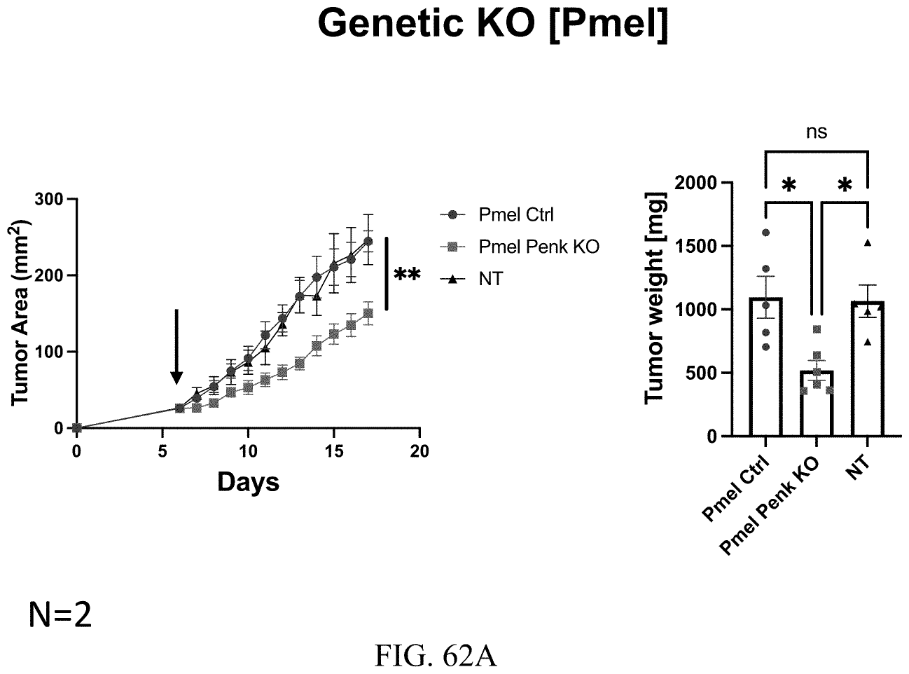

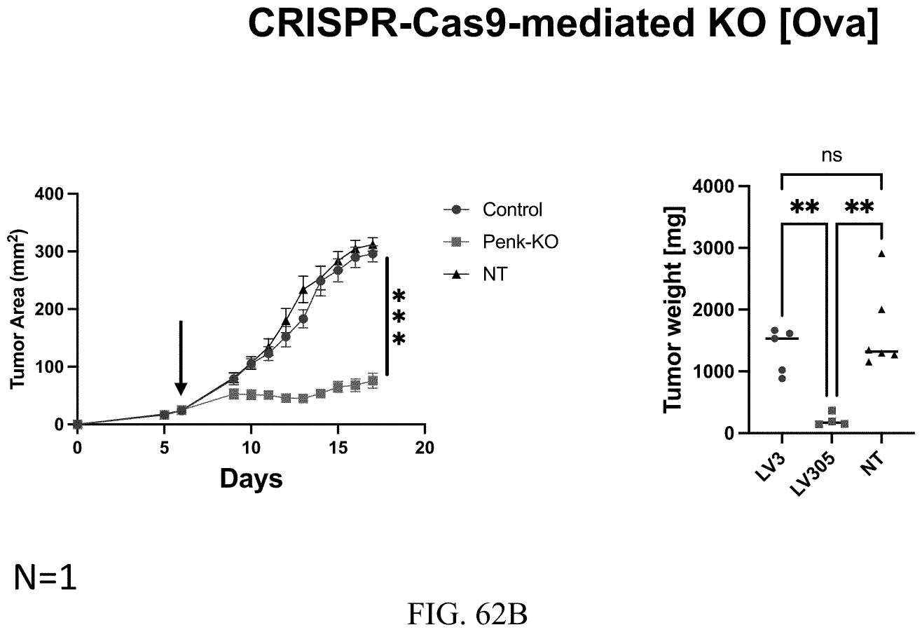

Applicants determined the effect of PENK-OGFR signaling on tumor growth and anti-tumor immunity. PENK knockout mice had delayed tumor growth in a mouse tumor model (FIG. 53A, B). Penk KO in T cells in vitro showed increased proliferation and a decreased expression of co-inhibitory receptors (FIG. 54A-C). In vitro stimulation of opioid signaling using MENK peptide showed decreased proliferation, increased PD1, TIM3 and CD160, decreased IL-2 and increased PD1+ TIM3+CD8+ T cells (FIG. 55A-D). PENK overexpression in T cells showed increased PD-1, TIM3, TIGIT and CD39 expression, as well as, increased PD1+ TIM3+ and PD1+ TIM3+CD39+ T cells (FIG. 56A, B). PENK overexpression in a mouse tumor model showed increased tumor growth (FIG. 57A, B). PENK overexpression also showed a decrease in nerve growth factor receptor (NGFR) positive CD8+ T cells (FIG. 58). PENK overexpression also showed a decrease in pro-inflammatory cytokine production and increase in IL-10 by intra-cellular cytokine (ICC) staining (FIG. 59). PENK overexpression also showed a decrease in cytolytic capacity (Cd107a+Gzmb+ cells) and capacity to produce effector (Ifng and Tnfa) or pro-survival (IL2) factors using by intra-cellular cytokine (ICC) staining (FIG. 61). Additionally, T cells that do not express Ogfr have increased tumor killing and increases in TNFa and IFNg-producing cells (FIG. 60). Finally, CD8 T cell specific Penk knockout results in decreased tumor growth in adoptive cell transfer experiments as compared to control and no transfer (FIG. 62A, B).

In summary, the examples show: 1) that Penk expression increases with time and size in CD8+ TILs that exhibit a dysfunctional phenotype in melanoma tumors, 2) improved tumor growth control in germline Penk KO mice, 3) that Penk KO CD8+ T cells proliferate more vigorously and show less checkpoint receptor expression in vitro, 4) that stimulation of CD8+ T cells in the presence of Menk (OGF) in vitro leads to some reduction in proliferation, increased expression of several checkpoints, and dampened IL-2 expression, 5) that Penk overexpression in CD8+ T cells in vitro promotes higher checkpoint receptor expression, 6) that Penk overexpression in tumor-antigen specific CD8+ T cells in vivo results in reduced cytotoxic capacity, pro-inflammatory cytokine production, and abrogates tumor growth control, and 7) that loss of OGFR in CD8+ T cells in vitro improves cytotoxicity and frequency of TNFa and IFNg-producing cells.

### Tables

TABLE 1
Ligand-Receptor Interactions. T_X indicates T cell clusters (see, FIG. 23) and M_X
indicates the non-T cell clusters (see, FIG. 25). For example T_X2 indicates the Treg cluster.

| pair | l_clusters | r_clusters | mult.score.general |
|---|---|---|---|
| XCL1_XCR1 | T_X0:T_X4:T_X7:T_X9:M_X2 | M_X10 | Inf |
| VIM_CD44 | T_X2:T_X6:T_X8:M_X1:M_X4:M_X5:M_{—} | T_X6:M_X1:M_X3:M_X5:M_X8:M_X9 | Inf |
| | X6:M_X8:M_X11 | | |
| VCAN_TLR2 | M_X1:M_X6 | M_X1:M_X5 | Inf |
| VCAN_SELL | M_X1:M_X6 | T_X0:T_X3:M_X2:M_X3:M_X11 | Inf |
| VCAN_ITGB1 | M_X1:M_X6 | T_X1:T_X5:T_X6:M_X2:M_X7 | Inf |
| VCAN_ITGA4 | M_X1:M_X6 | T_X1:M_X7:M_X9 | Inf |
| VCAN_CD44 | M_X1:M_X6 | T_X6:M_X1:M_X3:M_X5:M_X8:M_X9 | Inf |
| TRF_TFRC | M_X1:M_X5 | T_X4:T_X9:M_X3 | Inf |
| TNFSF9_TNFRSF9 | M_X4:M_X7:M_X11 | T_X2:T_X7:T_X8:T_X9:M_X2:M_X6:M_{—} | Inf |
| | | X7 | |
| TNFSF4_TNFRSF4 | M_X7 | T_X2:T_X8:T_X9:M_X4:M_X6:M_X7 | Inf |
| TNFRSF9_TNFSF9 | T_X2:T_X7:T_X8:T_X9:M_X2:M_X6:M_{—} | M_X4:M_X7:M_X11 | Inf |
| | X7 | | |
| TNFRSF4_TNFSF4 | T_X2:T_X8:T_X9:M_X4:M_X6:M_X7 | M_X7 | Inf |
| TLR2_VCAN | M_X1:M_X5 | M_X1:M_X6 | Inf |
| TIMP2_ITGB1 | T_X2:M_X3:M_X8 | T_X1:T_X5:T_X6:M_X2:M_X7 | Inf |
| TIMP1_CD63 | M_X4 | M_X7:M_X8 | Inf |
| THBS1_CD47 | M_X1:M_X6:M_X8 | T_X6:M_X3:M_X7:M_X11 | Inf |
| SLPI_CD4 | M_X1:M_X3:M_X5:M_X8 | T_X2:T_X5:T_X6:M_X3 | Inf |
| SERPINE2_LRP1 | T_X4:T_X7 | M_X1:M_X5:M_X6:M_X8:M_X9 | Inf |
| SELPLG_SELL | T_X1:M_X2:M_X3:M_X7:M_X11 | T_X0:T_X3:M_X2:M_X3:M_X11 | Inf |
| SELPLG_ITGB2 | T_X1:M_X2:M_X3:M_X7:M_X11 | T_X7:M_X1:M_X9 | Inf |
| SELPLG_ITGAM | T_X1:M_X2:M_X3:M_X7:M_X11 | M_X1:M_X6:M_X8 | Inf |
| SELL_SELPLG | T_X0:T_X3:M_X2:M_X3:M_X11 | T_X1:M_X2:M_X3:M_X7:M_X11 | Inf |
| RTN4_NGFR | M_X1:M_X7 | M_X11 | Inf |
| RPS19_C5AR1 | T_X3:M_X0:M_X7:M_X12 | M_X1:M_X6:M_X8:M_X9 | Inf |
| QRFP_P2RY14 | T_X0:M_X2 | M_X3:M_X10 | Inf |
| PSAP_LRP1 | M_X1:M_X3:M_X5:M_X10 | M_X1:M_X5:M_X6:M_X8:M_X9 | Inf |
| PDCD1_PDCD1LG2 | T_X4:T_X7:T_X9:M_X6:M_X7 | T_X2:T_X9:M_X7 | Inf |
| P2RY14_QRFP | M_X3:M_X10 | T_X0:M_X2 | Inf |
| OSM_LIFR | M_X1:M_X5:M_X6:M_X8 | M_X3:M_X11 | Inf |
| LTB_TNFRSF1A | T_X2:T_X5:M_X0:M_X2:M_X6 | M_X1:M_X5:M_X9 | Inf |
| LTB_LTBR | T_X2:T_X5:M_X0:M_X2:M_X6 | M_X1:M_X5 | Inf |
| LTB_CD40 | T_X2:T_X5:M_X0:M_X2:M_X6 | M_X1:M_X4:M_X7 | Inf |
| LRP1_SERPINE2 | M_X1:M_X5:M_X6:M_X8:M_X9 | T_X4:T_X7 | Inf |
| LRP1_PSAP | M_X1:M_X5:M_X6:M_X8:M_X9 | M_X1:M_X3:M_X5:M_X10 | Inf |
| LIFR_OSM | M_X3:M_X11 | M_X1:M_X5:M_X6:M_X8 | Inf |
| ITGB1_VCAN | T_X1:T_X5:T_X6:M_X2:M_X7 | M_X1:M_X6 | Inf |
| ITGB1_TIMP2 | T_X1:T_X5:T_X6:M_X2:M_X7 | T_X2:M_X3:M_X8 | Inf |
| ITGA4_VCAN | T_X1:M_X7:M_X9 | M_X1:M_X6 | Inf |
| IL1RN_IL1R2 | M_X1:M_X6:M_X8 | T_X2:T_X7:T_X8:M_X4 | Inf |
| IL1R2_IL1RN | T_X2:T_X7:T_X8:M_X4 | M_X1:M_X6:M_X8 | Inf |
| IL1B_IL1R2 | M_X1:M_X4:M_X5:M_X6:M_X9 | T_X2:T_X7:T_X8:M_X4 | Inf |
| IL1B_ADRB2 | M_X1:M_X4:M_X5:M_X6:M_X9 | T_X3:M_X0:M_X9 | Inf |
| IL16_CD4 | M_X0:M_X2 | T_X2:T_X5:T_X6:M_X3 | Inf |
| IL15_IL2RB | M_X1:M_X7 | T_X0:T_X2:T_X7:M_X2 | Inf |
| IL10_IL10RB | T_X2 | T_X0:M_X2 | Inf |
| IL10_IL10RA | T_X2 | T_X7 | Inf |
| IFNG_IFNGR2 | T_X4:T_X7:T_X9:M_X2 | T_X3:M_X1:M_X5 | Inf |
| IFNG_IFNGR1 | T_X4:T_X7:T_X9:M_X2 | T_X0:T_X2:T_X5:M_X2 | Inf |
| ICOS_ICOSL | T_X2:T_X5:T_X6:M_X6 | M_X7 | Inf |
| HSP90B1_TLR7 | T_X4:T_X8:T_X9:M_X3 | M_X3:M_X9 | Inf |
| HSP90B1_TLR2 | T_X4:T_X8:T_X9:M_X3 | M_X1:M_X5 | Inf |
| HSP90B1_LRP1 | T_X4:T_X8:T_X9:M_X3 | M_X1:M_X5:M_X6:M_X8:M_X9 | Inf |
| HP_ITGB2 | M_X1:M_X5:M_X9 | T_X7:M_X1:M_X9 | Inf |
| HP_ITGAM | M_X1:M_X5:M_X9 | M_X1:M_X6:M_X8 | Inf |
| HMGB1_THBD | T_X4:T_X8:M_X2:M_X3 | M_X5 | Inf |
| HMGB1_SDC1 | T_X4:T_X8:M_X2:M_X3 | M_X8 | Inf |
| H2-M3_KLRD1 | M_X3 | T_X0:T_X1:T_X4:T_X7:M_X2:M_X3:M_{—} | Inf |
| | | X4:M_X11 | |
| H2-M3_KLRC1 | M_X3 | T_X1:T_X4:T_X7:T_X9:M_X2 | Inf |
| H2-M3_CD4 | M_X3 | T_X2:T_X5:T_X6:M_X3 | Inf |
| GNAI2_C5AR1 | T_X2:T_X8:M_X4:M_X11 | M_X1:M_X6:M_X8:M_X9 | Inf |
| FN1_ITGB7 | M_X1:M_X5:M_X6:M_X8 | T_X2:T_X6:M_X4:M_X10 | Inf |
| FN1_CD79A | M_X1:M_X5:M_X6:M_X8 | M_X0:M_X12 | Inf |
| FN1_C5AR1 | M_X1:M_X5:M_X6:M_X8 | M_X1:M_X6:M_X8:M_X9 | Inf |
| FASL_TNFRSF1A | T_X1:M_X2 | M_X1:M_X5:M_X9 | Inf |
| FASL_FAS | T_X1:M_X2 | M_X1:M_X7 | Inf |
| FAS_FASL | M_X1:M_X7 | T_X1:M_X2 | Inf |
| F13A1_ITGB1 | M_X1:M_X5:M_X6:M_X8 | T_X1:T_X5:T_X6:M_X2:M_X7 | Inf |
| F13A1_ITGA4 | M_X1:M_X5:M_X6:M_X8 | T_X1:M_X7:M_X9 | Inf |
| CXCL2_XCR1 | M_X1:M_X6:M_X8:M_X9 | M_X10 | Inf |
| CXCL16_CXCR6 | M_X4:M_X7:M_X8 | T_X1:T_X4:T_X6:T_X7:M_X6 | Inf |
| CSF1_CSF1R | T_X7 | M_X1:M_X5:M_X6:M_X9 | Inf |
| CDH5_CDH5 | M_X3 | M_X3 | Inf |
| CDH1_KLRG1 | M_X3 | T_X2:T_X8 | Inf |
| CDH1_ITGB7 | M_X3 | T_X2:T_X6:M_X4:M_X10 | Inf |
| CDH1_ITGAE | M_X3 | T_X2:M_X10 | Inf |
| CD86_CTLA4 | M_X1:M_X4:M_X7:M_X8 | T_X2:T_X8:M_X6 | Inf |
| CD80_CTLA4 | M_X7 | T_X2:T_X8:M_X6 | Inf |
| CD80_CD28 | M_X7 | T_X1:T_X5:T_X6:M_X2:M_X6 | Inf |
| CD63_TIMP1 | M_X7:M_X8 | M_X4 | Inf |
| CD47_THBS1 | T_X6:M_X3:M_X7:M_X11 | M_X1:M_X6:M_X8 | Inf |
| CD44_VIM | T_X6:M_X1:M_X3:M_X5:M_X8:M_X9 | T_X2:T_X6:T_X8:M_X1:M_X4:M_X5:M_{—} | Inf |
| | | X6:M_X8:M_X11 | |
| CD44_VCAN | T_X6:M_X1:M_X3:M_X5:M_X8:M_X9 | M_X1:M_X6 | Inf |
| CD40_LTB | M_X1:M_X4:M_X7 | T_X2:T_X5:M_X0:M_X2:M_X6 | Inf |
| CD4_IL16 | T_X2:T_X5:T_X6:M_X3 | M_X0:M_X2 | Inf |
| CD4_H2-DMA | T_X2:T_X5:T_X6:M_X3 | M_X4:M_X10:M_X11 | Inf |
| CD28_CD86 | T_X1:T_X5:T_X6:M_X2:M_X6 | M_X1:M_X4:M_X7:M_X8 | Inf |
| CD28_CD80 | T_X1:T_X5:T_X6:M_X2:M_X6 | M_X7 | Inf |
| CD274_PDCD1 | M_X7:M_X8 | T_X4:T_X7:T_X9:M_X6:M_X7 | Inf |
| CD14_ITGB1 | M_X1:M_X5:M_X6:M_X8 | T_X1:T_X5:T_X6:M_X2:M_X7 | Inf |
| CD14_ITGA4 | M_X1:M_X5:M_X6:M_X8 | T_X1:M_X7:M_X9 | Inf |
| CCR5_CCL4 | T_X2:M_X1:M_X6:M_X8 | T_X4:T_X7:T_X9:M_X2:M_X3 | Inf |
| CCL8_CCR2 | M_X8 | T_X2:T_X6:M_X1:M_X2:M_X5:M_X6 | Inf |
| CCL8_CCR1 | M_X8 | T_X2:M_X1:M_X6:M_X8 | Inf |
| CCL7_CXCR3 | M_X1:M_X6:M_X8 | T_X1:T_X2:T_X5:T_X6:M_X2:M_X3 | Inf |
| CCL7_CCR5 | M_X1:M_X6:M_X8 | T_X2:M_X1:M_X6:M_X8 | Inf |
| CCL7_CCR2 | M_X1:M_X6:M_X8 | T_X2:T_X6:M_X1:M_X2:M_X5:M_X6 | Inf |
| CCL7_CCR1 | M_X1:M_X6:M_X8 | T_X2:M_X1:M_X6:M_X8 | Inf |
| CCL5_SDC4 | T_X0:T_X1:M_X2:M_X7 | T_X2:M_X1:M_X3 | Inf |
| CCL5_SDC1 | T_X0:T_X1:M_X2:M_X7 | M_X8 | Inf |
| CCL5_CXCR3 | T_X0:T_X1:M_X2:M_X7 | T_X1:T_X2:T_X5:T_X6:M_X2:M_X3 | Inf |
| CCL5_CCR5 | T_X0:T_X1:M_X2:M_X7 | T_X2:M_X1:M_X6:M_X8 | Inf |
| CCL5_CCR1 | T_X0:T_X1:M_X2:M_X7 | T_X2:M_X1:M_X6:M_X8 | Inf |
| CCL4_CCR8 | T_X4:T_X7:T_X9:M_X2:M_X3 | T_X2 | Inf |
| CCL4_CCR5 | T_X4:T_X7:T_X9:M_X2:M_X3 | T_X2:M_X1:M_X6:M_X8 | Inf |
| CCL4_CCR1 | T_X4:T_X7:T_X9:M_X2:M_X3 | T_X2:M_X1:M_X6:M_X8 | Inf |
| CCL3_CCR5 | T_X4:T_X7:T_X9:M_X1:M_X2:M_X8:M_X9 | T_X2:M_X1:M_X6:M_X8 | Inf |
| CCL3_CCR1 | T_X4:T_X7:T_X9:M_X1:M_X2:M_X8:M_X9 | T_X2:M_X1:M_X6:M_X8 | Inf |
| CCL24_CCR2 | M_X6 | T_X2:T_X6:M_X1:M_X2:M_X5:M_X6 | Inf |
| CCL22_DPP4 | M_X7 | M_X4:M_X7:M_X10:M_X11 | Inf |
| CCL2_CCR5 | M_X1:M_X6:M_X8 | T_X2:M_X1:M_X6:M_X8 | Inf |
| CCL2_CCR2 | M_X1:M_X6:M_X8 | T_X2:T_X6:M_X1:M_X2:M_X5:M_X6 | Inf |
| CCL2_CCR1 | M_X1:M_X6:M_X8 | T_X2:M_X1:M_X6:M_X8 | Inf |
| CALM1_HMMR | T_X4:T_X8:M_X7:M_X10 | T_X4:T_X8 | Inf |
| CADM1_CRTAM | M_X10 | T_X7:T_X9 | Inf |
| CADM1_CADM1 | M_X10 | M_X10 | Inf |
| C5AR1_RPS19 | M_X1:M_X6:M_X8:M_X9 | T_X3:M_X0:M_X7:M_X12 | Inf |
| C5AR1_GNAI2 | M_X1:M_X6:M_X8:M_X9 | T_X2:T_X8:M_X4:M_X11 | Inf |
| C3_IFITM1 | M_X1:M_X5 | M_X4:M_X10:M_X11 | Inf |
| C3_CD81 | M_X1:M_X5 | T_X2:T_X8:M_X0:M_X12 | Inf |
| C3_CD19 | M_X1:M_X5 | M_X0:M_X12 | Inf |
| C3_C3AR1 | M_X1:M_X5 | M_X1:M_X6:M_X8 | Inf |
| C1QB_LRP1 | M_X1:M_X6:M_X8 | M_X1:M_X5:M_X6:M_X8:M_X9 | Inf |
| BTLA_CD79A | M_X0:M_X10:M_X11 | M_X0:M_X12 | Inf |
| BTLA_CD247 | M_X0:M_X10:M_X11 | M_X2:M_X6 | Inf |
| B2M_TFRC | T_X2:M_X6:M_X7:M_X9 | T_X4:T_X9:M_X3 | Inf |
| B2M_KLRD1 | T_X2:M_X6:M_X7:M_X9 | T_X0:T_X1:T_X4:T_X7:M_X2:M_X3:M_{—} | Inf |
| | | X4:M_X11 | |
| B2M_KLRC1 | T_X2:M_X6:M_X7:M_X9 | T_X1:T_X4:T_X7:T_X9:M_X2 | Inf |
| B2M_HFE | T_X2:M_X6:M_X7:M_X9 | M_X4:M_X5:M_X7:M_X9:M_X10 | Inf |
| B2M_CD3G | T_X2:M_X6:M_X7:M_X9 | T_X7:M_X6 | Inf |
| B2M_CD3D | T_X2:M_X6:M_X7:M_X9 | T_X7:M_X6 | Inf |
| B2M_CD247 | T_X2:M_X6:M_X7:M_X9 | M_X2:M_X6 | Inf |
| APP_TNFRSF21 | M_X1:M_X5:M_X6:M_X8 | M_X1:M_X5 | Inf |
| APP_NGFR | M_X1:M_X5:M_X6:M_X8 | M_X11 | Inf |
| APP_LRP1 | M_X1:M_X5:M_X6:M_X8 | M_X1:M_X5:M_X6:M_X8:M_X9 | Inf |
| APP_CD74 | M_X1:M_X5:M_X6:M_X8 | T_X2:T_X8:M_X0:M_X4:M_X7:M_X10 | Inf |
| APOE_SORL1 | M_X1:M_X5:M_X6:M_X8:M_X9 | M_X9 | Inf |
| APOE_SCARB1 | M_X1:M_X5:M_X6:M_X8:M_X9 | M_X5:M_X11 | Inf |
| APOE_LRP8 | M_X1:M_X5:M_X6:M_X8:M_X9 | M_X3 | Inf |
| APOE_LRP1 | M_X1:M_X5:M_X6:M_X8:M_X9 | M_X1:M_X5:M_X6:M_X8:M_X9 | Inf |
| ALCAM_CD6 | T_X2:T_X9:M_X4:M_X11 | T_X2:T_X6:M_X6 | Inf |
| ADRB2_IL1B | T_X3:M_X0:M_X9 | M_X1:M_X4:M_X5:M_X6:M_X9 | Inf |
| FN1_ITGB8 | M_X1:M_X5:M_X6:M_X8 | T_X2 | 74254.31 |
| CALM3_SELL | T_X4:T_X7:T_X8:M_X7 | T_X0:T_X3:M_X2:M_X3:M_X11 | 67024.24 |
| IL18_IL18R1 | M_X1 | T_X6:M_X2 | 63600.53 |
| FN1_PLAUR | M_X1:M_X5:M_X6:M_X8 | M_X1:M_X3:M_X5:M_X8:M_X9 | 63175.69 |
| PTPRC_CD22 | M_X2:M_X9 | M_X0 | 56364.05 |
| CD22_PTPRC | M_X0 | M_X2:M_X9 | 56364.05 |
| FN1_ITGB1 | M_X1:M_X5:M_X6:M_X8 | T_X1:T_X5:T_X6:M_X2:M_X7 | 56109.13 |
| CALM1_SELL | T_X4:T_X8:M_X7:M_X10 | T_X0:T_X3:M_X2:M_X3:M_X11 | 53276.30 |
| THBS1_SDC4 | M_X1:M_X6:M_X8 | T_X2:M_X1:M_X3 | 52138.59 |
| F10_ITGAM | M_X1:M_X5:M_X6:M_X8:M_X9 | M_X1:M_X6:M_X8 | 51971.38 |
| SEMA7A_ITGB1 | M_X7 | T_X1:T_X5:T_X6:M_X2:M_X7 | 51903.01 |
| ITGB1_SEMA7A | T_X1:T_X5:T_X6:M_X2:M_X7 | M_X7 | 51903.01 |
| CXCL10_SDC4 | M_X1:M_X4:M_X6 | T_X2:M_X1:M_X3 | 50598.07 |
| FN1_CD44 | M_X1:M_X5:M_X6:M_X8 | T_X6:M_X1:M_X3:M_X5:M_X8:M_X9 | 50426.33 |
| CD44_FN1 | T_X6:M_X1:M_X3:M_X5:M_X8:M_X9 | M_X1:M_X5:M_X6:M_X8 | 50426.33 |
| SPP1_ITGB1 | T_X4:T_X7:M_X1:M_X6:M_X8 | T_X1:T_X5:T_X6:M_X2:M_X7 | 50066.54 |
| ITGB1_SPP1 | T_X1:T_X5:T_X6:M_X2:M_X7 | T_X4:T_X7:M_X1:M_X6:M_X8 | 50066.54 |
| FN1_ITGAV | M_X1:M_X5:M_X6:M_X8 | T_X2 | 48656.13 |
| THBS1_ITGB1 | M_X1:M_X6:M_X8 | T_X1:T_X5:T_X6:M_X2:M_X7 | 48654.63 |
| ITGB1_THBS1 | T_X1:T_X5:T_X6:M_X2:M_X7 | M_X1:M_X6:M_X8 | 48654.63 |
| CD40LG_ITGAM | T_X5:T_X6 | M_X1:M_X6:M_X8 | 47121.32 |
| TGM2_TBXA2R | M_X1:M_X6:M_X8:M_X9 | M_X3 | 45503.40 |
| SPP1_CD44 | T_X4:T_X7:M_X1:M_X6:M_X8 | T_X6:M_X1:M_X3:M_X5:M_X8:M_X9 | 44995.74 |
| CD44_SPP1 | T_X6:M_X1:M_X3:M_X5:M_X8:M_X9 | T_X4:T_X7:M_X1:M_X6:M_X8 | 44995.74 |
| F10_ITGB2 | M_X1:M_X5:M_X6:M_X8:M_X9 | T_X7:M_X1:M_X9 | 43769.00 |
| SPP1_ITGAV | T_X4:T_X7:M_X1:M_X6:M_X8 | T_X2 | 43416.19 |
| ITGAV_SPP1 | T_X2 | T_X4:T_X7:M_X1:M_X6:M_X8 | 43416.19 |
| PTPRC_MRC1 | M_X2:M_X9 | M_X6:M_X8 | 43075.70 |
| TGM2_SDC4 | M_X1:M_X6:M_X8:M_X9 | T_X2:M_X1:M_X3 | 41892.43 |
| SDC4_TGM2 | T_X2:M_X1:M_X3 | M_X1:M_X6:M_X8:M_X9 | 41892.43 |
| LY86_CD180 | M_X1:M_X3:M_X11 | M_X0:M_X3 | 41719.32 |
| CD180_LY86 | M_X0:M_X3 | M_X1:M_X3:M_X11 | 41719.32 |
| IL18_IL18RAP | M_X1 | M_X2 | 40218.96 |
| CD40LG_ITGB2 | T_X5:T_X6 | T_X7:M_X1:M_X9 | 39684.40 |
| SEMA7A_PLXNC1 | M_X7 | M_X7 | 39644.16 |
| PLXNC1_SEMA7A | M_X7 | M_X7 | 39644.16 |
| TGM2_ITGB1 | M_X1:M_X6:M_X8:M_X9 | T_X1:T_X5:T_X6:M_X2:M_X7 | 39093.14 |
| ITGB1_TGM2 | T_X1:T_X5:T_X6:M_X2:M_X7 | M_X1:M_X6:M_X8:M_X9 | 39093.14 |
| CXCL2_DPP4 | M_X1:M_X6:M_X8:M_X9 | M_X4:M_X7:M_X10:M_X11 | 34788.81 |
| MMP12_PLAUR | M_X8 | M_X1:M_X3:M_X5:M_X8:M_X9 | 32846.29 |
| CALR_ITGAV | T_X2:T_X4:T_X7:T_X8:T_X9:M_X1:M_{—} | T_X2 | 32261.62 |
| | X4:M_X10:M_X11 | | |
| SPP1_S1PR1 | T_X4:T_X7:M_X1:M_X6:M_X8 | T_X3:M_X0 | 29430.92 |
| S1PR1_SPP1 | T_X3:M_X0 | T_X4:T_X7:M_X1:M_X6:M_X8 | 29430.92 |
| TRF_GPR162 | M_X1:M_X5 | M_X3 | 29085.73 |
| CD70_CD27 | T_X7:M_X7 | T_X2:M_X2 | 28787.74 |
| CD27_CD70 | T_X2:M_X2 | T_X7:M_X7 | 28787.74 |
| LPL_CD44 | M_X1:M_X6:M_X8 | T_X6:M_X1:M_X3:M_X5:M_X8:M_X9 | 28622.36 |
| TGFB1_ITGB8 | T_X2:T_X8:T_X9:M_X1:M_X5:M_X9 | T_X2 | 28369.98 |
| ITGB8_TGFB1 | T_X2 | T_X2:T_X8:T_X9:M_X1:M_X5:M_X9 | 28369.98 |
| C3_ITGAM | M_X1:M_X5 | M_X1:M_X6:M_X8 | 28337.51 |
| THBS1_LRP1 | M_X1:M_X6:M_X8 | M_X1:M_X5:M_X6:M_X8:M_X9 | 26925.50 |
| LRP1_THBS1 | M_X1:M_X5:M_X6:M_X8:M_X9 | M_X1:M_X6:M_X8 | 26925.50 |
| LAMC1_ITGB1 | T_X2 | T_X1:T_X5:T_X6:M_X2:M_X7 | 25224.82 |
| CXCL10_CXCR3 | M_X1:M_X4:M_X6 | T_X1:T_X2:T_X5:T_X6:M_X2:M_X3 | 24130.98 |
| CALM2_SELL | T_X8:M_X2 | T_X0:T_X3:M_X2:M_X3:M_X11 | 24052.87 |
| C3_ITGB2 | M_X1:M_X5 | T_X7:M_X1:M_X9 | 23865.14 |
| CDH1_CDH1 | M_X3 | M_X3 | 23700.60 |
| CD274_CD80 | M_X7:M_X8 | M_X7 | 23029.97 |
| CCL8_CCR5 | M_X8 | T_X2:M_X1:M_X6:M_X8 | 22214.03 |
| LAMC1_ITGAV | T_X2 | T_X2 | 21874.20 |
| CD40LG_CD40 | T_X5:T_X6 | M_X1:M_X4:M_X7 | 21489.63 |
| CD40_CD40LG | M_X1:M_X4:M_X7 | T_X5:T_X6 | 21489.63 |
| THBS1_CD36 | M_X1:M_X6:M_X8 | M_X6:M_X8:M_X9 | 21098.38 |
| CD36_THBS1 | M_X6:M_X8:M_X9 | M_X1:M_X6:M_X8 | 21098.38 |
| IL15_IL2RA | M_X1:M_X7 | T_X2:T_X8:T_X9:M_X7 | 20889.39 |
| CCL1_CCR8 | T_X4:T_X7:T_X9 | T_X2 | 20869.77 |
| CALR_LRP1 | T_X2:T_X4:T_X7:T_X8:T_X9:M_X1:M_{—} | M_X1:M_X5:M_X6:M_X8:M_X9 | 20588.35 |
| | X4:M_X10:M_X11 | | |
| IL18_CD48 | M_X1 | T_X1:T_X6:T_X7:M_X4:M_X11 | 18896.98 |
| TGFB1_ITGAV | T_X2:T_X8:T_X9:M_X1:M_X5:M_X9 | T_X2 | 18589.81 |
| ITGAV_TGFB1 | T_X2 | T_X2:T_X8:T_X9:M_X1:M_X5:M_X9 | 18589.81 |
| C3_ITGAX | M_X1:M_X5 | M_X4:M_X11 | 18384.81 |
| CXCL9_DPP4 | M_X1:M_X4:M_X6 | M_X4:M_X7:M_X10:M_X11 | 18063.06 |
| ADAM9_ITGB1 | M_X1 | T_X1:T_X5:T_X6:M_X2:M_X7 | 17973.72 |
| LPL_LRP1 | M_X1:M_X6:M_X8 | M_X1:M_X5:M_X6:M_X8:M_X9 | 17624.68 |
| CALM1_KCNN4 | T_X4:T_X8:M_X7:M_X10 | T_X6:M_X4:M_X11 | 17430.67 |
| C3_LRP1 | M_X1:M_X5 | M_X1:M_X5:M_X6:M_X8:M_X9 | 17130.73 |
| CALM1_FAS | T_X4:T_X8:M_X7:M_X10 | M_X1:M_X7 | 17048.98 |
| ADAM15_ITGB1 | M_X1:M_X5 | T_X1:T_X5:T_X6:M_X2:M_X7 | 16948.85 |
| LGALS3BP_ITGB1 | T_X6:M_X1:M_X6 | T_X1:T_X5:T_X6:M_X2:M_X7 | 16694.11 |
| ITGB1_LGALS3BP | T_X1:T_X5:T_X6:M_X2:M_X7 | T_X6:M_X1:M_X6 | 16694.11 |
| ICAM1_IL2RA | T_X2:T_X9:M_X1:M_X7 | T_X2:T_X8:T_X9:M_X7 | 16398.94 |
| ADAM9_ITGAV | M_X1 | T_X2 | 15586.26 |
| THBS1_SCARB1 | M_X1:M_X6:M_X8 | M_X5:M_X11 | 15337.78 |
| THBS1_SDC1 | M_X1:M_X6:M_X8 | M_X8 | 15133.27 |
| SDC1_THBS1 | M_X8 | M_X1:M_X6:M_X8 | 15133.27 |
| FN1_NT5E | M_X1:M_X5:M_X6:M_X8 | T_X2 | 14948.61 |
| FN1_ITGA4 | M_X1:M_X5:M_X6:M_X8 | T_X1:M_X7:M_X9 | 14874.73 |
| ADAM15_ITGAV | M_X1:M_X5 | T_X2 | 14697.53 |
| GNAI2_TBXA2R | T_X2:T_X8:M_X4:M_X11 | M_X3 | 13917.42 |
| LAMB3_ITGB1 | T_X1:M_X0:M_X2 | T_X1:T_X5:T_X6:M_X2:M_X7 | 13360.82 |
| SPP1_ITGA4 | T_X4:T_X7:M_X1:M_X6:M_X8 | T_X1:M_X7:M_X9 | 13272.82 |
| ITGA4_SPP1 | T_X1:M_X7:M_X9 | T_X4:T_X7:M_X1:M_X6:M_X8 | 13272.82 |
| GNAI2_CCR5 | T_X2:T_X8:M_X4:M_X11 | T_X2:M_X1:M_X6:M_X8 | 13192.03 |
| CCR5_GNAI2 | T_X2:M_X1:M_X6:M_X8 | T_X2:T_X8:M_X4:M_X11 | 13192.03 |
| THBS1_ITGA4 | M_X1:M_X6:M_X8 | T_X1:M_X7:M_X9 | 12898.52 |
| ITGA4_THBS1 | T_X1:M_X7:M_X9 | M_X1:M_X6:M_X8 | 12898.52 |
| IL15_IL15RA | M_X1:M_X7 | M_X7 | 11769.12 |
| CXCL9_CXCR3 | M_X1:M_X4:M_X6 | T_X1:T_X2:T_X5:T_X6:M_X2:M_X3 | 11586.63 |
| ICAM1_ITGAM | T_X2:T_X9:M_X1:M_X7 | M_X1:M_X6:M_X8 | 10773.89 |
| PF4_CXCR3 | M_X6:M_X8 | T_X1:T_X2:T_X5:T_X6:M_X2:M_X3 | 10613.76 |
| CXCR3_PF4 | T_X1:T_X2:T_X5:T_X6:M_X2:M_X3 | M_X6:M_X8 | 10613.76 |
| TGM2_ITGA4 | M_X1:M_X6:M_X8:M_X9 | T_X1:M_X7:M_X9 | 10363.73 |
| ANXA1_FPR2 | T_X6:M_X1:M_X5:M_X8:M_X10 | M_X1 | 10005.94 |
| LPL_SDC1 | M_X1:M_X6:M_X8 | M_X8 | 9905.82 |
| PECAM1_PECAM1 | T_X3:M_X0:M_X3 | T_X3:M_X0:M_X3 | 9801.00 |
| CFH_SELL | M_X5 | T_X0:T_X3:M_X2:M_X3:M_X11 | 9322.07 |
| TGFB1_ACVRL1 | T_X2:T_X8:T_X9:M_X1:M_X5:M_X9 | M_X4:M_X8:M_X10 | 9178.75 |
| ACVRL1_TGFB1 | M_X4:M_X8:M_X10 | T_X2:T_X8:T_X9:M_X1:M_X5:M_X9 | 9178.75 |
| CD48_CD244 | T_X1:T_X6:T_X7:M_X4:M_X11 | T_X7:M_X2:M_X5 | 9169.75 |
| VEGFA_ITGB1 | M_X8 | T_X1:T_X5:T_X6:M_X2:M_X7 | 9081.65 |
| ICAM1_ITGB2 | T_X2:T_X9:M_X1:M_X7 | T_X7:M_X1:M_X9 | 9073.50 |
| VEGFA_ITGAV | M_X8 | T_X2 | 7875.33 |
| GPI1_AMFR | T_X2:T_X7:M_X8 | M_X3 | 7743.00 |
| AMFR_GPI1 | M_X3 | T_X2:T_X7:M_X8 | 7743.00 |
| GNAI2_F2R | T_X2:T_X8:M_X4:M_X11 | M_X2 | 7562.70 |
| F2R_GNAI2 | M_X2 | T_X2:T_X8:M_X4:M_X11 | 7562.70 |
| C1QA_CD93 | M_X1:M_X6:M_X8 | M_X8 | 7470.50 |
| GNAI2_S1PR1 | T_X2:T_X8:M_X4:M_X11 | T_X3:M_X0 | 7028.64 |
| ICAM1_ITGAX | T_X2:T_X9:M_X1:M_X7 | M_X4:M_X11 | 6989.89 |
| PF4_THBD | M_X6:M_X8 | M_X5 | 6952.79 |
| TGFB1_TGFBR1 | T_X2:T_X8:T_X9:M_X1:M_X5:M_X9 | M_X3:M_X9 | 6914.19 |
| GNAI2_CXCR3 | T_X2:T_X8:M_X4:M_X11 | T_X1:T_X2:T_X5:T_X6:M_X2:M_X3 | 6110.71 |
| VEGFA_SIRPA | M_X8 | M_X1:M_X3:M_X5 | 5691.26 |
| L1CAM_ITGAV | M_X11 | T_X2 | 5597.84 |
| ITGAV_L1CAM | T_X2 | M_X11 | 5597.84 |
| CFH_ITGAM | M_X5 | M_X1:M_X6:M_X8 | 5589.41 |
| GNAI2_S1PR4 | T_X2:T_X8:M_X4:M_X11 | T_X6:M_X2:M_X3 | 5476.14 |
| TNFSF14_LTBR | T_X9 | M_X1:M_X5 | 5213.50 |
| LTBR_TNFSF14 | M_X1:M_X5 | T_X9 | 5213.50 |
| UBA52_TGFBR1 | T_X3:M_X0 | M_X3:M_X9 | 4667.20 |
| ICAM1_ITGAL | T_X2:T_X9:M_X1:M_X7 | M_X5:M_X9 | 4639.26 |
| VEGFA_NRP1 | M_X8 | T_X6:T_X7:M_X3:M_X4 | 4543.81 |
| NRP1_VEGFA | T_X6:T_X7:M_X3:M_X4 | M_X8 | 4543.81 |
| TNFSF4_TRAF2 | M_X7 | M_X7 | 4466.16 |
| VEGFA_NRP2 | M_X8 | M_X7 | 4392.05 |
| NRP2_VEGFA | M_X7 | M_X8 | 4392.05 |
| GNAI2_S1PR5 | T_X2:T_X8:M_X4:M_X11 | M_X9 | 4111.35 |
| TGFB1_ENG | T_X2:T_X8:T_X9:M_X1:M_X5:M_X9 | M_X11 | 3334.96 |
| ENG_TGFB1 | M_X11 | T_X2:T_X8:T_X9:M_X1:M_X5:M_X9 | 3334.96 |
| IL16_CCR5 | M_X0:M_X2 | T_X2:M_X1:M_X6:M_X8 | 3176.54 |
| TNFSF9_TRAF2 | M_X4:M_X7:M_X11 | M_X7 | 2241.62 |
| ICAM2_ITGAM | M_X0 | M_X1:M_X6:M_X8 | 2107.78 |
| IL15_IL2RG | M_X1:M_X7 | T_X6:M_X0:M_X2 | 2089.32 |
| ICAM2_ITGB2 | M_X0 | T_X7:M_X1:M_X9 | 1775.12 |
| P4HB_GPR162 | M_X8 | M_X3 | 1774.24 |
| ICAM1_IL2RG | T_X2:T_X9:M_X1:M_X7 | T_X6:M_X0:M_X2 | 1640.19 |
| TGFB1_CXCR4 | T_X2:T_X8:T_X9:M_X1:M_X5:M_X9 | M_X9 | 1346.14 |
| TNFSF11_TNFRSF11A | T_X9 | M_X7 | 1225.30 |
| TNFRSF11A_TNFSF11 | M_X7 | T_X9 | 1225.30 |
| VEGFA_KDR | M_X8 | M_X11 | 1103.76 |
| KDR_VEGFA | M_X11 | M_X8 | 1103.76 |
| L1CAM_L1CAM | M_X11 | M_X11 | 1068.64 |
| ICAM2_ITGAL | M_X0 | M_X5:M_X9 | 907.61 |
| ADAM9_ITGB5 | M_X1 | M_X9 | 242.11 |
| SEMA4D_CD72 | M_X9 | M_X0:M_X3:M_X12 | 82.80 |
| CD72_SEMA4D | M_X0:M_X3:M_X12 | M_X9 | 82.80 |
| SEMA4D_PLXNB2 | M_X9 | M_X9 | 1.01 |
| PLXNB2_SEMA4D | M_X9 | M_X9 | 1.01 |

Table 2A-2B. Differentially Expressed Genes Associated with Time

TABLE 2A
Non-T cell clusters

| | Non- | Non- | Non- | Non- | Non- | Non- | Non- | Non- | Non- | Non- | Non- | Non- |
|---|---|---|---|---|---|---|---|---|---|---|---|---|
| Gene | T_0 | T_1 | T_10 | T_11 | T_2 | T_3 | T_4 | T_5 | T_6 | T_7 | T_8 | T_9 |
| KLRA7 | 0.00 | 0.00 | 0.00 | 0.00 | 0.00 | 0.00 | 0.00 | 0.00 | 0.00 | 0.00 | 0.00 | 0.00 |
| PENK | 0.00 | 0.00 | 0.00 | 0.00 | 0.00 | 0.00 | 0.00 | 0.00 | 0.00 | 0.00 | 0.00 | 0.00 |
| SPP1 | 0.00 | 0.00 | 0.00 | 0.00 | 0.00 | 0.00 | 1.62 | 0.00 | 0.00 | 0.00 | 0.00 | 9.70 |
| GM42418 | 3.10 | 3.83 | 1.84 | 1.60 | 5.20 | 5.28 | 3.71 | 1.85 | 2.54 | 2.03 | 1.38 | 3.10 |
| SAMD9L | 0.00 | 1.84 | 0.00 | 0.00 | 0.00 | 1.34 | 2.60 | 0.00 | 0.00 | 0.00 | 0.00 | 0.00 |
| ADRB2 | 0.00 | 2.01 | 0.00 | 0.00 | 0.00 | 2.61 | 0.00 | 0.00 | 2.32 | 0.00 | 0.00 | 0.00 |
| LGALS7 | 0.00 | 0.00 | 0.00 | 0.00 | 0.00 | 0.00 | 0.00 | 0.00 | 0.00 | 0.00 | 0.00 | 0.00 |
| SERPINE2 | 0.00 | 0.00 | 0.00 | 0.00 | 0.00 | 0.00 | 0.00 | 0.00 | 0.00 | 0.00 | 0.00 | 0.00 |
| TFF1 | 0.00 | 0.00 | 0.00 | 0.00 | 0.00 | 0.00 | 0.00 | 0.00 | 1.79 | 0.00 | 0.00 | 0.00 |
| CHKA | 0.00 | 0.00 | 0.00 | 0.00 | 0.00 | 0.00 | 0.00 | 0.00 | 0.00 | 0.00 | 0.00 | 0.00 |
| ATG7 | 0.00 | 0.00 | 0.00 | 0.00 | 0.00 | 0.00 | 0.00 | 0.00 | 0.00 | 0.00 | 0.00 | 0.00 |
| HIST1H2AP | 0.00 | 0.00 | 0.00 | 0.00 | 0.00 | 0.00 | 0.00 | 0.00 | 0.00 | 0.00 | 0.00 | 0.00 |
| S100A8 | 11.03 | 0.00 | 0.00 | 0.00 | 0.00 | 6.26 | 0.00 | 3.91 | 0.00 | 0.00 | 1.38 | 31.17 |
| IFIH1 | 0.00 | 1.86 | 0.00 | 0.00 | 0.00 | 0.00 | 1.50 | 0.00 | 0.00 | 0.00 | 0.00 | 0.00 |
| LGMN | 0.00 | 0.00 | 0.00 | 0.00 | 0.00 | 0.00 | 0.00 | 0.00 | 0.00 | 0.00 | 0.00 | 0.00 |
| MIR142HG | 2.55 | 0.00 | 0.00 | 0.00 | 0.00 | 0.00 | 0.00 | 0.00 | 0.00 | 0.00 | 0.00 | 0.00 |
| LILRB4A | 0.00 | 0.00 | 0.00 | 0.00 | 0.00 | 0.00 | 0.00 | 0.00 | 0.00 | 0.00 | 0.00 | 0.00 |
| AGAP2 | 0.00 | 0.00 | 0.00 | 0.00 | 0.00 | 0.00 | 0.00 | 0.00 | 0.00 | 0.00 | 0.00 | 0.00 |
| GM16586 | 0.00 | 0.00 | 0.00 | 0.00 | 0.00 | 0.00 | 0.00 | 0.00 | 0.00 | 0.00 | 0.00 | 0.00 |
| CCL8 | 0.00 | 0.00 | 0.00 | 0.00 | 0.00 | 0.00 | 0.00 | 0.00 | 0.00 | 0.00 | 0.00 | 0.00 |
| DCT | 0.00 | 0.00 | 0.00 | 0.00 | 0.00 | 0.00 | 0.00 | 0.00 | 0.00 | 0.00 | 0.00 | 0.00 |
| IFITM3 | 0.00 | 0.00 | 0.00 | 0.00 | 0.00 | 0.00 | 1.79 | 0.00 | 0.00 | 2.18 | 0.00 | 0.00 |
| C1QA | 0.00 | 2.10 | 0.00 | 0.00 | 0.00 | 0.00 | 0.00 | 0.00 | 4.92 | 0.00 | 0.00 | 0.00 |
| CTNND2 | 0.00 | 0.00 | 0.00 | 0.00 | 0.00 | 0.00 | 0.00 | 0.00 | 0.00 | 0.00 | 0.00 | 0.00 |
| PMS1 | 0.00 | 0.00 | 0.00 | 0.00 | 0.00 | 0.00 | 0.00 | 0.00 | 0.00 | 0.00 | 0.00 | 0.00 |
| TMEM209 | 0.00 | 0.00 | 0.00 | 0.00 | 0.00 | 0.00 | 0.00 | 0.00 | 0.00 | 0.00 | 0.00 | 0.00 |
| 1700025G04RIK | 0.00 | 0.00 | 0.00 | 0.00 | 0.00 | 0.00 | 0.00 | 0.00 | 0.00 | 0.00 | 0.00 | 0.00 |
| 5730455P16RIK | 0.00 | 0.00 | 0.00 | 0.00 | 0.00 | 0.00 | 1.35 | 0.00 | 0.00 | 0.00 | 0.00 | 0.00 |
| 9930111J21RIK2 | 0.00 | 0.00 | 0.00 | 0.00 | 0.00 | 0.00 | 0.00 | 0.00 | 1.36 | 0.00 | 0.00 | 0.00 |
| A130010J15RIK | 0.00 | 0.00 | 0.00 | 0.00 | 0.00 | 0.00 | 0.00 | 0.00 | 0.00 | 0.00 | 0.00 | 0.00 |
| A230046K03RIK | 0.00 | 0.00 | 0.00 | 0.00 | 0.00 | 0.00 | 0.00 | 0.00 | 0.00 | 0.00 | 0.00 | 0.00 |
| A330069E16RIK | 0.00 | 0.00 | 0.00 | 0.00 | 0.00 | 0.00 | 0.00 | 0.00 | 0.00 | 0.00 | 0.00 | 0.00 |
| A530040E14RIK | 0.00 | 1.73 | 0.00 | 0.00 | 0.00 | 0.00 | 2.58 | 0.00 | 0.00 | 0.00 | 0.00 | 0.00 |
| AA467197 | 0.00 | 0.00 | 0.00 | 0.00 | 0.00 | 0.00 | 0.00 | 0.00 | 0.00 | 0.00 | 0.00 | 2.62 |
| ABCC4 | 0.00 | 0.00 | 0.00 | 0.00 | 0.00 | 0.00 | 0.00 | 0.00 | 0.00 | 0.00 | 0.00 | 0.00 |
| AC125149.3 | 0.00 | 1.46 | 0.00 | 0.00 | 0.00 | 0.00 | 1.83 | 0.00 | 0.00 | 0.00 | 0.00 | 0.00 |
| ACADL | 0.00 | 0.00 | 0.00 | 0.00 | 0.00 | 0.00 | 1.51 | 0.00 | 0.00 | 0.00 | 0.00 | 0.00 |
| ACTB | 2.18 | 0.00 | 0.00 | 0.00 | 0.00 | 0.00 | 0.00 | 0.00 | 0.00 | 0.00 | 0.00 | 1.44 |
| ADAM8 | 0.00 | 0.00 | 0.00 | 0.00 | 0.00 | 0.00 | 0.00 | 0.00 | 0.00 | 0.00 | 0.00 | 2.01 |
| ADAP2 | 0.00 | 2.83 | 0.00 | 0.00 | 0.00 | 0.00 | 0.00 | 0.00 | 0.00 | 0.00 | 0.00 | 0.00 |
| ADAP2OS | 0.00 | 1.73 | 0.00 | 0.00 | 0.00 | 0.00 | 0.00 | 0.00 | 0.00 | 0.00 | 0.00 | 0.00 |
| ADPGK | 0.00 | 0.00 | 0.00 | 0.00 | 0.00 | 0.00 | 0.00 | 0.00 | 0.00 | 0.00 | 0.00 | 1.54 |
| AHCYL2 | 0.00 | 0.00 | 0.00 | 0.00 | 0.00 | 0.00 | 0.00 | 0.00 | 0.00 | 0.00 | 0.00 | 0.00 |
| AHNAK | 0.00 | 0.00 | 1.70 | 0.00 | 0.00 | 1.83 | 0.00 | 0.00 | 0.00 | 0.00 | 0.00 | 0.00 |
| AI607873 | 0.00 | 1.73 | 2.11 | 0.00 | 0.00 | 1.54 | 0.00 | 1.85 | 0.00 | 0.00 | 0.00 | 0.00 |
| AIF1 | 0.00 | 2.66 | 0.00 | 0.00 | 0.00 | 0.00 | 0.00 | 0.00 | 3.27 | 0.00 | 0.00 | 0.00 |
| AKAP2 | 0.00 | 0.00 | 0.00 | 0.00 | 0.00 | 0.00 | 0.00 | 0.00 | 2.32 | 0.00 | 0.00 | 0.00 |
| ANKRD33B | 0.00 | 0.00 | 0.00 | 0.00 | 0.00 | 0.00 | 0.00 | 0.00 | 0.00 | 0.00 | 0.00 | 3.78 |
| ANXA1 | 0.00 | 0.00 | 0.00 | 0.00 | 0.00 | 0.00 | 0.00 | 0.00 | 0.00 | 0.00 | 0.00 | 2.41 |
| APOD | 0.00 | 0.00 | 0.00 | 0.00 | 0.00 | 1.65 | 0.00 | 0.00 | 0.00 | 0.00 | 0.00 | 0.00 |
| APOE | 1.38 | 0.00 | 0.00 | 0.00 | 0.00 | 0.00 | 0.00 | 0.00 | 0.00 | 0.00 | 0.00 | 0.00 |
| APOL9B | 0.00 | 0.00 | 0.00 | 0.00 | 0.00 | 0.00 | 0.00 | 0.00 | 0.00 | 0.00 | 0.00 | 0.00 |
| AREG | 0.00 | 0.00 | 2.08 | 0.00 | 0.00 | 0.00 | 0.00 | 0.00 | 0.00 | 0.00 | 0.00 | 0.00 |
| ARG1 | 0.00 | 0.00 | 0.00 | 0.00 | 0.00 | 0.00 | 0.00 | 0.00 | 0.00 | 0.00 | 0.00 | 1.71 |
| ATAD2 | 0.00 | 0.00 | 0.00 | 0.00 | 0.00 | 0.00 | 0.00 | 0.00 | 0.00 | 0.00 | 0.00 | 0.00 |
| ATP2B1 | 0.00 | 0.00 | 0.00 | 0.00 | 0.00 | 0.00 | 0.00 | 0.00 | 0.00 | 0.00 | 0.00 | 0.00 |
| AVL9 | 0.00 | 0.00 | 0.00 | 0.00 | 0.00 | 0.00 | 0.00 | 0.00 | 0.00 | 0.00 | 0.00 | 0.00 |
| AY036118 | 2.79 | 3.45 | 5.44 | 5.37 | 3.43 | 7.00 | 1.64 | 6.49 | 4.27 | 3.47 | 1.38 | 0.00 |
| B3GALNT2 | 0.00 | 0.00 | 0.00 | 0.00 | 0.00 | 0.00 | 0.00 | 0.00 | 0.00 | 0.00 | 0.00 | 0.00 |
| BASP1 | 0.00 | 0.00 | 0.00 | 0.00 | 0.00 | 0.00 | 0.00 | 0.00 | 0.00 | 0.00 | 0.00 | 7.08 |
| BAZ1A | 0.00 | 0.00 | 0.00 | 0.00 | 0.00 | 0.00 | 0.00 | 0.00 | 0.00 | 0.00 | 0.00 | 0.00 |
| BC147527 | 0.00 | 1.42 | 0.00 | 0.00 | 0.00 | 0.00 | 0.00 | 0.00 | 0.00 | 0.00 | 0.00 | 0.00 |
| BCL11A | 0.00 | 0.00 | 0.00 | 0.00 | 0.00 | 0.00 | 0.00 | 0.00 | 0.00 | 0.00 | 0.00 | 0.00 |
| BCL2A1A | 0.00 | 0.00 | 0.00 | 0.00 | 0.00 | 0.00 | 1.91 | 0.00 | 0.00 | 0.00 | 0.00 | 0.00 |
| BCL2A1B | 1.93 | 0.00 | 0.00 | 0.00 | 0.00 | 0.00 | 0.00 | 0.00 | 0.00 | 0.00 | 0.00 | 0.00 |
| BHLHE40 | 0.00 | 0.00 | 0.00 | 0.00 | 0.00 | 0.00 | 0.00 | 0.00 | 0.00 | 0.00 | 0.00 | 0.00 |
| BNIP3 | 0.00 | 0.00 | 0.00 | 0.00 | 0.00 | 0.00 | 0.00 | 0.00 | 0.00 | 0.00 | 0.00 | 3.11 |
| BRPF1 | 0.00 | 0.00 | 0.00 | 0.00 | 0.00 | 0.00 | 0.00 | 0.00 | 0.00 | 0.00 | 0.00 | 0.00 |
| BST1 | 0.00 | 0.00 | 0.00 | 0.00 | 0.00 | 0.00 | 0.00 | 0.00 | 0.00 | 0.00 | 0.00 | 1.53 |
| BST2 | 0.00 | 1.80 | 0.00 | 0.00 | 0.00 | 0.00 | 0.00 | 0.00 | 1.54 | 0.00 | 0.00 | 0.00 |
| BTG3 | 0.00 | 0.00 | 0.00 | 0.00 | 0.00 | 0.00 | 0.00 | 0.00 | 0.00 | 0.00 | 0.00 | 0.00 |
| C130026I21RIK | 0.00 | 1.55 | 0.00 | 0.00 | 0.00 | 0.00 | 2.04 | 0.00 | 0.00 | 0.00 | 0.00 | 0.00 |
| C1QB | 0.00 | 2.69 | 0.00 | 0.00 | 0.00 | 0.00 | 0.00 | 0.00 | 4.92 | 0.00 | 0.00 | 0.00 |
| C1QC | 0.00 | 2.46 | 0.00 | 0.00 | 0.00 | 0.00 | 0.00 | 0.00 | 1.67 | 0.00 | 0.00 | 0.00 |
| C2CD4B | 0.00 | 0.00 | 0.00 | 0.00 | 0.00 | 0.00 | 0.00 | 0.00 | 0.00 | 4.60 | 0.00 | 0.00 |
| C3AR1 | 0.00 | 1.63 | 0.00 | 0.00 | 0.00 | 0.00 | 0.00 | 0.00 | 0.00 | 0.00 | 0.00 | 0.00 |
| CABLES1 | 0.00 | 0.00 | 0.00 | 0.00 | 0.00 | 0.00 | 1.37 | 0.00 | 0.00 | 0.00 | 0.00 | 0.00 |
| CACYBP | 2.47 | 0.00 | 0.00 | 0.00 | 0.00 | 0.00 | 0.00 | 0.00 | 0.00 | 0.00 | 0.00 | 0.00 |
| CALM1 | 0.00 | 0.00 | 2.13 | 0.00 | 0.00 | 0.00 | 0.00 | 0.00 | 0.00 | 0.00 | 0.00 | 0.00 |
| CARD19 | 0.00 | 0.00 | 0.00 | 0.00 | 0.00 | 0.00 | 0.00 | 0.00 | 0.00 | 0.00 | 0.00 | 6.03 |
| CCDC88C | 0.00 | 0.00 | 0.00 | 0.00 | 0.00 | 0.00 | 0.00 | 0.00 | 0.00 | 0.00 | 0.00 | 0.00 |
| CCL1 | 0.00 | 0.00 | 0.00 | 0.00 | 2.36 | 0.00 | 0.00 | 0.00 | 0.00 | 0.00 | 0.00 | 0.00 |
| CCL12 | 0.00 | 1.63 | 0.00 | 0.00 | 0.00 | 0.00 | 0.00 | 0.00 | 0.00 | 0.00 | 0.00 | 0.00 |
| CCL2 | 0.00 | 0.00 | 0.00 | 0.00 | 0.00 | 0.00 | 0.00 | 0.00 | 0.00 | 0.00 | 0.00 | 0.00 |
| CCL22 | 0.00 | 0.00 | 0.00 | 0.00 | 0.00 | 0.00 | 1.53 | 0.00 | 0.00 | 0.00 | 0.00 | 0.00 |
| CCL3 | 0.00 | 0.00 | 0.00 | 1.39 | 0.00 | 0.00 | 0.00 | 1.70 | 0.00 | 0.00 | 0.00 | 19.72 |
| CCL4 | 0.00 | 0.00 | 0.00 | 6.24 | 0.00 | 0.00 | 0.00 | 0.00 | 0.00 | 0.00 | 0.00 | 21.90 |
| CCL5 | 0.00 | 0.00 | 0.00 | 5.86 | 0.00 | 0.00 | 0.00 | 0.00 | 0.00 | 0.00 | 0.00 | 0.00 |
| CCL6 | 0.00 | 0.00 | 0.00 | 0.00 | 0.00 | 0.00 | 0.00 | 0.00 | 0.00 | 0.00 | 0.00 | 0.00 |
| CCL7 | 0.00 | 0.00 | 3.20 | 0.00 | 0.00 | 0.00 | 0.00 | 0.00 | 0.00 | 0.00 | 0.00 | 0.00 |
| CCNB1 | 0.00 | 0.00 | 0.00 | 0.00 | 0.00 | 0.00 | 1.32 | 0.00 | 0.00 | 0.00 | 0.00 | 0.00 |
| CCND1 | 0.00 | 1.85 | 0.00 | 0.00 | 0.00 | 0.00 | 0.00 | 0.00 | 0.00 | 0.00 | 0.00 | 0.00 |
| CCND2 | 0.00 | 0.00 | 0.00 | 0.00 | 0.00 | 0.00 | 2.58 | 0.00 | 0.00 | 0.00 | 0.00 | 0.00 |
| CCNL1 | 1.98 | 0.00 | 0.00 | 0.00 | 0.00 | 0.00 | 0.00 | 0.00 | 0.00 | 0.00 | 0.00 | 0.00 |
| CCNT2 | 0.00 | 1.58 | 0.00 | 0.00 | 0.00 | 0.00 | 0.00 | 0.00 | 0.00 | 0.00 | 0.00 | 0.00 |
| CCR1 | 0.00 | 0.00 | 0.00 | 0.00 | 0.00 | 0.00 | 0.00 | 0.00 | 0.00 | 0.00 | 0.00 | 8.91 |
| CCR2 | 0.00 | 0.00 | 0.00 | 0.00 | 0.00 | 0.00 | 0.00 | 2.98 | 0.00 | 0.00 | 0.00 | 0.00 |
| CCR3 | 0.00 | 0.00 | 0.00 | 0.00 | 0.00 | 0.00 | 0.00 | 0.00 | 0.00 | 0.00 | 0.00 | 0.00 |
| CCR5 | 0.00 | 0.00 | 0.00 | 0.00 | 0.00 | 0.00 | 1.95 | 0.00 | 0.00 | 0.00 | 0.00 | 0.00 |
| CCR9 | 0.00 | 0.00 | 0.00 | 0.00 | 0.00 | 0.00 | 0.00 | 0.00 | 0.00 | 0.00 | 0.00 | 0.00 |
| CCRL2 | 0.00 | 0.00 | 0.00 | 0.00 | 0.00 | 0.00 | 0.00 | 0.00 | 0.00 | 0.00 | 0.00 | 3.08 |
| CD14 | 0.00 | 0.00 | 0.00 | 1.31 | 0.00 | 0.00 | 0.00 | 0.00 | 0.00 | 0.00 | 0.00 | 6.94 |
| CD200 | 0.00 | 0.00 | 0.00 | 0.00 | 0.00 | 0.00 | 0.00 | 0.00 | 0.00 | 0.00 | 0.00 | 0.00 |
| CD209D | 0.00 | 0.00 | 0.00 | 0.00 | 0.00 | 0.00 | 0.00 | 0.00 | 0.00 | 0.00 | 0.00 | 0.00 |
| CD24A | 0.00 | 3.16 | 0.00 | 0.00 | 0.00 | 0.00 | 0.00 | 0.00 | 2.94 | 0.00 | 0.00 | 9.81 |
| CD28 | 0.00 | 0.00 | 0.00 | 0.00 | 1.34 | 0.00 | 0.00 | 4.80 | 0.00 | 0.00 | 0.00 | 0.00 |
| CD2AP | 0.00 | 0.00 | 1.48 | 0.00 | 0.00 | 0.00 | 0.00 | 0.00 | 0.00 | 0.00 | 0.00 | 0.00 |
| CD300C | 0.00 | 0.00 | 0.00 | 0.00 | 0.00 | 0.00 | 1.85 | 0.00 | 0.00 | 0.00 | 0.00 | 0.00 |
| CD302 | 0.00 | 0.00 | 0.00 | 0.00 | 0.00 | 0.00 | 0.00 | 0.00 | 0.00 | 0.00 | 0.00 | 0.00 |
| CD4 | 0.00 | 0.00 | 0.00 | 0.00 | 0.00 | 0.00 | 0.00 | 0.00 | 0.00 | 0.00 | 0.00 | 0.00 |
| CD40 | 0.00 | 1.42 | 0.00 | 0.00 | 0.00 | 0.00 | 2.21 | 0.00 | 0.00 | 0.00 | 0.00 | 0.00 |
| CD52 | 0.00 | 0.00 | 0.00 | 0.00 | 0.00 | 0.00 | 1.37 | 0.00 | 0.00 | 0.00 | 0.00 | 0.00 |
| CD63 | 0.00 | 0.00 | 0.00 | 0.00 | 0.00 | 0.00 | 0.00 | 0.00 | 0.00 | 0.00 | 0.00 | 3.10 |
| CD69 | 0.00 | 0.00 | 0.00 | 0.00 | 0.00 | 0.00 | 0.00 | 0.00 | 0.00 | 0.00 | 0.00 | 1.63 |
| CD72 | 0.00 | 2.75 | 0.00 | 0.00 | 0.00 | 0.00 | 0.00 | 0.00 | 0.00 | 0.00 | 0.00 | 0.00 |
| CD74 | 0.00 | 0.00 | 0.00 | 0.00 | 1.88 | 0.00 | 0.00 | 0.00 | 0.00 | 0.00 | 0.00 | 0.00 |
| CD79A | 0.00 | 0.00 | 0.00 | 0.00 | 0.00 | 0.00 | 0.00 | 0.00 | 0.00 | 0.00 | 0.00 | 1.42 |
| CD83 | 11.65 | 0.00 | 0.00 | 0.00 | 0.00 | 0.00 | 0.00 | 0.00 | 0.00 | 0.00 | 0.00 | 0.00 |
| CD8A | 0.00 | 0.00 | 0.00 | 0.00 | 0.00 | 0.00 | 0.00 | 0.00 | 0.00 | 0.00 | 0.00 | 0.00 |
| CD9 | 0.00 | 3.83 | 0.00 | 0.00 | 0.00 | 0.00 | 0.00 | 0.00 | 1.59 | 0.00 | 0.00 | 2.53 |
| CDK13 | 0.00 | 0.00 | 0.00 | 0.00 | 0.00 | 0.00 | 0.00 | 0.00 | 0.00 | 0.00 | 0.00 | 0.00 |
| CDK20 | 1.66 | 0.00 | 0.00 | 0.00 | 0.00 | 0.00 | 0.00 | 0.00 | 0.00 | 0.00 | 0.00 | 0.00 |
| CDKN2C | 0.00 | 0.00 | 0.00 | 0.00 | 0.00 | 0.00 | 2.04 | 0.00 | 0.00 | 0.00 | 0.00 | 0.00 |
| CEBPB | 2.98 | 0.00 | 0.00 | 0.00 | 0.00 | 0.00 | 0.00 | 0.00 | 0.00 | 0.00 | 0.00 | 0.00 |
| CEP170 | 0.00 | 0.00 | 0.00 | 0.00 | 0.00 | 0.00 | 0.00 | 0.00 | 0.00 | 0.00 | 0.00 | 0.00 |
| CEP250 | 0.00 | 0.00 | 0.00 | 0.00 | 0.00 | 0.00 | 0.00 | 0.00 | 0.00 | 0.00 | 0.00 | 0.00 |
| CFB | 0.00 | 3.16 | 0.00 | 0.00 | 0.00 | 0.00 | 0.00 | 0.00 | 4.92 | 0.00 | 0.00 | 0.00 |
| CITED2 | 0.00 | 0.00 | 0.00 | 0.00 | 0.00 | 0.00 | 0.00 | 0.00 | 0.00 | 0.00 | 0.00 | 0.00 |
| CKAP4 | 0.00 | 0.00 | 0.00 | 0.00 | 0.00 | 0.00 | 0.00 | 0.00 | 0.00 | 0.00 | 0.00 | 0.00 |
| CLEC4D | 0.00 | 0.00 | 0.00 | 0.00 | 0.00 | 0.00 | 0.00 | 0.00 | 0.00 | 0.00 | 0.00 | 5.75 |
| CLEC4E | 0.00 | 0.00 | 0.00 | 0.00 | 0.00 | 0.00 | 0.00 | 0.00 | 0.00 | 0.00 | 0.00 | 2.66 |
| CLEC4N | 0.00 | 0.00 | 0.00 | 0.00 | 0.00 | 0.00 | 0.00 | 0.00 | 0.00 | 0.00 | 0.00 | 2.92 |
| CLK1 | 0.00 | 0.00 | 0.00 | 0.00 | 0.00 | 0.00 | 0.00 | 0.00 | 0.00 | 0.00 | 0.00 | 0.00 |
| CMPK2 | 0.00 | 1.35 | 0.00 | 0.00 | 0.00 | 0.00 | 0.00 | 0.00 | 0.00 | 0.00 | 0.00 | 0.00 |
| COL4A3BP | 0.00 | 0.00 | 0.00 | 0.00 | 0.00 | 0.00 | 0.00 | 0.00 | 0.00 | 0.00 | 0.00 | 0.00 |
| COX6A2 | 0.00 | 0.00 | 0.00 | 0.00 | 0.00 | 0.00 | 0.00 | 0.00 | 0.00 | 0.00 | 0.00 | 0.00 |
| CREM | 4.87 | 0.00 | 2.08 | 0.00 | 0.00 | 0.00 | 0.00 | 0.00 | 0.00 | 0.00 | 0.00 | 0.00 |
| CROT | 0.00 | 0.00 | 0.00 | 0.00 | 0.00 | 0.00 | 0.00 | 0.00 | 0.00 | 0.00 | 0.00 | 0.00 |
| CSF1 | 0.00 | 0.00 | 0.00 | 0.00 | 0.00 | 0.00 | 0.00 | 0.00 | 0.00 | 0.00 | 0.00 | 1.59 |
| CSPRS | 0.00 | 1.42 | 0.00 | 0.00 | 0.00 | 0.00 | 0.00 | 0.00 | 0.00 | 0.00 | 0.00 | 0.00 |
| CSRNP1 | 0.00 | 0.00 | 0.00 | 0.00 | 0.00 | 0.00 | 0.00 | 0.00 | 0.00 | 0.00 | 0.00 | 4.32 |
| CSTB | 0.00 | 0.00 | 0.00 | 0.00 | 0.00 | 0.00 | 0.00 | 0.00 | 0.00 | 0.00 | 0.00 | 4.25 |
| CTLA4 | 0.00 | 0.00 | 0.00 | 0.00 | 0.00 | 0.00 | 0.00 | 1.70 | 0.00 | 0.00 | 0.00 | 0.00 |
| CTSD | 0.00 | 0.00 | 0.00 | 0.00 | 0.00 | 0.00 | 0.00 | 0.00 | 0.00 | 0.00 | 0.00 | 1.80 |
| CTSS | 0.00 | 1.77 | 0.00 | 0.00 | 0.00 | 0.00 | 1.92 | 0.00 | 1.54 | 0.00 | 0.00 | 0.00 |
| CXCL10 | 0.00 | 0.00 | 0.00 | 0.00 | 0.00 | 0.00 | 1.35 | 0.00 | 0.00 | 0.00 | 0.00 | 0.00 |
| CXCL2 | 0.00 | 1.40 | 0.00 | 0.00 | 0.00 | 5.76 | 0.00 | 1.30 | 0.00 | 0.00 | 0.00 | 44.38 |
| CXCL9 | 0.00 | 0.00 | 0.00 | 1.50 | 0.00 | 0.00 | 0.00 | 0.00 | 0.00 | 0.00 | 0.00 | 0.00 |
| CXCR4 | 0.00 | 0.00 | 0.00 | 0.00 | 0.00 | 0.00 | 0.00 | 0.00 | 0.00 | 0.00 | 0.00 | 3.95 |
| CXCR6 | 0.00 | 0.00 | 1.85 | 0.00 | 0.00 | 0.00 | 0.00 | 0.00 | 0.00 | 0.00 | 0.00 | 0.00 |
| CYBB | 0.00 | 0.00 | 0.00 | 0.00 | 0.00 | 0.00 | 0.00 | 0.00 | 0.00 | 0.00 | 0.00 | 0.00 |
| CYFIP1 | 0.00 | 0.00 | 0.00 | 0.00 | 0.00 | 0.00 | 0.00 | 0.00 | 2.12 | 0.00 | 0.00 | 0.00 |
| D13ERTD608E | 0.00 | 0.00 | 0.00 | 0.00 | 0.00 | 0.00 | 0.00 | 0.00 | 0.00 | 0.00 | 0.00 | 0.00 |
| DAB2 | 1.43 | 0.00 | 0.00 | 0.00 | 0.00 | 0.00 | 0.00 | 0.00 | 0.00 | 0.00 | 0.00 | 0.00 |
| DDIT3 | 0.00 | 0.00 | 0.00 | 0.00 | 0.00 | 0.00 | 0.00 | 0.00 | 0.00 | 0.00 | 0.00 | 1.41 |
| DDX5 | 0.00 | 0.00 | 0.00 | 0.00 | 0.00 | 0.00 | 0.00 | 0.00 | 0.00 | 0.00 | 0.00 | 0.00 |
| DDX58 | 0.00 | 1.85 | 0.00 | 0.00 | 0.00 | 0.00 | 0.00 | 0.00 | 0.00 | 0.00 | 0.00 | 0.00 |
| DDX60 | 0.00 | 2.11 | 0.00 | 0.00 | 0.00 | 0.00 | 1.36 | 0.00 | 0.00 | 0.00 | 0.00 | 0.00 |
| DGAT2 | 0.00 | 0.00 | 0.00 | 0.00 | 0.00 | 0.00 | 0.00 | 0.00 | 0.00 | 0.00 | 0.00 | 0.00 |
| DGCR8 | 0.00 | 0.00 | 0.00 | 0.00 | 0.00 | 0.00 | 0.00 | 0.00 | 0.00 | 0.00 | 0.00 | 0.00 |
| DGKG | 0.00 | 0.00 | 0.00 | 0.00 | 0.00 | 0.00 | 0.00 | 0.00 | 0.00 | 0.00 | 0.00 | 0.00 |
| DHX29 | 0.00 | 0.00 | 0.00 | 0.00 | 0.00 | 0.00 | 0.00 | 0.00 | 0.00 | 0.00 | 0.00 | 0.00 |
| DHX58 | 0.00 | 2.16 | 0.00 | 0.00 | 0.00 | 0.00 | 0.00 | 0.00 | 0.00 | 0.00 | 0.00 | 0.00 |
| DIP2B | 0.00 | 0.00 | 0.00 | 0.00 | 0.00 | 0.00 | 0.00 | 0.00 | 0.00 | 0.00 | 0.00 | 0.00 |
| DMKN | 0.00 | 0.00 | 0.00 | 0.00 | 0.00 | 0.00 | 0.00 | 0.00 | 0.00 | 0.00 | 0.00 | 2.49 |
| DNAJA1 | 1.91 | 0.00 | 0.00 | 0.00 | 0.00 | 0.00 | 0.00 | 0.00 | 0.00 | 0.00 | 0.00 | 1.37 |
| DNAJB1 | 18.84 | 0.00 | 0.00 | 0.00 | 1.74 | 0.00 | 0.00 | 0.00 | 2.52 | 0.00 | 0.00 | 0.00 |
| DNAJB4 | 0.00 | 0.00 | 0.00 | 0.00 | 0.00 | 0.00 | 0.00 | 0.00 | 0.00 | 0.00 | 0.00 | 0.00 |
| DNAJB9 | 1.71 | 0.00 | 0.00 | 0.00 | 0.00 | 0.00 | 0.00 | 0.00 | 0.00 | 0.00 | 0.00 | 0.00 |
| DNTTIP1 | 0.00 | 0.00 | 0.00 | 0.00 | 0.00 | 0.00 | 0.00 | 0.00 | 0.00 | 0.00 | 0.00 | 0.00 |
| DPYSL2 | 0.00 | 1.53 | 0.00 | 0.00 | 0.00 | 0.00 | 0.00 | 0.00 | 0.00 | 0.00 | 0.00 | 0.00 |
| DUSP1 | 2.35 | 0.00 | 0.00 | 1.67 | 0.00 | 0.00 | 0.00 | 0.00 | 0.00 | 0.00 | 0.00 | 2.16 |
| DUSP2 | 0.00 | 0.00 | 0.00 | 0.00 | 0.00 | 0.00 | 0.00 | 0.00 | 0.00 | 0.00 | 0.00 | 0.00 |
| EBF1 | 0.00 | 0.00 | 0.00 | 0.00 | 0.00 | 0.00 | 0.00 | 0.00 | 0.00 | 0.00 | 0.00 | 3.35 |
| EGLN3 | 0.00 | 0.00 | 0.00 | 0.00 | 0.00 | 0.00 | 0.00 | 0.00 | 0.00 | 0.00 | 0.00 | 1.84 |
| EGR1 | 0.00 | 0.00 | 0.00 | 0.00 | 0.00 | 0.00 | 0.00 | 0.00 | 0.00 | 0.00 | 0.00 | 4.87 |
| EIF5 | 0.00 | 0.00 | 0.00 | 0.00 | 0.00 | 0.00 | 0.00 | 0.00 | 0.00 | 0.00 | 0.00 | 2.12 |
| EML4 | 0.00 | 0.00 | 2.11 | 0.00 | 0.00 | 0.00 | 0.00 | 0.00 | 0.00 | 0.00 | 0.00 | 0.00 |
| ENPP2 | 0.00 | 0.00 | 0.00 | 0.00 | 0.00 | 0.00 | 0.00 | 0.00 | 0.00 | 0.00 | 0.00 | 0.00 |
| EOMES | 0.00 | 0.00 | 0.00 | 0.00 | 0.00 | 0.00 | 0.00 | 0.00 | 0.00 | 0.00 | 0.00 | 0.00 |
| ERDR1 | 2.85 | 0.00 | 0.00 | 0.00 | 0.00 | 2.07 | 0.00 | 0.00 | 2.04 | 0.00 | 0.00 | 0.00 |
| ERGIC2 | 0.00 | 0.00 | 0.00 | 0.00 | 0.00 | 0.00 | 0.00 | 0.00 | 0.00 | 0.00 | 0.00 | 1.41 |
| ERO1L | 0.00 | 0.00 | 0.00 | 0.00 | 0.00 | 0.00 | 0.00 | 0.00 | 0.00 | 0.00 | 0.00 | 5.45 |
| ETNK1 | 0.00 | 1.98 | 0.00 | 0.00 | 0.00 | 0.00 | 0.00 | 0.00 | 0.00 | 0.00 | 0.00 | 0.00 |
| ETS2 | 0.00 | 0.00 | 0.00 | 0.00 | 0.00 | 0.00 | 0.00 | 0.00 | 0.00 | 0.00 | 0.00 | 2.79 |
| ETV3 | 0.00 | 0.00 | 0.00 | 0.00 | 0.00 | 0.00 | 3.80 | 0.00 | 0.00 | 0.00 | 0.00 | 0.00 |
| FAM162A | 0.00 | 0.00 | 0.00 | 0.00 | 0.00 | 0.00 | 0.00 | 0.00 | 0.00 | 0.00 | 0.00 | 1.49 |
| FAM178A | 0.00 | 0.00 | 0.00 | 0.00 | 0.00 | 0.00 | 1.47 | 0.00 | 0.00 | 0.00 | 0.00 | 0.00 |
| FAM46A | 0.00 | 0.00 | 0.00 | 0.00 | 0.00 | 3.19 | 0.00 | 0.00 | 0.00 | 0.00 | 0.00 | 0.00 |
| FAM71A | 0.00 | 0.00 | 0.00 | 0.00 | 0.00 | 0.00 | 0.00 | 2.62 | 0.00 | 0.00 | 0.00 | 0.00 |
| FAM76B | 0.00 | 0.00 | 0.00 | 0.00 | 0.00 | 0.00 | 0.00 | 0.00 | 2.29 | 0.00 | 0.00 | 0.00 |
| FBLN5 | 0.00 | 0.00 | 0.00 | 0.00 | 0.00 | 0.00 | 0.00 | 0.00 | 1.51 | 0.00 | 0.00 | 0.00 |
| FBXW9 | 0.00 | 0.00 | 0.00 | 0.00 | 0.00 | 0.00 | 0.00 | 0.00 | 0.00 | 0.00 | 0.00 | 0.00 |
| FCER1G | 0.00 | 0.00 | 0.00 | 0.00 | 0.00 | 0.00 | 0.00 | 0.00 | 0.00 | 0.00 | 0.00 | 0.00 |
| FCGR1 | 0.00 | 2.34 | 0.00 | 0.00 | 0.00 | 0.00 | 3.74 | 0.00 | 0.00 | 0.00 | 0.00 | 0.00 |
| FCGR3 | 0.00 | 0.00 | 0.00 | 0.00 | 0.00 | 0.00 | 0.00 | 0.00 | 0.00 | 0.00 | 0.00 | 0.00 |
| FDFT1 | 0.00 | 0.00 | 0.00 | 0.00 | 0.00 | 0.00 | 0.00 | 0.00 | 0.00 | 0.00 | 0.00 | 1.32 |
| FILIP1L | 0.00 | 1.65 | 0.00 | 0.00 | 0.00 | 0.00 | 0.00 | 0.00 | 0.00 | 0.00 | 0.00 | 0.00 |
| FNDC3A | 0.00 | 1.73 | 0.00 | 0.00 | 0.00 | 0.00 | 0.00 | 0.00 | 0.00 | 0.00 | 0.00 | 0.00 |
| FOSB | 3.10 | 0.00 | 0.00 | 0.00 | 0.00 | 0.00 | 0.00 | 0.00 | 0.00 | 0.00 | 0.00 | 0.00 |
| FRMD4A | 0.00 | 2.01 | 0.00 | 0.00 | 0.00 | 0.00 | 0.00 | 0.00 | 0.00 | 0.00 | 0.00 | 0.00 |
| FRMD4B | 0.00 | 0.00 | 0.00 | 0.00 | 0.00 | 0.00 | 0.00 | 0.00 | 0.00 | 0.00 | 0.00 | 0.00 |
| FSCN1 | 0.00 | 0.00 | 3.87 | 0.00 | 0.00 | 0.00 | 0.00 | 0.00 | 0.00 | 0.00 | 0.00 | 0.00 |
| FTH1 | 0.00 | 0.00 | 0.00 | 0.00 | 0.00 | 0.00 | 0.00 | 0.00 | 0.00 | 0.00 | 0.00 | 6.05 |
| FTL1 | 1.40 | 0.00 | 0.00 | 0.00 | 0.00 | 0.00 | 0.00 | 0.00 | 0.00 | 0.00 | 0.00 | 0.00 |
| G0S2 | 0.00 | 0.00 | 0.00 | 0.00 | 0.00 | 0.00 | 0.00 | 0.00 | 0.00 | 0.00 | 1.67 | 16.62 |
| GADD45B | 1.73 | 0.00 | 0.00 | 0.00 | 0.00 | 0.00 | 0.00 | 0.00 | 0.00 | 0.00 | 0.00 | 2.73 |
| GADD45G | 6.48 | 0.00 | 0.00 | 0.00 | 0.00 | 0.00 | 0.00 | 0.00 | 0.00 | 0.00 | 0.00 | 0.00 |
| GAS2 | 0.00 | 0.00 | 0.00 | 0.00 | 0.00 | 0.00 | 0.00 | 0.00 | 0.00 | 0.00 | 0.00 | 0.00 |
| GBP3 | 0.00 | 0.00 | 0.00 | 0.00 | 0.00 | 0.00 | 0.00 | 0.00 | 0.00 | 0.00 | 0.00 | 0.00 |
| GBP4 | 0.00 | 1.37 | 0.00 | 0.00 | 0.00 | 0.00 | 0.00 | 0.00 | 0.00 | 0.00 | 0.00 | 0.00 |
| GBP5 | 0.00 | 0.00 | 0.00 | 0.00 | 0.00 | 0.00 | 1.82 | 0.00 | 0.00 | 0.00 | 0.00 | 0.00 |
| GBP9 | 0.00 | 0.00 | 0.00 | 0.00 | 0.00 | 0.00 | 0.00 | 0.00 | 0.00 | 0.00 | 0.00 | 0.00 |
| GCNT2 | 0.00 | 0.00 | 0.00 | 0.00 | 0.00 | 0.00 | 0.00 | 0.00 | 1.40 | 0.00 | 0.00 | 0.00 |
| GDPGP1 | 0.00 | 0.00 | 0.00 | 0.00 | 0.00 | 0.00 | 0.00 | 0.00 | 0.00 | 0.00 | 0.00 | 0.00 |
| GEM | 3.06 | 0.00 | 0.00 | 0.00 | 0.00 | 0.00 | 0.00 | 0.00 | 0.00 | 0.00 | 0.00 | 0.00 |
| GGA3 | 0.00 | 0.00 | 0.00 | 0.00 | 0.00 | 0.00 | 0.00 | 0.00 | 0.00 | 0.00 | 0.00 | 0.00 |
| GIMAP9 | 0.00 | 0.00 | 0.00 | 0.00 | 0.00 | 0.00 | 0.00 | 0.00 | 0.00 | 0.00 | 0.00 | 0.00 |
| GINS2 | 0.00 | 0.00 | 1.38 | 0.00 | 0.00 | 0.00 | 0.00 | 0.00 | 0.00 | 0.00 | 0.00 | 0.00 |
| GM10076 | 4.44 | 0.00 | 0.00 | 0.00 | 0.00 | 2.94 | 0.00 | 1.70 | 0.00 | 0.00 | 0.00 | 0.00 |
| GM10116 | 0.00 | 0.00 | 0.00 | 0.00 | 0.00 | 0.00 | 0.00 | 0.00 | 0.00 | 0.00 | 0.00 | 1.40 |
| GM10263 | 0.00 | 0.00 | 0.00 | 0.00 | 0.00 | 0.00 | 0.00 | 0.00 | 0.00 | 0.00 | 0.00 | 0.00 |
| GM10269 | 0.00 | 0.00 | 0.00 | 0.00 | 0.00 | 0.00 | 0.00 | 0.00 | 0.00 | 0.00 | 0.00 | 0.00 |
| GM11175 | 0.00 | 0.00 | 0.00 | 0.00 | 0.00 | 0.00 | 0.00 | 0.00 | 1.70 | 0.00 | 0.00 | 0.00 |
| GM11808 | 0.00 | 0.00 | 0.00 | 0.00 | 0.00 | 0.00 | 0.00 | 0.00 | 0.00 | 0.00 | 0.00 | 0.00 |
| GM12185 | 0.00 | 0.00 | 0.00 | 0.00 | 0.00 | 0.00 | 0.00 | 0.00 | 0.00 | 0.00 | 0.00 | 0.00 |
| GM12216 | 0.00 | 0.00 | 0.00 | 4.62 | 0.00 | 3.84 | 0.00 | 0.00 | 0.00 | 0.00 | 0.00 | 0.00 |
| GM17056 | 1.83 | 0.00 | 0.00 | 0.00 | 0.00 | 0.00 | 0.00 | 0.00 | 0.00 | 0.00 | 0.00 | 2.31 |
| GM2000 | 2.85 | 0.00 | 0.00 | 0.00 | 0.00 | 3.24 | 1.39 | 0.00 | 0.00 | 0.00 | 0.00 | 0.00 |
| GM26518 | 0.00 | 0.00 | 0.00 | 0.00 | 0.00 | 0.00 | 0.00 | 0.00 | 0.00 | 0.00 | 0.00 | 0.00 |
| GM26522 | 0.00 | 0.00 | 0.00 | 0.00 | 0.00 | 0.00 | 1.31 | 0.00 | 0.00 | 0.00 | 0.00 | 0.00 |
| GM26532 | 0.00 | 1.66 | 0.00 | 0.00 | 0.00 | 0.00 | 0.00 | 1.30 | 1.54 | 0.00 | 0.00 | 0.00 |
| GM26541 | 0.00 | 0.00 | 0.00 | 0.00 | 0.00 | 0.00 | 0.00 | 0.00 | 0.00 | 0.00 | 0.00 | 0.00 |
| GM26545 | 0.00 | 0.00 | 0.00 | 0.00 | 0.00 | 0.00 | 0.00 | 0.00 | 0.00 | 0.00 | 0.00 | 0.00 |
| GM26699 | 0.00 | 0.00 | 0.00 | 0.00 | 0.00 | 0.00 | 0.00 | 0.00 | 0.00 | 0.00 | 0.00 | 0.00 |
| GM26917 | 0.00 | 0.00 | 0.00 | 3.58 | 0.00 | 0.00 | 1.52 | 0.00 | 0.00 | 0.00 | 0.00 | 0.00 |
| GM4070 | 0.00 | 0.00 | 0.00 | 0.00 | 0.00 | 0.00 | 0.00 | 0.00 | 0.00 | 0.00 | 0.00 | 0.00 |
| GM43603 | 0.00 | 0.00 | 0.00 | 0.00 | 0.00 | 0.00 | 0.00 | 0.00 | 0.00 | 0.00 | 0.00 | 0.00 |
| GM4955 | 0.00 | 3.45 | 0.00 | 0.00 | 0.00 | 0.00 | 2.21 | 0.00 | 1.70 | 0.00 | 0.00 | 0.00 |
| GM5093 | 0.00 | 0.00 | 0.00 | 0.00 | 0.00 | 0.00 | 0.00 | 0.00 | 0.00 | 0.00 | 0.00 | 0.00 |
| GM8186 | 0.00 | 0.00 | 0.00 | 0.00 | 0.00 | 0.00 | 0.00 | 0.00 | 0.00 | 0.00 | 0.00 | 0.00 |
| GM8797 | 1.63 | 0.00 | 0.00 | 0.00 | 0.00 | 0.00 | 0.00 | 0.00 | 0.00 | 0.00 | 0.00 | 0.00 |
| GM8953 | 0.00 | 1.79 | 0.00 | 0.00 | 0.00 | 0.00 | 0.00 | 0.00 | 0.00 | 0.00 | 0.00 | 0.00 |
| GPD2 | 0.00 | 0.00 | 0.00 | 0.00 | 0.00 | 0.00 | 1.66 | 0.00 | 0.00 | 0.00 | 0.00 | 0.00 |
| GPNMB | 0.00 | 2.47 | 0.00 | 0.00 | 0.00 | 1.52 | 0.00 | 0.00 | 0.00 | 0.00 | 0.00 | 0.00 |
| GRB2 | 0.00 | 0.00 | 0.00 | 0.00 | 0.00 | 0.00 | 1.50 | 0.00 | 0.00 | 0.00 | 0.00 | 0.00 |
| GRINA | 0.00 | 0.00 | 0.00 | 0.00 | 0.00 | 0.00 | 0.00 | 0.00 | 0.00 | 0.00 | 0.00 | 2.42 |
| GSR | 0.00 | 0.00 | 0.00 | 0.00 | 0.00 | 0.00 | 0.00 | 0.00 | 2.22 | 0.00 | 0.00 | 0.00 |
| GZMA | 0.00 | 0.00 | 0.00 | 0.00 | 0.00 | 0.00 | 0.00 | 0.00 | 0.00 | 0.00 | 0.00 | 0.00 |
| GZMB | 0.00 | 0.00 | 1.59 | 0.00 | 0.00 | 0.00 | 0.00 | 0.00 | 0.00 | 0.00 | 0.00 | 0.00 |
| H2-AA | 0.00 | 1.73 | 0.00 | 0.00 | 1.44 | 0.00 | 0.00 | 0.00 | 1.38 | 0.00 | 1.67 | 0.00 |
| H2-AB1 | 0.00 | 0.00 | 0.00 | 0.00 | 0.00 | 0.00 | 0.00 | 0.00 | 0.00 | 0.00 | 1.38 | 0.00 |
| H2-EB1 | 0.00 | 1.55 | 0.00 | 0.00 | 0.00 | 0.00 | 0.00 | 0.00 | 0.00 | 0.00 | 1.67 | 2.12 |
| H2-M3 | 0.00 | 1.37 | 0.00 | 0.00 | 0.00 | 0.00 | 0.00 | 0.00 | 0.00 | 0.00 | 0.00 | 0.00 |
| HBA-A1 | 0.00 | 0.00 | 0.00 | 0.00 | 0.00 | 0.00 | 0.00 | 0.00 | 0.00 | 0.00 | 0.00 | 37.16 |
| HBA-A2 | 0.00 | 0.00 | 0.00 | 0.00 | 0.00 | 0.00 | 0.00 | 0.00 | 0.00 | 0.00 | 0.00 | 29.41 |
| HBB-BS | 0.00 | 0.00 | 0.00 | 0.00 | 0.00 | 0.00 | 0.00 | 0.00 | 0.00 | 0.00 | 0.00 | 18.59 |
| HBB-BT | 0.00 | 0.00 | 0.00 | 0.00 | 0.00 | 0.00 | 0.00 | 0.00 | 0.00 | 0.00 | 0.00 | 0.00 |
| HCAR2 | 0.00 | 0.00 | 0.00 | 0.00 | 0.00 | 0.00 | 0.00 | 0.00 | 0.00 | 0.00 | 0.00 | 8.40 |
| HDC | 0.00 | 0.00 | 0.00 | 0.00 | 0.00 | 0.00 | 0.00 | 0.00 | 0.00 | 0.00 | 0.00 | 13.51 |
| HDLBP | 0.00 | 0.00 | 0.00 | 0.00 | 0.00 | 0.00 | 0.00 | 0.00 | 0.00 | 0.00 | 0.00 | 0.00 |
| HERC6 | 0.00 | 0.00 | 0.00 | 0.00 | 0.00 | 0.00 | 2.12 | 0.00 | 1.79 | 0.00 | 0.00 | 0.00 |
| HES1 | 0.00 | 0.00 | 0.00 | 0.00 | 0.00 | 0.00 | 0.00 | 0.00 | 0.00 | 0.00 | 0.00 | 0.00 |
| HILPDA | 0.00 | 0.00 | 0.00 | 0.00 | 0.00 | 0.00 | 0.00 | 0.00 | 0.00 | 0.00 | 0.00 | 12.38 |
| HIST1H1B | 0.00 | 0.00 | 0.00 | 0.00 | 0.00 | 0.00 | 0.00 | 0.00 | 0.00 | 0.00 | 0.00 | 0.00 |
| HIST1H1C | 0.00 | 0.00 | 0.00 | 0.00 | 0.00 | 0.00 | 0.00 | 0.00 | 0.00 | 0.00 | 0.00 | 0.00 |
| HIST1H1E | 0.00 | 0.00 | 0.00 | 0.00 | 1.44 | 0.00 | 0.00 | 0.00 | 0.00 | 0.00 | 0.00 | 0.00 |
| HIST1H2BC | 0.00 | 0.00 | 0.00 | 0.00 | 0.00 | 0.00 | 0.00 | 0.00 | 0.00 | 0.00 | 0.00 | 4.62 |
| HIST1H2BJ | 0.00 | 0.00 | 0.00 | 0.00 | 0.00 | 0.00 | 0.00 | 0.00 | 0.00 | 0.00 | 0.00 | 0.00 |
| HIST1H4H | 0.00 | 0.00 | 0.00 | 0.00 | 0.00 | 0.00 | 0.00 | 0.00 | 0.00 | 0.00 | 0.00 | 0.00 |
| HIST2H2AA1 | 0.00 | 0.00 | 0.00 | 0.00 | 0.00 | 0.00 | 0.00 | 0.00 | 0.00 | 0.00 | 0.00 | 1.62 |
| HMOX1 | 0.00 | 0.00 | 0.00 | 0.00 | 0.00 | 0.00 | 0.00 | 0.00 | 0.00 | 0.00 | 0.00 | 4.03 |
| HPGDS | 0.00 | 0.00 | 0.00 | 0.00 | 0.00 | 0.00 | 0.00 | 0.00 | 0.00 | 0.00 | 0.00 | 0.00 |
| HSP90AA1 | 4.78 | 0.00 | 0.00 | 0.00 | 0.00 | 0.00 | 0.00 | 0.00 | 0.00 | 0.00 | 0.00 | 0.00 |
| HSP90AB1 | 4.44 | 0.00 | 0.00 | 0.00 | 0.00 | 0.00 | 0.00 | 0.00 | 0.00 | 0.00 | 0.00 | 0.00 |
| HSPA1A | 53.05 | 0.00 | 1.47 | 11.97 | 0.00 | 0.00 | 0.00 | 0.00 | 0.00 | 4.91 | 0.00 | 4.14 |
| HSPA1B | 8.63 | 0.00 | 0.00 | 0.00 | 0.00 | 0.00 | 0.00 | 0.00 | 0.00 | 0.00 | 0.00 | 0.00 |
| HSPA8 | 4.82 | 0.00 | 0.00 | 0.00 | 0.00 | 0.00 | 0.00 | 0.00 | 0.00 | 0.00 | 0.00 | 0.00 |
| HSPD1 | 5.67 | 0.00 | 0.00 | 0.00 | 0.00 | 0.00 | 0.00 | 0.00 | 0.00 | 0.00 | 0.00 | 0.00 |
| HSPE1 | 1.63 | 0.00 | 0.00 | 0.00 | 0.00 | 0.00 | 0.00 | 0.00 | 0.00 | 0.00 | 0.00 | 0.00 |
| HTR7 | 0.00 | 3.36 | 0.00 | 0.00 | 0.00 | 0.00 | 0.00 | 0.00 | 0.00 | 0.00 | 0.00 | 0.00 |
| ICAM1 | 0.00 | 0.00 | 0.00 | 0.00 | 0.00 | 0.00 | 0.00 | 0.00 | 0.00 | 0.00 | 0.00 | 0.00 |
| ICE1 | 0.00 | 0.00 | 0.00 | 0.00 | 0.00 | 0.00 | 0.00 | 0.00 | 0.00 | 0.00 | 0.00 | 0.00 |
| ICOS | 0.00 | 0.00 | 0.00 | 0.00 | 0.00 | 0.00 | 0.00 | 0.00 | 0.00 | 0.00 | 0.00 | 0.00 |
| ID1 | 0.00 | 0.00 | 0.00 | 0.00 | 0.00 | 0.00 | 0.00 | 0.00 | 0.00 | 0.00 | 0.00 | 2.96 |
| ID2 | 0.00 | 0.00 | 0.00 | 0.00 | 0.00 | 0.00 | 0.00 | 0.00 | 0.00 | 0.00 | 0.00 | 1.46 |
| ID3 | 0.00 | 0.00 | 0.00 | 0.00 | 0.00 | 0.00 | 0.00 | 0.00 | 0.00 | 0.00 | 0.00 | 0.00 |
| IER2 | 1.46 | 0.00 | 0.00 | 0.00 | 0.00 | 0.00 | 0.00 | 0.00 | 0.00 | 0.00 | 0.00 | 0.00 |
| IER3 | 0.00 | 0.00 | 0.00 | 0.00 | 0.00 | 0.00 | 0.00 | 0.00 | 0.00 | 0.00 | 0.00 | 16.10 |
| IFI203 | 0.00 | 0.00 | 0.00 | 0.00 | 0.00 | 0.00 | 0.00 | 0.00 | 0.00 | 0.00 | 0.00 | 0.00 |
| IFI204 | 0.00 | 0.00 | 0.00 | 0.00 | 0.00 | 0.00 | 7.35 | 0.00 | 0.00 | 0.00 | 0.00 | 0.00 |
| IFI205 | 0.00 | 2.33 | 4.31 | 0.00 | 0.00 | 0.00 | 4.48 | 0.00 | 1.52 | 0.00 | 0.00 | 0.00 |
| IFI27L2A | 0.00 | 1.39 | 0.00 | 0.00 | 0.00 | 3.38 | 1.72 | 0.00 | 1.62 | 5.38 | 0.00 | 0.00 |
| IFI35 | 0.00 | 0.00 | 0.00 | 0.00 | 0.00 | 0.00 | 1.31 | 0.00 | 0.00 | 0.00 | 0.00 | 0.00 |
| IFI47 | 0.00 | 0.00 | 0.00 | 0.00 | 0.00 | 0.00 | 2.78 | 0.00 | 0.00 | 0.00 | 0.00 | 0.00 |
| IFIT1 | 0.00 | 0.00 | 0.00 | 0.00 | 0.00 | 0.00 | 0.00 | 0.00 | 0.00 | 0.00 | 0.00 | 0.00 |
| IFIT1BL1 | 0.00 | 0.00 | 0.00 | 0.00 | 0.00 | 0.00 | 0.00 | 0.00 | 0.00 | 0.00 | 0.00 | 0.00 |
| IFIT3 | 0.00 | 2.45 | 0.00 | 0.00 | 0.00 | 0.00 | 0.00 | 0.00 | 1.39 | 0.00 | 0.00 | 0.00 |
| IFIT3B | 0.00 | 0.00 | 0.00 | 0.00 | 0.00 | 0.00 | 0.00 | 0.00 | 0.00 | 0.00 | 0.00 | 0.00 |
| IFITM1 | 0.00 | 0.00 | 1.47 | 0.00 | 0.00 | 0.00 | 0.00 | 0.00 | 0.00 | 0.00 | 0.00 | 6.31 |
| IFNGR1 | 0.00 | 0.00 | 0.00 | 0.00 | 0.00 | 0.00 | 0.00 | 0.00 | 0.00 | 0.00 | 0.00 | 0.00 |
| IFRD1 | 2.34 | 0.00 | 0.00 | 0.00 | 0.00 | 0.00 | 0.00 | 0.00 | 0.00 | 0.00 | 0.00 | 2.49 |
| IFT22 | 0.00 | 0.00 | 0.00 | 0.00 | 0.00 | 0.00 | 1.95 | 0.00 | 0.00 | 0.00 | 0.00 | 0.00 |
| IGTP | 0.00 | 0.00 | 0.00 | 0.00 | 0.00 | 0.00 | 0.00 | 0.00 | 0.00 | 0.00 | 0.00 | 0.00 |
| IIGP1 | 0.00 | 0.00 | 0.00 | 0.00 | 0.00 | 0.00 | 0.00 | 0.00 | 0.00 | 0.00 | 0.00 | 0.00 |
| IL18BP | 0.00 | 1.73 | 0.00 | 0.00 | 0.00 | 0.00 | 0.00 | 0.00 | 0.00 | 0.00 | 0.00 | 0.00 |
| IL1B | 0.00 | 0.00 | 0.00 | 0.00 | 0.00 | 0.00 | 0.00 | 0.00 | 0.00 | 0.00 | 0.00 | 1.65 |
| IL1R2 | 0.00 | 0.00 | 0.00 | 0.00 | 0.00 | 0.00 | 0.00 | 0.00 | 0.00 | 0.00 | 0.00 | 5.36 |
| IL1RL1 | 0.00 | 0.00 | 0.00 | 0.00 | 0.00 | 0.00 | 0.00 | 0.00 | 0.00 | 0.00 | 0.00 | 0.00 |
| IL1RN | 0.00 | 0.00 | 0.00 | 0.00 | 0.00 | 0.00 | 0.00 | 0.00 | 0.00 | 0.00 | 0.00 | 10.29 |
| IL4I1 | 0.00 | 0.00 | 0.00 | 0.00 | 0.00 | 0.00 | 3.02 | 0.00 | 0.00 | 0.00 | 0.00 | 0.00 |
| INTS5 | 0.00 | 1.79 | 0.00 | 0.00 | 0.00 | 0.00 | 0.00 | 0.00 | 0.00 | 0.00 | 0.00 | 0.00 |
| IQGAP2 | 0.00 | 0.00 | 0.00 | 0.00 | 0.00 | 0.00 | 0.00 | 0.00 | 0.00 | 0.00 | 0.00 | 0.00 |
| IRF7 | 0.00 | 2.29 | 0.00 | 0.00 | 0.00 | 0.00 | 3.71 | 0.00 | 4.27 | 0.00 | 0.00 | 0.00 |
| IRG1 | 0.00 | 0.00 | 0.00 | 0.00 | 0.00 | 0.00 | 0.00 | 0.00 | 0.00 | 0.00 | 0.00 | 13.09 |
| ISG15 | 0.00 | 4.20 | 3.61 | 1.31 | 0.00 | 0.00 | 7.35 | 1.74 | 2.34 | 0.00 | 0.00 | 0.00 |
| ISG20 | 0.00 | 0.00 | 0.00 | 0.00 | 0.00 | 0.00 | 1.53 | 0.00 | 0.00 | 0.00 | 0.00 | 0.00 |
| ITGA4 | 0.00 | 0.00 | 0.00 | 0.00 | 0.00 | 0.00 | 1.36 | 0.00 | 0.00 | 0.00 | 0.00 | 0.00 |
| ITGB7 | 0.00 | 0.00 | 0.00 | 0.00 | 0.00 | 0.00 | 0.00 | 0.00 | 0.00 | 0.00 | 0.00 | 0.00 |
| JADE1 | 0.00 | 0.00 | 0.00 | 0.00 | 0.00 | 0.00 | 0.00 | 0.00 | 0.00 | 0.00 | 0.00 | 0.00 |
| JUN | 5.39 | 0.00 | 0.00 | 0.00 | 0.00 | 0.00 | 0.00 | 0.00 | 0.00 | 0.00 | 0.00 | 0.00 |
| JUNB | 6.80 | 0.00 | 0.00 | 0.00 | 2.04 | 1.54 | 0.00 | 0.00 | 0.00 | 0.00 | 0.00 | 2.09 |
| KDM6B | 2.85 | 0.00 | 0.00 | 0.00 | 0.00 | 0.00 | 0.00 | 0.00 | 0.00 | 0.00 | 0.00 | 0.00 |
| KDR | 0.00 | 0.00 | 0.00 | 0.00 | 0.00 | 0.00 | 1.31 | 0.00 | 0.00 | 0.00 | 0.00 | 0.00 |
| KIFC1 | 0.00 | 0.00 | 0.00 | 0.00 | 0.00 | 0.00 | 0.00 | 0.00 | 0.00 | 0.00 | 0.00 | 0.00 |
| KIT | 0.00 | 0.00 | 0.00 | 0.00 | 0.00 | 0.00 | 0.00 | 0.00 | 0.00 | 0.00 | 0.00 | 1.48 |
| KLF2 | 1.63 | 0.00 | 0.00 | 0.00 | 0.00 | 0.00 | 0.00 | 0.00 | 0.00 | 0.00 | 0.00 | 0.00 |
| KLF3 | 0.00 | 0.00 | 0.00 | 0.00 | 0.00 | 0.00 | 0.00 | 0.00 | 0.00 | 0.00 | 0.00 | 0.00 |
| KLF9 | 0.00 | 1.42 | 0.00 | 0.00 | 0.00 | 0.00 | 0.00 | 0.00 | 0.00 | 0.00 | 0.00 | 0.00 |
| KLRA1 | 0.00 | 0.00 | 0.00 | 0.00 | 0.00 | 0.00 | 0.00 | 0.00 | 0.00 | 0.00 | 0.00 | 0.00 |
| KLRA8 | 0.00 | 0.00 | 0.00 | 0.00 | 0.00 | 0.00 | 0.00 | 0.00 | 0.00 | 0.00 | 0.00 | 0.00 |
| KLRA9 | 0.00 | 0.00 | 0.00 | 0.00 | 0.00 | 0.00 | 0.00 | 0.00 | 0.00 | 0.00 | 0.00 | 0.00 |
| KLRB1B | 0.00 | 0.00 | 0.00 | 0.00 | 0.00 | 0.00 | 1.35 | 0.00 | 0.00 | 0.00 | 0.00 | 0.00 |
| KLRC1 | 0.00 | 0.00 | 0.00 | 0.00 | 0.00 | 0.00 | 0.00 | 0.00 | 0.00 | 0.00 | 0.00 | 0.00 |
| KLRD1 | 0.00 | 0.00 | 0.00 | 0.00 | 0.00 | 0.00 | 0.00 | 0.00 | 0.00 | 0.00 | 0.00 | 0.00 |
| KLRG1 | 0.00 | 0.00 | 1.55 | 0.00 | 0.00 | 0.00 | 0.00 | 0.00 | 0.00 | 0.00 | 0.00 | 0.00 |
| KLRK1 | 0.00 | 0.00 | 0.00 | 0.00 | 0.00 | 0.00 | 1.67 | 0.00 | 0.00 | 0.00 | 0.00 | 0.00 |
| KMO | 0.00 | 2.47 | 0.00 | 0.00 | 0.00 | 0.00 | 0.00 | 0.00 | 0.00 | 0.00 | 0.00 | 0.00 |
| KPNA2 | 0.00 | 0.00 | 0.00 | 0.00 | 0.00 | 0.00 | 0.00 | 0.00 | 0.00 | 0.00 | 0.00 | 0.00 |
| LDHA | 1.42 | 0.00 | 0.00 | 0.00 | 0.00 | 0.00 | 0.00 | 0.00 | 0.00 | 0.00 | 0.00 | 2.07 |
| LEPR | 0.00 | 0.00 | 0.00 | 1.89 | 0.00 | 0.00 | 0.00 | 0.00 | 1.47 | 0.00 | 0.00 | 1.90 |
| LFNG | 0.00 | 0.00 | 0.00 | 0.00 | 0.00 | 0.00 | 0.00 | 0.00 | 0.00 | 0.00 | 0.00 | 0.00 |
| LGALS3BP | 0.00 | 1.36 | 0.00 | 0.00 | 0.00 | 0.00 | 0.00 | 0.00 | 0.00 | 0.00 | 0.00 | 0.00 |
| LILR4B | 0.00 | 0.00 | 0.00 | 0.00 | 0.00 | 0.00 | 0.00 | 0.00 | 0.00 | 0.00 | 0.00 | 4.65 |
| LIPA | 0.00 | 0.00 | 0.00 | 0.00 | 0.00 | 0.00 | 0.00 | 0.00 | 0.00 | 0.00 | 0.00 | 0.00 |
| LITAF | 0.00 | 0.00 | 0.00 | 0.00 | 0.00 | 0.00 | 0.00 | 0.00 | 0.00 | 0.00 | 0.00 | 2.88 |
| LMBRD2 | 0.00 | 0.00 | 0.00 | 0.00 | 0.00 | 0.00 | 0.00 | 0.00 | 0.00 | 0.00 | 0.00 | 0.00 |
| LMNB1 | 0.00 | 0.00 | 0.00 | 0.00 | 0.00 | 0.00 | 0.00 | 0.00 | 0.00 | 0.00 | 0.00 | 4.65 |
| LNPEP | 0.00 | 0.00 | 0.00 | 0.00 | 0.00 | 0.00 | 0.00 | 0.00 | 0.00 | 0.00 | 0.00 | 0.00 |
| LRG1 | 0.00 | 0.00 | 0.00 | 0.00 | 0.00 | 0.00 | 0.00 | 0.00 | 0.00 | 0.00 | 0.00 | 5.47 |
| LRRC25 | 0.00 | 0.00 | 0.00 | 0.00 | 0.00 | 0.00 | 0.00 | 0.00 | 1.70 | 0.00 | 0.00 | 0.00 |
| LTA | 0.00 | 0.00 | 0.00 | 0.00 | 0.00 | 1.48 | 0.00 | 0.00 | 0.00 | 0.00 | 0.00 | 0.00 |
| LTB4R1 | 0.00 | 0.00 | 0.00 | 0.00 | 0.00 | 0.00 | 0.00 | 0.00 | 0.00 | 0.00 | 0.00 | 0.00 |
| LY6A | 0.00 | 2.81 | 0.00 | 0.00 | 0.00 | 0.00 | 2.68 | 0.00 | 3.84 | 0.00 | 0.00 | 0.00 |
| LY6C2 | 0.00 | 2.47 | 0.00 | 0.00 | 0.00 | 0.00 | 0.00 | 0.00 | 4.06 | 0.00 | 0.00 | 2.41 |
| LY6D | 0.00 | 0.00 | 4.37 | 0.00 | 0.00 | 0.00 | 0.00 | 0.00 | 0.00 | 0.00 | 0.00 | 0.00 |
| LY6E | 0.00 | 1.86 | 0.00 | 0.00 | 0.00 | 0.00 | 0.00 | 0.00 | 1.48 | 0.00 | 0.00 | 0.00 |
| LYZ2 | 0.00 | 0.00 | 0.00 | 10.19 | 0.00 | 0.00 | 0.00 | 0.00 | 0.00 | 0.00 | 0.00 | 0.00 |
| MACROD1 | 0.00 | 0.00 | 0.00 | 0.00 | 0.00 | 0.00 | 0.00 | 0.00 | 0.00 | 0.00 | 0.00 | 0.00 |
| MAFF | 0.00 | 0.00 | 0.00 | 0.00 | 0.00 | 0.00 | 0.00 | 0.00 | 0.00 | 0.00 | 0.00 | 1.58 |
| MAP3K8 | 2.80 | 0.00 | 0.00 | 0.00 | 0.00 | 0.00 | 0.00 | 0.00 | 0.00 | 0.00 | 0.00 | 0.00 |
| MARCKS | 0.00 | 0.00 | 0.00 | 2.35 | 0.00 | 0.00 | 0.00 | 0.00 | 0.00 | 0.00 | 0.00 | 0.00 |
| MARCKSL1 | 2.48 | 0.00 | 0.00 | 0.00 | 0.00 | 0.00 | 1.36 | 0.00 | 0.00 | 0.00 | 0.00 | 0.00 |
| MASTL | 0.00 | 0.00 | 0.00 | 0.00 | 0.00 | 3.19 | 0.00 | 0.00 | 0.00 | 0.00 | 0.00 | 0.00 |
| MCL1 | 0.00 | 0.00 | 0.00 | 0.00 | 0.00 | 0.00 | 0.00 | 0.00 | 0.00 | 0.00 | 0.00 | 1.32 |
| MCTP2 | 0.00 | 0.00 | 0.00 | 0.00 | 0.00 | 0.00 | 0.00 | 0.00 | 0.00 | 0.00 | 0.00 | 1.54 |
| MED13 | 0.00 | 0.00 | 0.00 | 0.00 | 0.00 | 0.00 | 0.00 | 0.00 | 0.00 | 0.00 | 0.00 | 0.00 |
| MEF2C | 0.00 | 0.00 | 0.00 | 0.00 | 0.00 | 0.00 | 0.00 | 0.00 | 0.00 | 0.00 | 0.00 | 0.00 |
| METTL14 | 0.00 | 0.00 | 0.00 | 0.00 | 0.00 | 0.00 | 0.00 | 0.00 | 0.00 | 0.00 | 0.00 | 0.00 |
| METTL9 | 0.00 | 0.00 | 0.00 | 0.00 | 0.00 | 0.00 | 1.30 | 0.00 | 0.00 | 0.00 | 0.00 | 0.00 |
| MIF | 1.53 | 0.00 | 0.00 | 0.00 | 0.00 | 0.00 | 0.00 | 0.00 | 0.00 | 0.00 | 0.00 | 3.82 |
| MIRT1 | 0.00 | 0.00 | 0.00 | 0.00 | 0.00 | 0.00 | 0.00 | 0.00 | 0.00 | 0.00 | 0.00 | 0.00 |
| MLANA | 0.00 | 1.37 | 0.00 | 0.00 | 1.53 | 2.07 | 0.00 | 0.00 | 0.00 | 0.00 | 0.00 | 0.00 |
| MLKL | 0.00 | 1.35 | 0.00 | 0.00 | 0.00 | 0.00 | 0.00 | 0.00 | 1.47 | 0.00 | 0.00 | 0.00 |
| MMADHC | 0.00 | 0.00 | 0.00 | 0.00 | 0.00 | 0.00 | 0.00 | 0.00 | 0.00 | 0.00 | 0.00 | 0.00 |
| MMP12 | 0.00 | 0.00 | 0.00 | 0.00 | 0.00 | 0.00 | 0.00 | 1.30 | 0.00 | 0.00 | 0.00 | 0.00 |
| MMP19 | 0.00 | 0.00 | 0.00 | 0.00 | 0.00 | 1.83 | 0.00 | 0.00 | 0.00 | 0.00 | 0.00 | 0.00 |
| MMP8 | 0.00 | 0.00 | 0.00 | 0.00 | 0.00 | 0.00 | 0.00 | 0.00 | 0.00 | 0.00 | 0.00 | 1.86 |
| MMP9 | 0.00 | 0.00 | 0.00 | 0.00 | 0.00 | 0.00 | 0.00 | 0.00 | 0.00 | 0.00 | 0.00 | 2.63 |
| MNDA | 0.00 | 0.00 | 0.00 | 0.00 | 0.00 | 0.00 | 3.74 | 0.00 | 1.59 | 0.00 | 0.00 | 0.00 |
| MNDAL | 0.00 | 1.39 | 0.00 | 0.00 | 0.00 | 0.00 | 0.00 | 0.00 | 2.12 | 0.00 | 0.00 | 0.00 |
| MPEG1 | 0.00 | 0.00 | 0.00 | 0.00 | 0.00 | 0.00 | 0.00 | 0.00 | 0.00 | 0.00 | 0.00 | 0.00 |
| MPP6 | 0.00 | 0.00 | 0.00 | 0.00 | 0.00 | 0.00 | 0.00 | 0.00 | 0.00 | 0.00 | 0.00 | 0.00 |
| MS4A4B | 0.00 | 0.00 | 0.00 | 0.00 | 0.00 | 0.00 | 0.00 | 0.00 | 0.00 | 0.00 | 0.00 | 0.00 |
| MS4A4C | 0.00 | 1.73 | 0.00 | 0.00 | 0.00 | 0.00 | 6.03 | 0.00 | 1.70 | 0.00 | 0.00 | 0.00 |
| MS4A6B | 0.00 | 0.00 | 0.00 | 0.00 | 0.00 | 0.00 | 1.47 | 0.00 | 0.00 | 0.00 | 0.00 | 0.00 |
| MS4A6C | 0.00 | 0.00 | 0.00 | 0.00 | 0.00 | 3.93 | 0.00 | 0.00 | 0.00 | 0.00 | 0.00 | 0.00 |
| MT1 | 0.00 | 0.00 | 0.00 | 0.00 | 0.00 | 0.00 | 0.00 | 0.00 | 0.00 | 0.00 | 0.00 | 0.00 |
| MT-ATP8 | 0.00 | 1.63 | 0.00 | 0.00 | 0.00 | 0.00 | 1.83 | 0.00 | 0.00 | 0.00 | 0.00 | 0.00 |
| MTFP1 | 1.63 | 0.00 | 0.00 | 0.00 | 0.00 | 0.00 | 0.00 | 0.00 | 0.00 | 0.00 | 0.00 | 0.00 |
| MVP | 0.00 | 0.00 | 0.00 | 0.00 | 0.00 | 0.00 | 1.37 | 0.00 | 0.00 | 0.00 | 0.00 | 0.00 |
| MX1 | 0.00 | 1.63 | 0.00 | 0.00 | 0.00 | 0.00 | 0.00 | 0.00 | 0.00 | 0.00 | 0.00 | 0.00 |
| MXD1 | 0.00 | 0.00 | 0.00 | 0.00 | 0.00 | 0.00 | 0.00 | 0.00 | 0.00 | 0.00 | 0.00 | 6.27 |
| MYC | 3.20 | 0.00 | 0.00 | 0.00 | 0.00 | 0.00 | 0.00 | 0.00 | 0.00 | 0.00 | 0.00 | 0.00 |
| MYO1G | 0.00 | 0.00 | 0.00 | 0.00 | 0.00 | 0.00 | 1.83 | 0.00 | 0.00 | 0.00 | 0.00 | 0.00 |
| MZB1 | 0.00 | 0.00 | 0.00 | 0.00 | 0.00 | 0.00 | 0.00 | 0.00 | 0.00 | 0.00 | 0.00 | 0.00 |
| NABP1 | 0.00 | 0.00 | 0.00 | 0.00 | 0.00 | 0.00 | 0.00 | 0.00 | 0.00 | 0.00 | 0.00 | 0.00 |
| NAPSA | 0.00 | 0.00 | 0.00 | 0.00 | 0.00 | 0.00 | 0.00 | 0.00 | 0.00 | 0.00 | 0.00 | 0.00 |
| NCF2 | 0.00 | 0.00 | 0.00 | 0.00 | 0.00 | 0.00 | 0.00 | 0.00 | 0.00 | 0.00 | 0.00 | 0.00 |
| NCOA7 | 0.00 | 1.39 | 0.00 | 0.00 | 0.00 | 0.00 | 0.00 | 0.00 | 0.00 | 0.00 | 0.00 | 0.00 |
| NEAT1 | 3.17 | 0.00 | 0.00 | 0.00 | 0.00 | 2.22 | 0.00 | 0.00 | 0.00 | 0.00 | 0.00 | 0.00 |
| NFKBIA | 2.16 | 0.00 | 0.00 | 0.00 | 0.00 | 2.22 | 0.00 | 0.00 | 0.00 | 0.00 | 0.00 | 2.79 |
| NFKBID | 1.41 | 0.00 | 0.00 | 0.00 | 0.00 | 0.00 | 0.00 | 0.00 | 0.00 | 0.00 | 0.00 | 0.00 |
| NINJ1 | 0.00 | 0.00 | 0.00 | 0.00 | 0.00 | 0.00 | 0.00 | 0.00 | 0.00 | 0.00 | 0.00 | 1.97 |
| NKG7 | 0.00 | 0.00 | 0.00 | 0.00 | 0.00 | 0.00 | 0.00 | 0.00 | 0.00 | 0.00 | 0.00 | 0.00 |
| NLRC5 | 0.00 | 1.39 | 0.00 | 0.00 | 0.00 | 0.00 | 0.00 | 0.00 | 0.00 | 0.00 | 0.00 | 0.00 |
| NMT1 | 0.00 | 0.00 | 0.00 | 0.00 | 0.00 | 0.00 | 0.00 | 0.00 | 0.00 | 0.00 | 0.00 | 0.00 |
| NOCT | 0.00 | 0.00 | 0.00 | 0.00 | 0.00 | 0.00 | 0.00 | 0.00 | 0.00 | 0.00 | 0.00 | 2.12 |
| NPEPPS | 0.00 | 0.00 | 0.00 | 0.00 | 0.00 | 0.00 | 0.00 | 0.00 | 0.00 | 0.00 | 0.00 | 1.88 |
| NT5C3 | 0.00 | 0.00 | 0.00 | 0.00 | 0.00 | 0.00 | 0.00 | 0.00 | 0.00 | 0.00 | 0.00 | 0.00 |
| NUDT4 | 0.00 | 0.00 | 0.00 | 0.00 | 0.00 | 0.00 | 0.00 | 0.00 | 0.00 | 0.00 | 0.00 | 2.05 |
| OAS1A | 0.00 | 0.00 | 0.00 | 0.00 | 0.00 | 0.00 | 2.04 | 0.00 | 0.00 | 0.00 | 0.00 | 0.00 |
| OAS1G | 0.00 | 0.00 | 0.00 | 0.00 | 0.00 | 0.00 | 2.04 | 0.00 | 0.00 | 0.00 | 0.00 | 0.00 |
| OAS3 | 0.00 | 0.00 | 0.00 | 0.00 | 0.00 | 0.00 | 0.00 | 0.00 | 1.59 | 0.00 | 0.00 | 0.00 |
| OASL1 | 0.00 | 0.00 | 0.00 | 0.00 | 0.00 | 0.00 | 2.96 | 0.00 | 0.00 | 0.00 | 0.00 | 0.00 |
| OASL2 | 0.00 | 3.24 | 0.00 | 0.00 | 0.00 | 0.00 | 3.02 | 0.00 | 1.96 | 0.00 | 0.00 | 0.00 |
| OCSTAMP | 0.00 | 0.00 | 0.00 | 0.00 | 0.00 | 0.00 | 0.00 | 0.00 | 0.00 | 0.00 | 0.00 | 2.38 |
| ODC1 | 0.00 | 0.00 | 0.00 | 0.00 | 0.00 | 0.00 | 0.00 | 0.00 | 0.00 | 0.00 | 0.00 | 0.00 |
| OSM | 0.00 | 0.00 | 0.00 | 0.00 | 0.00 | 0.00 | 0.00 | 0.00 | 0.00 | 0.00 | 0.00 | 18.21 |
| P2RY14 | 0.00 | 1.40 | 0.00 | 0.00 | 0.00 | 0.00 | 0.00 | 0.00 | 0.00 | 0.00 | 0.00 | 0.00 |
| P2RY6 | 0.00 | 0.00 | 0.00 | 0.00 | 0.00 | 0.00 | 0.00 | 0.00 | 0.00 | 0.00 | 0.00 | 0.00 |
| PARP12 | 0.00 | 2.47 | 0.00 | 0.00 | 0.00 | 0.00 | 1.51 | 0.00 | 0.00 | 0.00 | 0.00 | 0.00 |
| PARP14 | 0.00 | 2.01 | 0.00 | 0.00 | 0.00 | 0.00 | 1.76 | 0.00 | 0.00 | 0.00 | 0.00 | 0.00 |
| PCMTD1 | 0.00 | 0.00 | 0.00 | 0.00 | 0.00 | 0.00 | 0.00 | 0.00 | 0.00 | 0.00 | 0.00 | 0.00 |
| PCYOX1L | 0.00 | 0.00 | 0.00 | 0.00 | 0.00 | 0.00 | 0.00 | 0.00 | 0.00 | 0.00 | 0.00 | 0.00 |
| PGGT1B | 0.00 | 0.00 | 0.00 | 0.00 | 0.00 | 0.00 | 0.00 | 0.00 | 0.00 | 0.00 | 0.00 | 0.00 |
| PGLYRP1 | 0.00 | 2.63 | 0.00 | 0.00 | 0.00 | 0.00 | 0.00 | 0.00 | 0.00 | 0.00 | 0.00 | 0.00 |
| PHF11B | 0.00 | 2.54 | 2.13 | 0.00 | 0.00 | 0.00 | 2.58 | 0.00 | 0.00 | 0.00 | 0.00 | 0.00 |
| PHF11C | 0.00 | 0.00 | 0.00 | 0.00 | 0.00 | 0.00 | 0.00 | 0.00 | 0.00 | 0.00 | 0.00 | 0.00 |
| PHF11D | 0.00 | 3.82 | 0.00 | 0.00 | 0.00 | 0.00 | 3.74 | 0.00 | 1.59 | 0.00 | 0.00 | 0.00 |
| PHLDA1 | 9.40 | 0.00 | 1.51 | 0.00 | 0.00 | 0.00 | 0.00 | 0.00 | 0.00 | 0.00 | 0.00 | 0.00 |
| PIGL | 0.00 | 0.00 | 0.00 | 0.00 | 0.00 | 0.00 | 1.52 | 0.00 | 0.00 | 0.00 | 0.00 | 0.00 |
| PIK3R6 | 0.00 | 0.00 | 0.00 | 0.00 | 0.00 | 0.00 | 0.00 | 0.00 | 0.00 | 0.00 | 0.00 | 0.00 |
| PILRA | 0.00 | 0.00 | 0.00 | 0.00 | 0.00 | 0.00 | 1.31 | 0.00 | 0.00 | 0.00 | 0.00 | 0.00 |
| PIM1 | 0.00 | 0.00 | 0.00 | 0.00 | 0.00 | 0.00 | 0.00 | 0.00 | 0.00 | 0.00 | 0.00 | 2.23 |
| PJA1 | 0.00 | 0.00 | 0.00 | 0.00 | 0.00 | 0.00 | 0.00 | 0.00 | 0.00 | 0.00 | 0.00 | 0.00 |
| PKIB | 0.00 | 0.00 | 0.00 | 0.00 | 0.00 | 0.00 | 0.00 | 0.00 | 0.00 | 0.00 | 0.00 | 0.00 |
| PLAC8 | 0.00 | 0.00 | 0.00 | 0.00 | 0.00 | 0.00 | 0.00 | 0.00 | 0.00 | 0.00 | 0.00 | 0.00 |
| PLAUR | 0.00 | 0.00 | 0.00 | 0.00 | 0.00 | 0.00 | 0.00 | 0.00 | 1.52 | 0.00 | 0.00 | 2.19 |
| PLBD1 | 0.00 | 0.00 | 0.00 | 0.00 | 0.00 | 0.00 | 0.00 | 0.00 | 0.00 | 0.00 | 0.00 | 0.00 |
| PLIN2 | 0.00 | 0.00 | 0.00 | 0.00 | 0.00 | 0.00 | 0.00 | 0.00 | 0.00 | 0.00 | 0.00 | 1.89 |
| PLK1 | 0.00 | 1.53 | 0.00 | 0.00 | 0.00 | 0.00 | 0.00 | 0.00 | 0.00 | 0.00 | 0.00 | 0.00 |
| PLK2 | 2.41 | 0.00 | 0.00 | 0.00 | 0.00 | 0.00 | 0.00 | 0.00 | 0.00 | 0.00 | 0.00 | 0.00 |
| PLK3 | 0.00 | 0.00 | 0.00 | 0.00 | 0.00 | 0.00 | 0.00 | 0.00 | 0.00 | 0.00 | 0.00 | 2.55 |
| PLSCR1 | 0.00 | 0.00 | 0.00 | 0.00 | 0.00 | 0.00 | 0.00 | 0.00 | 0.00 | 0.00 | 0.00 | 2.87 |
| PLTP | 0.00 | 1.74 | 0.00 | 0.00 | 0.00 | 0.00 | 0.00 | 0.00 | 0.00 | 0.00 | 0.00 | 0.00 |
| PMAIP1 | 2.18 | 0.00 | 0.00 | 0.00 | 0.00 | 0.00 | 0.00 | 0.00 | 0.00 | 0.00 | 0.00 | 0.00 |
| PMEL | 0.00 | 0.00 | 0.00 | 0.00 | 0.00 | 0.00 | 0.00 | 0.00 | 0.00 | 0.00 | 0.00 | 0.00 |
| PML | 0.00 | 0.00 | 0.00 | 0.00 | 0.00 | 0.00 | 0.00 | 0.00 | 2.34 | 0.00 | 0.00 | 0.00 |
| PNP | 0.00 | 1.85 | 0.00 | 0.00 | 0.00 | 0.00 | 0.00 | 0.00 | 0.00 | 0.00 | 0.00 | 0.00 |
| PNRC1 | 0.00 | 0.00 | 0.00 | 0.00 | 0.00 | 0.00 | 0.00 | 0.00 | 0.00 | 0.00 | 0.00 | 1.62 |
| POLE4 | 0.00 | 1.39 | 0.00 | 0.00 | 0.00 | 0.00 | 0.00 | 0.00 | 0.00 | 0.00 | 0.00 | 0.00 |
| PPP1R10 | 0.00 | 1.33 | 0.00 | 0.00 | 0.00 | 0.00 | 0.00 | 0.00 | 0.00 | 0.00 | 0.00 | 0.00 |
| PPP1R15A | 8.69 | 0.00 | 1.46 | 0.00 | 1.66 | 0.00 | 0.00 | 0.00 | 1.68 | 0.00 | 0.00 | 2.07 |
| PPP1R16B | 5.15 | 0.00 | 0.00 | 0.00 | 0.00 | 0.00 | 0.00 | 0.00 | 0.00 | 0.00 | 0.00 | 0.00 |
| PPP2R3C | 0.00 | 0.00 | 0.00 | 0.00 | 0.00 | 0.00 | 0.00 | 0.00 | 0.00 | 0.00 | 0.00 | 2.05 |
| PPP6R3 | 1.56 | 0.00 | 0.00 | 0.00 | 0.00 | 0.00 | 0.00 | 0.00 | 0.00 | 0.00 | 0.00 | 0.00 |
| PRDM2 | 0.00 | 0.00 | 0.00 | 0.00 | 0.00 | 0.00 | 1.57 | 0.00 | 0.00 | 0.00 | 0.00 | 0.00 |
| PRDX6 | 0.00 | 0.00 | 0.00 | 0.00 | 0.00 | 0.00 | 0.00 | 0.00 | 0.00 | 0.00 | 0.00 | 1.50 |
| PRKCA | 0.00 | 0.00 | 0.00 | 0.00 | 0.00 | 0.00 | 1.50 | 0.00 | 0.00 | 0.00 | 0.00 | 0.00 |
| PSME2 | 0.00 | 0.00 | 0.00 | 0.00 | 0.00 | 0.00 | 0.00 | 0.00 | 0.00 | 0.00 | 0.00 | 0.00 |
| PTAFR | 0.00 | 0.00 | 0.00 | 0.00 | 0.00 | 1.75 | 0.00 | 0.00 | 0.00 | 0.00 | 0.00 | 3.35 |
| PTGDS | 0.00 | 0.00 | 0.00 | 0.00 | 0.00 | 0.00 | 1.50 | 0.00 | 0.00 | 0.00 | 0.00 | 0.00 |
| PTGS2 | 0.00 | 0.00 | 0.00 | 0.00 | 0.00 | 0.00 | 0.00 | 0.00 | 0.00 | 0.00 | 0.00 | 7.60 |
| PTP4A1 | 3.10 | 0.00 | 0.00 | 0.00 | 0.00 | 0.00 | 0.00 | 0.00 | 0.00 | 0.00 | 0.00 | 2.53 |
| PTPN22 | 2.28 | 0.00 | 0.00 | 0.00 | 0.00 | 0.00 | 1.85 | 0.00 | 0.00 | 0.00 | 0.00 | 0.00 |
| PVT1 | 0.00 | 0.00 | 0.00 | 0.00 | 0.00 | 0.00 | 0.00 | 0.00 | 0.00 | 0.00 | 0.00 | 0.00 |
| PYDC3 | 0.00 | 2.83 | 0.00 | 0.00 | 0.00 | 0.00 | 0.00 | 0.00 | 2.66 | 0.00 | 0.00 | 0.00 |
| PYDC4 | 0.00 | 2.13 | 0.00 | 0.00 | 0.00 | 0.00 | 1.78 | 1.53 | 2.29 | 0.00 | 0.00 | 0.00 |
| PYHIN1 | 0.00 | 1.83 | 0.00 | 0.00 | 0.00 | 0.00 | 2.04 | 0.00 | 0.00 | 0.00 | 0.00 | 0.00 |
| RAB10OS | 0.00 | 0.00 | 0.00 | 0.00 | 0.00 | 0.00 | 0.00 | 0.00 | 0.00 | 0.00 | 0.00 | 0.00 |
| RAB28 | 0.00 | 0.00 | 0.00 | 0.00 | 0.00 | 0.00 | 0.00 | 0.00 | 0.00 | 0.00 | 0.00 | 0.00 |
| RAB3IL1 | 0.00 | 0.00 | 0.00 | 0.00 | 0.00 | 0.00 | 0.00 | 0.00 | 0.00 | 0.00 | 0.00 | 0.00 |
| RAMP1 | 0.00 | 0.00 | 0.00 | 0.00 | 0.00 | 0.00 | 0.00 | 0.00 | 0.00 | 0.00 | 0.00 | 0.00 |
| RBM39 | 0.00 | 0.00 | 0.00 | 0.00 | 0.00 | 1.75 | 0.00 | 0.00 | 0.00 | 0.00 | 0.00 | 1.54 |
| RBPJ | 0.00 | 0.00 | 1.38 | 0.00 | 0.00 | 0.00 | 0.00 | 0.00 | 0.00 | 0.00 | 0.00 | 0.00 |
| RETNLG | 9.87 | 0.00 | 0.00 | 0.00 | 0.00 | 5.28 | 0.00 | 0.00 | 0.00 | 0.00 | 0.00 | 14.16 |
| RGCC | 0.00 | 0.00 | 0.00 | 0.00 | 0.00 | 0.00 | 0.00 | 0.00 | 0.00 | 0.00 | 0.00 | 4.56 |
| RGL1 | 0.00 | 2.33 | 0.00 | 0.00 | 0.00 | 0.00 | 0.00 | 0.00 | 0.00 | 0.00 | 0.00 | 0.00 |
| RGS1 | 0.00 | 0.00 | 0.00 | 0.00 | 0.00 | 0.00 | 0.00 | 0.00 | 0.00 | 0.00 | 0.00 | 1.88 |
| RGS16 | 0.00 | 0.00 | 0.00 | 0.00 | 0.00 | 0.00 | 0.00 | 0.00 | 0.00 | 0.00 | 0.00 | 0.00 |
| RHOV | 0.00 | 0.00 | 0.00 | 0.00 | 0.00 | 0.00 | 0.00 | 0.00 | 2.52 | 0.00 | 0.00 | 6.68 |
| RIF1 | 0.00 | 1.63 | 0.00 | 0.00 | 0.00 | 0.00 | 0.00 | 0.00 | 0.00 | 0.00 | 0.00 | 0.00 |
| RIN2 | 0.00 | 2.11 | 0.00 | 0.00 | 0.00 | 0.00 | 0.00 | 0.00 | 0.00 | 0.00 | 0.00 | 0.00 |
| RINL | 0.00 | 0.00 | 0.00 | 0.00 | 0.00 | 0.00 | 0.00 | 0.00 | 0.00 | 0.00 | 0.00 | 0.00 |
| RNASE6 | 0.00 | 0.00 | 0.00 | 0.00 | 0.00 | 0.00 | 0.00 | 0.00 | 0.00 | 0.00 | 0.00 | 0.00 |
| RNF138 | 0.00 | 0.00 | 0.00 | 0.00 | 0.00 | 0.00 | 0.00 | 0.00 | 0.00 | 0.00 | 0.00 | 0.00 |
| RNF213 | 0.00 | 0.00 | 0.00 | 0.00 | 0.00 | 0.00 | 0.00 | 0.00 | 2.66 | 0.00 | 0.00 | 0.00 |
| RNF34 | 0.00 | 2.01 | 0.00 | 0.00 | 0.00 | 0.00 | 0.00 | 0.00 | 0.00 | 0.00 | 0.00 | 0.00 |
| ROCK2 | 0.00 | 0.00 | 0.00 | 0.00 | 0.00 | 0.00 | 0.00 | 0.00 | 0.00 | 0.00 | 0.00 | 0.00 |
| RP23-6I17.1 | 0.00 | 0.00 | 0.00 | 0.00 | 0.00 | 0.00 | 0.00 | 2.06 | 0.00 | 0.00 | 0.00 | 2.15 |
| RPIA | 2.40 | 0.00 | 0.00 | 0.00 | 0.00 | 0.00 | 0.00 | 0.00 | 0.00 | 0.00 | 0.00 | 0.00 |
| RPL35 | 1.63 | 0.00 | 0.00 | 0.00 | 0.00 | 1.88 | 0.00 | 0.00 | 0.00 | 0.00 | 0.00 | 0.00 |
| RPL36-PS3 | 1.63 | 0.00 | 0.00 | 0.00 | 0.00 | 0.00 | 0.00 | 0.00 | 0.00 | 0.00 | 0.00 | 0.00 |
| RTP4 | 0.00 | 3.24 | 0.00 | 0.00 | 0.00 | 0.00 | 4.66 | 0.00 | 3.80 | 0.00 | 0.00 | 0.00 |
| RUFY3 | 1.35 | 0.00 | 0.00 | 0.00 | 0.00 | 0.00 | 0.00 | 0.00 | 0.00 | 0.00 | 0.00 | 0.00 |
| S100A11 | 0.00 | 0.00 | 0.00 | 0.00 | 0.00 | 0.00 | 0.00 | 0.00 | 0.00 | 0.00 | 0.00 | 2.69 |
| S100A6 | 0.00 | 0.00 | 0.00 | 0.00 | 0.00 | 0.00 | 0.00 | 0.00 | 0.00 | 0.00 | 0.00 | 0.00 |
| S100A9 | 15.38 | 0.00 | 0.00 | 0.00 | 0.00 | 5.28 | 0.00 | 5.12 | 0.00 | 0.00 | 1.34 | 30.96 |
| SACS | 0.00 | 0.00 | 0.00 | 0.00 | 0.00 | 0.00 | 0.00 | 0.00 | 0.00 | 0.00 | 0.00 | 0.00 |
| SAG | 0.00 | 0.00 | 0.00 | 0.00 | 0.00 | 0.00 | 1.32 | 0.00 | 0.00 | 0.00 | 0.00 | 0.00 |
| SAMHD1 | 0.00 | 0.00 | 0.00 | 0.00 | 0.00 | 0.00 | 1.54 | 0.00 | 0.00 | 0.00 | 0.00 | 0.00 |
| SAP30L | 0.00 | 0.00 | 0.00 | 0.00 | 0.00 | 0.00 | 0.00 | 0.00 | 0.00 | 0.00 | 0.00 | 0.00 |
| SARAF | 0.00 | 0.00 | 0.00 | 0.00 | 0.00 | 0.00 | 1.42 | 0.00 | 0.00 | 0.00 | 0.00 | 0.00 |
| SATB1 | 1.63 | 0.00 | 0.00 | 0.00 | 0.00 | 0.00 | 0.00 | 0.00 | 0.00 | 0.00 | 0.00 | 0.00 |
| SDC3 | 0.00 | 2.81 | 0.00 | 0.00 | 0.00 | 0.00 | 2.13 | 0.00 | 3.28 | 0.00 | 0.00 | 0.00 |
| SDC4 | 0.00 | 0.00 | 0.00 | 0.00 | 0.00 | 0.00 | 0.00 | 0.00 | 0.00 | 0.00 | 0.00 | 0.00 |
| SDHAF1 | 3.19 | 0.00 | 0.00 | 0.00 | 0.00 | 0.00 | 0.00 | 0.00 | 0.00 | 0.00 | 0.00 | 0.00 |
| SEC22A | 0.00 | 0.00 | 0.00 | 0.00 | 0.00 | 0.00 | 0.00 | 0.00 | 0.00 | 0.00 | 0.00 | 1.48 |
| SELENBP1 | 0.00 | 0.00 | 0.00 | 0.00 | 0.00 | 0.00 | 0.00 | 0.00 | 0.00 | 0.00 | 0.00 | 0.00 |
| SENP6 | 0.00 | 0.00 | 0.00 | 0.00 | 0.00 | 0.00 | 0.00 | 0.00 | 0.00 | 0.00 | 0.00 | 0.00 |
| SEPT11 | 0.00 | 0.00 | 0.00 | 0.00 | 0.00 | 0.00 | 0.00 | 0.00 | 0.00 | 0.00 | 0.00 | 0.00 |
| SERINC3 | 1.93 | 0.00 | 0.00 | 0.00 | 0.00 | 0.00 | 0.00 | 0.00 | 0.00 | 0.00 | 0.00 | 0.00 |
| SERTAD1 | 0.00 | 0.00 | 0.00 | 0.00 | 0.00 | 0.00 | 0.00 | 0.00 | 0.00 | 0.00 | 0.00 | 1.64 |
| SETX | 0.00 | 0.00 | 0.00 | 0.00 | 0.00 | 0.00 | 0.00 | 0.00 | 0.00 | 0.00 | 0.00 | 0.00 |
| SGK1 | 0.00 | 0.00 | 0.00 | 0.00 | 0.00 | 0.00 | 0.00 | 0.00 | 0.00 | 0.00 | 0.00 | 0.00 |
| SIAH2 | 1.63 | 0.00 | 0.00 | 0.00 | 0.00 | 0.00 | 0.00 | 0.00 | 0.00 | 0.00 | 0.00 | 0.00 |
| SIGLECH | 0.00 | 0.00 | 0.00 | 0.00 | 0.00 | 0.00 | 0.00 | 0.00 | 0.00 | 0.00 | 0.00 | 2.56 |
| SIRPB1C | 0.00 | 0.00 | 0.00 | 0.00 | 0.00 | 0.00 | 0.00 | 0.00 | 0.00 | 0.00 | 0.00 | 0.00 |
| SLAMF9 | 0.00 | 1.42 | 0.00 | 0.00 | 0.00 | 0.00 | 0.00 | 0.00 | 0.00 | 0.00 | 0.00 | 0.00 |
| SLC7A11 | 0.00 | 4.20 | 0.00 | 0.00 | 0.00 | 0.00 | 0.00 | 0.00 | 2.25 | 0.00 | 0.00 | 9.12 |
| SLFN1 | 0.00 | 1.53 | 0.00 | 0.00 | 0.00 | 0.00 | 3.45 | 0.00 | 0.00 | 0.00 | 0.00 | 0.00 |
| SLFN2 | 0.00 | 1.63 | 0.00 | 0.00 | 0.00 | 2.50 | 2.04 | 0.00 | 1.44 | 0.00 | 0.00 | 0.00 |
| SLFN5 | 0.00 | 2.83 | 1.51 | 0.00 | 0.00 | 2.17 | 0.00 | 0.00 | 0.00 | 0.00 | 0.00 | 0.00 |
| SLFN8 | 0.00 | 0.00 | 0.00 | 0.00 | 0.00 | 0.00 | 0.00 | 0.00 | 0.00 | 0.00 | 0.00 | 0.00 |
| SLPI | 1.46 | 0.00 | 0.00 | 0.00 | 0.00 | 0.00 | 0.00 | 0.00 | 0.00 | 0.00 | 1.38 | 5.38 |
| SMIM14 | 0.00 | 0.00 | 0.00 | 0.00 | 0.00 | 0.00 | 0.00 | 0.00 | 0.00 | 0.00 | 0.00 | 0.00 |
| SMIM5 | 0.00 | 0.00 | 0.00 | 0.00 | 0.00 | 0.00 | 0.00 | 0.00 | 0.00 | 0.00 | 0.00 | 0.00 |
| SMOX | 0.00 | 0.00 | 0.00 | 0.00 | 0.00 | 0.00 | 0.00 | 0.00 | 0.00 | 0.00 | 0.00 | 3.28 |
| SNX9 | 1.63 | 0.00 | 0.00 | 0.00 | 0.00 | 0.00 | 0.00 | 0.00 | 0.00 | 0.00 | 0.00 | 0.00 |
| SOCS1 | 0.00 | 1.52 | 0.00 | 0.00 | 0.00 | 0.00 | 0.00 | 0.00 | 0.00 | 0.00 | 0.00 | 0.00 |
| SOCS3 | 1.50 | 0.00 | 0.00 | 0.00 | 0.00 | 0.00 | 0.00 | 0.00 | 0.00 | 0.00 | 0.00 | 0.00 |
| SP110 | 0.00 | 1.63 | 1.78 | 0.00 | 0.00 | 0.00 | 3.45 | 0.00 | 0.00 | 0.00 | 0.00 | 0.00 |
| SP140 | 0.00 | 1.40 | 0.00 | 0.00 | 0.00 | 0.00 | 0.00 | 0.00 | 0.00 | 0.00 | 0.00 | 0.00 |
| SPRED1 | 0.00 | 0.00 | 0.00 | 0.00 | 0.00 | 0.00 | 1.35 | 0.00 | 0.00 | 0.00 | 0.00 | 0.00 |
| SPRY2 | 0.00 | 0.00 | 0.00 | 0.00 | 0.00 | 0.00 | 0.00 | 0.00 | 0.00 | 0.00 | 0.00 | 3.09 |
| SPTY2D1 | 0.00 | 0.00 | 0.00 | 0.00 | 0.00 | 0.00 | 0.00 | 0.00 | 0.00 | 0.00 | 0.00 | 1.78 |
| SQLE | 0.00 | 0.00 | 1.47 | 0.00 | 0.00 | 0.00 | 0.00 | 0.00 | 0.00 | 0.00 | 0.00 | 0.00 |
| SRGN | 0.00 | 0.00 | 0.00 | 0.00 | 0.00 | 0.00 | 0.00 | 0.00 | 0.00 | 0.00 | 0.00 | 2.56 |
| SRSF5 | 2.41 | 0.00 | 0.00 | 0.00 | 0.00 | 0.00 | 0.00 | 0.00 | 0.00 | 0.00 | 0.00 | 0.00 |
| SSH2 | 0.00 | 0.00 | 0.00 | 0.00 | 0.00 | 0.00 | 0.00 | 0.00 | 0.00 | 0.00 | 0.00 | 0.00 |
| ST13 | 3.26 | 0.00 | 0.00 | 0.00 | 0.00 | 0.00 | 0.00 | 0.00 | 0.00 | 0.00 | 0.00 | 0.00 |
| ST3GAL6 | 0.00 | 0.00 | 0.00 | 0.00 | 0.00 | 0.00 | 0.00 | 0.00 | 0.00 | 0.00 | 0.00 | 3.97 |
| STAT1 | 0.00 | 0.00 | 1.48 | 0.00 | 0.00 | 0.00 | 0.00 | 0.00 | 0.00 | 0.00 | 0.00 | 0.00 |
| STAT2 | 0.00 | 2.47 | 0.00 | 0.00 | 0.00 | 0.00 | 0.00 | 0.00 | 2.34 | 0.00 | 0.00 | 0.00 |
| SV2C | 0.00 | 0.00 | 0.00 | 0.00 | 0.00 | 0.00 | 0.00 | 0.00 | 0.00 | 0.00 | 0.00 | 0.00 |
| SYK | 0.00 | 0.00 | 0.00 | 0.00 | 0.00 | 0.00 | 0.00 | 0.00 | 0.00 | 0.00 | 0.00 | 0.00 |
| SYNE1 | 0.00 | 0.00 | 0.00 | 0.00 | 0.00 | 0.00 | 0.00 | 0.00 | 0.00 | 0.00 | 0.00 | 1.65 |
| SYNGR1 | 0.00 | 0.00 | 0.00 | 0.00 | 0.00 | 0.00 | 0.00 | 0.00 | 0.00 | 0.00 | 0.00 | 0.00 |
| SYNJ1 | 0.00 | 1.98 | 0.00 | 0.00 | 0.00 | 0.00 | 0.00 | 0.00 | 0.00 | 0.00 | 0.00 | 0.00 |
| TAP1 | 0.00 | 1.63 | 0.00 | 0.00 | 0.00 | 0.00 | 0.00 | 0.00 | 0.00 | 0.00 | 0.00 | 0.00 |
| TAPBP | 0.00 | 0.00 | 0.00 | 0.00 | 0.00 | 0.00 | 0.00 | 0.00 | 1.63 | 0.00 | 0.00 | 0.00 |
| TBC1D8 | 0.00 | 0.00 | 0.00 | 0.00 | 0.00 | 0.00 | 0.00 | 0.00 | 0.00 | 0.00 | 0.00 | 0.00 |
| TCF4 | 0.00 | 0.00 | 0.00 | 0.00 | 0.00 | 0.00 | 0.00 | 0.00 | 0.00 | 0.00 | 0.00 | 0.00 |
| TESC | 0.00 | 0.00 | 0.00 | 0.00 | 0.00 | 0.00 | 0.00 | 0.00 | 0.00 | 0.00 | 0.00 | 0.00 |
| TGTP2 | 0.00 | 2.11 | 1.53 | 0.00 | 0.00 | 0.00 | 0.00 | 0.00 | 1.44 | 0.00 | 0.00 | 0.00 |
| THBS1 | 0.00 | 0.00 | 0.00 | 0.00 | 0.00 | 0.00 | 0.00 | 0.00 | 0.00 | 0.00 | 0.00 | 13.09 |
| THEMIS2 | 0.00 | 0.00 | 0.00 | 0.00 | 0.00 | 0.00 | 1.83 | 0.00 | 1.47 | 0.00 | 0.00 | 0.00 |
| TIFA | 0.00 | 0.00 | 0.00 | 0.00 | 0.00 | 0.00 | 0.00 | 0.00 | 0.00 | 0.00 | 0.00 | 0.00 |
| TIPARP | 0.00 | 0.00 | 0.00 | 0.00 | 0.00 | 0.00 | 0.00 | 0.00 | 0.00 | 0.00 | 0.00 | 2.42 |
| TLN1 | 0.00 | 0.00 | 0.00 | 0.00 | 0.00 | 0.00 | 0.00 | 0.00 | 0.00 | 0.00 | 0.00 | 0.00 |
| TMEM106A | 0.00 | 0.00 | 0.00 | 0.00 | 0.00 | 0.00 | 0.00 | 0.00 | 0.00 | 0.00 | 0.00 | 0.00 |
| TMEM106B | 0.00 | 0.00 | 0.00 | 0.00 | 0.00 | 0.00 | 0.00 | 0.00 | 0.00 | 0.00 | 0.00 | 0.00 |
| TMEM158 | 0.00 | 0.00 | 0.00 | 0.00 | 0.00 | 0.00 | 0.00 | 2.62 | 0.00 | 0.00 | 0.00 | 0.00 |
| TMEM176B | 0.00 | 2.61 | 0.00 | 0.00 | 0.00 | 0.00 | 0.00 | 0.00 | 0.00 | 0.00 | 0.00 | 0.00 |
| TMEM229B | 0.00 | 0.00 | 0.00 | 0.00 | 0.00 | 0.00 | 0.00 | 0.00 | 0.00 | 0.00 | 0.00 | 0.00 |
| TMEM55B | 0.00 | 0.00 | 0.00 | 0.00 | 0.00 | 0.00 | 0.00 | 0.00 | 0.00 | 0.00 | 0.00 | 0.00 |
| TNFAIP3 | 0.00 | 0.00 | 0.00 | 0.00 | 0.00 | 0.00 | 0.00 | 0.00 | 0.00 | 0.00 | 0.00 | 0.00 |
| TNFRSF13B | 1.33 | 0.00 | 0.00 | 0.00 | 0.00 | 0.00 | 0.00 | 0.00 | 0.00 | 0.00 | 0.00 | 0.00 |
| TNFRSF18 | 0.00 | 0.00 | 0.00 | 0.00 | 0.00 | 0.00 | 0.00 | 0.00 | 0.00 | 0.00 | 0.00 | 0.00 |
| TNFRSF25 | 0.00 | 0.00 | 0.00 | 0.00 | 0.00 | 0.00 | 0.00 | 0.00 | 0.00 | 0.00 | 0.00 | 0.00 |
| TNFRSF4 | 0.00 | 0.00 | 0.00 | 0.00 | 0.00 | 0.00 | 0.00 | 0.00 | 0.00 | 0.00 | 0.00 | 0.00 |
| TNFRSF9 | 0.00 | 1.99 | 0.00 | 0.00 | 0.00 | 0.00 | 0.00 | 2.19 | 0.00 | 0.00 | 0.00 | 0.00 |
| TNFSF10 | 0.00 | 1.63 | 0.00 | 0.00 | 0.00 | 0.00 | 0.00 | 0.00 | 0.00 | 0.00 | 0.00 | 0.00 |
| TNKS2 | 0.00 | 0.00 | 0.00 | 0.00 | 0.00 | 0.00 | 0.00 | 0.00 | 0.00 | 0.00 | 0.00 | 0.00 |
| TOB1 | 2.28 | 0.00 | 0.00 | 0.00 | 0.00 | 0.00 | 0.00 | 0.00 | 0.00 | 0.00 | 0.00 | 0.00 |
| TOR1AIP1 | 0.00 | 2.16 | 0.00 | 0.00 | 0.00 | 0.00 | 0.00 | 0.00 | 0.00 | 0.00 | 0.00 | 0.00 |
| TPI1 | 1.63 | 0.00 | 0.00 | 0.00 | 0.00 | 0.00 | 0.00 | 0.00 | 0.00 | 0.00 | 0.00 | 0.00 |
| TPM4 | 0.00 | 0.00 | 0.00 | 0.00 | 0.00 | 0.00 | 0.00 | 0.00 | 0.00 | 0.00 | 0.00 | 0.00 |
| TRA2B | 1.71 | 0.00 | 0.00 | 0.00 | 0.00 | 4.10 | 0.00 | 0.00 | 0.00 | 0.00 | 0.00 | 0.00 |
| TRAFD1 | 0.00 | 0.00 | 0.00 | 0.00 | 0.00 | 0.00 | 1.39 | 0.00 | 0.00 | 0.00 | 0.00 | 0.00 |
| TRAPPC10 | 0.00 | 0.00 | 0.00 | 0.00 | 0.00 | 0.00 | 0.00 | 0.00 | 0.00 | 0.00 | 0.00 | 0.00 |
| TREM1 | 0.00 | 0.00 | 0.00 | 0.00 | 0.00 | 0.00 | 0.00 | 0.00 | 0.00 | 0.00 | 0.00 | 2.73 |
| TRIB1 | 0.00 | 0.00 | 0.00 | 0.00 | 0.00 | 0.00 | 0.00 | 0.00 | 0.00 | 0.00 | 0.00 | 1.93 |
| TRIM14 | 0.00 | 1.53 | 0.00 | 0.00 | 0.00 | 0.00 | 0.00 | 0.00 | 0.00 | 0.00 | 0.00 | 0.00 |
| TRIM30A | 0.00 | 1.42 | 1.78 | 0.00 | 0.00 | 0.00 | 0.00 | 0.00 | 0.00 | 0.00 | 0.00 | 0.00 |
| TRIM30B | 0.00 | 0.00 | 0.00 | 0.00 | 0.00 | 0.00 | 2.04 | 0.00 | 0.00 | 0.00 | 0.00 | 0.00 |
| TRIM5 | 0.00 | 1.49 | 0.00 | 0.00 | 0.00 | 0.00 | 0.00 | 0.00 | 0.00 | 0.00 | 0.00 | 0.00 |
| TRIM56 | 0.00 | 0.00 | 0.00 | 0.00 | 0.00 | 0.00 | 0.00 | 0.00 | 0.00 | 0.00 | 0.00 | 0.00 |
| TRIP12 | 0.00 | 0.00 | 0.00 | 0.00 | 0.00 | 0.00 | 0.00 | 0.00 | 0.00 | 0.00 | 0.00 | 0.00 |
| TSC22D3 | 0.00 | 0.00 | 0.00 | 0.00 | 0.00 | 0.00 | 0.00 | 0.00 | 0.00 | 0.00 | 0.00 | 0.00 |
| TSIX | 0.00 | 0.00 | 0.00 | 0.00 | 0.00 | 0.00 | 0.00 | 0.00 | 0.00 | 0.00 | 0.00 | 0.00 |
| TTC37 | 0.00 | 0.00 | 0.00 | 0.00 | 0.00 | 0.00 | 0.00 | 0.00 | 0.00 | 0.00 | 0.00 | 0.00 |
| TUBA4A | 0.00 | 1.38 | 0.00 | 0.00 | 0.00 | 0.00 | 0.00 | 0.00 | 0.00 | 0.00 | 0.00 | 0.00 |
| TXK | 0.00 | 0.00 | 0.00 | 0.00 | 0.00 | 0.00 | 1.35 | 0.00 | 0.00 | 0.00 | 0.00 | 0.00 |
| TYRP1 | 0.00 | 0.00 | 0.00 | 0.00 | 0.00 | 2.47 | 0.00 | 0.00 | 0.00 | 0.00 | 0.00 | 0.00 |
| UBASH3B | 1.93 | 0.00 | 0.00 | 0.00 | 0.00 | 0.00 | 0.00 | 0.00 | 0.00 | 0.00 | 0.00 | 0.00 |
| UBB | 1.82 | 0.00 | 0.00 | 0.00 | 0.00 | 0.00 | 0.00 | 0.00 | 0.00 | 0.00 | 0.00 | 0.00 |
| UBC | 4.57 | 0.00 | 0.00 | 0.00 | 0.00 | 0.00 | 0.00 | 0.00 | 0.00 | 0.00 | 0.00 | 0.00 |
| UBE2L6 | 0.00 | 0.00 | 0.00 | 0.00 | 0.00 | 0.00 | 0.00 | 0.00 | 1.85 | 0.00 | 0.00 | 0.00 |
| UBE2S | 0.00 | 0.00 | 0.00 | 0.00 | 0.00 | 0.00 | 0.00 | 0.00 | 0.00 | 0.00 | 0.00 | 0.00 |
| UCK2 | 0.00 | 0.00 | 0.00 | 0.00 | 0.00 | 0.00 | 0.00 | 0.00 | 0.00 | 0.00 | 0.00 | 1.63 |
| UPP1 | 0.00 | 0.00 | 0.00 | 0.00 | 0.00 | 0.00 | 0.00 | 0.00 | 0.00 | 0.00 | 0.00 | 4.14 |
| USP12 | 0.00 | 0.00 | 0.00 | 0.00 | 0.00 | 0.00 | 2.04 | 0.00 | 0.00 | 0.00 | 0.00 | 0.00 |
| USP18 | 0.00 | 0.00 | 0.00 | 0.00 | 0.00 | 0.00 | 0.00 | 0.00 | 0.00 | 0.00 | 0.00 | 0.00 |
| VAV3 | 0.00 | 1.73 | 0.00 | 0.00 | 0.00 | 0.00 | 0.00 | 0.00 | 0.00 | 0.00 | 0.00 | 0.00 |
| VEGFA | 0.00 | 0.00 | 0.00 | 0.00 | 0.00 | 0.00 | 0.00 | 0.00 | 0.00 | 0.00 | 0.00 | 3.19 |
| VPS37B | 1.53 | 0.00 | 0.00 | 0.00 | 0.00 | 0.00 | 0.00 | 0.00 | 0.00 | 0.00 | 0.00 | 4.31 |
| WDR91 | 0.00 | 0.00 | 0.00 | 0.00 | 0.00 | 0.00 | 1.50 | 0.00 | 0.00 | 0.00 | 0.00 | 0.00 |
| WFDC17 | 0.00 | 0.00 | 0.00 | 0.00 | 0.00 | 0.00 | 0.00 | 0.00 | 0.00 | 0.00 | 0.00 | 3.52 |
| XAF1 | 0.00 | 0.00 | 0.00 | 0.00 | 0.00 | 0.00 | 2.42 | 0.00 | 0.00 | 0.00 | 0.00 | 0.00 |
| XPO6 | 0.00 | 0.00 | 0.00 | 0.00 | 0.00 | 1.41 | 0.00 | 0.00 | 0.00 | 0.00 | 0.00 | 0.00 |
| XPR1 | 0.00 | 1.86 | 0.00 | 0.00 | 0.00 | 0.00 | 0.00 | 0.00 | 0.00 | 0.00 | 0.00 | 0.00 |
| ZBP1 | 0.00 | 1.42 | 2.06 | 0.00 | 0.00 | 0.00 | 2.49 | 0.00 | 2.59 | 0.00 | 0.00 | 0.00 |
| ZFHX2 | 0.00 | 0.00 | 0.00 | 0.00 | 0.00 | 0.00 | 1.51 | 0.00 | 0.00 | 0.00 | 0.00 | 0.00 |
| ZFP106 | 0.00 | 1.99 | 0.00 | 0.00 | 0.00 | 0.00 | 1.52 | 0.00 | 0.00 | 0.00 | 0.00 | 0.00 |
| ZFP275 | 0.00 | 0.00 | 0.00 | 0.00 | 0.00 | 0.00 | 0.00 | 0.00 | 0.00 | 0.00 | 0.00 | 0.00 |
| ZFP36L2 | 3.50 | 0.00 | 0.00 | 0.00 | 0.00 | 0.00 | 0.00 | 0.00 | 0.00 | 0.00 | 0.00 | 0.00 |
| ZFP646 | 0.00 | 0.00 | 0.00 | 0.00 | 0.00 | 0.00 | 0.00 | 0.00 | 0.00 | 0.00 | 0.00 | 0.00 |
| ZFP931 | 0.00 | 0.00 | 0.00 | 0.00 | 0.00 | 0.00 | 0.00 | 0.00 | 0.00 | 0.00 | 0.00 | 0.00 |
| ZFP954 | 0.00 | 0.00 | 0.00 | 0.00 | 0.00 | 0.00 | 0.00 | 0.00 | 0.00 | 0.00 | 0.00 | 0.00 |
| ZFX | 0.00 | 1.68 | 0.00 | 0.00 | 0.00 | 0.00 | 0.00 | 0.00 | 0.00 | 0.00 | 0.00 | 0.00 |
| ZMYND15 | 0.00 | 0.00 | 1.43 | 0.00 | 0.00 | 0.00 | 0.00 | 0.00 | 0.00 | 0.00 | 0.00 | 0.00 |
| ZNFX1 | 0.00 | 2.47 | 0.00 | 0.00 | 0.00 | 0.00 | 0.00 | 0.00 | 0.00 | 0.00 | 0.00 | 0.00 |
| ZWINT | 0.00 | 0.00 | 0.00 | 0.00 | 0.00 | 0.00 | 1.82 | 0.00 | 0.00 | 0.00 | 0.00 | 0.00 |

TABLE 2B
T cell clusters

| Gene | T_0 | T_1 | T_2 | T_3 | T_4 | T_5 | T_6 | T_7 | T_8 | T_9 |
|---|---|---|---|---|---|---|---|---|---|---|
| KLRA7 | 0.00 | 0.00 | 0.00 | 0.00 | 0.00 | 0.00 | 0.00 | 4.29 | 0.00 | 0.00 |
| PENK | 0.00 | 0.00 | 0.00 | 0.00 | 3.08 | 0.00 | 0.00 | 3.43 | 0.00 | 0.00 |
| SPP1 | 0.00 | 0.00 | 0.00 | 0.00 | 0.00 | 0.00 | 0.00 | 3.43 | 0.00 | 0.00 |
| GM42418 | 4.92 | 4.09 | 2.77 | 3.41 | 2.17 | 15.95 | 5.22 | 3.31 | 2.94 | 4.40 |
| SAMD9L | 0.00 | 0.00 | 0.00 | 0.00 | 0.00 | 0.00 | 0.00 | 2.64 | 0.00 | 0.00 |
| ADRB2 | 2.88 | 0.00 | 0.00 | 1.36 | 0.00 | 0.00 | 0.00 | 2.39 | 0.00 | 0.00 |
| LGALS7 | 0.00 | 0.00 | 0.00 | 0.00 | 0.00 | 0.00 | 0.00 | 2.16 | 0.00 | 0.00 |
| SERPINE2 | 0.00 | 0.00 | 0.00 | 0.00 | 0.00 | 0.00 | 0.00 | 2.16 | 0.00 | 0.00 |
| TFF1 | 0.00 | 0.00 | 0.00 | 0.00 | 0.00 | 0.00 | 0.00 | 2.16 | 0.00 | 0.00 |
| CHKA | 0.00 | 0.00 | 1.65 | 0.00 | 0.00 | 0.00 | 0.00 | 1.99 | 0.00 | 0.00 |
| ATG7 | 0.00 | 0.00 | 0.00 | 0.00 | 0.00 | 1.74 | 0.00 | 1.98 | 0.00 | 0.00 |
| HIST1H2AP | 0.00 | 0.00 | 0.00 | 0.00 | 0.00 | 0.00 | 0.00 | 1.95 | 0.00 | 0.00 |
| S100A8 | 0.00 | 0.00 | 1.51 | 7.48 | 1.66 | 5.27 | 0.00 | 1.95 | 0.00 | 0.00 |
| IFIH1 | 0.00 | 0.00 | 0.00 | 0.00 | 0.00 | 0.00 | 0.00 | 1.81 | 0.00 | 0.00 |
| LGMN | 0.00 | 0.00 | 0.00 | 0.00 | 0.00 | 0.00 | 0.00 | 1.81 | 0.00 | 0.00 |
| MIR142HG | 0.00 | 0.00 | 0.00 | 0.00 | 0.00 | 0.00 | 0.00 | 1.81 | 0.00 | 0.00 |
| LILRB4A | 0.00 | 0.00 | 0.00 | 0.00 | 0.00 | 0.00 | 0.00 | 1.65 | 0.00 | 0.00 |
| AGAP2 | 0.00 | 0.00 | 0.00 | 0.00 | 0.00 | 0.00 | 0.00 | 1.59 | 0.00 | 0.00 |
| GM16586 | 0.00 | 0.00 | 0.00 | 0.00 | 0.00 | 0.00 | 0.00 | 1.58 | 0.00 | 0.00 |
| CCL8 | 0.00 | 1.32 | 0.00 | 1.73 | 0.00 | 0.00 | 0.00 | 1.50 | 0.00 | 0.00 |
| DCT | 0.00 | 0.00 | 0.00 | 0.00 | 0.00 | 0.00 | 0.00 | 1.50 | 0.00 | 0.00 |
| IFITM3 | 0.00 | 0.00 | 1.31 | 2.78 | 0.00 | 0.00 | 0.00 | 1.50 | 0.00 | 0.00 |
| C1QA | 0.00 | 0.00 | 0.00 | 2.84 | 0.00 | 0.00 | 0.00 | 1.47 | 0.00 | 0.00 |
| CTNND2 | 0.00 | 0.00 | 0.00 | 0.00 | 0.00 | 0.00 | 0.00 | 1.47 | 0.00 | 0.00 |
| PMS1 | 0.00 | 0.00 | 0.00 | 0.00 | 0.00 | 0.00 | 0.00 | 1.47 | 0.00 | 0.00 |
| TMEM209 | 0.00 | 0.00 | 0.00 | 0.00 | 0.00 | 0.00 | 0.00 | 1.47 | 0.00 | 0.00 |
| 1700025G04RIK | 0.00 | 0.00 | 0.00 | 2.05 | 0.00 | 0.00 | 0.00 | 0.00 | 0.00 | 0.00 |
| 5730455P16RIK | 0.00 | 0.00 | 0.00 | 0.00 | 0.00 | 0.00 | 0.00 | 0.00 | 0.00 | 0.00 |
| 9930111J21RIK2 | 1.32 | 2.59 | 0.00 | 0.00 | 0.00 | 0.00 | 0.00 | 0.00 | 0.00 | 0.00 |
| A130010J15RIK | 1.64 | 0.00 | 0.00 | 0.00 | 0.00 | 0.00 | 0.00 | 0.00 | 0.00 | 0.00 |
| A230046K03RIK | 0.00 | 0.00 | 0.00 | 1.33 | 0.00 | 0.00 | 0.00 | 0.00 | 0.00 | 0.00 |
| A330069E16RIK | 0.00 | 0.00 | 0.00 | 2.08 | 0.00 | 0.00 | 0.00 | 0.00 | 0.00 | 0.00 |
| A530040E14RIK | 0.00 | 0.00 | 0.00 | 0.00 | 0.00 | 0.00 | 0.00 | 0.00 | 0.00 | 0.00 |
| AA467197 | 0.00 | 0.00 | 0.00 | 0.00 | 0.00 | 0.00 | 0.00 | 0.00 | 0.00 | 0.00 |
| ABCC4 | 0.00 | 0.00 | 0.00 | 0.00 | 0.00 | 0.00 | 1.54 | 0.00 | 0.00 | 0.00 |
| AC125149.3 | 0.00 | 0.00 | 0.00 | 0.00 | 0.00 | 0.00 | 0.00 | 0.00 | 0.00 | 0.00 |
| ACADL | 0.00 | 0.00 | 0.00 | 0.00 | 0.00 | 0.00 | 0.00 | 0.00 | 0.00 | 0.00 |
| ACTB | 0.00 | 0.00 | 0.00 | 0.00 | 0.00 | 0.00 | 0.00 | 0.00 | 0.00 | 0.00 |
| ADAM8 | 0.00 | 0.00 | 0.00 | 0.00 | 0.00 | 0.00 | 0.00 | 0.00 | 0.00 | 0.00 |
| ADAP2 | 0.00 | 0.00 | 0.00 | 0.00 | 0.00 | 0.00 | 0.00 | 0.00 | 0.00 | 0.00 |
| ADAP2OS | 0.00 | 0.00 | 0.00 | 0.00 | 0.00 | 0.00 | 0.00 | 0.00 | 0.00 | 0.00 |
| ADPGK | 0.00 | 0.00 | 0.00 | 0.00 | 0.00 | 0.00 | 0.00 | 0.00 | 0.00 | 0.00 |
| AHCYL2 | 0.00 | 0.00 | 0.00 | 0.00 | 1.77 | 0.00 | 0.00 | 0.00 | 0.00 | 0.00 |
| AHNAK | 0.00 | 0.00 | 0.00 | 0.00 | 0.00 | 0.00 | 0.00 | 0.00 | 0.00 | 0.00 |
| AI607873 | 0.00 | 0.00 | 0.00 | 0.00 | 0.00 | 0.00 | 0.00 | 0.00 | 0.00 | 0.00 |
| AIF1 | 0.00 | 0.00 | 0.00 | 0.00 | 1.58 | 0.00 | 0.00 | 0.00 | 0.00 | 0.00 |
| AKAP2 | 0.00 | 0.00 | 0.00 | 0.00 | 0.00 | 0.00 | 0.00 | 0.00 | 0.00 | 0.00 |
| ANKRD33B | 0.00 | 0.00 | 0.00 | 0.00 | 0.00 | 0.00 | 0.00 | 0.00 | 0.00 | 0.00 |
| ANXA1 | 0.00 | 0.00 | 0.00 | 0.00 | 0.00 | 0.00 | 0.00 | 0.00 | 0.00 | 0.00 |
| APOD | 0.00 | 0.00 | 0.00 | 0.00 | 0.00 | 0.00 | 0.00 | 0.00 | 0.00 | 0.00 |
| APOE | 0.00 | 0.00 | 0.00 | 0.00 | 0.00 | 1.50 | 0.00 | 0.00 | 0.00 | 1.62 |
| APOL9B | 0.00 | 0.00 | 0.00 | 0.00 | 0.00 | 2.86 | 0.00 | 0.00 | 0.00 | 0.00 |
| AREG | 0.00 | 0.00 | 0.00 | 0.00 | 0.00 | 0.00 | 0.00 | 0.00 | 0.00 | 0.00 |
| ARG1 | 0.00 | 0.00 | 0.00 | 0.00 | 0.00 | 0.00 | 0.00 | 0.00 | 0.00 | 0.00 |
| ATAD2 | 0.00 | 0.00 | 0.00 | 0.00 | 1.77 | 0.00 | 0.00 | 0.00 | 0.00 | 0.00 |
| ATP2B1 | 0.00 | 0.00 | 0.00 | 0.00 | 0.00 | 1.64 | 0.00 | 0.00 | 0.00 | 0.00 |
| AVL9 | 0.00 | 0.00 | 0.00 | 1.66 | 0.00 | 0.00 | 0.00 | 0.00 | 0.00 | 0.00 |
| AY036118 | 6.16 | 6.16 | 2.77 | 4.26 | 0.00 | 8.51 | 7.21 | 0.00 | 1.71 | 2.05 |
| B3GALNT2 | 0.00 | 0.00 | 0.00 | 0.00 | 1.47 | 0.00 | 0.00 | 0.00 | 0.00 | 0.00 |
| BASP1 | 0.00 | 0.00 | 0.00 | 0.00 | 0.00 | 0.00 | 0.00 | 0.00 | 0.00 | 0.00 |
| BAZ1A | 0.00 | 0.00 | 1.78 | 0.00 | 0.00 | 0.00 | 0.00 | 0.00 | 0.00 | 0.00 |
| BC147527 | 0.00 | 0.00 | 0.00 | 0.00 | 0.00 | 0.00 | 0.00 | 0.00 | 0.00 | 0.00 |
| BCL11A | 0.00 | 0.00 | 0.00 | 0.00 | 0.00 | 0.00 | 0.00 | 0.00 | 1.41 | 0.00 |
| BCL2A1A | 0.00 | 0.00 | 0.00 | 0.00 | 0.00 | 0.00 | 0.00 | 0.00 | 0.00 | 0.00 |
| BCL2A1B | 0.00 | 0.00 | 0.00 | 0.00 | 0.00 | 0.00 | 0.00 | 0.00 | 0.00 | 0.00 |
| BHLHE40 | 0.00 | 0.00 | 1.47 | 0.00 | 0.00 | 0.00 | 0.00 | 0.00 | 0.00 | 0.00 |
| BNIP3 | 0.00 | 0.00 | 0.00 | 0.00 | 0.00 | 0.00 | 0.00 | 0.00 | 0.00 | 0.00 |
| BRPF1 | 0.00 | 0.00 | 0.00 | 1.94 | 0.00 | 0.00 | 0.00 | 0.00 | 0.00 | 0.00 |
| BST1 | 0.00 | 0.00 | 0.00 | 0.00 | 0.00 | 0.00 | 0.00 | 0.00 | 0.00 | 0.00 |
| BST2 | 0.00 | 0.00 | 1.65 | 0.00 | 0.00 | 0.00 | 0.00 | 0.00 | 0.00 | 0.00 |
| BTG3 | 0.00 | 0.00 | 0.00 | 0.00 | 1.79 | 0.00 | 0.00 | 0.00 | 0.00 | 0.00 |
| C130026I21RIK | 0.00 | 0.00 | 0.00 | 0.00 | 0.00 | 0.00 | 0.00 | 0.00 | 0.00 | 0.00 |
| C1QB | 1.41 | 0.00 | 0.00 | 1.51 | 0.00 | 0.00 | 0.00 | 0.00 | 0.00 | 0.00 |
| C1QC | 0.00 | 0.00 | 0.00 | 0.00 | 0.00 | 0.00 | 0.00 | 0.00 | 0.00 | 0.00 |
| C2CD4B | 0.00 | 0.00 | 0.00 | 0.00 | 0.00 | 0.00 | 0.00 | 0.00 | 0.00 | 0.00 |
| C3AR1 | 0.00 | 0.00 | 0.00 | 0.00 | 0.00 | 0.00 | 0.00 | 0.00 | 0.00 | 0.00 |
| CABLES1 | 0.00 | 0.00 | 0.00 | 0.00 | 0.00 | 0.00 | 0.00 | 0.00 | 0.00 | 0.00 |
| CACYBP | 0.00 | 0.00 | 0.00 | 0.00 | 0.00 | 0.00 | 0.00 | 0.00 | 0.00 | 0.00 |
| CALM1 | 0.00 | 0.00 | 0.00 | 0.00 | 0.00 | 0.00 | 0.00 | 0.00 | 0.00 | 0.00 |
| CARD19 | 0.00 | 0.00 | 0.00 | 0.00 | 0.00 | 0.00 | 0.00 | 0.00 | 0.00 | 0.00 |
| CCDC88C | 0.00 | 0.00 | 0.00 | 0.00 | 1.62 | 0.00 | 0.00 | 0.00 | 0.00 | 0.00 |
| CCL1 | 0.00 | 0.00 | 0.00 | 0.00 | 0.00 | 0.00 | 0.00 | 0.00 | 0.00 | 0.00 |
| CCL12 | 0.00 | 0.00 | 0.00 | 0.00 | 0.00 | 0.00 | 0.00 | 0.00 | 0.00 | 0.00 |
| CCL2 | 0.00 | 0.00 | 0.00 | 1.90 | 0.00 | 0.00 | 0.00 | 0.00 | 0.00 | 0.00 |
| CCL22 | 0.00 | 0.00 | 0.00 | 0.00 | 0.00 | 0.00 | 0.00 | 0.00 | 0.00 | 0.00 |
| CCL3 | 4.77 | 0.00 | 0.00 | 0.00 | 0.00 | 0.00 | 0.00 | 0.00 | 0.00 | 2.92 |
| CCL4 | 9.61 | 0.00 | 0.00 | 0.00 | 0.00 | 2.14 | 0.00 | 0.00 | 0.00 | 0.00 |
| CCL5 | 0.00 | 0.00 | 0.00 | 0.00 | 0.00 | 1.50 | 0.00 | 0.00 | 0.00 | 0.00 |
| CCL6 | 0.00 | 0.00 | 0.00 | 2.78 | 0.00 | 0.00 | 1.81 | 0.00 | 0.00 | 0.00 |
| CCL7 | 0.00 | 0.00 | 0.00 | 0.00 | 0.00 | 0.00 | 0.00 | 0.00 | 0.00 | 0.00 |
| CCNB1 | 0.00 | 0.00 | 0.00 | 0.00 | 0.00 | 0.00 | 0.00 | 0.00 | 0.00 | 0.00 |
| CCND1 | 0.00 | 0.00 | 0.00 | 0.00 | 0.00 | 0.00 | 0.00 | 0.00 | 0.00 | 0.00 |
| CCND2 | 0.00 | 0.00 | 0.00 | 0.00 | 0.00 | 0.00 | 0.00 | 0.00 | 0.00 | 0.00 |
| CCNL1 | 0.00 | 0.00 | 0.00 | 0.00 | 0.00 | 0.00 | 0.00 | 0.00 | 0.00 | 0.00 |
| CCNT2 | 0.00 | 0.00 | 0.00 | 0.00 | 0.00 | 0.00 | 0.00 | 0.00 | 0.00 | 0.00 |
| CCR1 | 0.00 | 0.00 | 0.00 | 0.00 | 0.00 | 0.00 | 0.00 | 0.00 | 0.00 | 0.00 |
| CCR2 | 0.00 | 0.00 | 0.00 | 0.00 | 0.00 | 0.00 | 0.00 | 0.00 | 0.00 | 1.44 |
| CCR3 | 0.00 | 0.00 | 2.12 | 0.00 | 0.00 | 1.47 | 0.00 | 0.00 | 0.00 | 0.00 |
| CCR5 | 0.00 | 0.00 | 0.00 | 0.00 | 0.00 | 0.00 | 0.00 | 0.00 | 0.00 | 0.00 |
| CCR9 | 0.00 | 1.51 | 0.00 | 0.00 | 0.00 | 0.00 | 0.00 | 0.00 | 0.00 | 0.00 |
| CCRL2 | 0.00 | 0.00 | 0.00 | 0.00 | 0.00 | 0.00 | 0.00 | 0.00 | 0.00 | 0.00 |
| CD14 | 0.00 | 0.00 | 0.00 | 0.00 | 0.00 | 0.00 | 0.00 | 0.00 | 0.00 | 0.00 |
| CD200 | 0.00 | 0.00 | 2.32 | 0.00 | 0.00 | 0.00 | 0.00 | 0.00 | 0.00 | 0.00 |
| CD209D | 0.00 | 0.00 | 0.00 | 0.00 | 0.00 | 0.00 | 0.00 | 0.00 | 4.42 | 0.00 |
| CD24A | 0.00 | 0.00 | 0.00 | 0.00 | 0.00 | 0.00 | 0.00 | 0.00 | 0.00 | 0.00 |
| CD28 | 2.77 | 1.72 | 0.00 | 0.00 | 0.00 | 0.00 | 0.00 | 0.00 | 0.00 | 0.00 |
| CD2AP | 0.00 | 0.00 | 0.00 | 0.00 | 0.00 | 0.00 | 0.00 | 0.00 | 0.00 | 0.00 |
| CD300C | 0.00 | 0.00 | 0.00 | 0.00 | 0.00 | 0.00 | 0.00 | 0.00 | 0.00 | 0.00 |
| CD302 | 1.37 | 0.00 | 0.00 | 0.00 | 0.00 | 0.00 | 0.00 | 0.00 | 0.00 | 0.00 |
| CD4 | 0.00 | 0.00 | 0.00 | 0.00 | 0.00 | 0.00 | 0.00 | 0.00 | 0.00 | 1.39 |
| CD40 | 0.00 | 0.00 | 0.00 | 0.00 | 0.00 | 0.00 | 0.00 | 0.00 | 0.00 | 0.00 |
| CD52 | 0.00 | 0.00 | 0.00 | 0.00 | 0.00 | 0.00 | 0.00 | 0.00 | 0.00 | 0.00 |
| CD63 | 0.00 | 0.00 | 0.00 | 0.00 | 0.00 | 0.00 | 0.00 | 0.00 | 0.00 | 0.00 |
| CD69 | 0.00 | 0.00 | 0.00 | 0.00 | 0.00 | 0.00 | 0.00 | 0.00 | 0.00 | 0.00 |
| CD72 | 0.00 | 0.00 | 0.00 | 0.00 | 0.00 | 0.00 | 0.00 | 0.00 | 0.00 | 0.00 |
| CD74 | 3.04 | 2.36 | 0.00 | 2.08 | 0.00 | 2.95 | 0.00 | 0.00 | 0.00 | 0.00 |
| CD79A | 0.00 | 0.00 | 0.00 | 0.00 | 0.00 | 0.00 | 0.00 | 0.00 | 0.00 | 0.00 |
| CD83 | 0.00 | 0.00 | 0.00 | 0.00 | 0.00 | 0.00 | 0.00 | 0.00 | 0.00 | 0.00 |
| CD8A | 0.00 | 0.00 | 0.00 | 0.00 | 0.00 | 0.00 | 0.00 | 0.00 | 0.00 | 1.94 |
| CD9 | 0.00 | 0.00 | 0.00 | 0.00 | 0.00 | 0.00 | 0.00 | 0.00 | 0.00 | 0.00 |
| CDK13 | 0.00 | 0.00 | 0.00 | 0.00 | 1.31 | 0.00 | 0.00 | 0.00 | 0.00 | 0.00 |
| CDK20 | 0.00 | 0.00 | 0.00 | 0.00 | 0.00 | 0.00 | 0.00 | 0.00 | 0.00 | 0.00 |
| CDKN2C | 0.00 | 0.00 | 0.00 | 0.00 | 0.00 | 0.00 | 0.00 | 0.00 | 0.00 | 0.00 |
| CEBPB | 2.47 | 0.00 | 0.00 | 1.63 | 0.00 | 0.00 | 0.00 | 0.00 | 0.00 | 0.00 |
| CEP170 | 0.00 | 0.00 | 0.00 | 0.00 | 1.52 | 0.00 | 0.00 | 0.00 | 0.00 | 0.00 |
| CEP250 | 0.00 | 0.00 | 0.00 | 0.00 | 1.43 | 0.00 | 0.00 | 0.00 | 0.00 | 0.00 |
| CFB | 0.00 | 0.00 | 0.00 | 0.00 | 0.00 | 0.00 | 0.00 | 0.00 | 0.00 | 0.00 |
| CITED2 | 2.29 | 0.00 | 0.00 | 0.00 | 0.00 | 0.00 | 0.00 | 0.00 | 0.00 | 0.00 |
| CKAP4 | 0.00 | 0.00 | 1.31 | 0.00 | 0.00 | 0.00 | 0.00 | 0.00 | 0.00 | 0.00 |
| CLEC4D | 0.00 | 0.00 | 0.00 | 0.00 | 0.00 | 0.00 | 0.00 | 0.00 | 0.00 | 0.00 |
| CLEC4E | 0.00 | 0.00 | 0.00 | 0.00 | 0.00 | 0.00 | 0.00 | 0.00 | 0.00 | 0.00 |
| CLEC4N | 0.00 | 0.00 | 0.00 | 0.00 | 0.00 | 0.00 | 0.00 | 0.00 | 0.00 | 0.00 |
| CLK1 | 2.32 | 0.00 | 0.00 | 0.00 | 0.00 | 0.00 | 0.00 | 0.00 | 0.00 | 0.00 |
| CMPK2 | 0.00 | 0.00 | 0.00 | 0.00 | 0.00 | 0.00 | 0.00 | 0.00 | 0.00 | 0.00 |
| COL4A3BP | 0.00 | 0.00 | 1.31 | 0.00 | 0.00 | 0.00 | 0.00 | 0.00 | 0.00 | 0.00 |
| COX6A2 | 0.00 | 0.00 | 0.00 | 0.00 | 0.00 | 0.00 | 0.00 | 0.00 | 4.42 | 0.00 |
| CREM | 0.00 | 0.00 | 0.00 | 0.00 | 0.00 | 0.00 | 0.00 | 0.00 | 0.00 | 0.00 |
| CROT | 1.37 | 0.00 | 0.00 | 0.00 | 0.00 | 0.00 | 0.00 | 0.00 | 0.00 | 0.00 |
| CSF1 | 0.00 | 1.53 | 0.00 | 0.00 | 0.00 | 0.00 | 0.00 | 0.00 | 0.00 | 0.00 |
| CSPRS | 0.00 | 0.00 | 0.00 | 0.00 | 0.00 | 0.00 | 0.00 | 0.00 | 0.00 | 0.00 |
| CSRNP1 | 0.00 | 0.00 | 0.00 | 0.00 | 0.00 | 0.00 | 0.00 | 0.00 | 0.00 | 0.00 |
| CSTB | 0.00 | 0.00 | 0.00 | 0.00 | 0.00 | 0.00 | 0.00 | 0.00 | 0.00 | 0.00 |
| CTLA4 | 0.00 | 0.00 | 0.00 | 0.00 | 0.00 | 0.00 | 0.00 | 0.00 | 0.00 | 4.55 |
| CTSD | 0.00 | 0.00 | 0.00 | 0.00 | 0.00 | 0.00 | 0.00 | 0.00 | 0.00 | 0.00 |
| CTSS | 0.00 | 0.00 | 0.00 | 0.00 | 0.00 | 0.00 | 0.00 | 0.00 | 0.00 | 0.00 |
| CXCL10 | 0.00 | 0.00 | 1.65 | 0.00 | 0.00 | 2.13 | 0.00 | 0.00 | 0.00 | 0.00 |
| CXCL2 | 6.46 | 0.00 | 0.00 | 2.18 | 0.00 | 0.00 | 0.00 | 0.00 | 0.00 | 0.00 |
| CXCL9 | 0.00 | 0.00 | 0.00 | 0.00 | 0.00 | 0.00 | 0.00 | 0.00 | 0.00 | 0.00 |
| CXCR4 | 0.00 | 0.00 | 0.00 | 0.00 | 0.00 | 0.00 | 0.00 | 0.00 | 0.00 | 0.00 |
| CXCR6 | 0.00 | 0.00 | 0.00 | 0.00 | 0.00 | 0.00 | 0.00 | 0.00 | 0.00 | 0.00 |
| CYBB | 0.00 | 0.00 | 0.00 | 0.00 | 0.00 | 0.00 | 0.00 | 0.00 | 2.63 | 0.00 |
| CYFIP1 | 0.00 | 0.00 | 0.00 | 0.00 | 0.00 | 0.00 | 0.00 | 0.00 | 0.00 | 0.00 |
| D13ERTD608E | 0.00 | 0.00 | 1.51 | 0.00 | 0.00 | 0.00 | 0.00 | 0.00 | 0.00 | 0.00 |
| DAB2 | 0.00 | 0.00 | 0.00 | 0.00 | 0.00 | 0.00 | 0.00 | 0.00 | 0.00 | 0.00 |
| DDIT3 | 0.00 | 0.00 | 0.00 | 0.00 | 0.00 | 0.00 | 0.00 | 0.00 | 0.00 | 0.00 |
| DDX5 | 0.00 | 0.00 | 0.00 | 1.55 | 0.00 | 0.00 | 0.00 | 0.00 | 0.00 | 0.00 |
| DDX58 | 1.60 | 0.00 | 0.00 | 0.00 | 0.00 | 0.00 | 0.00 | 0.00 | 0.00 | 0.00 |
| DDX60 | 0.00 | 0.00 | 0.00 | 0.00 | 0.00 | 0.00 | 0.00 | 0.00 | 0.00 | 0.00 |
| DGAT2 | 0.00 | 0.00 | 1.42 | 0.00 | 0.00 | 0.00 | 0.00 | 0.00 | 0.00 | 1.40 |
| DGCR8 | 0.00 | 0.00 | 1.57 | 0.00 | 0.00 | 0.00 | 0.00 | 0.00 | 0.00 | 0.00 |
| DGKG | 1.64 | 0.00 | 0.00 | 0.00 | 0.00 | 0.00 | 0.00 | 0.00 | 0.00 | 0.00 |
| DHX29 | 0.00 | 0.00 | 0.00 | 0.00 | 2.79 | 0.00 | 0.00 | 0.00 | 0.00 | 0.00 |
| DHX58 | 0.00 | 1.32 | 0.00 | 0.00 | 0.00 | 0.00 | 0.00 | 0.00 | 0.00 | 0.00 |
| DIP2B | 0.00 | 0.00 | 0.00 | 0.00 | 1.78 | 0.00 | 0.00 | 0.00 | 0.00 | 0.00 |
| DMKN | 0.00 | 0.00 | 0.00 | 0.00 | 0.00 | 0.00 | 0.00 | 0.00 | 0.00 | 0.00 |
| DNAJA1 | 0.00 | 0.00 | 0.00 | 0.00 | 0.00 | 0.00 | 0.00 | 0.00 | 0.00 | 0.00 |
| DNAJB1 | 4.60 | 0.00 | 1.78 | 5.95 | 0.00 | 2.16 | 0.00 | 0.00 | 0.00 | 0.00 |
| DNAJB4 | 0.00 | 0.00 | 1.42 | 0.00 | 0.00 | 0.00 | 0.00 | 0.00 | 0.00 | 0.00 |
| DNAJB9 | 0.00 | 0.00 | 0.00 | 0.00 | 0.00 | 0.00 | 0.00 | 0.00 | 0.00 | 0.00 |
| DNTTIP1 | 0.00 | 1.75 | 0.00 | 0.00 | 0.00 | 0.00 | 0.00 | 0.00 | 0.00 | 0.00 |
| DPYSL2 | 0.00 | 0.00 | 0.00 | 0.00 | 0.00 | 0.00 | 0.00 | 0.00 | 0.00 | 0.00 |
| DUSP1 | 0.00 | 0.00 | 0.00 | 2.41 | 0.00 | 1.64 | 0.00 | 0.00 | 0.00 | 0.00 |
| DUSP2 | 2.23 | 1.98 | 0.00 | 0.00 | 0.00 | 0.00 | 0.00 | 0.00 | 0.00 | 0.00 |
| EBF1 | 0.00 | 0.00 | 0.00 | 0.00 | 0.00 | 0.00 | 0.00 | 0.00 | 0.00 | 0.00 |
| EGLN3 | 0.00 | 0.00 | 0.00 | 0.00 | 0.00 | 0.00 | 0.00 | 0.00 | 0.00 | 0.00 |
| EGR1 | 0.00 | 0.00 | 0.00 | 0.00 | 0.00 | 0.00 | 0.00 | 0.00 | 0.00 | 0.00 |
| EIF5 | 0.00 | 0.00 | 0.00 | 0.00 | 0.00 | 0.00 | 0.00 | 0.00 | 0.00 | 0.00 |
| EML4 | 0.00 | 0.00 | 0.00 | 0.00 | 0.00 | 0.00 | 0.00 | 0.00 | 0.00 | 0.00 |
| ENPP2 | 0.00 | 1.46 | 0.00 | 0.00 | 0.00 | 0.00 | 0.00 | 0.00 | 0.00 | 0.00 |
| EOMES | 0.00 | 0.00 | 0.00 | 0.00 | 0.00 | 0.00 | 0.00 | 0.00 | 2.46 | 0.00 |
| ERDR1 | 3.69 | 2.61 | 2.68 | 6.47 | 0.00 | 2.09 | 0.00 | 0.00 | 0.00 | 0.00 |
| ERGIC2 | 0.00 | 0.00 | 0.00 | 0.00 | 0.00 | 0.00 | 0.00 | 0.00 | 0.00 | 0.00 |
| ERO1L | 0.00 | 0.00 | 0.00 | 0.00 | 0.00 | 0.00 | 0.00 | 0.00 | 0.00 | 0.00 |
| ETNK1 | 0.00 | 0.00 | 0.00 | 0.00 | 0.00 | 0.00 | 0.00 | 0.00 | 0.00 | 0.00 |
| ETS2 | 0.00 | 0.00 | 0.00 | 0.00 | 0.00 | 0.00 | 0.00 | 0.00 | 0.00 | 0.00 |
| ETV3 | 0.00 | 0.00 | 0.00 | 0.00 | 0.00 | 0.00 | 0.00 | 0.00 | 0.00 | 0.00 |
| FAM162A | 0.00 | 0.00 | 0.00 | 0.00 | 0.00 | 1.47 | 0.00 | 0.00 | 0.00 | 0.00 |
| FAM178A | 0.00 | 0.00 | 0.00 | 0.00 | 0.00 | 0.00 | 0.00 | 0.00 | 0.00 | 0.00 |
| FAM46A | 1.51 | 0.00 | 0.00 | 0.00 | 0.00 | 0.00 | 0.00 | 0.00 | 0.00 | 0.00 |
| FAM71A | 0.00 | 0.00 | 0.00 | 0.00 | 0.00 | 0.00 | 0.00 | 0.00 | 0.00 | 0.00 |
| FAM76B | 0.00 | 0.00 | 0.00 | 0.00 | 0.00 | 0.00 | 0.00 | 0.00 | 0.00 | 0.00 |
| FBLN5 | 0.00 | 0.00 | 0.00 | 0.00 | 0.00 | 0.00 | 0.00 | 0.00 | 0.00 | 0.00 |
| FBXW9 | 1.60 | 0.00 | 0.00 | 0.00 | 0.00 | 0.00 | 0.00 | 0.00 | 0.00 | 0.00 |
| FCER1G | 0.00 | 0.00 | 0.00 | 0.00 | 1.67 | 0.00 | 1.69 | 0.00 | 0.00 | 0.00 |
| FCGR1 | 0.00 | 0.00 | 0.00 | 0.00 | 0.00 | 0.00 | 2.36 | 0.00 | 0.00 | 0.00 |
| FCGR3 | 1.60 | 0.00 | 0.00 | 0.00 | 0.00 | 0.00 | 0.00 | 0.00 | 0.00 | 0.00 |
| FDFT1 | 0.00 | 0.00 | 0.00 | 0.00 | 0.00 | 0.00 | 0.00 | 0.00 | 0.00 | 0.00 |
| FILIP1L | 0.00 | 0.00 | 0.00 | 0.00 | 0.00 | 0.00 | 0.00 | 0.00 | 0.00 | 0.00 |
| FNDC3A | 0.00 | 0.00 | 0.00 | 0.00 | 0.00 | 0.00 | 0.00 | 0.00 | 0.00 | 0.00 |
| FOSB | 0.00 | 0.00 | 0.00 | 0.00 | 0.00 | 0.00 | 0.00 | 0.00 | 0.00 | 0.00 |
| FRMD4A | 0.00 | 0.00 | 0.00 | 0.00 | 0.00 | 0.00 | 0.00 | 0.00 | 0.00 | 0.00 |
| FRMD4B | 0.00 | 0.00 | 0.00 | 1.66 | 0.00 | 0.00 | 0.00 | 0.00 | 0.00 | 0.00 |
| FSCN1 | 0.00 | 0.00 | 0.00 | 0.00 | 0.00 | 0.00 | 0.00 | 0.00 | 0.00 | 0.00 |
| FTH1 | 0.00 | 0.00 | 0.00 | 0.00 | 0.00 | 0.00 | 0.00 | 0.00 | 0.00 | 0.00 |
| FTL1 | 0.00 | 0.00 | 0.00 | 0.00 | 0.00 | 0.00 | 0.00 | 0.00 | 0.00 | 0.00 |
| G0S2 | 0.00 | 0.00 | 0.00 | 0.00 | 0.00 | 0.00 | 0.00 | 0.00 | 0.00 | 0.00 |
| GADD45B | 0.00 | 0.00 | 0.00 | 0.00 | 0.00 | 0.00 | 0.00 | 0.00 | 0.00 | 0.00 |
| GADD45G | 0.00 | 0.00 | 0.00 | 16.45 | 0.00 | 0.00 | 0.00 | 0.00 | 0.00 | 0.00 |
| GAS2 | 0.00 | 0.00 | 1.78 | 0.00 | 0.00 | 0.00 | 0.00 | 0.00 | 0.00 | 0.00 |
| GBP3 | 0.00 | 0.00 | 1.32 | 0.00 | 0.00 | 0.00 | 0.00 | 0.00 | 0.00 | 0.00 |
| GBP4 | 0.00 | 0.00 | 0.00 | 0.00 | 0.00 | 0.00 | 0.00 | 0.00 | 0.00 | 0.00 |
| GBP5 | 0.00 | 0.00 | 0.00 | 0.00 | 0.00 | 0.00 | 0.00 | 0.00 | 0.00 | 0.00 |
| GBP9 | 0.00 | 0.00 | 1.31 | 0.00 | 0.00 | 0.00 | 0.00 | 0.00 | 0.00 | 0.00 |
| GCNT2 | 0.00 | 0.00 | 0.00 | 0.00 | 0.00 | 0.00 | 0.00 | 0.00 | 0.00 | 0.00 |
| GDPGP1 | 0.00 | 0.00 | 0.00 | 0.00 | 1.78 | 0.00 | 0.00 | 0.00 | 0.00 | 0.00 |
| GEM | 0.00 | 0.00 | 0.00 | 2.37 | 0.00 | 0.00 | 0.00 | 0.00 | 0.00 | 0.00 |
| GGA3 | 1.60 | 0.00 | 0.00 | 0.00 | 0.00 | 0.00 | 0.00 | 0.00 | 0.00 | 0.00 |
| GIMAP9 | 0.00 | 0.00 | 0.00 | 1.68 | 0.00 | 0.00 | 0.00 | 0.00 | 0.00 | 0.00 |
| GINS2 | 0.00 | 0.00 | 0.00 | 0.00 | 0.00 | 0.00 | 0.00 | 0.00 | 0.00 | 0.00 |
| GM10076 | 0.00 | 2.11 | 0.00 | 3.32 | 0.00 | 0.00 | 0.00 | 0.00 | 0.00 | 0.00 |
| GM10116 | 0.00 | 0.00 | 0.00 | 0.00 | 0.00 | 0.00 | 0.00 | 0.00 | 0.00 | 0.00 |
| GM10263 | 0.00 | 0.00 | 0.00 | 1.67 | 0.00 | 0.00 | 0.00 | 0.00 | 0.00 | 0.00 |
| GM10269 | 0.00 | 1.53 | 0.00 | 2.02 | 0.00 | 0.00 | 0.00 | 0.00 | 0.00 | 0.00 |
| GM11175 | 0.00 | 0.00 | 0.00 | 0.00 | 0.00 | 0.00 | 0.00 | 0.00 | 0.00 | 0.00 |
| GM11808 | 0.00 | 0.00 | 0.00 | 0.00 | 0.00 | 0.00 | 0.00 | 0.00 | 0.00 | 1.44 |
| GM12185 | 0.00 | 0.00 | 0.00 | 1.58 | 0.00 | 0.00 | 0.00 | 0.00 | 0.00 | 0.00 |
| GM12216 | 0.00 | 0.00 | 0.00 | 0.00 | 0.00 | 0.00 | 0.00 | 0.00 | 0.00 | 0.00 |
| GM17056 | 0.00 | 0.00 | 0.00 | 0.00 | 0.00 | 0.00 | 0.00 | 0.00 | 0.00 | 0.00 |
| GM2000 | 3.26 | 1.69 | 0.00 | 2.42 | 0.00 | 0.00 | 0.00 | 0.00 | 0.00 | 1.44 |
| GM26518 | 1.91 | 0.00 | 0.00 | 0.00 | 0.00 | 0.00 | 0.00 | 0.00 | 0.00 | 0.00 |
| GM26522 | 0.00 | 0.00 | 0.00 | 0.00 | 0.00 | 0.00 | 0.00 | 0.00 | 0.00 | 0.00 |
| GM26532 | 0.00 | 0.00 | 0.00 | 0.00 | 0.00 | 0.00 | 0.00 | 0.00 | 0.00 | 0.00 |
| GM26541 | 0.00 | 0.00 | 0.00 | 0.00 | 2.11 | 0.00 | 0.00 | 0.00 | 0.00 | 0.00 |
| GM26545 | 1.73 | 0.00 | 0.00 | 0.00 | 0.00 | 2.39 | 0.00 | 0.00 | 0.00 | 0.00 |
| GM26699 | 3.26 | 0.00 | 0.00 | 1.67 | 0.00 | 0.00 | 0.00 | 0.00 | 0.00 | 0.00 |
| GM26917 | 1.52 | 0.00 | 0.00 | 0.00 | 0.00 | 1.74 | 0.00 | 0.00 | 0.00 | 0.00 |
| GM4070 | 1.71 | 0.00 | 1.40 | 0.00 | 0.00 | 0.00 | 0.00 | 0.00 | 0.00 | 0.00 |
| GM43603 | 2.33 | 0.00 | 0.00 | 0.00 | 0.00 | 0.00 | 0.00 | 0.00 | 0.00 | 0.00 |
| GM4955 | 0.00 | 1.82 | 1.44 | 0.00 | 0.00 | 0.00 | 0.00 | 0.00 | 0.00 | 0.00 |
| GM5093 | 0.00 | 0.00 | 0.00 | 1.51 | 0.00 | 0.00 | 0.00 | 0.00 | 0.00 | 0.00 |
| GM8186 | 0.00 | 0.00 | 1.44 | 0.00 | 0.00 | 0.00 | 0.00 | 0.00 | 0.00 | 0.00 |
| GM8797 | 0.00 | 0.00 | 0.00 | 1.71 | 0.00 | 0.00 | 0.00 | 0.00 | 0.00 | 0.00 |
| GM8953 | 0.00 | 0.00 | 0.00 | 0.00 | 0.00 | 0.00 | 0.00 | 0.00 | 0.00 | 0.00 |
| GPD2 | 0.00 | 0.00 | 0.00 | 0.00 | 0.00 | 0.00 | 0.00 | 0.00 | 0.00 | 0.00 |
| GPNMB | 0.00 | 0.00 | 0.00 | 0.00 | 0.00 | 0.00 | 0.00 | 0.00 | 0.00 | 0.00 |
| GRB2 | 0.00 | 0.00 | 0.00 | 0.00 | 0.00 | 0.00 | 0.00 | 0.00 | 0.00 | 0.00 |
| GRINA | 0.00 | 0.00 | 0.00 | 0.00 | 0.00 | 0.00 | 0.00 | 0.00 | 0.00 | 0.00 |
| GSR | 0.00 | 0.00 | 0.00 | 0.00 | 0.00 | 0.00 | 0.00 | 0.00 | 0.00 | 0.00 |
| GZMA | 0.00 | 0.00 | 0.00 | 3.02 | 2.21 | 0.00 | 0.00 | 0.00 | 0.00 | 0.00 |
| GZMB | 0.00 | 0.00 | 0.00 | 0.00 | 0.00 | 4.71 | 0.00 | 0.00 | 0.00 | 1.32 |
| H2-AA | 3.89 | 1.69 | 0.00 | 0.00 | 0.00 | 0.00 | 0.00 | 0.00 | 0.00 | 0.00 |
| H2-AB1 | 6.16 | 1.72 | 0.00 | 0.00 | 2.17 | 2.47 | 0.00 | 0.00 | 0.00 | 0.00 |
| H2-EB1 | 3.06 | 2.11 | 0.00 | 0.00 | 1.77 | 1.67 | 0.00 | 0.00 | 0.00 | 0.00 |
| H2-M3 | 0.00 | 0.00 | 0.00 | 0.00 | 0.00 | 0.00 | 0.00 | 0.00 | 0.00 | 0.00 |
| HBA-A1 | 0.00 | 15.81 | 0.00 | 0.00 | 0.00 | 0.00 | 0.00 | 0.00 | 0.00 | 0.00 |
| HBA-A2 | 0.00 | 19.75 | 0.00 | 0.00 | 0.00 | 0.00 | 0.00 | 0.00 | 0.00 | 0.00 |
| HBB-BS | 0.00 | 6.53 | 0.00 | 0.00 | 0.00 | 0.00 | 0.00 | 0.00 | 0.00 | 0.00 |
| HBB-BT | 0.00 | 27.42 | 0.00 | 0.00 | 0.00 | 0.00 | 0.00 | 0.00 | 0.00 | 0.00 |
| HCAR2 | 0.00 | 0.00 | 0.00 | 0.00 | 0.00 | 0.00 | 0.00 | 0.00 | 0.00 | 0.00 |
| HDC | 0.00 | 0.00 | 0.00 | 0.00 | 0.00 | 0.00 | 0.00 | 0.00 | 0.00 | 0.00 |
| HDLBP | 0.00 | 0.00 | 0.00 | 0.00 | 1.99 | 0.00 | 0.00 | 0.00 | 0.00 | 0.00 |
| HERC6 | 0.00 | 0.00 | 0.00 | 0.00 | 0.00 | 0.00 | 0.00 | 0.00 | 0.00 | 0.00 |
| HES1 | 0.00 | 0.00 | 0.00 | 0.00 | 0.00 | 0.00 | 0.00 | 0.00 | 0.00 | 2.84 |
| HILPDA | 0.00 | 0.00 | 0.00 | 0.00 | 0.00 | 1.50 | 0.00 | 0.00 | 0.00 | 0.00 |
| HIST1H1B | 0.00 | 0.00 | 0.00 | 0.00 | 0.00 | 0.00 | 0.00 | 0.00 | 2.11 | 0.00 |
| HIST1H1C | 0.00 | 0.00 | 0.00 | 1.31 | 0.00 | 0.00 | 0.00 | 0.00 | 0.00 | 0.00 |
| HIST1H1E | 0.00 | 0.00 | 0.00 | 0.00 | 0.00 | 0.00 | 0.00 | 0.00 | 1.70 | 0.00 |
| HIST1H2BC | 0.00 | 0.00 | 0.00 | 0.00 | 0.00 | 0.00 | 0.00 | 0.00 | 0.00 | 0.00 |
| HIST1H2BJ | 0.00 | 0.00 | 0.00 | 0.00 | 3.35 | 0.00 | 0.00 | 0.00 | 0.00 | 0.00 |
| HIST1H4H | 0.00 | 1.32 | 0.00 | 0.00 | 0.00 | 0.00 | 0.00 | 0.00 | 0.00 | 0.00 |
| HIST2H2AA1 | 0.00 | 0.00 | 0.00 | 0.00 | 0.00 | 0.00 | 0.00 | 0.00 | 0.00 | 0.00 |
| HMOX1 | 0.00 | 0.00 | 0.00 | 0.00 | 0.00 | 0.00 | 0.00 | 0.00 | 0.00 | 0.00 |
| HPGDS | 0.00 | 0.00 | 1.78 | 0.00 | 0.00 | 0.00 | 0.00 | 0.00 | 0.00 | 0.00 |
| HSP90AA1 | 2.68 | 0.00 | 0.00 | 1.93 | 0.00 | 0.00 | 0.00 | 0.00 | 0.00 | 0.00 |
| HSP90AB1 | 0.00 | 0.00 | 0.00 | 0.00 | 0.00 | 0.00 | 0.00 | 0.00 | 0.00 | 0.00 |
| HSPA1A | 0.00 | 0.00 | 0.00 | 26.34 | 0.00 | 1.34 | 0.00 | 0.00 | 10.39 | 1.40 |
| HSPA1B | 0.00 | 0.00 | 0.00 | 5.95 | 0.00 | 0.00 | 0.00 | 0.00 | 0.00 | 0.00 |
| HSPA8 | 0.00 | 0.00 | 0.00 | 0.00 | 0.00 | 0.00 | 0.00 | 0.00 | 0.00 | 0.00 |
| HSPD1 | 0.00 | 1.31 | 0.00 | 0.00 | 0.00 | 0.00 | 0.00 | 0.00 | 0.00 | 0.00 |
| HSPE1 | 0.00 | 0.00 | 0.00 | 0.00 | 0.00 | 0.00 | 0.00 | 0.00 | 0.00 | 0.00 |
| HTR7 | 0.00 | 0.00 | 0.00 | 0.00 | 0.00 | 0.00 | 0.00 | 0.00 | 0.00 | 0.00 |
| ICAM1 | 0.00 | 0.00 | 0.00 | 0.00 | 1.80 | 0.00 | 0.00 | 0.00 | 0.00 | 0.00 |
| ICE1 | 0.00 | 0.00 | 1.44 | 0.00 | 0.00 | 0.00 | 0.00 | 0.00 | 0.00 | 0.00 |
| ICOS | 0.00 | 0.00 | 0.00 | 0.00 | 0.00 | 0.00 | 0.00 | 0.00 | 0.00 | 1.42 |
| ID1 | 0.00 | 0.00 | 0.00 | 0.00 | 0.00 | 0.00 | 0.00 | 0.00 | 0.00 | 0.00 |
| ID2 | 0.00 | 0.00 | 0.00 | 2.79 | 0.00 | 2.13 | 0.00 | 0.00 | 0.00 | 0.00 |
| ID3 | 0.00 | 0.00 | 0.00 | 1.85 | 2.17 | 0.00 | 0.00 | 0.00 | 0.00 | 0.00 |
| IER2 | 2.42 | 0.00 | 0.00 | 4.26 | 0.00 | 0.00 | 0.00 | 0.00 | 0.00 | 0.00 |
| IER3 | 0.00 | 0.00 | 0.00 | 0.00 | 0.00 | 0.00 | 0.00 | 0.00 | 0.00 | 0.00 |
| IFI203 | 1.89 | 0.00 | 0.00 | 0.00 | 0.00 | 0.00 | 0.00 | 0.00 | 0.00 | 0.00 |
| IFI204 | 0.00 | 0.00 | 0.00 | 0.00 | 0.00 | 0.00 | 0.00 | 0.00 | 0.00 | 0.00 |
| IFI205 | 0.00 | 0.00 | 0.00 | 0.00 | 0.00 | 0.00 | 0.00 | 0.00 | 0.00 | 0.00 |
| IFI27L2A | 4.80 | 0.00 | 3.43 | 0.00 | 0.00 | 4.98 | 0.00 | 0.00 | 3.07 | 3.15 |
| IFI35 | 0.00 | 0.00 | 0.00 | 0.00 | 0.00 | 0.00 | 0.00 | 0.00 | 0.00 | 0.00 |
| IFI47 | 0.00 | 0.00 | 2.68 | 0.00 | 0.00 | 0.00 | 0.00 | 0.00 | 0.00 | 0.00 |
| IFIT1 | 2.59 | 3.09 | 0.00 | 0.00 | 0.00 | 4.98 | 0.00 | 0.00 | 0.00 | 0.00 |
| IFIT1BL1 | 0.00 | 0.00 | 0.00 | 0.00 | 0.00 | 2.79 | 0.00 | 0.00 | 0.00 | 0.00 |
| IFIT3 | 1.89 | 3.17 | 0.00 | 1.58 | 0.00 | 2.94 | 0.00 | 0.00 | 0.00 | 0.00 |
| IFIT3B | 1.68 | 0.00 | 0.00 | 0.00 | 0.00 | 0.00 | 0.00 | 0.00 | 0.00 | 0.00 |
| IFITM1 | 0.00 | 1.32 | 0.00 | 0.00 | 0.00 | 0.00 | 0.00 | 0.00 | 0.00 | 0.00 |
| IFNGR1 | 0.00 | 0.00 | 0.00 | 0.00 | 0.00 | 0.00 | 0.00 | 0.00 | 0.00 | 1.44 |
| IFRD1 | 0.00 | 0.00 | 0.00 | 0.00 | 0.00 | 0.00 | 0.00 | 0.00 | 0.00 | 0.00 |
| IFT22 | 0.00 | 0.00 | 0.00 | 0.00 | 0.00 | 0.00 | 0.00 | 0.00 | 0.00 | 0.00 |
| IGTP | 0.00 | 0.00 | 1.71 | 0.00 | 0.00 | 1.31 | 0.00 | 0.00 | 0.00 | 0.00 |
| IIGP1 | 0.00 | 0.00 | 0.00 | 0.00 | 0.00 | 1.86 | 0.00 | 0.00 | 0.00 | 0.00 |
| IL18BP | 0.00 | 0.00 | 0.00 | 0.00 | 0.00 | 0.00 | 0.00 | 0.00 | 0.00 | 0.00 |
| IL1B | 0.00 | 0.00 | 0.00 | 0.00 | 0.00 | 0.00 | 0.00 | 0.00 | 0.00 | 0.00 |
| IL1R2 | 0.00 | 0.00 | 0.00 | 0.00 | 0.00 | 0.00 | 0.00 | 0.00 | 0.00 | 0.00 |
| IL1RL1 | 0.00 | 0.00 | 2.10 | 0.00 | 0.00 | 0.00 | 0.00 | 0.00 | 0.00 | 0.00 |
| IL1RN | 0.00 | 0.00 | 0.00 | 0.00 | 0.00 | 0.00 | 0.00 | 0.00 | 0.00 | 0.00 |
| IL4I1 | 0.00 | 0.00 | 0.00 | 0.00 | 0.00 | 0.00 | 0.00 | 0.00 | 0.00 | 1.35 |
| INTS5 | 0.00 | 0.00 | 0.00 | 0.00 | 0.00 | 0.00 | 0.00 | 0.00 | 0.00 | 0.00 |
| IQGAP2 | 0.00 | 0.00 | 0.00 | 0.00 | 2.04 | 0.00 | 0.00 | 0.00 | 0.00 | 0.00 |
| IRF7 | 0.00 | 0.00 | 1.97 | 0.00 | 0.00 | 2.35 | 0.00 | 0.00 | 0.00 | 0.00 |
| IRG1 | 0.00 | 0.00 | 0.00 | 0.00 | 0.00 | 0.00 | 0.00 | 0.00 | 0.00 | 0.00 |
| ISG15 | 3.52 | 2.80 | 2.11 | 1.67 | 0.00 | 3.58 | 0.00 | 0.00 | 0.00 | 0.00 |
| ISG20 | 0.00 | 0.00 | 0.00 | 0.00 | 0.00 | 0.00 | 0.00 | 0.00 | 0.00 | 0.00 |
| ITGA4 | 0.00 | 0.00 | 0.00 | 0.00 | 0.00 | 0.00 | 0.00 | 0.00 | 0.00 | 0.00 |
| ITGB7 | 0.00 | 0.00 | 0.00 | 0.00 | 0.00 | 0.00 | 0.00 | 0.00 | 0.00 | 1.62 |
| JADE1 | 0.00 | 0.00 | 0.00 | 0.00 | 1.45 | 0.00 | 0.00 | 0.00 | 0.00 | 0.00 |
| JUN | 3.69 | 0.00 | 2.10 | 8.70 | 0.00 | 2.39 | 0.00 | 0.00 | 0.00 | 1.55 |
| JUNB | 4.26 | 2.71 | 0.00 | 8.58 | 0.00 | 0.00 | 0.00 | 0.00 | 0.00 | 0.00 |
| KDM6B | 0.00 | 0.00 | 0.00 | 0.00 | 0.00 | 0.00 | 0.00 | 0.00 | 0.00 | 0.00 |
| KDR | 0.00 | 0.00 | 0.00 | 0.00 | 0.00 | 0.00 | 0.00 | 0.00 | 0.00 | 0.00 |
| KIFC1 | 0.00 | 0.00 | 2.36 | 0.00 | 0.00 | 0.00 | 0.00 | 0.00 | 0.00 | 0.00 |
| KIT | 0.00 | 0.00 | 0.00 | 0.00 | 0.00 | 0.00 | 0.00 | 0.00 | 0.00 | 0.00 |
| KLF2 | 2.48 | 0.00 | 0.00 | 0.00 | 0.00 | 0.00 | 0.00 | 0.00 | 0.00 | 0.00 |
| KLF3 | 0.00 | 0.00 | 0.00 | 0.00 | 0.00 | 0.00 | 0.00 | 0.00 | 1.70 | 0.00 |
| KLF9 | 0.00 | 0.00 | 0.00 | 0.00 | 0.00 | 0.00 | 0.00 | 0.00 | 0.00 | 0.00 |
| KLRA1 | 0.00 | 0.00 | 0.00 | 0.00 | 0.00 | 0.00 | 0.00 | 0.00 | 0.00 | 4.55 |
| KLRA8 | 0.00 | 0.00 | 0.00 | 0.00 | 0.00 | 0.00 | 0.00 | 0.00 | 0.00 | 1.44 |
| KLRA9 | 0.00 | 0.00 | 0.00 | 0.00 | 1.94 | 0.00 | 0.00 | 0.00 | 0.00 | 0.00 |
| KLRB1B | 0.00 | 0.00 | 0.00 | 0.00 | 0.00 | 0.00 | 0.00 | 0.00 | 0.00 | 0.00 |
| KLRC1 | 0.00 | 0.00 | 0.00 | 0.00 | 0.00 | 1.74 | 0.00 | 0.00 | 0.00 | 0.00 |
| KLRD1 | 0.00 | 0.00 | 2.18 | 0.00 | 0.00 | 0.00 | 0.00 | 0.00 | 0.00 | 0.00 |
| KLRG1 | 0.00 | 0.00 | 0.00 | 0.00 | 0.00 | 0.00 | 0.00 | 0.00 | 0.00 | 0.00 |
| KLRK1 | 0.00 | 0.00 | 0.00 | 0.00 | 0.00 | 0.00 | 0.00 | 0.00 | 0.00 | 0.00 |
| KMO | 0.00 | 0.00 | 0.00 | 0.00 | 0.00 | 0.00 | 0.00 | 0.00 | 0.00 | 0.00 |
| KPNA2 | 0.00 | 0.00 | 0.00 | 0.00 | 1.46 | 0.00 | 0.00 | 0.00 | 0.00 | 0.00 |
| LDHA | 0.00 | 0.00 | 0.00 | 0.00 | 0.00 | 0.00 | 0.00 | 0.00 | 0.00 | 0.00 |
| LEPR | 0.00 | 0.00 | 0.00 | 0.00 | 0.00 | 0.00 | 0.00 | 0.00 | 0.00 | 0.00 |
| LFNG | 0.00 | 0.00 | 0.00 | 0.00 | 0.00 | 0.00 | 0.00 | 0.00 | 0.00 | 1.49 |
| LGALS3BP | 0.00 | 0.00 | 0.00 | 0.00 | 0.00 | 0.00 | 0.00 | 0.00 | 0.00 | 0.00 |
| LILR4B | 0.00 | 0.00 | 0.00 | 0.00 | 0.00 | 0.00 | 0.00 | 0.00 | 0.00 | 0.00 |
| LIPA | 0.00 | 0.00 | 0.00 | 0.00 | 1.45 | 0.00 | 0.00 | 0.00 | 0.00 | 0.00 |
| LITAF | 0.00 | 0.00 | 0.00 | 0.00 | 0.00 | 0.00 | 0.00 | 0.00 | 0.00 | 0.00 |
| LMBRD2 | 0.00 | 0.00 | 0.00 | 0.00 | 2.24 | 0.00 | 0.00 | 0.00 | 0.00 | 0.00 |
| LMNB1 | 0.00 | 0.00 | 0.00 | 0.00 | 0.00 | 0.00 | 0.00 | 0.00 | 0.00 | 0.00 |
| LNPEP | 0.00 | 0.00 | 0.00 | 0.00 | 1.80 | 0.00 | 0.00 | 0.00 | 0.00 | 0.00 |
| LRG1 | 0.00 | 0.00 | 0.00 | 0.00 | 0.00 | 0.00 | 0.00 | 0.00 | 0.00 | 0.00 |
| LRRC25 | 0.00 | 0.00 | 0.00 | 0.00 | 0.00 | 0.00 | 0.00 | 0.00 | 0.00 | 0.00 |
| LTA | 0.00 | 0.00 | 0.00 | 0.00 | 0.00 | 0.00 | 0.00 | 0.00 | 0.00 | 0.00 |
| LTB4R1 | 1.36 | 0.00 | 0.00 | 0.00 | 0.00 | 0.00 | 0.00 | 0.00 | 0.00 | 0.00 |
| LY6A | 0.00 | 0.00 | 0.00 | 1.52 | 0.00 | 0.00 | 0.00 | 0.00 | 0.00 | 3.15 |
| LY6C2 | 1.37 | 0.00 | 0.00 | 1.74 | 0.00 | 0.00 | 0.00 | 0.00 | 0.00 | 1.44 |
| LY6D | 0.00 | 0.00 | 0.00 | 0.00 | 0.00 | 0.00 | 0.00 | 0.00 | 0.00 | 0.00 |
| LY6E | 1.41 | 0.00 | 0.00 | 0.00 | 0.00 | 0.00 | 0.00 | 0.00 | 0.00 | 0.00 |
| LYZ2 | 1.41 | 3.80 | 0.00 | 4.86 | 0.00 | 5.01 | 1.31 | 0.00 | 0.00 | 0.00 |
| MACROD1 | 0.00 | 0.00 | 0.00 | 0.00 | 1.58 | 0.00 | 0.00 | 0.00 | 0.00 | 0.00 |
| MAFF | 0.00 | 0.00 | 0.00 | 0.00 | 0.00 | 0.00 | 0.00 | 0.00 | 0.00 | 0.00 |
| MAP3K8 | 0.00 | 0.00 | 0.00 | 2.79 | 0.00 | 0.00 | 0.00 | 0.00 | 0.00 | 0.00 |
| MARCKS | 0.00 | 0.00 | 0.00 | 0.00 | 0.00 | 0.00 | 0.00 | 0.00 | 0.00 | 0.00 |
| MARCKSL1 | 0.00 | 0.00 | 0.00 | 0.00 | 0.00 | 0.00 | 0.00 | 0.00 | 0.00 | 0.00 |
| MASTL | 0.00 | 0.00 | 0.00 | 0.00 | 0.00 | 0.00 | 0.00 | 0.00 | 0.00 | 0.00 |
| MCL1 | 0.00 | 0.00 | 0.00 | 0.00 | 1.36 | 0.00 | 0.00 | 0.00 | 0.00 | 0.00 |
| MCTP2 | 0.00 | 0.00 | 0.00 | 0.00 | 0.00 | 0.00 | 0.00 | 0.00 | 0.00 | 0.00 |
| MED13 | 0.00 | 2.27 | 0.00 | 0.00 | 0.00 | 0.00 | 0.00 | 0.00 | 0.00 | 0.00 |
| MEF2C | 0.00 | 0.00 | 0.00 | 0.00 | 0.00 | 0.00 | 0.00 | 0.00 | 1.75 | 0.00 |
| METTL14 | 0.00 | 0.00 | 0.00 | 0.00 | 1.58 | 0.00 | 0.00 | 0.00 | 0.00 | 0.00 |
| METTL9 | 0.00 | 0.00 | 0.00 | 0.00 | 0.00 | 0.00 | 0.00 | 0.00 | 0.00 | 0.00 |
| MIF | 0.00 | 0.00 | 0.00 | 0.00 | 0.00 | 0.00 | 0.00 | 0.00 | 0.00 | 0.00 |
| MIRT1 | 0.00 | 0.00 | 0.00 | 0.00 | 1.43 | 0.00 | 0.00 | 0.00 | 0.00 | 0.00 |
| MLANA | 1.73 | 0.00 | 0.00 | 0.00 | 0.00 | 0.00 | 0.00 | 0.00 | 0.00 | 0.00 |
| MLKL | 0.00 | 0.00 | 0.00 | 0.00 | 0.00 | 0.00 | 0.00 | 0.00 | 0.00 | 0.00 |
| MMADHC | 0.00 | 0.00 | 0.00 | 1.37 | 0.00 | 0.00 | 0.00 | 0.00 | 0.00 | 0.00 |
| MMP12 | 0.00 | 0.00 | 1.56 | 0.00 | 0.00 | 0.00 | 0.00 | 0.00 | 0.00 | 0.00 |
| MMP19 | 0.00 | 0.00 | 0.00 | 0.00 | 0.00 | 0.00 | 0.00 | 0.00 | 0.00 | 0.00 |
| MMP8 | 0.00 | 0.00 | 0.00 | 0.00 | 0.00 | 0.00 | 0.00 | 0.00 | 0.00 | 0.00 |
| MMP9 | 0.00 | 0.00 | 0.00 | 0.00 | 0.00 | 0.00 | 0.00 | 0.00 | 0.00 | 0.00 |
| MNDA | 0.00 | 0.00 | 0.00 | 0.00 | 0.00 | 0.00 | 0.00 | 0.00 | 0.00 | 0.00 |
| MNDAL | 0.00 | 0.00 | 0.00 | 0.00 | 0.00 | 0.00 | 0.00 | 0.00 | 0.00 | 0.00 |
| MPEG1 | 2.93 | 0.00 | 0.00 | 0.00 | 0.00 | 0.00 | 0.00 | 0.00 | 1.85 | 0.00 |
| MPP6 | 0.00 | 0.00 | 0.00 | 0.00 | 1.43 | 0.00 | 0.00 | 0.00 | 0.00 | 0.00 |
| MS4A4B | 0.00 | 0.00 | 2.32 | 0.00 | 0.00 | 0.00 | 0.00 | 0.00 | 0.00 | 0.00 |
| MS4A4C | 0.00 | 0.00 | 0.00 | 0.00 | 0.00 | 0.00 | 0.00 | 0.00 | 0.00 | 0.00 |
| MS4A6B | 0.00 | 0.00 | 0.00 | 0.00 | 0.00 | 0.00 | 0.00 | 0.00 | 0.00 | 0.00 |
| MS4A6C | 0.00 | 0.00 | 0.00 | 0.00 | 0.00 | 0.00 | 0.00 | 0.00 | 0.00 | 0.00 |
| MT1 | 0.00 | 0.00 | 0.00 | 0.00 | 0.00 | 5.01 | 0.00 | 0.00 | 0.00 | 0.00 |
| MT-ATP8 | 0.00 | 0.00 | 0.00 | 0.00 | 0.00 | 0.00 | 0.00 | 0.00 | 0.00 | 0.00 |
| MTFP1 | 0.00 | 0.00 | 0.00 | 0.00 | 0.00 | 0.00 | 0.00 | 0.00 | 0.00 | 0.00 |
| MVP | 0.00 | 0.00 | 0.00 | 0.00 | 0.00 | 0.00 | 0.00 | 0.00 | 0.00 | 0.00 |
| MX1 | 0.00 | 0.00 | 0.00 | 0.00 | 0.00 | 0.00 | 0.00 | 0.00 | 0.00 | 0.00 |
| MXD1 | 0.00 | 1.50 | 2.32 | 0.00 | 0.00 | 0.00 | 0.00 | 0.00 | 0.00 | 0.00 |
| MYC | 0.00 | 0.00 | 0.00 | 0.00 | 0.00 | 0.00 | 0.00 | 0.00 | 0.00 | 0.00 |
| MYO1G | 0.00 | 0.00 | 0.00 | 0.00 | 0.00 | 0.00 | 0.00 | 0.00 | 0.00 | 0.00 |
| MZB1 | 0.00 | 0.00 | 2.11 | 0.00 | 0.00 | 0.00 | 0.00 | 0.00 | 0.00 | 1.44 |
| NABP1 | 3.52 | 0.00 | 2.74 | 0.00 | 0.00 | 4.59 | 0.00 | 0.00 | 0.00 | 0.00 |
| NAPSA | 0.00 | 0.00 | 0.00 | 0.00 | 0.00 | 2.30 | 0.00 | 0.00 | 0.00 | 0.00 |
| NCF2 | 0.00 | 0.00 | 0.00 | 0.00 | 0.00 | 0.00 | 1.81 | 0.00 | 0.00 | 0.00 |
| NCOA7 | 0.00 | 0.00 | 0.00 | 0.00 | 0.00 | 0.00 | 0.00 | 0.00 | 0.00 | 0.00 |
| NEAT1 | 3.52 | 2.88 | 0.00 | 0.00 | 0.00 | 0.00 | 0.00 | 0.00 | 0.00 | 0.00 |
| NFKBIA | 0.00 | 1.45 | 0.00 | 3.46 | 0.00 | 0.00 | 0.00 | 0.00 | 0.00 | 0.00 |
| NFKBID | 0.00 | 0.00 | 0.00 | 0.00 | 0.00 | 0.00 | 0.00 | 0.00 | 0.00 | 0.00 |
| NINJ1 | 0.00 | 0.00 | 0.00 | 0.00 | 0.00 | 0.00 | 0.00 | 0.00 | 0.00 | 0.00 |
| NKG7 | 0.00 | 0.00 | 0.00 | 0.00 | 0.00 | 1.52 | 0.00 | 0.00 | 0.00 | 0.00 |
| NLRC5 | 0.00 | 0.00 | 0.00 | 0.00 | 0.00 | 0.00 | 0.00 | 0.00 | 0.00 | 0.00 |
| NMT1 | 0.00 | 0.00 | 0.00 | 2.02 | 0.00 | 0.00 | 0.00 | 0.00 | 0.00 | 0.00 |
| NOCT | 0.00 | 0.00 | 0.00 | 0.00 | 0.00 | 0.00 | 0.00 | 0.00 | 0.00 | 0.00 |
| NPEPPS | 0.00 | 0.00 | 0.00 | 0.00 | 0.00 | 0.00 | 0.00 | 0.00 | 0.00 | 0.00 |
| NT5C3 | 0.00 | 1.35 | 0.00 | 0.00 | 0.00 | 0.00 | 0.00 | 0.00 | 0.00 | 0.00 |
| NUDT4 | 0.00 | 0.00 | 0.00 | 0.00 | 0.00 | 0.00 | 0.00 | 0.00 | 0.00 | 0.00 |
| OAS1A | 0.00 | 0.00 | 0.00 | 0.00 | 0.00 | 0.00 | 0.00 | 0.00 | 0.00 | 0.00 |
| OAS1G | 0.00 | 0.00 | 0.00 | 0.00 | 0.00 | 0.00 | 0.00 | 0.00 | 0.00 | 0.00 |
| OAS3 | 0.00 | 0.00 | 0.00 | 0.00 | 0.00 | 0.00 | 0.00 | 0.00 | 0.00 | 0.00 |
| OASL1 | 0.00 | 0.00 | 0.00 | 0.00 | 0.00 | 0.00 | 0.00 | 0.00 | 0.00 | 0.00 |
| OASL2 | 0.00 | 0.00 | 0.00 | 0.00 | 0.00 | 0.00 | 0.00 | 0.00 | 0.00 | 0.00 |
| OCSTAMP | 0.00 | 0.00 | 0.00 | 0.00 | 0.00 | 0.00 | 0.00 | 0.00 | 0.00 | 0.00 |
| ODC1 | 0.00 | 0.00 | 0.00 | 0.00 | 0.00 | 0.00 | 0.00 | 0.00 | 0.00 | 1.44 |
| OSM | 0.00 | 0.00 | 0.00 | 0.00 | 0.00 | 0.00 | 0.00 | 0.00 | 0.00 | 0.00 |
| P2RY14 | 0.00 | 0.00 | 0.00 | 0.00 | 0.00 | 0.00 | 0.00 | 0.00 | 0.00 | 0.00 |
| P2RY6 | 1.32 | 0.00 | 0.00 | 0.00 | 0.00 | 0.00 | 0.00 | 0.00 | 0.00 | 0.00 |
| PARP12 | 0.00 | 0.00 | 0.00 | 0.00 | 0.00 | 0.00 | 0.00 | 0.00 | 0.00 | 0.00 |
| PARP14 | 0.00 | 1.45 | 0.00 | 0.00 | 0.00 | 0.00 | 0.00 | 0.00 | 0.00 | 0.00 |
| PCMTD1 | 0.00 | 0.00 | 0.00 | 2.05 | 0.00 | 0.00 | 0.00 | 0.00 | 0.00 | 0.00 |
| PCYOX1L | 0.00 | 1.56 | 0.00 | 0.00 | 0.00 | 0.00 | 0.00 | 0.00 | 0.00 | 0.00 |
| PGGT1B | 0.00 | 0.00 | 0.00 | 0.00 | 1.40 | 0.00 | 0.00 | 0.00 | 0.00 | 0.00 |
| PGLYRP1 | 0.00 | 0.00 | 0.00 | 0.00 | 0.00 | 0.00 | 0.00 | 0.00 | 0.00 | 0.00 |
| PHF11B | 0.00 | 0.00 | 1.44 | 0.00 | 0.00 | 0.00 | 0.00 | 0.00 | 0.00 | 0.00 |
| PHF11C | 0.00 | 0.00 | 2.18 | 0.00 | 0.00 | 0.00 | 0.00 | 0.00 | 0.00 | 0.00 |
| PHF11D | 0.00 | 0.00 | 0.00 | 0.00 | 0.00 | 0.00 | 0.00 | 0.00 | 0.00 | 0.00 |
| PHLDA1 | 0.00 | 0.00 | 0.00 | 2.79 | 0.00 | 0.00 | 0.00 | 0.00 | 0.00 | 0.00 |
| PIGL | 0.00 | 0.00 | 0.00 | 0.00 | 0.00 | 0.00 | 0.00 | 0.00 | 0.00 | 0.00 |
| PIK3R6 | 1.62 | 0.00 | 0.00 | 0.00 | 0.00 | 0.00 | 0.00 | 0.00 | 0.00 | 0.00 |
| PILRA | 0.00 | 0.00 | 0.00 | 0.00 | 0.00 | 0.00 | 0.00 | 0.00 | 0.00 | 0.00 |
| PIM1 | 0.00 | 0.00 | 0.00 | 0.00 | 0.00 | 0.00 | 0.00 | 0.00 | 0.00 | 0.00 |
| PJA1 | 0.00 | 0.00 | 0.00 | 2.36 | 0.00 | 0.00 | 0.00 | 0.00 | 0.00 | 0.00 |
| PKIB | 0.00 | 0.00 | 0.00 | 0.00 | 0.00 | 0.00 | 0.00 | 0.00 | 1.60 | 0.00 |
| PLAC8 | 0.00 | 5.66 | 0.00 | 0.00 | 0.00 | 1.50 | 0.00 | 0.00 | 0.00 | 0.00 |
| PLAUR | 0.00 | 0.00 | 0.00 | 0.00 | 0.00 | 0.00 | 0.00 | 0.00 | 0.00 | 0.00 |
| PLBD1 | 2.29 | 0.00 | 0.00 | 0.00 | 0.00 | 0.00 | 0.00 | 0.00 | 0.00 | 0.00 |
| PLIN2 | 0.00 | 0.00 | 0.00 | 0.00 | 0.00 | 0.00 | 0.00 | 0.00 | 0.00 | 0.00 |
| PLK1 | 0.00 | 0.00 | 0.00 | 0.00 | 0.00 | 0.00 | 0.00 | 0.00 | 0.00 | 0.00 |
| PLK2 | 0.00 | 0.00 | 0.00 | 0.00 | 0.00 | 0.00 | 0.00 | 0.00 | 0.00 | 0.00 |
| PLK3 | 0.00 | 0.00 | 0.00 | 0.00 | 0.00 | 0.00 | 0.00 | 0.00 | 0.00 | 0.00 |
| PLSCR1 | 0.00 | 0.00 | 0.00 | 0.00 | 0.00 | 0.00 | 0.00 | 0.00 | 0.00 | 0.00 |
| PLTP | 0.00 | 0.00 | 0.00 | 0.00 | 0.00 | 0.00 | 0.00 | 0.00 | 0.00 | 0.00 |
| PMAIP1 | 0.00 | 0.00 | 0.00 | 0.00 | 0.00 | 0.00 | 0.00 | 0.00 | 0.00 | 0.00 |
| PMEL | 1.52 | 0.00 | 0.00 | 0.00 | 0.00 | 1.62 | 0.00 | 0.00 | 0.00 | 0.00 |
| PML | 0.00 | 0.00 | 0.00 | 0.00 | 0.00 | 0.00 | 0.00 | 0.00 | 0.00 | 0.00 |
| PNP | 0.00 | 0.00 | 0.00 | 0.00 | 0.00 | 0.00 | 0.00 | 0.00 | 0.00 | 0.00 |
| PNRC1 | 0.00 | 0.00 | 0.00 | 0.00 | 0.00 | 0.00 | 0.00 | 0.00 | 0.00 | 0.00 |
| POLE4 | 0.00 | 0.00 | 0.00 | 0.00 | 0.00 | 0.00 | 0.00 | 0.00 | 0.00 | 0.00 |
| PPP1R10 | 0.00 | 0.00 | 0.00 | 0.00 | 0.00 | 0.00 | 0.00 | 0.00 | 0.00 | 0.00 |
| PPP1R15A | 5.22 | 5.58 | 0.00 | 7.18 | 1.80 | 0.00 | 0.00 | 0.00 | 0.00 | 0.00 |
| PPP1R16B | 0.00 | 0.00 | 0.00 | 0.00 | 0.00 | 0.00 | 0.00 | 0.00 | 0.00 | 0.00 |
| PPP2R3C | 0.00 | 0.00 | 0.00 | 0.00 | 0.00 | 0.00 | 0.00 | 0.00 | 0.00 | 0.00 |
| PPP6R3 | 0.00 | 0.00 | 0.00 | 0.00 | 0.00 | 0.00 | 0.00 | 0.00 | 0.00 | 0.00 |
| PRDM2 | 0.00 | 0.00 | 0.00 | 0.00 | 0.00 | 0.00 | 0.00 | 0.00 | 0.00 | 0.00 |
| PRDX6 | 0.00 | 0.00 | 0.00 | 0.00 | 0.00 | 0.00 | 0.00 | 0.00 | 0.00 | 0.00 |
| PRKCA | 0.00 | 0.00 | 0.00 | 0.00 | 0.00 | 0.00 | 0.00 | 0.00 | 0.00 | 0.00 |
| PSME2 | 0.00 | 0.00 | 1.40 | 0.00 | 0.00 | 0.00 | 0.00 | 0.00 | 0.00 | 0.00 |
| PTAFR | 0.00 | 0.00 | 0.00 | 0.00 | 0.00 | 0.00 | 0.00 | 0.00 | 0.00 | 0.00 |
| PTGDS | 0.00 | 0.00 | 1.34 | 0.00 | 0.00 | 0.00 | 0.00 | 0.00 | 0.00 | 0.00 |
| PTGS2 | 0.00 | 0.00 | 0.00 | 0.00 | 0.00 | 0.00 | 0.00 | 0.00 | 0.00 | 0.00 |
| PTP4A1 | 0.00 | 0.00 | 0.00 | 0.00 | 0.00 | 0.00 | 0.00 | 0.00 | 0.00 | 0.00 |
| PTPN22 | 0.00 | 0.00 | 0.00 | 0.00 | 0.00 | 0.00 | 0.00 | 0.00 | 0.00 | 0.00 |
| PVT1 | 0.00 | 0.00 | 0.00 | 0.00 | 0.00 | 0.00 | 0.00 | 0.00 | 0.00 | 1.32 |
| PYDC3 | 0.00 | 0.00 | 0.00 | 0.00 | 0.00 | 0.00 | 0.00 | 0.00 | 0.00 | 0.00 |
| PYDC4 | 0.00 | 0.00 | 2.00 | 0.00 | 0.00 | 1.48 | 0.00 | 0.00 | 0.00 | 0.00 |
| PYHIN1 | 0.00 | 0.00 | 0.00 | 0.00 | 0.00 | 0.00 | 0.00 | 0.00 | 0.00 | 1.35 |
| RAB10OS | 1.37 | 0.00 | 0.00 | 0.00 | 0.00 | 0.00 | 0.00 | 0.00 | 0.00 | 0.00 |
| RAB28 | 0.00 | 0.00 | 0.00 | 0.00 | 1.30 | 0.00 | 0.00 | 0.00 | 0.00 | 0.00 |
| RAB3IL1 | 2.57 | 0.00 | 0.00 | 0.00 | 0.00 | 0.00 | 0.00 | 0.00 | 0.00 | 0.00 |
| RAMP1 | 0.00 | 0.00 | 1.33 | 0.00 | 0.00 | 0.00 | 0.00 | 0.00 | 0.00 | 0.00 |
| RBM39 | 1.92 | 1.35 | 0.00 | 1.63 | 0.00 | 0.00 | 0.00 | 0.00 | 0.00 | 0.00 |
| RBPJ | 0.00 | 0.00 | 0.00 | 0.00 | 0.00 | 0.00 | 0.00 | 0.00 | 0.00 | 0.00 |
| RETNLG | 0.00 | 0.00 | 0.00 | 1.51 | 0.00 | 0.00 | 0.00 | 0.00 | 0.00 | 0.00 |
| RGCC | 0.00 | 0.00 | 0.00 | 0.00 | 0.00 | 0.00 | 0.00 | 0.00 | 0.00 | 0.00 |
| RGL1 | 0.00 | 0.00 | 0.00 | 0.00 | 0.00 | 0.00 | 0.00 | 0.00 | 0.00 | 0.00 |
| RGS1 | 0.00 | 0.00 | 1.42 | 3.41 | 0.00 | 0.00 | 0.00 | 0.00 | 0.00 | 1.41 |
| RGS16 | 0.00 | 2.10 | 0.00 | 0.00 | 0.00 | 0.00 | 0.00 | 0.00 | 0.00 | 0.00 |
| RHOV | 0.00 | 0.00 | 0.00 | 0.00 | 0.00 | 0.00 | 0.00 | 0.00 | 0.00 | 0.00 |
| RIF1 | 0.00 | 0.00 | 0.00 | 0.00 | 0.00 | 0.00 | 0.00 | 0.00 | 0.00 | 0.00 |
| RIN2 | 0.00 | 0.00 | 0.00 | 0.00 | 0.00 | 0.00 | 0.00 | 0.00 | 0.00 | 0.00 |
| RINL | 1.44 | 0.00 | 0.00 | 0.00 | 0.00 | 0.00 | 0.00 | 0.00 | 0.00 | 0.00 |
| RNASE6 | 0.00 | 0.00 | 0.00 | 0.00 | 0.00 | 0.00 | 0.00 | 0.00 | 3.08 | 0.00 |
| RNF138 | 3.26 | 0.00 | 0.00 | 0.00 | 0.00 | 0.00 | 0.00 | 0.00 | 0.00 | 0.00 |
| RNF213 | 0.00 | 0.00 | 0.00 | 0.00 | 0.00 | 0.00 | 0.00 | 0.00 | 0.00 | 0.00 |
| RNF34 | 0.00 | 0.00 | 0.00 | 0.00 | 0.00 | 0.00 | 0.00 | 0.00 | 0.00 | 0.00 |
| ROCK2 | 0.00 | 0.00 | 0.00 | 0.00 | 2.24 | 0.00 | 0.00 | 0.00 | 0.00 | 0.00 |
| RP23-6I17.1 | 0.00 | 0.00 | 0.00 | 0.00 | 0.00 | 0.00 | 0.00 | 0.00 | 0.00 | 0.00 |
| RPIA | 0.00 | 0.00 | 0.00 | 0.00 | 0.00 | 0.00 | 0.00 | 0.00 | 0.00 | 0.00 |
| RPL35 | 2.32 | 1.60 | 0.00 | 0.00 | 0.00 | 0.00 | 0.00 | 0.00 | 0.00 | 0.00 |
| RPL36-PS3 | 0.00 | 0.00 | 0.00 | 1.58 | 0.00 | 0.00 | 0.00 | 0.00 | 0.00 | 0.00 |
| RTP4 | 2.77 | 3.03 | 2.68 | 1.68 | 0.00 | 0.00 | 0.00 | 0.00 | 0.00 | 0.00 |
| RUFY3 | 0.00 | 0.00 | 0.00 | 0.00 | 0.00 | 0.00 | 0.00 | 0.00 | 0.00 | 0.00 |
| S100A11 | 0.00 | 0.00 | 0.00 | 0.00 | 0.00 | 0.00 | 0.00 | 0.00 | 0.00 | 0.00 |
| S100A6 | 1.97 | 0.00 | 0.00 | 0.00 | 0.00 | 0.00 | 0.00 | 0.00 | 0.00 | 0.00 |
| S100A9 | 0.00 | 0.00 | 2.52 | 3.20 | 1.85 | 2.30 | 0.00 | 0.00 | 0.00 | 0.00 |
| SACS | 0.00 | 0.00 | 0.00 | 0.00 | 2.72 | 0.00 | 0.00 | 0.00 | 0.00 | 0.00 |
| SAG | 0.00 | 0.00 | 0.00 | 0.00 | 0.00 | 0.00 | 0.00 | 0.00 | 0.00 | 0.00 |
| SAMHD1 | 1.49 | 0.00 | 2.77 | 0.00 | 0.00 | 1.31 | 0.00 | 0.00 | 0.00 | 0.00 |
| SAP30L | 0.00 | 0.00 | 0.00 | 0.00 | 1.46 | 0.00 | 0.00 | 0.00 | 0.00 | 0.00 |
| SARAF | 0.00 | 0.00 | 0.00 | 0.00 | 0.00 | 0.00 | 0.00 | 0.00 | 0.00 | 0.00 |
| SATB1 | 0.00 | 0.00 | 0.00 | 0.00 | 0.00 | 0.00 | 0.00 | 0.00 | 0.00 | 0.00 |
| SDC3 | 0.00 | 0.00 | 0.00 | 0.00 | 0.00 | 0.00 | 0.00 | 0.00 | 0.00 | 0.00 |
| SDC4 | 0.00 | 0.00 | 0.00 | 0.00 | 0.00 | 3.74 | 0.00 | 0.00 | 0.00 | 0.00 |
| SDHAF1 | 0.00 | 0.00 | 0.00 | 0.00 | 0.00 | 0.00 | 0.00 | 0.00 | 0.00 | 0.00 |
| SEC22A | 0.00 | 0.00 | 0.00 | 0.00 | 0.00 | 0.00 | 0.00 | 0.00 | 0.00 | 0.00 |
| SELENBP1 | 0.00 | 0.00 | 0.00 | 0.00 | 0.00 | 1.58 | 0.00 | 0.00 | 0.00 | 0.00 |
| SENP6 | 0.00 | 0.00 | 0.00 | 0.00 | 2.24 | 0.00 | 0.00 | 0.00 | 0.00 | 0.00 |
| SEPT11 | 0.00 | 0.00 | 1.31 | 0.00 | 0.00 | 0.00 | 0.00 | 0.00 | 0.00 | 0.00 |
| SERINC3 | 0.00 | 0.00 | 0.00 | 0.00 | 0.00 | 0.00 | 0.00 | 0.00 | 0.00 | 0.00 |
| SERTAD1 | 2.44 | 0.00 | 0.00 | 0.00 | 0.00 | 0.00 | 0.00 | 0.00 | 0.00 | 0.00 |
| SETX | 1.94 | 0.00 | 0.00 | 0.00 | 0.00 | 0.00 | 0.00 | 0.00 | 0.00 | 0.00 |
| SGK1 | 0.00 | 0.00 | 0.00 | 1.68 | 0.00 | 0.00 | 0.00 | 0.00 | 0.00 | 0.00 |
| SIAH2 | 0.00 | 0.00 | 0.00 | 0.00 | 0.00 | 0.00 | 0.00 | 0.00 | 0.00 | 0.00 |
| SIGLECH | 0.00 | 0.00 | 0.00 | 0.00 | 0.00 | 0.00 | 0.00 | 0.00 | 0.00 | 0.00 |
| SIRPB1C | 0.00 | 0.00 | 0.00 | 0.00 | 0.00 | 0.00 | 0.00 | 0.00 | 0.00 | 1.40 |
| SLAMF9 | 0.00 | 0.00 | 0.00 | 0.00 | 0.00 | 0.00 | 0.00 | 0.00 | 0.00 | 0.00 |
| SLC7A11 | 0.00 | 0.00 | 0.00 | 0.00 | 0.00 | 0.00 | 0.00 | 0.00 | 0.00 | 0.00 |
| SLFN1 | 0.00 | 0.00 | 2.77 | 0.00 | 0.00 | 0.00 | 0.00 | 0.00 | 0.00 | 0.00 |
| SLFN2 | 0.00 | 0.00 | 0.00 | 0.00 | 0.00 | 0.00 | 0.00 | 0.00 | 0.00 | 0.00 |
| SLFN5 | 2.42 | 0.00 | 0.00 | 0.00 | 0.00 | 0.00 | 0.00 | 0.00 | 0.00 | 0.00 |
| SLFN8 | 0.00 | 1.35 | 0.00 | 0.00 | 0.00 | 0.00 | 0.00 | 0.00 | 0.00 | 0.00 |
| SLPI | 0.00 | 0.00 | 0.00 | 0.00 | 0.00 | 0.00 | 0.00 | 0.00 | 0.00 | 0.00 |
| SMIM14 | 0.00 | 0.00 | 0.00 | 0.00 | 0.00 | 0.00 | 0.00 | 0.00 | 2.01 | 0.00 |
| SMIM5 | 0.00 | 0.00 | 0.00 | 0.00 | 0.00 | 0.00 | 0.00 | 0.00 | 1.60 | 0.00 |
| SMOX | 0.00 | 0.00 | 0.00 | 0.00 | 0.00 | 0.00 | 0.00 | 0.00 | 0.00 | 0.00 |
| SNX9 | 0.00 | 0.00 | 0.00 | 0.00 | 0.00 | 0.00 | 0.00 | 0.00 | 0.00 | 0.00 |
| SOCS1 | 0.00 | 0.00 | 0.00 | 0.00 | 0.00 | 0.00 | 0.00 | 0.00 | 0.00 | 0.00 |
| SOCS3 | 3.46 | 0.00 | 0.00 | 5.44 | 0.00 | 0.00 | 0.00 | 0.00 | 0.00 | 0.00 |
| SP110 | 0.00 | 0.00 | 0.00 | 0.00 | 0.00 | 0.00 | 0.00 | 0.00 | 0.00 | 0.00 |
| SP140 | 0.00 | 0.00 | 0.00 | 0.00 | 0.00 | 0.00 | 0.00 | 0.00 | 0.00 | 0.00 |
| SPRED1 | 0.00 | 0.00 | 0.00 | 0.00 | 0.00 | 0.00 | 0.00 | 0.00 | 0.00 | 0.00 |
| SPRY2 | 0.00 | 2.11 | 0.00 | 0.00 | 0.00 | 0.00 | 0.00 | 0.00 | 0.00 | 0.00 |
| SPTY2D1 | 0.00 | 0.00 | 0.00 | 0.00 | 0.00 | 0.00 | 0.00 | 0.00 | 0.00 | 0.00 |
| SQLE | 0.00 | 0.00 | 0.00 | 0.00 | 0.00 | 0.00 | 0.00 | 0.00 | 0.00 | 0.00 |
| SRGN | 0.00 | 0.00 | 0.00 | 0.00 | 0.00 | 0.00 | 0.00 | 0.00 | 0.00 | 0.00 |
| SRSF5 | 0.00 | 0.00 | 0.00 | 0.00 | 0.00 | 0.00 | 0.00 | 0.00 | 0.00 | 0.00 |
| SSH2 | 0.00 | 0.00 | 0.00 | 2.51 | 0.00 | 1.38 | 0.00 | 0.00 | 0.00 | 0.00 |
| ST13 | 0.00 | 0.00 | 0.00 | 0.00 | 0.00 | 0.00 | 0.00 | 0.00 | 0.00 | 0.00 |
| ST3GAL6 | 0.00 | 0.00 | 0.00 | 0.00 | 0.00 | 0.00 | 0.00 | 0.00 | 0.00 | 1.35 |
| STAT1 | 0.00 | 1.71 | 0.00 | 0.00 | 0.00 | 2.35 | 0.00 | 0.00 | 0.00 | 0.00 |
| STAT2 | 0.00 | 0.00 | 0.00 | 0.00 | 0.00 | 0.00 | 0.00 | 0.00 | 0.00 | 0.00 |
| SV2C | 0.00 | 0.00 | 0.00 | 0.00 | 0.00 | 1.54 | 0.00 | 0.00 | 0.00 | 0.00 |
| SYK | 0.00 | 0.00 | 0.00 | 0.00 | 0.00 | 1.75 | 0.00 | 0.00 | 0.00 | 0.00 |
| SYNE1 | 0.00 | 0.00 | 0.00 | 0.00 | 0.00 | 0.00 | 0.00 | 0.00 | 0.00 | 0.00 |
| SYNGR1 | 0.00 | 0.00 | 0.00 | 0.00 | 0.00 | 1.67 | 0.00 | 0.00 | 0.00 | 0.00 |
| SYNJ1 | 0.00 | 0.00 | 0.00 | 0.00 | 0.00 | 0.00 | 0.00 | 0.00 | 0.00 | 0.00 |
| TAP1 | 0.00 | 0.00 | 0.00 | 0.00 | 0.00 | 0.00 | 0.00 | 0.00 | 0.00 | 0.00 |
| TAPBP | 0.00 | 0.00 | 0.00 | 0.00 | 0.00 | 0.00 | 0.00 | 0.00 | 0.00 | 0.00 |
| TBC1D8 | 0.00 | 0.00 | 0.00 | 0.00 | 0.00 | 0.00 | 0.00 | 0.00 | 2.83 | 0.00 |
| TCF4 | 0.00 | 0.00 | 0.00 | 0.00 | 0.00 | 0.00 | 0.00 | 0.00 | 2.46 | 0.00 |
| TESC | 0.00 | 0.00 | 0.00 | 0.00 | 0.00 | 0.00 | 0.00 | 0.00 | 0.00 | 1.44 |
| TGTP2 | 0.00 | 0.00 | 1.78 | 0.00 | 0.00 | 0.00 | 0.00 | 0.00 | 0.00 | 0.00 |
| THBS1 | 0.00 | 0.00 | 0.00 | 0.00 | 0.00 | 0.00 | 0.00 | 0.00 | 0.00 | 0.00 |
| THEMIS2 | 0.00 | 0.00 | 0.00 | 0.00 | 0.00 | 0.00 | 0.00 | 0.00 | 0.00 | 0.00 |
| TIFA | 0.00 | 0.00 | 0.00 | 0.00 | 0.00 | 0.00 | 0.00 | 0.00 | 1.84 | 0.00 |
| TIPARP | 0.00 | 0.00 | 0.00 | 0.00 | 0.00 | 0.00 | 0.00 | 0.00 | 0.00 | 0.00 |
| TLN1 | 1.60 | 0.00 | 0.00 | 0.00 | 0.00 | 0.00 | 0.00 | 0.00 | 0.00 | 0.00 |
| TMEM106A | 2.44 | 0.00 | 0.00 | 0.00 | 0.00 | 0.00 | 0.00 | 0.00 | 0.00 | 0.00 |
| TMEM106B | 0.00 | 1.35 | 0.00 | 0.00 | 0.00 | 0.00 | 0.00 | 0.00 | 0.00 | 0.00 |
| TMEM158 | 0.00 | 0.00 | 0.00 | 0.00 | 0.00 | 0.00 | 0.00 | 0.00 | 0.00 | 0.00 |
| TMEM176B | 0.00 | 0.00 | 0.00 | 0.00 | 0.00 | 0.00 | 0.00 | 0.00 | 0.00 | 0.00 |
| TMEM229B | 1.37 | 0.00 | 0.00 | 0.00 | 0.00 | 0.00 | 0.00 | 0.00 | 0.00 | 0.00 |
| TMEM55B | 0.00 | 0.00 | 0.00 | 1.47 | 0.00 | 0.00 | 0.00 | 0.00 | 0.00 | 0.00 |
| TNFAIP3 | 2.39 | 0.00 | 0.00 | 0.00 | 0.00 | 0.00 | 0.00 | 0.00 | 0.00 | 0.00 |
| TNFRSF13B | 0.00 | 0.00 | 0.00 | 0.00 | 0.00 | 0.00 | 0.00 | 0.00 | 0.00 | 0.00 |
| TNFRSF18 | 0.00 | 0.00 | 0.00 | 0.00 | 1.61 | 0.00 | 0.00 | 0.00 | 0.00 | 0.00 |
| TNFRSF25 | 0.00 | 0.00 | 1.31 | 0.00 | 0.00 | 0.00 | 0.00 | 0.00 | 0.00 | 0.00 |
| TNFRSF4 | 0.00 | 0.00 | 0.00 | 0.00 | 0.00 | 0.00 | 0.00 | 0.00 | 0.00 | 3.15 |
| TNFRSF9 | 0.00 | 0.00 | 0.00 | 0.00 | 0.00 | 0.00 | 0.00 | 0.00 | 0.00 | 0.00 |
| TNFSF10 | 0.00 | 0.00 | 0.00 | 0.00 | 0.00 | 0.00 | 0.00 | 0.00 | 0.00 | 0.00 |
| TNKS2 | 0.00 | 0.00 | 0.00 | 0.00 | 1.58 | 0.00 | 0.00 | 0.00 | 0.00 | 0.00 |
| TOB1 | 3.44 | 0.00 | 0.00 | 0.00 | 0.00 | 0.00 | 0.00 | 0.00 | 0.00 | 0.00 |
| TOR1AIP1 | 0.00 | 0.00 | 0.00 | 0.00 | 0.00 | 0.00 | 0.00 | 0.00 | 0.00 | 0.00 |
| TPI1 | 0.00 | 0.00 | 0.00 | 0.00 | 0.00 | 0.00 | 0.00 | 0.00 | 0.00 | 0.00 |
| TPM4 | 0.00 | 0.00 | 1.51 | 0.00 | 0.00 | 0.00 | 0.00 | 0.00 | 0.00 | 0.00 |
| TRA2B | 4.30 | 3.04 | 2.36 | 4.62 | 0.00 | 0.00 | 0.00 | 0.00 | 0.00 | 0.00 |
| TRAFD1 | 0.00 | 0.00 | 0.00 | 0.00 | 0.00 | 0.00 | 0.00 | 0.00 | 0.00 | 0.00 |
| TRAPPC10 | 0.00 | 0.00 | 0.00 | 0.00 | 1.60 | 0.00 | 0.00 | 0.00 | 0.00 | 0.00 |
| TREM1 | 0.00 | 0.00 | 0.00 | 0.00 | 0.00 | 0.00 | 0.00 | 0.00 | 0.00 | 0.00 |
| TRIB1 | 0.00 | 0.00 | 0.00 | 0.00 | 0.00 | 0.00 | 0.00 | 0.00 | 0.00 | 0.00 |
| TRIM14 | 0.00 | 0.00 | 0.00 | 0.00 | 0.00 | 0.00 | 0.00 | 0.00 | 0.00 | 0.00 |
| TRIM30A | 0.00 | 0.00 | 0.00 | 0.00 | 0.00 | 0.00 | 0.00 | 0.00 | 0.00 | 0.00 |
| TRIM30B | 0.00 | 0.00 | 0.00 | 0.00 | 0.00 | 0.00 | 0.00 | 0.00 | 0.00 | 0.00 |
| TRIM5 | 0.00 | 0.00 | 0.00 | 0.00 | 0.00 | 0.00 | 0.00 | 0.00 | 0.00 | 0.00 |
| TRIM56 | 1.64 | 0.00 | 0.00 | 0.00 | 0.00 | 0.00 | 0.00 | 0.00 | 0.00 | 0.00 |
| TRIP12 | 1.68 | 0.00 | 0.00 | 0.00 | 0.00 | 0.00 | 0.00 | 0.00 | 0.00 | 0.00 |
| TSC22D3 | 2.21 | 0.00 | 0.00 | 0.00 | 0.00 | 0.00 | 0.00 | 0.00 | 0.00 | 0.00 |
| TSIX | 0.00 | 0.00 | 0.00 | 1.71 | 0.00 | 0.00 | 0.00 | 0.00 | 0.00 | 0.00 |
| TTC37 | 0.00 | 0.00 | 0.00 | 0.00 | 3.12 | 0.00 | 0.00 | 0.00 | 0.00 | 0.00 |
| TUBA4A | 0.00 | 0.00 | 0.00 | 0.00 | 0.00 | 0.00 | 0.00 | 0.00 | 0.00 | 0.00 |
| TXK | 0.00 | 0.00 | 0.00 | 0.00 | 0.00 | 0.00 | 0.00 | 0.00 | 0.00 | 0.00 |
| TYRP1 | 0.00 | 0.00 | 0.00 | 0.00 | 0.00 | 0.00 | 0.00 | 0.00 | 0.00 | 0.00 |
| UBASH3B | 0.00 | 0.00 | 0.00 | 0.00 | 0.00 | 0.00 | 0.00 | 0.00 | 0.00 | 0.00 |
| UBB | 0.00 | 0.00 | 0.00 | 0.00 | 1.43 | 0.00 | 0.00 | 0.00 | 0.00 | 0.00 |
| UBC | 1.77 | 0.00 | 0.00 | 1.96 | 0.00 | 0.00 | 0.00 | 0.00 | 0.00 | 0.00 |
| UBE2L6 | 0.00 | 0.00 | 0.00 | 0.00 | 0.00 | 0.00 | 0.00 | 0.00 | 0.00 | 0.00 |
| UBE2S | 0.00 | 0.00 | 0.00 | 1.39 | 0.00 | 0.00 | 0.00 | 0.00 | 0.00 | 0.00 |
| UCK2 | 0.00 | 0.00 | 0.00 | 0.00 | 0.00 | 0.00 | 0.00 | 0.00 | 0.00 | 0.00 |
| UPP1 | 0.00 | 0.00 | 0.00 | 0.00 | 0.00 | 0.00 | 0.00 | 0.00 | 0.00 | 0.00 |
| USP12 | 0.00 | 0.00 | 0.00 | 0.00 | 0.00 | 0.00 | 0.00 | 0.00 | 0.00 | 0.00 |
| USP18 | 0.00 | 0.00 | 0.00 | 1.36 | 0.00 | 0.00 | 0.00 | 0.00 | 0.00 | 0.00 |
| VAV3 | 0.00 | 0.00 | 0.00 | 0.00 | 0.00 | 0.00 | 0.00 | 0.00 | 0.00 | 0.00 |
| VEGFA | 0.00 | 0.00 | 0.00 | 0.00 | 0.00 | 0.00 | 0.00 | 0.00 | 0.00 | 0.00 |
| VPS37B | 0.00 | 0.00 | 0.00 | 0.00 | 0.00 | 0.00 | 0.00 | 0.00 | 0.00 | 0.00 |
| WDR91 | 0.00 | 0.00 | 0.00 | 0.00 | 0.00 | 0.00 | 0.00 | 0.00 | 0.00 | 0.00 |
| WFDC17 | 0.00 | 0.00 | 0.00 | 0.00 | 0.00 | 0.00 | 0.00 | 0.00 | 0.00 | 0.00 |
| XAF1 | 0.00 | 0.00 | 2.29 | 0.00 | 0.00 | 0.00 | 0.00 | 0.00 | 0.00 | 0.00 |
| XPO6 | 0.00 | 0.00 | 0.00 | 0.00 | 0.00 | 0.00 | 0.00 | 0.00 | 0.00 | 0.00 |
| XPR1 | 0.00 | 0.00 | 0.00 | 0.00 | 0.00 | 0.00 | 0.00 | 0.00 | 0.00 | 0.00 |
| ZBP1 | 1.52 | 0.00 | 2.02 | 0.00 | 0.00 | 0.00 | 0.00 | 0.00 | 0.00 | 0.00 |
| ZFHX2 | 0.00 | 0.00 | 0.00 | 0.00 | 0.00 | 0.00 | 0.00 | 0.00 | 0.00 | 0.00 |
| ZFP106 | 0.00 | 0.00 | 0.00 | 0.00 | 0.00 | 0.00 | 0.00 | 0.00 | 0.00 | 0.00 |
| ZFP275 | 0.00 | 0.00 | 1.42 | 0.00 | 0.00 | 0.00 | 0.00 | 0.00 | 0.00 | 0.00 |
| ZFP36L2 | 0.00 | 0.00 | 0.00 | 0.00 | 0.00 | 0.00 | 0.00 | 0.00 | 0.00 | 0.00 |
| ZFP646 | 0.00 | 0.00 | 0.00 | 0.00 | 1.99 | 0.00 | 0.00 | 0.00 | 0.00 | 0.00 |
| ZFP931 | 0.00 | 0.00 | 1.42 | 0.00 | 0.00 | 0.00 | 0.00 | 0.00 | 0.00 | 0.00 |
| ZFP954 | 0.00 | 0.00 | 0.00 | 0.00 | 1.78 | 0.00 | 0.00 | 0.00 | 0.00 | 0.00 |
| ZFX | 0.00 | 0.00 | 0.00 | 0.00 | 0.00 | 0.00 | 0.00 | 0.00 | 0.00 | 0.00 |
| ZMYND15 | 0.00 | 0.00 | 0.00 | 0.00 | 0.00 | 0.00 | 0.00 | 0.00 | 0.00 | 0.00 |
| ZNFX1 | 0.00 | 0.00 | 0.00 | 0.00 | 0.00 | 0.00 | 0.00 | 0.00 | 0.00 | 0.00 |
| ZWINT | 0.00 | 0.00 | 0.00 | 0.00 | 0.00 | 0.00 | 0.00 | 0.00 | 0.00 | 0.00 |

Table 3A-3B. Differentially Expressed Genes Associated with Tumor Size

TABLE 3A
Non-T cell clusters

| | Non- | Non- | Non- | Non- | Non- | Non- | Non- | Non- | Non- | Non- | Non- | Non- |
|---|---|---|---|---|---|---|---|---|---|---|---|---|
| Gene | T_0 | T_1 | T_10 | T_11 | T_2 | T_3 | T_4 | T_5 | T_6 | T_7 | T_8 | T_9 |
| MALT1 | 0.00 | 0.00 | 0.00 | 0.00 | 0.00 | 0.00 | 0.00 | 0.00 | 0.00 | 0.00 | 0.00 | 0.00 |
| ITGA1 | 0.00 | 0.00 | 0.00 | 0.00 | 0.00 | 0.00 | 0.00 | 0.00 | 0.00 | 0.00 | 0.00 | 0.00 |
| ETNK1 | 0.00 | 5.01 | 0.00 | 0.00 | 0.00 | 0.00 | 0.00 | 0.00 | 0.00 | 0.00 | 0.00 | 0.00 |
| HBB-BS | 6.43 | 3.09 | 5.45 | 0.00 | 0.00 | 5.38 | 3.65 | 4.96 | 0.00 | 0.00 | 0.00 | 6.08 |
| SP4 | 0.00 | 0.00 | 0.00 | 0.00 | 0.00 | 0.00 | 0.00 | 0.00 | 0.00 | 0.00 | 0.00 | 0.00 |
| ATG7 | 0.00 | 0.00 | 0.00 | 0.00 | 0.00 | 0.00 | 0.00 | 0.00 | 0.00 | 0.00 | 0.00 | 0.00 |
| PYDC4 | 0.00 | 7.08 | 0.00 | 0.00 | 0.00 | 0.00 | 6.06 | 4.55 | 0.00 | 0.00 | 0.00 | 0.00 |
| PYDC3 | 0.00 | 6.08 | 0.00 | 0.00 | 0.00 | 0.00 | 0.00 | 0.00 | 0.00 | 0.00 | 4.33 | 0.00 |
| PENK | 0.00 | 0.00 | 0.00 | 0.00 | 0.00 | 0.00 | 0.00 | 0.00 | 0.00 | 0.00 | 0.00 | 0.00 |
| IFITM3 | 3.86 | 4.56 | 0.00 | 0.00 | 0.00 | 0.00 | 11.12 | 0.00 | 5.61 | 7.11 | 0.00 | 0.00 |
| SAMD9L | 0.00 | 4.71 | 0.00 | 0.00 | 0.00 | 4.09 | 6.92 | 0.00 | 3.22 | 0.00 | 4.08 | 0.00 |
| CCL8 | 0.00 | 0.00 | 0.00 | 0.00 | 0.00 | 5.66 | 0.00 | 0.00 | 0.00 | 0.00 | 0.00 | 0.00 |
| ZCCHC6 | 0.00 | 0.00 | 0.00 | 0.00 | 0.00 | 0.00 | 0.00 | 0.00 | 0.00 | 0.00 | 0.00 | 0.00 |
| C1QB | 0.00 | 6.54 | 0.00 | 0.00 | 0.00 | 4.01 | 0.00 | 0.00 | 7.88 | 0.00 | 0.00 | 0.00 |
| MIR142HG | 0.00 | 0.00 | 0.00 | 0.00 | 0.00 | 0.00 | 0.00 | 0.00 | 0.00 | 0.00 | 0.00 | 0.00 |
| PYHIN1 | 0.00 | 0.00 | 0.00 | 0.00 | 0.00 | 0.00 | 4.76 | 0.00 | 0.00 | 0.00 | 0.00 | 0.00 |
| SEPT6 | 0.00 | 0.00 | 0.00 | 0.00 | 0.00 | 0.00 | 0.00 | 0.00 | 0.00 | 0.00 | 0.00 | 0.00 |
| CCL6 | 0.00 | 3.42 | 0.00 | 0.00 | 0.00 | 0.00 | 0.00 | 0.00 | 0.00 | 3.74 | 0.00 | 0.00 |
| XAF1 | 0.00 | 0.00 | 0.00 | 0.00 | 0.00 | 0.00 | 4.65 | 0.00 | 3.47 | 0.00 | 0.00 | 0.00 |
| LGALS7 | 0.00 | 0.00 | 0.00 | 0.00 | 0.00 | 0.00 | 0.00 | 0.00 | 0.00 | 0.00 | 0.00 | 0.00 |
| DCT | 0.00 | 0.00 | 0.00 | 0.00 | 0.00 | 0.00 | 0.00 | 0.00 | 0.00 | 0.00 | 0.00 | 0.00 |
| ZBP1 | 0.00 | 3.40 | 4.87 | 0.00 | 0.00 | 0.00 | 8.60 | 3.60 | 6.51 | 0.00 | 0.00 | 0.00 |
| ASXL2 | 0.00 | 0.00 | 0.00 | 0.00 | 0.00 | 0.00 | 0.00 | 0.00 | 0.00 | 0.00 | 0.00 | 0.00 |
| CHD2 | 0.00 | 0.00 | 0.00 | 0.00 | 0.00 | 0.00 | 0.00 | 0.00 | 0.00 | 0.00 | 0.00 | 0.00 |
| S100A8 | 26.23 | 4.96 | 0.00 | 0.00 | 0.00 | 35.99 | 6.75 | 25.87 | 5.17 | 9.02 | 16.72 | 127.78 |
| XRN1 | 0.00 | 0.00 | 0.00 | 0.00 | 0.00 | 0.00 | 0.00 | 0.00 | 0.00 | 0.00 | 0.00 | 0.00 |
| PMS1 | 0.00 | 0.00 | 0.00 | 0.00 | 0.00 | 0.00 | 0.00 | 0.00 | 0.00 | 0.00 | 0.00 | 0.00 |
| GM42418 | 0.00 | 3.55 | 0.00 | 0.00 | 4.37 | 0.00 | 3.10 | 0.00 | 4.60 | 0.00 | 0.00 | 3.69 |
| BTAF1 | 0.00 | 0.00 | 0.00 | 0.00 | 0.00 | 0.00 | 0.00 | 0.00 | 0.00 | 0.00 | 0.00 | 0.00 |
| GM26532 | 0.00 | 4.55 | 0.00 | 0.00 | 0.00 | 0.00 | 0.00 | 5.95 | 4.49 | 0.00 | 0.00 | 0.00 |
| AIF1 | 0.00 | 6.34 | 0.00 | 0.00 | 0.00 | 0.00 | 0.00 | 0.00 | 8.25 | 0.00 | 4.08 | 0.00 |
| VPS13C | 0.00 | 3.32 | 0.00 | 0.00 | 0.00 | 0.00 | 0.00 | 0.00 | 0.00 | 0.00 | 0.00 | 0.00 |
| TFF1 | 0.00 | 0.00 | 0.00 | 0.00 | 0.00 | 0.00 | 4.04 | 0.00 | 6.88 | 0.00 | 0.00 | 0.00 |
| STAT1 | 0.00 | 4.08 | 4.18 | 0.00 | 0.00 | 0.00 | 4.31 | 0.00 | 3.88 | 0.00 | 4.47 | 5.01 |
| AHNAK | 0.00 | 3.10 | 5.47 | 0.00 | 0.00 | 4.64 | 0.00 | 0.00 | 0.00 | 4.57 | 0.00 | 0.00 |
| H2-EB1 | 0.00 | 6.29 | 0.00 | 0.00 | 0.00 | 0.00 | 0.00 | 0.00 | 6.75 | 0.00 | 6.63 | 8.47 |
| CHKA | 0.00 | 0.00 | 0.00 | 0.00 | 0.00 | 0.00 | 0.00 | 0.00 | 0.00 | 0.00 | 0.00 | 0.00 |
| IFIH1 | 0.00 | 4.97 | 0.00 | 0.00 | 0.00 | 0.00 | 3.32 | 0.00 | 0.00 | 3.89 | 0.00 | 0.00 |
| SACS | 0.00 | 0.00 | 0.00 | 0.00 | 0.00 | 0.00 | 0.00 | 0.00 | 0.00 | 0.00 | 0.00 | 0.00 |
| ARMCX5 | 0.00 | 0.00 | 0.00 | 0.00 | 0.00 | 0.00 | 0.00 | 0.00 | 0.00 | 0.00 | 0.00 | 0.00 |
| C1QA | 4.15 | 5.09 | 0.00 | 0.00 | 0.00 | 0.00 | 3.30 | 0.00 | 6.72 | 0.00 | 0.00 | 0.00 |
| LGMN | 0.00 | 0.00 | 0.00 | 0.00 | 0.00 | 0.00 | 0.00 | 0.00 | 0.00 | 0.00 | 0.00 | 0.00 |
| GM4070 | 0.00 | 3.62 | 0.00 | 0.00 | 4.75 | 0.00 | 0.00 | 0.00 | 0.00 | 0.00 | 0.00 | 0.00 |
| SOS1 | 0.00 | 0.00 | 0.00 | 0.00 | 0.00 | 0.00 | 0.00 | 0.00 | 0.00 | 0.00 | 0.00 | 0.00 |
| PMEL | 0.00 | 0.00 | 0.00 | 0.00 | 0.00 | 0.00 | 0.00 | 0.00 | 0.00 | 0.00 | 0.00 | 0.00 |
| PRPF38B | 0.00 | 0.00 | 0.00 | 0.00 | 0.00 | 0.00 | 0.00 | 0.00 | 0.00 | 0.00 | 0.00 | 0.00 |
| ISG15 | 0.00 | 6.24 | 5.16 | 5.85 | 0.00 | 0.00 | 10.30 | 4.30 | 4.05 | 5.72 | 0.00 | 0.00 |
| LNPEP | 0.00 | 0.00 | 0.00 | 0.00 | 0.00 | 0.00 | 0.00 | 0.00 | 0.00 | 0.00 | 0.00 | 0.00 |
| TLN1 | 0.00 | 0.00 | 0.00 | 0.00 | 0.00 | 0.00 | 0.00 | 4.07 | 3.46 | 0.00 | 0.00 | 0.00 |
| FAM189B | 0.00 | 0.00 | 0.00 | 0.00 | 0.00 | 0.00 | 0.00 | 0.00 | 0.00 | 0.00 | 0.00 | 0.00 |
| MLH3 | 0.00 | 0.00 | 0.00 | 0.00 | 0.00 | 0.00 | 0.00 | 0.00 | 0.00 | 0.00 | 0.00 | 0.00 |
| PHF11C | 0.00 | 0.00 | 0.00 | 0.00 | 0.00 | 0.00 | 0.00 | 0.00 | 0.00 | 0.00 | 0.00 | 0.00 |
| SYNRG | 0.00 | 0.00 | 0.00 | 0.00 | 0.00 | 0.00 | 0.00 | 0.00 | 0.00 | 0.00 | 0.00 | 0.00 |
| OAS3 | 0.00 | 0.00 | 0.00 | 0.00 | 0.00 | 0.00 | 0.00 | 0.00 | 0.00 | 0.00 | 0.00 | 0.00 |
| ATP2B1 | 0.00 | 0.00 | 0.00 | 0.00 | 0.00 | 0.00 | 0.00 | 0.00 | 0.00 | 0.00 | 0.00 | 0.00 |
| TLE4 | 0.00 | 3.75 | 0.00 | 0.00 | 0.00 | 0.00 | 0.00 | 0.00 | 0.00 | 0.00 | 0.00 | 0.00 |
| ZFP217 | 0.00 | 0.00 | 0.00 | 0.00 | 0.00 | 0.00 | 0.00 | 0.00 | 0.00 | 0.00 | 0.00 | 0.00 |
| TRAF5 | 0.00 | 0.00 | 0.00 | 0.00 | 0.00 | 0.00 | 0.00 | 0.00 | 0.00 | 0.00 | 0.00 | 0.00 |
| HERPUD1 | 0.00 | 0.00 | 0.00 | 0.00 | 0.00 | 3.80 | 0.00 | 0.00 | 0.00 | 0.00 | 0.00 | 0.00 |
| GM4955 | 0.00 | 5.80 | 0.00 | 0.00 | 0.00 | 0.00 | 6.77 | 3.69 | 0.00 | 5.79 | 4.45 | 0.00 |
| WFDC17 | 0.00 | 0.00 | 0.00 | 0.00 | 0.00 | 7.39 | 4.12 | 0.00 | 0.00 | 0.00 | 0.00 | 6.73 |
| CCNT2 | 0.00 | 4.95 | 0.00 | 0.00 | 0.00 | 0.00 | 0.00 | 0.00 | 0.00 | 0.00 | 0.00 | 0.00 |
| CEP250 | 0.00 | 0.00 | 0.00 | 0.00 | 0.00 | 0.00 | 0.00 | 0.00 | 0.00 | 0.00 | 0.00 | 0.00 |
| MT-ATP8 | 0.00 | 3.11 | 0.00 | 0.00 | 3.82 | 0.00 | 3.25 | 0.00 | 4.96 | 0.00 | 0.00 | 0.00 |
| TNKS2 | 0.00 | 0.00 | 0.00 | 0.00 | 0.00 | 0.00 | 0.00 | 0.00 | 0.00 | 0.00 | 0.00 | 0.00 |
| CNOT1 | 0.00 | 0.00 | 0.00 | 0.00 | 0.00 | 0.00 | 0.00 | 0.00 | 0.00 | 0.00 | 0.00 | 0.00 |
| UBR5 | 0.00 | 0.00 | 0.00 | 0.00 | 0.00 | 0.00 | 0.00 | 0.00 | 0.00 | 0.00 | 0.00 | 0.00 |
| AGAP2 | 0.00 | 0.00 | 0.00 | 0.00 | 0.00 | 0.00 | 0.00 | 0.00 | 0.00 | 0.00 | 0.00 | 0.00 |
| CLEC12A | 0.00 | 0.00 | 0.00 | 0.00 | 0.00 | 0.00 | 0.00 | 0.00 | 0.00 | 0.00 | 0.00 | 0.00 |
| GM16586 | 0.00 | 0.00 | 0.00 | 0.00 | 0.00 | 0.00 | 0.00 | 0.00 | 0.00 | 0.00 | 0.00 | 0.00 |
| PHF14 | 0.00 | 0.00 | 0.00 | 0.00 | 0.00 | 0.00 | 0.00 | 0.00 | 0.00 | 0.00 | 0.00 | 0.00 |
| LYST | 0.00 | 0.00 | 0.00 | 0.00 | 0.00 | 0.00 | 0.00 | 0.00 | 0.00 | 0.00 | 0.00 | 0.00 |
| HIP1 | 0.00 | 0.00 | 0.00 | 0.00 | 0.00 | 0.00 | 0.00 | 0.00 | 0.00 | 0.00 | 0.00 | 0.00 |
| EML4 | 0.00 | 0.00 | 4.40 | 0.00 | 0.00 | 0.00 | 0.00 | 3.64 | 0.00 | 0.00 | 0.00 | 0.00 |
| GVIN1 | 0.00 | 0.00 | 0.00 | 0.00 | 0.00 | 0.00 | 4.04 | 0.00 | 0.00 | 0.00 | 0.00 | 0.00 |
| MADD | 0.00 | 0.00 | 0.00 | 0.00 | 0.00 | 0.00 | 0.00 | 0.00 | 0.00 | 0.00 | 0.00 | 0.00 |
| HOOK1 | 0.00 | 0.00 | 0.00 | 0.00 | 0.00 | 0.00 | 0.00 | 0.00 | 0.00 | 0.00 | 0.00 | 0.00 |
| LYZ2 | 0.00 | 0.00 | 0.00 | 5.30 | 4.36 | 4.18 | 0.00 | 0.00 | 3.43 | 0.00 | 0.00 | 0.00 |
| DPP4 | 0.00 | 0.00 | 0.00 | 0.00 | 0.00 | 0.00 | 0.00 | 0.00 | 0.00 | 0.00 | 0.00 | 0.00 |
| MYB | 0.00 | 0.00 | 0.00 | 0.00 | 0.00 | 0.00 | 0.00 | 0.00 | 0.00 | 0.00 | 0.00 | 0.00 |
| TMEM209 | 0.00 | 0.00 | 0.00 | 0.00 | 0.00 | 0.00 | 0.00 | 0.00 | 0.00 | 0.00 | 0.00 | 0.00 |
| MYO9A | 0.00 | 0.00 | 0.00 | 0.00 | 0.00 | 0.00 | 0.00 | 0.00 | 0.00 | 0.00 | 0.00 | 0.00 |
| SP3 | 0.00 | 0.00 | 0.00 | 0.00 | 0.00 | 0.00 | 0.00 | 0.00 | 0.00 | 0.00 | 0.00 | 0.00 |
| FAM208B | 0.00 | 0.00 | 0.00 | 0.00 | 0.00 | 0.00 | 0.00 | 0.00 | 0.00 | 0.00 | 0.00 | 0.00 |
| TMEM87A | 0.00 | 0.00 | 0.00 | 0.00 | 0.00 | 0.00 | 0.00 | 0.00 | 0.00 | 0.00 | 0.00 | 0.00 |
| PTPN13 | 0.00 | 0.00 | 0.00 | 0.00 | 0.00 | 0.00 | 3.07 | 0.00 | 0.00 | 0.00 | 0.00 | 0.00 |
| CDK13 | 0.00 | 0.00 | 0.00 | 0.00 | 0.00 | 0.00 | 0.00 | 0.00 | 0.00 | 0.00 | 0.00 | 0.00 |
| POLDIP3 | 0.00 | 0.00 | 0.00 | 0.00 | 0.00 | 0.00 | 0.00 | 0.00 | 0.00 | 0.00 | 0.00 | 0.00 |
| C1QC | 0.00 | 5.57 | 0.00 | 0.00 | 0.00 | 0.00 | 0.00 | 0.00 | 3.53 | 0.00 | 0.00 | 0.00 |
| MBD5 | 0.00 | 0.00 | 0.00 | 0.00 | 0.00 | 0.00 | 0.00 | 0.00 | 0.00 | 0.00 | 0.00 | 0.00 |
| IFIT1BL1 | 0.00 | 0.00 | 0.00 | 0.00 | 0.00 | 0.00 | 0.00 | 0.00 | 0.00 | 0.00 | 0.00 | 0.00 |
| ITPR2 | 0.00 | 0.00 | 0.00 | 0.00 | 0.00 | 0.00 | 0.00 | 0.00 | 0.00 | 0.00 | 0.00 | 0.00 |
| ZFP874B | 0.00 | 0.00 | 0.00 | 0.00 | 0.00 | 0.00 | 0.00 | 0.00 | 0.00 | 0.00 | 0.00 | 0.00 |
| SSH1 | 0.00 | 0.00 | 0.00 | 0.00 | 0.00 | 0.00 | 0.00 | 0.00 | 0.00 | 0.00 | 0.00 | 0.00 |
| HBA-A1 | 0.00 | 0.00 | 0.00 | 0.00 | 0.00 | 0.00 | 0.00 | 0.00 | 0.00 | 0.00 | 0.00 | 6.06 |
| STRIP1 | 0.00 | 0.00 | 0.00 | 0.00 | 0.00 | 0.00 | 0.00 | 0.00 | 0.00 | 0.00 | 0.00 | 0.00 |
| SLMAP | 0.00 | 0.00 | 0.00 | 0.00 | 0.00 | 0.00 | 0.00 | 0.00 | 0.00 | 0.00 | 0.00 | 0.00 |
| 1700020I14RIK | 0.00 | 0.00 | 0.00 | 0.00 | 0.00 | 0.00 | 0.00 | 0.00 | 0.00 | 0.00 | 0.00 | 0.00 |
| 2310040G24RIK | 0.00 | 0.00 | 0.00 | 0.00 | 0.00 | 0.00 | 0.00 | 0.00 | 3.39 | 0.00 | 0.00 | 0.00 |
| 2510046G10RIK | 0.00 | 0.00 | 0.00 | 0.00 | 0.00 | 3.90 | 0.00 | 0.00 | 0.00 | 0.00 | 0.00 | 0.00 |
| 2810474O19RIK | 0.00 | 4.36 | 0.00 | 0.00 | 0.00 | 0.00 | 0.00 | 0.00 | 0.00 | 0.00 | 0.00 | 0.00 |
| 2900060B14RIK | 0.00 | 0.00 | 0.00 | 0.00 | 0.00 | 0.00 | 0.00 | 0.00 | 3.66 | 0.00 | 0.00 | 0.00 |
| 4932438A13RIK | 0.00 | 0.00 | 0.00 | 0.00 | 0.00 | 0.00 | 0.00 | 0.00 | 0.00 | 0.00 | 0.00 | 0.00 |
| 9930111J21RIK1 | 0.00 | 0.00 | 0.00 | 0.00 | 0.00 | 0.00 | 0.00 | 0.00 | 3.58 | 3.85 | 0.00 | 0.00 |
| 9930111J21RIK2 | 0.00 | 3.32 | 0.00 | 0.00 | 0.00 | 0.00 | 0.00 | 4.21 | 4.59 | 0.00 | 0.00 | 0.00 |
| A530040E14RIK | 0.00 | 3.68 | 0.00 | 0.00 | 0.00 | 0.00 | 3.10 | 0.00 | 0.00 | 0.00 | 0.00 | 0.00 |
| A930007I19RIK | 0.00 | 0.00 | 0.00 | 0.00 | 0.00 | 0.00 | 0.00 | 0.00 | 3.64 | 0.00 | 0.00 | 0.00 |
| AA467197 | 0.00 | 0.00 | 0.00 | 0.00 | 0.00 | 0.00 | 0.00 | 0.00 | 0.00 | 0.00 | 0.00 | 3.69 |
| ABLIM1 | 0.00 | 0.00 | 0.00 | 0.00 | 0.00 | 0.00 | 3.27 | 0.00 | 0.00 | 0.00 | 0.00 | 0.00 |
| ACTB | 4.38 | 0.00 | 0.00 | 0.00 | 0.00 | 0.00 | 0.00 | 0.00 | 0.00 | 0.00 | 0.00 | 0.00 |
| ACTG1 | 0.00 | 0.00 | 0.00 | 0.00 | 5.67 | 0.00 | 0.00 | 0.00 | 0.00 | 4.29 | 0.00 | 0.00 |
| ACTR3 | 0.00 | 0.00 | 0.00 | 0.00 | 0.00 | 0.00 | 3.12 | 0.00 | 0.00 | 0.00 | 0.00 | 0.00 |
| ADAM8 | 0.00 | 0.00 | 0.00 | 0.00 | 0.00 | 0.00 | 0.00 | 0.00 | 0.00 | 0.00 | 0.00 | 3.74 |
| ADAP2 | 0.00 | 3.70 | 0.00 | 0.00 | 0.00 | 0.00 | 0.00 | 0.00 | 0.00 | 0.00 | 0.00 | 0.00 |
| ADK | 0.00 | 0.00 | 0.00 | 0.00 | 0.00 | 0.00 | 0.00 | 0.00 | 0.00 | 0.00 | 0.00 | 0.00 |
| ADPGK | 0.00 | 0.00 | 0.00 | 0.00 | 0.00 | 0.00 | 0.00 | 0.00 | 0.00 | 0.00 | 0.00 | 4.78 |
| ADRB2 | 4.02 | 0.00 | 0.00 | 0.00 | 3.78 | 0.00 | 0.00 | 0.00 | 3.37 | 0.00 | 0.00 | 0.00 |
| ADRBK2 | 0.00 | 0.00 | 0.00 | 0.00 | 0.00 | 0.00 | 3.97 | 0.00 | 0.00 | 0.00 | 0.00 | 0.00 |
| AFF1 | 0.00 | 0.00 | 0.00 | 0.00 | 0.00 | 0.00 | 4.03 | 0.00 | 0.00 | 0.00 | 0.00 | 0.00 |
| AFG3L2 | 0.00 | 0.00 | 0.00 | 0.00 | 0.00 | 0.00 | 3.45 | 0.00 | 0.00 | 0.00 | 0.00 | 0.00 |
| AHCYL2 | 0.00 | 0.00 | 0.00 | 0.00 | 0.00 | 0.00 | 0.00 | 0.00 | 0.00 | 0.00 | 0.00 | 0.00 |
| AI607873 | 0.00 | 5.86 | 4.65 | 0.00 | 0.00 | 0.00 | 0.00 | 5.28 | 0.00 | 0.00 | 0.00 | 0.00 |
| AKAP10 | 0.00 | 0.00 | 0.00 | 0.00 | 0.00 | 0.00 | 0.00 | 0.00 | 0.00 | 0.00 | 0.00 | 0.00 |
| AKAP11 | 0.00 | 0.00 | 0.00 | 0.00 | 0.00 | 0.00 | 0.00 | 0.00 | 0.00 | 0.00 | 0.00 | 0.00 |
| ANKRD33B | 0.00 | 0.00 | 0.00 | 0.00 | 0.00 | 0.00 | 0.00 | 0.00 | 0.00 | 0.00 | 0.00 | 4.22 |
| ANXA1 | 0.00 | 0.00 | 0.00 | 0.00 | 0.00 | 0.00 | 0.00 | 0.00 | 0.00 | 0.00 | 0.00 | 10.18 |
| ANXA2 | 0.00 | 0.00 | 0.00 | 0.00 | 0.00 | 0.00 | 0.00 | 0.00 | 0.00 | 0.00 | 0.00 | 0.00 |
| AOAH | 0.00 | 0.00 | 0.00 | 0.00 | 0.00 | 0.00 | 0.00 | 0.00 | 3.47 | 0.00 | 0.00 | 0.00 |
| AP2B1 | 0.00 | 0.00 | 0.00 | 0.00 | 0.00 | 0.00 | 0.00 | 0.00 | 0.00 | 0.00 | 0.00 | 0.00 |
| AP3M2 | 0.00 | 0.00 | 0.00 | 0.00 | 0.00 | 0.00 | 0.00 | 0.00 | 0.00 | 0.00 | 0.00 | 3.43 |
| APC | 0.00 | 0.00 | 0.00 | 0.00 | 0.00 | 0.00 | 0.00 | 0.00 | 0.00 | 0.00 | 0.00 | 0.00 |
| APOBR | 0.00 | 0.00 | 0.00 | 0.00 | 0.00 | 0.00 | 0.00 | 0.00 | 0.00 | 0.00 | 0.00 | 0.00 |
| APOD | 0.00 | 0.00 | 0.00 | 0.00 | 0.00 | 4.81 | 0.00 | 0.00 | 0.00 | 0.00 | 0.00 | 0.00 |
| APOE | 4.30 | 0.00 | 0.00 | 0.00 | 0.00 | 0.00 | 0.00 | 3.62 | 0.00 | 0.00 | 0.00 | 0.00 |
| APOL9B | 0.00 | 0.00 | 0.00 | 0.00 | 0.00 | 0.00 | 0.00 | 0.00 | 0.00 | 0.00 | 0.00 | 0.00 |
| ARG2 | 0.00 | 0.00 | 0.00 | 0.00 | 0.00 | 0.00 | 0.00 | 0.00 | 0.00 | 0.00 | 0.00 | 0.00 |
| ARL5A | 0.00 | 0.00 | 0.00 | 0.00 | 0.00 | 0.00 | 0.00 | 0.00 | 0.00 | 0.00 | 0.00 | 0.00 |
| ARMC7 | 0.00 | 3.45 | 0.00 | 0.00 | 0.00 | 0.00 | 0.00 | 0.00 | 0.00 | 0.00 | 0.00 | 0.00 |
| ASB13 | 0.00 | 0.00 | 0.00 | 0.00 | 0.00 | 0.00 | 3.18 | 0.00 | 0.00 | 0.00 | 0.00 | 0.00 |
| ASS1 | 0.00 | 0.00 | 0.00 | 0.00 | 0.00 | 3.89 | 0.00 | 0.00 | 0.00 | 0.00 | 0.00 | 0.00 |
| ASXL1 | 0.00 | 0.00 | 0.00 | 0.00 | 0.00 | 0.00 | 0.00 | 0.00 | 0.00 | 0.00 | 0.00 | 0.00 |
| ATF6 | 0.00 | 0.00 | 0.00 | 0.00 | 0.00 | 0.00 | 0.00 | 0.00 | 0.00 | 0.00 | 0.00 | 0.00 |
| ATF7IP | 0.00 | 0.00 | 0.00 | 0.00 | 0.00 | 0.00 | 0.00 | 0.00 | 0.00 | 0.00 | 0.00 | 0.00 |
| ATP11B | 0.00 | 0.00 | 0.00 | 0.00 | 0.00 | 0.00 | 0.00 | 0.00 | 0.00 | 0.00 | 0.00 | 0.00 |
| ATP8B4 | 0.00 | 0.00 | 0.00 | 0.00 | 0.00 | 0.00 | 0.00 | 0.00 | 0.00 | 0.00 | 0.00 | 0.00 |
| AU020206 | 0.00 | 0.00 | 0.00 | 0.00 | 0.00 | 0.00 | 0.00 | 0.00 | 0.00 | 0.00 | 0.00 | 0.00 |
| AW112010 | 0.00 | 0.00 | 0.00 | 0.00 | 0.00 | 0.00 | 0.00 | 0.00 | 0.00 | 4.30 | 0.00 | 5.35 |
| AY036118 | 0.00 | 0.00 | 0.00 | 5.42 | 0.00 | 0.00 | 0.00 | 3.97 | 0.00 | 0.00 | 0.00 | 0.00 |
| B130006D01RIK | 0.00 | 0.00 | 0.00 | 0.00 | 3.98 | 0.00 | 0.00 | 0.00 | 0.00 | 0.00 | 0.00 | 0.00 |
| B2M | 0.00 | 4.23 | 0.00 | 0.00 | 0.00 | 0.00 | 4.83 | 0.00 | 3.39 | 4.10 | 0.00 | 0.00 |
| B430306N03RIK | 0.00 | 0.00 | 0.00 | 0.00 | 0.00 | 0.00 | 3.35 | 0.00 | 0.00 | 0.00 | 0.00 | 0.00 |
| B4GALT1 | 0.00 | 0.00 | 0.00 | 0.00 | 0.00 | 0.00 | 0.00 | 0.00 | 0.00 | 0.00 | 0.00 | 3.60 |
| B4GALT4 | 0.00 | 0.00 | 0.00 | 0.00 | 0.00 | 0.00 | 3.56 | 0.00 | 0.00 | 0.00 | 0.00 | 0.00 |
| BACH2 | 0.00 | 0.00 | 0.00 | 0.00 | 0.00 | 0.00 | 0.00 | 0.00 | 0.00 | 0.00 | 0.00 | 0.00 |
| BASP1 | 0.00 | 0.00 | 0.00 | 0.00 | 0.00 | 0.00 | 0.00 | 0.00 | 0.00 | 0.00 | 0.00 | 11.53 |
| BATF2 | 0.00 | 0.00 | 0.00 | 0.00 | 0.00 | 0.00 | 0.00 | 0.00 | 4.86 | 0.00 | 0.00 | 0.00 |
| BATF3 | 0.00 | 0.00 | 0.00 | 0.00 | 0.00 | 0.00 | 0.00 | 0.00 | 4.22 | 0.00 | 0.00 | 0.00 |
| BAZ1A | 0.00 | 0.00 | 0.00 | 0.00 | 0.00 | 0.00 | 0.00 | 0.00 | 0.00 | 0.00 | 0.00 | 0.00 |
| BAZ2B | 0.00 | 0.00 | 0.00 | 0.00 | 0.00 | 0.00 | 0.00 | 0.00 | 0.00 | 0.00 | 0.00 | 0.00 |
| BBC3 | 0.00 | 0.00 | 0.00 | 0.00 | 4.17 | 0.00 | 0.00 | 0.00 | 0.00 | 0.00 | 0.00 | 0.00 |
| BC094916 | 0.00 | 0.00 | 0.00 | 0.00 | 0.00 | 0.00 | 0.00 | 0.00 | 0.00 | 0.00 | 0.00 | 0.00 |
| BC147527 | 0.00 | 7.10 | 0.00 | 0.00 | 0.00 | 0.00 | 0.00 | 0.00 | 0.00 | 0.00 | 0.00 | 0.00 |
| BCL2L2 | 0.00 | 0.00 | 0.00 | 0.00 | 4.23 | 0.00 | 0.00 | 0.00 | 0.00 | 0.00 | 0.00 | 0.00 |
| BEX6 | 0.00 | 0.00 | 0.00 | 0.00 | 0.00 | 0.00 | 3.51 | 0.00 | 0.00 | 0.00 | 0.00 | 0.00 |
| BLVRB | 0.00 | 0.00 | 0.00 | 0.00 | 0.00 | 0.00 | 0.00 | 0.00 | 3.80 | 0.00 | 0.00 | 0.00 |
| BMYC | 0.00 | 0.00 | 0.00 | 0.00 | 0.00 | 0.00 | 0.00 | 0.00 | 0.00 | 0.00 | 0.00 | 0.00 |
| BNIP3 | 0.00 | 0.00 | 0.00 | 0.00 | 0.00 | 0.00 | 0.00 | 5.55 | 0.00 | 0.00 | 0.00 | 6.46 |
| BNIP3L | 0.00 | 0.00 | 0.00 | 0.00 | 0.00 | 0.00 | 0.00 | 0.00 | 3.38 | 0.00 | 0.00 | 0.00 |
| BRD3 | 0.00 | 0.00 | 0.00 | 0.00 | 3.70 | 0.00 | 0.00 | 0.00 | 0.00 | 0.00 | 0.00 | 0.00 |
| BST1 | 0.00 | 0.00 | 0.00 | 0.00 | 0.00 | 0.00 | 0.00 | 0.00 | 0.00 | 0.00 | 0.00 | 3.78 |
| BST2 | 0.00 | 3.90 | 0.00 | 0.00 | 4.20 | 0.00 | 3.23 | 0.00 | 3.86 | 0.00 | 0.00 | 0.00 |
| BTG2 | 0.00 | 0.00 | 0.00 | 0.00 | 0.00 | 0.00 | 3.14 | 0.00 | 0.00 | 0.00 | 0.00 | 3.26 |
| C130026I21RIK | 0.00 | 4.76 | 0.00 | 0.00 | 0.00 | 0.00 | 5.23 | 0.00 | 0.00 | 4.25 | 0.00 | 0.00 |
| C5AR1 | 0.00 | 0.00 | 0.00 | 0.00 | 0.00 | 0.00 | 0.00 | 0.00 | 0.00 | 0.00 | 0.00 | 3.84 |
| CALM1 | 0.00 | 0.00 | 5.37 | 0.00 | 0.00 | 0.00 | 0.00 | 0.00 | 0.00 | 0.00 | 0.00 | 0.00 |
| CAPNS1 | 0.00 | 0.00 | 0.00 | 0.00 | 0.00 | 0.00 | 0.00 | 0.00 | 0.00 | 0.00 | 0.00 | 0.00 |
| CAR14 | 0.00 | 0.00 | 0.00 | 0.00 | 0.00 | 0.00 | 0.00 | 0.00 | 0.00 | 0.00 | 0.00 | 0.00 |
| CARD19 | 0.00 | 0.00 | 0.00 | 0.00 | 0.00 | 0.00 | 0.00 | 0.00 | 0.00 | 0.00 | 0.00 | 10.50 |
| CASC5 | 0.00 | 0.00 | 0.00 | 0.00 | 0.00 | 0.00 | 0.00 | 0.00 | 0.00 | 0.00 | 0.00 | 0.00 |
| CASP4 | 0.00 | 3.46 | 0.00 | 0.00 | 0.00 | 0.00 | 3.81 | 0.00 | 0.00 | 0.00 | 0.00 | 0.00 |
| CASP8AP2 | 0.00 | 0.00 | 0.00 | 0.00 | 0.00 | 0.00 | 0.00 | 0.00 | 0.00 | 0.00 | 0.00 | 0.00 |
| CBX5 | 0.00 | 0.00 | 0.00 | 0.00 | 0.00 | 0.00 | 0.00 | 0.00 | 0.00 | 0.00 | 0.00 | 0.00 |
| CCDC141 | 0.00 | 0.00 | 0.00 | 0.00 | 0.00 | 0.00 | 0.00 | 0.00 | 0.00 | 0.00 | 0.00 | 0.00 |
| CCDC162 | 0.00 | 0.00 | 0.00 | 0.00 | 0.00 | 0.00 | 0.00 | 0.00 | 0.00 | 0.00 | 0.00 | 0.00 |
| CCDC88A | 0.00 | 0.00 | 0.00 | 0.00 | 0.00 | 0.00 | 3.21 | 0.00 | 0.00 | 0.00 | 0.00 | 0.00 |
| CCDC88C | 0.00 | 0.00 | 0.00 | 0.00 | 0.00 | 0.00 | 0.00 | 0.00 | 0.00 | 0.00 | 0.00 | 0.00 |
| CCDC94 | 0.00 | 3.18 | 0.00 | 0.00 | 0.00 | 0.00 | 0.00 | 0.00 | 0.00 | 0.00 | 0.00 | 0.00 |
| CCL1 | 0.00 | 0.00 | 0.00 | 0.00 | 6.10 | 0.00 | 0.00 | 0.00 | 0.00 | 0.00 | 0.00 | 0.00 |
| CCL12 | 0.00 | 0.00 | 0.00 | 0.00 | 0.00 | 0.00 | 0.00 | 0.00 | 3.30 | 0.00 | 0.00 | 0.00 |
| CCL2 | 0.00 | 0.00 | 0.00 | 0.00 | 0.00 | 0.00 | 0.00 | 0.00 | 0.00 | 0.00 | 0.00 | 0.00 |
| CCL24 | 0.00 | 0.00 | 0.00 | 0.00 | 0.00 | 0.00 | 0.00 | 0.00 | 3.18 | 0.00 | 0.00 | 0.00 |
| CCL3 | 0.00 | 0.00 | 0.00 | 0.00 | 0.00 | 0.00 | 0.00 | 5.52 | 0.00 | 0.00 | 0.00 | 26.34 |
| CCL4 | 0.00 | 0.00 | 0.00 | 0.00 | 0.00 | 0.00 | 0.00 | 0.00 | 0.00 | 0.00 | 0.00 | 26.59 |
| CCL5 | 0.00 | 0.00 | 0.00 | 0.00 | 0.00 | 0.00 | 0.00 | 0.00 | 0.00 | 4.57 | 0.00 | 0.00 |
| CCL7 | 0.00 | 3.54 | 0.00 | 0.00 | 3.89 | 7.63 | 0.00 | 0.00 | 0.00 | 0.00 | 0.00 | 0.00 |
| CCL9 | 0.00 | 0.00 | 4.90 | 0.00 | 0.00 | 0.00 | 0.00 | 0.00 | 0.00 | 0.00 | 0.00 | 0.00 |
| CCNB1 | 0.00 | 0.00 | 0.00 | 0.00 | 0.00 | 0.00 | 4.21 | 0.00 | 0.00 | 0.00 | 0.00 | 0.00 |
| CCND1 | 0.00 | 0.00 | 0.00 | 0.00 | 0.00 | 0.00 | 5.36 | 0.00 | 0.00 | 0.00 | 0.00 | 0.00 |
| CCND2 | 0.00 | 0.00 | 0.00 | 0.00 | 0.00 | 0.00 | 3.03 | 0.00 | 0.00 | 3.87 | 0.00 | 0.00 |
| CCNE2 | 3.69 | 0.00 | 0.00 | 0.00 | 0.00 | 0.00 | 0.00 | 0.00 | 0.00 | 0.00 | 0.00 | 0.00 |
| CCNG2 | 0.00 | 0.00 | 0.00 | 0.00 | 0.00 | 0.00 | 0.00 | 0.00 | 3.20 | 0.00 | 0.00 | 0.00 |
| CCNL1 | 3.94 | 0.00 | 0.00 | 0.00 | 0.00 | 0.00 | 0.00 | 0.00 | 0.00 | 0.00 | 0.00 | 0.00 |
| CCR1 | 0.00 | 0.00 | 0.00 | 0.00 | 0.00 | 0.00 | 0.00 | 0.00 | 0.00 | 0.00 | 0.00 | 11.89 |
| CCR2 | 0.00 | 3.93 | 0.00 | 0.00 | 0.00 | 0.00 | 0.00 | 4.72 | 0.00 | 0.00 | 0.00 | 0.00 |
| CCR3 | 0.00 | 0.00 | 0.00 | 0.00 | 0.00 | 0.00 | 0.00 | 0.00 | 0.00 | 0.00 | 0.00 | 0.00 |
| CCR7 | 0.00 | 0.00 | 0.00 | 0.00 | 0.00 | 0.00 | 0.00 | 0.00 | 0.00 | 0.00 | 0.00 | 0.00 |
| CCRL2 | 0.00 | 0.00 | 0.00 | 0.00 | 0.00 | 0.00 | 0.00 | 0.00 | 0.00 | 0.00 | 0.00 | 4.90 |
| CD14 | 0.00 | 0.00 | 0.00 | 5.01 | 0.00 | 0.00 | 0.00 | 0.00 | 0.00 | 0.00 | 0.00 | 12.47 |
| CD160 | 0.00 | 0.00 | 0.00 | 0.00 | 0.00 | 0.00 | 0.00 | 0.00 | 0.00 | 0.00 | 0.00 | 0.00 |
| CD177 | 0.00 | 0.00 | 0.00 | 0.00 | 0.00 | 0.00 | 0.00 | 0.00 | 0.00 | 0.00 | 0.00 | 3.29 |
| CD180 | 0.00 | 0.00 | 0.00 | 0.00 | 0.00 | 0.00 | 0.00 | 0.00 | 3.33 | 0.00 | 0.00 | 0.00 |
| CD2 | 0.00 | 0.00 | 0.00 | 0.00 | 0.00 | 0.00 | 0.00 | 4.12 | 0.00 | 0.00 | 0.00 | 0.00 |
| CD200 | 0.00 | 0.00 | 0.00 | 0.00 | 0.00 | 0.00 | 0.00 | 0.00 | 0.00 | 0.00 | 0.00 | 0.00 |
| CD209A | 0.00 | 0.00 | 0.00 | 0.00 | 0.00 | 0.00 | 0.00 | 4.32 | 0.00 | 0.00 | 0.00 | 0.00 |
| CD226 | 0.00 | 3.21 | 0.00 | 0.00 | 0.00 | 0.00 | 0.00 | 0.00 | 0.00 | 0.00 | 0.00 | 0.00 |
| CD24A | 0.00 | 4.05 | 0.00 | 0.00 | 0.00 | 0.00 | 0.00 | 0.00 | 0.00 | 0.00 | 0.00 | 13.24 |
| CD274 | 0.00 | 0.00 | 0.00 | 0.00 | 0.00 | 0.00 | 0.00 | 0.00 | 0.00 | 0.00 | 0.00 | 0.00 |
| CD28 | 0.00 | 0.00 | 0.00 | 0.00 | 0.00 | 0.00 | 0.00 | 11.86 | 0.00 | 3.87 | 0.00 | 0.00 |
| CD2AP | 0.00 | 0.00 | 0.00 | 0.00 | 0.00 | 0.00 | 3.55 | 4.07 | 0.00 | 0.00 | 0.00 | 0.00 |
| CD300LD | 0.00 | 0.00 | 0.00 | 0.00 | 0.00 | 0.00 | 0.00 | 0.00 | 3.22 | 0.00 | 0.00 | 0.00 |
| CD300LF | 0.00 | 0.00 | 0.00 | 0.00 | 0.00 | 0.00 | 0.00 | 0.00 | 0.00 | 0.00 | 0.00 | 3.88 |
| CD4 | 0.00 | 0.00 | 0.00 | 0.00 | 0.00 | 0.00 | 0.00 | 0.00 | 0.00 | 0.00 | 0.00 | 0.00 |
| CD40 | 0.00 | 3.80 | 0.00 | 0.00 | 0.00 | 0.00 | 6.73 | 0.00 | 0.00 | 0.00 | 0.00 | 0.00 |
| CD52 | 0.00 | 0.00 | 0.00 | 0.00 | 4.60 | 0.00 | 0.00 | 0.00 | 0.00 | 0.00 | 0.00 | 0.00 |
| CD55 | 0.00 | 0.00 | 0.00 | 0.00 | 0.00 | 0.00 | 0.00 | 0.00 | 0.00 | 0.00 | 0.00 | 3.51 |
| CD63 | 0.00 | 0.00 | 0.00 | 0.00 | 0.00 | 0.00 | 0.00 | 0.00 | 0.00 | 0.00 | 0.00 | 6.86 |
| CD74 | 0.00 | 4.81 | 0.00 | 0.00 | 6.35 | 0.00 | 0.00 | 4.74 | 8.85 | 0.00 | 6.20 | 0.00 |
| CD79A | 0.00 | 0.00 | 0.00 | 0.00 | 0.00 | 0.00 | 0.00 | 0.00 | 0.00 | 0.00 | 0.00 | 4.18 |
| CD79B | 0.00 | 0.00 | 0.00 | 0.00 | 0.00 | 0.00 | 0.00 | 0.00 | 0.00 | 0.00 | 0.00 | 0.00 |
| CD81 | 0.00 | 0.00 | 0.00 | 0.00 | 0.00 | 0.00 | 3.47 | 0.00 | 0.00 | 0.00 | 0.00 | 0.00 |
| CD83 | 10.35 | 0.00 | 0.00 | 0.00 | 0.00 | 0.00 | 0.00 | 0.00 | 0.00 | 0.00 | 0.00 | 0.00 |
| CD86 | 0.00 | 0.00 | 0.00 | 0.00 | 0.00 | 0.00 | 0.00 | 0.00 | 0.00 | 0.00 | 0.00 | 0.00 |
| CD9 | 0.00 | 5.73 | 0.00 | 0.00 | 0.00 | 0.00 | 0.00 | 0.00 | 3.39 | 0.00 | 4.55 | 6.30 |
| CDC14A | 0.00 | 0.00 | 0.00 | 0.00 | 0.00 | 0.00 | 0.00 | 0.00 | 0.00 | 0.00 | 0.00 | 0.00 |
| CDKN2C | 0.00 | 0.00 | 0.00 | 0.00 | 0.00 | 0.00 | 3.20 | 0.00 | 0.00 | 0.00 | 0.00 | 0.00 |
| CEACAM1 | 0.00 | 0.00 | 0.00 | 0.00 | 0.00 | 0.00 | 0.00 | 3.76 | 0.00 | 0.00 | 0.00 | 0.00 |
| CEBPB | 5.17 | 0.00 | 0.00 | 0.00 | 0.00 | 0.00 | 0.00 | 0.00 | 0.00 | 0.00 | 0.00 | 4.29 |
| CENPH | 0.00 | 0.00 | 0.00 | 0.00 | 0.00 | 0.00 | 4.01 | 0.00 | 0.00 | 0.00 | 0.00 | 0.00 |
| CEP170 | 0.00 | 0.00 | 0.00 | 0.00 | 0.00 | 0.00 | 0.00 | 0.00 | 0.00 | 0.00 | 0.00 | 0.00 |
| CEP350 | 0.00 | 0.00 | 0.00 | 0.00 | 0.00 | 0.00 | 3.24 | 0.00 | 0.00 | 0.00 | 0.00 | 0.00 |
| CERS4 | 0.00 | 0.00 | 0.00 | 0.00 | 0.00 | 0.00 | 0.00 | 0.00 | 0.00 | 0.00 | 0.00 | 0.00 |
| CFB | 0.00 | 4.97 | 0.00 | 0.00 | 0.00 | 0.00 | 0.00 | 0.00 | 10.71 | 0.00 | 0.00 | 0.00 |
| CFH | 0.00 | 0.00 | 0.00 | 0.00 | 0.00 | 0.00 | 4.39 | 0.00 | 0.00 | 0.00 | 0.00 | 0.00 |
| CHURC1 | 0.00 | 0.00 | 0.00 | 0.00 | 0.00 | 0.00 | 0.00 | 0.00 | 0.00 | 0.00 | 0.00 | 0.00 |
| CKB | 0.00 | 0.00 | 0.00 | 0.00 | 0.00 | 0.00 | 0.00 | 0.00 | 3.94 | 0.00 | 0.00 | 0.00 |
| CLDND1 | 0.00 | 0.00 | 0.00 | 0.00 | 0.00 | 0.00 | 3.79 | 0.00 | 0.00 | 0.00 | 0.00 | 0.00 |
| CLEC2D | 0.00 | 0.00 | 0.00 | 0.00 | 0.00 | 0.00 | 3.77 | 0.00 | 5.52 | 0.00 | 0.00 | 0.00 |
| CLEC4D | 0.00 | 0.00 | 0.00 | 0.00 | 0.00 | 0.00 | 0.00 | 0.00 | 0.00 | 0.00 | 0.00 | 9.40 |
| CLEC4E | 0.00 | 0.00 | 0.00 | 0.00 | 0.00 | 0.00 | 0.00 | 0.00 | 0.00 | 0.00 | 0.00 | 4.85 |
| CLEC4N | 0.00 | 0.00 | 0.00 | 0.00 | 0.00 | 4.09 | 0.00 | 0.00 | 0.00 | 0.00 | 0.00 | 3.50 |
| CLIC4 | 0.00 | 0.00 | 0.00 | 0.00 | 0.00 | 0.00 | 3.11 | 0.00 | 0.00 | 0.00 | 0.00 | 0.00 |
| CLK1 | 0.00 | 0.00 | 0.00 | 0.00 | 0.00 | 0.00 | 0.00 | 0.00 | 0.00 | 0.00 | 0.00 | 0.00 |
| CMPK2 | 0.00 | 3.92 | 0.00 | 0.00 | 0.00 | 0.00 | 0.00 | 0.00 | 0.00 | 0.00 | 0.00 | 0.00 |
| CMTR1 | 0.00 | 0.00 | 0.00 | 0.00 | 0.00 | 0.00 | 0.00 | 0.00 | 0.00 | 0.00 | 0.00 | 0.00 |
| CNP | 0.00 | 3.34 | 0.00 | 0.00 | 0.00 | 0.00 | 0.00 | 0.00 | 0.00 | 0.00 | 0.00 | 0.00 |
| COL4A3BP | 0.00 | 0.00 | 0.00 | 0.00 | 0.00 | 0.00 | 0.00 | 0.00 | 0.00 | 0.00 | 0.00 | 0.00 |
| COX6A2 | 0.00 | 0.00 | 4.89 | 0.00 | 0.00 | 0.00 | 0.00 | 0.00 | 0.00 | 0.00 | 0.00 | 0.00 |
| CPSF3L | 0.00 | 0.00 | 0.00 | 0.00 | 0.00 | 0.00 | 0.00 | 0.00 | 0.00 | 0.00 | 0.00 | 0.00 |
| CRIP1 | 0.00 | 0.00 | 0.00 | 0.00 | 0.00 | 0.00 | 3.47 | 0.00 | 0.00 | 0.00 | 0.00 | 0.00 |
| CRLF3 | 0.00 | 0.00 | 0.00 | 0.00 | 0.00 | 0.00 | 0.00 | 0.00 | 0.00 | 0.00 | 0.00 | 0.00 |
| CRYBB1 | 0.00 | 0.00 | 0.00 | 0.00 | 0.00 | 0.00 | 0.00 | 0.00 | 3.27 | 0.00 | 0.00 | 0.00 |
| CSRNP1 | 0.00 | 0.00 | 0.00 | 0.00 | 0.00 | 0.00 | 0.00 | 0.00 | 0.00 | 0.00 | 0.00 | 4.74 |
| CSTB | 0.00 | 0.00 | 0.00 | 0.00 | 0.00 | 0.00 | 0.00 | 0.00 | 0.00 | 0.00 | 0.00 | 8.09 |
| CTLA2A | 0.00 | 0.00 | 0.00 | 0.00 | 0.00 | 0.00 | 0.00 | 0.00 | 0.00 | 3.55 | 0.00 | 0.00 |
| CTLA4 | 0.00 | 0.00 | 0.00 | 0.00 | 0.00 | 0.00 | 0.00 | 0.00 | 0.00 | 0.00 | 0.00 | 0.00 |
| CTSS | 0.00 | 5.53 | 0.00 | 0.00 | 0.00 | 0.00 | 4.64 | 0.00 | 4.71 | 0.00 | 0.00 | 0.00 |
| CTSW | 0.00 | 0.00 | 0.00 | 0.00 | 0.00 | 0.00 | 0.00 | 0.00 | 0.00 | 0.00 | 0.00 | 0.00 |
| CXCL10 | 0.00 | 3.86 | 0.00 | 0.00 | 0.00 | 0.00 | 6.95 | 0.00 | 0.00 | 0.00 | 0.00 | 0.00 |
| CXCL2 | 4.69 | 0.00 | 0.00 | 0.00 | 0.00 | 17.41 | 0.00 | 6.61 | 0.00 | 0.00 | 0.00 | 73.78 |
| CXCL9 | 0.00 | 0.00 | 0.00 | 0.00 | 0.00 | 0.00 | 0.00 | 0.00 | 0.00 | 3.76 | 0.00 | 0.00 |
| CXCR3 | 0.00 | 0.00 | 0.00 | 0.00 | 0.00 | 0.00 | 0.00 | 0.00 | 0.00 | 0.00 | 0.00 | 0.00 |
| CXCR4 | 0.00 | 0.00 | 0.00 | 0.00 | 0.00 | 0.00 | 0.00 | 0.00 | 0.00 | 0.00 | 0.00 | 3.67 |
| CYBB | 0.00 | 3.91 | 0.00 | 0.00 | 0.00 | 0.00 | 3.25 | 0.00 | 3.71 | 0.00 | 0.00 | 0.00 |
| CYCS | 0.00 | 0.00 | 0.00 | 0.00 | 0.00 | 0.00 | 3.58 | 0.00 | 0.00 | 0.00 | 0.00 | 0.00 |
| CYFIP1 | 0.00 | 4.47 | 0.00 | 0.00 | 0.00 | 0.00 | 0.00 | 0.00 | 5.35 | 0.00 | 0.00 | 0.00 |
| CYTH4 | 0.00 | 0.00 | 0.00 | 0.00 | 0.00 | 0.00 | 0.00 | 4.22 | 0.00 | 0.00 | 0.00 | 0.00 |
| D030056L22RIK | 0.00 | 0.00 | 0.00 | 0.00 | 0.00 | 0.00 | 0.00 | 0.00 | 0.00 | 0.00 | 0.00 | 0.00 |
| D13ERTD608E | 0.00 | 0.00 | 0.00 | 0.00 | 0.00 | 0.00 | 0.00 | 0.00 | 0.00 | 0.00 | 0.00 | 0.00 |
| D17WSU92E | 0.00 | 3.46 | 0.00 | 0.00 | 0.00 | 0.00 | 0.00 | 0.00 | 0.00 | 0.00 | 0.00 | 0.00 |
| D1ERTD622E | 0.00 | 0.00 | 0.00 | 0.00 | 0.00 | 0.00 | 0.00 | 0.00 | 0.00 | 0.00 | 0.00 | 0.00 |
| D930015E06RIK | 0.00 | 0.00 | 0.00 | 0.00 | 0.00 | 0.00 | 0.00 | 0.00 | 0.00 | 0.00 | 0.00 | 0.00 |
| DAXX | 0.00 | 0.00 | 0.00 | 0.00 | 0.00 | 0.00 | 3.62 | 0.00 | 0.00 | 0.00 | 0.00 | 0.00 |
| DCP1B | 0.00 | 0.00 | 0.00 | 0.00 | 0.00 | 0.00 | 0.00 | 0.00 | 0.00 | 0.00 | 4.13 | 0.00 |
| DDIT3 | 0.00 | 0.00 | 0.00 | 0.00 | 0.00 | 0.00 | 0.00 | 0.00 | 0.00 | 0.00 | 0.00 | 3.70 |
| DDIT4 | 0.00 | 0.00 | 0.00 | 0.00 | 0.00 | 0.00 | 0.00 | 0.00 | 0.00 | 3.81 | 0.00 | 0.00 |
| DDX46 | 0.00 | 0.00 | 0.00 | 0.00 | 0.00 | 0.00 | 0.00 | 0.00 | 0.00 | 0.00 | 0.00 | 0.00 |
| DDX58 | 0.00 | 4.85 | 0.00 | 0.00 | 0.00 | 0.00 | 0.00 | 0.00 | 0.00 | 0.00 | 0.00 | 0.00 |
| DEDD2 | 0.00 | 3.04 | 0.00 | 0.00 | 0.00 | 0.00 | 0.00 | 0.00 | 0.00 | 0.00 | 0.00 | 0.00 |
| DENND1B | 0.00 | 0.00 | 0.00 | 0.00 | 0.00 | 0.00 | 0.00 | 0.00 | 0.00 | 0.00 | 0.00 | 0.00 |
| DGAT1 | 0.00 | 0.00 | 0.00 | 0.00 | 0.00 | 0.00 | 0.00 | 0.00 | 0.00 | 0.00 | 0.00 | 0.00 |
| DGAT2 | 0.00 | 0.00 | 0.00 | 0.00 | 0.00 | 0.00 | 0.00 | 0.00 | 3.99 | 0.00 | 0.00 | 0.00 |
| DGCR8 | 0.00 | 0.00 | 0.00 | 0.00 | 0.00 | 0.00 | 0.00 | 0.00 | 0.00 | 0.00 | 0.00 | 0.00 |
| DHX29 | 0.00 | 0.00 | 0.00 | 0.00 | 0.00 | 0.00 | 0.00 | 0.00 | 0.00 | 0.00 | 0.00 | 0.00 |
| DHX58 | 0.00 | 0.00 | 0.00 | 0.00 | 0.00 | 0.00 | 3.70 | 0.00 | 0.00 | 0.00 | 0.00 | 0.00 |
| DHX9 | 3.59 | 0.00 | 0.00 | 0.00 | 0.00 | 0.00 | 0.00 | 0.00 | 0.00 | 0.00 | 0.00 | 0.00 |
| DIAPH1 | 0.00 | 3.30 | 0.00 | 0.00 | 0.00 | 0.00 | 0.00 | 0.00 | 0.00 | 0.00 | 0.00 | 0.00 |
| DIP2B | 0.00 | 0.00 | 0.00 | 0.00 | 0.00 | 0.00 | 0.00 | 0.00 | 0.00 | 0.00 | 0.00 | 0.00 |
| DKKL1 | 0.00 | 0.00 | 0.00 | 0.00 | 0.00 | 0.00 | 0.00 | 0.00 | 0.00 | 0.00 | 0.00 | 0.00 |
| DLG1 | 0.00 | 0.00 | 0.00 | 0.00 | 0.00 | 0.00 | 0.00 | 0.00 | 0.00 | 0.00 | 0.00 | 0.00 |
| DNAJB1 | 13.95 | 0.00 | 0.00 | 0.00 | 0.00 | 0.00 | 0.00 | 0.00 | 0.00 | 0.00 | 0.00 | 0.00 |
| DNAJC13 | 0.00 | 0.00 | 0.00 | 0.00 | 0.00 | 0.00 | 0.00 | 0.00 | 0.00 | 0.00 | 0.00 | 0.00 |
| DOPEY2 | 0.00 | 0.00 | 0.00 | 0.00 | 0.00 | 0.00 | 0.00 | 0.00 | 3.42 | 0.00 | 0.00 | 0.00 |
| DTX3L | 0.00 | 4.03 | 0.00 | 0.00 | 0.00 | 0.00 | 0.00 | 0.00 | 0.00 | 0.00 | 5.02 | 0.00 |
| DUSP1 | 5.29 | 0.00 | 0.00 | 0.00 | 0.00 | 0.00 | 0.00 | 0.00 | 0.00 | 0.00 | 0.00 | 5.66 |
| DUSP2 | 0.00 | 0.00 | 0.00 | 0.00 | 0.00 | 0.00 | 0.00 | 0.00 | 0.00 | 0.00 | 0.00 | 0.00 |
| DUSP7 | 0.00 | 0.00 | 0.00 | 0.00 | 4.18 | 0.00 | 0.00 | 0.00 | 0.00 | 0.00 | 0.00 | 0.00 |
| E130311K13RIK | 0.00 | 3.45 | 0.00 | 0.00 | 0.00 | 0.00 | 0.00 | 0.00 | 0.00 | 0.00 | 0.00 | 0.00 |
| EBF1 | 0.00 | 0.00 | 0.00 | 0.00 | 0.00 | 0.00 | 0.00 | 0.00 | 0.00 | 0.00 | 0.00 | 4.42 |
| ECE1 | 0.00 | 0.00 | 0.00 | 0.00 | 0.00 | 0.00 | 4.62 | 0.00 | 0.00 | 0.00 | 0.00 | 0.00 |
| EEA1 | 0.00 | 0.00 | 0.00 | 0.00 | 0.00 | 0.00 | 0.00 | 0.00 | 0.00 | 0.00 | 0.00 | 0.00 |
| EED | 0.00 | 0.00 | 0.00 | 0.00 | 0.00 | 0.00 | 0.00 | 0.00 | 0.00 | 0.00 | 0.00 | 0.00 |
| EEF1A1 | 0.00 | 0.00 | 0.00 | 0.00 | 3.88 | 0.00 | 0.00 | 0.00 | 0.00 | 0.00 | 0.00 | 0.00 |
| EGLN3 | 0.00 | 0.00 | 0.00 | 0.00 | 0.00 | 0.00 | 0.00 | 0.00 | 0.00 | 0.00 | 0.00 | 5.74 |
| EGR1 | 0.00 | 0.00 | 0.00 | 0.00 | 0.00 | 0.00 | 0.00 | 0.00 | 0.00 | 0.00 | 0.00 | 12.59 |
| EIF2AK2 | 0.00 | 3.69 | 0.00 | 0.00 | 0.00 | 0.00 | 0.00 | 0.00 | 0.00 | 0.00 | 0.00 | 0.00 |
| EIF4E3 | 0.00 | 0.00 | 0.00 | 0.00 | 0.00 | 0.00 | 0.00 | 0.00 | 0.00 | 0.00 | 0.00 | 0.00 |
| EMC1 | 0.00 | 0.00 | 0.00 | 0.00 | 0.00 | 0.00 | 0.00 | 0.00 | 3.67 | 0.00 | 0.00 | 0.00 |
| EOMES | 0.00 | 0.00 | 0.00 | 0.00 | 0.00 | 0.00 | 0.00 | 0.00 | 0.00 | 0.00 | 0.00 | 0.00 |
| EP300 | 0.00 | 0.00 | 0.00 | 0.00 | 0.00 | 0.00 | 0.00 | 0.00 | 0.00 | 0.00 | 0.00 | 0.00 |
| EPSTI1 | 0.00 | 3.05 | 0.00 | 0.00 | 0.00 | 0.00 | 0.00 | 0.00 | 3.46 | 0.00 | 0.00 | 0.00 |
| ERBB2IP | 0.00 | 0.00 | 0.00 | 0.00 | 0.00 | 0.00 | 2.98 | 0.00 | 0.00 | 0.00 | 0.00 | 0.00 |
| ERDR1 | 4.26 | 0.00 | 0.00 | 0.00 | 0.00 | 4.24 | 0.00 | 0.00 | 0.00 | 0.00 | 0.00 | 0.00 |
| ERGIC2 | 0.00 | 0.00 | 0.00 | 0.00 | 0.00 | 0.00 | 0.00 | 0.00 | 0.00 | 0.00 | 0.00 | 3.44 |
| ERO1L | 0.00 | 0.00 | 0.00 | 0.00 | 0.00 | 0.00 | 0.00 | 0.00 | 0.00 | 0.00 | 0.00 | 8.90 |
| ETS1 | 0.00 | 0.00 | 0.00 | 0.00 | 0.00 | 0.00 | 0.00 | 0.00 | 0.00 | 8.26 | 0.00 | 0.00 |
| ETS2 | 0.00 | 0.00 | 0.00 | 0.00 | 0.00 | 0.00 | 0.00 | 0.00 | 0.00 | 0.00 | 0.00 | 4.60 |
| ETV3 | 0.00 | 0.00 | 0.00 | 0.00 | 0.00 | 0.00 | 5.51 | 0.00 | 0.00 | 0.00 | 0.00 | 0.00 |
| EVI2A | 0.00 | 0.00 | 0.00 | 0.00 | 0.00 | 0.00 | 3.25 | 0.00 | 0.00 | 0.00 | 0.00 | 0.00 |
| EVI5L | 0.00 | 0.00 | 0.00 | 0.00 | 0.00 | 0.00 | 0.00 | 0.00 | 0.00 | 0.00 | 0.00 | 0.00 |
| FAM107B | 3.89 | 0.00 | 0.00 | 0.00 | 0.00 | 0.00 | 0.00 | 0.00 | 0.00 | 0.00 | 0.00 | 0.00 |
| FAM111A | 0.00 | 0.00 | 0.00 | 0.00 | 0.00 | 0.00 | 5.20 | 0.00 | 0.00 | 0.00 | 0.00 | 0.00 |
| FAM162A | 0.00 | 0.00 | 0.00 | 0.00 | 0.00 | 0.00 | 0.00 | 0.00 | 0.00 | 0.00 | 0.00 | 0.00 |
| FAM26F | 0.00 | 0.00 | 0.00 | 0.00 | 0.00 | 0.00 | 3.13 | 0.00 | 0.00 | 4.33 | 0.00 | 0.00 |
| FAM49A | 0.00 | 0.00 | 0.00 | 0.00 | 0.00 | 0.00 | 4.69 | 0.00 | 0.00 | 0.00 | 0.00 | 0.00 |
| FAM76B | 0.00 | 0.00 | 0.00 | 0.00 | 0.00 | 0.00 | 0.00 | 0.00 | 3.18 | 0.00 | 0.00 | 0.00 |
| FBXL3 | 0.00 | 0.00 | 0.00 | 0.00 | 0.00 | 0.00 | 0.00 | 0.00 | 0.00 | 0.00 | 0.00 | 0.00 |
| FCER1G | 0.00 | 0.00 | 0.00 | 0.00 | 0.00 | 0.00 | 4.66 | 0.00 | 0.00 | 0.00 | 0.00 | 0.00 |
| FCGR1 | 0.00 | 3.53 | 0.00 | 0.00 | 0.00 | 0.00 | 6.07 | 0.00 | 0.00 | 0.00 | 0.00 | 0.00 |
| FCGR3 | 0.00 | 0.00 | 0.00 | 0.00 | 0.00 | 0.00 | 0.00 | 4.18 | 0.00 | 0.00 | 0.00 | 0.00 |
| FCGR4 | 0.00 | 4.62 | 0.00 | 0.00 | 0.00 | 0.00 | 3.23 | 0.00 | 3.73 | 0.00 | 0.00 | 0.00 |
| FCMR | 0.00 | 0.00 | 0.00 | 0.00 | 0.00 | 0.00 | 0.00 | 0.00 | 0.00 | 0.00 | 0.00 | 0.00 |
| FGL2 | 0.00 | 4.95 | 0.00 | 0.00 | 0.00 | 0.00 | 5.28 | 0.00 | 0.00 | 0.00 | 0.00 | 0.00 |
| FIG4 | 0.00 | 0.00 | 0.00 | 0.00 | 0.00 | 0.00 | 0.00 | 0.00 | 0.00 | 0.00 | 4.15 | 0.00 |
| FILIP1L | 0.00 | 3.80 | 0.00 | 0.00 | 0.00 | 0.00 | 0.00 | 0.00 | 0.00 | 0.00 | 0.00 | 0.00 |
| FMNL2 | 0.00 | 0.00 | 0.00 | 0.00 | 0.00 | 0.00 | 0.00 | 0.00 | 0.00 | 0.00 | 0.00 | 3.34 |
| FMR1 | 0.00 | 0.00 | 0.00 | 0.00 | 0.00 | 0.00 | 0.00 | 0.00 | 0.00 | 0.00 | 0.00 | 0.00 |
| FOSB | 3.53 | 0.00 | 0.00 | 0.00 | 0.00 | 0.00 | 0.00 | 0.00 | 0.00 | 0.00 | 0.00 | 0.00 |
| FOXP3 | 0.00 | 0.00 | 0.00 | 0.00 | 0.00 | 0.00 | 0.00 | 0.00 | 0.00 | 0.00 | 0.00 | 0.00 |
| FPR2 | 0.00 | 0.00 | 0.00 | 0.00 | 0.00 | 0.00 | 0.00 | 0.00 | 0.00 | 0.00 | 0.00 | 4.28 |
| FRAT2 | 0.00 | 0.00 | 0.00 | 0.00 | 3.83 | 0.00 | 0.00 | 0.00 | 0.00 | 0.00 | 0.00 | 0.00 |
| FRMD4A | 0.00 | 5.43 | 0.00 | 0.00 | 0.00 | 0.00 | 0.00 | 0.00 | 0.00 | 0.00 | 0.00 | 0.00 |
| FTH1 | 0.00 | 0.00 | 0.00 | 0.00 | 0.00 | 0.00 | 0.00 | 0.00 | 0.00 | 0.00 | 0.00 | 14.00 |
| FTL1 | 3.66 | 0.00 | 0.00 | 0.00 | 0.00 | 0.00 | 0.00 | 0.00 | 0.00 | 0.00 | 0.00 | 0.00 |
| FURIN | 0.00 | 0.00 | 0.00 | 0.00 | 0.00 | 0.00 | 0.00 | 0.00 | 0.00 | 0.00 | 0.00 | 5.77 |
| FYB | 0.00 | 0.00 | 0.00 | 0.00 | 0.00 | 0.00 | 0.00 | 4.76 | 0.00 | 0.00 | 0.00 | 0.00 |
| GOS2 | 3.50 | 0.00 | 0.00 | 0.00 | 0.00 | 4.63 | 0.00 | 7.54 | 0.00 | 0.00 | 9.84 | 30.39 |
| GADD45A | 0.00 | 0.00 | 0.00 | 0.00 | 0.00 | 0.00 | 0.00 | 0.00 | 0.00 | 0.00 | 0.00 | 3.32 |
| GADD45B | 0.00 | 0.00 | 0.00 | 0.00 | 0.00 | 0.00 | 0.00 | 0.00 | 0.00 | 0.00 | 0.00 | 4.95 |
| GADD45G | 5.95 | 0.00 | 0.00 | 0.00 | 0.00 | 0.00 | 0.00 | 0.00 | 0.00 | 0.00 | 0.00 | 0.00 |
| GAPDH | 0.00 | 0.00 | 0.00 | 0.00 | 0.00 | 0.00 | 0.00 | 0.00 | 0.00 | 0.00 | 0.00 | 0.00 |
| GAS2 | 0.00 | 0.00 | 0.00 | 0.00 | 0.00 | 0.00 | 0.00 | 0.00 | 0.00 | 0.00 | 0.00 | 0.00 |
| GBE1 | 0.00 | 0.00 | 0.00 | 0.00 | 0.00 | 0.00 | 0.00 | 0.00 | 0.00 | 0.00 | 0.00 | 0.00 |
| GBP2 | 0.00 | 0.00 | 0.00 | 0.00 | 0.00 | 0.00 | 3.16 | 0.00 | 0.00 | 0.00 | 0.00 | 0.00 |
| GBP3 | 0.00 | 3.54 | 0.00 | 0.00 | 0.00 | 0.00 | 3.60 | 0.00 | 0.00 | 0.00 | 0.00 | 0.00 |
| GBP4 | 0.00 | 4.25 | 0.00 | 0.00 | 0.00 | 0.00 | 3.18 | 0.00 | 3.62 | 0.00 | 0.00 | 0.00 |
| GBP5 | 0.00 | 0.00 | 0.00 | 0.00 | 0.00 | 0.00 | 6.49 | 0.00 | 0.00 | 0.00 | 0.00 | 0.00 |
| GBP7 | 0.00 | 3.69 | 0.00 | 0.00 | 0.00 | 0.00 | 3.78 | 0.00 | 3.83 | 0.00 | 0.00 | 0.00 |
| GBP8 | 0.00 | 0.00 | 0.00 | 0.00 | 0.00 | 0.00 | 0.00 | 0.00 | 3.69 | 0.00 | 0.00 | 0.00 |
| GBP9 | 0.00 | 0.00 | 0.00 | 0.00 | 0.00 | 0.00 | 0.00 | 0.00 | 3.54 | 0.00 | 0.00 | 0.00 |
| GCNT2 | 0.00 | 0.00 | 0.00 | 0.00 | 0.00 | 0.00 | 0.00 | 0.00 | 3.35 | 0.00 | 0.00 | 0.00 |
| GDPGP1 | 0.00 | 0.00 | 0.00 | 0.00 | 0.00 | 0.00 | 0.00 | 0.00 | 0.00 | 0.00 | 0.00 | 0.00 |
| GEM | 3.64 | 0.00 | 0.00 | 0.00 | 0.00 | 0.00 | 0.00 | 0.00 | 0.00 | 0.00 | 0.00 | 0.00 |
| GIMAP3 | 0.00 | 0.00 | 0.00 | 0.00 | 0.00 | 0.00 | 0.00 | 0.00 | 0.00 | 0.00 | 0.00 | 0.00 |
| GIMAP4 | 0.00 | 0.00 | 0.00 | 0.00 | 0.00 | 0.00 | 0.00 | 0.00 | 0.00 | 0.00 | 0.00 | 0.00 |
| GIMAP5 | 0.00 | 0.00 | 0.00 | 0.00 | 0.00 | 0.00 | 4.18 | 0.00 | 0.00 | 0.00 | 0.00 | 0.00 |
| GIMAP8 | 0.00 | 3.52 | 0.00 | 0.00 | 0.00 | 0.00 | 0.00 | 0.00 | 0.00 | 0.00 | 0.00 | 0.00 |
| GLA | 0.00 | 0.00 | 0.00 | 0.00 | 0.00 | 0.00 | 0.00 | 0.00 | 0.00 | 0.00 | 4.00 | 0.00 |
| GM12185 | 0.00 | 0.00 | 0.00 | 0.00 | 0.00 | 0.00 | 2.98 | 0.00 | 0.00 | 0.00 | 0.00 | 0.00 |
| GM12216 | 0.00 | 0.00 | 0.00 | 10.46 | 0.00 | 4.71 | 0.00 | 0.00 | 0.00 | 0.00 | 0.00 | 0.00 |
| GM12840 | 0.00 | 0.00 | 0.00 | 0.00 | 0.00 | 0.00 | 0.00 | 0.00 | 0.00 | 0.00 | 0.00 | 0.00 |
| GM15987 | 0.00 | 0.00 | 0.00 | 0.00 | 0.00 | 0.00 | 0.00 | 3.58 | 0.00 | 0.00 | 0.00 | 0.00 |
| GM2000 | 5.87 | 0.00 | 0.00 | 0.00 | 0.00 | 0.00 | 0.00 | 0.00 | 0.00 | 0.00 | 0.00 | 0.00 |
| GM26545 | 0.00 | 0.00 | 0.00 | 0.00 | 0.00 | 0.00 | 0.00 | 0.00 | 3.35 | 0.00 | 0.00 | 0.00 |
| GM26699 | 0.00 | 0.00 | 0.00 | 0.00 | 0.00 | 0.00 | 3.00 | 0.00 | 0.00 | 0.00 | 0.00 | 0.00 |
| GM26917 | 0.00 | 0.00 | 0.00 | 6.53 | 0.00 | 0.00 | 0.00 | 0.00 | 0.00 | 0.00 | 0.00 | 0.00 |
| GM37065 | 0.00 | 0.00 | 0.00 | 0.00 | 0.00 | 0.00 | 0.00 | 0.00 | 0.00 | 0.00 | 0.00 | 0.00 |
| GM43603 | 0.00 | 0.00 | 0.00 | 0.00 | 0.00 | 0.00 | 0.00 | 0.00 | 0.00 | 0.00 | 0.00 | 0.00 |
| GM4951 | 0.00 | 0.00 | 0.00 | 0.00 | 0.00 | 0.00 | 0.00 | 0.00 | 0.00 | 0.00 | 0.00 | 0.00 |
| GM8369 | 4.00 | 0.00 | 0.00 | 0.00 | 0.00 | 0.00 | 0.00 | 0.00 | 0.00 | 0.00 | 0.00 | 0.00 |
| GM8797 | 0.00 | 3.42 | 0.00 | 0.00 | 0.00 | 0.00 | 0.00 | 0.00 | 0.00 | 0.00 | 0.00 | 0.00 |
| GM8953 | 0.00 | 4.11 | 0.00 | 0.00 | 0.00 | 0.00 | 0.00 | 0.00 | 0.00 | 0.00 | 0.00 | 0.00 |
| GNB2L1 | 0.00 | 0.00 | 0.00 | 0.00 | 4.01 | 0.00 | 0.00 | 0.00 | 0.00 | 0.00 | 0.00 | 0.00 |
| GOLGA1 | 0.00 | 0.00 | 0.00 | 0.00 | 0.00 | 0.00 | 0.00 | 0.00 | 0.00 | 0.00 | 0.00 | 0.00 |
| GOLGA3 | 0.00 | 0.00 | 0.00 | 0.00 | 5.22 | 0.00 | 0.00 | 0.00 | 0.00 | 0.00 | 0.00 | 0.00 |
| GPATCH2 | 0.00 | 3.05 | 0.00 | 0.00 | 0.00 | 0.00 | 0.00 | 0.00 | 0.00 | 0.00 | 0.00 | 0.00 |
| GPI1 | 0.00 | 0.00 | 0.00 | 0.00 | 0.00 | 0.00 | 0.00 | 0.00 | 0.00 | 0.00 | 0.00 | 3.27 |
| GPNMB | 0.00 | 3.61 | 0.00 | 0.00 | 0.00 | 0.00 | 0.00 | 0.00 | 0.00 | 0.00 | 0.00 | 0.00 |
| GPR35 | 0.00 | 0.00 | 0.00 | 0.00 | 0.00 | 0.00 | 3.10 | 0.00 | 0.00 | 0.00 | 0.00 | 0.00 |
| GRINA | 0.00 | 0.00 | 0.00 | 0.00 | 0.00 | 0.00 | 0.00 | 0.00 | 0.00 | 0.00 | 0.00 | 5.07 |
| GSR | 0.00 | 0.00 | 0.00 | 0.00 | 0.00 | 0.00 | 0.00 | 0.00 | 4.01 | 0.00 | 0.00 | 0.00 |
| GZMA | 0.00 | 0.00 | 0.00 | 0.00 | 0.00 | 0.00 | 0.00 | 0.00 | 0.00 | 0.00 | 0.00 | 0.00 |
| GZMB | 0.00 | 0.00 | 0.00 | 0.00 | 0.00 | 0.00 | 0.00 | 0.00 | 0.00 | 0.00 | 0.00 | 0.00 |
| GZMC | 0.00 | 0.00 | 0.00 | 0.00 | 5.71 | 0.00 | 0.00 | 0.00 | 0.00 | 0.00 | 0.00 | 0.00 |
| GZMF | 0.00 | 0.00 | 0.00 | 0.00 | 0.00 | 0.00 | 0.00 | 0.00 | 0.00 | 0.00 | 0.00 | 0.00 |
| H2-AA | 0.00 | 5.97 | 0.00 | 0.00 | 6.55 | 0.00 | 0.00 | 3.57 | 8.32 | 0.00 | 6.47 | 5.99 |
| H2-AB1 | 0.00 | 4.55 | 0.00 | 0.00 | 5.01 | 0.00 | 0.00 | 4.14 | 8.61 | 0.00 | 6.50 | 5.12 |
| H2AFY | 0.00 | 0.00 | 0.00 | 0.00 | 0.00 | 0.00 | 3.27 | 0.00 | 0.00 | 0.00 | 0.00 | 0.00 |
| H2AFZ | 0.00 | 0.00 | 0.00 | 0.00 | 0.00 | 0.00 | 0.00 | 0.00 | 3.59 | 0.00 | 0.00 | 0.00 |
| H2-D1 | 0.00 | 4.30 | 0.00 | 0.00 | 0.00 | 0.00 | 0.00 | 0.00 | 4.23 | 0.00 | 0.00 | 0.00 |
| H2-DMA | 0.00 | 0.00 | 0.00 | 0.00 | 0.00 | 0.00 | 0.00 | 0.00 | 3.83 | 0.00 | 0.00 | 0.00 |
| H2-DMB1 | 0.00 | 0.00 | 0.00 | 0.00 | 0.00 | 0.00 | 0.00 | 0.00 | 4.80 | 0.00 | 0.00 | 0.00 |
| H2-DMB2 | 0.00 | 0.00 | 0.00 | 0.00 | 0.00 | 0.00 | 0.00 | 0.00 | 0.00 | 0.00 | 0.00 | 0.00 |
| H2-K1 | 0.00 | 4.43 | 0.00 | 0.00 | 0.00 | 3.93 | 0.00 | 0.00 | 5.47 | 0.00 | 0.00 | 0.00 |
| H2-M3 | 0.00 | 3.06 | 0.00 | 0.00 | 0.00 | 0.00 | 0.00 | 0.00 | 0.00 | 0.00 | 0.00 | 0.00 |
| H2-OB | 0.00 | 0.00 | 0.00 | 0.00 | 0.00 | 0.00 | 0.00 | 0.00 | 0.00 | 0.00 | 0.00 | 0.00 |
| H2-Q10 | 0.00 | 0.00 | 0.00 | 0.00 | 0.00 | 0.00 | 0.00 | 4.33 | 0.00 | 0.00 | 0.00 | 0.00 |
| H2-Q4 | 0.00 | 5.94 | 0.00 | 0.00 | 0.00 | 0.00 | 0.00 | 0.00 | 0.00 | 0.00 | 0.00 | 0.00 |
| H2-Q6 | 0.00 | 4.74 | 0.00 | 0.00 | 0.00 | 0.00 | 0.00 | 0.00 | 0.00 | 0.00 | 0.00 | 0.00 |
| H2-Q7 | 0.00 | 4.61 | 0.00 | 0.00 | 0.00 | 4.98 | 0.00 | 0.00 | 0.00 | 0.00 | 0.00 | 0.00 |
| H2-T22 | 0.00 | 3.08 | 0.00 | 0.00 | 0.00 | 0.00 | 0.00 | 0.00 | 4.87 | 0.00 | 0.00 | 0.00 |
| H2-T23 | 0.00 | 0.00 | 0.00 | 0.00 | 0.00 | 0.00 | 0.00 | 0.00 | 3.79 | 0.00 | 0.00 | 0.00 |
| HADHA | 0.00 | 4.16 | 0.00 | 0.00 | 0.00 | 0.00 | 0.00 | 0.00 | 0.00 | 0.00 | 0.00 | 0.00 |
| HBA-A2 | 4.45 | 0.00 | 0.00 | 0.00 | 0.00 | 0.00 | 0.00 | 3.67 | 0.00 | 5.10 | 0.00 | 4.31 |
| HBB-BT | 0.00 | 0.00 | 0.00 | 0.00 | 0.00 | 0.00 | 0.00 | 0.00 | 0.00 | 0.00 | 0.00 | 0.00 |
| HCAR2 | 0.00 | 0.00 | 0.00 | 0.00 | 0.00 | 0.00 | 0.00 | 0.00 | 0.00 | 0.00 | 0.00 | 12.52 |
| HCK | 0.00 | 0.00 | 0.00 | 0.00 | 0.00 | 0.00 | 3.12 | 5.20 | 0.00 | 0.00 | 0.00 | 0.00 |
| HDC | 0.00 | 0.00 | 0.00 | 0.00 | 0.00 | 6.67 | 0.00 | 0.00 | 0.00 | 0.00 | 0.00 | 16.02 |
| HDLBP | 0.00 | 0.00 | 0.00 | 0.00 | 0.00 | 0.00 | 0.00 | 0.00 | 0.00 | 0.00 | 0.00 | 0.00 |
| HEATR5A | 0.00 | 0.00 | 0.00 | 0.00 | 0.00 | 0.00 | 3.03 | 0.00 | 0.00 | 0.00 | 0.00 | 0.00 |
| HEATR5B | 0.00 | 0.00 | 0.00 | 0.00 | 0.00 | 0.00 | 0.00 | 0.00 | 0.00 | 0.00 | 0.00 | 0.00 |
| HELQ | 0.00 | 0.00 | 0.00 | 0.00 | 0.00 | 0.00 | 0.00 | 0.00 | 0.00 | 0.00 | 0.00 | 0.00 |
| HERC6 | 0.00 | 0.00 | 0.00 | 0.00 | 0.00 | 0.00 | 7.94 | 0.00 | 0.00 | 0.00 | 0.00 | 0.00 |
| HES1 | 0.00 | 0.00 | 0.00 | 0.00 | 3.93 | 0.00 | 0.00 | 0.00 | 0.00 | 0.00 | 6.70 | 0.00 |
| HID1 | 0.00 | 3.46 | 0.00 | 0.00 | 0.00 | 0.00 | 0.00 | 0.00 | 0.00 | 0.00 | 0.00 | 0.00 |
| HILPDA | 0.00 | 3.18 | 0.00 | 0.00 | 0.00 | 0.00 | 0.00 | 4.35 | 0.00 | 0.00 | 0.00 | 23.69 |
| HIPK2 | 0.00 | 0.00 | 0.00 | 0.00 | 0.00 | 0.00 | 3.75 | 0.00 | 0.00 | 0.00 | 0.00 | 0.00 |
| HIST1H1C | 4.34 | 3.95 | 0.00 | 0.00 | 0.00 | 0.00 | 0.00 | 0.00 | 0.00 | 0.00 | 0.00 | 0.00 |
| HIST1H1E | 0.00 | 0.00 | 0.00 | 0.00 | 0.00 | 0.00 | 0.00 | 0.00 | 0.00 | 0.00 | 0.00 | 0.00 |
| HIST1H2AE | 0.00 | 0.00 | 4.53 | 0.00 | 0.00 | 0.00 | 0.00 | 0.00 | 0.00 | 0.00 | 0.00 | 0.00 |
| HIST1H2AG | 0.00 | 0.00 | 0.00 | 0.00 | 0.00 | 0.00 | 0.00 | 0.00 | 3.54 | 0.00 | 0.00 | 0.00 |
| HIST1H2BC | 4.98 | 4.19 | 0.00 | 0.00 | 0.00 | 0.00 | 0.00 | 0.00 | 0.00 | 0.00 | 0.00 | 3.23 |
| HIST1H2BJ | 0.00 | 0.00 | 0.00 | 0.00 | 0.00 | 0.00 | 0.00 | 0.00 | 0.00 | 0.00 | 0.00 | 0.00 |
| HIST1H4I | 0.00 | 0.00 | 0.00 | 0.00 | 0.00 | 0.00 | 0.00 | 0.00 | 0.00 | 0.00 | 0.00 | 3.58 |
| HNRNPU | 0.00 | 0.00 | 0.00 | 0.00 | 0.00 | 0.00 | 0.00 | 0.00 | 0.00 | 0.00 | 0.00 | 0.00 |
| HOXA7 | 0.00 | 0.00 | 0.00 | 0.00 | 0.00 | 0.00 | 0.00 | 0.00 | 0.00 | 0.00 | 0.00 | 0.00 |
| HOXB4 | 0.00 | 0.00 | 0.00 | 0.00 | 0.00 | 0.00 | 3.86 | 0.00 | 0.00 | 0.00 | 0.00 | 0.00 |
| HP | 0.00 | 0.00 | 0.00 | 0.00 | 0.00 | 0.00 | 0.00 | 0.00 | 0.00 | 0.00 | 0.00 | 4.60 |
| HP1BP3 | 0.00 | 0.00 | 0.00 | 0.00 | 3.70 | 0.00 | 0.00 | 0.00 | 0.00 | 0.00 | 0.00 | 0.00 |
| HPGDS | 0.00 | 0.00 | 0.00 | 0.00 | 0.00 | 0.00 | 0.00 | 0.00 | 0.00 | 0.00 | 0.00 | 0.00 |
| HSP90AA1 | 6.60 | 0.00 | 0.00 | 0.00 | 0.00 | 0.00 | 0.00 | 0.00 | 3.17 | 0.00 | 0.00 | 0.00 |
| HSP90AB1 | 4.12 | 0.00 | 0.00 | 0.00 | 0.00 | 0.00 | 0.00 | 0.00 | 0.00 | 0.00 | 0.00 | 0.00 |
| HSPA14 | 0.00 | 0.00 | 0.00 | 0.00 | 0.00 | 0.00 | 0.00 | 0.00 | 0.00 | 0.00 | 0.00 | 0.00 |
| HSPA1A | 57.03 | 0.00 | 0.00 | 8.47 | 0.00 | 0.00 | 0.00 | 0.00 | 0.00 | 0.00 | 0.00 | 0.00 |
| HSPA1B | 9.33 | 0.00 | 0.00 | 0.00 | 0.00 | 0.00 | 0.00 | 0.00 | 0.00 | 0.00 | 0.00 | 0.00 |
| HSPA8 | 4.88 | 0.00 | 0.00 | 0.00 | 0.00 | 0.00 | 0.00 | 0.00 | 0.00 | 0.00 | 0.00 | 0.00 |
| HSPD1 | 6.59 | 0.00 | 0.00 | 0.00 | 0.00 | 0.00 | 0.00 | 0.00 | 0.00 | 0.00 | 0.00 | 0.00 |
| HYAL2 | 0.00 | 0.00 | 0.00 | 0.00 | 0.00 | 0.00 | 0.00 | 0.00 | 0.00 | 0.00 | 0.00 | 0.00 |
| ICAM1 | 0.00 | 0.00 | 0.00 | 0.00 | 0.00 | 0.00 | 3.05 | 0.00 | 0.00 | 0.00 | 0.00 | 0.00 |
| ICOS | 0.00 | 0.00 | 0.00 | 0.00 | 0.00 | 0.00 | 0.00 | 4.50 | 0.00 | 0.00 | 0.00 | 0.00 |
| ID1 | 0.00 | 0.00 | 0.00 | 0.00 | 0.00 | 0.00 | 0.00 | 0.00 | 0.00 | 0.00 | 0.00 | 7.18 |
| ID2 | 0.00 | 0.00 | 0.00 | 0.00 | 0.00 | 0.00 | 0.00 | 0.00 | 0.00 | 0.00 | 0.00 | 3.36 |
| ID3 | 0.00 | 0.00 | 0.00 | 0.00 | 0.00 | 0.00 | 0.00 | 0.00 | 0.00 | 0.00 | 0.00 | 0.00 |
| IER2 | 0.00 | 0.00 | 0.00 | 0.00 | 0.00 | 0.00 | 0.00 | 0.00 | 0.00 | 0.00 | 0.00 | 0.00 |
| IER3 | 0.00 | 0.00 | 0.00 | 0.00 | 0.00 | 0.00 | 0.00 | 0.00 | 0.00 | 0.00 | 0.00 | 20.89 |
| IER5 | 3.55 | 0.00 | 0.00 | 0.00 | 0.00 | 0.00 | 0.00 | 0.00 | 0.00 | 0.00 | 0.00 | 0.00 |
| IFI203 | 0.00 | 4.53 | 0.00 | 0.00 | 0.00 | 0.00 | 3.25 | 0.00 | 0.00 | 0.00 | 0.00 | 0.00 |
| IFI204 | 0.00 | 5.54 | 0.00 | 0.00 | 0.00 | 0.00 | 11.06 | 0.00 | 0.00 | 4.98 | 0.00 | 0.00 |
| IFI205 | 0.00 | 3.88 | 0.00 | 0.00 | 0.00 | 0.00 | 10.21 | 5.86 | 7.68 | 7.06 | 0.00 | 0.00 |
| IFI27L2A | 0.00 | 6.07 | 0.00 | 0.00 | 4.74 | 4.68 | 9.19 | 0.00 | 6.59 | 12.58 | 0.00 | 0.00 |
| IFI35 | 0.00 | 3.52 | 0.00 | 0.00 | 0.00 | 0.00 | 3.99 | 0.00 | 0.00 | 0.00 | 0.00 | 0.00 |
| IFI44 | 0.00 | 0.00 | 0.00 | 0.00 | 0.00 | 0.00 | 3.76 | 0.00 | 0.00 | 0.00 | 0.00 | 0.00 |
| IFI47 | 0.00 | 5.28 | 0.00 | 0.00 | 0.00 | 0.00 | 6.03 | 5.21 | 3.53 | 3.54 | 0.00 | 0.00 |
| IFIT1 | 0.00 | 0.00 | 0.00 | 0.00 | 0.00 | 0.00 | 0.00 | 0.00 | 0.00 | 0.00 | 0.00 | 0.00 |
| IFIT2 | 0.00 | 0.00 | 0.00 | 0.00 | 0.00 | 0.00 | 4.84 | 0.00 | 4.92 | 3.91 | 0.00 | 0.00 |
| IFIT3 | 0.00 | 6.32 | 0.00 | 0.00 | 0.00 | 0.00 | 3.52 | 0.00 | 0.00 | 3.95 | 0.00 | 0.00 |
| IFIT3B | 0.00 | 0.00 | 0.00 | 0.00 | 0.00 | 0.00 | 4.15 | 0.00 | 0.00 | 5.67 | 0.00 | 0.00 |
| IFITM1 | 0.00 | 0.00 | 0.00 | 0.00 | 0.00 | 0.00 | 0.00 | 0.00 | 0.00 | 0.00 | 0.00 | 9.41 |
| IFITM2 | 0.00 | 0.00 | 0.00 | 0.00 | 0.00 | 0.00 | 4.48 | 0.00 | 0.00 | 0.00 | 0.00 | 0.00 |
| IFITM6 | 0.00 | 0.00 | 0.00 | 0.00 | 0.00 | 0.00 | 3.68 | 0.00 | 0.00 | 0.00 | 0.00 | 0.00 |
| IFNG | 0.00 | 0.00 | 0.00 | 0.00 | 0.00 | 0.00 | 0.00 | 0.00 | 0.00 | 0.00 | 0.00 | 0.00 |
| IFNGR1 | 0.00 | 0.00 | 0.00 | 0.00 | 0.00 | 0.00 | 0.00 | 0.00 | 0.00 | 0.00 | 0.00 | 0.00 |
| IFRD1 | 3.53 | 0.00 | 0.00 | 0.00 | 0.00 | 0.00 | 0.00 | 0.00 | 0.00 | 0.00 | 0.00 | 4.92 |
| IFT22 | 0.00 | 0.00 | 0.00 | 0.00 | 0.00 | 0.00 | 3.09 | 0.00 | 0.00 | 0.00 | 0.00 | 0.00 |
| IGF2R | 0.00 | 0.00 | 0.00 | 0.00 | 0.00 | 0.00 | 0.00 | 0.00 | 0.00 | 0.00 | 0.00 | 0.00 |
| IGFBP7 | 0.00 | 0.00 | 0.00 | 0.00 | 0.00 | 0.00 | 0.00 | 0.00 | 0.00 | 0.00 | 0.00 | 3.94 |
| IGSF23 | 0.00 | 0.00 | 0.00 | 0.00 | 0.00 | 0.00 | 0.00 | 0.00 | 0.00 | 0.00 | 0.00 | 0.00 |
| IGTP | 0.00 | 3.51 | 0.00 | 0.00 | 0.00 | 0.00 | 0.00 | 0.00 | 0.00 | 0.00 | 0.00 | 0.00 |
| IIGP1 | 0.00 | 5.78 | 0.00 | 0.00 | 0.00 | 0.00 | 0.00 | 0.00 | 0.00 | 6.19 | 8.22 | 0.00 |
| IKBKE | 0.00 | 0.00 | 0.00 | 0.00 | 0.00 | 0.00 | 0.00 | 0.00 | 0.00 | 4.80 | 0.00 | 0.00 |
| IL10RA | 0.00 | 0.00 | 0.00 | 0.00 | 0.00 | 0.00 | 3.46 | 0.00 | 0.00 | 0.00 | 0.00 | 0.00 |
| IL18BP | 0.00 | 4.42 | 0.00 | 0.00 | 0.00 | 0.00 | 4.08 | 0.00 | 4.70 | 0.00 | 0.00 | 0.00 |
| IL1B | 0.00 | 0.00 | 0.00 | 0.00 | 0.00 | 0.00 | 3.91 | 3.74 | 0.00 | 0.00 | 0.00 | 4.22 |
| IL1R2 | 0.00 | 0.00 | 0.00 | 0.00 | 0.00 | 0.00 | 0.00 | 0.00 | 4.25 | 0.00 | 0.00 | 9.85 |
| IL1RN | 0.00 | 0.00 | 0.00 | 0.00 | 0.00 | 0.00 | 0.00 | 0.00 | 0.00 | 0.00 | 0.00 | 13.91 |
| IL3RA | 0.00 | 3.19 | 0.00 | 0.00 | 0.00 | 0.00 | 0.00 | 0.00 | 0.00 | 0.00 | 0.00 | 0.00 |
| IL4I1 | 0.00 | 0.00 | 0.00 | 0.00 | 0.00 | 0.00 | 3.67 | 0.00 | 0.00 | 0.00 | 0.00 | 0.00 |
| INO80 | 0.00 | 0.00 | 0.00 | 0.00 | 0.00 | 0.00 | 0.00 | 0.00 | 0.00 | 0.00 | 0.00 | 0.00 |
| INSIG1 | 0.00 | 0.00 | 0.00 | 0.00 | 0.00 | 4.51 | 0.00 | 0.00 | 0.00 | 0.00 | 0.00 | 0.00 |
| IPCEF1 | 0.00 | 0.00 | 0.00 | 0.00 | 0.00 | 0.00 | 0.00 | 0.00 | 0.00 | 0.00 | 0.00 | 0.00 |
| IQGAP1 | 0.00 | 0.00 | 0.00 | 0.00 | 0.00 | 0.00 | 3.54 | 0.00 | 0.00 | 0.00 | 0.00 | 0.00 |
| IQGAP2 | 0.00 | 0.00 | 0.00 | 0.00 | 0.00 | 0.00 | 0.00 | 0.00 | 0.00 | 0.00 | 0.00 | 0.00 |
| IRAK2 | 0.00 | 0.00 | 0.00 | 0.00 | 0.00 | 0.00 | 3.03 | 0.00 | 0.00 | 0.00 | 0.00 | 0.00 |
| IRF1 | 0.00 | 5.41 | 0.00 | 0.00 | 0.00 | 0.00 | 5.03 | 0.00 | 0.00 | 0.00 | 0.00 | 0.00 |
| IRF7 | 0.00 | 0.00 | 5.92 | 0.00 | 0.00 | 0.00 | 7.93 | 0.00 | 4.62 | 4.02 | 5.41 | 0.00 |
| IRF8 | 0.00 | 3.23 | 0.00 | 0.00 | 0.00 | 0.00 | 0.00 | 0.00 | 0.00 | 5.35 | 0.00 | 0.00 |
| IRF9 | 0.00 | 0.00 | 0.00 | 0.00 | 0.00 | 0.00 | 3.67 | 0.00 | 0.00 | 0.00 | 0.00 | 0.00 |
| IRG1 | 0.00 | 0.00 | 0.00 | 0.00 | 0.00 | 0.00 | 0.00 | 0.00 | 0.00 | 0.00 | 0.00 | 12.11 |
| IRGM1 | 0.00 | 0.00 | 0.00 | 0.00 | 0.00 | 0.00 | 3.60 | 0.00 | 0.00 | 3.96 | 0.00 | 0.00 |
| IRGM2 | 0.00 | 0.00 | 0.00 | 0.00 | 0.00 | 0.00 | 3.59 | 0.00 | 0.00 | 3.66 | 0.00 | 0.00 |
| ISG20 | 0.00 | 0.00 | 0.00 | 0.00 | 0.00 | 0.00 | 3.93 | 0.00 | 0.00 | 0.00 | 0.00 | 0.00 |
| ITGA4 | 0.00 | 0.00 | 0.00 | 0.00 | 0.00 | 0.00 | 4.60 | 0.00 | 0.00 | 0.00 | 0.00 | 0.00 |
| ITGB1 | 0.00 | 0.00 | 0.00 | 0.00 | 0.00 | 0.00 | 0.00 | 0.00 | 3.71 | 0.00 | 0.00 | 0.00 |
| ITIH5 | 0.00 | 0.00 | 0.00 | 0.00 | 0.00 | 0.00 | 0.00 | 0.00 | 0.00 | 0.00 | 0.00 | 0.00 |
| ITM2A | 0.00 | 0.00 | 0.00 | 0.00 | 0.00 | 0.00 | 0.00 | 0.00 | 0.00 | 5.35 | 0.00 | 0.00 |
| ITM2B | 0.00 | 0.00 | 0.00 | 0.00 | 0.00 | 0.00 | 0.00 | 0.00 | 3.26 | 0.00 | 0.00 | 0.00 |
| ITSN1 | 0.00 | 0.00 | 0.00 | 0.00 | 0.00 | 0.00 | 0.00 | 0.00 | 0.00 | 0.00 | 0.00 | 3.43 |
| JAKMIP1 | 0.00 | 0.00 | 0.00 | 0.00 | 0.00 | 4.85 | 0.00 | 0.00 | 0.00 | 0.00 | 0.00 | 0.00 |
| JUN | 6.78 | 0.00 | 0.00 | 0.00 | 0.00 | 0.00 | 0.00 | 0.00 | 0.00 | 0.00 | 0.00 | 0.00 |
| JUNB | 9.85 | 0.00 | 0.00 | 0.00 | 0.00 | 0.00 | 0.00 | 0.00 | 0.00 | 0.00 | 0.00 | 5.64 |
| KDM6B | 3.74 | 0.00 | 0.00 | 0.00 | 0.00 | 0.00 | 0.00 | 0.00 | 0.00 | 0.00 | 0.00 | 0.00 |
| KIF20B | 0.00 | 0.00 | 0.00 | 0.00 | 0.00 | 0.00 | 0.00 | 0.00 | 0.00 | 0.00 | 0.00 | 0.00 |
| KIF3B | 0.00 | 0.00 | 0.00 | 0.00 | 0.00 | 0.00 | 3.97 | 0.00 | 0.00 | 0.00 | 0.00 | 0.00 |
| KIFC1 | 0.00 | 0.00 | 0.00 | 0.00 | 0.00 | 0.00 | 0.00 | 0.00 | 0.00 | 0.00 | 0.00 | 0.00 |
| KLC4 | 0.00 | 0.00 | 0.00 | 0.00 | 0.00 | 0.00 | 3.22 | 0.00 | 0.00 | 0.00 | 0.00 | 0.00 |
| KLF2 | 0.00 | 0.00 | 0.00 | 0.00 | 0.00 | 0.00 | 0.00 | 0.00 | 0.00 | 0.00 | 0.00 | 0.00 |
| KLF6 | 0.00 | 3.81 | 0.00 | 0.00 | 0.00 | 0.00 | 0.00 | 0.00 | 0.00 | 0.00 | 0.00 | 0.00 |
| KLF9 | 0.00 | 3.22 | 0.00 | 0.00 | 0.00 | 0.00 | 0.00 | 0.00 | 0.00 | 0.00 | 0.00 | 0.00 |
| KLK8 | 0.00 | 0.00 | 0.00 | 0.00 | 0.00 | 0.00 | 3.06 | 0.00 | 0.00 | 0.00 | 0.00 | 0.00 |
| KLRA4 | 0.00 | 0.00 | 0.00 | 0.00 | 0.00 | 0.00 | 0.00 | 0.00 | 4.16 | 0.00 | 0.00 | 0.00 |
| KLRA8 | 0.00 | 0.00 | 0.00 | 0.00 | 0.00 | 0.00 | 0.00 | 0.00 | 3.60 | 0.00 | 0.00 | 0.00 |
| KLRA9 | 0.00 | 0.00 | 0.00 | 0.00 | 0.00 | 0.00 | 0.00 | 0.00 | 0.00 | 0.00 | 0.00 | 0.00 |
| KLRC1 | 0.00 | 0.00 | 0.00 | 0.00 | 0.00 | 0.00 | 0.00 | 0.00 | 0.00 | 0.00 | 0.00 | 0.00 |
| KLRD1 | 0.00 | 0.00 | 0.00 | 0.00 | 0.00 | 0.00 | 0.00 | 0.00 | 0.00 | 0.00 | 0.00 | 0.00 |
| KLRK1 | 0.00 | 0.00 | 0.00 | 0.00 | 0.00 | 0.00 | 4.41 | 0.00 | 5.44 | 0.00 | 0.00 | 0.00 |
| KMO | 0.00 | 3.38 | 0.00 | 0.00 | 0.00 | 0.00 | 0.00 | 0.00 | 0.00 | 0.00 | 0.00 | 0.00 |
| KMT2E | 0.00 | 0.00 | 0.00 | 0.00 | 0.00 | 0.00 | 0.00 | 0.00 | 0.00 | 0.00 | 0.00 | 0.00 |
| LACC1 | 0.00 | 5.87 | 0.00 | 0.00 | 0.00 | 0.00 | 0.00 | 0.00 | 5.64 | 0.00 | 0.00 | 0.00 |
| LAPTM5 | 0.00 | 3.79 | 0.00 | 0.00 | 0.00 | 0.00 | 0.00 | 0.00 | 0.00 | 0.00 | 0.00 | 0.00 |
| LCK | 0.00 | 0.00 | 0.00 | 0.00 | 0.00 | 0.00 | 3.35 | 0.00 | 0.00 | 0.00 | 0.00 | 0.00 |
| LCP1 | 0.00 | 0.00 | 0.00 | 0.00 | 0.00 | 0.00 | 0.00 | 0.00 | 0.00 | 0.00 | 0.00 | 0.00 |
| LDHA | 0.00 | 0.00 | 0.00 | 0.00 | 0.00 | 0.00 | 0.00 | 0.00 | 0.00 | 0.00 | 0.00 | 5.86 |
| LEPR | 0.00 | 0.00 | 0.00 | 5.24 | 0.00 | 0.00 | 0.00 | 0.00 | 0.00 | 0.00 | 0.00 | 0.00 |
| LGALS3 | 0.00 | 0.00 | 0.00 | 0.00 | 0.00 | 0.00 | 0.00 | 0.00 | 0.00 | 0.00 | 0.00 | 3.31 |
| LGALS3BP | 0.00 | 3.39 | 0.00 | 0.00 | 0.00 | 0.00 | 0.00 | 0.00 | 3.43 | 0.00 | 0.00 | 0.00 |
| LGALS9 | 0.00 | 0.00 | 0.00 | 0.00 | 0.00 | 0.00 | 0.00 | 0.00 | 3.40 | 0.00 | 0.00 | 0.00 |
| LILR4B | 0.00 | 0.00 | 0.00 | 0.00 | 0.00 | 0.00 | 0.00 | 0.00 | 0.00 | 0.00 | 0.00 | 7.03 |
| LILRB4A | 0.00 | 0.00 | 0.00 | 0.00 | 0.00 | 0.00 | 0.00 | 0.00 | 0.00 | 0.00 | 0.00 | 4.11 |
| LITAF | 0.00 | 0.00 | 0.00 | 0.00 | 0.00 | 0.00 | 0.00 | 0.00 | 0.00 | 0.00 | 0.00 | 4.79 |
| LMBRD2 | 0.00 | 0.00 | 0.00 | 0.00 | 0.00 | 0.00 | 0.00 | 0.00 | 0.00 | 0.00 | 0.00 | 0.00 |
| LMNB1 | 0.00 | 0.00 | 0.00 | 0.00 | 0.00 | 0.00 | 0.00 | 0.00 | 0.00 | 0.00 | 0.00 | 5.79 |
| LMO4 | 0.00 | 0.00 | 0.00 | 0.00 | 0.00 | 0.00 | 0.00 | 0.00 | 0.00 | 0.00 | 0.00 | 0.00 |
| LRBA | 0.00 | 0.00 | 0.00 | 0.00 | 0.00 | 0.00 | 0.00 | 0.00 | 0.00 | 0.00 | 0.00 | 0.00 |
| LRG1 | 0.00 | 0.00 | 0.00 | 0.00 | 0.00 | 7.25 | 0.00 | 6.01 | 0.00 | 0.00 | 6.77 | 19.84 |
| LRRC25 | 0.00 | 0.00 | 0.00 | 0.00 | 0.00 | 0.00 | 0.00 | 0.00 | 4.54 | 0.00 | 0.00 | 0.00 |
| LRRK1 | 0.00 | 0.00 | 0.00 | 0.00 | 0.00 | 0.00 | 5.21 | 0.00 | 0.00 | 0.00 | 0.00 | 0.00 |
| LSM12 | 0.00 | 0.00 | 0.00 | 0.00 | 4.37 | 0.00 | 0.00 | 0.00 | 0.00 | 0.00 | 0.00 | 0.00 |
| LSP1 | 0.00 | 0.00 | 0.00 | 0.00 | 0.00 | 0.00 | 4.47 | 0.00 | 0.00 | 0.00 | 0.00 | 0.00 |
| LTA | 0.00 | 0.00 | 0.00 | 0.00 | 0.00 | 3.83 | 0.00 | 0.00 | 0.00 | 0.00 | 0.00 | 0.00 |
| LY6A | 0.00 | 6.78 | 0.00 | 0.00 | 4.29 | 5.84 | 5.76 | 0.00 | 9.60 | 3.76 | 0.00 | 0.00 |
| LY6C2 | 0.00 | 8.23 | 5.32 | 0.00 | 0.00 | 8.13 | 3.68 | 0.00 | 8.88 | 0.00 | 4.31 | 4.24 |
| LY6D | 0.00 | 0.00 | 9.34 | 0.00 | 0.00 | 0.00 | 0.00 | 0.00 | 0.00 | 0.00 | 0.00 | 0.00 |
| LY6E | 0.00 | 7.62 | 0.00 | 0.00 | 4.96 | 0.00 | 3.61 | 4.23 | 5.84 | 0.00 | 0.00 | 0.00 |
| LY6I | 0.00 | 0.00 | 0.00 | 0.00 | 0.00 | 0.00 | 0.00 | 0.00 | 0.00 | 4.09 | 0.00 | 0.00 |
| LY86 | 0.00 | 3.77 | 0.00 | 0.00 | 0.00 | 0.00 | 3.34 | 0.00 | 0.00 | 0.00 | 0.00 | 0.00 |
| LYSMD3 | 0.00 | 0.00 | 0.00 | 0.00 | 0.00 | 0.00 | 0.00 | 0.00 | 0.00 | 0.00 | 0.00 | 0.00 |
| LYZ1 | 0.00 | 0.00 | 0.00 | 0.00 | 0.00 | 0.00 | 0.00 | 0.00 | 4.69 | 0.00 | 0.00 | 0.00 |
| MACROD1 | 0.00 | 0.00 | 0.00 | 0.00 | 0.00 | 0.00 | 0.00 | 0.00 | 0.00 | 0.00 | 0.00 | 0.00 |
| MAF | 0.00 | 3.39 | 0.00 | 0.00 | 0.00 | 0.00 | 0.00 | 0.00 | 0.00 | 0.00 | 0.00 | 0.00 |
| MAFF | 0.00 | 0.00 | 0.00 | 0.00 | 0.00 | 0.00 | 0.00 | 0.00 | 0.00 | 0.00 | 0.00 | 3.35 |
| MAP3K8 | 6.86 | 0.00 | 0.00 | 0.00 | 0.00 | 0.00 | 0.00 | 0.00 | 0.00 | 0.00 | 0.00 | 0.00 |
| MARCH5 | 0.00 | 0.00 | 0.00 | 0.00 | 0.00 | 0.00 | 4.41 | 0.00 | 0.00 | 0.00 | 0.00 | 0.00 |
| MARCKS | 0.00 | 0.00 | 0.00 | 5.05 | 0.00 | 0.00 | 0.00 | 0.00 | 0.00 | 0.00 | 0.00 | 0.00 |
| MARCKSL1 | 0.00 | 0.00 | 0.00 | 0.00 | 0.00 | 0.00 | 0.00 | 0.00 | 0.00 | 3.78 | 0.00 | 0.00 |
| MASTL | 0.00 | 0.00 | 0.00 | 0.00 | 0.00 | 4.66 | 0.00 | 0.00 | 0.00 | 0.00 | 0.00 | 0.00 |
| MBNL2 | 0.00 | 0.00 | 0.00 | 0.00 | 0.00 | 0.00 | 0.00 | 0.00 | 0.00 | 0.00 | 0.00 | 3.28 |
| MEF2C | 0.00 | 0.00 | 0.00 | 0.00 | 0.00 | 0.00 | 0.00 | 0.00 | 0.00 | 0.00 | 0.00 | 0.00 |
| MEFV | 0.00 | 4.35 | 0.00 | 0.00 | 0.00 | 0.00 | 0.00 | 0.00 | 0.00 | 0.00 | 0.00 | 0.00 |
| METRNL | 0.00 | 0.00 | 0.00 | 0.00 | 0.00 | 0.00 | 0.00 | 3.76 | 0.00 | 0.00 | 0.00 | 0.00 |
| MIF | 0.00 | 0.00 | 0.00 | 0.00 | 0.00 | 0.00 | 0.00 | 0.00 | 0.00 | 0.00 | 0.00 | 8.44 |
| MLANA | 0.00 | 3.72 | 0.00 | 0.00 | 3.75 | 0.00 | 3.72 | 0.00 | 0.00 | 0.00 | 0.00 | 0.00 |
| MLKL | 0.00 | 3.54 | 0.00 | 0.00 | 0.00 | 0.00 | 0.00 | 0.00 | 5.63 | 0.00 | 0.00 | 0.00 |
| MLLT3 | 0.00 | 0.00 | 0.00 | 0.00 | 0.00 | 0.00 | 0.00 | 0.00 | 0.00 | 0.00 | 0.00 | 3.43 |
| MMP12 | 0.00 | 0.00 | 0.00 | 0.00 | 0.00 | 0.00 | 4.85 | 0.00 | 0.00 | 0.00 | 0.00 | 0.00 |
| MMP8 | 0.00 | 3.24 | 0.00 | 0.00 | 0.00 | 0.00 | 0.00 | 4.80 | 4.40 | 0.00 | 0.00 | 12.85 |
| MMP9 | 0.00 | 4.13 | 0.00 | 0.00 | 0.00 | 4.76 | 0.00 | 0.00 | 0.00 | 0.00 | 0.00 | 8.84 |
| MNDA | 0.00 | 5.73 | 0.00 | 0.00 | 0.00 | 0.00 | 10.29 | 4.58 | 0.00 | 5.29 | 0.00 | 0.00 |
| MNDAL | 0.00 | 5.61 | 0.00 | 0.00 | 0.00 | 0.00 | 0.00 | 0.00 | 4.89 | 0.00 | 0.00 | 0.00 |
| MOB1A | 0.00 | 0.00 | 0.00 | 0.00 | 0.00 | 0.00 | 0.00 | 0.00 | 0.00 | 0.00 | 0.00 | 0.00 |
| MOB3C | 0.00 | 3.07 | 0.00 | 0.00 | 0.00 | 0.00 | 0.00 | 0.00 | 0.00 | 0.00 | 0.00 | 0.00 |
| MPEG1 | 0.00 | 4.02 | 0.00 | 0.00 | 0.00 | 0.00 | 5.65 | 0.00 | 0.00 | 0.00 | 0.00 | 0.00 |
| MPP1 | 0.00 | 0.00 | 0.00 | 0.00 | 0.00 | 0.00 | 0.00 | 0.00 | 4.33 | 0.00 | 0.00 | 0.00 |
| MRC1 | 0.00 | 0.00 | 0.00 | 0.00 | 0.00 | 0.00 | 0.00 | 0.00 | 3.33 | 0.00 | 0.00 | 0.00 |
| MRPL1 | 0.00 | 0.00 | 0.00 | 0.00 | 0.00 | 0.00 | 3.66 | 0.00 | 0.00 | 0.00 | 0.00 | 0.00 |
| MS4A1 | 0.00 | 0.00 | 0.00 | 0.00 | 0.00 | 0.00 | 0.00 | 0.00 | 0.00 | 0.00 | 0.00 | 0.00 |
| MS4A4B | 0.00 | 0.00 | 0.00 | 0.00 | 0.00 | 4.19 | 0.00 | 0.00 | 0.00 | 0.00 | 0.00 | 0.00 |
| MS4A4C | 0.00 | 3.07 | 0.00 | 0.00 | 0.00 | 0.00 | 8.46 | 0.00 | 3.47 | 4.07 | 0.00 | 0.00 |
| MS4A6B | 0.00 | 4.20 | 0.00 | 0.00 | 0.00 | 0.00 | 6.41 | 0.00 | 3.51 | 0.00 | 0.00 | 0.00 |
| MS4A6C | 0.00 | 4.69 | 0.00 | 0.00 | 0.00 | 4.47 | 6.42 | 0.00 | 4.05 | 0.00 | 0.00 | 0.00 |
| MS4A7 | 0.00 | 0.00 | 0.00 | 0.00 | 0.00 | 0.00 | 0.00 | 0.00 | 0.00 | 0.00 | 0.00 | 0.00 |
| MT1 | 0.00 | 0.00 | 0.00 | 0.00 | 0.00 | 0.00 | 0.00 | 0.00 | 0.00 | 0.00 | 0.00 | 0.00 |
| MT2 | 0.00 | 0.00 | 0.00 | 0.00 | 0.00 | 0.00 | 0.00 | 0.00 | 0.00 | 0.00 | 0.00 | 0.00 |
| MVP | 0.00 | 0.00 | 0.00 | 0.00 | 0.00 | 0.00 | 3.98 | 0.00 | 0.00 | 0.00 | 0.00 | 0.00 |
| MX1 | 0.00 | 3.14 | 0.00 | 0.00 | 0.00 | 0.00 | 3.96 | 0.00 | 0.00 | 0.00 | 0.00 | 0.00 |
| MXD1 | 0.00 | 0.00 | 0.00 | 0.00 | 0.00 | 0.00 | 0.00 | 0.00 | 0.00 | 0.00 | 0.00 | 7.83 |
| MYC | 6.26 | 0.00 | 0.00 | 0.00 | 0.00 | 0.00 | 0.00 | 0.00 | 0.00 | 0.00 | 0.00 | 0.00 |
| MYL4 | 0.00 | 0.00 | 0.00 | 0.00 | 0.00 | 0.00 | 0.00 | 0.00 | 0.00 | 5.79 | 0.00 | 0.00 |
| MYO5A | 0.00 | 3.63 | 0.00 | 0.00 | 0.00 | 0.00 | 0.00 | 0.00 | 0.00 | 0.00 | 0.00 | 0.00 |
| MZB1 | 0.00 | 0.00 | 0.00 | 0.00 | 0.00 | 0.00 | 0.00 | 0.00 | 0.00 | 0.00 | 0.00 | 0.00 |
| NAAA | 0.00 | 0.00 | 0.00 | 0.00 | 0.00 | 0.00 | 0.00 | 0.00 | 4.12 | 0.00 | 3.97 | 0.00 |
| NABP1 | 0.00 | 0.00 | 0.00 | 0.00 | 5.76 | 0.00 | 0.00 | 0.00 | 0.00 | 0.00 | 0.00 | 0.00 |
| NAPSA | 0.00 | 0.00 | 0.00 | 0.00 | 0.00 | 0.00 | 3.23 | 0.00 | 0.00 | 0.00 | 0.00 | 0.00 |
| NCEH1 | 0.00 | 0.00 | 0.00 | 0.00 | 4.04 | 0.00 | 0.00 | 0.00 | 0.00 | 0.00 | 0.00 | 0.00 |
| NCOA6 | 0.00 | 0.00 | 0.00 | 0.00 | 0.00 | 0.00 | 0.00 | 0.00 | 0.00 | 0.00 | 0.00 | 0.00 |
| NCOA7 | 0.00 | 0.00 | 0.00 | 0.00 | 3.96 | 0.00 | 0.00 | 0.00 | 0.00 | 0.00 | 0.00 | 0.00 |
| NCR1 | 0.00 | 0.00 | 0.00 | 0.00 | 0.00 | 0.00 | 0.00 | 0.00 | 0.00 | 0.00 | 0.00 | 0.00 |
| NEAT1 | 0.00 | 0.00 | 0.00 | 0.00 | 4.19 | 6.20 | 0.00 | 0.00 | 0.00 | 3.57 | 0.00 | 0.00 |
| NECAP1 | 0.00 | 4.95 | 0.00 | 0.00 | 0.00 | 0.00 | 0.00 | 0.00 | 0.00 | 0.00 | 0.00 | 0.00 |
| NEDD9 | 0.00 | 0.00 | 0.00 | 0.00 | 0.00 | 0.00 | 0.00 | 0.00 | 0.00 | 0.00 | 0.00 | 0.00 |
| NFKBIA | 4.06 | 0.00 | 0.00 | 0.00 | 0.00 | 0.00 | 0.00 | 0.00 | 0.00 | 0.00 | 0.00 | 5.76 |
| NFKBIE | 0.00 | 0.00 | 0.00 | 0.00 | 0.00 | 0.00 | 3.52 | 0.00 | 0.00 | 0.00 | 0.00 | 0.00 |
| NFKBIZ | 0.00 | 0.00 | 0.00 | 0.00 | 0.00 | 0.00 | 0.00 | 0.00 | 0.00 | 0.00 | 0.00 | 0.00 |
| NHSL2 | 0.00 | 4.91 | 0.00 | 0.00 | 0.00 | 0.00 | 0.00 | 0.00 | 0.00 | 0.00 | 0.00 | 0.00 |
| NINJ1 | 0.00 | 0.00 | 0.00 | 0.00 | 0.00 | 0.00 | 0.00 | 0.00 | 0.00 | 0.00 | 0.00 | 6.01 |
| NKG7 | 0.00 | 0.00 | 0.00 | 0.00 | 0.00 | 0.00 | 0.00 | 0.00 | 0.00 | 0.00 | 0.00 | 0.00 |
| NLRC5 | 0.00 | 4.00 | 0.00 | 0.00 | 0.00 | 0.00 | 3.24 | 0.00 | 0.00 | 0.00 | 0.00 | 0.00 |
| NMI | 0.00 | 3.46 | 0.00 | 0.00 | 0.00 | 0.00 | 0.00 | 0.00 | 3.33 | 0.00 | 0.00 | 0.00 |
| NOC4L | 0.00 | 0.00 | 0.00 | 0.00 | 0.00 | 0.00 | 3.01 | 0.00 | 0.00 | 0.00 | 0.00 | 0.00 |
| NPC2 | 0.00 | 0.00 | 0.00 | 0.00 | 0.00 | 0.00 | 0.00 | 0.00 | 0.00 | 5.18 | 0.00 | 0.00 |
| NR4A2 | 0.00 | 0.00 | 0.00 | 0.00 | 0.00 | 0.00 | 0.00 | 0.00 | 0.00 | 0.00 | 0.00 | 0.00 |
| NRBF2 | 3.49 | 0.00 | 0.00 | 0.00 | 0.00 | 0.00 | 0.00 | 0.00 | 0.00 | 0.00 | 0.00 | 0.00 |
| NRP1 | 0.00 | 0.00 | 0.00 | 0.00 | 0.00 | 0.00 | 3.02 | 0.00 | 0.00 | 0.00 | 0.00 | 0.00 |
| OAS1A | 0.00 | 3.29 | 0.00 | 0.00 | 0.00 | 0.00 | 5.34 | 0.00 | 0.00 | 0.00 | 0.00 | 0.00 |
| OAS1G | 0.00 | 0.00 | 0.00 | 0.00 | 0.00 | 0.00 | 3.27 | 0.00 | 0.00 | 0.00 | 0.00 | 0.00 |
| OAS2 | 0.00 | 3.03 | 0.00 | 0.00 | 0.00 | 0.00 | 0.00 | 0.00 | 0.00 | 0.00 | 0.00 | 0.00 |
| OASL1 | 0.00 | 0.00 | 0.00 | 0.00 | 0.00 | 0.00 | 4.59 | 0.00 | 0.00 | 0.00 | 0.00 | 0.00 |
| OASL2 | 0.00 | 7.09 | 0.00 | 0.00 | 0.00 | 0.00 | 7.51 | 0.00 | 5.04 | 0.00 | 0.00 | 0.00 |
| ODC1 | 0.00 | 0.00 | 0.00 | 0.00 | 0.00 | 0.00 | 0.00 | 0.00 | 0.00 | 0.00 | 0.00 | 0.00 |
| OSM | 0.00 | 0.00 | 0.00 | 0.00 | 0.00 | 0.00 | 0.00 | 0.00 | 0.00 | 0.00 | 0.00 | 23.76 |
| P2RY6 | 0.00 | 0.00 | 0.00 | 0.00 | 0.00 | 0.00 | 0.00 | 5.90 | 0.00 | 0.00 | 0.00 | 0.00 |
| PAK2 | 0.00 | 0.00 | 0.00 | 0.00 | 0.00 | 0.00 | 0.00 | 0.00 | 0.00 | 0.00 | 0.00 | 0.00 |
| PARP11 | 0.00 | 3.96 | 0.00 | 0.00 | 0.00 | 0.00 | 0.00 | 0.00 | 0.00 | 0.00 | 0.00 | 0.00 |
| PARP12 | 0.00 | 3.70 | 0.00 | 0.00 | 0.00 | 0.00 | 0.00 | 0.00 | 0.00 | 0.00 | 0.00 | 0.00 |
| PARP14 | 0.00 | 5.63 | 0.00 | 0.00 | 0.00 | 0.00 | 7.20 | 0.00 | 0.00 | 3.56 | 0.00 | 0.00 |
| PARP3 | 0.00 | 0.00 | 0.00 | 0.00 | 0.00 | 0.00 | 0.00 | 0.00 | 0.00 | 0.00 | 0.00 | 0.00 |
| PARP9 | 0.00 | 0.00 | 0.00 | 0.00 | 0.00 | 0.00 | 0.00 | 0.00 | 0.00 | 0.00 | 0.00 | 0.00 |
| PBK | 0.00 | 0.00 | 0.00 | 0.00 | 0.00 | 0.00 | 0.00 | 0.00 | 0.00 | 0.00 | 0.00 | 0.00 |
| PCNX | 0.00 | 0.00 | 0.00 | 0.00 | 0.00 | 0.00 | 3.06 | 0.00 | 0.00 | 0.00 | 0.00 | 0.00 |
| PDCD4 | 0.00 | 0.00 | 0.00 | 0.00 | 0.00 | 0.00 | 4.56 | 0.00 | 0.00 | 0.00 | 0.00 | 0.00 |
| PDE4B | 0.00 | 0.00 | 0.00 | 0.00 | 0.00 | 0.00 | 0.00 | 0.00 | 4.03 | 0.00 | 0.00 | 0.00 |
| PEX11G | 0.00 | 0.00 | 0.00 | 0.00 | 0.00 | 0.00 | 0.00 | 3.57 | 0.00 | 0.00 | 0.00 | 0.00 |
| PGLYRP1 | 0.00 | 3.19 | 0.00 | 0.00 | 0.00 | 0.00 | 0.00 | 0.00 | 0.00 | 0.00 | 0.00 | 3.34 |
| PGP | 0.00 | 3.37 | 0.00 | 0.00 | 0.00 | 0.00 | 0.00 | 0.00 | 0.00 | 0.00 | 0.00 | 0.00 |
| PGS1 | 0.00 | 0.00 | 0.00 | 0.00 | 0.00 | 0.00 | 0.00 | 0.00 | 0.00 | 3.56 | 0.00 | 0.00 |
| PHF11A | 0.00 | 0.00 | 4.07 | 0.00 | 0.00 | 0.00 | 4.44 | 0.00 | 0.00 | 0.00 | 0.00 | 0.00 |
| PHF11B | 0.00 | 4.80 | 0.00 | 0.00 | 0.00 | 0.00 | 7.15 | 0.00 | 3.51 | 4.14 | 0.00 | 0.00 |
| PHF11D | 0.00 | 6.14 | 0.00 | 0.00 | 0.00 | 0.00 | 6.83 | 0.00 | 5.09 | 4.44 | 0.00 | 0.00 |
| PHLDA1 | 11.15 | 0.00 | 0.00 | 0.00 | 0.00 | 0.00 | 3.65 | 0.00 | 0.00 | 0.00 | 0.00 | 0.00 |
| PIGM | 0.00 | 0.00 | 0.00 | 0.00 | 5.19 | 0.00 | 0.00 | 0.00 | 0.00 | 0.00 | 0.00 | 0.00 |
| PIK3AP1 | 0.00 | 0.00 | 0.00 | 0.00 | 0.00 | 0.00 | 3.81 | 0.00 | 0.00 | 0.00 | 0.00 | 0.00 |
| PIK3IP1 | 0.00 | 0.00 | 5.32 | 0.00 | 0.00 | 0.00 | 0.00 | 0.00 | 0.00 | 3.80 | 0.00 | 0.00 |
| PILRA | 0.00 | 0.00 | 0.00 | 0.00 | 0.00 | 0.00 | 6.11 | 0.00 | 0.00 | 0.00 | 0.00 | 0.00 |
| PIM1 | 0.00 | 0.00 | 0.00 | 0.00 | 0.00 | 0.00 | 0.00 | 0.00 | 0.00 | 0.00 | 0.00 | 3.78 |
| PIP4K2A | 0.00 | 0.00 | 0.00 | 0.00 | 0.00 | 0.00 | 3.93 | 0.00 | 0.00 | 0.00 | 0.00 | 0.00 |
| PIRB | 0.00 | 3.57 | 0.00 | 0.00 | 0.00 | 0.00 | 0.00 | 0.00 | 0.00 | 0.00 | 0.00 | 0.00 |
| PKIB | 0.00 | 0.00 | 0.00 | 0.00 | 0.00 | 0.00 | 0.00 | 0.00 | 0.00 | 0.00 | 0.00 | 0.00 |
| PKM | 0.00 | 0.00 | 0.00 | 0.00 | 0.00 | 0.00 | 0.00 | 0.00 | 0.00 | 0.00 | 0.00 | 0.00 |
| PLAC8 | 0.00 | 0.00 | 0.00 | 0.00 | 0.00 | 0.00 | 5.27 | 0.00 | 0.00 | 0.00 | 0.00 | 0.00 |
| PLAUR | 0.00 | 0.00 | 0.00 | 0.00 | 0.00 | 0.00 | 0.00 | 0.00 | 0.00 | 0.00 | 0.00 | 4.13 |
| PLBD2 | 0.00 | 0.00 | 0.00 | 0.00 | 0.00 | 0.00 | 0.00 | 0.00 | 0.00 | 0.00 | 0.00 | 0.00 |
| PLEK | 0.00 | 0.00 | 0.00 | 0.00 | 3.68 | 0.00 | 4.03 | 0.00 | 0.00 | 0.00 | 0.00 | 0.00 |
| PLEKHO2 | 0.00 | 3.05 | 0.00 | 0.00 | 0.00 | 0.00 | 0.00 | 0.00 | 0.00 | 0.00 | 0.00 | 0.00 |
| PLIN2 | 0.00 | 0.00 | 0.00 | 0.00 | 0.00 | 0.00 | 0.00 | 0.00 | 0.00 | 0.00 | 0.00 | 4.65 |
| PLK1 | 0.00 | 3.04 | 0.00 | 0.00 | 0.00 | 0.00 | 0.00 | 0.00 | 0.00 | 0.00 | 0.00 | 0.00 |
| PLK2 | 0.00 | 0.00 | 0.00 | 0.00 | 0.00 | 0.00 | 0.00 | 0.00 | 0.00 | 0.00 | 0.00 | 0.00 |
| PLK3 | 0.00 | 0.00 | 0.00 | 0.00 | 0.00 | 0.00 | 0.00 | 0.00 | 0.00 | 0.00 | 0.00 | 7.63 |
| PLP2 | 0.00 | 0.00 | 0.00 | 0.00 | 0.00 | 0.00 | 0.00 | 0.00 | 3.30 | 0.00 | 0.00 | 0.00 |
| PLSCR1 | 0.00 | 0.00 | 0.00 | 0.00 | 0.00 | 0.00 | 0.00 | 0.00 | 0.00 | 0.00 | 0.00 | 5.62 |
| PLTP | 0.00 | 3.27 | 0.00 | 0.00 | 0.00 | 0.00 | 0.00 | 5.11 | 0.00 | 0.00 | 0.00 | 0.00 |
| PMAIP1 | 3.82 | 0.00 | 0.00 | 0.00 | 0.00 | 0.00 | 0.00 | 0.00 | 0.00 | 0.00 | 0.00 | 0.00 |
| PML | 0.00 | 0.00 | 0.00 | 0.00 | 0.00 | 0.00 | 3.10 | 0.00 | 3.82 | 0.00 | 0.00 | 0.00 |
| PNP | 0.00 | 4.13 | 0.00 | 0.00 | 0.00 | 0.00 | 5.77 | 0.00 | 3.74 | 0.00 | 0.00 | 0.00 |
| PNRC1 | 0.00 | 0.00 | 0.00 | 0.00 | 0.00 | 0.00 | 0.00 | 0.00 | 0.00 | 0.00 | 0.00 | 4.51 |
| POU2F2 | 0.00 | 0.00 | 0.00 | 0.00 | 0.00 | 0.00 | 3.42 | 0.00 | 0.00 | 0.00 | 0.00 | 0.00 |
| PPP1R10 | 0.00 | 0.00 | 0.00 | 0.00 | 0.00 | 4.55 | 0.00 | 0.00 | 0.00 | 0.00 | 0.00 | 0.00 |
| PPP1R15A | 13.11 | 0.00 | 4.92 | 0.00 | 0.00 | 0.00 | 0.00 | 0.00 | 0.00 | 0.00 | 0.00 | 3.86 |
| PPP1R15B | 0.00 | 0.00 | 0.00 | 0.00 | 0.00 | 0.00 | 3.09 | 0.00 | 0.00 | 0.00 | 0.00 | 0.00 |
| PPP1R16B | 5.76 | 0.00 | 0.00 | 0.00 | 0.00 | 0.00 | 0.00 | 0.00 | 0.00 | 0.00 | 0.00 | 0.00 |
| PPP2R3C | 0.00 | 0.00 | 0.00 | 0.00 | 0.00 | 0.00 | 0.00 | 0.00 | 0.00 | 0.00 | 0.00 | 3.51 |
| PPP6R3 | 4.13 | 0.00 | 0.00 | 0.00 | 0.00 | 0.00 | 0.00 | 0.00 | 0.00 | 0.00 | 0.00 | 0.00 |
| PRDM2 | 0.00 | 0.00 | 0.00 | 0.00 | 0.00 | 0.00 | 3.91 | 0.00 | 3.48 | 0.00 | 0.00 | 0.00 |
| PRDX6 | 0.00 | 0.00 | 0.00 | 0.00 | 0.00 | 0.00 | 0.00 | 0.00 | 0.00 | 0.00 | 0.00 | 3.84 |
| PRELID2 | 0.00 | 0.00 | 0.00 | 0.00 | 0.00 | 0.00 | 0.00 | 0.00 | 0.00 | 0.00 | 0.00 | 0.00 |
| PRF1 | 0.00 | 0.00 | 0.00 | 0.00 | 5.82 | 0.00 | 0.00 | 0.00 | 0.00 | 0.00 | 0.00 | 0.00 |
| PRKX | 0.00 | 0.00 | 0.00 | 0.00 | 0.00 | 0.00 | 2.98 | 0.00 | 0.00 | 0.00 | 0.00 | 0.00 |
| PRR5 | 0.00 | 0.00 | 0.00 | 0.00 | 0.00 | 0.00 | 0.00 | 0.00 | 0.00 | 0.00 | 0.00 | 0.00 |
| PRR5L | 0.00 | 0.00 | 0.00 | 0.00 | 0.00 | 0.00 | 3.05 | 0.00 | 0.00 | 0.00 | 0.00 | 0.00 |
| PSAP | 0.00 | 0.00 | 0.00 | 0.00 | 0.00 | 0.00 | 5.74 | 0.00 | 0.00 | 0.00 | 0.00 | 0.00 |
| PSMB10 | 0.00 | 3.08 | 0.00 | 0.00 | 0.00 | 0.00 | 4.11 | 0.00 | 6.27 | 0.00 | 0.00 | 3.29 |
| PSMB8 | 0.00 | 4.41 | 4.15 | 0.00 | 0.00 | 0.00 | 4.08 | 0.00 | 4.75 | 0.00 | 0.00 | 0.00 |
| PSMB9 | 0.00 | 3.15 | 0.00 | 0.00 | 0.00 | 0.00 | 4.25 | 0.00 | 4.51 | 0.00 | 0.00 | 0.00 |
| PSME1 | 0.00 | 4.01 | 0.00 | 0.00 | 0.00 | 0.00 | 3.35 | 0.00 | 3.35 | 4.18 | 0.00 | 0.00 |
| PSME2 | 0.00 | 0.00 | 0.00 | 0.00 | 0.00 | 0.00 | 3.87 | 0.00 | 3.28 | 5.52 | 0.00 | 0.00 |
| PSME2B | 0.00 | 0.00 | 0.00 | 0.00 | 0.00 | 0.00 | 0.00 | 0.00 | 0.00 | 0.00 | 0.00 | 0.00 |
| PTAFR | 0.00 | 0.00 | 0.00 | 0.00 | 0.00 | 3.86 | 0.00 | 0.00 | 0.00 | 0.00 | 0.00 | 5.08 |
| PTGDS | 0.00 | 0.00 | 0.00 | 0.00 | 0.00 | 0.00 | 0.00 | 0.00 | 0.00 | 0.00 | 0.00 | 0.00 |
| PTGS2 | 0.00 | 0.00 | 0.00 | 0.00 | 0.00 | 4.98 | 0.00 | 0.00 | 0.00 | 0.00 | 0.00 | 8.63 |
| PTP4A1 | 6.31 | 0.00 | 0.00 | 0.00 | 0.00 | 0.00 | 0.00 | 0.00 | 0.00 | 0.00 | 0.00 | 5.04 |
| PTPN18 | 0.00 | 0.00 | 0.00 | 0.00 | 0.00 | 0.00 | 0.00 | 0.00 | 3.52 | 0.00 | 0.00 | 0.00 |
| PTPN22 | 8.53 | 0.00 | 0.00 | 0.00 | 0.00 | 0.00 | 3.86 | 0.00 | 0.00 | 0.00 | 0.00 | 0.00 |
| PTPRC | 0.00 | 3.23 | 0.00 | 0.00 | 0.00 | 0.00 | 0.00 | 0.00 | 0.00 | 0.00 | 0.00 | 0.00 |
| PTPRE | 0.00 | 0.00 | 0.00 | 0.00 | 0.00 | 4.63 | 0.00 | 0.00 | 0.00 | 0.00 | 0.00 | 0.00 |
| QTRTD1 | 0.00 | 0.00 | 0.00 | 0.00 | 0.00 | 0.00 | 0.00 | 4.11 | 0.00 | 0.00 | 0.00 | 0.00 |
| RAB7B | 0.00 | 0.00 | 0.00 | 0.00 | 0.00 | 0.00 | 4.13 | 0.00 | 0.00 | 0.00 | 0.00 | 0.00 |
| RACGAP1 | 0.00 | 0.00 | 0.00 | 0.00 | 0.00 | 0.00 | 4.69 | 0.00 | 0.00 | 0.00 | 0.00 | 0.00 |
| RAMP1 | 0.00 | 0.00 | 0.00 | 0.00 | 0.00 | 0.00 | 0.00 | 0.00 | 0.00 | 0.00 | 0.00 | 0.00 |
| RAP1B | 0.00 | 0.00 | 0.00 | 0.00 | 0.00 | 0.00 | 0.00 | 0.00 | 0.00 | 0.00 | 0.00 | 0.00 |
| RAPGEF2 | 0.00 | 0.00 | 0.00 | 0.00 | 0.00 | 0.00 | 0.00 | 0.00 | 3.32 | 0.00 | 0.00 | 0.00 |
| RAPGEF6 | 0.00 | 0.00 | 0.00 | 0.00 | 0.00 | 0.00 | 0.00 | 0.00 | 0.00 | 0.00 | 0.00 | 0.00 |
| RASA4 | 0.00 | 0.00 | 0.00 | 0.00 | 0.00 | 0.00 | 4.27 | 0.00 | 3.43 | 0.00 | 0.00 | 0.00 |
| RBM38 | 0.00 | 0.00 | 0.00 | 0.00 | 0.00 | 0.00 | 0.00 | 0.00 | 0.00 | 0.00 | 0.00 | 0.00 |
| RBM45 | 0.00 | 0.00 | 0.00 | 0.00 | 4.16 | 0.00 | 0.00 | 0.00 | 0.00 | 0.00 | 0.00 | 0.00 |
| RBPJ | 0.00 | 0.00 | 4.30 | 0.00 | 0.00 | 0.00 | 0.00 | 0.00 | 0.00 | 0.00 | 0.00 | 0.00 |
| RCHY1 | 0.00 | 0.00 | 0.00 | 0.00 | 0.00 | 0.00 | 0.00 | 0.00 | 0.00 | 0.00 | 0.00 | 0.00 |
| REL | 0.00 | 0.00 | 0.00 | 0.00 | 0.00 | 0.00 | 4.33 | 0.00 | 0.00 | 0.00 | 0.00 | 0.00 |
| RETNLG | 24.05 | 9.28 | 0.00 | 0.00 | 0.00 | 19.48 | 4.64 | 17.18 | 6.79 | 0.00 | 12.85 | 92.48 |
| RGCC | 0.00 | 0.00 | 0.00 | 0.00 | 0.00 | 0.00 | 0.00 | 0.00 | 0.00 | 0.00 | 0.00 | 9.66 |
| RGL1 | 0.00 | 3.92 | 0.00 | 0.00 | 0.00 | 0.00 | 0.00 | 0.00 | 3.26 | 0.00 | 0.00 | 0.00 |
| RGS1 | 0.00 | 0.00 | 0.00 | 0.00 | 0.00 | 0.00 | 0.00 | 0.00 | 0.00 | 0.00 | 0.00 | 0.00 |
| RHOV | 0.00 | 0.00 | 0.00 | 0.00 | 0.00 | 0.00 | 0.00 | 4.53 | 5.42 | 0.00 | 0.00 | 7.58 |
| RIN2 | 0.00 | 4.91 | 0.00 | 0.00 | 0.00 | 0.00 | 0.00 | 0.00 | 0.00 | 0.00 | 0.00 | 0.00 |
| RINL | 0.00 | 0.00 | 0.00 | 0.00 | 0.00 | 0.00 | 0.00 | 0.00 | 0.00 | 0.00 | 0.00 | 0.00 |
| RNASE6 | 0.00 | 0.00 | 0.00 | 0.00 | 0.00 | 0.00 | 0.00 | 0.00 | 0.00 | 0.00 | 0.00 | 0.00 |
| RNF138 | 0.00 | 0.00 | 0.00 | 0.00 | 0.00 | 0.00 | 0.00 | 0.00 | 0.00 | 0.00 | 0.00 | 0.00 |
| RNF213 | 0.00 | 3.20 | 0.00 | 0.00 | 0.00 | 0.00 | 0.00 | 0.00 | 5.13 | 0.00 | 0.00 | 0.00 |
| RNF219 | 0.00 | 0.00 | 0.00 | 0.00 | 0.00 | 0.00 | 0.00 | 0.00 | 0.00 | 0.00 | 0.00 | 0.00 |
| ROCK2 | 0.00 | 0.00 | 0.00 | 0.00 | 0.00 | 0.00 | 0.00 | 0.00 | 0.00 | 0.00 | 0.00 | 0.00 |
| RP23-6I17.1 | 0.00 | 0.00 | 0.00 | 0.00 | 0.00 | 0.00 | 0.00 | 10.72 | 0.00 | 0.00 | 0.00 | 5.60 |
| RPL13-PS3 | 3.84 | 0.00 | 0.00 | 0.00 | 0.00 | 0.00 | 0.00 | 0.00 | 0.00 | 0.00 | 0.00 | 0.00 |
| RPL22L1 | 0.00 | 0.00 | 0.00 | 0.00 | 0.00 | 0.00 | 3.46 | 0.00 | 0.00 | 0.00 | 0.00 | 0.00 |
| RPL35 | 4.33 | 0.00 | 0.00 | 0.00 | 0.00 | 0.00 | 0.00 | 0.00 | 0.00 | 0.00 | 0.00 | 0.00 |
| RPL36-PS3 | 3.85 | 0.00 | 0.00 | 0.00 | 0.00 | 0.00 | 0.00 | 0.00 | 0.00 | 0.00 | 0.00 | 0.00 |
| RPS10 | 0.00 | 0.00 | 0.00 | 0.00 | 4.14 | 0.00 | 0.00 | 0.00 | 0.00 | 0.00 | 0.00 | 0.00 |
| RPS18-PS3 | 4.76 | 0.00 | 0.00 | 0.00 | 0.00 | 0.00 | 0.00 | 0.00 | 0.00 | 0.00 | 0.00 | 0.00 |
| RRBP1 | 0.00 | 3.16 | 0.00 | 0.00 | 0.00 | 0.00 | 0.00 | 0.00 | 0.00 | 0.00 | 0.00 | 0.00 |
| RRM2 | 0.00 | 0.00 | 0.00 | 0.00 | 0.00 | 0.00 | 0.00 | 0.00 | 4.32 | 0.00 | 0.00 | 0.00 |
| RSAD2 | 0.00 | 0.00 | 0.00 | 0.00 | 0.00 | 0.00 | 4.22 | 0.00 | 0.00 | 8.58 | 0.00 | 0.00 |
| RSRP1 | 0.00 | 0.00 | 0.00 | 0.00 | 0.00 | 0.00 | 0.00 | 3.69 | 0.00 | 0.00 | 0.00 | 0.00 |
| RTN4IP1 | 0.00 | 0.00 | 0.00 | 0.00 | 0.00 | 0.00 | 0.00 | 4.30 | 0.00 | 0.00 | 0.00 | 0.00 |
| RTP4 | 0.00 | 5.60 | 0.00 | 0.00 | 0.00 | 0.00 | 9.45 | 0.00 | 6.49 | 0.00 | 0.00 | 0.00 |
| RUNX3 | 0.00 | 0.00 | 0.00 | 0.00 | 0.00 | 0.00 | 0.00 | 0.00 | 0.00 | 0.00 | 0.00 | 0.00 |
| S100A11 | 0.00 | 0.00 | 0.00 | 0.00 | 4.52 | 0.00 | 3.06 | 0.00 | 0.00 | 0.00 | 0.00 | 7.66 |
| S100A13 | 3.54 | 0.00 | 0.00 | 0.00 | 0.00 | 0.00 | 0.00 | 0.00 | 0.00 | 0.00 | 0.00 | 0.00 |
| S100A4 | 0.00 | 0.00 | 0.00 | 0.00 | 3.75 | 0.00 | 0.00 | 0.00 | 0.00 | 0.00 | 0.00 | 0.00 |
| S100A6 | 0.00 | 0.00 | 0.00 | 0.00 | 5.21 | 0.00 | 0.00 | 0.00 | 0.00 | 0.00 | 0.00 | 0.00 |
| S100A9 | 32.86 | 4.22 | 4.31 | 0.00 | 5.68 | 28.94 | 5.13 | 22.21 | 4.78 | 6.73 | 16.14 | 123.00 |
| S1PR1 | 0.00 | 0.00 | 0.00 | 0.00 | 0.00 | 0.00 | 0.00 | 0.00 | 0.00 | 0.00 | 0.00 | 0.00 |
| SAMHD1 | 0.00 | 3.85 | 0.00 | 0.00 | 0.00 | 0.00 | 6.21 | 0.00 | 3.32 | 4.93 | 0.00 | 0.00 |
| SAP30L | 0.00 | 0.00 | 0.00 | 0.00 | 0.00 | 0.00 | 0.00 | 0.00 | 0.00 | 0.00 | 0.00 | 0.00 |
| SAT1 | 3.70 | 0.00 | 0.00 | 0.00 | 0.00 | 0.00 | 0.00 | 0.00 | 0.00 | 0.00 | 3.88 | 0.00 |
| SDC3 | 0.00 | 5.61 | 0.00 | 0.00 | 0.00 | 0.00 | 4.18 | 0.00 | 6.61 | 0.00 | 0.00 | 0.00 |
| SDC4 | 0.00 | 0.00 | 0.00 | 0.00 | 0.00 | 0.00 | 0.00 | 0.00 | 0.00 | 0.00 | 0.00 | 0.00 |
| SDCBP | 0.00 | 0.00 | 0.00 | 0.00 | 0.00 | 0.00 | 3.24 | 0.00 | 0.00 | 0.00 | 0.00 | 0.00 |
| SDF4 | 0.00 | 0.00 | 0.00 | 0.00 | 0.00 | 0.00 | 0.00 | 0.00 | 0.00 | 0.00 | 0.00 | 0.00 |
| SDHAF1 | 3.62 | 0.00 | 0.00 | 0.00 | 0.00 | 0.00 | 0.00 | 0.00 | 0.00 | 0.00 | 0.00 | 0.00 |
| SELT | 0.00 | 0.00 | 0.00 | 0.00 | 0.00 | 0.00 | 0.00 | 0.00 | 0.00 | 0.00 | 0.00 | 0.00 |
| SENP6 | 0.00 | 0.00 | 0.00 | 0.00 | 0.00 | 0.00 | 0.00 | 0.00 | 0.00 | 0.00 | 0.00 | 0.00 |
| SEPN1 | 0.00 | 0.00 | 0.00 | 0.00 | 0.00 | 0.00 | 0.00 | 0.00 | 0.00 | 0.00 | 0.00 | 0.00 |
| SEPP1 | 0.00 | 3.02 | 0.00 | 0.00 | 0.00 | 0.00 | 0.00 | 0.00 | 4.43 | 0.00 | 0.00 | 0.00 |
| SEPT11 | 0.00 | 0.00 | 0.00 | 0.00 | 0.00 | 0.00 | 0.00 | 0.00 | 0.00 | 0.00 | 0.00 | 0.00 |
| SEPW1 | 0.00 | 0.00 | 0.00 | 0.00 | 0.00 | 0.00 | 3.20 | 0.00 | 0.00 | 0.00 | 0.00 | 0.00 |
| SERPINA3F | 0.00 | 0.00 | 0.00 | 0.00 | 0.00 | 0.00 | 0.00 | 0.00 | 0.00 | 0.00 | 0.00 | 0.00 |
| SERPINA3G | 0.00 | 0.00 | 0.00 | 0.00 | 0.00 | 0.00 | 0.00 | 0.00 | 0.00 | 0.00 | 0.00 | 0.00 |
| SERPINB1A | 0.00 | 0.00 | 0.00 | 0.00 | 0.00 | 0.00 | 0.00 | 0.00 | 0.00 | 0.00 | 0.00 | 4.63 |
| SERPINH1 | 0.00 | 0.00 | 4.00 | 0.00 | 0.00 | 0.00 | 0.00 | 0.00 | 0.00 | 0.00 | 0.00 | 0.00 |
| SERTAD1 | 0.00 | 0.00 | 0.00 | 0.00 | 0.00 | 0.00 | 0.00 | 0.00 | 0.00 | 0.00 | 0.00 | 4.48 |
| SETX | 0.00 | 0.00 | 0.00 | 0.00 | 0.00 | 0.00 | 0.00 | 0.00 | 0.00 | 0.00 | 0.00 | 0.00 |
| SFT2D1 | 0.00 | 0.00 | 0.00 | 0.00 | 0.00 | 0.00 | 0.00 | 0.00 | 0.00 | 0.00 | 0.00 | 0.00 |
| SGK3 | 0.00 | 0.00 | 0.00 | 0.00 | 0.00 | 0.00 | 0.00 | 0.00 | 0.00 | 0.00 | 0.00 | 0.00 |
| SGMS2 | 0.00 | 0.00 | 0.00 | 0.00 | 0.00 | 0.00 | 0.00 | 0.00 | 0.00 | 0.00 | 0.00 | 4.73 |
| SH2B2 | 0.00 | 0.00 | 0.00 | 0.00 | 0.00 | 0.00 | 0.00 | 0.00 | 0.00 | 0.00 | 0.00 | 0.00 |
| SHISA5 | 0.00 | 0.00 | 0.00 | 0.00 | 0.00 | 0.00 | 0.00 | 0.00 | 0.00 | 0.00 | 0.00 | 0.00 |
| SIPA1L1 | 0.00 | 0.00 | 0.00 | 0.00 | 0.00 | 0.00 | 0.00 | 0.00 | 0.00 | 0.00 | 0.00 | 0.00 |
| SLAMF7 | 0.00 | 0.00 | 0.00 | 0.00 | 4.09 | 0.00 | 0.00 | 3.61 | 0.00 | 0.00 | 0.00 | 0.00 |
| SLAMF8 | 0.00 | 0.00 | 0.00 | 0.00 | 0.00 | 0.00 | 3.32 | 0.00 | 0.00 | 0.00 | 0.00 | 0.00 |
| SLAMF9 | 0.00 | 3.61 | 0.00 | 0.00 | 0.00 | 0.00 | 0.00 | 0.00 | 0.00 | 0.00 | 0.00 | 0.00 |
| SLC12A9 | 0.00 | 3.29 | 0.00 | 0.00 | 0.00 | 0.00 | 0.00 | 0.00 | 4.12 | 0.00 | 0.00 | 0.00 |
| SLC17A5 | 0.00 | 0.00 | 0.00 | 0.00 | 0.00 | 0.00 | 0.00 | 0.00 | 0.00 | 0.00 | 0.00 | 0.00 |
| SLC26A2 | 0.00 | 0.00 | 0.00 | 0.00 | 0.00 | 0.00 | 2.99 | 0.00 | 0.00 | 0.00 | 0.00 | 0.00 |
| SLC28A2 | 0.00 | 0.00 | 0.00 | 0.00 | 0.00 | 0.00 | 0.00 | 0.00 | 6.27 | 0.00 | 0.00 | 0.00 |
| SLC2A6 | 0.00 | 0.00 | 0.00 | 0.00 | 0.00 | 0.00 | 0.00 | 0.00 | 3.16 | 0.00 | 0.00 | 0.00 |
| SLC30A1 | 0.00 | 0.00 | 0.00 | 0.00 | 0.00 | 0.00 | 3.46 | 0.00 | 0.00 | 0.00 | 0.00 | 0.00 |
| SLC35D1 | 0.00 | 0.00 | 0.00 | 0.00 | 0.00 | 0.00 | 0.00 | 0.00 | 0.00 | 0.00 | 0.00 | 0.00 |
| SLC36A3OS | 0.00 | 0.00 | 0.00 | 0.00 | 0.00 | 0.00 | 0.00 | 0.00 | 0.00 | 0.00 | 0.00 | 0.00 |
| SLC38A6 | 0.00 | 0.00 | 0.00 | 0.00 | 0.00 | 0.00 | 0.00 | 0.00 | 0.00 | 0.00 | 0.00 | 0.00 |
| SLC45A2 | 0.00 | 3.03 | 0.00 | 0.00 | 0.00 | 0.00 | 0.00 | 0.00 | 0.00 | 0.00 | 0.00 | 0.00 |
| SLC7A11 | 0.00 | 7.49 | 0.00 | 0.00 | 0.00 | 0.00 | 0.00 | 0.00 | 6.91 | 0.00 | 0.00 | 10.88 |
| SLFN1 | 0.00 | 4.50 | 0.00 | 0.00 | 0.00 | 0.00 | 5.93 | 0.00 | 0.00 | 0.00 | 0.00 | 0.00 |
| SLFN2 | 0.00 | 4.69 | 0.00 | 0.00 | 0.00 | 0.00 | 6.75 | 0.00 | 4.22 | 0.00 | 0.00 | 0.00 |
| SLFN5 | 0.00 | 5.79 | 5.27 | 0.00 | 0.00 | 0.00 | 4.66 | 0.00 | 0.00 | 0.00 | 5.78 | 0.00 |
| SLFN8 | 0.00 | 0.00 | 0.00 | 0.00 | 0.00 | 0.00 | 0.00 | 0.00 | 3.69 | 0.00 | 0.00 | 0.00 |
| SLFN9 | 0.00 | 0.00 | 0.00 | 0.00 | 0.00 | 0.00 | 0.00 | 0.00 | 0.00 | 3.61 | 0.00 | 0.00 |
| SLK | 0.00 | 0.00 | 0.00 | 0.00 | 4.29 | 0.00 | 3.05 | 0.00 | 0.00 | 0.00 | 0.00 | 0.00 |
| SLPI | 5.75 | 0.00 | 0.00 | 0.00 | 0.00 | 0.00 | 0.00 | 0.00 | 4.36 | 0.00 | 0.00 | 15.50 |
| SMIM14 | 0.00 | 0.00 | 0.00 | 0.00 | 0.00 | 0.00 | 0.00 | 0.00 | 0.00 | 0.00 | 0.00 | 0.00 |
| SMOX | 0.00 | 0.00 | 0.00 | 0.00 | 0.00 | 0.00 | 0.00 | 0.00 | 0.00 | 0.00 | 0.00 | 5.16 |
| SNN | 0.00 | 0.00 | 0.00 | 0.00 | 0.00 | 0.00 | 0.00 | 0.00 | 0.00 | 0.00 | 0.00 | 0.00 |
| SOCS1 | 0.00 | 4.96 | 0.00 | 0.00 | 0.00 | 0.00 | 0.00 | 0.00 | 0.00 | 0.00 | 0.00 | 0.00 |
| SOCS3 | 0.00 | 0.00 | 0.00 | 0.00 | 0.00 | 0.00 | 0.00 | 0.00 | 0.00 | 0.00 | 0.00 | 0.00 |
| SON | 3.85 | 0.00 | 0.00 | 0.00 | 0.00 | 3.89 | 0.00 | 0.00 | 0.00 | 0.00 | 0.00 | 0.00 |
| SOX4 | 0.00 | 0.00 | 0.00 | 0.00 | 5.58 | 0.00 | 0.00 | 0.00 | 0.00 | 0.00 | 0.00 | 0.00 |
| SP100 | 0.00 | 3.07 | 0.00 | 0.00 | 0.00 | 0.00 | 0.00 | 0.00 | 0.00 | 0.00 | 0.00 | 0.00 |
| SP110 | 0.00 | 4.88 | 0.00 | 0.00 | 0.00 | 0.00 | 7.06 | 0.00 | 0.00 | 0.00 | 0.00 | 0.00 |
| SP140 | 0.00 | 0.00 | 0.00 | 0.00 | 0.00 | 0.00 | 3.28 | 0.00 | 3.51 | 5.82 | 0.00 | 0.00 |
| SPI1 | 0.00 | 0.00 | 0.00 | 0.00 | 0.00 | 0.00 | 3.89 | 0.00 | 0.00 | 0.00 | 0.00 | 0.00 |
| SPIN2C | 0.00 | 0.00 | 0.00 | 0.00 | 0.00 | 0.00 | 0.00 | 0.00 | 0.00 | 0.00 | 0.00 | 0.00 |
| SPINT1 | 0.00 | 3.78 | 3.92 | 0.00 | 0.00 | 0.00 | 0.00 | 0.00 | 3.50 | 0.00 | 0.00 | 0.00 |
| SPP1 | 0.00 | 0.00 | 0.00 | 0.00 | 0.00 | 0.00 | 0.00 | 0.00 | 0.00 | 0.00 | 4.25 | 22.97 |
| SPRED1 | 0.00 | 0.00 | 0.00 | 0.00 | 0.00 | 0.00 | 3.82 | 0.00 | 0.00 | 0.00 | 0.00 | 0.00 |
| SPRY2 | 0.00 | 0.00 | 0.00 | 0.00 | 0.00 | 0.00 | 0.00 | 0.00 | 0.00 | 0.00 | 0.00 | 4.07 |
| SRGAP3 | 0.00 | 0.00 | 0.00 | 0.00 | 0.00 | 0.00 | 0.00 | 0.00 | 3.49 | 0.00 | 0.00 | 0.00 |
| SRGN | 0.00 | 0.00 | 0.00 | 0.00 | 0.00 | 0.00 | 0.00 | 0.00 | 0.00 | 0.00 | 0.00 | 5.72 |
| SRRM1 | 0.00 | 0.00 | 0.00 | 0.00 | 0.00 | 0.00 | 4.32 | 0.00 | 0.00 | 0.00 | 0.00 | 0.00 |
| SRSF5 | 3.56 | 0.00 | 0.00 | 0.00 | 0.00 | 0.00 | 0.00 | 0.00 | 0.00 | 0.00 | 0.00 | 0.00 |
| ST13 | 4.24 | 0.00 | 0.00 | 0.00 | 0.00 | 0.00 | 0.00 | 0.00 | 0.00 | 0.00 | 0.00 | 0.00 |
| ST3GAL6 | 0.00 | 0.00 | 0.00 | 0.00 | 0.00 | 0.00 | 0.00 | 0.00 | 0.00 | 0.00 | 0.00 | 5.39 |
| STAT2 | 0.00 | 5.46 | 0.00 | 0.00 | 0.00 | 0.00 | 0.00 | 4.14 | 4.16 | 0.00 | 0.00 | 0.00 |
| STX11 | 0.00 | 0.00 | 0.00 | 0.00 | 0.00 | 0.00 | 0.00 | 0.00 | 0.00 | 0.00 | 0.00 | 0.00 |
| SUB1 | 0.00 | 0.00 | 0.00 | 0.00 | 0.00 | 0.00 | 0.00 | 0.00 | 0.00 | 4.34 | 0.00 | 0.00 |
| SYNE1 | 0.00 | 0.00 | 0.00 | 0.00 | 0.00 | 0.00 | 0.00 | 0.00 | 0.00 | 0.00 | 0.00 | 4.21 |
| SYNJ1 | 0.00 | 3.70 | 0.00 | 0.00 | 0.00 | 0.00 | 0.00 | 0.00 | 0.00 | 0.00 | 0.00 | 0.00 |
| TAF15 | 0.00 | 0.00 | 0.00 | 0.00 | 0.00 | 0.00 | 0.00 | 0.00 | 0.00 | 0.00 | 0.00 | 0.00 |
| TALDO1 | 0.00 | 0.00 | 0.00 | 0.00 | 0.00 | 0.00 | 3.10 | 0.00 | 0.00 | 0.00 | 0.00 | 0.00 |
| TAP1 | 0.00 | 6.12 | 0.00 | 0.00 | 0.00 | 0.00 | 3.83 | 0.00 | 3.87 | 3.68 | 0.00 | 0.00 |
| TAPBP | 0.00 | 0.00 | 0.00 | 0.00 | 0.00 | 0.00 | 3.10 | 0.00 | 4.30 | 0.00 | 0.00 | 0.00 |
| TAPBPL | 0.00 | 3.24 | 0.00 | 0.00 | 0.00 | 0.00 | 0.00 | 0.00 | 0.00 | 0.00 | 0.00 | 0.00 |
| TBX21 | 0.00 | 0.00 | 0.00 | 0.00 | 0.00 | 0.00 | 0.00 | 0.00 | 0.00 | 0.00 | 0.00 | 0.00 |
| TBXA2R | 0.00 | 0.00 | 0.00 | 0.00 | 0.00 | 0.00 | 3.11 | 0.00 | 0.00 | 0.00 | 0.00 | 0.00 |
| TCP11L2 | 0.00 | 0.00 | 0.00 | 0.00 | 0.00 | 0.00 | 0.00 | 0.00 | 0.00 | 0.00 | 0.00 | 0.00 |
| TDRD7 | 0.00 | 3.27 | 0.00 | 0.00 | 0.00 | 0.00 | 0.00 | 0.00 | 0.00 | 0.00 | 0.00 | 0.00 |
| TECPR1 | 0.00 | 0.00 | 0.00 | 0.00 | 0.00 | 0.00 | 4.47 | 0.00 | 0.00 | 0.00 | 0.00 | 0.00 |
| TESC | 0.00 | 0.00 | 0.00 | 0.00 | 0.00 | 0.00 | 3.39 | 0.00 | 0.00 | 0.00 | 0.00 | 0.00 |
| TEX30 | 0.00 | 0.00 | 0.00 | 0.00 | 0.00 | 0.00 | 0.00 | 0.00 | 0.00 | 0.00 | 0.00 | 0.00 |
| TGTP1 | 0.00 | 0.00 | 0.00 | 0.00 | 0.00 | 0.00 | 0.00 | 0.00 | 0.00 | 5.93 | 0.00 | 0.00 |
| TGTP2 | 0.00 | 7.88 | 4.35 | 0.00 | 0.00 | 0.00 | 5.42 | 0.00 | 5.99 | 6.73 | 4.62 | 0.00 |
| THBS1 | 0.00 | 0.00 | 0.00 | 0.00 | 0.00 | 0.00 | 0.00 | 0.00 | 3.37 | 0.00 | 0.00 | 16.16 |
| THEMIS2 | 0.00 | 0.00 | 0.00 | 0.00 | 0.00 | 0.00 | 4.59 | 0.00 | 5.87 | 0.00 | 0.00 | 0.00 |
| TIMP2 | 0.00 | 0.00 | 0.00 | 0.00 | 0.00 | 0.00 | 3.83 | 0.00 | 0.00 | 0.00 | 0.00 | 0.00 |
| TIPARP | 0.00 | 0.00 | 0.00 | 0.00 | 0.00 | 0.00 | 0.00 | 0.00 | 0.00 | 0.00 | 0.00 | 4.47 |
| TLE3 | 0.00 | 0.00 | 0.00 | 0.00 | 0.00 | 0.00 | 3.46 | 0.00 | 0.00 | 0.00 | 0.00 | 0.00 |
| TLK1 | 0.00 | 0.00 | 0.00 | 0.00 | 0.00 | 0.00 | 0.00 | 0.00 | 0.00 | 0.00 | 0.00 | 0.00 |
| TMEM173 | 0.00 | 0.00 | 0.00 | 0.00 | 0.00 | 0.00 | 0.00 | 0.00 | 0.00 | 0.00 | 0.00 | 0.00 |
| TMEM176A | 0.00 | 3.58 | 0.00 | 0.00 | 0.00 | 0.00 | 0.00 | 0.00 | 0.00 | 0.00 | 0.00 | 0.00 |
| TMEM176B | 0.00 | 6.05 | 0.00 | 0.00 | 0.00 | 0.00 | 3.66 | 4.33 | 0.00 | 0.00 | 0.00 | 0.00 |
| TMEM229B | 0.00 | 0.00 | 0.00 | 0.00 | 0.00 | 0.00 | 0.00 | 0.00 | 0.00 | 0.00 | 0.00 | 0.00 |
| TMSB10 | 0.00 | 0.00 | 0.00 | 0.00 | 0.00 | 0.00 | 6.56 | 0.00 | 0.00 | 0.00 | 0.00 | 0.00 |
| TMSB4X | 0.00 | 0.00 | 0.00 | 0.00 | 4.13 | 0.00 | 3.90 | 0.00 | 0.00 | 3.63 | 0.00 | 0.00 |
| TMX4 | 0.00 | 0.00 | 0.00 | 0.00 | 0.00 | 0.00 | 0.00 | 0.00 | 0.00 | 0.00 | 0.00 | 0.00 |
| TNFAIP3 | 0.00 | 0.00 | 0.00 | 0.00 | 0.00 | 0.00 | 0.00 | 0.00 | 0.00 | 0.00 | 0.00 | 0.00 |
| TNFRSF18 | 0.00 | 0.00 | 0.00 | 0.00 | 0.00 | 0.00 | 0.00 | 0.00 | 0.00 | 0.00 | 0.00 | 0.00 |
| TNFRSF4 | 0.00 | 0.00 | 0.00 | 0.00 | 0.00 | 0.00 | 0.00 | 0.00 | 0.00 | 0.00 | 0.00 | 0.00 |
| TNFRSF9 | 0.00 | 0.00 | 0.00 | 0.00 | 0.00 | 0.00 | 0.00 | 3.67 | 0.00 | 0.00 | 0.00 | 0.00 |
| TNFSF10 | 0.00 | 4.03 | 0.00 | 0.00 | 0.00 | 0.00 | 0.00 | 0.00 | 0.00 | 0.00 | 0.00 | 0.00 |
| TNIP3 | 0.00 | 0.00 | 0.00 | 0.00 | 0.00 | 0.00 | 3.27 | 0.00 | 0.00 | 0.00 | 0.00 | 0.00 |
| TNNI2 | 0.00 | 0.00 | 0.00 | 0.00 | 0.00 | 0.00 | 0.00 | 0.00 | 0.00 | 3.78 | 0.00 | 0.00 |
| TOB1 | 4.43 | 0.00 | 0.00 | 0.00 | 0.00 | 0.00 | 0.00 | 0.00 | 0.00 | 0.00 | 0.00 | 0.00 |
| TOMM34 | 0.00 | 0.00 | 0.00 | 0.00 | 0.00 | 0.00 | 0.00 | 0.00 | 0.00 | 0.00 | 0.00 | 3.55 |
| TOP2A | 0.00 | 0.00 | 0.00 | 0.00 | 0.00 | 0.00 | 0.00 | 0.00 | 0.00 | 0.00 | 0.00 | 0.00 |
| TOPBP1 | 0.00 | 0.00 | 0.00 | 0.00 | 0.00 | 0.00 | 0.00 | 0.00 | 0.00 | 0.00 | 0.00 | 0.00 |
| TOR1AIP1 | 0.00 | 5.62 | 0.00 | 0.00 | 0.00 | 0.00 | 3.28 | 0.00 | 0.00 | 0.00 | 0.00 | 0.00 |
| TOR3A | 0.00 | 4.12 | 0.00 | 0.00 | 0.00 | 3.67 | 0.00 | 0.00 | 0.00 | 0.00 | 0.00 | 0.00 |
| TPM3-RS7 | 0.00 | 0.00 | 6.65 | 0.00 | 0.00 | 0.00 | 0.00 | 0.00 | 0.00 | 0.00 | 0.00 | 0.00 |
| TPM4 | 0.00 | 0.00 | 0.00 | 0.00 | 0.00 | 0.00 | 0.00 | 0.00 | 0.00 | 0.00 | 0.00 | 0.00 |
| TPST1 | 0.00 | 0.00 | 0.00 | 0.00 | 0.00 | 0.00 | 3.10 | 0.00 | 0.00 | 0.00 | 0.00 | 0.00 |
| TRA2B | 5.00 | 0.00 | 0.00 | 0.00 | 3.80 | 6.79 | 0.00 | 0.00 | 3.71 | 0.00 | 0.00 | 0.00 |
| TRAF1 | 0.00 | 0.00 | 0.00 | 0.00 | 0.00 | 0.00 | 0.00 | 0.00 | 0.00 | 0.00 | 0.00 | 0.00 |
| TRAFD1 | 0.00 | 0.00 | 0.00 | 0.00 | 0.00 | 0.00 | 4.30 | 0.00 | 0.00 | 0.00 | 0.00 | 0.00 |
| TREM1 | 0.00 | 0.00 | 0.00 | 0.00 | 0.00 | 4.76 | 0.00 | 0.00 | 3.61 | 0.00 | 4.46 | 7.63 |
| TRIM12C | 0.00 | 3.57 | 0.00 | 0.00 | 0.00 | 0.00 | 3.52 | 0.00 | 0.00 | 0.00 | 0.00 | 0.00 |
| TRIM14 | 0.00 | 4.30 | 0.00 | 0.00 | 0.00 | 0.00 | 0.00 | 0.00 | 0.00 | 0.00 | 0.00 | 0.00 |
| TRIM30A | 0.00 | 0.00 | 0.00 | 0.00 | 0.00 | 0.00 | 0.00 | 0.00 | 0.00 | 0.00 | 0.00 | 0.00 |
| TRIM30B | 0.00 | 3.46 | 0.00 | 0.00 | 0.00 | 0.00 | 3.59 | 0.00 | 0.00 | 0.00 | 0.00 | 0.00 |
| TRIM30C | 0.00 | 0.00 | 0.00 | 0.00 | 0.00 | 0.00 | 5.29 | 0.00 | 0.00 | 0.00 | 0.00 | 0.00 |
| TRIM30D | 0.00 | 0.00 | 0.00 | 0.00 | 0.00 | 0.00 | 0.00 | 0.00 | 3.38 | 0.00 | 0.00 | 0.00 |
| TRIM37 | 0.00 | 0.00 | 0.00 | 0.00 | 0.00 | 0.00 | 0.00 | 0.00 | 0.00 | 0.00 | 0.00 | 0.00 |
| TRIM5 | 0.00 | 3.48 | 0.00 | 0.00 | 0.00 | 0.00 | 0.00 | 0.00 | 0.00 | 0.00 | 0.00 | 0.00 |
| TRIM56 | 0.00 | 0.00 | 0.00 | 0.00 | 0.00 | 0.00 | 0.00 | 0.00 | 0.00 | 0.00 | 0.00 | 0.00 |
| TRIP12 | 0.00 | 0.00 | 0.00 | 0.00 | 0.00 | 0.00 | 0.00 | 0.00 | 0.00 | 0.00 | 0.00 | 0.00 |
| TROVE2 | 0.00 | 0.00 | 0.00 | 0.00 | 0.00 | 0.00 | 4.01 | 0.00 | 0.00 | 0.00 | 0.00 | 0.00 |
| TSC22D3 | 4.07 | 0.00 | 0.00 | 0.00 | 0.00 | 0.00 | 0.00 | 0.00 | 0.00 | 0.00 | 0.00 | 0.00 |
| TSPAN13 | 0.00 | 0.00 | 0.00 | 0.00 | 0.00 | 0.00 | 0.00 | 0.00 | 0.00 | 0.00 | 0.00 | 0.00 |
| TSPO | 0.00 | 0.00 | 0.00 | 0.00 | 0.00 | 0.00 | 4.20 | 0.00 | 0.00 | 5.59 | 0.00 | 0.00 |
| TSTD3 | 0.00 | 0.00 | 0.00 | 0.00 | 0.00 | 4.54 | 0.00 | 0.00 | 0.00 | 0.00 | 0.00 | 0.00 |
| TTC37 | 0.00 | 3.10 | 0.00 | 0.00 | 0.00 | 0.00 | 0.00 | 0.00 | 0.00 | 0.00 | 0.00 | 0.00 |
| TUBA4A | 0.00 | 3.33 | 0.00 | 0.00 | 0.00 | 0.00 | 0.00 | 0.00 | 0.00 | 0.00 | 0.00 | 0.00 |
| TUBB5 | 0.00 | 0.00 | 0.00 | 0.00 | 0.00 | 0.00 | 0.00 | 0.00 | 3.20 | 0.00 | 0.00 | 0.00 |
| TXK | 0.00 | 0.00 | 0.00 | 0.00 | 0.00 | 0.00 | 4.57 | 0.00 | 0.00 | 0.00 | 0.00 | 0.00 |
| TXN1 | 0.00 | 0.00 | 4.67 | 0.00 | 0.00 | 0.00 | 0.00 | 0.00 | 0.00 | 0.00 | 0.00 | 0.00 |
| TXNIP | 0.00 | 4.04 | 0.00 | 0.00 | 0.00 | 0.00 | 0.00 | 0.00 | 0.00 | 0.00 | 0.00 | 0.00 |
| TYROBP | 0.00 | 0.00 | 0.00 | 0.00 | 0.00 | 0.00 | 0.00 | 0.00 | 0.00 | 0.00 | 0.00 | 0.00 |
| TYRP1 | 0.00 | 3.44 | 0.00 | 0.00 | 0.00 | 0.00 | 0.00 | 0.00 | 0.00 | 0.00 | 0.00 | 0.00 |
| UBB | 4.51 | 0.00 | 0.00 | 0.00 | 0.00 | 0.00 | 0.00 | 0.00 | 0.00 | 0.00 | 0.00 | 0.00 |
| UBC | 6.28 | 0.00 | 0.00 | 0.00 | 0.00 | 0.00 | 0.00 | 0.00 | 0.00 | 0.00 | 0.00 | 0.00 |
| UBE2C | 0.00 | 0.00 | 0.00 | 0.00 | 0.00 | 0.00 | 4.54 | 0.00 | 0.00 | 0.00 | 0.00 | 0.00 |
| UBE2G1 | 0.00 | 0.00 | 0.00 | 0.00 | 0.00 | 0.00 | 0.00 | 0.00 | 0.00 | 0.00 | 0.00 | 0.00 |
| UBE2L6 | 0.00 | 0.00 | 0.00 | 0.00 | 0.00 | 0.00 | 3.28 | 0.00 | 4.16 | 0.00 | 0.00 | 0.00 |
| UBP1 | 0.00 | 0.00 | 0.00 | 0.00 | 0.00 | 4.16 | 0.00 | 0.00 | 0.00 | 0.00 | 0.00 | 0.00 |
| UCK2 | 0.00 | 0.00 | 0.00 | 0.00 | 0.00 | 0.00 | 0.00 | 0.00 | 0.00 | 0.00 | 0.00 | 3.43 |
| UGCG | 0.00 | 0.00 | 0.00 | 0.00 | 0.00 | 0.00 | 0.00 | 0.00 | 0.00 | 0.00 | 0.00 | 0.00 |
| UNC93B1 | 0.00 | 4.17 | 0.00 | 0.00 | 0.00 | 0.00 | 3.21 | 0.00 | 4.84 | 0.00 | 0.00 | 0.00 |
| UPF3B | 0.00 | 0.00 | 0.00 | 0.00 | 0.00 | 0.00 | 0.00 | 3.75 | 0.00 | 0.00 | 0.00 | 0.00 |
| UPP1 | 0.00 | 0.00 | 0.00 | 0.00 | 0.00 | 0.00 | 0.00 | 0.00 | 0.00 | 0.00 | 0.00 | 6.63 |
| USP18 | 0.00 | 3.18 | 0.00 | 0.00 | 0.00 | 0.00 | 0.00 | 0.00 | 0.00 | 0.00 | 0.00 | 0.00 |
| USP25 | 0.00 | 0.00 | 0.00 | 0.00 | 0.00 | 0.00 | 0.00 | 0.00 | 3.43 | 0.00 | 0.00 | 0.00 |
| USP3 | 0.00 | 0.00 | 0.00 | 0.00 | 0.00 | 0.00 | 0.00 | 0.00 | 0.00 | 0.00 | 0.00 | 0.00 |
| USP9X | 0.00 | 0.00 | 0.00 | 0.00 | 0.00 | 0.00 | 0.00 | 0.00 | 0.00 | 0.00 | 0.00 | 0.00 |
| VAV3 | 0.00 | 3.46 | 0.00 | 0.00 | 0.00 | 0.00 | 0.00 | 0.00 | 0.00 | 0.00 | 0.00 | 0.00 |
| VCPIP1 | 0.00 | 0.00 | 0.00 | 0.00 | 0.00 | 0.00 | 0.00 | 0.00 | 0.00 | 0.00 | 0.00 | 0.00 |
| VEGFA | 0.00 | 0.00 | 0.00 | 0.00 | 0.00 | 0.00 | 0.00 | 0.00 | 0.00 | 0.00 | 0.00 | 4.27 |
| VPS37B | 0.00 | 0.00 | 0.00 | 0.00 | 0.00 | 0.00 | 0.00 | 0.00 | 0.00 | 0.00 | 0.00 | 3.94 |
| XDH | 0.00 | 0.00 | 0.00 | 0.00 | 0.00 | 0.00 | 0.00 | 0.00 | 5.49 | 0.00 | 0.00 | 0.00 |
| XPR1 | 0.00 | 3.13 | 0.00 | 0.00 | 0.00 | 0.00 | 0.00 | 0.00 | 0.00 | 0.00 | 0.00 | 0.00 |
| YAF2 | 0.00 | 0.00 | 0.00 | 0.00 | 0.00 | 0.00 | 3.39 | 0.00 | 0.00 | 0.00 | 0.00 | 0.00 |
| YWHAB | 0.00 | 0.00 | 0.00 | 0.00 | 0.00 | 0.00 | 3.06 | 0.00 | 0.00 | 0.00 | 0.00 | 0.00 |
| ZC3H7A | 0.00 | 0.00 | 0.00 | 0.00 | 0.00 | 0.00 | 0.00 | 0.00 | 0.00 | 0.00 | 0.00 | 0.00 |
| ZDHHC17 | 0.00 | 0.00 | 0.00 | 0.00 | 0.00 | 0.00 | 0.00 | 0.00 | 0.00 | 0.00 | 0.00 | 0.00 |
| ZDHHC18 | 0.00 | 0.00 | 0.00 | 0.00 | 0.00 | 0.00 | 2.98 | 0.00 | 0.00 | 0.00 | 0.00 | 0.00 |
| ZFP106 | 0.00 | 3.65 | 0.00 | 0.00 | 0.00 | 0.00 | 2.98 | 0.00 | 0.00 | 0.00 | 0.00 | 0.00 |
| ZFP36L2 | 9.42 | 0.00 | 0.00 | 0.00 | 0.00 | 0.00 | 0.00 | 0.00 | 0.00 | 0.00 | 0.00 | 0.00 |
| ZFP646 | 0.00 | 0.00 | 0.00 | 0.00 | 0.00 | 0.00 | 0.00 | 0.00 | 0.00 | 0.00 | 0.00 | 0.00 |
| ZFP729A | 0.00 | 0.00 | 0.00 | 0.00 | 0.00 | 0.00 | 0.00 | 0.00 | 0.00 | 0.00 | 0.00 | 0.00 |
| ZFP954 | 0.00 | 0.00 | 0.00 | 0.00 | 0.00 | 0.00 | 0.00 | 0.00 | 0.00 | 0.00 | 0.00 | 0.00 |
| ZFX | 0.00 | 4.38 | 0.00 | 0.00 | 0.00 | 0.00 | 0.00 | 0.00 | 0.00 | 0.00 | 0.00 | 0.00 |
| ZFYVE16 | 0.00 | 0.00 | 0.00 | 0.00 | 0.00 | 0.00 | 3.65 | 0.00 | 0.00 | 0.00 | 0.00 | 0.00 |
| ZNFX1 | 0.00 | 7.79 | 0.00 | 0.00 | 0.00 | 0.00 | 0.00 | 0.00 | 3.33 | 0.00 | 0.00 | 0.00 |
| ZUFSP | 0.00 | 0.00 | 0.00 | 0.00 | 0.00 | 0.00 | 0.00 | 0.00 | 0.00 | 0.00 | 0.00 | 0.00 |

TABLE 3B
T cell clusters

| Gene | T_0 | T_1 | T_2 | T_3 | T_4 | T_5 | T_6 | T_7 | T_8 | T_9 |
|---|---|---|---|---|---|---|---|---|---|---|
| MALT1 | 0.00 | 0.00 | 0.00 | 0.00 | 4.56 | 0.00 | 0.00 | 7.59 | 0.00 | 0.00 |
| ITGA1 | 0.00 | 0.00 | 0.00 | 0.00 | 0.00 | 0.00 | 0.00 | 7.58 | 0.00 | 0.00 |
| ETNK1 | 0.00 | 0.00 | 0.00 | 0.00 | 0.00 | 0.00 | 0.00 | 7.38 | 0.00 | 0.00 |
| HBB-BS | 8.51 | 0.00 | 6.97 | 4.79 | 0.00 | 3.96 | 0.00 | 7.00 | 0.00 | 0.00 |
| SP4 | 0.00 | 0.00 | 0.00 | 0.00 | 0.00 | 0.00 | 0.00 | 6.86 | 0.00 | 0.00 |
| ATG7 | 0.00 | 0.00 | 0.00 | 0.00 | 0.00 | 0.00 | 0.00 | 6.81 | 0.00 | 0.00 |
| PYDC4 | 0.00 | 4.31 | 4.49 | 0.00 | 0.00 | 0.00 | 0.00 | 6.65 | 0.00 | 0.00 |
| PYDC3 | 0.00 | 0.00 | 0.00 | 0.00 | 0.00 | 0.00 | 0.00 | 6.48 | 0.00 | 0.00 |
| PENK | 0.00 | 0.00 | 0.00 | 0.00 | 6.09 | 0.00 | 0.00 | 6.34 | 0.00 | 5.80 |
| IFITM3 | 3.32 | 0.00 | 4.46 | 6.47 | 4.18 | 0.00 | 0.00 | 6.28 | 0.00 | 0.00 |
| SAMD9L | 0.00 | 0.00 | 0.00 | 0.00 | 3.71 | 0.00 | 0.00 | 6.12 | 0.00 | 0.00 |
| CCL8 | 0.00 | 0.00 | 3.54 | 0.00 | 0.00 | 0.00 | 0.00 | 5.92 | 0.00 | 0.00 |
| ZCCHC6 | 0.00 | 0.00 | 0.00 | 0.00 | 0.00 | 0.00 | 0.00 | 5.86 | 0.00 | 0.00 |
| C1QB | 7.18 | 0.00 | 4.22 | 0.00 | 3.33 | 3.80 | 0.00 | 5.84 | 0.00 | 0.00 |
| MIR142HG | 0.00 | 0.00 | 0.00 | 0.00 | 0.00 | 0.00 | 0.00 | 5.62 | 0.00 | 0.00 |
| PYHIN1 | 0.00 | 4.36 | 6.35 | 0.00 | 0.00 | 0.00 | 0.00 | 5.39 | 0.00 | 4.42 |
| SEPT6 | 0.00 | 0.00 | 0.00 | 0.00 | 0.00 | 0.00 | 0.00 | 5.24 | 0.00 | 0.00 |
| CCL6 | 0.00 | 0.00 | 0.00 | 0.00 | 0.00 | 0.00 | 0.00 | 5.16 | 0.00 | 0.00 |
| XAF1 | 0.00 | 0.00 | 5.19 | 0.00 | 0.00 | 0.00 | 0.00 | 5.11 | 0.00 | 0.00 |
| LGALS7 | 0.00 | 0.00 | 0.00 | 0.00 | 0.00 | 0.00 | 0.00 | 5.11 | 0.00 | 0.00 |
| DCT | 0.00 | 0.00 | 0.00 | 0.00 | 0.00 | 3.90 | 0.00 | 5.04 | 0.00 | 0.00 |
| ZBP1 | 0.00 | 0.00 | 7.53 | 0.00 | 0.00 | 0.00 | 0.00 | 5.03 | 0.00 | 0.00 |
| ASXL2 | 0.00 | 0.00 | 0.00 | 0.00 | 0.00 | 0.00 | 0.00 | 5.01 | 0.00 | 0.00 |
| CHD2 | 0.00 | 0.00 | 0.00 | 0.00 | 0.00 | 0.00 | 0.00 | 5.00 | 0.00 | 0.00 |
| S100A8 | 4.13 | 9.04 | 9.99 | 19.14 | 13.64 | 6.35 | 0.00 | 4.97 | 6.68 | 4.72 |
| XRN1 | 0.00 | 0.00 | 0.00 | 0.00 | 0.00 | 0.00 | 0.00 | 4.91 | 0.00 | 0.00 |
| PMS1 | 0.00 | 0.00 | 0.00 | 0.00 | 0.00 | 0.00 | 0.00 | 4.88 | 0.00 | 0.00 |
| GM42418 | 6.04 | 5.10 | 6.63 | 0.00 | 4.06 | 21.47 | 9.19 | 4.72 | 9.29 | 7.63 |
| BTAF1 | 0.00 | 0.00 | 0.00 | 0.00 | 0.00 | 0.00 | 0.00 | 4.72 | 0.00 | 0.00 |
| GM26532 | 0.00 | 0.00 | 3.41 | 0.00 | 0.00 | 5.56 | 0.00 | 4.72 | 0.00 | 0.00 |
| AIF1 | 0.00 | 0.00 | 5.47 | 0.00 | 6.54 | 0.00 | 0.00 | 4.66 | 0.00 | 0.00 |
| VPS13C | 0.00 | 0.00 | 0.00 | 0.00 | 0.00 | 0.00 | 0.00 | 4.50 | 0.00 | 0.00 |
| TFF1 | 0.00 | 0.00 | 0.00 | 0.00 | 7.04 | 0.00 | 0.00 | 4.48 | 0.00 | 0.00 |
| STAT1 | 6.03 | 5.65 | 8.52 | 0.00 | 0.00 | 9.89 | 0.00 | 4.35 | 0.00 | 0.00 |
| AHNAK | 0.00 | 0.00 | 0.00 | 0.00 | 0.00 | 0.00 | 0.00 | 4.34 | 0.00 | 0.00 |
| H2-EB1 | 5.10 | 4.24 | 0.00 | 0.00 | 0.00 | 4.76 | 5.37 | 4.34 | 0.00 | 0.00 |
| CHKA | 0.00 | 0.00 | 0.00 | 0.00 | 0.00 | 0.00 | 0.00 | 4.31 | 0.00 | 0.00 |
| IFIH1 | 0.00 | 0.00 | 0.00 | 0.00 | 0.00 | 0.00 | 0.00 | 4.31 | 0.00 | 0.00 |
| SACS | 0.00 | 0.00 | 0.00 | 0.00 | 7.48 | 0.00 | 0.00 | 4.28 | 0.00 | 0.00 |
| ARMCX5 | 0.00 | 0.00 | 0.00 | 0.00 | 0.00 | 0.00 | 0.00 | 4.23 | 0.00 | 0.00 |
| C1QA | 4.45 | 0.00 | 0.00 | 0.00 | 0.00 | 0.00 | 0.00 | 4.20 | 0.00 | 0.00 |
| LGMN | 0.00 | 0.00 | 0.00 | 0.00 | 0.00 | 0.00 | 0.00 | 4.16 | 0.00 | 0.00 |
| GM4070 | 6.29 | 0.00 | 7.08 | 0.00 | 3.77 | 0.00 | 0.00 | 4.15 | 0.00 | 0.00 |
| SOS1 | 0.00 | 0.00 | 0.00 | 0.00 | 0.00 | 0.00 | 0.00 | 4.11 | 0.00 | 0.00 |
| PMEL | 3.31 | 0.00 | 0.00 | 0.00 | 0.00 | 0.00 | 0.00 | 4.10 | 0.00 | 0.00 |
| PRPF38B | 0.00 | 0.00 | 0.00 | 0.00 | 0.00 | 0.00 | 0.00 | 4.04 | 0.00 | 0.00 |
| ISG15 | 4.85 | 7.33 | 4.90 | 0.00 | 0.00 | 4.88 | 0.00 | 4.03 | 0.00 | 0.00 |
| LNPEP | 0.00 | 0.00 | 0.00 | 0.00 | 3.34 | 0.00 | 0.00 | 4.02 | 0.00 | 0.00 |
| TLN1 | 4.27 | 0.00 | 0.00 | 0.00 | 0.00 | 0.00 | 0.00 | 3.96 | 0.00 | 0.00 |
| FAM189B | 0.00 | 0.00 | 0.00 | 0.00 | 0.00 | 0.00 | 0.00 | 3.95 | 0.00 | 0.00 |
| MLH3 | 0.00 | 0.00 | 0.00 | 0.00 | 0.00 | 0.00 | 0.00 | 3.93 | 0.00 | 0.00 |
| PHF11C | 0.00 | 0.00 | 4.65 | 0.00 | 3.63 | 0.00 | 0.00 | 3.91 | 0.00 | 0.00 |
| SYNRG | 0.00 | 0.00 | 0.00 | 0.00 | 0.00 | 0.00 | 0.00 | 3.88 | 0.00 | 0.00 |
| OAS3 | 0.00 | 0.00 | 3.19 | 0.00 | 0.00 | 0.00 | 0.00 | 3.87 | 0.00 | 0.00 |
| ATP2B1 | 0.00 | 0.00 | 0.00 | 0.00 | 0.00 | 4.14 | 0.00 | 3.85 | 0.00 | 0.00 |
| TLE4 | 0.00 | 0.00 | 0.00 | 0.00 | 0.00 | 0.00 | 0.00 | 3.83 | 0.00 | 0.00 |
| ZFP217 | 0.00 | 0.00 | 0.00 | 0.00 | 0.00 | 0.00 | 0.00 | 3.77 | 0.00 | 0.00 |
| TRAF5 | 0.00 | 0.00 | 0.00 | 0.00 | 0.00 | 0.00 | 0.00 | 3.75 | 0.00 | 0.00 |
| HERPUD1 | 0.00 | 0.00 | 0.00 | 0.00 | 0.00 | 0.00 | 0.00 | 3.75 | 0.00 | 0.00 |
| GM4955 | 0.00 | 0.00 | 5.63 | 0.00 | 0.00 | 0.00 | 0.00 | 3.75 | 0.00 | 0.00 |
| WFDC17 | 3.73 | 0.00 | 0.00 | 0.00 | 0.00 | 0.00 | 0.00 | 3.74 | 0.00 | 0.00 |
| CCNT2 | 0.00 | 0.00 | 0.00 | 0.00 | 0.00 | 0.00 | 0.00 | 3.73 | 0.00 | 0.00 |
| CEP250 | 0.00 | 0.00 | 0.00 | 0.00 | 6.57 | 0.00 | 0.00 | 3.72 | 0.00 | 0.00 |
| MT-ATP8 | 0.00 | 0.00 | 3.68 | 0.00 | 0.00 | 0.00 | 0.00 | 3.72 | 0.00 | 0.00 |
| TNKS2 | 0.00 | 0.00 | 0.00 | 0.00 | 0.00 | 0.00 | 0.00 | 3.69 | 0.00 | 0.00 |
| CNOT1 | 0.00 | 0.00 | 0.00 | 0.00 | 0.00 | 0.00 | 0.00 | 3.69 | 0.00 | 0.00 |
| UBR5 | 0.00 | 0.00 | 0.00 | 0.00 | 0.00 | 0.00 | 0.00 | 3.68 | 0.00 | 0.00 |
| AGAP2 | 0.00 | 0.00 | 0.00 | 0.00 | 0.00 | 0.00 | 0.00 | 3.67 | 0.00 | 0.00 |
| CLEC12A | 0.00 | 0.00 | 0.00 | 0.00 | 0.00 | 0.00 | 0.00 | 3.66 | 0.00 | 0.00 |
| GM16586 | 0.00 | 0.00 | 0.00 | 0.00 | 0.00 | 0.00 | 0.00 | 3.65 | 0.00 | 0.00 |
| PHF14 | 0.00 | 0.00 | 0.00 | 0.00 | 0.00 | 0.00 | 0.00 | 3.65 | 0.00 | 0.00 |
| LYST | 0.00 | 0.00 | 0.00 | 0.00 | 0.00 | 0.00 | 0.00 | 3.65 | 0.00 | 0.00 |
| HIP1 | 0.00 | 0.00 | 0.00 | 0.00 | 0.00 | 0.00 | 0.00 | 3.64 | 0.00 | 0.00 |
| EML4 | 0.00 | 0.00 | 0.00 | 0.00 | 0.00 | 0.00 | 0.00 | 3.63 | 0.00 | 0.00 |
| GVIN1 | 0.00 | 0.00 | 3.15 | 0.00 | 0.00 | 0.00 | 0.00 | 3.62 | 0.00 | 0.00 |
| MADD | 0.00 | 0.00 | 0.00 | 0.00 | 3.92 | 0.00 | 0.00 | 3.61 | 0.00 | 0.00 |
| HOOK1 | 0.00 | 0.00 | 0.00 | 0.00 | 0.00 | 0.00 | 0.00 | 3.60 | 0.00 | 0.00 |
| LYZ2 | 6.98 | 14.43 | 7.33 | 11.33 | 7.72 | 8.30 | 5.22 | 3.59 | 0.00 | 0.00 |
| DPP4 | 0.00 | 0.00 | 0.00 | 0.00 | 0.00 | 0.00 | 0.00 | 3.58 | 0.00 | 0.00 |
| MYB | 0.00 | 0.00 | 0.00 | 0.00 | 0.00 | 0.00 | 0.00 | 3.58 | 0.00 | 0.00 |
| TMEM209 | 0.00 | 0.00 | 0.00 | 0.00 | 0.00 | 0.00 | 0.00 | 3.54 | 0.00 | 0.00 |
| MYO9A | 0.00 | 0.00 | 0.00 | 0.00 | 0.00 | 0.00 | 0.00 | 3.53 | 0.00 | 0.00 |
| SP3 | 0.00 | 0.00 | 0.00 | 0.00 | 0.00 | 0.00 | 0.00 | 3.45 | 0.00 | 0.00 |
| FAM208B | 0.00 | 0.00 | 0.00 | 0.00 | 0.00 | 0.00 | 0.00 | 3.45 | 0.00 | 0.00 |
| TMEM87A | 0.00 | 0.00 | 0.00 | 0.00 | 0.00 | 0.00 | 0.00 | 3.44 | 0.00 | 0.00 |
| PTPN13 | 0.00 | 0.00 | 0.00 | 0.00 | 0.00 | 0.00 | 0.00 | 3.43 | 0.00 | 0.00 |
| CDK13 | 0.00 | 0.00 | 0.00 | 0.00 | 0.00 | 0.00 | 0.00 | 3.42 | 0.00 | 0.00 |
| POLDIP3 | 0.00 | 0.00 | 0.00 | 0.00 | 0.00 | 0.00 | 0.00 | 3.42 | 0.00 | 0.00 |
| C1QC | 0.00 | 0.00 | 0.00 | 0.00 | 0.00 | 0.00 | 0.00 | 3.41 | 0.00 | 0.00 |
| MBD5 | 0.00 | 0.00 | 0.00 | 0.00 | 0.00 | 0.00 | 0.00 | 3.41 | 0.00 | 0.00 |
| IFIT1BL1 | 0.00 | 0.00 | 0.00 | 0.00 | 0.00 | 4.33 | 0.00 | 3.41 | 0.00 | 0.00 |
| ITPR2 | 0.00 | 0.00 | 0.00 | 0.00 | 0.00 | 0.00 | 0.00 | 3.40 | 0.00 | 0.00 |
| ZFP874B | 0.00 | 0.00 | 0.00 | 0.00 | 0.00 | 0.00 | 0.00 | 3.40 | 0.00 | 0.00 |
| SSH1 | 0.00 | 0.00 | 0.00 | 0.00 | 0.00 | 0.00 | 0.00 | 3.40 | 0.00 | 0.00 |
| HBA-A1 | 0.00 | 0.00 | 4.64 | 0.00 | 0.00 | 0.00 | 0.00 | 3.38 | 0.00 | 4.13 |
| STRIP1 | 0.00 | 0.00 | 0.00 | 0.00 | 0.00 | 0.00 | 0.00 | 3.36 | 0.00 | 0.00 |
| SLMAP | 0.00 | 0.00 | 0.00 | 0.00 | 0.00 | 0.00 | 0.00 | 3.36 | 0.00 | 0.00 |
| 1700020I14RIK | 0.00 | 0.00 | 0.00 | 0.00 | 3.29 | 0.00 | 0.00 | 0.00 | 0.00 | 0.00 |
| 2310040G24RIK | 0.00 | 0.00 | 0.00 | 0.00 | 0.00 | 0.00 | 0.00 | 0.00 | 0.00 | 0.00 |
| 2510046G10RIK | 0.00 | 0.00 | 0.00 | 0.00 | 0.00 | 0.00 | 0.00 | 0.00 | 0.00 | 0.00 |
| 2810474O19RIK | 0.00 | 0.00 | 0.00 | 0.00 | 0.00 | 0.00 | 0.00 | 0.00 | 0.00 | 0.00 |
| 2900060B14RIK | 0.00 | 0.00 | 0.00 | 0.00 | 0.00 | 0.00 | 0.00 | 0.00 | 0.00 | 0.00 |
| 4932438A13RIK | 0.00 | 0.00 | 0.00 | 0.00 | 3.85 | 0.00 | 0.00 | 0.00 | 0.00 | 0.00 |
| 9930111J21RIK1 | 0.00 | 0.00 | 0.00 | 0.00 | 0.00 | 0.00 | 0.00 | 0.00 | 0.00 | 0.00 |
| 9930111J21RIK2 | 0.00 | 4.59 | 5.44 | 0.00 | 0.00 | 0.00 | 0.00 | 0.00 | 0.00 | 0.00 |
| A530040E14RIK | 0.00 | 0.00 | 0.00 | 0.00 | 0.00 | 0.00 | 0.00 | 0.00 | 0.00 | 0.00 |
| A930007I19RIK | 0.00 | 0.00 | 0.00 | 0.00 | 0.00 | 0.00 | 0.00 | 0.00 | 0.00 | 0.00 |
| AA467197 | 0.00 | 0.00 | 3.32 | 0.00 | 0.00 | 0.00 | 0.00 | 0.00 | 0.00 | 0.00 |
| ABLIM1 | 0.00 | 0.00 | 0.00 | 0.00 | 0.00 | 0.00 | 0.00 | 0.00 | 0.00 | 0.00 |
| ACTB | 0.00 | 0.00 | 0.00 | 0.00 | 0.00 | 0.00 | 0.00 | 0.00 | 0.00 | 0.00 |
| ACTG1 | 0.00 | 0.00 | 4.44 | 0.00 | 0.00 | 0.00 | 0.00 | 0.00 | 0.00 | 0.00 |
| ACTR3 | 0.00 | 0.00 | 0.00 | 0.00 | 0.00 | 0.00 | 0.00 | 0.00 | 0.00 | 0.00 |
| ADAM8 | 0.00 | 0.00 | 0.00 | 0.00 | 0.00 | 0.00 | 0.00 | 0.00 | 0.00 | 0.00 |
| ADAP2 | 0.00 | 0.00 | 0.00 | 0.00 | 0.00 | 0.00 | 0.00 | 0.00 | 0.00 | 0.00 |
| ADK | 3.39 | 0.00 | 0.00 | 0.00 | 0.00 | 0.00 | 0.00 | 0.00 | 0.00 | 0.00 |
| ADPGK | 0.00 | 0.00 | 0.00 | 0.00 | 0.00 | 0.00 | 0.00 | 0.00 | 0.00 | 0.00 |
| ADRB2 | 3.56 | 0.00 | 0.00 | 0.00 | 0.00 | 0.00 | 0.00 | 0.00 | 0.00 | 0.00 |
| ADRBK2 | 0.00 | 0.00 | 0.00 | 0.00 | 0.00 | 0.00 | 0.00 | 0.00 | 0.00 | 0.00 |
| AFF1 | 0.00 | 0.00 | 0.00 | 0.00 | 0.00 | 0.00 | 0.00 | 0.00 | 0.00 | 0.00 |
| AFG3L2 | 0.00 | 0.00 | 0.00 | 0.00 | 0.00 | 0.00 | 0.00 | 0.00 | 0.00 | 0.00 |
| AHCYL2 | 0.00 | 0.00 | 0.00 | 0.00 | 4.37 | 0.00 | 0.00 | 0.00 | 0.00 | 0.00 |
| AI607873 | 0.00 | 0.00 | 0.00 | 0.00 | 0.00 | 0.00 | 0.00 | 0.00 | 0.00 | 0.00 |
| AKAP10 | 0.00 | 0.00 | 0.00 | 0.00 | 3.45 | 0.00 | 0.00 | 0.00 | 0.00 | 0.00 |
| AKAP11 | 0.00 | 0.00 | 0.00 | 0.00 | 3.38 | 0.00 | 0.00 | 0.00 | 0.00 | 0.00 |
| ANKRD33B | 0.00 | 0.00 | 0.00 | 0.00 | 0.00 | 0.00 | 0.00 | 0.00 | 0.00 | 0.00 |
| ANXA1 | 0.00 | 0.00 | 0.00 | 0.00 | 0.00 | 0.00 | 0.00 | 0.00 | 0.00 | 0.00 |
| ANXA2 | 0.00 | 0.00 | 0.00 | 3.77 | 0.00 | 0.00 | 0.00 | 0.00 | 0.00 | 0.00 |
| AOAH | 0.00 | 0.00 | 0.00 | 0.00 | 0.00 | 0.00 | 0.00 | 0.00 | 0.00 | 0.00 |
| AP2B1 | 0.00 | 0.00 | 0.00 | 0.00 | 3.45 | 0.00 | 0.00 | 0.00 | 0.00 | 0.00 |
| AP3M2 | 0.00 | 0.00 | 0.00 | 0.00 | 0.00 | 0.00 | 0.00 | 0.00 | 0.00 | 0.00 |
| APC | 0.00 | 0.00 | 0.00 | 0.00 | 3.59 | 0.00 | 0.00 | 0.00 | 0.00 | 0.00 |
| APOBR | 0.00 | 0.00 | 0.00 | 0.00 | 3.28 | 0.00 | 0.00 | 0.00 | 0.00 | 0.00 |
| APOD | 0.00 | 0.00 | 0.00 | 0.00 | 0.00 | 0.00 | 0.00 | 0.00 | 0.00 | 0.00 |
| APOE | 0.00 | 0.00 | 0.00 | 0.00 | 0.00 | 0.00 | 0.00 | 0.00 | 0.00 | 0.00 |
| APOL9B | 0.00 | 0.00 | 0.00 | 0.00 | 0.00 | 4.18 | 0.00 | 0.00 | 0.00 | 0.00 |
| ARG2 | 5.75 | 0.00 | 0.00 | 0.00 | 0.00 | 0.00 | 0.00 | 0.00 | 0.00 | 0.00 |
| ARL5A | 0.00 | 0.00 | 0.00 | 0.00 | 0.00 | 4.10 | 0.00 | 0.00 | 0.00 | 0.00 |
| ARMC7 | 0.00 | 0.00 | 0.00 | 0.00 | 0.00 | 0.00 | 0.00 | 0.00 | 0.00 | 0.00 |
| ASB13 | 0.00 | 0.00 | 0.00 | 0.00 | 0.00 | 0.00 | 0.00 | 0.00 | 0.00 | 0.00 |
| ASS1 | 0.00 | 0.00 | 0.00 | 0.00 | 3.82 | 0.00 | 0.00 | 0.00 | 0.00 | 0.00 |
| ASXL1 | 0.00 | 0.00 | 0.00 | 0.00 | 5.42 | 0.00 | 0.00 | 0.00 | 0.00 | 0.00 |
| ATF6 | 0.00 | 0.00 | 0.00 | 0.00 | 3.41 | 0.00 | 0.00 | 0.00 | 0.00 | 0.00 |
| ATF7IP | 3.76 | 0.00 | 0.00 | 0.00 | 0.00 | 0.00 | 0.00 | 0.00 | 0.00 | 0.00 |
| ATP11B | 4.67 | 0.00 | 0.00 | 0.00 | 0.00 | 0.00 | 0.00 | 0.00 | 0.00 | 0.00 |
| ATP8B4 | 0.00 | 0.00 | 0.00 | 0.00 | 6.83 | 0.00 | 0.00 | 0.00 | 0.00 | 0.00 |
| AU020206 | 0.00 | 0.00 | 0.00 | 0.00 | 3.63 | 0.00 | 0.00 | 0.00 | 0.00 | 0.00 |
| AW112010 | 4.20 | 0.00 | 0.00 | 0.00 | 0.00 | 0.00 | 0.00 | 0.00 | 0.00 | 0.00 |
| AY036118 | 4.85 | 5.28 | 6.34 | 0.00 | 0.00 | 6.33 | 6.31 | 0.00 | 0.00 | 0.00 |
| B130006D01RIK | 0.00 | 0.00 | 0.00 | 0.00 | 0.00 | 0.00 | 0.00 | 0.00 | 0.00 | 0.00 |
| B2M | 0.00 | 0.00 | 4.02 | 0.00 | 0.00 | 0.00 | 0.00 | 0.00 | 0.00 | 0.00 |
| B430306N03RIK | 0.00 | 0.00 | 0.00 | 0.00 | 0.00 | 0.00 | 0.00 | 0.00 | 0.00 | 0.00 |
| B4GALT1 | 0.00 | 0.00 | 0.00 | 0.00 | 0.00 | 0.00 | 0.00 | 0.00 | 0.00 | 0.00 |
| B4GALT4 | 0.00 | 0.00 | 0.00 | 0.00 | 0.00 | 0.00 | 0.00 | 0.00 | 0.00 | 0.00 |
| BACH2 | 0.00 | 0.00 | 3.38 | 0.00 | 0.00 | 0.00 | 0.00 | 0.00 | 0.00 | 0.00 |
| BASP1 | 0.00 | 0.00 | 0.00 | 0.00 | 0.00 | 0.00 | 0.00 | 0.00 | 0.00 | 0.00 |
| BATF2 | 0.00 | 0.00 | 0.00 | 0.00 | 0.00 | 0.00 | 0.00 | 0.00 | 0.00 | 0.00 |
| BATF3 | 0.00 | 0.00 | 0.00 | 0.00 | 0.00 | 0.00 | 0.00 | 0.00 | 0.00 | 0.00 |
| BAZ1A | 0.00 | 0.00 | 3.34 | 0.00 | 0.00 | 0.00 | 0.00 | 0.00 | 0.00 | 0.00 |
| BAZ2B | 0.00 | 0.00 | 0.00 | 0.00 | 3.45 | 0.00 | 0.00 | 0.00 | 0.00 | 0.00 |
| BBC3 | 3.61 | 0.00 | 0.00 | 0.00 | 0.00 | 0.00 | 0.00 | 0.00 | 0.00 | 0.00 |
| BC094916 | 0.00 | 0.00 | 4.47 | 0.00 | 0.00 | 0.00 | 0.00 | 0.00 | 0.00 | 0.00 |
| BC147527 | 0.00 | 0.00 | 0.00 | 0.00 | 0.00 | 0.00 | 0.00 | 0.00 | 0.00 | 0.00 |
| BCL2L2 | 0.00 | 0.00 | 0.00 | 0.00 | 0.00 | 0.00 | 0.00 | 0.00 | 0.00 | 0.00 |
| BEX6 | 0.00 | 0.00 | 0.00 | 0.00 | 0.00 | 0.00 | 0.00 | 0.00 | 0.00 | 0.00 |
| BLVRB | 0.00 | 0.00 | 0.00 | 0.00 | 0.00 | 0.00 | 0.00 | 0.00 | 0.00 | 0.00 |
| BMYC | 0.00 | 0.00 | 3.78 | 0.00 | 0.00 | 0.00 | 0.00 | 0.00 | 0.00 | 0.00 |
| BNIP3 | 0.00 | 0.00 | 4.67 | 0.00 | 0.00 | 0.00 | 0.00 | 0.00 | 0.00 | 0.00 |
| BNIP3L | 0.00 | 0.00 | 0.00 | 0.00 | 0.00 | 0.00 | 0.00 | 0.00 | 0.00 | 0.00 |
| BRD3 | 0.00 | 0.00 | 0.00 | 0.00 | 0.00 | 0.00 | 0.00 | 0.00 | 0.00 | 0.00 |
| BST1 | 0.00 | 0.00 | 0.00 | 0.00 | 0.00 | 0.00 | 0.00 | 0.00 | 0.00 | 0.00 |
| BST2 | 0.00 | 0.00 | 5.60 | 0.00 | 0.00 | 0.00 | 0.00 | 0.00 | 0.00 | 0.00 |
| BTG2 | 0.00 | 0.00 | 0.00 | 0.00 | 0.00 | 0.00 | 0.00 | 0.00 | 0.00 | 0.00 |
| C130026I21RIK | 0.00 | 0.00 | 0.00 | 0.00 | 0.00 | 0.00 | 0.00 | 0.00 | 0.00 | 0.00 |
| C5AR1 | 0.00 | 0.00 | 0.00 | 0.00 | 0.00 | 0.00 | 0.00 | 0.00 | 0.00 | 0.00 |
| CALM1 | 0.00 | 0.00 | 0.00 | 0.00 | 0.00 | 0.00 | 0.00 | 0.00 | 0.00 | 0.00 |
| CAPNS1 | 0.00 | 0.00 | 3.19 | 0.00 | 0.00 | 0.00 | 0.00 | 0.00 | 0.00 | 0.00 |
| CAR14 | 0.00 | 0.00 | 0.00 | 0.00 | 3.82 | 0.00 | 0.00 | 0.00 | 0.00 | 0.00 |
| CARD19 | 0.00 | 0.00 | 0.00 | 0.00 | 0.00 | 0.00 | 0.00 | 0.00 | 0.00 | 0.00 |
| CASC5 | 0.00 | 0.00 | 0.00 | 0.00 | 0.00 | 0.00 | 0.00 | 0.00 | 6.27 | 0.00 |
| CASP4 | 0.00 | 0.00 | 0.00 | 0.00 | 0.00 | 0.00 | 0.00 | 0.00 | 0.00 | 0.00 |
| CASP8AP2 | 0.00 | 0.00 | 4.31 | 0.00 | 0.00 | 0.00 | 0.00 | 0.00 | 0.00 | 0.00 |
| CBX5 | 0.00 | 0.00 | 0.00 | 0.00 | 4.35 | 0.00 | 0.00 | 0.00 | 0.00 | 0.00 |
| CCDC141 | 0.00 | 0.00 | 0.00 | 4.59 | 0.00 | 0.00 | 0.00 | 0.00 | 0.00 | 0.00 |
| CCDC162 | 3.38 | 0.00 | 0.00 | 0.00 | 0.00 | 0.00 | 0.00 | 0.00 | 0.00 | 0.00 |
| CCDC88A | 0.00 | 0.00 | 0.00 | 0.00 | 0.00 | 0.00 | 0.00 | 0.00 | 0.00 | 0.00 |
| CCDC88C | 0.00 | 0.00 | 0.00 | 0.00 | 4.21 | 0.00 | 0.00 | 0.00 | 0.00 | 0.00 |
| CCDC94 | 0.00 | 0.00 | 0.00 | 0.00 | 0.00 | 0.00 | 0.00 | 0.00 | 0.00 | 0.00 |
| CCL1 | 0.00 | 0.00 | 0.00 | 0.00 | 0.00 | 0.00 | 0.00 | 0.00 | 0.00 | 0.00 |
| CCL12 | 0.00 | 0.00 | 0.00 | 0.00 | 0.00 | 0.00 | 0.00 | 0.00 | 0.00 | 0.00 |
| CCL2 | 0.00 | 0.00 | 0.00 | 4.08 | 0.00 | 0.00 | 0.00 | 0.00 | 0.00 | 0.00 |
| CCL24 | 0.00 | 0.00 | 0.00 | 0.00 | 0.00 | 0.00 | 0.00 | 0.00 | 0.00 | 0.00 |
| CCL3 | 12.80 | 0.00 | 0.00 | 0.00 | 0.00 | 0.00 | 0.00 | 0.00 | 0.00 | 0.00 |
| CCL4 | 30.70 | 0.00 | 0.00 | 0.00 | 0.00 | 0.00 | 0.00 | 0.00 | 0.00 | 0.00 |
| CCL5 | 3.53 | 0.00 | 0.00 | 0.00 | 0.00 | 8.46 | 0.00 | 0.00 | 0.00 | 0.00 |
| CCL7 | 0.00 | 0.00 | 4.10 | 0.00 | 0.00 | 0.00 | 4.53 | 0.00 | 0.00 | 0.00 |
| CCL9 | 0.00 | 0.00 | 0.00 | 0.00 | 0.00 | 0.00 | 0.00 | 0.00 | 0.00 | 0.00 |
| CCNB1 | 0.00 | 0.00 | 0.00 | 0.00 | 0.00 | 0.00 | 0.00 | 0.00 | 0.00 | 0.00 |
| CCND1 | 0.00 | 0.00 | 0.00 | 0.00 | 0.00 | 0.00 | 0.00 | 0.00 | 0.00 | 0.00 |
| CCND2 | 0.00 | 0.00 | 0.00 | 0.00 | 0.00 | 0.00 | 0.00 | 0.00 | 0.00 | 0.00 |
| CCNE2 | 0.00 | 0.00 | 0.00 | 0.00 | 0.00 | 0.00 | 0.00 | 0.00 | 0.00 | 0.00 |
| CCNG2 | 0.00 | 0.00 | 0.00 | 0.00 | 0.00 | 0.00 | 0.00 | 0.00 | 0.00 | 0.00 |
| CCNL1 | 0.00 | 0.00 | 0.00 | 0.00 | 0.00 | 0.00 | 0.00 | 0.00 | 0.00 | 0.00 |
| CCR1 | 0.00 | 0.00 | 0.00 | 0.00 | 0.00 | 0.00 | 0.00 | 0.00 | 0.00 | 0.00 |
| CCR2 | 0.00 | 0.00 | 0.00 | 0.00 | 0.00 | 0.00 | 0.00 | 0.00 | 0.00 | 0.00 |
| CCR3 | 0.00 | 0.00 | 5.46 | 0.00 | 0.00 | 0.00 | 0.00 | 0.00 | 0.00 | 0.00 |
| CCR7 | 0.00 | 0.00 | 4.29 | 0.00 | 0.00 | 0.00 | 0.00 | 0.00 | 0.00 | 0.00 |
| CCRL2 | 0.00 | 0.00 | 0.00 | 0.00 | 0.00 | 0.00 | 0.00 | 0.00 | 0.00 | 0.00 |
| CD14 | 0.00 | 0.00 | 0.00 | 0.00 | 0.00 | 0.00 | 0.00 | 0.00 | 0.00 | 0.00 |
| CD160 | 4.68 | 0.00 | 0.00 | 0.00 | 0.00 | 0.00 | 0.00 | 0.00 | 0.00 | 0.00 |
| CD177 | 0.00 | 0.00 | 0.00 | 0.00 | 0.00 | 0.00 | 0.00 | 0.00 | 0.00 | 0.00 |
| CD180 | 0.00 | 0.00 | 0.00 | 0.00 | 0.00 | 0.00 | 0.00 | 0.00 | 0.00 | 0.00 |
| CD2 | 0.00 | 0.00 | 0.00 | 0.00 | 0.00 | 0.00 | 0.00 | 0.00 | 0.00 | 0.00 |
| CD200 | 0.00 | 0.00 | 4.82 | 0.00 | 0.00 | 0.00 | 0.00 | 0.00 | 0.00 | 0.00 |
| CD209A | 0.00 | 0.00 | 0.00 | 0.00 | 0.00 | 0.00 | 0.00 | 0.00 | 0.00 | 0.00 |
| CD226 | 0.00 | 0.00 | 0.00 | 0.00 | 0.00 | 0.00 | 0.00 | 0.00 | 0.00 | 0.00 |
| CD24A | 0.00 | 0.00 | 0.00 | 0.00 | 0.00 | 0.00 | 0.00 | 0.00 | 0.00 | 0.00 |
| CD274 | 0.00 | 0.00 | 3.55 | 0.00 | 0.00 | 0.00 | 0.00 | 0.00 | 0.00 | 0.00 |
| CD28 | 5.07 | 0.00 | 0.00 | 0.00 | 0.00 | 0.00 | 0.00 | 0.00 | 0.00 | 0.00 |
| CD2AP | 0.00 | 0.00 | 0.00 | 0.00 | 0.00 | 0.00 | 0.00 | 0.00 | 0.00 | 0.00 |
| CD300LD | 0.00 | 0.00 | 0.00 | 0.00 | 0.00 | 0.00 | 0.00 | 0.00 | 0.00 | 0.00 |
| CD300LF | 0.00 | 0.00 | 0.00 | 0.00 | 0.00 | 0.00 | 0.00 | 0.00 | 0.00 | 0.00 |
| CD4 | 0.00 | 0.00 | 0.00 | 0.00 | 0.00 | 0.00 | 0.00 | 0.00 | 0.00 | 4.93 |
| CD40 | 0.00 | 0.00 | 0.00 | 0.00 | 0.00 | 0.00 | 0.00 | 0.00 | 0.00 | 0.00 |
| CD52 | 4.59 | 0.00 | 0.00 | 0.00 | 0.00 | 0.00 | 0.00 | 0.00 | 0.00 | 0.00 |
| CD55 | 0.00 | 0.00 | 0.00 | 0.00 | 0.00 | 0.00 | 0.00 | 0.00 | 0.00 | 0.00 |
| CD63 | 0.00 | 0.00 | 0.00 | 0.00 | 0.00 | 0.00 | 0.00 | 0.00 | 0.00 | 0.00 |
| CD74 | 0.00 | 4.06 | 0.00 | 0.00 | 0.00 | 4.60 | 4.84 | 0.00 | 0.00 | 0.00 |
| CD79A | 0.00 | 0.00 | 0.00 | 0.00 | 0.00 | 0.00 | 0.00 | 0.00 | 0.00 | 0.00 |
| CD79B | 0.00 | 0.00 | 4.10 | 0.00 | 0.00 | 0.00 | 0.00 | 0.00 | 0.00 | 0.00 |
| CD81 | 0.00 | 4.11 | 4.80 | 0.00 | 0.00 | 0.00 | 0.00 | 0.00 | 0.00 | 0.00 |
| CD83 | 0.00 | 0.00 | 0.00 | 0.00 | 0.00 | 0.00 | 0.00 | 0.00 | 0.00 | 0.00 |
| CD86 | 3.73 | 0.00 | 0.00 | 0.00 | 0.00 | 0.00 | 0.00 | 0.00 | 0.00 | 0.00 |
| CD9 | 0.00 | 0.00 | 0.00 | 0.00 | 0.00 | 0.00 | 0.00 | 0.00 | 0.00 | 0.00 |
| CDC14A | 0.00 | 0.00 | 0.00 | 0.00 | 3.74 | 0.00 | 0.00 | 0.00 | 0.00 | 0.00 |
| CDKN2C | 0.00 | 0.00 | 0.00 | 0.00 | 0.00 | 0.00 | 0.00 | 0.00 | 0.00 | 0.00 |
| CEACAM1 | 0.00 | 0.00 | 0.00 | 0.00 | 0.00 | 0.00 | 0.00 | 0.00 | 0.00 | 0.00 |
| CEBPB | 6.43 | 0.00 | 0.00 | 0.00 | 0.00 | 0.00 | 0.00 | 0.00 | 0.00 | 0.00 |
| CENPH | 0.00 | 0.00 | 0.00 | 0.00 | 0.00 | 0.00 | 0.00 | 0.00 | 0.00 | 0.00 |
| CEP170 | 0.00 | 0.00 | 0.00 | 0.00 | 3.73 | 0.00 | 0.00 | 0.00 | 0.00 | 0.00 |
| CEP350 | 0.00 | 0.00 | 0.00 | 0.00 | 0.00 | 0.00 | 0.00 | 0.00 | 0.00 | 0.00 |
| CERS4 | 0.00 | 0.00 | 0.00 | 0.00 | 3.30 | 0.00 | 0.00 | 0.00 | 0.00 | 0.00 |
| CFB | 0.00 | 0.00 | 0.00 | 0.00 | 0.00 | 0.00 | 0.00 | 0.00 | 0.00 | 0.00 |
| CFH | 0.00 | 0.00 | 0.00 | 0.00 | 0.00 | 0.00 | 0.00 | 0.00 | 0.00 | 0.00 |
| CHURC1 | 0.00 | 0.00 | 0.00 | 3.93 | 0.00 | 0.00 | 0.00 | 0.00 | 0.00 | 0.00 |
| CKB | 0.00 | 0.00 | 0.00 | 0.00 | 0.00 | 0.00 | 0.00 | 0.00 | 0.00 | 0.00 |
| CLDND1 | 0.00 | 0.00 | 0.00 | 0.00 | 0.00 | 0.00 | 0.00 | 0.00 | 0.00 | 0.00 |
| CLEC2D | 3.56 | 0.00 | 6.30 | 0.00 | 0.00 | 3.90 | 0.00 | 0.00 | 0.00 | 0.00 |
| CLEC4D | 0.00 | 0.00 | 0.00 | 0.00 | 0.00 | 0.00 | 0.00 | 0.00 | 0.00 | 0.00 |
| CLEC4E | 0.00 | 0.00 | 0.00 | 0.00 | 0.00 | 0.00 | 0.00 | 0.00 | 0.00 | 0.00 |
| CLEC4N | 0.00 | 0.00 | 0.00 | 0.00 | 0.00 | 0.00 | 0.00 | 0.00 | 0.00 | 0.00 |
| CLIC4 | 0.00 | 0.00 | 0.00 | 0.00 | 0.00 | 0.00 | 0.00 | 0.00 | 0.00 | 0.00 |
| CLK1 | 4.79 | 0.00 | 0.00 | 0.00 | 0.00 | 0.00 | 0.00 | 0.00 | 0.00 | 0.00 |
| CMPK2 | 0.00 | 0.00 | 0.00 | 0.00 | 0.00 | 0.00 | 0.00 | 0.00 | 0.00 | 0.00 |
| CMTR1 | 3.67 | 0.00 | 0.00 | 0.00 | 0.00 | 0.00 | 0.00 | 0.00 | 0.00 | 0.00 |
| CNP | 0.00 | 0.00 | 0.00 | 0.00 | 0.00 | 0.00 | 0.00 | 0.00 | 0.00 | 0.00 |
| COL4A3BP | 0.00 | 0.00 | 7.34 | 0.00 | 0.00 | 0.00 | 0.00 | 0.00 | 0.00 | 0.00 |
| COX6A2 | 0.00 | 0.00 | 0.00 | 0.00 | 0.00 | 0.00 | 0.00 | 0.00 | 7.79 | 0.00 |
| CPSF3L | 0.00 | 0.00 | 0.00 | 0.00 | 3.34 | 0.00 | 0.00 | 0.00 | 0.00 | 0.00 |
| CRIP1 | 0.00 | 0.00 | 3.27 | 0.00 | 0.00 | 0.00 | 0.00 | 0.00 | 0.00 | 0.00 |
| CRLF3 | 3.38 | 0.00 | 0.00 | 0.00 | 0.00 | 0.00 | 0.00 | 0.00 | 0.00 | 0.00 |
| CRYBB1 | 0.00 | 0.00 | 0.00 | 0.00 | 0.00 | 0.00 | 0.00 | 0.00 | 0.00 | 0.00 |
| CSRNP1 | 0.00 | 0.00 | 0.00 | 0.00 | 0.00 | 0.00 | 0.00 | 0.00 | 0.00 | 0.00 |
| CSTB | 0.00 | 0.00 | 0.00 | 0.00 | 0.00 | 0.00 | 0.00 | 0.00 | 0.00 | 0.00 |
| CTLA2A | 3.43 | 0.00 | 0.00 | 0.00 | 0.00 | 0.00 | 0.00 | 0.00 | 0.00 | 0.00 |
| CTLA4 | 0.00 | 0.00 | 0.00 | 0.00 | 0.00 | 0.00 | 0.00 | 0.00 | 0.00 | 10.44 |
| CTSS | 0.00 | 0.00 | 5.23 | 0.00 | 0.00 | 4.43 | 0.00 | 0.00 | 0.00 | 0.00 |
| CTSW | 4.61 | 0.00 | 0.00 | 0.00 | 0.00 | 0.00 | 0.00 | 0.00 | 0.00 | 0.00 |
| CXCL10 | 0.00 | 0.00 | 3.89 | 0.00 | 0.00 | 5.89 | 0.00 | 0.00 | 0.00 | 0.00 |
| CXCL2 | 22.27 | 0.00 | 0.00 | 5.61 | 0.00 | 0.00 | 0.00 | 0.00 | 0.00 | 0.00 |
| CXCL9 | 0.00 | 0.00 | 0.00 | 0.00 | 0.00 | 0.00 | 0.00 | 0.00 | 0.00 | 0.00 |
| CXCR3 | 4.34 | 0.00 | 0.00 | 0.00 | 0.00 | 0.00 | 0.00 | 0.00 | 0.00 | 0.00 |
| CXCR4 | 0.00 | 0.00 | 3.33 | 0.00 | 0.00 | 0.00 | 0.00 | 0.00 | 0.00 | 0.00 |
| CYBB | 0.00 | 0.00 | 0.00 | 0.00 | 0.00 | 0.00 | 0.00 | 0.00 | 6.09 | 0.00 |
| CYCS | 0.00 | 0.00 | 0.00 | 0.00 | 0.00 | 0.00 | 0.00 | 0.00 | 0.00 | 0.00 |
| CYFIP1 | 0.00 | 0.00 | 0.00 | 0.00 | 0.00 | 0.00 | 0.00 | 0.00 | 0.00 | 0.00 |
| CYTH4 | 0.00 | 0.00 | 0.00 | 0.00 | 0.00 | 0.00 | 0.00 | 0.00 | 0.00 | 0.00 |
| D030056L22RIK | 0.00 | 0.00 | 0.00 | 0.00 | 3.51 | 0.00 | 0.00 | 0.00 | 0.00 | 0.00 |
| D13ERTD608E | 0.00 | 0.00 | 3.64 | 0.00 | 0.00 | 0.00 | 0.00 | 0.00 | 0.00 | 0.00 |
| D17WSU92E | 0.00 | 0.00 | 0.00 | 0.00 | 0.00 | 0.00 | 0.00 | 0.00 | 0.00 | 0.00 |
| D1ERTD622E | 3.74 | 0.00 | 0.00 | 0.00 | 0.00 | 0.00 | 0.00 | 0.00 | 0.00 | 0.00 |
| D930015E06RIK | 0.00 | 0.00 | 0.00 | 0.00 | 3.92 | 0.00 | 0.00 | 0.00 | 0.00 | 0.00 |
| DAXX | 0.00 | 0.00 | 0.00 | 0.00 | 0.00 | 0.00 | 0.00 | 0.00 | 0.00 | 0.00 |
| DCP1B | 0.00 | 0.00 | 0.00 | 0.00 | 0.00 | 0.00 | 0.00 | 0.00 | 0.00 | 0.00 |
| DDIT3 | 0.00 | 0.00 | 0.00 | 0.00 | 0.00 | 0.00 | 0.00 | 0.00 | 0.00 | 0.00 |
| DDIT4 | 0.00 | 0.00 | 0.00 | 4.44 | 0.00 | 0.00 | 0.00 | 0.00 | 0.00 | 0.00 |
| DDX46 | 0.00 | 0.00 | 3.24 | 0.00 | 0.00 | 0.00 | 0.00 | 0.00 | 0.00 | 0.00 |
| DDX58 | 5.08 | 0.00 | 0.00 | 0.00 | 0.00 | 0.00 | 0.00 | 0.00 | 0.00 | 0.00 |
| DEDD2 | 0.00 | 0.00 | 0.00 | 0.00 | 0.00 | 0.00 | 0.00 | 0.00 | 0.00 | 0.00 |
| DENND1B | 0.00 | 0.00 | 0.00 | 0.00 | 4.68 | 0.00 | 0.00 | 0.00 | 0.00 | 0.00 |
| DGAT1 | 0.00 | 0.00 | 0.00 | 0.00 | 0.00 | 0.00 | 0.00 | 0.00 | 0.00 | 4.48 |
| DGAT2 | 0.00 | 0.00 | 0.00 | 0.00 | 0.00 | 0.00 | 0.00 | 0.00 | 0.00 | 0.00 |
| DGCR8 | 0.00 | 0.00 | 3.76 | 0.00 | 0.00 | 0.00 | 0.00 | 0.00 | 0.00 | 0.00 |
| DHX29 | 0.00 | 0.00 | 0.00 | 0.00 | 6.10 | 0.00 | 0.00 | 0.00 | 0.00 | 0.00 |
| DHX58 | 0.00 | 0.00 | 0.00 | 0.00 | 0.00 | 0.00 | 0.00 | 0.00 | 0.00 | 0.00 |
| DHX9 | 0.00 | 0.00 | 0.00 | 0.00 | 0.00 | 0.00 | 0.00 | 0.00 | 0.00 | 0.00 |
| DIAPH1 | 0.00 | 0.00 | 0.00 | 0.00 | 0.00 | 0.00 | 0.00 | 0.00 | 0.00 | 0.00 |
| DIP2B | 0.00 | 0.00 | 0.00 | 0.00 | 3.95 | 0.00 | 0.00 | 0.00 | 0.00 | 0.00 |
| DKKL1 | 0.00 | 0.00 | 0.00 | 0.00 | 3.58 | 0.00 | 0.00 | 0.00 | 0.00 | 0.00 |
| DLG1 | 0.00 | 0.00 | 0.00 | 0.00 | 4.12 | 0.00 | 0.00 | 0.00 | 7.70 | 0.00 |
| DNAJB1 | 0.00 | 0.00 | 4.14 | 6.41 | 0.00 | 0.00 | 0.00 | 0.00 | 0.00 | 0.00 |
| DNAJC13 | 0.00 | 0.00 | 0.00 | 0.00 | 3.63 | 0.00 | 0.00 | 0.00 | 0.00 | 0.00 |
| DOPEY2 | 0.00 | 0.00 | 0.00 | 0.00 | 0.00 | 0.00 | 0.00 | 0.00 | 0.00 | 0.00 |
| DTX3L | 0.00 | 0.00 | 0.00 | 0.00 | 3.38 | 0.00 | 0.00 | 0.00 | 0.00 | 0.00 |
| DUSP1 | 0.00 | 0.00 | 0.00 | 0.00 | 0.00 | 3.61 | 0.00 | 0.00 | 0.00 | 0.00 |
| DUSP2 | 5.52 | 0.00 | 0.00 | 3.84 | 0.00 | 0.00 | 0.00 | 0.00 | 0.00 | 0.00 |
| DUSP7 | 0.00 | 0.00 | 0.00 | 0.00 | 0.00 | 0.00 | 0.00 | 0.00 | 0.00 | 0.00 |
| E130311K13RIK | 0.00 | 0.00 | 0.00 | 0.00 | 0.00 | 0.00 | 0.00 | 0.00 | 0.00 | 0.00 |
| EBF1 | 0.00 | 0.00 | 6.57 | 0.00 | 0.00 | 0.00 | 0.00 | 0.00 | 0.00 | 0.00 |
| ECE1 | 0.00 | 0.00 | 0.00 | 0.00 | 0.00 | 0.00 | 0.00 | 0.00 | 0.00 | 0.00 |
| EEA1 | 0.00 | 0.00 | 3.19 | 0.00 | 0.00 | 0.00 | 0.00 | 0.00 | 0.00 | 0.00 |
| EED | 0.00 | 0.00 | 0.00 | 0.00 | 3.33 | 0.00 | 0.00 | 0.00 | 0.00 | 0.00 |
| EEF1A1 | 0.00 | 0.00 | 0.00 | 0.00 | 0.00 | 4.39 | 0.00 | 0.00 | 0.00 | 0.00 |
| EGLN3 | 0.00 | 0.00 | 0.00 | 0.00 | 0.00 | 0.00 | 0.00 | 0.00 | 0.00 | 0.00 |
| EGR1 | 0.00 | 0.00 | 0.00 | 0.00 | 0.00 | 0.00 | 0.00 | 0.00 | 0.00 | 0.00 |
| EIF2AK2 | 0.00 | 0.00 | 0.00 | 0.00 | 0.00 | 0.00 | 0.00 | 0.00 | 0.00 | 0.00 |
| EIF4E3 | 0.00 | 0.00 | 0.00 | 0.00 | 3.42 | 0.00 | 0.00 | 0.00 | 0.00 | 0.00 |
| EMC1 | 0.00 | 0.00 | 0.00 | 0.00 | 0.00 | 0.00 | 0.00 | 0.00 | 0.00 | 0.00 |
| EOMES | 0.00 | 0.00 | 0.00 | 0.00 | 0.00 | 0.00 | 0.00 | 0.00 | 4.90 | 0.00 |
| EP300 | 0.00 | 0.00 | 0.00 | 0.00 | 3.39 | 0.00 | 0.00 | 0.00 | 0.00 | 0.00 |
| EPSTI1 | 0.00 | 0.00 | 6.09 | 0.00 | 0.00 | 0.00 | 0.00 | 0.00 | 0.00 | 0.00 |
| ERBB2IP | 0.00 | 0.00 | 0.00 | 0.00 | 0.00 | 4.07 | 0.00 | 0.00 | 0.00 | 0.00 |
| ERDR1 | 4.87 | 5.57 | 3.75 | 7.17 | 0.00 | 5.20 | 0.00 | 0.00 | 0.00 | 0.00 |
| ERGIC2 | 0.00 | 0.00 | 0.00 | 0.00 | 0.00 | 0.00 | 0.00 | 0.00 | 0.00 | 0.00 |
| ERO1L | 0.00 | 0.00 | 3.15 | 0.00 | 0.00 | 0.00 | 0.00 | 0.00 | 0.00 | 0.00 |
| ETS1 | 0.00 | 0.00 | 0.00 | 0.00 | 0.00 | 0.00 | 0.00 | 0.00 | 0.00 | 0.00 |
| ETS2 | 3.41 | 0.00 | 0.00 | 0.00 | 0.00 | 0.00 | 0.00 | 0.00 | 0.00 | 0.00 |
| ETV3 | 0.00 | 0.00 | 0.00 | 0.00 | 0.00 | 0.00 | 0.00 | 0.00 | 0.00 | 0.00 |
| EVI2A | 0.00 | 0.00 | 0.00 | 0.00 | 0.00 | 0.00 | 0.00 | 0.00 | 0.00 | 0.00 |
| EVI5L | 0.00 | 0.00 | 0.00 | 0.00 | 3.55 | 0.00 | 0.00 | 0.00 | 0.00 | 0.00 |
| FAM107B | 0.00 | 0.00 | 0.00 | 0.00 | 0.00 | 0.00 | 0.00 | 0.00 | 0.00 | 0.00 |
| FAM111A | 0.00 | 0.00 | 0.00 | 0.00 | 0.00 | 0.00 | 0.00 | 0.00 | 0.00 | 0.00 |
| FAM162A | 0.00 | 0.00 | 4.01 | 0.00 | 0.00 | 3.65 | 0.00 | 0.00 | 0.00 | 0.00 |
| FAM26F | 0.00 | 0.00 | 0.00 | 0.00 | 0.00 | 0.00 | 0.00 | 0.00 | 0.00 | 0.00 |
| FAM49A | 0.00 | 0.00 | 0.00 | 0.00 | 0.00 | 0.00 | 0.00 | 0.00 | 0.00 | 0.00 |
| FAM76B | 0.00 | 0.00 | 0.00 | 0.00 | 0.00 | 0.00 | 0.00 | 0.00 | 0.00 | 0.00 |
| FBXL3 | 0.00 | 0.00 | 0.00 | 0.00 | 0.00 | 0.00 | 4.28 | 0.00 | 0.00 | 0.00 |
| FCER1G | 4.93 | 0.00 | 0.00 | 0.00 | 5.34 | 4.56 | 4.58 | 0.00 | 0.00 | 0.00 |
| FCGR1 | 0.00 | 0.00 | 0.00 | 0.00 | 0.00 | 0.00 | 0.00 | 0.00 | 0.00 | 0.00 |
| FCGR3 | 6.11 | 0.00 | 0.00 | 0.00 | 0.00 | 0.00 | 0.00 | 0.00 | 0.00 | 0.00 |
| FCGR4 | 0.00 | 0.00 | 0.00 | 0.00 | 0.00 | 0.00 | 0.00 | 0.00 | 0.00 | 0.00 |
| FCMR | 0.00 | 0.00 | 0.00 | 0.00 | 0.00 | 0.00 | 0.00 | 0.00 | 0.00 | 4.08 |
| FGL2 | 0.00 | 0.00 | 0.00 | 0.00 | 0.00 | 0.00 | 0.00 | 0.00 | 0.00 | 0.00 |
| FIG4 | 0.00 | 0.00 | 0.00 | 0.00 | 0.00 | 0.00 | 0.00 | 0.00 | 0.00 | 0.00 |
| FILIP1L | 0.00 | 0.00 | 0.00 | 0.00 | 0.00 | 0.00 | 0.00 | 0.00 | 0.00 | 0.00 |
| FMNL2 | 0.00 | 0.00 | 0.00 | 0.00 | 0.00 | 0.00 | 0.00 | 0.00 | 0.00 | 0.00 |
| FMR1 | 0.00 | 0.00 | 0.00 | 0.00 | 3.49 | 0.00 | 0.00 | 0.00 | 0.00 | 0.00 |
| FOSB | 0.00 | 0.00 | 0.00 | 0.00 | 0.00 | 0.00 | 0.00 | 0.00 | 0.00 | 0.00 |
| FOXP3 | 0.00 | 0.00 | 0.00 | 0.00 | 0.00 | 0.00 | 0.00 | 0.00 | 0.00 | 4.34 |
| FPR2 | 0.00 | 0.00 | 0.00 | 0.00 | 0.00 | 0.00 | 0.00 | 0.00 | 0.00 | 0.00 |
| FRAT2 | 0.00 | 0.00 | 0.00 | 0.00 | 0.00 | 0.00 | 0.00 | 0.00 | 0.00 | 0.00 |
| FRMD4A | 0.00 | 0.00 | 0.00 | 0.00 | 0.00 | 0.00 | 0.00 | 0.00 | 0.00 | 0.00 |
| FTH1 | 0.00 | 0.00 | 0.00 | 0.00 | 0.00 | 0.00 | 0.00 | 0.00 | 0.00 | 0.00 |
| FTL1 | 0.00 | 0.00 | 0.00 | 0.00 | 0.00 | 0.00 | 0.00 | 0.00 | 0.00 | 0.00 |
| FURIN | 0.00 | 0.00 | 0.00 | 0.00 | 0.00 | 0.00 | 0.00 | 0.00 | 0.00 | 0.00 |
| FYB | 0.00 | 0.00 | 0.00 | 0.00 | 0.00 | 0.00 | 0.00 | 0.00 | 0.00 | 0.00 |
| GOS2 | 0.00 | 0.00 | 0.00 | 0.00 | 0.00 | 0.00 | 0.00 | 0.00 | 0.00 | 0.00 |
| GADD45A | 0.00 | 0.00 | 0.00 | 0.00 | 0.00 | 0.00 | 0.00 | 0.00 | 0.00 | 0.00 |
| GADD45B | 0.00 | 0.00 | 0.00 | 0.00 | 0.00 | 0.00 | 0.00 | 0.00 | 0.00 | 0.00 |
| GADD45G | 4.43 | 0.00 | 0.00 | 19.00 | 0.00 | 0.00 | 0.00 | 0.00 | 0.00 | 0.00 |
| GAPDH | 0.00 | 0.00 | 0.00 | 0.00 | 0.00 | 4.26 | 0.00 | 0.00 | 0.00 | 0.00 |
| GAS2 | 0.00 | 0.00 | 4.87 | 0.00 | 0.00 | 0.00 | 0.00 | 0.00 | 0.00 | 0.00 |
| GBE1 | 0.00 | 0.00 | 3.13 | 0.00 | 0.00 | 0.00 | 0.00 | 0.00 | 0.00 | 0.00 |
| GBP2 | 0.00 | 0.00 | 5.70 | 0.00 | 0.00 | 0.00 | 0.00 | 0.00 | 0.00 | 0.00 |
| GBP3 | 0.00 | 0.00 | 5.29 | 0.00 | 0.00 | 0.00 | 0.00 | 0.00 | 0.00 | 0.00 |
| GBP4 | 0.00 | 0.00 | 6.81 | 0.00 | 0.00 | 0.00 | 0.00 | 0.00 | 0.00 | 0.00 |
| GBP5 | 0.00 | 0.00 | 0.00 | 0.00 | 0.00 | 0.00 | 0.00 | 0.00 | 0.00 | 0.00 |
| GBP7 | 0.00 | 0.00 | 4.45 | 0.00 | 0.00 | 0.00 | 0.00 | 0.00 | 0.00 | 0.00 |
| GBP8 | 0.00 | 0.00 | 0.00 | 0.00 | 0.00 | 0.00 | 0.00 | 0.00 | 0.00 | 0.00 |
| GBP9 | 0.00 | 0.00 | 3.53 | 0.00 | 0.00 | 0.00 | 0.00 | 0.00 | 0.00 | 0.00 |
| GCNT2 | 0.00 | 0.00 | 0.00 | 0.00 | 0.00 | 0.00 | 0.00 | 0.00 | 0.00 | 0.00 |
| GDPGP1 | 0.00 | 0.00 | 0.00 | 0.00 | 3.58 | 0.00 | 0.00 | 0.00 | 0.00 | 0.00 |
| GEM | 0.00 | 0.00 | 0.00 | 0.00 | 0.00 | 0.00 | 0.00 | 0.00 | 0.00 | 0.00 |
| GIMAP3 | 0.00 | 0.00 | 3.31 | 0.00 | 0.00 | 0.00 | 0.00 | 0.00 | 0.00 | 0.00 |
| GIMAP4 | 0.00 | 0.00 | 5.58 | 0.00 | 0.00 | 0.00 | 0.00 | 0.00 | 0.00 | 0.00 |
| GIMAP5 | 0.00 | 0.00 | 0.00 | 0.00 | 0.00 | 0.00 | 0.00 | 0.00 | 0.00 | 0.00 |
| GIMAP8 | 0.00 | 0.00 | 0.00 | 0.00 | 0.00 | 0.00 | 0.00 | 0.00 | 0.00 | 0.00 |
| GLA | 0.00 | 0.00 | 0.00 | 0.00 | 0.00 | 0.00 | 0.00 | 0.00 | 0.00 | 0.00 |
| GM12185 | 0.00 | 0.00 | 0.00 | 0.00 | 3.35 | 0.00 | 0.00 | 0.00 | 0.00 | 0.00 |
| GM12216 | 0.00 | 0.00 | 0.00 | 0.00 | 0.00 | 0.00 | 0.00 | 0.00 | 0.00 | 0.00 |
| GM12840 | 0.00 | 0.00 | 0.00 | 0.00 | 4.23 | 0.00 | 0.00 | 0.00 | 0.00 | 0.00 |
| GM15987 | 0.00 | 0.00 | 0.00 | 0.00 | 0.00 | 0.00 | 0.00 | 0.00 | 0.00 | 0.00 |
| GM2000 | 4.25 | 0.00 | 0.00 | 4.93 | 0.00 | 0.00 | 0.00 | 0.00 | 0.00 | 0.00 |
| GM26545 | 0.00 | 0.00 | 0.00 | 0.00 | 0.00 | 5.09 | 0.00 | 0.00 | 0.00 | 0.00 |
| GM26699 | 4.94 | 0.00 | 0.00 | 0.00 | 0.00 | 0.00 | 0.00 | 0.00 | 0.00 | 0.00 |
| GM26917 | 0.00 | 0.00 | 0.00 | 0.00 | 0.00 | 4.61 | 0.00 | 0.00 | 0.00 | 0.00 |
| GM37065 | 0.00 | 0.00 | 0.00 | 0.00 | 3.33 | 0.00 | 0.00 | 0.00 | 0.00 | 0.00 |
| GM43603 | 0.00 | 0.00 | 3.67 | 0.00 | 0.00 | 0.00 | 0.00 | 0.00 | 0.00 | 0.00 |
| GM4951 | 0.00 | 0.00 | 4.34 | 0.00 | 0.00 | 0.00 | 0.00 | 0.00 | 0.00 | 0.00 |
| GM8369 | 5.26 | 0.00 | 0.00 | 0.00 | 0.00 | 0.00 | 0.00 | 0.00 | 0.00 | 0.00 |
| GM8797 | 0.00 | 0.00 | 0.00 | 4.55 | 0.00 | 0.00 | 0.00 | 0.00 | 0.00 | 0.00 |
| GM8953 | 0.00 | 0.00 | 0.00 | 0.00 | 0.00 | 0.00 | 0.00 | 0.00 | 0.00 | 0.00 |
| GNB2L1 | 0.00 | 0.00 | 0.00 | 0.00 | 0.00 | 0.00 | 0.00 | 0.00 | 0.00 | 0.00 |
| GOLGA1 | 0.00 | 0.00 | 0.00 | 0.00 | 3.88 | 0.00 | 0.00 | 0.00 | 0.00 | 0.00 |
| GOLGA3 | 0.00 | 0.00 | 0.00 | 0.00 | 0.00 | 0.00 | 0.00 | 0.00 | 0.00 | 0.00 |
| GPATCH2 | 0.00 | 0.00 | 0.00 | 0.00 | 0.00 | 0.00 | 0.00 | 0.00 | 0.00 | 0.00 |
| GPI1 | 0.00 | 0.00 | 0.00 | 0.00 | 0.00 | 0.00 | 0.00 | 0.00 | 0.00 | 0.00 |
| GPNMB | 0.00 | 0.00 | 0.00 | 0.00 | 0.00 | 0.00 | 0.00 | 0.00 | 0.00 | 0.00 |
| GPR35 | 0.00 | 0.00 | 0.00 | 0.00 | 0.00 | 0.00 | 0.00 | 0.00 | 0.00 | 0.00 |
| GRINA | 0.00 | 0.00 | 0.00 | 0.00 | 0.00 | 0.00 | 0.00 | 0.00 | 0.00 | 0.00 |
| GSR | 0.00 | 0.00 | 0.00 | 0.00 | 0.00 | 0.00 | 0.00 | 0.00 | 0.00 | 0.00 |
| GZMA | 0.00 | 0.00 | 0.00 | 5.25 | 0.00 | 0.00 | 0.00 | 0.00 | 0.00 | 0.00 |
| GZMB | 0.00 | 0.00 | 0.00 | 0.00 | 0.00 | 9.50 | 0.00 | 0.00 | 0.00 | 0.00 |
| GZMC | 0.00 | 0.00 | 0.00 | 0.00 | 0.00 | 0.00 | 0.00 | 0.00 | 0.00 | 0.00 |
| GZMF | 0.00 | 0.00 | 0.00 | 0.00 | 0.00 | 0.00 | 0.00 | 0.00 | 0.00 | 6.00 |
| H2-AA | 0.00 | 4.38 | 0.00 | 0.00 | 0.00 | 0.00 | 0.00 | 0.00 | 0.00 | 0.00 |
| H2-AB1 | 7.39 | 0.00 | 0.00 | 0.00 | 0.00 | 6.13 | 6.11 | 0.00 | 0.00 | 0.00 |
| H2AFY | 0.00 | 0.00 | 0.00 | 0.00 | 0.00 | 0.00 | 0.00 | 0.00 | 0.00 | 0.00 |
| H2AFZ | 0.00 | 0.00 | 0.00 | 0.00 | 0.00 | 0.00 | 0.00 | 0.00 | 0.00 | 0.00 |
| H2-D1 | 0.00 | 0.00 | 0.00 | 0.00 | 0.00 | 0.00 | 0.00 | 0.00 | 0.00 | 0.00 |
| H2-DMA | 0.00 | 0.00 | 0.00 | 0.00 | 0.00 | 0.00 | 0.00 | 0.00 | 0.00 | 0.00 |
| H2-DMB1 | 0.00 | 0.00 | 0.00 | 0.00 | 0.00 | 0.00 | 0.00 | 0.00 | 0.00 | 0.00 |
| H2-DMB2 | 0.00 | 0.00 | 3.35 | 0.00 | 0.00 | 0.00 | 0.00 | 0.00 | 0.00 | 0.00 |
| H2-K1 | 0.00 | 0.00 | 4.21 | 0.00 | 0.00 | 0.00 | 0.00 | 0.00 | 0.00 | 0.00 |
| H2-M3 | 0.00 | 0.00 | 0.00 | 0.00 | 0.00 | 0.00 | 0.00 | 0.00 | 0.00 | 0.00 |
| H2-OB | 0.00 | 0.00 | 3.13 | 0.00 | 0.00 | 0.00 | 0.00 | 0.00 | 0.00 | 0.00 |
| H2-Q10 | 0.00 | 0.00 | 0.00 | 0.00 | 0.00 | 0.00 | 0.00 | 0.00 | 0.00 | 0.00 |
| H2-Q4 | 0.00 | 0.00 | 0.00 | 0.00 | 0.00 | 0.00 | 0.00 | 0.00 | 0.00 | 0.00 |
| H2-Q6 | 0.00 | 0.00 | 0.00 | 0.00 | 0.00 | 0.00 | 0.00 | 0.00 | 0.00 | 0.00 |
| H2-Q7 | 0.00 | 0.00 | 5.08 | 0.00 | 0.00 | 0.00 | 0.00 | 0.00 | 0.00 | 0.00 |
| H2-T22 | 0.00 | 0.00 | 3.43 | 0.00 | 0.00 | 0.00 | 0.00 | 0.00 | 0.00 | 0.00 |
| H2-T23 | 0.00 | 0.00 | 0.00 | 0.00 | 0.00 | 0.00 | 0.00 | 0.00 | 0.00 | 0.00 |
| HADHA | 0.00 | 0.00 | 0.00 | 0.00 | 0.00 | 0.00 | 0.00 | 0.00 | 0.00 | 0.00 |
| HBA-A2 | 0.00 | 0.00 | 3.94 | 0.00 | 0.00 | 0.00 | 0.00 | 0.00 | 0.00 | 0.00 |
| HBB-BT | 0.00 | 0.00 | 3.63 | 0.00 | 0.00 | 0.00 | 0.00 | 0.00 | 0.00 | 0.00 |
| HCAR2 | 6.19 | 0.00 | 0.00 | 0.00 | 0.00 | 0.00 | 0.00 | 0.00 | 0.00 | 0.00 |
| HCK | 0.00 | 0.00 | 0.00 | 0.00 | 0.00 | 0.00 | 0.00 | 0.00 | 0.00 | 0.00 |
| HDC | 0.00 | 0.00 | 0.00 | 0.00 | 0.00 | 0.00 | 0.00 | 0.00 | 0.00 | 0.00 |
| HDLBP | 0.00 | 0.00 | 0.00 | 0.00 | 5.04 | 0.00 | 0.00 | 0.00 | 0.00 | 0.00 |
| HEATR5A | 0.00 | 0.00 | 0.00 | 0.00 | 0.00 | 0.00 | 0.00 | 0.00 | 0.00 | 0.00 |
| HEATR5B | 0.00 | 0.00 | 3.57 | 0.00 | 0.00 | 0.00 | 0.00 | 0.00 | 0.00 | 0.00 |
| HELQ | 0.00 | 0.00 | 0.00 | 0.00 | 6.01 | 0.00 | 0.00 | 0.00 | 0.00 | 0.00 |
| HERC6 | 0.00 | 0.00 | 0.00 | 0.00 | 0.00 | 0.00 | 0.00 | 0.00 | 0.00 | 0.00 |
| HES1 | 0.00 | 0.00 | 0.00 | 0.00 | 0.00 | 0.00 | 0.00 | 0.00 | 0.00 | 0.00 |
| HID1 | 0.00 | 0.00 | 0.00 | 0.00 | 0.00 | 0.00 | 0.00 | 0.00 | 0.00 | 0.00 |
| HILPDA | 0.00 | 0.00 | 0.00 | 0.00 | 0.00 | 0.00 | 0.00 | 0.00 | 0.00 | 0.00 |
| HIPK2 | 0.00 | 0.00 | 0.00 | 0.00 | 0.00 | 0.00 | 0.00 | 0.00 | 0.00 | 0.00 |
| HIST1H1C | 0.00 | 0.00 | 0.00 | 0.00 | 0.00 | 0.00 | 0.00 | 0.00 | 0.00 | 0.00 |
| HIST1H1E | 3.45 | 0.00 | 0.00 | 0.00 | 0.00 | 0.00 | 0.00 | 0.00 | 4.24 | 0.00 |
| HIST1H2AE | 0.00 | 0.00 | 0.00 | 0.00 | 0.00 | 0.00 | 0.00 | 0.00 | 0.00 | 0.00 |
| HIST1H2AG | 0.00 | 0.00 | 0.00 | 0.00 | 3.44 | 0.00 | 0.00 | 0.00 | 0.00 | 0.00 |
| HIST1H2BC | 0.00 | 0.00 | 0.00 | 0.00 | 0.00 | 0.00 | 0.00 | 0.00 | 0.00 | 0.00 |
| HIST1H2BJ | 0.00 | 0.00 | 0.00 | 0.00 | 5.59 | 0.00 | 0.00 | 0.00 | 0.00 | 0.00 |
| HIST1H4I | 0.00 | 0.00 | 0.00 | 0.00 | 0.00 | 0.00 | 0.00 | 0.00 | 0.00 | 0.00 |
| HNRNPU | 0.00 | 0.00 | 0.00 | 0.00 | 3.86 | 0.00 | 0.00 | 0.00 | 0.00 | 0.00 |
| HOXA7 | 0.00 | 0.00 | 0.00 | 0.00 | 5.01 | 0.00 | 0.00 | 0.00 | 0.00 | 0.00 |
| HOXB4 | 0.00 | 0.00 | 0.00 | 0.00 | 0.00 | 0.00 | 0.00 | 0.00 | 0.00 | 0.00 |
| HP | 0.00 | 0.00 | 0.00 | 0.00 | 0.00 | 0.00 | 0.00 | 0.00 | 0.00 | 0.00 |
| HP1BP3 | 0.00 | 0.00 | 0.00 | 0.00 | 0.00 | 0.00 | 0.00 | 0.00 | 0.00 | 0.00 |
| HPGDS | 0.00 | 0.00 | 5.78 | 0.00 | 0.00 | 0.00 | 0.00 | 0.00 | 0.00 | 0.00 |
| HSP90AA1 | 0.00 | 0.00 | 0.00 | 3.76 | 0.00 | 0.00 | 0.00 | 0.00 | 0.00 | 0.00 |
| HSP90AB1 | 0.00 | 0.00 | 0.00 | 0.00 | 0.00 | 0.00 | 0.00 | 0.00 | 0.00 | 0.00 |
| HSPA14 | 4.02 | 0.00 | 0.00 | 0.00 | 0.00 | 0.00 | 0.00 | 0.00 | 0.00 | 0.00 |
| HSPA1A | 0.00 | 0.00 | 0.00 | 21.98 | 0.00 | 0.00 | 0.00 | 0.00 | 11.68 | 0.00 |
| HSPA1B | 0.00 | 0.00 | 0.00 | 11.79 | 0.00 | 0.00 | 0.00 | 0.00 | 0.00 | 0.00 |
| HSPA8 | 0.00 | 0.00 | 0.00 | 0.00 | 0.00 | 0.00 | 0.00 | 0.00 | 0.00 | 0.00 |
| HSPD1 | 0.00 | 0.00 | 0.00 | 0.00 | 0.00 | 0.00 | 0.00 | 0.00 | 0.00 | 0.00 |
| HYAL2 | 4.08 | 0.00 | 0.00 | 0.00 | 0.00 | 0.00 | 0.00 | 0.00 | 0.00 | 0.00 |
| ICAM1 | 0.00 | 0.00 | 0.00 | 0.00 | 0.00 | 0.00 | 0.00 | 0.00 | 0.00 | 0.00 |
| ICOS | 0.00 | 0.00 | 0.00 | 0.00 | 0.00 | 0.00 | 0.00 | 0.00 | 0.00 | 0.00 |
| ID1 | 0.00 | 0.00 | 0.00 | 0.00 | 0.00 | 0.00 | 0.00 | 0.00 | 0.00 | 0.00 |
| ID2 | 0.00 | 0.00 | 5.31 | 4.56 | 0.00 | 5.20 | 0.00 | 0.00 | 0.00 | 0.00 |
| ID3 | 0.00 | 0.00 | 0.00 | 6.43 | 0.00 | 0.00 | 0.00 | 0.00 | 0.00 | 0.00 |
| IER2 | 4.61 | 0.00 | 0.00 | 4.85 | 0.00 | 0.00 | 0.00 | 0.00 | 0.00 | 0.00 |
| IER3 | 0.00 | 0.00 | 0.00 | 0.00 | 0.00 | 0.00 | 0.00 | 0.00 | 0.00 | 0.00 |
| IER5 | 0.00 | 0.00 | 0.00 | 0.00 | 0.00 | 0.00 | 0.00 | 0.00 | 0.00 | 0.00 |
| IFI203 | 4.81 | 0.00 | 4.67 | 0.00 | 0.00 | 0.00 | 0.00 | 0.00 | 0.00 | 0.00 |
| IFI204 | 0.00 | 0.00 | 0.00 | 0.00 | 0.00 | 0.00 | 0.00 | 0.00 | 0.00 | 0.00 |
| IFI205 | 0.00 | 0.00 | 0.00 | 0.00 | 0.00 | 0.00 | 0.00 | 0.00 | 0.00 | 0.00 |
| IFI27L2A | 7.49 | 0.00 | 15.22 | 0.00 | 0.00 | 8.58 | 0.00 | 0.00 | 4.93 | 4.84 |
| IFI35 | 0.00 | 0.00 | 0.00 | 0.00 | 0.00 | 0.00 | 0.00 | 0.00 | 0.00 | 0.00 |
| IFI44 | 0.00 | 0.00 | 0.00 | 0.00 | 0.00 | 0.00 | 0.00 | 0.00 | 0.00 | 0.00 |
| IFI47 | 0.00 | 0.00 | 7.45 | 0.00 | 0.00 | 4.72 | 0.00 | 0.00 | 0.00 | 0.00 |
| IFIT1 | 3.51 | 4.04 | 0.00 | 0.00 | 0.00 | 7.88 | 0.00 | 0.00 | 0.00 | 0.00 |
| IFIT2 | 0.00 | 0.00 | 0.00 | 0.00 | 0.00 | 0.00 | 0.00 | 0.00 | 0.00 | 0.00 |
| IFIT3 | 0.00 | 6.66 | 0.00 | 0.00 | 0.00 | 6.36 | 0.00 | 0.00 | 0.00 | 0.00 |
| IFIT3B | 0.00 | 6.19 | 0.00 | 0.00 | 0.00 | 0.00 | 0.00 | 0.00 | 0.00 | 0.00 |
| IFITM1 | 0.00 | 4.52 | 0.00 | 0.00 | 0.00 | 0.00 | 0.00 | 0.00 | 0.00 | 0.00 |
| IFITM2 | 0.00 | 0.00 | 0.00 | 0.00 | 0.00 | 0.00 | 0.00 | 0.00 | 0.00 | 4.38 |
| IFITM6 | 0.00 | 0.00 | 0.00 | 0.00 | 0.00 | 0.00 | 0.00 | 0.00 | 0.00 | 0.00 |
| IFNG | 0.00 | 0.00 | 3.62 | 0.00 | 0.00 | 0.00 | 0.00 | 0.00 | 0.00 | 0.00 |
| IFNGR1 | 0.00 | 0.00 | 0.00 | 0.00 | 0.00 | 0.00 | 0.00 | 0.00 | 0.00 | 4.16 |
| IFRD1 | 0.00 | 0.00 | 0.00 | 0.00 | 0.00 | 0.00 | 0.00 | 0.00 | 0.00 | 0.00 |
| IFT22 | 0.00 | 0.00 | 0.00 | 0.00 | 0.00 | 0.00 | 0.00 | 0.00 | 0.00 | 0.00 |
| IGF2R | 0.00 | 0.00 | 0.00 | 0.00 | 3.66 | 0.00 | 0.00 | 0.00 | 0.00 | 0.00 |
| IGFBP7 | 0.00 | 0.00 | 0.00 | 0.00 | 0.00 | 0.00 | 0.00 | 0.00 | 0.00 | 0.00 |
| IGSF23 | 0.00 | 0.00 | 3.82 | 0.00 | 0.00 | 0.00 | 0.00 | 0.00 | 0.00 | 0.00 |
| IGTP | 3.81 | 4.35 | 9.94 | 0.00 | 0.00 | 4.89 | 0.00 | 0.00 | 0.00 | 0.00 |
| IIGP1 | 4.58 | 4.55 | 6.01 | 0.00 | 0.00 | 8.77 | 0.00 | 0.00 | 0.00 | 0.00 |
| IKBKE | 0.00 | 0.00 | 0.00 | 0.00 | 0.00 | 0.00 | 0.00 | 0.00 | 0.00 | 0.00 |
| IL10RA | 0.00 | 0.00 | 0.00 | 0.00 | 0.00 | 0.00 | 0.00 | 0.00 | 0.00 | 0.00 |
| IL18BP | 0.00 | 0.00 | 0.00 | 0.00 | 0.00 | 0.00 | 0.00 | 0.00 | 0.00 | 0.00 |
| IL1B | 0.00 | 0.00 | 0.00 | 0.00 | 0.00 | 0.00 | 0.00 | 0.00 | 0.00 | 0.00 |
| IL1R2 | 0.00 | 0.00 | 0.00 | 0.00 | 0.00 | 0.00 | 0.00 | 0.00 | 0.00 | 0.00 |
| IL1RN | 0.00 | 0.00 | 0.00 | 0.00 | 0.00 | 0.00 | 0.00 | 0.00 | 0.00 | 0.00 |
| IL3RA | 0.00 | 0.00 | 0.00 | 0.00 | 0.00 | 0.00 | 0.00 | 0.00 | 0.00 | 0.00 |
| IL4I1 | 0.00 | 0.00 | 0.00 | 0.00 | 0.00 | 0.00 | 0.00 | 0.00 | 0.00 | 4.66 |
| INO80 | 0.00 | 0.00 | 0.00 | 0.00 | 8.33 | 0.00 | 0.00 | 0.00 | 0.00 | 0.00 |
| INSIG1 | 0.00 | 0.00 | 0.00 | 0.00 | 0.00 | 0.00 | 0.00 | 0.00 | 0.00 | 0.00 |
| IPCEF1 | 0.00 | 0.00 | 0.00 | 0.00 | 3.90 | 0.00 | 0.00 | 0.00 | 0.00 | 0.00 |
| IQGAP1 | 0.00 | 0.00 | 0.00 | 0.00 | 0.00 | 0.00 | 0.00 | 0.00 | 0.00 | 0.00 |
| IQGAP2 | 0.00 | 0.00 | 0.00 | 0.00 | 4.31 | 0.00 | 0.00 | 0.00 | 0.00 | 0.00 |
| IRAK2 | 0.00 | 0.00 | 0.00 | 0.00 | 0.00 | 0.00 | 0.00 | 0.00 | 0.00 | 0.00 |
| IRF1 | 0.00 | 0.00 | 6.22 | 0.00 | 0.00 | 0.00 | 0.00 | 0.00 | 0.00 | 0.00 |
| IRF7 | 0.00 | 0.00 | 5.73 | 0.00 | 0.00 | 0.00 | 0.00 | 0.00 | 0.00 | 0.00 |
| IRF8 | 0.00 | 0.00 | 0.00 | 0.00 | 0.00 | 0.00 | 0.00 | 0.00 | 0.00 | 0.00 |
| IRF9 | 0.00 | 0.00 | 9.45 | 0.00 | 0.00 | 0.00 | 0.00 | 0.00 | 0.00 | 0.00 |
| IRG1 | 3.56 | 0.00 | 0.00 | 0.00 | 0.00 | 0.00 | 0.00 | 0.00 | 0.00 | 0.00 |
| IRGM1 | 0.00 | 0.00 | 0.00 | 0.00 | 0.00 | 0.00 | 0.00 | 0.00 | 0.00 | 0.00 |
| IRGM2 | 0.00 | 0.00 | 0.00 | 0.00 | 0.00 | 0.00 | 0.00 | 0.00 | 0.00 | 0.00 |
| ISG20 | 0.00 | 0.00 | 0.00 | 0.00 | 0.00 | 0.00 | 0.00 | 0.00 | 0.00 | 0.00 |
| ITGA4 | 0.00 | 0.00 | 0.00 | 0.00 | 3.33 | 0.00 | 0.00 | 0.00 | 0.00 | 0.00 |
| ITGB1 | 0.00 | 0.00 | 5.07 | 0.00 | 0.00 | 0.00 | 0.00 | 0.00 | 0.00 | 0.00 |
| ITIH5 | 0.00 | 0.00 | 3.22 | 0.00 | 0.00 | 0.00 | 0.00 | 0.00 | 0.00 | 0.00 |
| ITM2A | 0.00 | 0.00 | 0.00 | 0.00 | 0.00 | 0.00 | 0.00 | 0.00 | 0.00 | 0.00 |
| ITM2B | 0.00 | 0.00 | 0.00 | 0.00 | 0.00 | 0.00 | 0.00 | 0.00 | 0.00 | 0.00 |
| ITSN1 | 0.00 | 0.00 | 0.00 | 0.00 | 0.00 | 0.00 | 0.00 | 0.00 | 0.00 | 0.00 |
| JAKMIP1 | 0.00 | 0.00 | 0.00 | 0.00 | 0.00 | 0.00 | 0.00 | 0.00 | 0.00 | 0.00 |
| JUN | 5.95 | 0.00 | 0.00 | 7.85 | 0.00 | 0.00 | 0.00 | 0.00 | 0.00 | 0.00 |
| JUNB | 3.99 | 0.00 | 0.00 | 8.99 | 0.00 | 0.00 | 0.00 | 0.00 | 0.00 | 0.00 |
| KDM6B | 0.00 | 0.00 | 0.00 | 0.00 | 0.00 | 0.00 | 0.00 | 0.00 | 0.00 | 0.00 |
| KIF20B | 0.00 | 0.00 | 3.51 | 0.00 | 0.00 | 0.00 | 0.00 | 0.00 | 0.00 | 0.00 |
| KIF3B | 0.00 | 0.00 | 0.00 | 0.00 | 0.00 | 0.00 | 0.00 | 0.00 | 0.00 | 0.00 |
| KIFC1 | 0.00 | 0.00 | 4.76 | 0.00 | 0.00 | 0.00 | 0.00 | 0.00 | 0.00 | 0.00 |
| KLC4 | 0.00 | 0.00 | 0.00 | 0.00 | 0.00 | 0.00 | 0.00 | 0.00 | 0.00 | 0.00 |
| KLF2 | 3.30 | 0.00 | 0.00 | 0.00 | 0.00 | 0.00 | 0.00 | 0.00 | 0.00 | 0.00 |
| KLF6 | 0.00 | 0.00 | 0.00 | 0.00 | 0.00 | 0.00 | 0.00 | 0.00 | 0.00 | 0.00 |
| KLF9 | 0.00 | 0.00 | 0.00 | 0.00 | 0.00 | 0.00 | 0.00 | 0.00 | 0.00 | 0.00 |
| KLK8 | 0.00 | 0.00 | 0.00 | 0.00 | 0.00 | 0.00 | 0.00 | 0.00 | 0.00 | 0.00 |
| KLRA4 | 0.00 | 0.00 | 0.00 | 0.00 | 0.00 | 0.00 | 0.00 | 0.00 | 0.00 | 0.00 |
| KLRA8 | 0.00 | 0.00 | 0.00 | 0.00 | 0.00 | 0.00 | 0.00 | 0.00 | 0.00 | 0.00 |
| KLRA9 | 0.00 | 0.00 | 0.00 | 0.00 | 3.79 | 0.00 | 0.00 | 0.00 | 0.00 | 0.00 |
| KLRC1 | 0.00 | 0.00 | 0.00 | 0.00 | 0.00 | 7.14 | 0.00 | 0.00 | 0.00 | 0.00 |
| KLRD1 | 0.00 | 0.00 | 0.00 | 0.00 | 0.00 | 4.44 | 0.00 | 0.00 | 0.00 | 0.00 |
| KLRK1 | 0.00 | 0.00 | 0.00 | 0.00 | 0.00 | 0.00 | 0.00 | 0.00 | 0.00 | 0.00 |
| KMO | 0.00 | 0.00 | 0.00 | 0.00 | 0.00 | 0.00 | 0.00 | 0.00 | 0.00 | 0.00 |
| KMT2E | 0.00 | 0.00 | 0.00 | 0.00 | 3.87 | 0.00 | 0.00 | 0.00 | 0.00 | 0.00 |
| LACC1 | 0.00 | 0.00 | 0.00 | 0.00 | 0.00 | 0.00 | 0.00 | 0.00 | 0.00 | 0.00 |
| LAPTM5 | 0.00 | 0.00 | 0.00 | 0.00 | 0.00 | 0.00 | 0.00 | 0.00 | 0.00 | 0.00 |
| LCK | 0.00 | 0.00 | 0.00 | 0.00 | 0.00 | 0.00 | 0.00 | 0.00 | 0.00 | 0.00 |
| LCP1 | 3.30 | 0.00 | 0.00 | 0.00 | 0.00 | 0.00 | 0.00 | 0.00 | 0.00 | 0.00 |
| LDHA | 0.00 | 0.00 | 0.00 | 0.00 | 0.00 | 0.00 | 0.00 | 0.00 | 0.00 | 0.00 |
| LEPR | 0.00 | 0.00 | 0.00 | 0.00 | 0.00 | 0.00 | 0.00 | 0.00 | 0.00 | 0.00 |
| LGALS3 | 0.00 | 0.00 | 0.00 | 0.00 | 0.00 | 0.00 | 0.00 | 0.00 | 0.00 | 0.00 |
| LGALS3BP | 0.00 | 0.00 | 0.00 | 0.00 | 0.00 | 0.00 | 0.00 | 0.00 | 0.00 | 0.00 |
| LGALS9 | 0.00 | 0.00 | 0.00 | 0.00 | 0.00 | 0.00 | 0.00 | 0.00 | 0.00 | 0.00 |
| LILR4B | 0.00 | 0.00 | 0.00 | 0.00 | 0.00 | 0.00 | 0.00 | 0.00 | 0.00 | 0.00 |
| LILRB4A | 0.00 | 0.00 | 0.00 | 0.00 | 0.00 | 0.00 | 0.00 | 0.00 | 0.00 | 0.00 |
| LITAF | 0.00 | 0.00 | 0.00 | 0.00 | 0.00 | 0.00 | 0.00 | 0.00 | 0.00 | 0.00 |
| LMBRD2 | 0.00 | 0.00 | 0.00 | 0.00 | 5.19 | 0.00 | 0.00 | 0.00 | 0.00 | 0.00 |
| LMNB1 | 0.00 | 0.00 | 0.00 | 0.00 | 0.00 | 0.00 | 0.00 | 0.00 | 0.00 | 0.00 |
| LMO4 | 0.00 | 0.00 | 3.34 | 0.00 | 0.00 | 0.00 | 0.00 | 0.00 | 0.00 | 0.00 |
| LRBA | 0.00 | 0.00 | 3.46 | 0.00 | 0.00 | 0.00 | 0.00 | 0.00 | 0.00 | 0.00 |
| LRG1 | 0.00 | 0.00 | 0.00 | 0.00 | 0.00 | 0.00 | 0.00 | 0.00 | 0.00 | 0.00 |
| LRRC25 | 0.00 | 0.00 | 0.00 | 0.00 | 0.00 | 0.00 | 0.00 | 0.00 | 0.00 | 0.00 |
| LRRK1 | 0.00 | 0.00 | 0.00 | 0.00 | 0.00 | 0.00 | 0.00 | 0.00 | 0.00 | 0.00 |
| LSM12 | 0.00 | 0.00 | 0.00 | 0.00 | 0.00 | 0.00 | 0.00 | 0.00 | 0.00 | 0.00 |
| LSP1 | 0.00 | 0.00 | 0.00 | 0.00 | 0.00 | 0.00 | 0.00 | 0.00 | 0.00 | 0.00 |
| LTA | 0.00 | 0.00 | 0.00 | 0.00 | 0.00 | 0.00 | 0.00 | 0.00 | 0.00 | 0.00 |
| LY6A | 0.00 | 4.27 | 4.18 | 4.97 | 4.94 | 0.00 | 0.00 | 0.00 | 0.00 | 6.26 |
| LY6C2 | 7.62 | 0.00 | 0.00 | 5.40 | 0.00 | 0.00 | 0.00 | 0.00 | 0.00 | 0.00 |
| LY6D | 0.00 | 0.00 | 0.00 | 0.00 | 0.00 | 0.00 | 0.00 | 0.00 | 0.00 | 8.47 |
| LY6E | 4.19 | 0.00 | 0.00 | 0.00 | 0.00 | 0.00 | 0.00 | 0.00 | 0.00 | 4.99 |
| LY6I | 0.00 | 0.00 | 0.00 | 0.00 | 0.00 | 0.00 | 0.00 | 0.00 | 0.00 | 0.00 |
| LY86 | 0.00 | 0.00 | 0.00 | 0.00 | 0.00 | 0.00 | 0.00 | 0.00 | 0.00 | 0.00 |
| LYSMD3 | 0.00 | 0.00 | 0.00 | 0.00 | 4.32 | 0.00 | 0.00 | 0.00 | 0.00 | 0.00 |
| LYZ1 | 0.00 | 0.00 | 0.00 | 0.00 | 0.00 | 0.00 | 0.00 | 0.00 | 0.00 | 0.00 |
| MACROD1 | 0.00 | 0.00 | 0.00 | 0.00 | 3.95 | 0.00 | 0.00 | 0.00 | 0.00 | 0.00 |
| MAF | 0.00 | 0.00 | 0.00 | 0.00 | 0.00 | 0.00 | 0.00 | 0.00 | 0.00 | 0.00 |
| MAFF | 0.00 | 0.00 | 0.00 | 0.00 | 0.00 | 0.00 | 0.00 | 0.00 | 0.00 | 0.00 |
| MAP3K8 | 0.00 | 0.00 | 0.00 | 5.11 | 0.00 | 0.00 | 0.00 | 0.00 | 0.00 | 0.00 |
| MARCH5 | 0.00 | 0.00 | 0.00 | 0.00 | 0.00 | 0.00 | 0.00 | 0.00 | 0.00 | 0.00 |
| MARCKS | 0.00 | 0.00 | 0.00 | 0.00 | 0.00 | 0.00 | 0.00 | 0.00 | 0.00 | 0.00 |
| MARCKSL1 | 0.00 | 0.00 | 0.00 | 0.00 | 0.00 | 0.00 | 0.00 | 0.00 | 0.00 | 0.00 |
| MASTL | 0.00 | 0.00 | 0.00 | 0.00 | 0.00 | 0.00 | 0.00 | 0.00 | 0.00 | 0.00 |
| MBNL2 | 0.00 | 0.00 | 0.00 | 0.00 | 0.00 | 0.00 | 0.00 | 0.00 | 0.00 | 0.00 |
| MEF2C | 0.00 | 0.00 | 4.79 | 0.00 | 0.00 | 0.00 | 0.00 | 0.00 | 5.09 | 0.00 |
| MEFV | 0.00 | 0.00 | 0.00 | 0.00 | 0.00 | 0.00 | 0.00 | 0.00 | 0.00 | 0.00 |
| METRNL | 0.00 | 0.00 | 0.00 | 0.00 | 0.00 | 0.00 | 0.00 | 0.00 | 0.00 | 0.00 |
| MIF | 0.00 | 0.00 | 0.00 | 0.00 | 0.00 | 0.00 | 0.00 | 0.00 | 0.00 | 0.00 |
| MLANA | 3.79 | 0.00 | 0.00 | 0.00 | 0.00 | 0.00 | 0.00 | 0.00 | 0.00 | 0.00 |
| MLKL | 0.00 | 0.00 | 0.00 | 0.00 | 0.00 | 0.00 | 0.00 | 0.00 | 0.00 | 0.00 |
| MLLT3 | 0.00 | 0.00 | 0.00 | 0.00 | 0.00 | 0.00 | 0.00 | 0.00 | 0.00 | 0.00 |
| MMP12 | 0.00 | 0.00 | 0.00 | 0.00 | 0.00 | 0.00 | 0.00 | 0.00 | 0.00 | 0.00 |
| MMP8 | 0.00 | 0.00 | 0.00 | 0.00 | 0.00 | 0.00 | 0.00 | 0.00 | 0.00 | 0.00 |
| MMP9 | 0.00 | 0.00 | 0.00 | 0.00 | 0.00 | 0.00 | 0.00 | 0.00 | 0.00 | 0.00 |
| MNDA | 0.00 | 0.00 | 0.00 | 0.00 | 0.00 | 0.00 | 0.00 | 0.00 | 0.00 | 0.00 |
| MNDAL | 0.00 | 0.00 | 4.56 | 0.00 | 0.00 | 5.20 | 0.00 | 0.00 | 0.00 | 0.00 |
| MOB1A | 0.00 | 0.00 | 3.28 | 0.00 | 0.00 | 0.00 | 0.00 | 0.00 | 0.00 | 0.00 |
| MOB3C | 0.00 | 0.00 | 0.00 | 0.00 | 0.00 | 0.00 | 0.00 | 0.00 | 0.00 | 0.00 |
| MPEG1 | 3.96 | 0.00 | 0.00 | 0.00 | 0.00 | 0.00 | 0.00 | 0.00 | 0.00 | 0.00 |
| MPP1 | 0.00 | 0.00 | 0.00 | 0.00 | 0.00 | 0.00 | 0.00 | 0.00 | 0.00 | 0.00 |
| MRC1 | 0.00 | 0.00 | 0.00 | 0.00 | 0.00 | 0.00 | 0.00 | 0.00 | 0.00 | 0.00 |
| MRPL1 | 0.00 | 0.00 | 0.00 | 0.00 | 0.00 | 0.00 | 0.00 | 0.00 | 0.00 | 0.00 |
| MS4A1 | 0.00 | 0.00 | 3.25 | 0.00 | 0.00 | 0.00 | 0.00 | 0.00 | 0.00 | 0.00 |
| MS4A4B | 0.00 | 0.00 | 4.66 | 0.00 | 0.00 | 0.00 | 0.00 | 0.00 | 0.00 | 0.00 |
| MS4A4C | 0.00 | 0.00 | 0.00 | 0.00 | 0.00 | 0.00 | 0.00 | 0.00 | 0.00 | 0.00 |
| MS4A6B | 0.00 | 0.00 | 0.00 | 0.00 | 0.00 | 0.00 | 0.00 | 0.00 | 0.00 | 0.00 |
| MS4A6C | 0.00 | 0.00 | 0.00 | 0.00 | 0.00 | 0.00 | 0.00 | 0.00 | 0.00 | 0.00 |
| MS4A7 | 4.85 | 0.00 | 0.00 | 0.00 | 0.00 | 0.00 | 0.00 | 0.00 | 0.00 | 0.00 |
| MT1 | 0.00 | 0.00 | 0.00 | 0.00 | 0.00 | 5.94 | 0.00 | 0.00 | 0.00 | 0.00 |
| MT2 | 3.47 | 0.00 | 0.00 | 0.00 | 0.00 | 0.00 | 0.00 | 0.00 | 0.00 | 0.00 |
| MVP | 0.00 | 0.00 | 0.00 | 0.00 | 0.00 | 0.00 | 0.00 | 0.00 | 0.00 | 0.00 |
| MX1 | 0.00 | 0.00 | 0.00 | 0.00 | 0.00 | 0.00 | 0.00 | 0.00 | 0.00 | 0.00 |
| MXD1 | 0.00 | 0.00 | 9.10 | 0.00 | 0.00 | 0.00 | 0.00 | 0.00 | 0.00 | 0.00 |
| MYC | 0.00 | 0.00 | 0.00 | 0.00 | 0.00 | 0.00 | 0.00 | 0.00 | 0.00 | 0.00 |
| MYL4 | 0.00 | 0.00 | 0.00 | 0.00 | 0.00 | 0.00 | 0.00 | 0.00 | 0.00 | 0.00 |
| MYO5A | 0.00 | 0.00 | 0.00 | 0.00 | 0.00 | 0.00 | 0.00 | 0.00 | 0.00 | 0.00 |
| MZB1 | 0.00 | 0.00 | 3.54 | 0.00 | 0.00 | 0.00 | 0.00 | 0.00 | 0.00 | 8.85 |
| NAAA | 0.00 | 0.00 | 0.00 | 0.00 | 0.00 | 0.00 | 0.00 | 0.00 | 0.00 | 0.00 |
| NABP1 | 7.33 | 4.94 | 7.43 | 0.00 | 4.04 | 8.75 | 0.00 | 0.00 | 0.00 | 0.00 |
| NAPSA | 0.00 | 0.00 | 0.00 | 0.00 | 0.00 | 0.00 | 0.00 | 0.00 | 0.00 | 0.00 |
| NCEH1 | 0.00 | 0.00 | 0.00 | 0.00 | 0.00 | 0.00 | 0.00 | 0.00 | 0.00 | 0.00 |
| NCOA6 | 0.00 | 0.00 | 0.00 | 0.00 | 3.38 | 0.00 | 0.00 | 0.00 | 0.00 | 0.00 |
| NCOA7 | 0.00 | 0.00 | 0.00 | 0.00 | 0.00 | 0.00 | 0.00 | 0.00 | 0.00 | 0.00 |
| NCR1 | 0.00 | 0.00 | 3.82 | 0.00 | 0.00 | 0.00 | 0.00 | 0.00 | 0.00 | 0.00 |
| NEAT1 | 5.77 | 4.35 | 0.00 | 4.06 | 0.00 | 0.00 | 0.00 | 0.00 | 0.00 | 0.00 |
| NECAP1 | 0.00 | 0.00 | 0.00 | 0.00 | 0.00 | 0.00 | 0.00 | 0.00 | 0.00 | 0.00 |
| NEDD9 | 0.00 | 0.00 | 0.00 | 0.00 | 3.51 | 0.00 | 0.00 | 0.00 | 0.00 | 0.00 |
| NFKBIA | 0.00 | 0.00 | 0.00 | 5.09 | 0.00 | 0.00 | 0.00 | 0.00 | 0.00 | 0.00 |
| NFKBIE | 0.00 | 0.00 | 3.68 | 0.00 | 0.00 | 0.00 | 0.00 | 0.00 | 0.00 | 0.00 |
| NFKBIZ | 3.51 | 0.00 | 0.00 | 0.00 | 0.00 | 0.00 | 0.00 | 0.00 | 0.00 | 0.00 |
| NHSL2 | 0.00 | 0.00 | 0.00 | 0.00 | 0.00 | 0.00 | 0.00 | 0.00 | 0.00 | 0.00 |
| NINJ1 | 0.00 | 0.00 | 0.00 | 0.00 | 0.00 | 0.00 | 0.00 | 0.00 | 0.00 | 0.00 |
| NKG7 | 0.00 | 0.00 | 0.00 | 0.00 | 0.00 | 4.75 | 0.00 | 0.00 | 0.00 | 0.00 |
| NLRC5 | 0.00 | 0.00 | 0.00 | 0.00 | 0.00 | 0.00 | 0.00 | 0.00 | 0.00 | 0.00 |
| NMI | 0.00 | 0.00 | 0.00 | 0.00 | 0.00 | 0.00 | 0.00 | 0.00 | 0.00 | 0.00 |
| NOC4L | 0.00 | 0.00 | 0.00 | 0.00 | 0.00 | 0.00 | 0.00 | 0.00 | 0.00 | 0.00 |
| NPC2 | 0.00 | 0.00 | 0.00 | 0.00 | 0.00 | 0.00 | 0.00 | 0.00 | 0.00 | 0.00 |
| NR4A2 | 0.00 | 0.00 | 3.77 | 0.00 | 0.00 | 0.00 | 0.00 | 0.00 | 0.00 | 0.00 |
| NRBF2 | 0.00 | 0.00 | 0.00 | 0.00 | 0.00 | 0.00 | 0.00 | 0.00 | 0.00 | 0.00 |
| NRP1 | 0.00 | 0.00 | 0.00 | 0.00 | 3.30 | 0.00 | 0.00 | 0.00 | 0.00 | 0.00 |
| OAS1A | 0.00 | 0.00 | 0.00 | 0.00 | 0.00 | 0.00 | 0.00 | 0.00 | 0.00 | 0.00 |
| OAS1G | 0.00 | 0.00 | 0.00 | 0.00 | 0.00 | 0.00 | 0.00 | 0.00 | 0.00 | 0.00 |
| OAS2 | 0.00 | 0.00 | 0.00 | 0.00 | 0.00 | 0.00 | 0.00 | 0.00 | 0.00 | 0.00 |
| OASL1 | 0.00 | 0.00 | 0.00 | 0.00 | 0.00 | 0.00 | 0.00 | 0.00 | 0.00 | 0.00 |
| OASL2 | 0.00 | 0.00 | 0.00 | 0.00 | 0.00 | 0.00 | 0.00 | 0.00 | 0.00 | 0.00 |
| ODC1 | 0.00 | 0.00 | 0.00 | 0.00 | 0.00 | 3.69 | 0.00 | 0.00 | 0.00 | 5.04 |
| OSM | 0.00 | 0.00 | 0.00 | 0.00 | 0.00 | 0.00 | 0.00 | 0.00 | 0.00 | 0.00 |
| P2RY6 | 0.00 | 0.00 | 0.00 | 0.00 | 0.00 | 0.00 | 0.00 | 0.00 | 0.00 | 0.00 |
| PAK2 | 3.35 | 0.00 | 0.00 | 0.00 | 0.00 | 0.00 | 0.00 | 0.00 | 0.00 | 0.00 |
| PARP11 | 0.00 | 0.00 | 0.00 | 0.00 | 0.00 | 0.00 | 0.00 | 0.00 | 0.00 | 0.00 |
| PARP12 | 0.00 | 0.00 | 0.00 | 0.00 | 0.00 | 0.00 | 0.00 | 0.00 | 0.00 | 0.00 |
| PARP14 | 0.00 | 0.00 | 0.00 | 0.00 | 0.00 | 0.00 | 0.00 | 0.00 | 0.00 | 0.00 |
| PARP3 | 0.00 | 0.00 | 3.38 | 0.00 | 0.00 | 0.00 | 0.00 | 0.00 | 0.00 | 0.00 |
| PARP9 | 0.00 | 0.00 | 4.33 | 0.00 | 0.00 | 0.00 | 0.00 | 0.00 | 0.00 | 0.00 |
| PBK | 0.00 | 0.00 | 0.00 | 0.00 | 3.74 | 0.00 | 0.00 | 0.00 | 0.00 | 0.00 |
| PCNX | 0.00 | 0.00 | 0.00 | 0.00 | 0.00 | 0.00 | 0.00 | 0.00 | 0.00 | 0.00 |
| PDCD4 | 0.00 | 3.90 | 0.00 | 0.00 | 0.00 | 0.00 | 0.00 | 0.00 | 0.00 | 0.00 |
| PDE4B | 0.00 | 0.00 | 0.00 | 0.00 | 0.00 | 0.00 | 0.00 | 0.00 | 0.00 | 0.00 |
| PEX11G | 0.00 | 0.00 | 0.00 | 0.00 | 0.00 | 0.00 | 0.00 | 0.00 | 0.00 | 0.00 |
| PGLYRP1 | 0.00 | 0.00 | 0.00 | 0.00 | 0.00 | 0.00 | 0.00 | 0.00 | 0.00 | 0.00 |
| PGP | 0.00 | 0.00 | 0.00 | 0.00 | 0.00 | 0.00 | 0.00 | 0.00 | 0.00 | 0.00 |
| PGS1 | 0.00 | 0.00 | 0.00 | 0.00 | 0.00 | 0.00 | 0.00 | 0.00 | 0.00 | 0.00 |
| PHF11A | 0.00 | 0.00 | 0.00 | 0.00 | 3.71 | 0.00 | 0.00 | 0.00 | 0.00 | 0.00 |
| PHF11B | 0.00 | 0.00 | 5.31 | 0.00 | 0.00 | 0.00 | 0.00 | 0.00 | 0.00 | 0.00 |
| PHF11D | 0.00 | 0.00 | 0.00 | 0.00 | 0.00 | 0.00 | 0.00 | 0.00 | 0.00 | 0.00 |
| PHLDA1 | 0.00 | 0.00 | 0.00 | 4.41 | 0.00 | 0.00 | 0.00 | 0.00 | 0.00 | 0.00 |
| PIGM | 0.00 | 0.00 | 0.00 | 0.00 | 0.00 | 0.00 | 0.00 | 0.00 | 0.00 | 0.00 |
| PIK3AP1 | 0.00 | 0.00 | 0.00 | 0.00 | 0.00 | 0.00 | 0.00 | 0.00 | 0.00 | 0.00 |
| PIK3IP1 | 0.00 | 0.00 | 0.00 | 0.00 | 0.00 | 0.00 | 0.00 | 0.00 | 0.00 | 0.00 |
| PILRA | 0.00 | 0.00 | 0.00 | 0.00 | 0.00 | 0.00 | 0.00 | 0.00 | 0.00 | 0.00 |
| PIM1 | 0.00 | 0.00 | 0.00 | 0.00 | 0.00 | 0.00 | 0.00 | 0.00 | 0.00 | 0.00 |
| PIP4K2A | 0.00 | 0.00 | 0.00 | 0.00 | 0.00 | 0.00 | 0.00 | 0.00 | 0.00 | 0.00 |
| PIRB | 0.00 | 0.00 | 0.00 | 0.00 | 0.00 | 0.00 | 0.00 | 0.00 | 0.00 | 0.00 |
| PKIB | 0.00 | 0.00 | 0.00 | 0.00 | 0.00 | 0.00 | 0.00 | 0.00 | 4.32 | 0.00 |
| PKM | 0.00 | 0.00 | 0.00 | 0.00 | 0.00 | 6.20 | 0.00 | 0.00 | 0.00 | 0.00 |
| PLAC8 | 0.00 | 6.97 | 0.00 | 0.00 | 3.39 | 0.00 | 0.00 | 0.00 | 0.00 | 0.00 |
| PLAUR | 0.00 | 0.00 | 0.00 | 0.00 | 0.00 | 0.00 | 0.00 | 0.00 | 0.00 | 0.00 |
| PLBD2 | 0.00 | 3.85 | 0.00 | 0.00 | 0.00 | 0.00 | 0.00 | 0.00 | 0.00 | 0.00 |
| PLEK | 0.00 | 0.00 | 0.00 | 0.00 | 0.00 | 0.00 | 0.00 | 0.00 | 0.00 | 0.00 |
| PLEKHO2 | 0.00 | 0.00 | 0.00 | 0.00 | 0.00 | 0.00 | 0.00 | 0.00 | 0.00 | 0.00 |
| PLIN2 | 0.00 | 0.00 | 0.00 | 0.00 | 0.00 | 0.00 | 0.00 | 0.00 | 0.00 | 0.00 |
| PLK1 | 0.00 | 0.00 | 0.00 | 0.00 | 0.00 | 0.00 | 0.00 | 0.00 | 0.00 | 0.00 |
| PLK2 | 3.41 | 0.00 | 0.00 | 0.00 | 0.00 | 0.00 | 0.00 | 0.00 | 0.00 | 0.00 |
| PLK3 | 0.00 | 0.00 | 0.00 | 0.00 | 0.00 | 0.00 | 0.00 | 0.00 | 0.00 | 0.00 |
| PLP2 | 0.00 | 0.00 | 0.00 | 0.00 | 0.00 | 0.00 | 0.00 | 0.00 | 0.00 | 0.00 |
| PLSCR1 | 0.00 | 0.00 | 0.00 | 0.00 | 0.00 | 0.00 | 0.00 | 0.00 | 0.00 | 0.00 |
| PLTP | 0.00 | 0.00 | 0.00 | 0.00 | 0.00 | 0.00 | 0.00 | 0.00 | 0.00 | 0.00 |
| PMAIP1 | 0.00 | 0.00 | 0.00 | 0.00 | 0.00 | 0.00 | 0.00 | 0.00 | 0.00 | 0.00 |
| PML | 0.00 | 0.00 | 0.00 | 0.00 | 0.00 | 0.00 | 0.00 | 0.00 | 0.00 | 0.00 |
| PNP | 0.00 | 0.00 | 0.00 | 0.00 | 0.00 | 0.00 | 0.00 | 0.00 | 0.00 | 0.00 |
| PNRC1 | 0.00 | 0.00 | 0.00 | 0.00 | 0.00 | 0.00 | 0.00 | 0.00 | 0.00 | 0.00 |
| POU2F2 | 0.00 | 0.00 | 0.00 | 0.00 | 0.00 | 0.00 | 0.00 | 0.00 | 0.00 | 0.00 |
| PPP1R10 | 0.00 | 0.00 | 0.00 | 0.00 | 0.00 | 0.00 | 0.00 | 0.00 | 0.00 | 0.00 |
| PPP1R15A | 5.10 | 0.00 | 0.00 | 9.50 | 0.00 | 0.00 | 0.00 | 0.00 | 0.00 | 0.00 |
| PPP1R15B | 0.00 | 0.00 | 0.00 | 0.00 | 0.00 | 0.00 | 0.00 | 0.00 | 0.00 | 0.00 |
| PPP1R16B | 0.00 | 0.00 | 0.00 | 0.00 | 0.00 | 0.00 | 0.00 | 0.00 | 0.00 | 0.00 |
| PPP2R3C | 0.00 | 0.00 | 0.00 | 0.00 | 0.00 | 0.00 | 0.00 | 0.00 | 0.00 | 0.00 |
| PPP6R3 | 0.00 | 0.00 | 0.00 | 0.00 | 0.00 | 0.00 | 0.00 | 0.00 | 0.00 | 0.00 |
| PRDM2 | 0.00 | 0.00 | 0.00 | 0.00 | 0.00 | 0.00 | 0.00 | 0.00 | 0.00 | 0.00 |
| PRDX6 | 0.00 | 0.00 | 0.00 | 0.00 | 0.00 | 0.00 | 0.00 | 0.00 | 0.00 | 0.00 |
| PRELID2 | 0.00 | 0.00 | 3.89 | 0.00 | 0.00 | 0.00 | 0.00 | 0.00 | 0.00 | 0.00 |
| PRF1 | 0.00 | 0.00 | 4.06 | 0.00 | 0.00 | 0.00 | 0.00 | 0.00 | 0.00 | 0.00 |
| PRKX | 0.00 | 0.00 | 0.00 | 0.00 | 0.00 | 0.00 | 0.00 | 0.00 | 0.00 | 0.00 |
| PRR5 | 0.00 | 0.00 | 3.25 | 0.00 | 0.00 | 0.00 | 0.00 | 0.00 | 0.00 | 0.00 |
| PRR5L | 0.00 | 0.00 | 0.00 | 0.00 | 0.00 | 0.00 | 0.00 | 0.00 | 0.00 | 0.00 |
| PSAP | 0.00 | 0.00 | 0.00 | 0.00 | 0.00 | 0.00 | 0.00 | 0.00 | 0.00 | 0.00 |
| PSMB10 | 0.00 | 0.00 | 6.61 | 0.00 | 0.00 | 0.00 | 0.00 | 0.00 | 0.00 | 0.00 |
| PSMB8 | 0.00 | 0.00 | 0.00 | 0.00 | 0.00 | 0.00 | 0.00 | 0.00 | 0.00 | 0.00 |
| PSMB9 | 0.00 | 0.00 | 3.52 | 0.00 | 0.00 | 0.00 | 0.00 | 0.00 | 0.00 | 0.00 |
| PSME1 | 0.00 | 0.00 | 6.18 | 0.00 | 0.00 | 0.00 | 0.00 | 0.00 | 0.00 | 0.00 |
| PSME2 | 0.00 | 0.00 | 6.96 | 0.00 | 0.00 | 0.00 | 0.00 | 0.00 | 0.00 | 0.00 |
| PSME2B | 0.00 | 0.00 | 4.21 | 0.00 | 0.00 | 0.00 | 0.00 | 0.00 | 0.00 | 0.00 |
| PTAFR | 0.00 | 0.00 | 0.00 | 0.00 | 0.00 | 0.00 | 0.00 | 0.00 | 0.00 | 0.00 |
| PTGDS | 0.00 | 0.00 | 3.37 | 0.00 | 0.00 | 0.00 | 0.00 | 0.00 | 0.00 | 0.00 |
| PTGS2 | 0.00 | 0.00 | 0.00 | 0.00 | 0.00 | 0.00 | 0.00 | 0.00 | 0.00 | 0.00 |
| PTP4A1 | 0.00 | 0.00 | 0.00 | 0.00 | 0.00 | 0.00 | 0.00 | 0.00 | 0.00 | 0.00 |
| PTPN18 | 0.00 | 0.00 | 0.00 | 0.00 | 0.00 | 0.00 | 0.00 | 0.00 | 0.00 | 0.00 |
| PTPN22 | 0.00 | 0.00 | 0.00 | 0.00 | 0.00 | 0.00 | 0.00 | 0.00 | 0.00 | 0.00 |
| PTPRC | 0.00 | 0.00 | 0.00 | 0.00 | 0.00 | 0.00 | 0.00 | 0.00 | 0.00 | 0.00 |
| PTPRE | 0.00 | 0.00 | 0.00 | 0.00 | 0.00 | 0.00 | 0.00 | 0.00 | 0.00 | 0.00 |
| QTRTD1 | 0.00 | 0.00 | 0.00 | 0.00 | 0.00 | 0.00 | 0.00 | 0.00 | 0.00 | 0.00 |
| RAB7B | 0.00 | 0.00 | 0.00 | 0.00 | 0.00 | 0.00 | 0.00 | 0.00 | 0.00 | 0.00 |
| RACGAP1 | 0.00 | 0.00 | 0.00 | 0.00 | 0.00 | 0.00 | 0.00 | 0.00 | 0.00 | 0.00 |
| RAMP1 | 0.00 | 0.00 | 5.46 | 0.00 | 0.00 | 0.00 | 0.00 | 0.00 | 0.00 | 0.00 |
| RAP1B | 0.00 | 0.00 | 0.00 | 0.00 | 0.00 | 3.95 | 0.00 | 0.00 | 0.00 | 0.00 |
| RAPGEF2 | 0.00 | 0.00 | 0.00 | 0.00 | 0.00 | 0.00 | 0.00 | 0.00 | 0.00 | 0.00 |
| RAPGEF6 | 0.00 | 0.00 | 0.00 | 3.93 | 0.00 | 0.00 | 0.00 | 0.00 | 0.00 | 0.00 |
| RASA4 | 0.00 | 0.00 | 0.00 | 0.00 | 0.00 | 0.00 | 0.00 | 0.00 | 0.00 | 0.00 |
| RBM38 | 0.00 | 0.00 | 0.00 | 0.00 | 4.67 | 0.00 | 0.00 | 0.00 | 0.00 | 0.00 |
| RBM45 | 0.00 | 0.00 | 0.00 | 0.00 | 0.00 | 0.00 | 0.00 | 0.00 | 0.00 | 0.00 |
| RBPJ | 0.00 | 0.00 | 0.00 | 0.00 | 0.00 | 0.00 | 0.00 | 0.00 | 0.00 | 0.00 |
| RCHY1 | 3.65 | 0.00 | 0.00 | 0.00 | 0.00 | 0.00 | 0.00 | 0.00 | 0.00 | 0.00 |
| REL | 0.00 | 0.00 | 0.00 | 0.00 | 0.00 | 0.00 | 0.00 | 0.00 | 0.00 | 0.00 |
| RETNLG | 0.00 | 5.34 | 4.51 | 7.34 | 0.00 | 0.00 | 0.00 | 0.00 | 0.00 | 0.00 |
| RGCC | 0.00 | 0.00 | 0.00 | 0.00 | 0.00 | 0.00 | 0.00 | 0.00 | 0.00 | 0.00 |
| RGL1 | 0.00 | 0.00 | 0.00 | 0.00 | 0.00 | 0.00 | 0.00 | 0.00 | 0.00 | 0.00 |
| RGS1 | 0.00 | 0.00 | 0.00 | 5.88 | 0.00 | 0.00 | 0.00 | 0.00 | 0.00 | 0.00 |
| RHOV | 0.00 | 0.00 | 0.00 | 0.00 | 0.00 | 0.00 | 0.00 | 0.00 | 0.00 | 0.00 |
| RIN2 | 0.00 | 0.00 | 0.00 | 0.00 | 0.00 | 0.00 | 0.00 | 0.00 | 0.00 | 0.00 |
| RINL | 3.48 | 0.00 | 0.00 | 0.00 | 0.00 | 0.00 | 0.00 | 0.00 | 0.00 | 0.00 |
| RNASE6 | 0.00 | 0.00 | 0.00 | 0.00 | 0.00 | 0.00 | 0.00 | 0.00 | 6.16 | 0.00 |
| RNF138 | 6.14 | 0.00 | 0.00 | 0.00 | 0.00 | 0.00 | 0.00 | 0.00 | 0.00 | 0.00 |
| RNF213 | 0.00 | 0.00 | 0.00 | 0.00 | 0.00 | 0.00 | 0.00 | 0.00 | 0.00 | 0.00 |
| RNF219 | 0.00 | 0.00 | 0.00 | 0.00 | 3.42 | 0.00 | 0.00 | 0.00 | 0.00 | 0.00 |
| ROCK2 | 0.00 | 0.00 | 0.00 | 0.00 | 5.77 | 0.00 | 0.00 | 0.00 | 0.00 | 0.00 |
| RP23-6I17.1 | 0.00 | 0.00 | 0.00 | 0.00 | 0.00 | 0.00 | 0.00 | 0.00 | 0.00 | 0.00 |
| RPL13-PS3 | 0.00 | 0.00 | 0.00 | 0.00 | 0.00 | 0.00 | 0.00 | 0.00 | 0.00 | 0.00 |
| RPL22L1 | 0.00 | 0.00 | 0.00 | 0.00 | 0.00 | 0.00 | 0.00 | 0.00 | 0.00 | 0.00 |
| RPL35 | 0.00 | 0.00 | 0.00 | 0.00 | 0.00 | 0.00 | 0.00 | 0.00 | 0.00 | 0.00 |
| RPL36-PS3 | 0.00 | 0.00 | 0.00 | 0.00 | 0.00 | 0.00 | 0.00 | 0.00 | 0.00 | 0.00 |
| RPS10 | 0.00 | 0.00 | 0.00 | 0.00 | 0.00 | 0.00 | 0.00 | 0.00 | 0.00 | 0.00 |
| RPS18-PS3 | 0.00 | 0.00 | 0.00 | 0.00 | 0.00 | 0.00 | 0.00 | 0.00 | 0.00 | 0.00 |
| RRBP1 | 0.00 | 0.00 | 0.00 | 0.00 | 3.85 | 0.00 | 0.00 | 0.00 | 0.00 | 0.00 |
| RRM2 | 0.00 | 0.00 | 0.00 | 0.00 | 5.21 | 0.00 | 0.00 | 0.00 | 0.00 | 0.00 |
| RSAD2 | 0.00 | 4.58 | 0.00 | 0.00 | 0.00 | 0.00 | 0.00 | 0.00 | 0.00 | 0.00 |
| RSRP1 | 0.00 | 0.00 | 0.00 | 0.00 | 0.00 | 0.00 | 0.00 | 0.00 | 0.00 | 0.00 |
| RTN4IP1 | 0.00 | 0.00 | 0.00 | 0.00 | 0.00 | 0.00 | 0.00 | 0.00 | 0.00 | 0.00 |
| RTP4 | 6.61 | 6.52 | 12.08 | 3.73 | 0.00 | 4.17 | 0.00 | 0.00 | 0.00 | 0.00 |
| RUNX3 | 0.00 | 0.00 | 0.00 | 0.00 | 3.61 | 0.00 | 0.00 | 0.00 | 0.00 | 0.00 |
| S100A11 | 0.00 | 0.00 | 0.00 | 0.00 | 0.00 | 0.00 | 0.00 | 0.00 | 0.00 | 0.00 |
| S100A13 | 0.00 | 0.00 | 0.00 | 0.00 | 0.00 | 0.00 | 0.00 | 0.00 | 0.00 | 0.00 |
| S100A4 | 0.00 | 0.00 | 0.00 | 0.00 | 0.00 | 0.00 | 0.00 | 0.00 | 0.00 | 0.00 |
| S100A6 | 4.76 | 0.00 | 0.00 | 0.00 | 0.00 | 0.00 | 0.00 | 0.00 | 0.00 | 0.00 |
| S100A9 | 0.00 | 0.00 | 11.76 | 12.28 | 10.41 | 4.31 | 0.00 | 0.00 | 0.00 | 0.00 |
| S1PR1 | 0.00 | 0.00 | 0.00 | 0.00 | 0.00 | 0.00 | 5.14 | 0.00 | 0.00 | 0.00 |
| SAMHD1 | 0.00 | 0.00 | 9.09 | 0.00 | 0.00 | 3.97 | 0.00 | 0.00 | 0.00 | 0.00 |
| SAP30L | 0.00 | 0.00 | 0.00 | 0.00 | 3.28 | 0.00 | 0.00 | 0.00 | 0.00 | 0.00 |
| SAT1 | 0.00 | 0.00 | 0.00 | 0.00 | 0.00 | 0.00 | 0.00 | 0.00 | 0.00 | 0.00 |
| SDC3 | 0.00 | 0.00 | 0.00 | 0.00 | 0.00 | 0.00 | 0.00 | 0.00 | 0.00 | 0.00 |
| SDC4 | 0.00 | 0.00 | 9.13 | 0.00 | 0.00 | 7.01 | 0.00 | 0.00 | 4.72 | 0.00 |
| SDCBP | 0.00 | 0.00 | 0.00 | 0.00 | 0.00 | 0.00 | 0.00 | 0.00 | 0.00 | 0.00 |
| SDF4 | 0.00 | 0.00 | 0.00 | 0.00 | 0.00 | 0.00 | 0.00 | 0.00 | 0.00 | 4.16 |
| SDHAF1 | 0.00 | 0.00 | 0.00 | 0.00 | 0.00 | 0.00 | 0.00 | 0.00 | 0.00 | 0.00 |
| SELT | 0.00 | 0.00 | 3.75 | 0.00 | 0.00 | 0.00 | 0.00 | 0.00 | 0.00 | 0.00 |
| SENP6 | 0.00 | 0.00 | 0.00 | 0.00 | 4.04 | 0.00 | 0.00 | 0.00 | 0.00 | 0.00 |
| SEPN1 | 0.00 | 0.00 | 0.00 | 3.81 | 0.00 | 0.00 | 0.00 | 0.00 | 0.00 | 0.00 |
| SEPP1 | 0.00 | 0.00 | 0.00 | 0.00 | 0.00 | 0.00 | 0.00 | 0.00 | 0.00 | 0.00 |
| SEPT11 | 0.00 | 0.00 | 3.91 | 0.00 | 3.35 | 0.00 | 0.00 | 0.00 | 0.00 | 0.00 |
| SEPW1 | 0.00 | 0.00 | 0.00 | 0.00 | 0.00 | 0.00 | 0.00 | 0.00 | 0.00 | 0.00 |
| SERPINA3F | 0.00 | 0.00 | 4.18 | 0.00 | 0.00 | 0.00 | 0.00 | 0.00 | 0.00 | 0.00 |
| SERPINA3G | 3.77 | 0.00 | 3.67 | 0.00 | 0.00 | 0.00 | 0.00 | 0.00 | 0.00 | 0.00 |
| SERPINB1A | 0.00 | 0.00 | 0.00 | 0.00 | 0.00 | 0.00 | 0.00 | 0.00 | 0.00 | 0.00 |
| SERPINH1 | 0.00 | 0.00 | 0.00 | 0.00 | 0.00 | 0.00 | 0.00 | 0.00 | 0.00 | 0.00 |
| SERTAD1 | 3.77 | 0.00 | 0.00 | 0.00 | 0.00 | 0.00 | 0.00 | 0.00 | 0.00 | 0.00 |
| SETX | 3.74 | 0.00 | 4.00 | 0.00 | 0.00 | 0.00 | 0.00 | 0.00 | 0.00 | 0.00 |
| SFT2D1 | 0.00 | 0.00 | 3.59 | 0.00 | 0.00 | 0.00 | 0.00 | 0.00 | 0.00 | 0.00 |
| SGK3 | 0.00 | 0.00 | 0.00 | 0.00 | 5.05 | 0.00 | 0.00 | 0.00 | 0.00 | 0.00 |
| SGMS2 | 0.00 | 0.00 | 0.00 | 0.00 | 0.00 | 0.00 | 0.00 | 0.00 | 0.00 | 0.00 |
| SH2B2 | 3.57 | 0.00 | 0.00 | 0.00 | 0.00 | 0.00 | 0.00 | 0.00 | 0.00 | 0.00 |
| SHISA5 | 3.68 | 0.00 | 4.33 | 0.00 | 0.00 | 0.00 | 0.00 | 0.00 | 0.00 | 0.00 |
| SIPA1L1 | 0.00 | 0.00 | 0.00 | 0.00 | 3.34 | 0.00 | 0.00 | 0.00 | 0.00 | 0.00 |
| SLAMF7 | 3.36 | 0.00 | 0.00 | 0.00 | 0.00 | 0.00 | 0.00 | 0.00 | 0.00 | 0.00 |
| SLAMF8 | 0.00 | 0.00 | 0.00 | 0.00 | 0.00 | 0.00 | 0.00 | 0.00 | 0.00 | 0.00 |
| SLAMF9 | 0.00 | 0.00 | 0.00 | 0.00 | 0.00 | 0.00 | 0.00 | 0.00 | 0.00 | 0.00 |
| SLC12A9 | 0.00 | 0.00 | 0.00 | 0.00 | 0.00 | 0.00 | 0.00 | 0.00 | 0.00 | 0.00 |
| SLC17A5 | 0.00 | 0.00 | 0.00 | 0.00 | 0.00 | 0.00 | 4.64 | 0.00 | 0.00 | 0.00 |
| SLC26A2 | 0.00 | 0.00 | 0.00 | 0.00 | 0.00 | 0.00 | 0.00 | 0.00 | 0.00 | 0.00 |
| SLC28A2 | 0.00 | 0.00 | 0.00 | 0.00 | 0.00 | 0.00 | 0.00 | 0.00 | 0.00 | 0.00 |
| SLC2A6 | 0.00 | 0.00 | 0.00 | 0.00 | 0.00 | 0.00 | 0.00 | 0.00 | 0.00 | 0.00 |
| SLC30A1 | 0.00 | 0.00 | 0.00 | 0.00 | 0.00 | 0.00 | 0.00 | 0.00 | 0.00 | 0.00 |
| SLC35D1 | 0.00 | 0.00 | 0.00 | 0.00 | 3.90 | 0.00 | 0.00 | 0.00 | 0.00 | 0.00 |
| SLC36A3OS | 0.00 | 0.00 | 0.00 | 0.00 | 3.71 | 0.00 | 0.00 | 0.00 | 0.00 | 0.00 |
| SLC38A6 | 3.72 | 0.00 | 0.00 | 0.00 | 0.00 | 0.00 | 0.00 | 0.00 | 0.00 | 0.00 |
| SLC45A2 | 0.00 | 0.00 | 0.00 | 0.00 | 0.00 | 0.00 | 0.00 | 0.00 | 0.00 | 0.00 |
| SLC7A11 | 0.00 | 0.00 | 0.00 | 0.00 | 0.00 | 0.00 | 0.00 | 0.00 | 0.00 | 0.00 |
| SLFN1 | 0.00 | 0.00 | 7.66 | 0.00 | 0.00 | 0.00 | 0.00 | 0.00 | 0.00 | 0.00 |
| SLFN2 | 0.00 | 0.00 | 3.68 | 0.00 | 0.00 | 3.59 | 0.00 | 0.00 | 0.00 | 0.00 |
| SLFN5 | 4.65 | 0.00 | 0.00 | 0.00 | 0.00 | 0.00 | 0.00 | 0.00 | 0.00 | 0.00 |
| SLFN8 | 0.00 | 0.00 | 3.86 | 0.00 | 0.00 | 0.00 | 0.00 | 0.00 | 0.00 | 0.00 |
| SLFN9 | 0.00 | 0.00 | 0.00 | 0.00 | 0.00 | 0.00 | 0.00 | 0.00 | 0.00 | 0.00 |
| SLK | 0.00 | 0.00 | 0.00 | 0.00 | 0.00 | 0.00 | 0.00 | 0.00 | 0.00 | 0.00 |
| SLPI | 0.00 | 0.00 | 0.00 | 0.00 | 0.00 | 0.00 | 0.00 | 0.00 | 0.00 | 0.00 |
| SMIM14 | 0.00 | 0.00 | 0.00 | 0.00 | 0.00 | 0.00 | 0.00 | 0.00 | 4.30 | 0.00 |
| SMOX | 0.00 | 0.00 | 0.00 | 0.00 | 0.00 | 0.00 | 0.00 | 0.00 | 0.00 | 0.00 |
| SNN | 0.00 | 0.00 | 0.00 | 0.00 | 0.00 | 0.00 | 0.00 | 0.00 | 5.60 | 0.00 |
| SOCS1 | 0.00 | 0.00 | 5.71 | 0.00 | 0.00 | 0.00 | 0.00 | 0.00 | 0.00 | 0.00 |
| SOCS3 | 3.86 | 0.00 | 0.00 | 7.36 | 0.00 | 0.00 | 0.00 | 0.00 | 0.00 | 0.00 |
| SON | 0.00 | 0.00 | 0.00 | 0.00 | 0.00 | 0.00 | 0.00 | 0.00 | 0.00 | 0.00 |
| SOX4 | 0.00 | 0.00 | 0.00 | 0.00 | 0.00 | 0.00 | 0.00 | 0.00 | 0.00 | 0.00 |
| SP100 | 0.00 | 0.00 | 0.00 | 0.00 | 0.00 | 0.00 | 0.00 | 0.00 | 0.00 | 0.00 |
| SP110 | 0.00 | 0.00 | 0.00 | 0.00 | 0.00 | 0.00 | 0.00 | 0.00 | 0.00 | 0.00 |
| SP140 | 0.00 | 0.00 | 0.00 | 0.00 | 0.00 | 0.00 | 0.00 | 0.00 | 0.00 | 0.00 |
| SPI1 | 0.00 | 0.00 | 0.00 | 0.00 | 0.00 | 0.00 | 0.00 | 0.00 | 0.00 | 0.00 |
| SPIN2C | 0.00 | 0.00 | 4.33 | 0.00 | 0.00 | 0.00 | 0.00 | 0.00 | 0.00 | 0.00 |
| SPINT1 | 0.00 | 0.00 | 0.00 | 0.00 | 0.00 | 0.00 | 0.00 | 0.00 | 0.00 | 0.00 |
| SPP1 | 0.00 | 0.00 | 0.00 | 0.00 | 0.00 | 0.00 | 0.00 | 0.00 | 0.00 | 0.00 |
| SPRED1 | 0.00 | 0.00 | 0.00 | 0.00 | 0.00 | 0.00 | 0.00 | 0.00 | 0.00 | 0.00 |
| SPRY2 | 0.00 | 0.00 | 0.00 | 0.00 | 0.00 | 0.00 | 0.00 | 0.00 | 0.00 | 0.00 |
| SRGAP3 | 0.00 | 0.00 | 0.00 | 0.00 | 0.00 | 0.00 | 0.00 | 0.00 | 0.00 | 0.00 |
| SRGN | 0.00 | 0.00 | 0.00 | 0.00 | 0.00 | 0.00 | 0.00 | 0.00 | 0.00 | 0.00 |
| SRRM1 | 0.00 | 0.00 | 0.00 | 0.00 | 0.00 | 0.00 | 0.00 | 0.00 | 0.00 | 0.00 |
| SRSF5 | 0.00 | 0.00 | 0.00 | 0.00 | 0.00 | 0.00 | 0.00 | 0.00 | 0.00 | 0.00 |
| ST13 | 0.00 | 0.00 | 0.00 | 0.00 | 0.00 | 0.00 | 0.00 | 0.00 | 0.00 | 0.00 |
| ST3GAL6 | 0.00 | 0.00 | 0.00 | 0.00 | 0.00 | 0.00 | 0.00 | 0.00 | 0.00 | 0.00 |
| STAT2 | 0.00 | 0.00 | 0.00 | 0.00 | 0.00 | 0.00 | 0.00 | 0.00 | 0.00 | 0.00 |
| STX11 | 0.00 | 0.00 | 0.00 | 0.00 | 4.27 | 0.00 | 0.00 | 0.00 | 0.00 | 0.00 |
| SUB1 | 0.00 | 0.00 | 0.00 | 0.00 | 0.00 | 0.00 | 0.00 | 0.00 | 0.00 | 0.00 |
| SYNE1 | 0.00 | 0.00 | 0.00 | 0.00 | 0.00 | 0.00 | 0.00 | 0.00 | 0.00 | 0.00 |
| SYNJ1 | 0.00 | 0.00 | 0.00 | 0.00 | 0.00 | 0.00 | 0.00 | 0.00 | 0.00 | 0.00 |
| TAF15 | 0.00 | 0.00 | 0.00 | 0.00 | 4.13 | 0.00 | 0.00 | 0.00 | 0.00 | 0.00 |
| TALDO1 | 0.00 | 0.00 | 0.00 | 0.00 | 0.00 | 0.00 | 0.00 | 0.00 | 0.00 | 0.00 |
| TAPI | 0.00 | 0.00 | 7.56 | 0.00 | 0.00 | 0.00 | 0.00 | 0.00 | 0.00 | 0.00 |
| TAPBP | 0.00 | 0.00 | 4.49 | 0.00 | 0.00 | 0.00 | 0.00 | 0.00 | 0.00 | 0.00 |
| TAPBPL | 0.00 | 0.00 | 0.00 | 0.00 | 0.00 | 0.00 | 0.00 | 0.00 | 0.00 | 0.00 |
| TBX21 | 0.00 | 0.00 | 3.24 | 0.00 | 0.00 | 0.00 | 0.00 | 0.00 | 0.00 | 0.00 |
| TBXA2R | 0.00 | 0.00 | 0.00 | 0.00 | 0.00 | 0.00 | 0.00 | 0.00 | 0.00 | 0.00 |
| TCP11L2 | 0.00 | 0.00 | 0.00 | 0.00 | 0.00 | 0.00 | 0.00 | 0.00 | 4.35 | 0.00 |
| TDRD7 | 0.00 | 0.00 | 0.00 | 0.00 | 0.00 | 0.00 | 0.00 | 0.00 | 0.00 | 0.00 |
| TECPR1 | 0.00 | 0.00 | 0.00 | 0.00 | 0.00 | 0.00 | 0.00 | 0.00 | 0.00 | 0.00 |
| TESC | 0.00 | 0.00 | 0.00 | 0.00 | 0.00 | 0.00 | 0.00 | 0.00 | 0.00 | 0.00 |
| TEX30 | 0.00 | 0.00 | 3.45 | 0.00 | 0.00 | 0.00 | 0.00 | 0.00 | 0.00 | 0.00 |
| TGTP1 | 3.33 | 0.00 | 3.50 | 0.00 | 0.00 | 0.00 | 0.00 | 0.00 | 0.00 | 0.00 |
| TGTP2 | 4.76 | 0.00 | 9.21 | 0.00 | 0.00 | 4.26 | 0.00 | 0.00 | 0.00 | 0.00 |
| THBS1 | 0.00 | 0.00 | 0.00 | 0.00 | 0.00 | 0.00 | 0.00 | 0.00 | 0.00 | 0.00 |
| THEMIS2 | 4.56 | 0.00 | 0.00 | 0.00 | 0.00 | 0.00 | 0.00 | 0.00 | 0.00 | 0.00 |
| TIMP2 | 0.00 | 0.00 | 0.00 | 0.00 | 0.00 | 0.00 | 0.00 | 0.00 | 0.00 | 0.00 |
| TIPARP | 0.00 | 0.00 | 0.00 | 0.00 | 0.00 | 0.00 | 0.00 | 0.00 | 0.00 | 0.00 |
| TLE3 | 0.00 | 0.00 | 0.00 | 0.00 | 0.00 | 0.00 | 0.00 | 0.00 | 0.00 | 0.00 |
| TLK1 | 0.00 | 0.00 | 3.39 | 0.00 | 0.00 | 0.00 | 0.00 | 0.00 | 0.00 | 0.00 |
| TMEM173 | 0.00 | 0.00 | 3.27 | 0.00 | 0.00 | 0.00 | 0.00 | 0.00 | 0.00 | 0.00 |
| TMEM176A | 0.00 | 0.00 | 0.00 | 0.00 | 0.00 | 0.00 | 0.00 | 0.00 | 0.00 | 0.00 |
| TMEM176B | 0.00 | 0.00 | 0.00 | 0.00 | 0.00 | 0.00 | 0.00 | 0.00 | 0.00 | 0.00 |
| TMEM229B | 4.76 | 0.00 | 0.00 | 0.00 | 0.00 | 0.00 | 0.00 | 0.00 | 0.00 | 0.00 |
| TMSB10 | 0.00 | 0.00 | 0.00 | 0.00 | 0.00 | 0.00 | 0.00 | 0.00 | 0.00 | 0.00 |
| TMSB4X | 0.00 | 0.00 | 0.00 | 0.00 | 0.00 | 0.00 | 0.00 | 0.00 | 0.00 | 0.00 |
| TMX4 | 0.00 | 0.00 | 3.39 | 0.00 | 0.00 | 0.00 | 0.00 | 0.00 | 0.00 | 0.00 |
| TNFAIP3 | 3.34 | 0.00 | 0.00 | 0.00 | 0.00 | 0.00 | 0.00 | 0.00 | 0.00 | 0.00 |
| TNFRSF18 | 0.00 | 0.00 | 0.00 | 0.00 | 3.51 | 0.00 | 0.00 | 0.00 | 0.00 | 0.00 |
| TNFRSF4 | 0.00 | 0.00 | 0.00 | 0.00 | 0.00 | 0.00 | 0.00 | 0.00 | 0.00 | 9.81 |
| TNFRSF9 | 0.00 | 0.00 | 0.00 | 0.00 | 0.00 | 5.65 | 0.00 | 0.00 | 0.00 | 0.00 |
| TNFSF10 | 0.00 | 0.00 | 0.00 | 0.00 | 0.00 | 0.00 | 0.00 | 0.00 | 0.00 | 0.00 |
| TNIP3 | 0.00 | 0.00 | 0.00 | 0.00 | 0.00 | 0.00 | 0.00 | 0.00 | 0.00 | 0.00 |
| TNNI2 | 0.00 | 0.00 | 0.00 | 0.00 | 0.00 | 0.00 | 0.00 | 0.00 | 0.00 | 0.00 |
| TOB1 | 4.29 | 0.00 | 0.00 | 0.00 | 0.00 | 0.00 | 0.00 | 0.00 | 0.00 | 0.00 |
| TOMM34 | 0.00 | 0.00 | 0.00 | 0.00 | 0.00 | 0.00 | 0.00 | 0.00 | 0.00 | 0.00 |
| TOP2A | 0.00 | 0.00 | 0.00 | 0.00 | 0.00 | 0.00 | 0.00 | 0.00 | 5.30 | 0.00 |
| TOPBP1 | 0.00 | 0.00 | 0.00 | 0.00 | 4.76 | 0.00 | 0.00 | 0.00 | 0.00 | 0.00 |
| TOR1AIP1 | 0.00 | 0.00 | 3.17 | 0.00 | 3.93 | 0.00 | 0.00 | 0.00 | 0.00 | 0.00 |
| TOR3A | 0.00 | 0.00 | 0.00 | 0.00 | 0.00 | 0.00 | 0.00 | 0.00 | 0.00 | 0.00 |
| TPM3-RS7 | 0.00 | 0.00 | 0.00 | 0.00 | 0.00 | 0.00 | 0.00 | 0.00 | 0.00 | 0.00 |
| TPM4 | 0.00 | 0.00 | 5.28 | 0.00 | 0.00 | 0.00 | 0.00 | 0.00 | 0.00 | 0.00 |
| TPST1 | 0.00 | 0.00 | 0.00 | 0.00 | 0.00 | 0.00 | 0.00 | 0.00 | 0.00 | 0.00 |
| TRA2B | 6.01 | 0.00 | 3.26 | 8.24 | 0.00 | 0.00 | 0.00 | 0.00 | 0.00 | 0.00 |
| TRAF1 | 0.00 | 0.00 | 4.48 | 0.00 | 0.00 | 4.09 | 0.00 | 0.00 | 0.00 | 0.00 |
| TRAFD1 | 0.00 | 0.00 | 0.00 | 0.00 | 0.00 | 0.00 | 0.00 | 0.00 | 0.00 | 0.00 |
| TREM1 | 0.00 | 0.00 | 0.00 | 0.00 | 0.00 | 0.00 | 0.00 | 0.00 | 0.00 | 0.00 |
| TRIM12C | 0.00 | 0.00 | 3.18 | 0.00 | 0.00 | 0.00 | 0.00 | 0.00 | 0.00 | 0.00 |
| TRIM14 | 0.00 | 0.00 | 0.00 | 0.00 | 0.00 | 0.00 | 0.00 | 0.00 | 0.00 | 0.00 |
| TRIM30A | 0.00 | 0.00 | 3.33 | 0.00 | 0.00 | 0.00 | 0.00 | 0.00 | 0.00 | 0.00 |
| TRIM30B | 0.00 | 0.00 | 0.00 | 0.00 | 0.00 | 0.00 | 0.00 | 0.00 | 0.00 | 0.00 |
| TRIM30C | 0.00 | 0.00 | 0.00 | 0.00 | 0.00 | 0.00 | 0.00 | 0.00 | 0.00 | 0.00 |
| TRIM30D | 0.00 | 0.00 | 4.06 | 0.00 | 0.00 | 0.00 | 0.00 | 0.00 | 0.00 | 0.00 |
| TRIM37 | 0.00 | 0.00 | 0.00 | 0.00 | 3.91 | 0.00 | 0.00 | 0.00 | 0.00 | 0.00 |
| TRIM5 | 0.00 | 0.00 | 0.00 | 0.00 | 0.00 | 0.00 | 0.00 | 0.00 | 0.00 | 0.00 |
| TRIM56 | 6.38 | 0.00 | 0.00 | 0.00 | 0.00 | 0.00 | 0.00 | 0.00 | 0.00 | 0.00 |
| TRIP12 | 4.90 | 0.00 | 0.00 | 0.00 | 0.00 | 0.00 | 0.00 | 0.00 | 0.00 | 0.00 |
| TROVE2 | 0.00 | 0.00 | 0.00 | 0.00 | 0.00 | 0.00 | 0.00 | 0.00 | 0.00 | 0.00 |
| TSC22D3 | 5.05 | 0.00 | 0.00 | 0.00 | 4.89 | 0.00 | 0.00 | 0.00 | 4.97 | 0.00 |
| TSPAN13 | 0.00 | 0.00 | 3.73 | 0.00 | 0.00 | 0.00 | 0.00 | 0.00 | 0.00 | 0.00 |
| TSPO | 0.00 | 0.00 | 0.00 | 0.00 | 0.00 | 0.00 | 0.00 | 0.00 | 0.00 | 0.00 |
| TSTD3 | 0.00 | 0.00 | 0.00 | 0.00 | 0.00 | 0.00 | 0.00 | 0.00 | 0.00 | 0.00 |
| TTC37 | 0.00 | 0.00 | 0.00 | 0.00 | 5.52 | 0.00 | 0.00 | 0.00 | 0.00 | 0.00 |
| TUBA4A | 0.00 | 0.00 | 0.00 | 0.00 | 0.00 | 0.00 | 0.00 | 0.00 | 0.00 | 0.00 |
| TUBB5 | 0.00 | 0.00 | 0.00 | 0.00 | 0.00 | 0.00 | 0.00 | 0.00 | 0.00 | 0.00 |
| TXK | 0.00 | 0.00 | 0.00 | 0.00 | 0.00 | 0.00 | 0.00 | 0.00 | 0.00 | 0.00 |
| TXN1 | 0.00 | 0.00 | 0.00 | 0.00 | 0.00 | 0.00 | 0.00 | 0.00 | 0.00 | 0.00 |
| TXNIP | 0.00 | 0.00 | 0.00 | 0.00 | 0.00 | 0.00 | 0.00 | 0.00 | 0.00 | 0.00 |
| TYROBP | 4.76 | 0.00 | 0.00 | 0.00 | 0.00 | 0.00 | 0.00 | 0.00 | 0.00 | 0.00 |
| TYRP1 | 0.00 | 0.00 | 0.00 | 0.00 | 0.00 | 0.00 | 0.00 | 0.00 | 0.00 | 0.00 |
| UBB | 0.00 | 0.00 | 0.00 | 0.00 | 0.00 | 0.00 | 0.00 | 0.00 | 0.00 | 0.00 |
| UBC | 0.00 | 0.00 | 0.00 | 4.33 | 0.00 | 0.00 | 0.00 | 0.00 | 0.00 | 0.00 |
| UBE2C | 0.00 | 0.00 | 0.00 | 0.00 | 0.00 | 0.00 | 0.00 | 0.00 | 0.00 | 0.00 |
| UBE2G1 | 0.00 | 0.00 | 0.00 | 0.00 | 3.59 | 0.00 | 0.00 | 0.00 | 0.00 | 0.00 |
| UBE2L6 | 0.00 | 0.00 | 0.00 | 0.00 | 0.00 | 0.00 | 0.00 | 0.00 | 0.00 | 0.00 |
| UBP1 | 0.00 | 0.00 | 0.00 | 0.00 | 0.00 | 0.00 | 0.00 | 0.00 | 0.00 | 0.00 |
| UCK2 | 0.00 | 0.00 | 0.00 | 0.00 | 0.00 | 0.00 | 0.00 | 0.00 | 0.00 | 0.00 |
| UGCG | 0.00 | 0.00 | 0.00 | 0.00 | 4.94 | 0.00 | 0.00 | 0.00 | 0.00 | 0.00 |
| UNC93B1 | 0.00 | 0.00 | 0.00 | 0.00 | 0.00 | 0.00 | 0.00 | 0.00 | 0.00 | 0.00 |
| UPF3B | 0.00 | 0.00 | 0.00 | 0.00 | 0.00 | 0.00 | 0.00 | 0.00 | 0.00 | 0.00 |
| UPP1 | 0.00 | 0.00 | 0.00 | 0.00 | 0.00 | 0.00 | 0.00 | 0.00 | 0.00 | 0.00 |
| USP18 | 0.00 | 4.53 | 0.00 | 0.00 | 0.00 | 0.00 | 0.00 | 0.00 | 0.00 | 0.00 |
| USP25 | 0.00 | 0.00 | 0.00 | 0.00 | 0.00 | 0.00 | 0.00 | 0.00 | 0.00 | 0.00 |
| USP3 | 0.00 | 0.00 | 0.00 | 0.00 | 0.00 | 3.58 | 0.00 | 0.00 | 0.00 | 0.00 |
| USP9X | 0.00 | 0.00 | 0.00 | 0.00 | 3.64 | 0.00 | 0.00 | 0.00 | 0.00 | 0.00 |
| VAV3 | 3.37 | 0.00 | 0.00 | 0.00 | 0.00 | 0.00 | 0.00 | 0.00 | 0.00 | 0.00 |
| VCPIP1 | 0.00 | 0.00 | 3.66 | 0.00 | 0.00 | 0.00 | 0.00 | 0.00 | 0.00 | 0.00 |
| VEGFA | 0.00 | 0.00 | 0.00 | 0.00 | 0.00 | 0.00 | 0.00 | 0.00 | 0.00 | 0.00 |
| VPS37B | 0.00 | 0.00 | 0.00 | 0.00 | 0.00 | 0.00 | 0.00 | 0.00 | 0.00 | 0.00 |
| XDH | 0.00 | 0.00 | 0.00 | 0.00 | 0.00 | 0.00 | 0.00 | 0.00 | 0.00 | 0.00 |
| XPR1 | 0.00 | 0.00 | 0.00 | 0.00 | 0.00 | 0.00 | 0.00 | 0.00 | 0.00 | 0.00 |
| YAF2 | 0.00 | 0.00 | 0.00 | 0.00 | 0.00 | 0.00 | 0.00 | 0.00 | 0.00 | 0.00 |
| YWHAB | 0.00 | 0.00 | 0.00 | 0.00 | 0.00 | 0.00 | 0.00 | 0.00 | 0.00 | 0.00 |
| ZC3H7A | 0.00 | 0.00 | 0.00 | 0.00 | 3.69 | 0.00 | 0.00 | 0.00 | 0.00 | 0.00 |
| ZDHHC17 | 0.00 | 0.00 | 0.00 | 0.00 | 3.37 | 0.00 | 0.00 | 0.00 | 0.00 | 0.00 |
| ZDHHC18 | 0.00 | 0.00 | 0.00 | 0.00 | 0.00 | 0.00 | 0.00 | 0.00 | 0.00 | 0.00 |
| ZFP106 | 0.00 | 0.00 | 0.00 | 0.00 | 0.00 | 0.00 | 0.00 | 0.00 | 0.00 | 0.00 |
| ZFP36L2 | 0.00 | 0.00 | 0.00 | 0.00 | 0.00 | 0.00 | 0.00 | 0.00 | 0.00 | 0.00 |
| ZFP646 | 0.00 | 0.00 | 0.00 | 0.00 | 6.85 | 0.00 | 0.00 | 0.00 | 0.00 | 0.00 |
| ZFP729A | 0.00 | 0.00 | 0.00 | 0.00 | 3.82 | 0.00 | 0.00 | 0.00 | 0.00 | 0.00 |
| ZFP954 | 0.00 | 0.00 | 0.00 | 0.00 | 4.10 | 0.00 | 0.00 | 0.00 | 0.00 | 0.00 |
| ZFX | 0.00 | 0.00 | 0.00 | 0.00 | 0.00 | 0.00 | 0.00 | 0.00 | 0.00 | 0.00 |
| ZFYVE16 | 0.00 | 0.00 | 0.00 | 0.00 | 0.00 | 0.00 | 0.00 | 0.00 | 0.00 | 0.00 |
| ZNFX1 | 0.00 | 0.00 | 0.00 | 0.00 | 0.00 | 0.00 | 0.00 | 0.00 | 0.00 | 0.00 |
| ZUFSP | 0.00 | 0.00 | 3.24 | 0.00 | 0.00 | 0.00 | 0.00 | 0.00 | 0.00 | 0.00 |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. An isolated immune cell genetically modified ex vivo to decrease or eliminate expression or activity of opioid growth factor receptor (OGFr); or to decrease or eliminate expression or activity of proenkephalin (PENK) or proteolytic derivatives thereof (MENK and LENK).

2. (canceled)

3. The isolated immune cell of claim 1, wherein the immune cell is a CD8 T cell, optionally, wherein the CD8 T cell expresses a chimeric antigen receptor (CAR) or T cell receptor (TCR) specific for a tumor antigen; and/or
wherein the immune cell is a tumor infiltrating lymphocyte (TIL).

4. (canceled)

5. (canceled)

6. A method of enhancing anti-tumor immunity in a subject in need thereof comprising administering to the subject one or more isolated immune cells according to claim 1.

7. A method of enhancing anti-tumor immunity in a subject in need thereof comprising administering one or more agents capable of inhibiting opioid growth factor receptor (OGFr) signaling; or administering one or more agents capable of altering expression or activity of one or more genes selected from Table 2 or 3.

8. The method of claim 7, wherein the one or more agents is a small molecule antagonist of OGFr, optionally,
wherein the small molecule is selected from the group consisting of naloxone, naltrexone, methylnaltrexone (MNTX), and derivatives thereof; or
wherein the small molecule is selective for OGFr and does not antagonize the mu (MOR), delta (DOR), kappa (KOR) or nociceptin (NOR) opioid receptor subtypes; or
wherein the one or more agents comprise a small molecule degrader.

9. (canceled)

10. (canceled)

11. (canceled)

12. The method of claim 8, further comprising administering an opioid or opioid signaling agonist in combination with the selective OGFr antagonist.

13. The method of claim 7, wherein the one or more agents is an antibody to OGFr; or
wherein the one or more agents is an antibody to PENK or PENK-derived polypeptide; or
wherein the one or more agents is an engineered competitive PENK peptide, optionally, wherein the engineered competitive PENK peptide is derived PENK or MENK; or
wherein the one or more agents is an inhibitor of PENK proteolytic cleavage, optionally, wherein the inhibitor is a furin inhibitor; or wherein the inhibitor is an aminopeptidase inhibitor; or wherein the inhibitor is a cathepsin L (CTSL) inhibitor; or
wherein the one or more agent comprise a RNAi therapeutic that decreases the expression of PENK; or
wherein the one or more agents comprise a gene editing system that reduces PENK expression or function, optionally, wherein the gene editing system is a CRISPR-Cas system, a zinc finger nuclease, a TALEN, or a meganuclease.

14. (canceled)

15. (canceled)

16. (canceled)

17. (canceled)

18. (canceled)

19. The method of claim 13, wherein the furin inhibitor is selected from the group consisting of decanoyl-RVKR-chloromethylketone (CMK), hexa-D-arginine (D6R), and phenylacetyl-Arg-Val-Arg-4-amidinobenzylamide.

20. (canceled)

21. The method of claim 13, wherein the aminopeptidase inhibitor is bestatin.

22. (canceled)

23. The method of claim 13, wherein the cathepsin L (CTSL) inhibitor is selected from the group consisting of amantadine hydrochloride, teicoplanin, heparin, E64d, MDL28170, and KGP94.

24. (canceled)

25. (canceled)

26. (canceled)

27. The method of claim 13, wherein the CRISPR-Cas system is a base editing system or a prime editing system.

28. The method of claim 7, wherein the agent is administered directly to a tumor in the subject, or
wherein the agent is administered intravenously or intraperitoneally to the subject; or
wherein the subject is administered an additional immunotherapy.

29. (canceled)

30. (canceled)

31. The method of claim 28, wherein the immunotherapy comprises adoptive cell transfer, optionally, wherein adoptive cell transfer comprises the administration of T cells or natural killer cells that express a CAR (chimeric antigen receptor), T cells expressing a T cell receptor (TCR) specific for a tumor antigen, or tumor infiltrating lymphocytes (TILs).

32. (canceled)

33. The method of claim 28, wherein the immunotherapy comprises anti-PD-1, anti-CTLA4, anti-PD-L1, anti-TIM3, anti-TIGIT, anti-LAG3, or combinations thereof.

34. A method of monitoring tumor progression in a subject in need thereof comprising detecting expression of one or more genes selected from Table 2 or optionally, wherein the one or more genes is proenkephalin (PENK) or its proteolytic derivatives.

35. (canceled)

36. (canceled)

37. The method of claim 7, wherein the one or more genes are positively correlated with tumor size or time; or wherein the one or more genes are negatively correlated with tumor size or time.

38. The method of claim 37, wherein the one or more genes are upregulated in CD8+PD1+ TIM3+ T cells, optionally, wherein the one or more genes are upregulated in cluster T_4 or T_7.

39. (canceled)

40. The method of claim 37, wherein the one or more genes are upregulated in Tregs or cluster T_2.

41. (canceled)

42. The method of claim 6, wherein the cancer is selected from the group consisting of melanoma, renal cancer, glioma, thyroid cancer, lung cancer, liver cancer, pancreatic cancer, head and neck cancer, stomach cancer, colorectal cancer, urothelial cancer, prostate cancer, testicular cancer, breast cancer, cervical cancer, ovarian cancer and endometrial cancer.

43. The method of claim 7, wherein the cancer is selected from the group consisting of melanoma, renal cancer, glioma, thyroid cancer, lung cancer, liver cancer, pancreatic cancer, head and neck cancer, stomach cancer, colorectal cancer, urothelial cancer, prostate cancer, testicular cancer, breast cancer, cervical cancer, ovarian cancer and endometrial cancer.
